(19) United States
(12) Patent Application Publication
(10) Pub. No.: US 2006/0104989 A1
(43) Pub. Date: May 18, 2006

(54) Essential novel bacterial polypeptides

(75) Inventors: Aled Edwards, Toronto (CA); Akil Dharamsi, Richmond Hill (CA); Masoud Vedadi, Toronto (CA)

Correspondence Address: FOLEY HOAG, LLP;PATENT GROUP, WORLD TRADE CENTER WEST, 155 SEAPORT BLVD, BOSTON, MA, 02110, US

(73) Assignee: Affinium Pharmaceuticals, Inc., Toronto (CA)

(21) Appl. No.: 11/122,986

(22) Filed: May 5, 2005

Related U.S. Application Data
(63) Continuation of application No. PCT/CA03/01671, filed on Nov. 5, 2003.
(60) Provisional application No. 60/423,875, filed on Nov. 5, 2002.
(60) Provisional application No. 60/423,915, filed on Nov. 5, 2002.
(60) Provisional application No. 60/423,758, filed on Nov. 5, 2002.
(60) Provisional application No. 60/424,367, filed on Nov. 6, 2002.
(60) Provisional application No. 60/424,370, filed on Nov. 6, 2002.
(60) Provisional application No. 60/424,373, filed on Nov. 6, 2002.
(60) Provisional application No. 60/424,651, filed on Nov. 7, 2002.
(60) Provisional application No. 60/424,665, filed on Nov. 7, 2002.
(60) Provisional application No. 60/424,968, filed on Nov. 8, 2002.
(60) Provisional application No. 60/425,126, filed on Nov. 8, 2002.
(60) Provisional application No. 60/425,085, filed on Nov. 8, 2002.
(60) Provisional application No. 60/425,076, filed on Nov. 8, 2002.
(60) Provisional application No. 60/454,021, filed on Mar. 12, 2003.
(60) Provisional application No. 60/454,193, filed on Mar. 12, 2003.
(60) Provisional application No. 60/454,507, filed on Mar. 13, 2003.
(60) Provisional application No. 60/454,218, filed on Mar. 13, 2003.
(60) Provisional application No. 60/455,036, filed on Mar. 14, 2003.
(60) Provisional application No. 60/423,832, filed on Nov. 5, 2002.
(60) Provisional application No. 60/423,757, filed on Nov. 5, 2002.
(60) Provisional application No. 60/423,791, filed on Nov. 5, 2002.
(60) Provisional application No. 60/424,376, filed on Nov. 6, 2002.
(60) Provisional application No. 60/424,362, filed on Nov. 6, 2002.
(60) Provisional application No. 60/424,389, filed on Nov. 6, 2002.
(60) Provisional application No. 60/424,664, filed on Nov. 7, 2002.
(60) Provisional application No. 60/424,502, filed on Nov. 7, 2002.
(60) Provisional application No. 60/425,201, filed on Nov. 8, 2002.
(60) Provisional application No. 60/425,162, filed on Nov. 8, 2002.
(60) Provisional application No. 60/425,118, filed on Nov. 8, 2002.
(60) Provisional application No. 60/453,914, filed on Mar. 12, 2003.
(60) Provisional application No. 60/454,128, filed on Mar. 12, 2003.
(60) Provisional application No. 60/454,215, filed on Mar. 13, 2003.
(60) Provisional application No. 60/454,487, filed on Mar. 13, 2003.
(60) Provisional application No. 60/454,536, filed on Mar. 13, 2003.
(60) Provisional application No. 60/455,082, filed on Mar. 14, 2003.
(60) Provisional application No. 60/455,010, filed on Mar. 14, 2003.
(60) Provisional application No. 60/455,343, filed on Mar. 17, 2003.
(60) Provisional application No. 60/455,192, filed on Mar. 17, 2003.
(60) Provisional application No. 60/455,191, filed on Mar. 17, 2003.
(60) Provisional application No. 60/455,054, filed on Mar. 14, 2003.
(60) Provisional application No. 60/455,335, filed on Mar. 17, 2003.
(60) Provisional application No. 60/455,334, filed on Mar. 17, 2003.

(51) Int. Cl. A61K 39/02; C07K 14/21

(52) U.S. Cl. 424/190.1; 530/350

(57) ABSTRACT

The present invention relates to polypeptide targets for pathogenic bacteria. The invention also provides biochemical and biophysical characteristics of those polypeptides.

### RELATED APPLICATION INFORMATION

This application is a continuation of, and claims priority under 35 U.S.C. § 119 to, International Application No. PCT/CA03/01671, filed Nov. 5, 2003 which claims the benefit of priority to the following U.S. applications, as well as U.S. Ser. No. 60/455,191, filed Mar. 17, 2003, all of which applications are incorporated by reference in their entirety.

| Provisional Application | | |
|---|---|---|
| Number | Attorney Docket No. | Filing Date |
| 60/455,036 | IPT-433.60 | Mar. 14, 2003 |
| 60/423,875 | IPT-339.60 | Nov. 5, 2002 |
| 60/423,832 | IPT-345.60 | Nov. 5, 2002 |
| 60/423,915 | IPT-335.60 | Nov. 5, 2002 |
| 60/423,757 | IPT-331.60 | Nov. 5, 2002 |
| 60/423,758 | IPT-337.60 | Nov. 5, 2002 |
| 60/424,367 | IPT-357.60 | Nov. 6, 2002 |
| 60/424,376 | IPT-346.60 | Nov. 6, 2002 |
| 60/424,370 | IPT-347.60 | Nov. 6, 2002 |
| 60/424,362 | IPT-350.60 | Nov. 6, 2002 |
| 60/424,373 | IPT-356.60 | Nov. 6, 2002 |
| 60/424,389 | IPT-352.60 | Nov. 6, 2002 |
| 60/424,651 | IPT-343.60 | Nov. 7, 2002 |
| 60/424,664 | IPT-334.60 | Nov. 7, 2002 |
| 60/424,665 | IPT-332.60 | Nov. 7, 2002 |
| 60/424,502 | IPT-333.60 | Nov. 7, 2002 |
| 60/424,968 | IPT-340.60 | Nov. 8, 2002 |
| 60/425,201 | IPT-354.60 | Nov. 8, 2002 |
| 60/425,126 | IPT-358.60 | Nov. 8, 2002 |
| 60/425,162 | IPT-359.60 | Nov. 8, 2002 |
| 60/425,085 | IPT-336.60 | Nov. 8, 2002 |
| 60/425,118 | IPT-348.60 | Nov. 8, 2002 |
| 60/425,076 | IPT-355.60 | Nov. 8, 2002 |
| 60/454,215 | IPT-431.60 | Mar. 13, 2003 |
| 60/455,082 | IPT-442.60 | Mar. 14, 2003 |
| 60/455,334 | IPT-437.60 | Mar. 17, 2003 |
| 60/455,192 | IPT-455.60 | Mar. 17, 2003 |
| 60/423,791 | IPT-330.60 | Nov. 5, 2002 |
| 60/455,010 | IPT-448.60 | Mar. 14, 2003 |
| 60/454,507 | IPT-414.60 | Mar. 13, 2003 |
| 60/454,487 | IPT-428.60 | Mar. 13, 2003 |
| 60/453,914 | IPT-409.60 | Mar. 12, 2003 |
| 60/454,021 | IPT-410.60 | Mar. 12, 2003 |
| 60/454,128 | IPT-415.60 | Mar. 12, 2003 |
| 60/454,218 | IPT-429.60 | Mar. 13, 2003 |
| 60/454,536 | IPT-427.60 | Mar. 13, 2003 |
| 60/455,054 | IPT-439.60 | Mar. 14, 2003 |
| 60/455,335 | IPT-457.60 | Mar. 17, 2003 |
| 60/454,193 | IPT-411.60 | Mar. 12, 2003 |
| 60/455,343 | IPT-450.60 | Mar. 17, 2003 |

### INTRODUCTION

The discovery of novel antimicrobial agents that work by novel mechanisms is a problem researchers in all fields of drug development face today. The increasing prevalence of drug-resistant pathogens (bacteria, fungi, parasites, etc.) has led to significantly higher mortality rates from infectious diseases and currently presents a serious crisis worldwide. Despite the introduction of second and third generation antimicrobial drugs, certain pathogens have developed resistance to all currently available drugs.

One of the problems contributing to the development of multiple drug resistant pathogens is the limited number of protein targets for antimicrobial drugs. Many of the antibiotics currently in use are structurally related or act through common targets or pathways. Accordingly, adaptive mutation of a single gene may render a pathogenic species resistant to multiple classes of antimicrobial drugs. Therefore, the rapid discovery of drug targets is urgently needed in order to combat the constantly evolving threat by such infectious microorganisms.

Recent advances in bacterial and viral genomics research provides an opportunity for rapid progress in the identification of drug targets. The complete genomic sequences for a number of microorganisms are available. However, knowledge of the complete genomic sequence is only the first step in a long process toward discovery of a viable drug target. The genomic sequence must be annotated to identify open reading frames (ORFs), the essentiality of the protein encoded by the ORF must be determined and the mechanism of action of the gene product must be determined in order to develop a targeted approach to drug discovery.

There are a variety of computer programs available to annotate genomic sequences. Genome annotation involves both identification of genes as well assignment of function thereto based on sequence comparison to homologous proteins with known or predicted functions. However, genome annotation has turned out to be much more of an art than a science. Factors such as splice variants and sequencing errors coupled with the particular algorithms and databases used to annotate the genome can result in significantly different annotations for the same genome. For example, upon reanalysis of the genome of Mycoplasma pneumoniae using more rigorous sequence comparisons coupled with molecular biological techniques, such as gel electrophoresis and mass spectrometry, researchers were able to identify several previously unidentified coding sequences, to dismiss a previous identified coding sequence as a likely pseudogene, and to adjust the length of several previously defined ORFs (Dandkar et al. (2000) Nucl. Acids Res. 28(17): 3278-3288). Furthermore, while overall conservation between amino acid sequences generally indicates a conservation of structure and function, specific changes at key residues can lead to significant variation in the biochemical and biophysical properties of a protein. In a comparison of three different functional annotations of the Mycoplasma genitalium genome, it was discovered that some genes were assigned three different functions and it was estimated that the overall error rate in the annotations was at least 8% (Brenner (1999) Trends Genet 15(4): 132-3). Accordingly, molecular biological techniques are required to ensure proper genome annotation and identify valid drug targets.

However, confirmation of genome annotation using molecular biological techniques is not an easy proposition due to the unpredictability in expression and purification of polypeptide sequences. Further, in order to carry out structural studies to validate proteins as potential drug targets, it is generally necessary to modify the native proteins in order to facilitate these analyses, e.g., by labeling the protein (e.g., with a heavy atom, isotopic label, polypeptide tag, etc.) or by creating fragments of the polypeptide corresponding to functional domains of a multi-domain protein. Moreover, it is well-known that even small changes in the amino acid sequence of a protein may lead to dramatic affects on protein solubility (Eberstadt et al. (1998) Nature 392: 941-945). Accordingly, genome-wide validation of protein targets will require considerable effort even in light of the sequence of the entire genome of an organism and/or purification conditions for homologs of a particular target.

We have developed reliable, high throughput methods to address some of the shortcomings identified above. In part, using these methods, we have now identified, expressed, and purified a number of antimicrobial targets from S. aureus, H. pylori, E. coli, S. pneumoniae, E. faecalis and P. aeruginosa. Various biophysical, bioinformatic and biochemical studies have been used to characterize the polypeptides of the invention.

TABLE OF CONTENTS

| RELATED APPLICATION INFORMATION | | 1 |
|---|---|---|
| INTRODUCTION | | 2 |
| TABLE OF CONTENTS | | 4 |
| SUMMARY OF THE INVENTION | | 5 |
| BRIEF DESCRIPTION OF THE FIGURES | | 10 |
| DETAILED DESCRIPTION OF THE INVENTION | | 38 |
| 1. | Definitions | 38 |
| 2. | Polypeptides of the Invention | 56 |
| 3. | Nucleic Acids of the Invention | 83 |
| 4. | Homology Searching of Nucleotide and Polypeptide Sequences | 92 |
| 5. | Analysis of Protein Properties | 93 |
| | (a) Analysis of Proteins by Mass Spectrometry | 93 |
| | (b) Analysis of Proteins by Nuclear Magnetic Resonance (NMR) | 95 |
| | (c) Analysis of Proteins by X-ray Crystallography | 102 |
| 6. | Interacting Proteins | 118 |
| 7. | Antibodies | 132 |
| 8. | Diagnostic Assays | 135 |
| 9. | Drug Discovery | 138 |
| | (a) Drug Design | 139 |
| | (b) In Vitro Assays | 148 |
| | (c) In Vivo Assays | 149 |
| 10. | Vaccines | 151 |
| 11. | Array Analysis | 153 |
| 12. | Pharmaceutical Compositions | 156 |
| 13. | Antimicrobial Agents | 157 |

| EXEMPLIFICATION | | 158 |
|---|---|---|
| | EXAMPLE 1 Isolation and Cloning of Nucleic Acid | 158 |
| | EXAMPLE 2 Test Protein Expression and Solubility | 162 |
| | EXAMPLE 3 Native Protein Expression | 163 |
| | EXAMPLE 4 Expression of Selmet Labeled Polypeptides | 164 |
| | EXAMPLE 5 Expression of¹⁵N Labeled Polypeptides | 166 |
| | EXAMPLE 6 Method One for Purifying Polypeptides of the Invention | 167 |
| | EXAMPLE 7 Method Two for Purifying Polypeptides of the Invention | 168 |
| | EXAMPLE 8 Method Three for Purifying Polypeptides of the Invention | 169 |
| | EXAMPLE 9 Mass Spectrometry Analysis via Fingerprint Mapping | 170 |
| | EXAMPLE 10 Mass Spectrometry Analysis via High Mass | 172 |
| | EXAMPLE 11 Method One for Isolating and Identifying Interacting Proteins | 173 |
| | EXAMPLE 12 Method Two for Isolating and Identifying Interacting Proteins | 178 |
| | EXAMPLE 13 Sample for Mass Spectrometry of Interacting Proteins | 179 |
| | EXAMPLE 14 Mass Spectrometric Analysis of Interacting Proteins | 180 |
| | EXAMPLE 15 NMR Analysis | 182 |
| | EXAMPLE 16 X-ray Crystallography | 182 |
| | EXAMPLE 17 Annotations | 187 |
| | EXAMPLE 18 Essential Gene Analysis | 188 |
| | EXAMPLE 19 PDB Analysis | 189 |
| | EXAMPLE 20 Virtual Genome Analysis | 189 |
| | EXAMPLE 21 Epitopic Regions | 190 |

| | |
|---|---|
| EQUIVALENTS | 190 |
| CLAIMS | 194 |

### SUMMARY OF THE INVENTION

As part of an effort at genome-wide structural and functional characterization of microbial targets, the present invention provides polypeptides from S. aureus, H. pylori, E. coli, S. pneumoniae, E. faecalis and P. aeruginosa. In various aspects, the invention provides the nucleic acid and amino acid sequences of polypeptides of the invention. The invention also provides purified, soluble forms of polypeptides of the invention suitable for structural and functional characterization using a variety of techniques, including, for example, affinity chromatography, mass spectrometry, NMR and x-ray crystallography. The invention further provides modified versions of the polypeptides of the invention to facilitate characterization, including polypeptides labeled with isotopic or heavy atoms and fusion proteins. One or more crystallized forms of the polypeptides of the invention may also be provided.

In general, polypeptides of the invention are expected to be involved in bacterial viability. Because of the critical role that polypeptides with such functionality play in the life cycle and viability of their pathogenic species of origin, the polypeptides of the invention are, among other things, valuable drug targets. The biological activities for certain of the polypeptides of the invention are indicated in the following table, as described in further detail below.

| | | | Gene |
|---|---|---|---|
| SEQ ID NOS | Bacterial Species | Protein Annotation | Designation |
| SEQ ID NO: 5 | P. aeruginosa | GTP-binding | era |
| SEQ ID NO: 7 | | protein Era | |
| SEQ ID NO: 14 | E. faecalis | short chain | scd |
| SEQ ID NO: 16 | | dehydrogenase | |
| | | family protein | |
| SEQ ID NO: 23 | E. faecalis | glucose-inhibited | gidB |
| SEQ ID NO: 25 | | division protein B | |
| SEQ ID NO: 32 | E. faecalis | N utilization | nusB |
| SEQ ID NO: 34 | | substance protein B | |
| SEQ ID NO: 41 | E. faecalis | translation | tufA |
| SEQ ID NO: 43 | | elongation factor | |
| | | Tu | |
| SEQ ID NO: 50 | E. faecalis | GTP-binding | yhbZ (obg) |
| SEQ ID NO: 52 | | protein | |
| SEQ ID NO: 59 | S. pneumoniae | shikimate 5- | aroE |
| SEQ ID NO: 61 | | dehydrogenase | |
| SEQ ID NO: 68 | P. aeruginosa | conserved | b1983 |
| SEQ ID NO: 70 | | hypothetical | |
| | | protein | |
| SEQ ID NO: 77 | P. aeruginosa | beta-ketoacyl-ACP | fabB |
| SEQ ID NO: 79 | | synthase I | |
| SEQ ID NO: 86 | P. aeruginosa | ribosome recycling | frr |
| SEQ ID NO: 88 | | factor | |
| SEQ ID NO: 95 | S. pneumoniae | N utilization | nusA |
| SEQ ID NO: 97 | | substance protein A | |
| SEQ ID NO: 104 | P. aeruginosa | GTP-binding | yhbZ (obg) |
| SEQ ID NO: 106 | | protein | |
| SEQ ID NO: 113 | E. faecalis | acyl carrier protein | acpP |
| SEQ ID NO: 115 | | | |
| SEQ ID NO: 122 | E. faecalis | primosomal | dnaC |
| SEQ ID NO: 124 | | protein DnaI | |
| SEQ ID NO: 131 | E. faecalis | mannitol-1- | mtlD |
| SEQ ID NO: 133 | | phosphate 5- | |
| | | dehydrogenase | |
| SEQ ID NO: 140 | E. faecalis | DNA polymerase I | polA |
| SEQ ID NO: 142 | | | |
| SEQ ID NO: 149 | E. faecalis | adenylosuccinate | purB |
| SEQ ID NO: 151 | | lyase | |
| SEQ ID NO: 158 | S. aureus | adenylosuccinate | purB |
| SEQ ID NO: 160 | | lyase | |
| SEQ ID NO: 167 | S. pneumoniae | asparaginyl-tRNA | asnS |
| SEQ ID NO: 169 | | synthetase | |
| SEQ ID NO: 176 | S. pneumoniae | dihydrofolate | dfrA |
| SEQ ID NO: 178 | | reductase | |
| SEQ ID NO: 185 | E. faecalis | dnaJ protein | dnaJ |
| SEQ ID NO: 187 | | | |
| SEQ ID NO: 194 | P. aeruginosa | beta- | fabA |
| SEQ ID NO: 196 | | hydroxydecanoyl- | |
| | | ACP dehydrase | |
| SEQ ID NO: 203 | S. aureus | mannitol-1- | mtlD |
| SEQ ID NO: 205 | | phosphate 5- | |
| | | dehydrogenase | |
| SEQ ID NO: 212 | P. aeruginosa | dihydrofolate | dfrA |
| SEQ ID NO: 214 | | reductase | |
| SEQ ID NO: 221 | S. aureus | dihydrofolate | dfrA |
| SEQ ID NO: 223 | | reductase | |
| SEQ ID NO: 230 | S. aureus | replicative DNA | dnaB |
| SEQ ID NO: 232 | | helicase | |
| SEQ ID NO: 239 | P. aeruginosa | replicative DNA | dnaB |
| SEQ ID NO: 241 | | helicase | |
| SEQ ID NO: 248 | E. faecalis | cysteinyl-tRNA | cysS |
| SEQ ID NO: 250 | | synthetase | |
| SEQ ID NO: 257 | E. faecalis | oxidoreductase | fabG |
| SEQ ID NO: 259 | | | |
| SEQ ID NO: 266 | E. faecalis | cell division | ftsA |
| SEQ ID NO: 268 | | protein FtsA | |
| SEQ ID NO: 275 | H. pylori | UDP-3-0-acyl N- | lpxC |
| SEQ ID NO: 277 | | acetylglucosamine | |
| | | deacetylase | |
| SEQ ID NO: 284 | E. coli | UDP-3-O-(3- | lpxD |
| SEQ ID NO: 286 | | hydroxymyristoyl)- | |
| | | glucosamine N- | |
| | | acyltransferase | |
| SEQ ID NO: 293 | E. coli | glutamate | murI |
| SEQ ID NO: 295 | | racemase | |
| SEQ ID NO: 302 | E. faecalis | N utilization | nusA |
| SEQ ID NO: 304 | | substance protein A | |
| SEQ ID NO: 311 | H. pylori | transcription | nusA |
| SEQ ID NO: 313 | | termination factor | |
| | | NusA | |
| SEQ ID NO: 320 | H. pylori | transcription | nusB |
| SEQ ID NO: 322 | | termination factor | |
| SEQ ID NO: 329 | S. aureus | transcription | nusB |
| SEQ ID NO: 331 | | termination factor | |
| SEQ ID NO: 338 | S. pneumoniae | ATP-dependent | pcrA |
| SEQ ID NO: 340 | | DNA helicase | |
| SEQ ID NO: 347 | E. faecalis | DNA-directed | rpoA |
| SEQ ID NO: 349 | | RNA polymerase, | |
| | | alpha subunit | |
| SEQ ID NO: 356 | E. faecalis | RNA polymerase | rpoD |
| SEQ ID NO: 358 | | sigma-70 factor | |
| | | family protein | |

The SEQ ID NOS identified in the table above refer to the amino acid sequences for the indicated polypeptides, and such sequences are presented in full in the appended Figures. Other biological activities of polypeptides of the invention are described herein, or will be reasonably apparent to those skilled in the art in light of the present disclosure.

All of the information learned and described herein about the polypeptides of the invention may be used to design modulators of one or more of their biological activities. In particular, information critical to the design of therapeutic and diagnostic molecules, including, for example, the protein domain, druggable regions, structural information, and the like for polypeptides of the invention is now available or attainable as a result of the ability to prepare, purify and characterize them, and domains, fragments, variants and derivatives thereof.

In other aspects of the invention, structural and functional information about the polypeptides of the invention has and will be obtained. Such information, for example, may be incorporated into databases containing information on the polypeptides of the invention, as well as other polypeptide targets from other microbial species. Such databases will provide investigators with a powerful tool to analyze the polypeptides of the invention and aid in the rapid discovery and design of therapeutic and diagnostic molecules.

In another aspect, modulators, inhibitors, agonists or antagonists against the polypeptides of the invention, biological complexes containing them, or orthologues thereto, may be used to treat any disease or other treatable condition of a patient (including humans and animals). In particular, diseases caused by the following pathogenic species may be treated by any of such molecules:

| Bacterial Species | Diseases or Condition |
|---|---|
| S. aureus | a furuncle, chronic furunculosis, impetigo, acute |
| | osteomyelitis, pneumonia, endocarditis, scalded skin |
| | syndrome, toxic shock syndrome, and food poisoning |
| H. pylori | gastritis, adenocarcinoma and peptic ulcer disease |
| E. coli | urinary tract infection (e.g., cystitis or pyelonephritis), |
| | colitis, hemorrhagic colitis, diarrhea, and meningitis |
| | (particularly neonatal meningitis) |
| S. pneumoniae | pneumonia, meningitis, sinusitis, otitis media, |
| | endocarditis, arthritis, and peritonitis |
| P. aeruginosa | osteomyelitis, otitis externa, conjunctivitis, keratitis, |
| | endophthalmitis, alveolar necrosis, vascular invasion, |
| | bacteremia, and burn infection |
| E. faecalis | urinary tract infection, surgical wound infection, |
| | bacteremia, intra abdominal infection, pelvic infection, |
| | central nervous system infection, osteomyelitis, |
| | pulmonary infection, and endocarditis |

The present invention further allows relationships between polypeptides from the same and multiple species to be compared by isolating and studying the various polypeptides of the invention and other proteins. By such comparison studies, which may be multi-variable analysis as appropriate, it is possible to identify drugs that will affect multiple species or drugs that will affect one or a few species. In such a manner, so-called “wide spectrum” and “narrow spectrum” anti-infectives may be identified. Alternatively, drugs that are selective for one or more bacterial or other non-mammalian species, and not for one or more mammalian species (especially human), may be identified (and vice-versa).

In other embodiments, the invention contemplates kits including the subject nucleic acids, polypeptides, crystallized polypeptides, antibodies, and other subject materials, and optionally instructions for their use. Uses for such kits include, for example, diagnostic and therapeutic applications.

The embodiments and practices of the present invention, other embodiments, and their features and characteristics, will be apparent from the description, figures and claims that follow, with all of the claims hereby being incorporated by this reference into this Summary.

### BRIEF DESCRIPTION OF THE FIGURES

FIG. 1 shows the nucleic acid coding sequence (SEQ ID NO: 4) for GTP-binding protein Era, with gene designation of era, as predicted from the genomic sequence of P. aeruginosa. This predicted nucleic acid coding sequence was cloned and sequenced to produce the polynucleotide sequence shown in FIG. 3.

FIG. 2 shows the amino acid sequence (SEQ ID NO: 5) for GTP-binding protein Era (era) from P. aeruginosa, as predicted from the nucleotide sequence SEQ ID NO: 4 shown in FIG. 1.

FIG. 3 shows the experimentally determined nucleic acid coding sequence (SEQ ID NO: 6) for GTP-binding protein Era (era) from P. aeruginosa, as described in EXAMPLE 1.

FIG. 4 shows the amino acid sequence (SEQ ID NO: 7) for GTP-binding protein Era (era) from P. aeruginosa, as predicted from the experimentally determined nucleotide sequence SEQ ID NO: 6 shown in FIG. 3.

FIG. 5 shows the primer sequences used to amplify the nucleic acid of SEQ ID NO: 6. The primers are SEQ ID NO: 8 and SEQ ID NO: 9.

FIG. 6 contains TABLE 1, which provides among other things a variety of data and other information on GTP-binding protein Era (era) from P. aeruginosa.

FIG. 7 contains TABLE 2, which provides the results of several bioinformatic analyses relating to GTP-binding protein Era (era) from P. aeruginosa.

FIG. 8 depicts the results of tryptic peptide mass spectrum peak searching for GTP-binding protein Era (era) from P. aeruginosa, as described in EXAMPLE 9.

FIG. 9 depicts a MALDI-TOF mass spectrum of GTP-binding protein Era (era) from P. aeruginosa, as described in EXAMPLE 10.

FIG. 10 shows the nucleic acid coding sequence (SEQ ID NO: 13) for short chain dehydrogenase family protein, with gene designation of scd, as predicted from the genomic sequence of E. faecalis. This predicted nucleic acid coding sequence was cloned and sequenced to produce the polynucleotide sequence shown in FIG. 12.

FIG. 11 shows the amino acid sequence (SEQ ID NO: 14) for short chain dehydrogenase family protein (scd) from E. faecalis, as predicted from the nucleotide sequence SEQ ID NO: 13 shown in FIG. 10.

FIG. 12 shows the experimentally determined nucleic acid coding sequence (SEQ ID NO: 15) for short chain dehydrogenase family protein (scd) from E. faecalis, as described in EXAMPLE 1.

FIG. 13 shows the amino acid sequence (SEQ ID NO: 16) for short chain dehydrogenase family protein (scd) from E. faecalis, as predicted from the experimentally determined nucleotide sequence SEQ ID NO: 15 shown in FIG. 12.

FIG. 14 shows the primer sequences used to amplify the nucleic acid of SEQ ID NO: 15. The primers are SEQ ID NO: 17 and SEQ ID NO: 18.

FIG. 15 contains TABLE 3, which provides among other things a variety of data and other information on short chain dehydrogenase family protein (scd) from E. faecalis.

FIG. 16 contains TABLE 4, which provides the results of several bioinformatic analyses relating to short chain dehydrogenase family protein (scd) from E. faecalis.

FIG. 17 shows the nucleic acid coding sequence (SEQ ID NO: 22) for glucose-inhibited division protein B, with gene designation of gidB, as predicted from the genomic sequence of E. faecalis. This predicted nucleic acid coding sequence was cloned and sequenced to produce the polynucleotide sequence shown in FIG. 19.

FIG. 18 shows the amino acid sequence (SEQ ID NO: 23) for glucose-inhibited division protein B (gidB) from E. faecalis, as predicted from the nucleotide sequence SEQ ID NO: 22 shown in FIG. 17.

FIG. 19 shows the experimentally determined nucleic acid coding sequence (SEQ ID NO: 24) for glucose-inhibited division protein B (gidB) from E. faecalis, as described in EXAMPLE 1.

FIG. 20 shows the amino acid sequence (SEQ ID NO: 25) for glucose-inhibited division protein B (gidB) from E. faecalis, as predicted from the experimentally determined nucleotide sequence SEQ ID NO: 24 shown in FIG. 19.

FIG. 21 shows the primer sequences used to amplify the nucleic acid of SEQ ID NO: 24. The primers are SEQ ID NO: 26 and SEQ ID NO: 27.

FIG. 22 contains TABLE 5, which provides among other things a variety of data and other information on glucose-inhibited division protein B (gidB) from E. faecalis.

FIG. 23 contains TABLE 6, which provides the results of several bioinformatic analyses relating to glucose-inhibited division protein B (gidB) from E. faecalis.

FIG. 24 depicts a MALDI-TOF mass spectrum of glucose-inhibited division protein B (gidB) from E. faecalis, as described in EXAMPLE 10.

FIG. 25 shows the nucleic acid coding sequence (SEQ ID NO: 31) for N utilization substance protein B, with gene designation of nusB, as predicted from the genomic sequence of E. faecalis. This predicted nucleic acid coding sequence was cloned and sequenced to produce the polynucleotide sequence shown in FIG. 27.

FIG. 26 shows the amino acid sequence (SEQ ID NO: 32) for N utilization substance protein B (nusB) from E. faecalis, as predicted from the nucleotide sequence SEQ ID NO: 31 shown in FIG. 25.

FIG. 27 shows the experimentally determined nucleic acid coding sequence (SEQ ID NO: 33) for N utilization substance protein B (nusB) from E. faecalis, as described in EXAMPLE 1.

FIG. 28 shows the amino acid sequence (SEQ ID NO: 34) for N utilization substance protein B (nusB) from E. faecalis, as predicted from the experimentally determined nucleotide sequence SEQ ID NO: 33 shown in FIG. 27.

FIG. 29 shows the primer sequences used to amplify the nucleic acid of SEQ ID NO: 33. The primers are SEQ ID NO: 35 and SEQ ID NO: 36.

FIG. 30 contains TABLE 7, which provides among other things a variety of data and other information on N utilization substance protein B (nusB) from E. faecalis.

FIG. 31 contains TABLE 8, which provides the results of several bioinformatic analyses relating to N utilization substance protein B (nusB) from E. faecalis.

FIG. 32 depicts the results of tryptic peptide mass spectrum peak searching for N utilization substance protein B (nusB) from E. faecalis, as described in EXAMPLE 9.

FIG. 33 shows the nucleic acid coding sequence (SEQ ID NO: 40) for translation elongation factor Tu, with gene designation of tufA, as predicted from the genomic sequence of E. faecalis. This predicted nucleic acid coding sequence was cloned and sequenced to produce the polynucleotide sequence shown in FIG. 35.

FIG. 34 shows the amino acid sequence (SEQ ID NO: 41) for translation elongation factor Tu (tufA) from E. faecalis, as predicted from the nucleotide sequence SEQ ID NO: 40 shown in FIG. 33.

FIG. 35 shows the experimentally determined nucleic acid coding sequence (SEQ ID NO: 42) for translation elongation factor Tu (tufA) from E. faecalis, as described in EXAMPLE 1.

FIG. 36 shows the amino acid sequence (SEQ ID NO: 43) for translation elongation factor Tu (tufA) from E. faecalis, as predicted from the experimentally determined nucleotide sequence SEQ ID NO: 42 shown in FIG. 35.

FIG. 37 shows the primer sequences used to amplify the nucleic acid of SEQ ID NO: 42. The primers are SEQ ID NO: 44 and SEQ ID NO: 45.

FIG. 38 contains TABLE 9, which provides among other things a variety of data and other information on translation elongation factor Tu (tufA) from E. faecalis.

FIG. 39 contains TABLE 10, which provides the results of several bioinformatic analyses relating to translation elongation factor Tu (tufA) from E. faecalis.

FIG. 40 depicts the results of tryptic peptide mass spectrum peak searching for translation elongation factor Tu (tufA) from E. faecalis, as described in EXAMPLE 9.

FIG. 41 shows the nucleic acid coding sequence (SEQ ID NO: 49) for GTP-binding protein, with gene designation of yhbZ (obg), as predicted from the genomic sequence of E. faecalis. This predicted nucleic acid coding sequence was cloned and sequenced to produce the polynucleotide sequence shown in FIG. 43.

FIG. 42 shows the amino acid sequence (SEQ ID NO: 50) for GTP-binding protein (yhbZ (obg)) from E. faecalis, as predicted from the nucleotide sequence SEQ ID NO: 49 shown in FIG. 41.

FIG. 43 shows the experimentally determined nucleic acid coding sequence (SEQ ID NO: 51) for GTP-binding protein (yhbZ (obg)) from E. faecalis, as described in EXAMPLE 1.

FIG. 44 shows the amino acid sequence (SEQ ID NO: 52) for GTP-binding protein (yhbZ (obg)) from E. faecalis, as predicted from the experimentally determined nucleotide sequence SEQ ID NO: 51 shown in FIG. 43.

FIG. 45 shows the primer sequences used to amplify the nucleic acid of SEQ ID NO: 51. The primers are SEQ ID NO: 53 and SEQ ID NO: 54.

FIG. 46 contains TABLE 11, which provides among other things a variety of data and other information on GTP-binding protein yhbZ (obg)) from E. faecalis.

FIG. 47 contains TABLE 12, which provides the results of several bioinformatic analyses relating to GTP-binding protein (yhbZ (obg)) from E. faecalis.

FIG. 48 depicts the results of tryptic peptide mass spectrum peak searching for GTP-binding protein (yhbZ (obg)) from E. faecalis, as described in EXAMPLE 9.

FIG. 49 depicts a MALDI-TOF mass spectrum of GTP-binding protein (yhbZ (obg)) from E. faecalis, as described in EXAMPLE 10.

FIG. 50 shows the nucleic acid coding sequence (SEQ ID NO: 58) for shikimate 5-dehydrogenase, with gene designation of aroE, as predicted from the genomic sequence of S. pneumoniae. This predicted nucleic acid coding sequence was cloned and sequenced to produce the polynucleotide sequence shown in FIG. 52.

FIG. 51 shows the amino acid sequence (SEQ ID NO: 59) for shikimate 5-dehydrogenase (aroE) from S. pneumoniae, as predicted from the nucleotide sequence SEQ ID NO: 58 shown in FIG. 50.

FIG. 52 shows the experimentally determined nucleic acid coding sequence (SEQ ID NO: 60) for shikimate 5-dehydrogenase (aroE) from S. pneumoniae, as described in EXAMPLE 1.

FIG. 53 shows the amino acid sequence (SEQ ID NO: 61) for shikimate 5-dehydrogenase (aroE) from S. pneumoniae, as predicted from the experimentally determined nucleotide sequence SEQ ID NO: 60 shown in FIG. 52.

FIG. 54 shows the primer sequences used to amplify the nucleic acid of SEQ ID NO: 60. The primers are SEQ ID NO: 62 and SEQ ID NO: 63.

FIG. 55 contains TABLE 13, which provides among other things a variety of data and other information on shikimate 5-dehydrogenase (aroE) from S. pneumoniae.

FIG. 56 contains TABLE 14, which provides the results of several bioinformatic analyses relating to shikimate 5-dehydrogenase (aroE) from S. pneumoniae.

FIG. 57 shows the nucleic acid coding sequence (SEQ ID NO: 67) for conserved hypothetical protein, with gene designation of b1983, as predicted from the genomic sequence of P. aeruginosa. This predicted nucleic acid coding sequence was cloned and sequenced to produce the polynucleotide sequence shown in FIG. 59.

FIG. 58 shows the amino acid sequence (SEQ ID NO: 68) for conserved hypothetical protein (b1983) from P. aeruginosa, as predicted from the nucleotide sequence SEQ ID NO: 67 shown in FIG. 57.

FIG. 59 shows the experimentally determined nucleic acid coding sequence (SEQ ID NO: 69) for conserved hypothetical protein (b1983) from P. aeruginosa, as described in EXAMPLE 1.

FIG. 60 shows the amino acid sequence (SEQ ID NO: 70) for conserved hypothetical protein (b1983) from P. aeruginosa, as predicted from the experimentally determined nucleotide sequence SEQ ID NO: 69 shown in FIG. 59.

FIG. 61 shows the primer sequences used to amplify the nucleic acid of SEQ ID NO: 69. The primers are SEQ ID NO: 71 and SEQ ID NO: 72.

FIG. 62 contains TABLE 15, which provides among other things a variety of data and other information on conserved hypothetical protein (b1983) from P. aeruginosa.

FIG. 63 contains TABLE 16, which provides the results of several bioinformatic analyses relating to conserved hypothetical protein (b1983) from P. aeruginosa.

FIG. 64 shows the nucleic acid coding sequence (SEQ ID NO: 76) for beta-ketoacyl-ACP synthase L with gene designation of fabB, as predicted from the genomic sequence of P. aeruginosa. This predicted nucleic acid coding sequence was cloned and sequenced to produce the polynucleotide sequence shown in FIG. 66.

FIG. 65 shows the amino acid sequence (SEQ ID NO: 77) for beta-ketoacyl-ACP synthase I (fabB) from P. aeruginosa, as predicted from the nucleotide sequence SEQ ID NO: 76 shown in FIG. 64.

FIG. 66 shows the experimentally determined nucleic acid coding sequence (SEQ ID NO: 78) for beta-ketoacyl-ACP synthase I (fabB) from P. aeruginosa, as described in EXAMPLE 1.

FIG. 67 shows the amino acid sequence (SEQ ID NO: 79) for beta-ketoacyl-ACP synthase I (fabB) from P. aeruginosa, as predicted from the experimentally determined nucleotide sequence SEQ ID NO: 78 shown in FIG. 66.

FIG. 68 shows the primer sequences used to amplify the nucleic acid of SEQ ID NO: 78. The primers are SEQ ID NO: 80 and SEQ ID NO: 81.

FIG. 69 contains TABLE 17, which provides among other things a variety of data and other information on beta-ketoacyl-ACP synthase I (fabB) from P. aeruginosa.

FIG. 70 contains TABLE 18, which provides the results of several bioinformatic analyses relating to beta-ketoacyl-ACP synthase I (fabB) from P. aeruginosa.

FIG. 71 depicts the results of tryptic peptide mass spectrum peak searching for beta-ketoacyl-ACP synthase I (fabB) from P. aeruginosa, as described in EXAMPLE 9.

FIG. 72 shows the nucleic acid coding sequence (SEQ ID NO: 85) for ribosome recycling factor, with gene designation of frr, as predicted from the genomic sequence of P. aeruginosa. This predicted nucleic acid coding sequence was cloned and sequenced to produce the polynucleotide sequence shown in FIG. 74.

FIG. 73 shows the amino acid sequence (SEQ ID NO: 86) for ribosome recycling factor (frr) from P. aeruginosa, as predicted from the nucleotide sequence SEQ ID NO: 85 shown in FIG. 72.

FIG. 74 shows the experimentally determined nucleic acid coding sequence (SEQ ID NO: 87) for ribosome recycling factor (frr) from P. aeruginosa, as described in EXAMPLE 1.

FIG. 75 shows the amino acid sequence (SEQ ID NO: 88) for ribosome recycling factor (frr) from P. aeruginosa, as predicted from the experimentally determined nucleotide sequence SEQ ID NO: 87 shown in FIG. 74.

FIG. 76 shows the primer sequences used to amplify the nucleic acid of SEQ ID NO: 87. The primers are SEQ ID NO: 89 and SEQ ID NO: 90.

FIG. 77 contains TABLE 19, which provides among other things a variety of data and other information on ribosome recycling factor (frr) from P. aeruginosa.

FIG. 78 contains TABLE 20, which provides the results of several bioinformatic analyses relating to ribosome recycling factor (frr) from P. aeruginosa.

FIG. 79 depicts a ¹H, ¹⁵N Heteronuclear Single Quantum Coherence (HSQC) spectrum of ribosome recycling factor (frr) from P. aeruginosa, as described in EXAMPLE 15 below. The X-axis shows a proton chemical shift, while the Y-axis shows the ¹⁵N chemical shift of the purified ¹⁵N labeled polypeptide.

FIG. 80 depicts the results of tryptic peptide mass spectrum peak searching for ribosome recycling factor (frr) from P. aeruginosa, as described in EXAMPLE 9.

FIG. 81 depicts a MALDI-TOF mass spectrum of ribosome recycling factor (frr) from P. aeruginosa, as described in EXAMPLE 10.

FIG. 82 shows the nucleic acid coding sequence (SEQ ID NO: 94) for N utilization substance protein A, with gene designation of nusA, as predicted from the genomic sequence of S. pneumoniae. This predicted nucleic acid coding sequence was cloned and sequenced to produce the polynucleotide sequence shown in FIG. 84.

FIG. 83 shows the amino acid sequence (SEQ ID NO: 95) for N utilization substance protein A (nusA) from S. pneumoniae, as predicted from the nucleotide sequence SEQ ID NO: 94 shown in FIG. 82.

FIG. 84 shows the experimentally determined nucleic acid coding sequence (SEQ ID NO: 96) for N utilization substance protein A (nusA) from S. pneumoniae, as described in EXAMPLE 1.

FIG. 85 shows the amino acid sequence (SEQ ID NO: 97) for N utilization substance protein A (nusA) from S. pneumoniae, as predicted from the experimentally determined nucleotide sequence SEQ ID NO: 96 shown in FIG. 84.

FIG. 86 shows the primer sequences used to amplify the nucleic acid of SEQ ID NO: 96. The primers are SEQ ID NO: 98 and SEQ ID NO: 99.

FIG. 87 contains TABLE 21, which provides among other things a variety of data and other information on N utilization substance protein A (nusA) from S. pneumoniae.

FIG. 88 contains TABLE 22, which provides the results of several bioinformatic analyses relating to N utilization substance protein A (nusA) from S. pneumoniae.

FIG. 89 shows the nucleic acid coding sequence (SEQ ID NO: 103) for GTP-binding protein, with gene designation of yhbZ (obg), as predicted from the genomic sequence of P. aeruginosa. This predicted nucleic acid coding sequence was cloned and sequenced to produce the polynucleotide sequence shown in FIG. 91.

FIG. 90 shows the amino acid sequence (SEQ ID NO: 104) for GTP-binding protein (yhbZ (obg)) from P. aeruginosa, as predicted from the nucleotide sequence SEQ ID NO: 103 shown in FIG. 89.

FIG. 91 shows the experimentally determined nucleic acid coding sequence (SEQ ID NO: 105) for GTP-binding protein (yhbZ (obg)) from P. aeruginosa, as described in EXAMPLE 1.

FIG. 92 shows the amino acid sequence (SEQ ID NO: 106) for GTP-binding protein (yhbZ (obg)) from P. aeruginosa, as predicted from the experimentally determined nucleotide sequence SEQ ID NO: 105 shown in FIG. 91.

FIG. 93 shows the primer sequences used to amplify the nucleic acid of SEQ ID NO: 105. The primers are SEQ ID NO: 107 and SEQ ID NO: 108.

FIG. 94 contains TABLE 23, which provides among other things a variety of data and other information on GTP-binding protein (yhbZ (obg)) from P. aeruginosa.

FIG. 95 contains TABLE 24, which provides the results of several bioinformatic analyses relating to GTP-binding protein (yhbZ (obg)) from P. aeruginosa.

FIG. 96 depicts the results of tryptic peptide mass spectrum peak searching for GTP-binding protein (yhbZ (obg)) from P. aeruginosa, as described in EXAMPLE 9.

FIG. 97 depicts a MALDI-TOF mass spectrum of GTP-binding protein (yhbZ (obg)) from P. aeruginosa, as described in EXAMPLE 10.

FIG. 98 shows the nucleic acid coding sequence (SEQ ID NO: 112) for acyl carrier protein, with gene designation of acpP, as predicted from the genomic sequence of E. faecalis. This predicted nucleic acid coding sequence was cloned and sequenced to produce the polynucleotide sequence shown in FIG. 100.

FIG. 99 shows the amino acid sequence (SEQ ID NO: 113) for acyl carrier protein (acpP) from E. faecalis, as predicted from the nucleotide sequence SEQ ID NO: 112 shown in FIG. 98.

FIG. 100 shows the experimentally determined nucleic acid coding sequence (SEQ ID NO: 114) for acyl carrier protein (acpP) from E. faecalis, as described in EXAMPLE 1.

FIG. 101 shows the amino acid sequence (SEQ ID NO: 115) for acyl carrier protein (acpP) from E. faecalis, as predicted from the experimentally determined nucleotide sequence SEQ ID NO: 114 shown in FIG. 100.

FIG. 102 shows the primer sequences used to amplify the nucleic acid of SEQ ID NO: 114. The primers are SEQ ID NO: 116 and SEQ ID NO: 117.

FIG. 103 contains TABLE 25, which provides among other things a variety of data and other information on acyl carrier protein (acpP) from E. faecalis.

FIG. 104 contains TABLE 26, which provides the results of several bioinformatic analyses relating to acyl carrier protein (acpP) from E. faecalis.

FIG. 105 depicts a ¹H, ¹⁵N Heteronuclear Single Quantum Coherence (HSQC) spectrum of acyl carrier protein (acpP) from E. faecalis, as described in EXAMPLE 15 below. The X-axis shows a proton chemical shift, while the Y-axis shows the ¹⁵N chemical shift of the purified ¹⁵N labeled polypeptide.

FIG. 106 depicts a MALDI-TOF mass spectrum of acyl carrier protein (acpP) from E. faecalis, as described in EXAMPLE 10.

FIG. 107 shows the nucleic acid coding sequence (SEQ ID NO: 121) for primosomal protein DnaI, with gene designation of dnaC, as predicted from the genomic sequence of E. faecalis. This predicted nucleic acid coding sequence was cloned and sequenced to produce the polynucleotide sequence shown in FIG. 109.

FIG. 108 shows the amino acid sequence (SEQ ID NO: 122) for primosomal protein DnaI (dnaC) from E. faecalis, as predicted from the nucleotide sequence SEQ ID NO: 121 shown in FIG. 107.

FIG. 109 shows the experimentally determined nucleic acid coding sequence (SEQ ID NO: 123) for primosomal protein DnaI (dnaC) from E. faecalis, as described in EXAMPLE 1.

FIG. 110 shows the amino acid sequence (SEQ ID NO: 124) for primosomal protein DnaI (dnaC) from E. faecalis, as predicted from the experimentally determined nucleotide sequence SEQ ID NO: 123 shown in FIG. 109.

FIG. 111 shows the primer sequences used to amplify the nucleic acid of SEQ ID NO: 123. The primers are SEQ ID NO: 125 and SEQ ID NO: 126.

FIG. 112 contains TABLE 27, which provides among other things a variety of data and other information on primosomal protein DnaI (dnaC) from E. faecalis.

FIG. 113 contains TABLE 28, which provides the results of several bioinformatic analyses relating to primosomal protein DnaI (dnaC) from E. faecalis.

FIG. 114 depicts the results of tryptic peptide mass spectrum peak searching for primosomal protein DnaI (dnaC) from E. faecalis, as described in EXAMPLE 9.

FIG. 115 depicts a MALDI-TOF mass spectrum of primosomal protein DnaI (dnaC) from E. faecalis, as described in EXAMPLE 10.

FIG. 116 shows the nucleic acid coding sequence (SEQ ID NO: 130) for mannitol-1-phosphate 5-dehydrogenase, with gene designation of mtlD, as predicted from the genomic sequence of E. faecalis. This predicted nucleic acid coding sequence was cloned and sequenced to produce the polynucleotide sequence shown in FIG. 118.

FIG. 117 shows the amino acid sequence (SEQ ID NO: 131) for mannitol-1-phosphate 5-dehydrogenase (mtlD) from E. faecalis, as predicted from the nucleotide sequence SEQ ID NO: 130 shown in FIG. 116.

FIG. 118 shows the experimentally determined nucleic acid coding sequence (SEQ ID NO: 132) for mannitol-1-phosphate 5-dehydrogenase (mtlD) from E. faecalis, as described in EXAMPLE 1.

FIG. 119 shows the amino acid sequence (SEQ ID NO: 133) for mannitol-1-phosphate 5-dehydrogenase (mtlD) from E. faecalis, as predicted from the experimentally determined nucleotide sequence SEQ ID NO: 132 shown in FIG. 118.

FIG. 120 shows the primer sequences used to amplify the nucleic acid of SEQ ID NO: 132. The primers are SEQ ID NO: 134 and SEQ ID NO: 135.

FIG. 121 contains TABLE 29, which provides among other things a variety of data and other information on mannitol-1-phosphate 5-dehydrogenase (mtlD) from E. faecalis.

FIG. 122 contains TABLE 30, which provides the results of several bioinformatic analyses relating to mannitol-1-phosphate 5-dehydrogenase (mtlD) from E. faecalis.

FIG. 123 depicts the results of tryptic peptide mass spectrum peak searching for mannitol-1-phosphate 5-dehydrogenase (mtlD) from E. faecalis, as described in EXAMPLE 9.

FIG. 124 depicts a MALDI-TOF mass spectrum of mannitol-1-phosphate 5-dehydrogenase (mtlD) from E. faecalis, as described in EXAMPLE 10.

FIG. 125 shows the nucleic acid coding sequence (SEQ ID NO: 139) for DNA polymerase I, with gene designation of polA, as predicted from the genomic sequence of E. faecalis. This predicted nucleic acid coding sequence was cloned and sequenced to produce the polynucleotide sequence shown in FIG. 127.

FIG. 126 shows the amino acid sequence (SEQ ID NO: 140) for DNA polymerase I (polA) from E. faecalis, as predicted from the nucleotide sequence SEQ ID NO: 139 shown in FIG. 125.

FIG. 127 shows the experimentally determined nucleic acid coding sequence (SEQ ID NO: 141) for DNA polymerase I (polA) from E. faecalis, as described in EXAMPLE 1.

FIG. 128 shows the amino acid sequence (SEQ ID NO: 142) for DNA polymerase I (polA) from E. faecalis, as predicted from the experimentally determined nucleotide sequence SEQ ID NO: 141 shown in FIG. 127.

FIG. 129 shows the primer sequences used to amplify the nucleic acid of SEQ ID NO: 141. The primers are SEQ ID NO: 143 and SEQ ID NO: 144.

FIG. 130 contains TABLE 31, which provides among other things a variety of data and other information on DNA polymerase I (polA) from E. faecalis.

FIG. 131 contains TABLE 32, which provides the results of several bioinformatic analyses relating to DNA polymerase I (polA) from E. faecalis.

FIG. 132 depicts the results of tryptic peptide mass spectrum peak searching for DNA polymerase I (polA) from E. faecalis, as described in EXAMPLE 9.

FIG. 133 depicts a MALDI-TOF mass spectrum of DNA polymerase I (polA) from E. faecalis, as described in EXAMPLE 10.

FIG. 134 shows the nucleic acid coding sequence (SEQ ID NO: 148) for adenylosuccinate lyase, with gene designation of purB, as predicted from the genomic sequence of E. faecalis. This predicted nucleic acid coding sequence was cloned and sequenced to produce the polynucleotide sequence shown in FIG. 136.

FIG. 135 shows the amino acid sequence (SEQ ID NO: 149) for adenylosuccinate lyase (purB) from E. faecalis, as predicted from the nucleotide sequence SEQ ID NO: 148 shown in FIG. 134.

FIG. 136 shows the experimentally determined nucleic acid coding sequence (SEQ ID NO: 150) for adenylosuccinate lyase (purB) from E. faecalis, as described in EXAMPLE 1.

FIG. 137 shows the amino acid sequence (SEQ ID NO: 151) for adenylosuccinate lyase (purB) from E. faecalis, as predicted from the experimentally determined nucleotide sequence SEQ ID NO: 150 shown in FIG. 136.

FIG. 138 shows the primer sequences used to amplify the nucleic acid of SEQ ID NO: 150. The primers are SEQ ID NO: 152 and SEQ ID NO: 153.

FIG. 139 contains TABLE 33, which provides among other things a variety of data and other information on adenylosuccinate lyase (purB) from E. faecalis.

FIG. 140 contains TABLE 34, which provides the results of several bioinformatic analyses relating to adenylosuccinate lyase (purB) from E. faecalis.

FIG. 141 depicts the results of tryptic peptide mass spectrum peak searching for adenylosuccinate lyase (purB) from E. faecalis, as described in EXAMPLE 9.

FIG. 142 depicts a MALDI-TOF mass spectrum of adenylosuccinate lyase (purB) from E. faecalis, as described in EXAMPLE 10.

FIG. 143 shows the nucleic acid coding sequence (SEQ ID NO: 157) for adenylosuccinate lyase, with gene designation of purB, as predicted from the genomic sequence of S. aureus. This predicted nucleic acid coding sequence was cloned and sequenced to produce the polynucleotide sequence shown in FIG. 145.

FIG. 144 shows the amino acid sequence (SEQ ID NO: 158) for adenylosuccinate lyase (purB) from S. aureus, as predicted from the nucleotide sequence SEQ ID NO: 157 shown in FIG. 143.

FIG. 145 shows the experimentally determined nucleic acid coding sequence (SEQ ID NO: 159) for adenylosuccinate lyase (purB) from S. aureus, as described in EXAMPLE 1.

FIG. 146 shows the amino acid sequence (SEQ ID NO: 160) for adenylosuccinate lyase (curb) from S. aureus, as predicted from the experimentally determined nucleotide sequence SEQ ID NO: 159 shown in FIG. 145.

FIG. 147 shows the primer sequences used to amplify the nucleic acid of SEQ ID NO: 159. The primers are SEQ ID NO: 161 and SEQ ID NO: 162.

FIG. 148 contains TABLE 35, which provides among other things a variety of data and other information on adenylosuccinate lyase (purB) from S. aureus.

FIG. 149 contains TABLE 36, which provides the results of several bioinformatic analyses relating to adenylosuccinate lyase (purB) from S. aureus.

FIG. 150 depicts the results of tryptic peptide mass spectrum peak searching for adenylosuccinate lyase (purB) from S. aureus, as described in EXAMPLE 9.

FIG. 151 shows the nucleic acid coding sequence (SEQ ID NO: 166) for asparaginyl-tRNA synthetase, with gene designation of asnS, as predicted from the genomic sequence of S. pneumoniae. This predicted nucleic acid coding sequence was cloned and sequenced to produce the polynucleotide sequence shown in FIG. 153.

FIG. 152 shows the amino acid sequence (SEQ ID NO: 167) for asparaginyl-tRNA synthetase (asnS) from S. pneumoniae, as predicted from the nucleotide sequence SEQ ID NO: 166 shown in FIG. 151.

FIG. 153 shows the experimentally determined nucleic acid coding sequence (SEQ ID NO: 168) for asparaginyl-tRNA synthetase (asnS) from S. pneumoniae, as described in EXAMPLE 1.

FIG. 154 shows the amino acid sequence (SEQ ID NO: 169) for asparaginyl-tRNA synthetase (asnS) from S. pneumoniae, as predicted from the experimentally determined nucleotide sequence SEQ ID NO: 168 shown in FIG. 153.

FIG. 155 shows the primer sequences used to amplify the nucleic acid of SEQ ID NO: 168. The primers are SEQ ID NO: 170 and SEQ ID NO: 171.

FIG. 156 contains TABLE 37, which provides among other things a variety of data and other information on asparaginyl-tRNA synthetase (asnS) from S. pneumoniae.

FIG. 157 contains TABLE 38, which provides the results of several bioinformatic analyses relating to asparaginyl-tRNA synthetase (asnS) from S. pneumoniae.

FIG. 158 depicts the results of tryptic peptide mass spectrum peak searching for asparaginyl-tRNA synthetase (asnS) from S. pneumoniae, as described in EXAMPLE 9.

FIG. 159 shows the nucleic acid coding sequence (SEQ ID NO: 175) for dihydrofolate reductase, with gene designation of dfrA, as predicted from the genomic sequence of S. pneumoniae. This predicted nucleic acid coding sequence was cloned and sequenced to produce the polynucleotide sequence shown in FIG. 161.

FIG. 160 shows the amino acid sequence (SEQ ID NO: 176) for dihydrofolate reductase (dfrA) from S. pneumoniae, as predicted from the nucleotide sequence SEQ ID NO: 175 shown in FIG. 159.

FIG. 161 shows the experimentally determined nucleic acid coding sequence (SEQ ID NO: 177) for dihydrofolate reductase (dfrA) from S. pneumoniae, as described in EXAMPLE 1.

FIG. 162 shows the amino acid sequence (SEQ ID NO: 178) for dihydrofolate reductase (dfrA) from S. pneumoniae, as predicted from the experimentally determined nucleotide, sequence SEQ ID NO: 177 shown in FIG. 161.

FIG. 163 shows the primer sequences used to amplify the nucleic acid of SEQ ID NO: 177. The primers are SEQ ID NO: 179 and SEQ ID NO: 180.

FIG. 164 contains TABLE 39, which provides among other things a variety of data and other information on dihydrofolate reductase (dfrA) from S. pneumoniae.

FIG. 165 contains TABLE 40, which provides the results of several bioinformatic analyses relating to dihydrofolate reductase (dfrA) from S. pneumoniae.

FIG. 166 depicts the results of tryptic peptide mass spectrum peak searching for dihydrofolate reductase (dfrA) from S. pneumoniae, as described in EXAMPLE 9.

FIG. 167 depicts a MALDI-TOF mass spectrum of dihydrofolate reductase (dfrA) from S. pneumoniae, as described in EXAMPLE 10.

FIG. 168 shows the nucleic acid coding sequence (SEQ ID NO: 184) for dnaJ protein, with gene designation of dnaJ, as predicted from the genomic sequence of E. faecalis. This predicted nucleic acid coding sequence was cloned and sequenced to produce the polynucleotide sequence shown in FIG. 170.

FIG. 169 shows the amino acid sequence (SEQ ID NO: 185) for dnaJ protein (dnaJ) from E. faecalis, as predicted from the nucleotide sequence SEQ ID NO: 184 shown in FIG. 168.

FIG. 170 shows the experimentally determined nucleic acid coding sequence (SEQ ID NO: 186) for dnaJ protein (dnaJ) from E. faecalis, as described in EXAMPLE 1.

FIG. 171 shows the amino acid sequence (SEQ ID NO: 187) for dnaJ protein (dnaJ) from E. faecalis, as predicted from the experimentally determined nucleotide sequence SEQ ID NO: 186 shown in FIG. 170.

FIG. 172 shows the primer sequences used to amplify the nucleic acid of SEQ ID NO: 186. The primers are SEQ ID NO: 188 and SEQ ID NO: 189.

FIG. 173 contains TABLE 41, which provides among other things a variety of data and other information on dnaJ protein (dnaJ) from E. faecalis.

FIG. 174 contains TABLE 42, which provides the results of several bioinformatic analyses relating to dnaJ protein (dnaJ) from E. faecalis.

FIG. 175 shows the nucleic acid coding sequence (SEQ ID NO: 193) for beta-hydroxydecanoyl-ACP dehydrase, with gene designation of fabA, as predicted from the genomic sequence of P. aeruginosa. This predicted nucleic acid coding sequence was cloned and sequenced to produce the polynucleotide sequence shown in FIG. 177.

FIG. 176 shows the amino acid sequence (SEQ ID NO: 194) for beta-hydroxydecanoyl-ACP dehydrase (fabA) from P. aeruginosa, as predicted from the nucleotide sequence SEQ ID NO: 193 shown in FIG. 175.

FIG. 177 shows the experimentally determined nucleic acid coding sequence (SEQ ID NO: 195) for beta-hydroxydecanoyl-ACP dehydrase (fabA) from P. aeruginosa, as described in EXAMPLE 1.

FIG. 178 shows the amino acid sequence (SEQ ID NO: 196) for beta-hydroxydecanoyl-ACP dehydrase (fabA) from P. aeruginosa, as predicted from the experimentally determined nucleotide sequence SEQ ID NO: 195 shown in FIG. 177.

FIG. 179 shows the primer sequences used to amplify the nucleic acid of SEQ ID NO: 195. The primers are SEQ ID NO: 197 and SEQ ID NO: 198.

FIG. 180 contains TABLE 43, which provides among other things a variety of data and other information on beta-hydroxydecanoyl-ACP dehydrase (fabA) from P. aeruginosa.

FIG. 181 contains TABLE 44, which provides the results of several bioinformatic analyses relating to beta-hydroxydecanoyl-ACP dehydrase (fabA) from P. aeruginosa.

FIG. 182 depicts a MALDI-TOF mass spectrum of beta-hydroxydecanoyl-ACP dehydrase (fabA) from P. aeruginosa, as described in EXAMPLE 10.

FIG. 183 shows the nucleic acid coding sequence (SEQ ID NO: 202) for mannitol-1-phosphate 5-dehydrogenase, with gene designation of mtlD, as predicted from the genomic sequence of S. aureus. This predicted nucleic acid coding sequence was cloned and sequenced to produce the polynucleotide sequence shown in FIG. 185.

FIG. 184 shows the amino acid sequence (SEQ ID NO: 203) for mannitol-1-phosphate 5-dehydrogenase (mtlD) from S. aureus, as predicted from the nucleotide sequence SEQ ID NO: 202 shown in FIG. 183.

FIG. 185 shows the experimentally determined nucleic acid coding sequence (SEQ ID NO: 204) for mannitol-1-phosphate 5-dehydrogenase (mtlD) from S. aureus, as described in EXAMPLE 1.

FIG. 186 shows the amino acid sequence (SEQ ID NO: 205) for mannitol-1-phosphate 5-dehydrogenase (mtlD) from S. aureus, as predicted from the experimentally determined nucleotide sequence SEQ ID NO: 204 shown in FIG. 185.

FIG. 187 shows the primer sequences used to amplify the nucleic acid of SEQ ID NO: 204. The primers are SEQ ID NO: 206 and SEQ ID NO: 207.

FIG. 188 contains TABLE 45, which provides among other things a variety of data and other information on mannitol-1-phosphate 5-dehydrogenase (mtlD) from S. aureus.

FIG. 189 contains TABLE 46, which provides the results of several bioinformatic analyses relating to mannitol-1-phosphate 5-dehydrogenase (mtlD) from S. aureus.

FIG. 190 depicts the results of tryptic peptide mass spectrum peak searching for mannitol-1-phosphate 5-dehydrogenase (mtlD) from S. aureus, as described in EXAMPLE 9.

FIG. 191 shows the nucleic acid coding sequence (SEQ ID NO: 211) for dihydrofolate reductase, with gene designation of dfrA, as predicted from the genomic sequence of P. aeruginosa. This predicted nucleic acid coding sequence was cloned and sequenced to produce the polynucleotide sequence shown in FIG. 193.

FIG. 192 shows the amino acid sequence (SEQ ID NO: 212) for dihydrofolate reductase (dfrA) from P. aeruginosa, as predicted from the nucleotide sequence SEQ ID NO: 211 shown in FIG. 191.

FIG. 193 shows the experimentally determined nucleic acid coding sequence (SEQ ID NO: 213) for dihydrofolate reductase (dfrA) from P. aeruginosa, as described in EXAMPLE 1.

FIG. 194 shows the amino acid sequence (SEQ ID NO: 214) for dihydrofolate reductase (dfrA) from P. aeruginosa, as predicted from the experimentally determined nucleotide sequence SEQ ID NO: 213 shown in FIG. 193.

FIG. 195 shows the primer sequences used to amplify the nucleic acid of SEQ ID NO: 213. The primers are SEQ ID NO: 215 and SEQ ID NO: 216.

FIG. 196 contains TABLE 47, which provides among other things a variety of data and other information on dihydrofolate reductase (dfrA) from P. aeruginosa.

FIG. 197 contains TABLE 48, which provides the results of several bioinformatic analyses relating to dihydrofolate reductase (dfrA) from P. aeruginosa.

FIG. 198 depicts a ¹H, ¹⁵N Heteronuclear Single Quantum Coherence (HSQC) spectrum of dihydrofolate reductase (dfrA) from P. aeruginosa, as described in EXAMPLE 15 below. The X-axis shows a proton chemical shift, while the Y-axis shows the ¹⁵N chemical shift of the purified ¹⁵N labeled polypeptide.

FIG. 199 depicts the results of tryptic peptide mass spectrum peak searching for dihydrofolate reductase (dfrA) from P. aeruginosa, as described in EXAMPLE 9.

FIG. 200 depicts a MALDI-TOF mass spectrum of dihydrofolate reductase (dfrA) from P. aeruginosa, as described in EXAMPLE 10.

FIG. 201 shows the nucleic acid coding sequence (SEQ ID NO: 220) for dihydrofolate reductase, with gene designation of dfrA, as predicted from the genomic sequence of S. aureus. This predicted nucleic acid coding sequence was cloned and sequenced to produce the polynucleotide sequence shown in FIG. 203.

FIG. 202 shows the amino acid sequence (SEQ ID NO: 221) for dihydrofolate reductase (dfrA) from S. aureus, as predicted from the nucleotide sequence SEQ ID NO: 220 shown in 0.

FIG. 203 shows the experimentally determined nucleic acid coding sequence (SEQ ID NO: 222) for dihydrofolate reductase (dfrA) from S. aureus, as described in EXAMPLE 1.

FIG. 204 shows the amino acid sequence (SEQ ID NO: 223) for dihydrofolate reductase (dfrA) from S. aureus, as predicted from the experimentally determined nucleotide sequence SEQ ID NO: 222 shown in FIG. 203.

FIG. 205 shows the primer sequences used to amplify the nucleic acid of SEQ ID NO: 222. The primers are SEQ ID NO: 224 and SEQ ID NO: 225.

FIG. 206 contains TABLE 49, which provides among other things a variety of data and other information on dihydrofolate reductase (dfrA) from S. aureus.

FIG. 207 contains TABLE 50, which provides the results of several bioinformatic analyses relating to dihydrofolate reductase (dfrA) from S. aureus.

FIG. 208 depicts a ¹H, ¹⁵N Heteronuclear Single Quantum Coherence (HSQC) spectrum of dihydrofolate reductase (dfrA) from S. aureus, as described in EXAMPLE 15 below. The X-axis shows a proton chemical shift, while the Y-axis shows the ¹⁵N chemical shift of the purified ¹⁵N labeled polypeptide.

FIG. 209 shows the nucleic acid coding sequence (SEQ ID NO: 229) for replicative DNA helicase, with gene designation of dnaB, as predicted from the genomic sequence of S. aureus. This predicted nucleic acid coding sequence was cloned and sequenced to produce the polynucleotide sequence shown in FIG. 211.

FIG. 210 shows the amino acid sequence (SEQ ID NO: 230) for replicative DNA helicase (dnaB) from S. aureus, as predicted from the nucleotide sequence SEQ ID NO: 229 shown in FIG. 209.

FIG. 211 shows the experimentally determined nucleic acid coding sequence (SEQ ID NO: 231) for replicative DNA helicase (dnaB) from S. aureus, as described in EXAMPLE 1.

FIG. 212 shows the amino acid sequence (SEQ ID NO: 232) for replicative DNA helicase (dnaB) from S. aureus, as predicted from the experimentally determined nucleotide sequence SEQ ID NO: 231 shown in FIG. 211.

FIG. 213 shows the primer sequences used to amplify the nucleic acid of SEQ ID NO: 231. The primers are SEQ ID NO: 233 and SEQ ID NO: 234.

FIG. 214 contains TABLE 51, which provides among other things a variety of data and other information on replicative DNA helicase (dnaB) from S. aureus.

FIG. 215 contains TABLE 52, which provides the results of several bioinformatic analyses relating to replicative DNA helicase (dnaB) from S. aureus.

FIG. 216 depicts the results of tryptic peptide mass spectrum peak searching for replicative DNA helicase (dnaB) from S. aureus, as described in EXAMPLE 9.

FIG. 217 shows the nucleic acid coding sequence (SEQ ID NO: 238) for replicative DNA helicase, with gene designation of dnaB, as predicted from the genomic sequence of P. aeruginosa. This predicted nucleic acid coding sequence was cloned and sequenced to produce the polynucleotide sequence shown in FIG. 219.

FIG. 218 shows the amino acid sequence (SEQ ID NO: 239) for replicative DNA helicase (dnaB) from P. aeruginosa, as predicted from the nucleotide sequence SEQ ID NO: 238 shown in FIG. 217.

FIG. 219 shows the experimentally determined nucleic acid coding sequence (SEQ ID NO: 240) for replicative DNA helicase (dnaB) from P. aeruginosa, as described in EXAMPLE 1.

FIG. 220 shows the amino acid sequence (SEQ ID NO: 241) for replicative DNA helicase (dnaB) from P. aeruginosa, as predicted from the experimentally determined nucleotide sequence SEQ ID NO: 240 shown in FIG. 219.

FIG. 221 shows the primer sequences used to amplify the nucleic acid of SEQ ID NO: 240. The primers are SEQ ID NO: 242 and SEQ ID NO: 243.

FIG. 222 contains TABLE 53, which provides among other things a variety of data and other information on replicative DNA helicase (dnaB) from P. aeruginosa.

FIG. 223 contains TABLE 54, which provides the results of several bioinformatic analyses relating to replicative DNA helicase (dnaB) from P. aeruginosa.

FIG. 224 depicts the results of tryptic peptide mass spectrum peak searching for replicative DNA helicase (dnaB) from P. aeruginosa, as described in EXAMPLE 9.

FIG. 225 depicts a MALDI-TOF mass spectrum of replicative DNA helicase (dnaB) from P. aeruginosa, as described in EXAMPLE 10.

FIG. 226 shows the nucleic acid coding sequence (SEQ ID NO: 247) for cysteinyl-tRNA synthetase, with gene designation of cysS, as predicted from the genomic sequence of E. faecalis. This predicted nucleic acid coding sequence was cloned and sequenced to produce the polynucleotide sequence shown in FIG. 91.

FIG. 227 shows the amino acid sequence (SEQ ID NO: 248) for cysteinyl-tRNA synthetase (cysS) from E. faecalis, as predicted from the nucleotide sequence SEQ ID NO: 247 shown in FIG. 226.

FIG. 228 shows the experimentally determined nucleic acid coding sequence (SEQ ID NO: 249) for cysteinyl-tRNA synthetase (cysS) from E. faecalis, as described in EXAMPLE 1.

FIG. 229 shows the amino acid sequence (SEQ ID NO: 250) for cysteinyl-tRNA synthetase (cysS) from E. faecalis, as predicted from the experimentally determined nucleotide sequence SEQ ID NO: 105 shown in FIG. 91.

FIG. 230 shows the primer sequences used to amplify the nucleic acid of SEQ ID NO: 105. The primers are SEQ ID NO: 251 and SEQ ID NO: 252.

FIG. 231 contains TABLE 55, which provides among other things a variety of data and other information on cysteinyl-tRNA synthetase (cysS) from E. faecalis.

FIG. 232 contains TABLE 56, which provides the results of several bioinformatic analyses relating to cysteinyl-tRNA synthetase (cysS) from E. faecalis.

FIG. 233 depicts the results of tryptic peptide mass spectrum peak searching for cysteinyl-tRNA synthetase (cysS) from E. faecalis, as described in EXAMPLE 9.

FIG. 234 depicts a MALDI-TOF mass spectrum of cysteinyl-tRNA synthetase (cysS) from E. faecalis, as described in EXAMPLE 10.

FIG. 235 shows the nucleic acid coding sequence (SEQ ID NO: 256) for oxidoreductase, with gene designation of fabG, as predicted from the genomic sequence of E. faecalis. This predicted nucleic acid coding sequence was cloned and sequenced to produce the polynucleotide sequence shown in FIG. 237.

FIG. 236 shows the amino acid sequence (SEQ ID NO: 257) for oxidoreductase (fabG) from E. faecalis, as predicted from the nucleotide sequence SEQ ID NO: 256 shown in FIG. 235.

FIG. 237 shows the experimentally determined nucleic acid coding sequence (SEQ ID NO: 258) for oxidoreductase (fabG) from E. faecalis, as described in EXAMPLE 1.

FIG. 238 shows the amino acid sequence (SEQ ID NO: 259) for oxidoreductase (fabG) from E. faecalis, as predicted from the experimentally determined nucleotide sequence SEQ ID NO: 258 shown in FIG. 237.

FIG. 239 shows the primer sequences used to amplify the nucleic acid of SEQ ID NO: 258. The primers are SEQ ID NO: 260 and SEQ ID NO: 261.

FIG. 240 contains TABLE 57, which provides among other things a variety of data and other information on oxidoreductase (fabG) from E. faecalis.

FIG. 241 contains TABLE 58, which provides the results of several bioinformatic analyses relating to oxidoreductase (fabG) from E. faecalis.

FIG. 242 depicts the results of tryptic peptide mass spectrum peak searching for oxidoreductase (fabG) from E. faecalis, as described in EXAMPLE 9.

FIG. 243 depicts a MALDI-TOF mass spectrum of oxidoreductase (fabG) from E. faecalis, as described in EXAMPLE 10.

FIG. 244 shows the nucleic acid coding sequence (SEQ ID NO: 265) for cell division protein FtsA, with gene designation of ftsA, as predicted from the genomic sequence of E. faecalis. This predicted nucleic acid coding sequence was cloned and sequenced to produce the polynucleotide sequence shown in FIG. 91.

FIG. 245 shows the amino acid sequence (SEQ ID NO: 266) for cell division protein FtsA (ftsA) from E. faecalis, as predicted from the nucleotide sequence SEQ ID NO: 265 shown in FIG. 244.

FIG. 246 shows the experimentally determined nucleic acid coding sequence (SEQ ID NO: 267) for cell division protein FtsA (ftsA) from E. faecalis, as described in EXAMPLE 1.

FIG. 247 shows the amino acid sequence (SEQ ID NO: 268) for cell division protein FtsA (ftsA) from E. faecalis, as predicted from the experimentally determined nucleotide sequence SEQ ID NO: 267 shown in FIG. 246.

FIG. 248 shows the primer sequences used to amplify the nucleic acid of SEQ ID NO: 267. The primers are SEQ ID NO: 269 and SEQ ID NO: 270.

FIG. 249 contains TABLE 59, which provides among other things a variety of data and other information on cell division protein FtsA (ftsA) from E. faecalis.

FIG. 250 contains TABLE 60, which provides the results of several bioinformatic analyses relating to cell division protein FtsA (ftsA) from E. faecalis.

FIG. 251 depicts the results of tryptic peptide mass spectrum peak searching for cell division protein FtsA (ftsA) from E. faecalis, as described in EXAMPLE 9.

FIG. 252 depicts a MALDI-TOF mass spectrum of cell division protein FtsA (ftsA) from E. faecalis, as described in EXAMPLE 10.

FIG. 253 shows the nucleic acid coding sequence (SEQ ID NO: 274) for UDP-3-O-acyl N-acetylglucosamine deacetylase, with gene designation of lpxC, as predicted from the genomic sequence of H. pylori. This predicted nucleic acid coding sequence was cloned and sequenced to produce the polynucleotide sequence shown in FIG. 255.

FIG. 254 shows the amino acid sequence (SEQ ID NO: 275) for UDP-3-O-acyl N-acetylglucosamine deacetylase (lpxC) from H. pylori, as predicted from the nucleotide sequence SEQ ID NO: 274 shown in FIG. 253.

FIG. 255 shows the experimentally determined nucleic acid coding sequence (SEQ ID NO: 276) for UDP-3-O-acyl N-acetylglucosamine deacetylase (lpxC) from H. pylori, as described in EXAMPLE 1.

FIG. 256 shows the amino acid sequence (SEQ ID NO: 277) for UDP-3-O-acyl N-acetylglucosamine deacetylase (lpxC) from H. pylori, as predicted from the experimentally determined nucleotide sequence SEQ ID NO: 276 shown in FIG. 255.

FIG. 257 shows the primer sequences used to amplify the nucleic acid of SEQ ID NO: 276. The primers are SEQ ID NO: 278 and SEQ ID NO: 279.

FIG. 258 contains TABLE 61, which provides among other things a variety of data and other information on UDP-3-O-acyl N-acetylglucosamine deacetylase (lpxC) from H. pylori.

FIG. 259 contains TABLE 62, which provides the results of several bioinformatic analyses relating to UDP-3-O-acyl N-acetylglucosamine deacetylase (lpxC) from H. pylori.

FIG. 260 shows the nucleic acid coding sequence (SEQ ID NO: 283) for UDP-3-O-(3-hydroxymyristoyl)-glucosamine N-acyltransferase, with gene designation of lpxD, as predicted from the genomic sequence of E. coli. This predicted nucleic acid coding sequence was cloned and sequenced to produce the polynucleotide sequence shown in FIG. 262.

FIG. 261 shows the amino acid sequence (SEQ ID NO: 284) for UDP-3-O-(3-hydroxymyristoyl)-glucosamine N-acyltransferase (lpxD) from E. coli, as predicted from the nucleotide sequence SEQ ID NO: 283 shown in FIG. 260.

FIG. 262 shows the experimentally determined nucleic acid coding sequence (SEQ ID NO: 285) for UDP-3-O-(3-hydroxymyristoyl)-glucosamine N-acyltransferase (lpxD) from E. coli, as described in EXAMPLE 1.

FIG. 263 shows the amino acid sequence (SEQ ID NO: 286) for UDP-3-O-(3-hydroxymyristoyl)-glucosamine N-acyltransferase (lpxD) from E. coli, as predicted from the experimentally determined nucleotide sequence SEQ ID NO: 285 shown in FIG. 262.

FIG. 264 shows the primer sequences used to amplify the nucleic acid of SEQ ID NO: 285. The primers are SEQ ID NO: 287 and SEQ ID NO: 288.

FIG. 265 contains TABLE 63, which provides among other things a variety of data and other information on UDP-3-O-(3-hydroxymyristoyl)-glucosamine N-acyltransferase (lpxD) from E. coli.

FIG. 266 contains TABLE 64, which provides the results of several bioinformatic analyses relating to UDP-3-O-(3-hydroxymyristoyl)-glucosamine N-acyltransferase (lpxD) from E. coli.

FIG. 267 depicts the results of tryptic peptide mass spectrum peak searching for UDP-3-O-(3-hydroxymyristoyl)-glucosamine N-acyltransferase (lpxD) from E. coli, as described in EXAMPLE 9.

FIG. 268 depicts a MALDI-TOF mass spectrum of UDP-3-O-(3-hydroxymyristoyl)-glucosamine N-acyltransferase (lpxD) from E. coli, as described in EXAMPLE 10.

FIG. 269 shows the nucleic acid coding sequence (SEQ ID NO: 292) for glutamate racemase, with gene designation of murI, as predicted from the genomic sequence of E. coli. This predicted nucleic acid coding sequence was cloned and sequenced to produce the polynucleotide sequence shown in FIG. 271.

FIG. 270 shows the amino acid sequence (SEQ ID NO: 293) for glutamate racemase (murI) from E. coli, as predicted from the nucleotide sequence SEQ ID NO: 292 shown in FIG. 271.

FIG. 271 shows the experimentally determined nucleic acid coding sequence (SEQ ID NO: 294) for glutamate racemase (murI) from E. coli, as described in EXAMPLE 1.

FIG. 272 shows the amino acid sequence (SEQ ID NO: 295) for glutamate racemase (murI) from E. coli, as predicted from the experimentally determined nucleotide sequence SEQ ID NO: 294 shown in FIG. 271.

FIG. 273 shows the primer sequences used to amplify the nucleic acid of SEQ ID NO: 294. The primers are SEQ ID NO: 296 and SEQ ID NO: 297.

FIG. 274 contains TABLE 65, which provides among other things a variety of data and other information on glutamate racemase (murI) from E. coli.

FIG. 275 contains TABLE 66, which provides the results of several bioinformatic analyses relating to glutamate racemase (murI) from E. coli.

FIG. 276 depicts the results of tryptic peptide mass spectrum peak searching for glutamate racemase (murI) from E. coli, as described in EXAMPLE 9.

FIG. 277 depicts a MALDI-TOF mass spectrum of glutamate racemase (murI) from E. coli, as described in EXAMPLE 10.

FIG. 278 shows the nucleic acid coding sequence (SEQ ID NO: 301) for N utilization substance protein A, with gene designation of nusA, as predicted from the genomic sequence of E. faecalis. This predicted nucleic acid coding sequence was cloned and sequenced to produce the polynucleotide sequence shown in FIG. 91.

FIG. 279 shows the amino acid sequence (SEQ ID NO: 302) for N utilization substance protein A (nusA) from E. faecalis, as predicted from the nucleotide sequence SEQ ID NO: 301 shown in FIG. 278.

FIG. 280 shows the experimentally determined nucleic acid coding sequence (SEQ ID NO: 303) for N utilization substance protein A (nusA) from E. faecalis, as described in EXAMPLE 1.

FIG. 281 shows the amino acid sequence (SEQ ID NO: 304) for N utilization substance protein A (nusA) from E. faecalis, as predicted from the experimentally determined nucleotide sequence SEQ ID NO: 303 shown in FIG. 280.

FIG. 282 shows the primer sequences used to amplify the nucleic acid of SEQ ID NO: 303. The primers are SEQ ID NO: 305 and SEQ ID NO: 306.

FIG. 283 contains TABLE 67, which provides among other things a variety of data and other information on N utilization substance protein A (nusA) from E. faecalis.

FIG. 284 contains TABLE 68, which provides the results of several bioinformatic analyses relating to N utilization substance protein A (nusA) from E. faecalis.

FIG. 285 depicts the results of tryptic peptide mass spectrum peak searching for N utilization substance protein A (nusA) from E. faecalis, as described in EXAMPLE 9.

FIG. 286 depicts a MALDI-TOF mass spectrum of N utilization substance protein A (nusA) from E. faecalis, as described in EXAMPLE 10.

FIG. 287 shows the nucleic acid coding sequence (SEQ ID NO: 310) for transcription termination factor NusA, with gene designation of nusA, as predicted from the genomic sequence of H. pylori. This predicted nucleic acid coding sequence was cloned and sequenced to produce the polynucleotide sequence shown in FIG. 289.

FIG. 288 shows the amino acid sequence (SEQ ID NO: 311) for transcription termination factor NusA (nusA) from H. pylori, as predicted from the nucleotide sequence SEQ ID NO: 310 shown in FIG. 287.

FIG. 289 shows the experimentally determined nucleic acid coding sequence (SEQ ID NO: 312) for transcription termination factor NusA (nusA) from H. pylori, as described in EXAMPLE 1.

FIG. 290 shows the amino acid sequence (SEQ ID NO: 313) for transcription termination factor NusA (nusA) from H. pylori, as predicted from the experimentally determined nucleotide sequence SEQ ID NO: 312 shown in FIG. 289.

FIG. 291 shows the primer sequences used to amplify the nucleic acid of SEQ ID NO: 312. The primers are SEQ ID NO: 314 and SEQ ID NO: 315.

FIG. 292 contains TABLE 69, which provides among other things a variety of data and other information on transcription termination factor NusA (nusA) from H. pylori.

FIG. 293 contains TABLE 70, which provides the results of several bioinformatic analyses relating to transcription termination factor NusA (nusA) from H. pylori.

FIG. 294 shows the nucleic acid coding sequence (SEQ ID NO: 319) for transcription termination factor, with gene designation of nusB, as predicted from the genomic sequence of H. pylori. This predicted nucleic acid coding sequence was cloned and sequenced to produce the polynucleotide sequence shown in FIG. 91.

FIG. 295 shows the amino acid sequence (SEQ ID NO: 320) for transcription termination factor (nusB) from H. pylori, as predicted from the nucleotide sequence SEQ ID NO: 319 shown in FIG. 294.

FIG. 296 shows the experimentally determined nucleic acid coding sequence (SEQ ID NO: 321) for transcription termination factor (nusB) from H. pylori, as described in EXAMPLE 1.

FIG. 297 shows the amino acid sequence (SEQ ID NO: 322) for transcription termination factor (nusB) from H. pylori, as predicted from the experimentally determined nucleotide sequence SEQ ID NO: 321 shown in FIG. 296.

FIG. 298 shows the primer sequences used to amplify the nucleic acid of SEQ ID NO: 321. The primers are SEQ ID NO: 323 and SEQ ID NO: 324.

FIG. 299 contains TABLE 71, which provides among other things a variety of data and other information on transcription termination factor (nusB) from H. pylori.

FIG. 300 contains TABLE 72, which provides the results of several bioinformatic analyses relating to transcription termination factor (nusB) from H. pylori.

FIG. 301 depicts a ¹H, ¹⁵N Heteronuclear Single Quantum Coherence (HSQC) spectrum of transcription termination factor (nusB) from H. pylori, as described in EXAMPLE 15 below. The X-axis shows a proton chemical shift, while the Y-axis shows the ¹⁵N chemical shift of the purified ¹⁵N labeled polypeptide.

FIG. 302 shows the nucleic acid coding sequence (SEQ ID NO: 328) for transcription termination factor, with gene designation of nusB, as predicted from the genomic sequence of S. aureus. This predicted nucleic acid coding sequence was cloned and sequenced to produce the polynucleotide sequence shown in FIG. 304.

FIG. 303 shows the amino acid sequence (SEQ ID NO: 329) for transcription termination factor (nusB) from S. aureus, as predicted from the nucleotide sequence SEQ ID NO: 328 shown in FIG. 302.

FIG. 304 shows the experimentally determined nucleic acid coding sequence (SEQ ID NO: 330) for transcription termination factor (nusB) from S. aureus, as described in EXAMPLE 1.

FIG. 305 shows the amino acid sequence (SEQ ID NO: 331) for transcription termination factor (nusB) from S. aureus, as predicted from the experimentally determined nucleotide sequence FIG. 304 shown in FIG. 304.

FIG. 306 shows the primer sequences used to amplify the nucleic acid of FIG. 304. The primers are SEQ ID NO: 332 and SEQ ID NO: 333.

FIG. 307 contains TABLE 73, which provides among other things a variety of data and other information on transcription termination factor (nusB) from S. aureus.

FIG. 308 contains TABLE 74, which provides the results of several bioinformatic analyses relating to transcription termination factor (nusB) from S. aureus.

FIG. 309 depicts a ¹H, ¹⁵N Heteronuclear Single Quantum Coherence (HSQC) spectrum of transcription termination factor (nusB) from S. aureus, as described in EXAMPLE 15 below. The X-axis shows a proton chemical shift, while the Y-axis shows the ¹⁵N chemical shift of the purified ¹⁵N labeled polypeptide.

FIG. 310 depicts the results of tryptic peptide mass spectrum peak searching for transcription termination factor (nusB) from S. aureus, as described in EXAMPLE 9.

FIG. 311 shows the nucleic acid coding sequence (SEQ ID NO: 337) for ATP-dependent DNA helicase, with gene designation of pcrA, as predicted from the genomic sequence of S. pneumoniae. This predicted nucleic acid coding sequence was cloned and sequenced to produce the polynucleotide sequence shown in FIG. 313.

FIG. 312 shows the amino acid sequence (SEQ ID NO: 338) for ATP-dependent DNA helicase (pcrA) from S. pneumoniae, as predicted from the nucleotide sequence SEQ ID NO: 337 shown in FIG. 311.

FIG. 313 shows the experimentally determined nucleic acid coding sequence (SEQ ID NO: 339) for ATP-dependent DNA helicase (pcrA) from S. pneumoniae, as described in EXAMPLE 1.

FIG. 314 shows the amino acid sequence (SEQ ID NO: 340) for ATP-dependent DNA helicase (pcrS) from S. pneumoniae, as predicted from the experimentally determined nucleotide sequence SEQ ID NO: 339 shown in FIG. 313.

FIG. 315 shows the primer sequences used to amplify the nucleic acid of SEQ ID NO: 339. The primers are SEQ ID NO: 341 and SEQ ID NO: 342.

FIG. 316 contains TABLE 75, which provides among other things a variety of data and other information on ATP-dependent DNA helicase (pcrA) from S. pneumoniae.

FIG. 317 contains TABLE 76, which provides the results of several bioinformatic analyses relating to ATP-dependent DNA helicase (pcrA) from S. pneumoniae.

FIG. 318 depicts the results of tryptic peptide mass spectrum peak searching for ATP-dependent DNA helicase (pcrA) from S. pneumoniae, as described in EXAMPLE 9.

FIG. 319 depicts a MALDI-TOF mass spectrum of ATP-dependent DNA helicase (pcrA) from S. pneumoniae, as described in EXAMPLE 10.

FIG. 320 shows the nucleic acid coding sequence (SEQ ID NO: 346) for DNA-directed RNA polymerase, alpha subunit, with gene designation of rpoA, as predicted from the genomic sequence of E. faecalis. This predicted nucleic acid coding sequence was cloned and sequenced to produce the polynucleotide sequence shown in FIG. 322.

FIG. 321 shows the amino acid sequence (SEQ ID NO: 347) for DNA-directed RNA polymerase, alpha subunit (rpoA) from E. faecalis, as predicted from the nucleotide sequence SEQ ID NO: 346 shown in FIG. 320.

FIG. 322 shows the experimentally determined nucleic acid coding sequence (SEQ ID NO: 348) for DNA-directed RNA polymerase, alpha subunit (rpoA) from E. faecalis, as described in EXAMPLE 1.

FIG. 323 shows the amino acid sequence (SEQ ID NO: 349) for DNA-directed RNA polymerase, alpha subunit (rpoA) from E. faecalis, as predicted from the experimentally determined nucleotide sequence SEQ ID NO: 348 shown in FIG. 322.

FIG. 324 shows the primer sequences used to amplify the nucleic acid of SEQ ID NO: 348. The primers are SEQ ID NO: 350 and SEQ ID NO: 351.

FIG. 325 contains TABLE 77, which provides among other things a variety of data and other information on DNA-directed RNA polymerase, alpha subunit (rpoA) from E. faecalis.

FIG. 326 contains TABLE 78, which provides the results of several bioinformatic analyses relating to DNA-directed RNA polymerase, alpha subunit (rpoA) from E. faecalis.

FIG. 327 depicts the results of tryptic peptide mass spectrum peak searching for DNA-directed RNA polymerase, alpha subunit (rpoA) from E. faecalis, as described in EXAMPLE 9.

FIG. 328 depicts a MALDI-TOF mass spectrum of DNA-directed RNA polymerase, alpha subunit (boa) from E. faecalis, as described in EXAMPLE 10.

FIG. 329 shows the nucleic acid coding sequence (SEQ ID NO: 355) for RNA polymerase sigma-70 factor family protein, with gene designation of rpoD, as predicted from the genomic sequence of E. faecalis. This predicted nucleic acid coding sequence was cloned and sequenced to produce the polynucleotide sequence shown in FIG. 331.

FIG. 330 shows the amino acid sequence (SEQ ID NO: 356) for RNA polymerase sigma-70 factor family protein (rpoD) from E. faecalis, as predicted from the nucleotide sequence SEQ ID NO: 355 shown in FIG. 329.

FIG. 331 shows the experimentally determined nucleic acid coding sequence (SEQ ID NO: 357) for RNA polymerase sigma-70 factor family protein (rpoD) from E. faecalis, as described in EXAMPLE 1.

FIG. 332 shows the amino acid sequence (SEQ ID NO: 358) for RNA polymerase sigma-70 factor family protein (rpoD) from E. faecalis, as predicted from the experimentally determined nucleotide sequence SEQ ID NO: 357 shown in FIG. 331.

FIG. 333 shows the primer sequences used to amplify the nucleic acid of SEQ ID NO: 357. The primers are SEQ ID NO: 359 and SEQ ID NO: 360.

FIG. 334 contains TABLE 79, which provides among other things a variety of data and other information on RNA polymerase sigma-70 factor family protein (rpoD) from E. faecalis.

FIG. 335 contains TABLE 80, which provides the results of several bioinformatic analyses relating to RNA polymerase sigma-70 factor family protein (rpoD) from E. faecalis.

FIG. 336 depicts the results of tryptic peptide mass spectrum peak searching for RNA polymerase sigma-70 factor family protein (rpoD) from E. faecalis, as described in EXAMPLE 9.

FIG. 337 depicts a MALDI-TOF mass spectrum of RNA polymerase sigma-70 factor family protein (rpoD) from E. faecalis, as described in EXAMPLE 10.

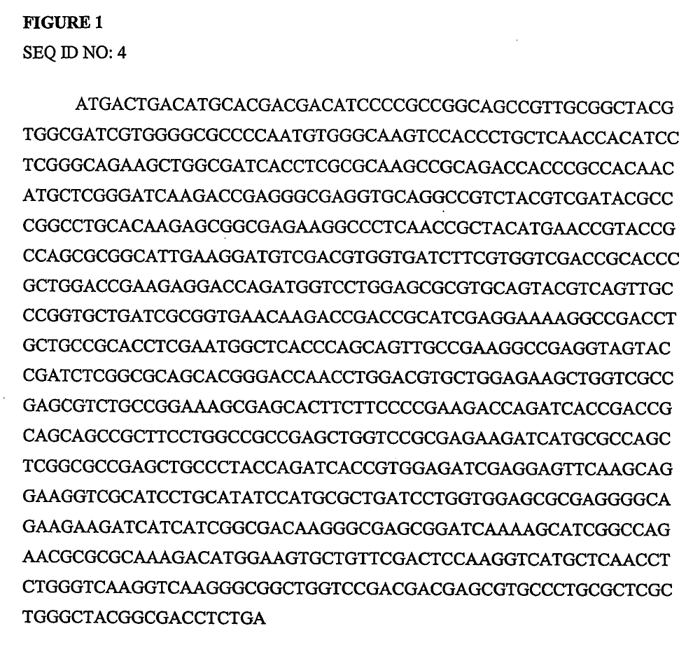

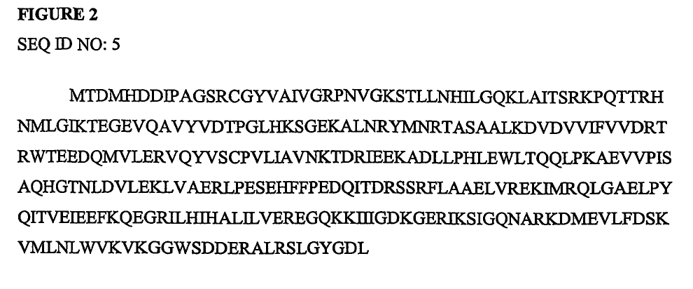

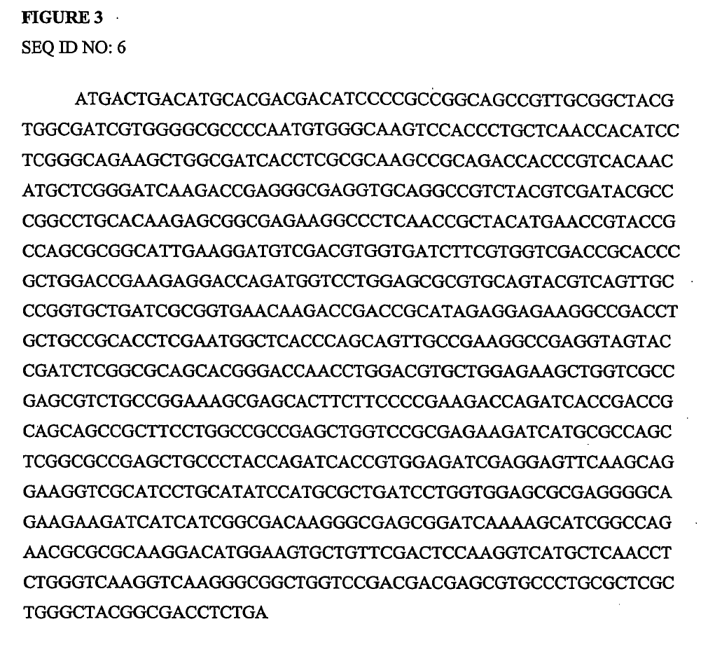

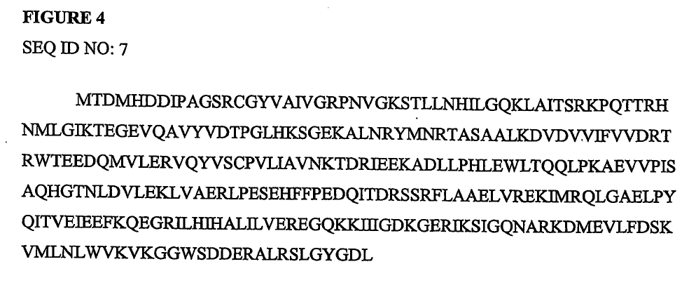

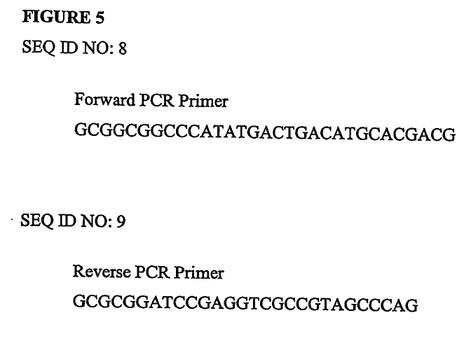

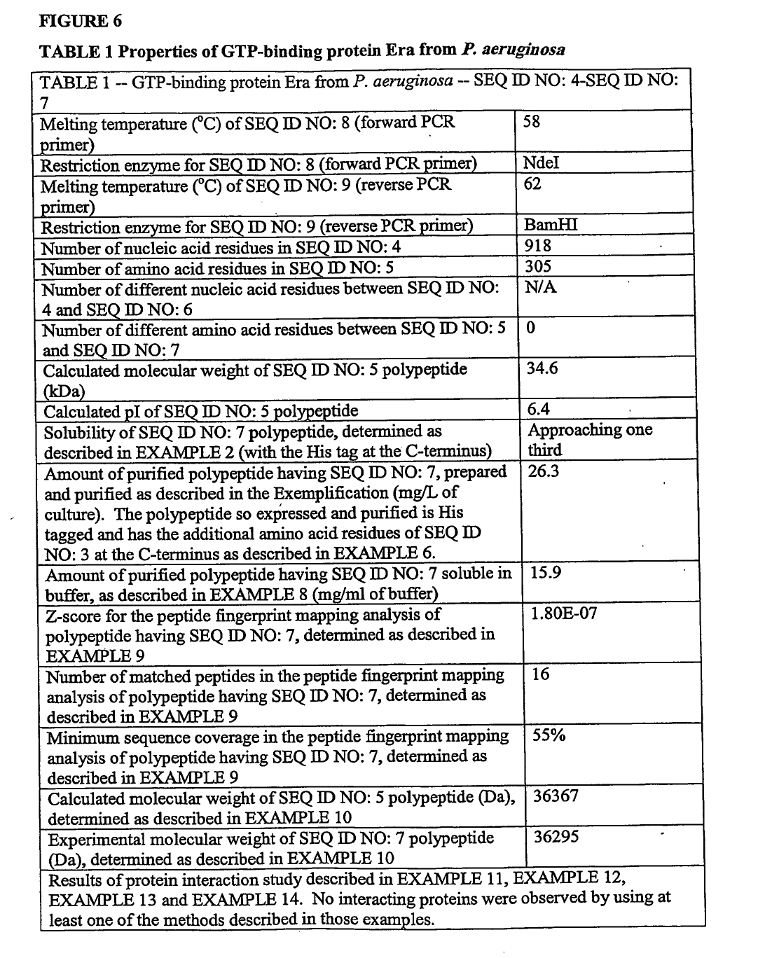

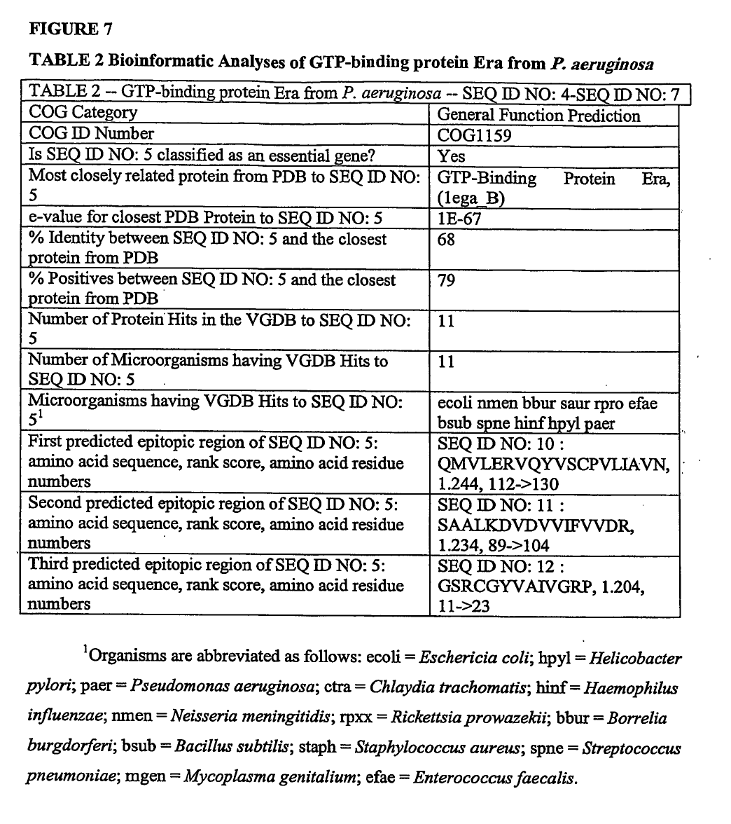

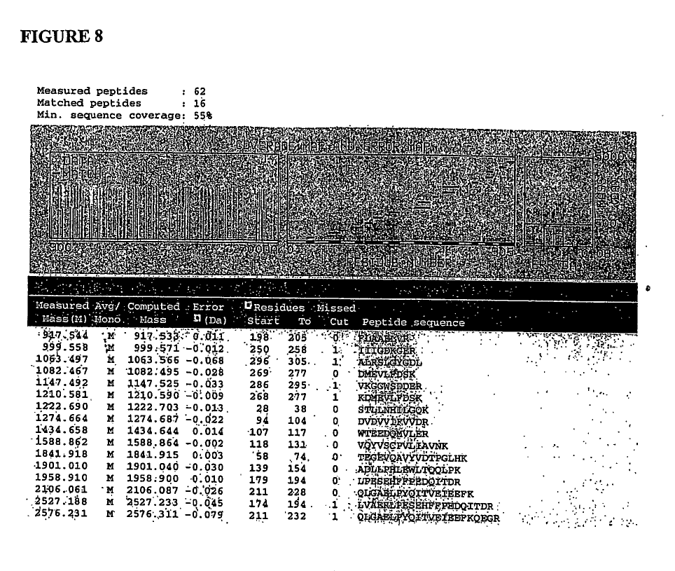

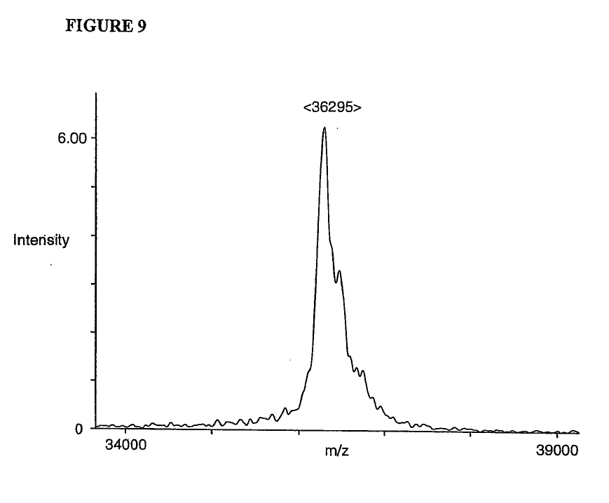

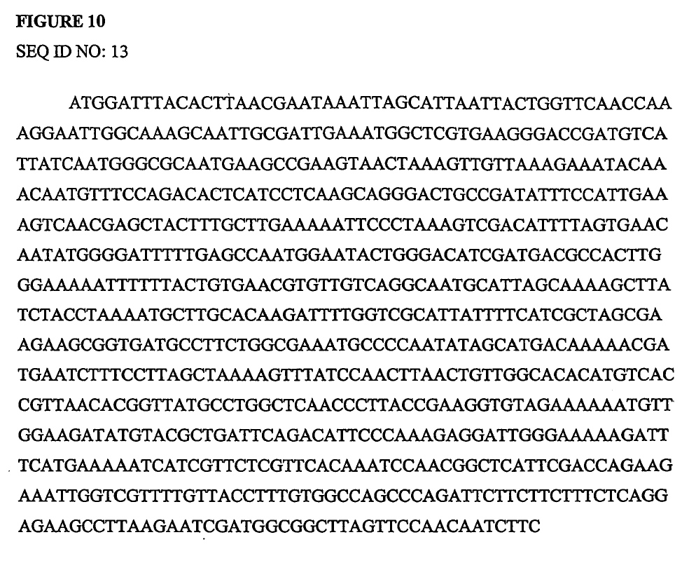

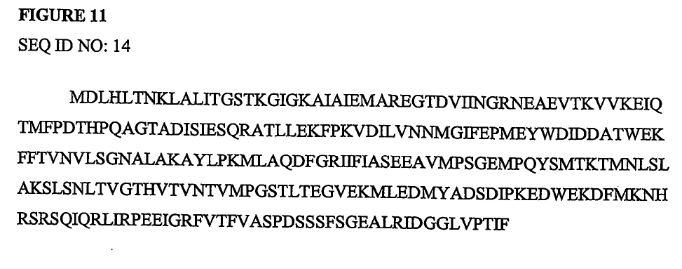

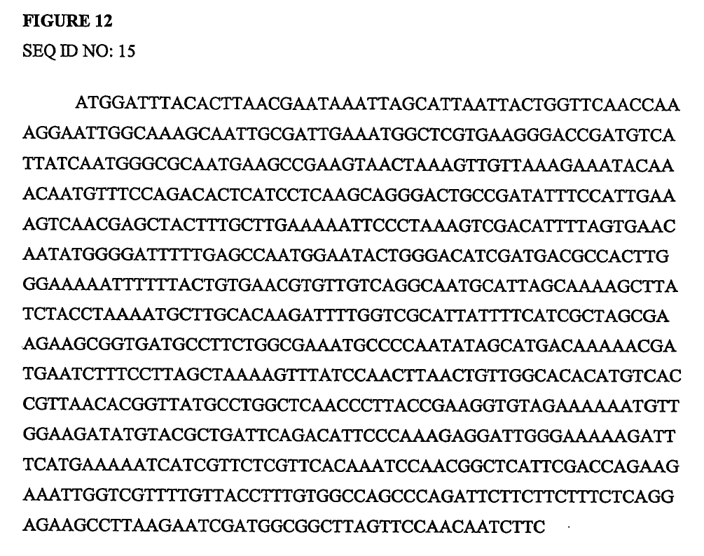

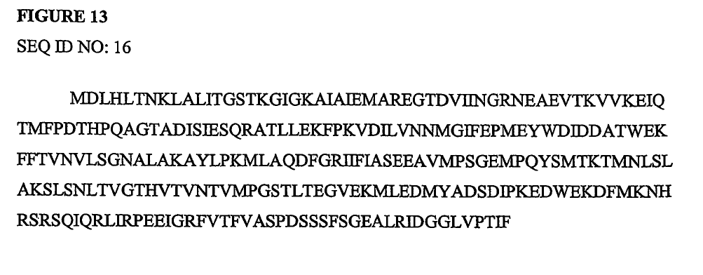

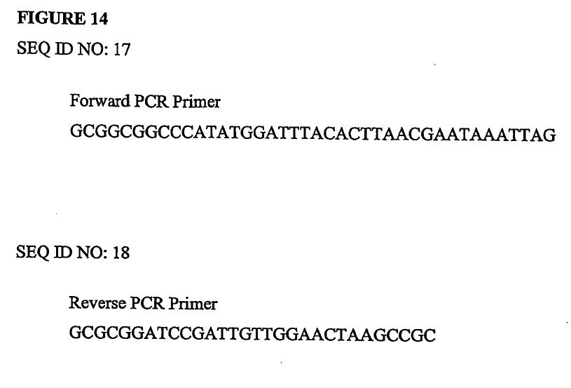

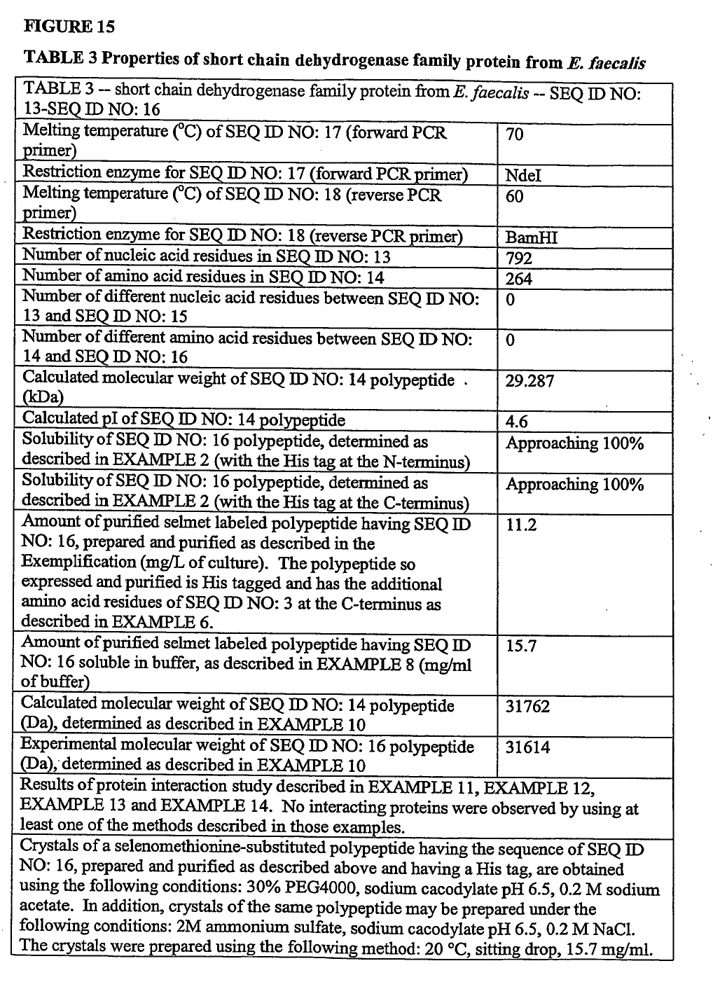

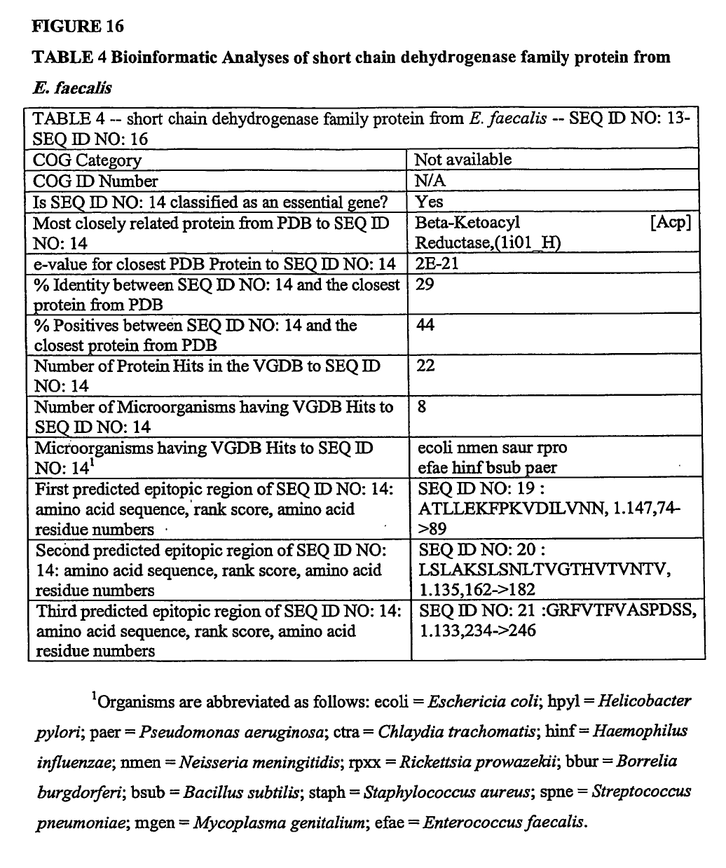

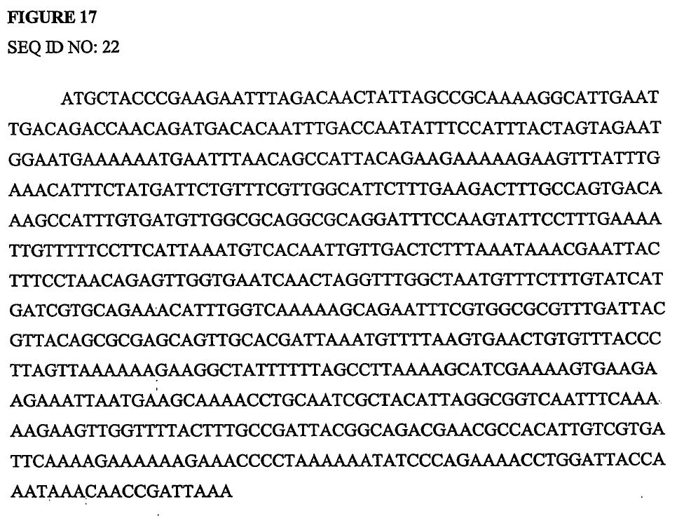

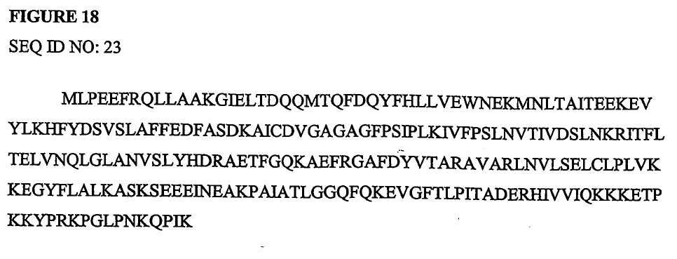

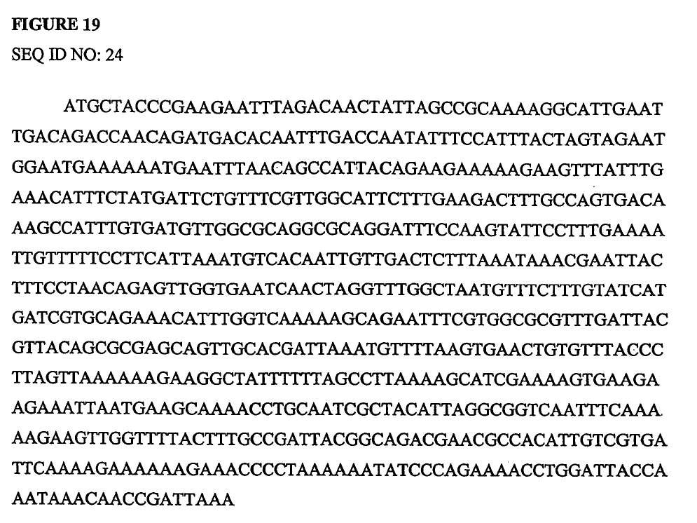

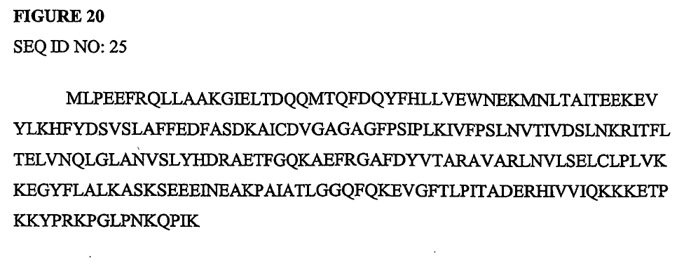

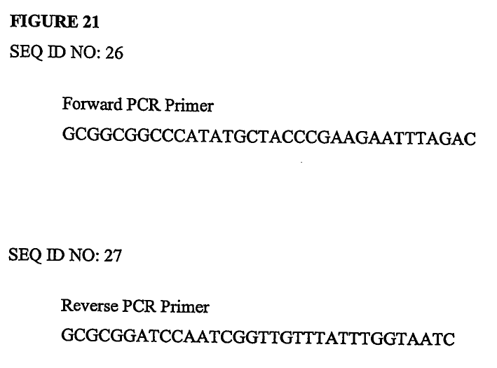

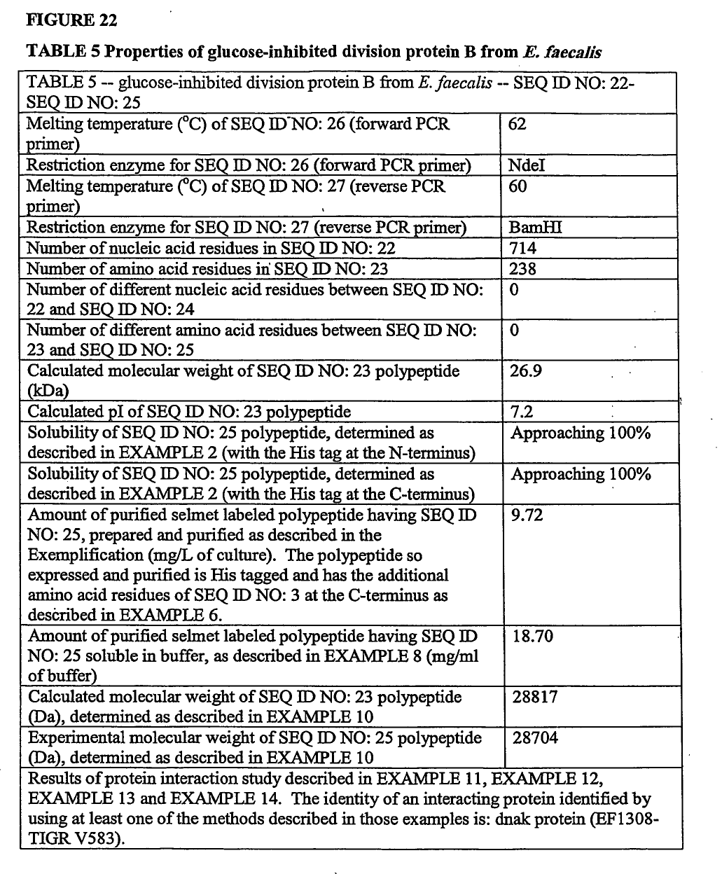

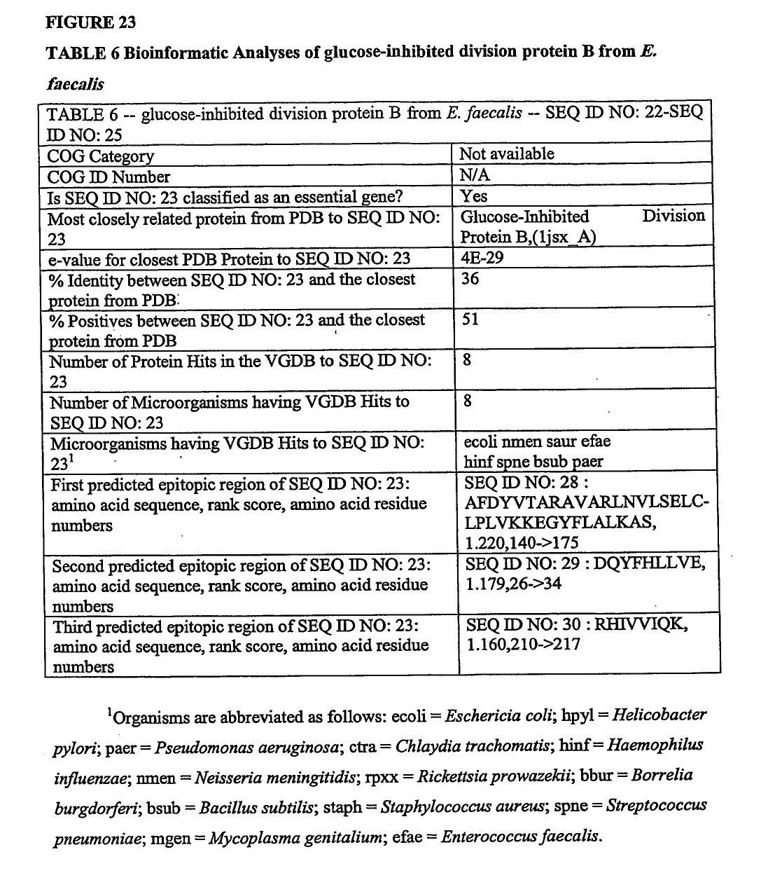

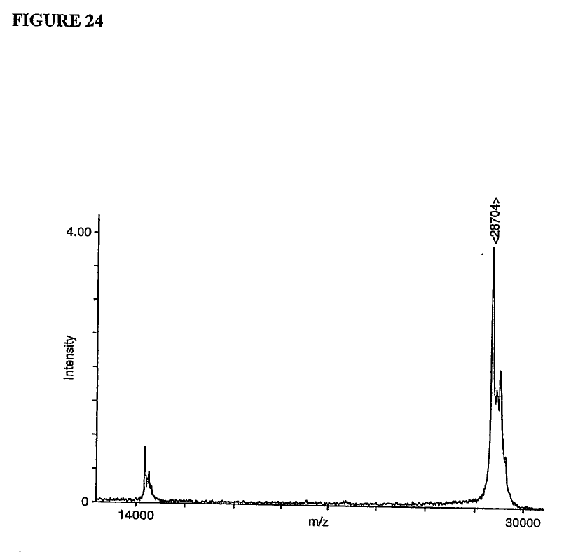

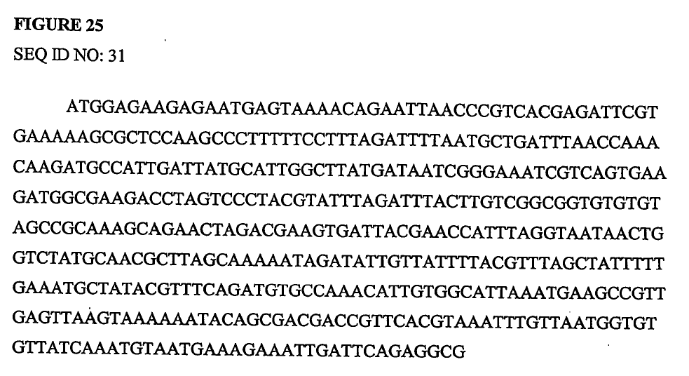

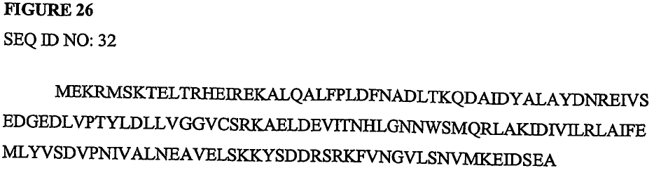

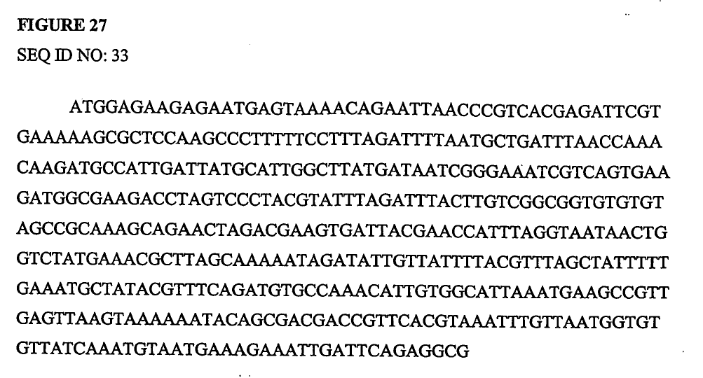

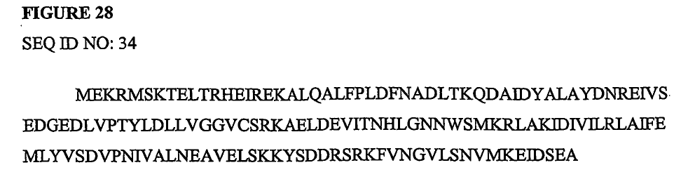

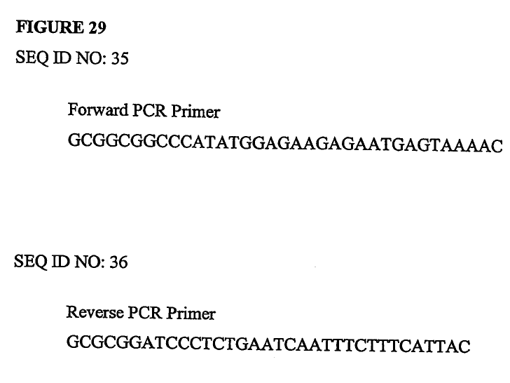

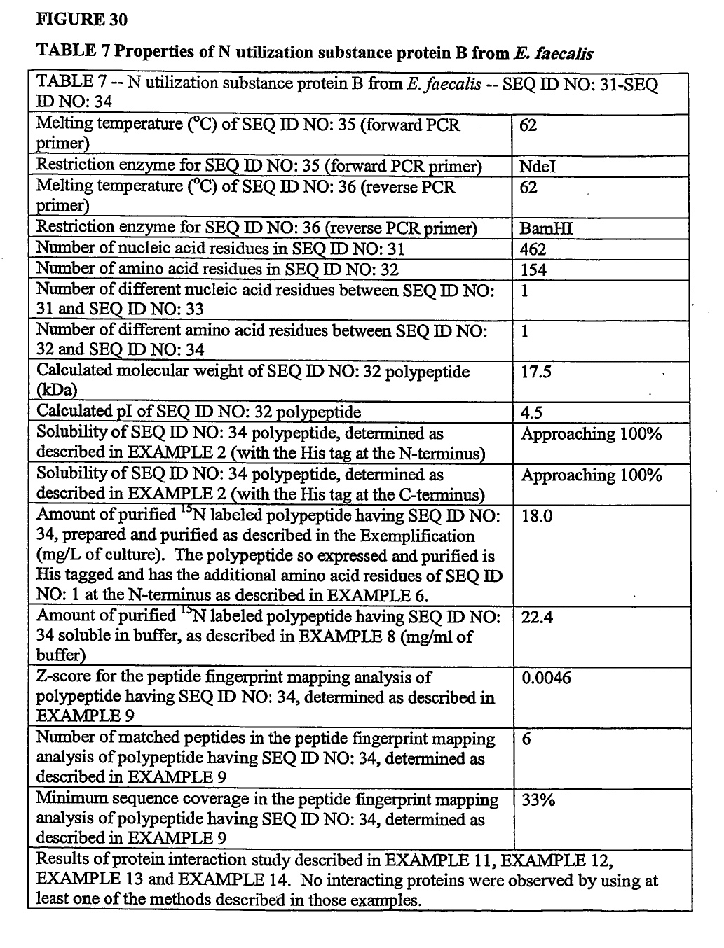

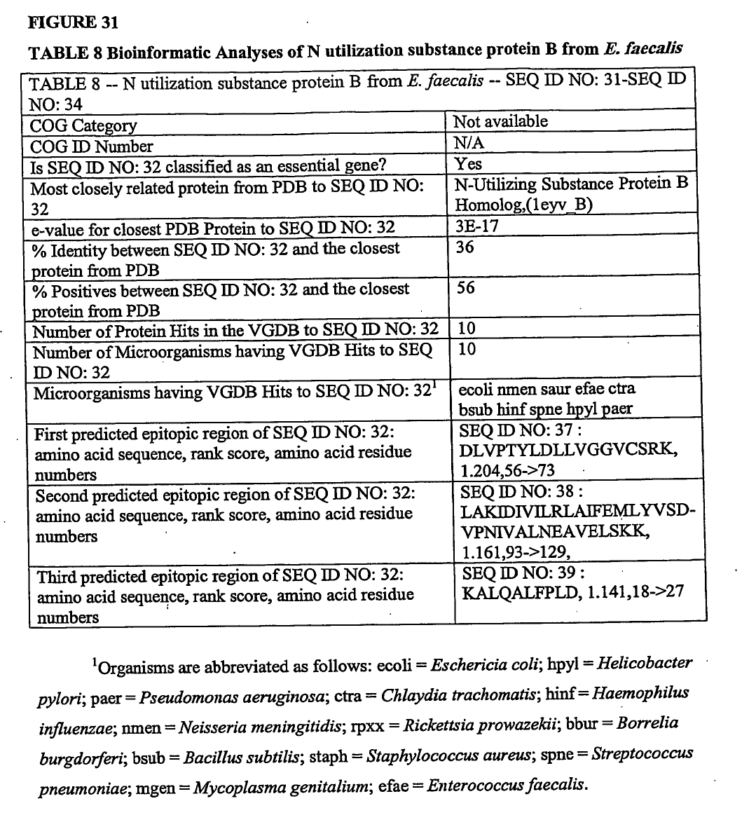

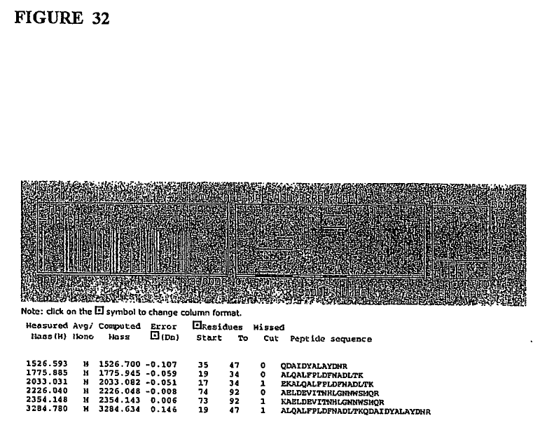

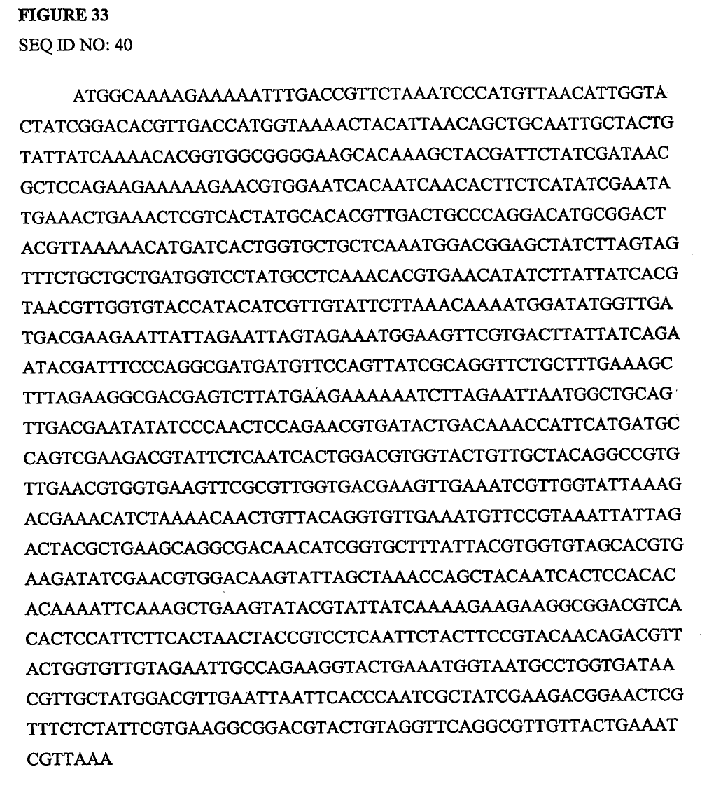

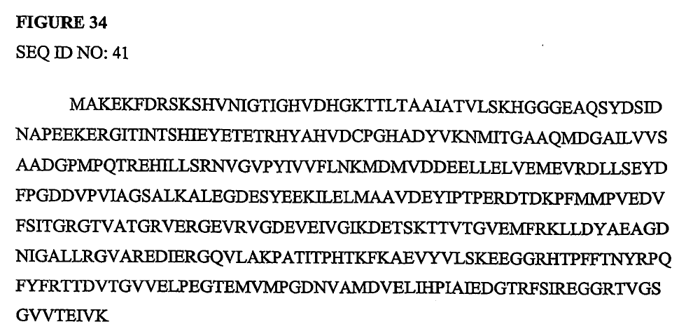

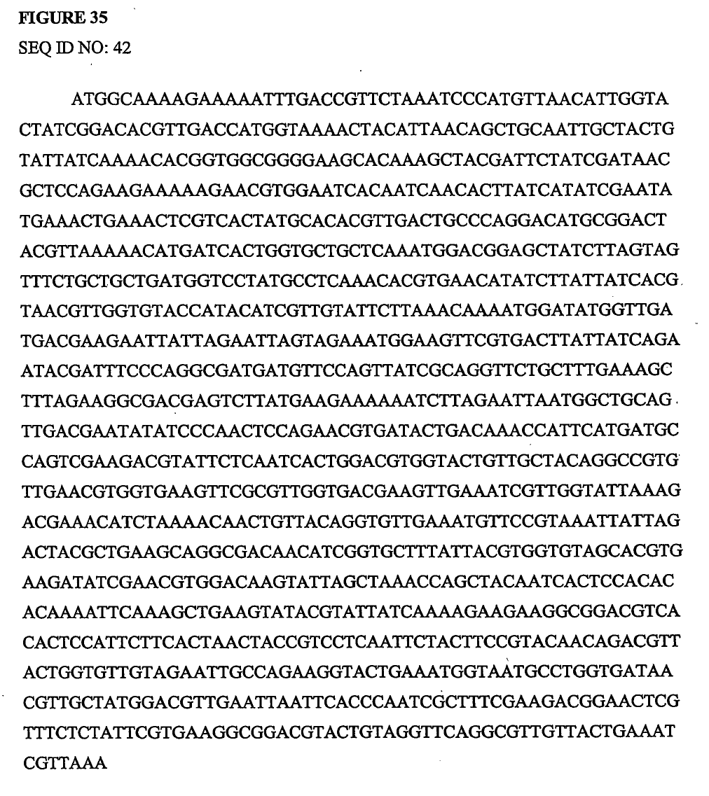

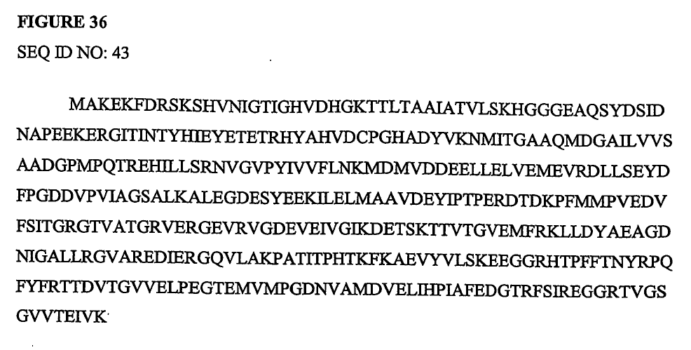

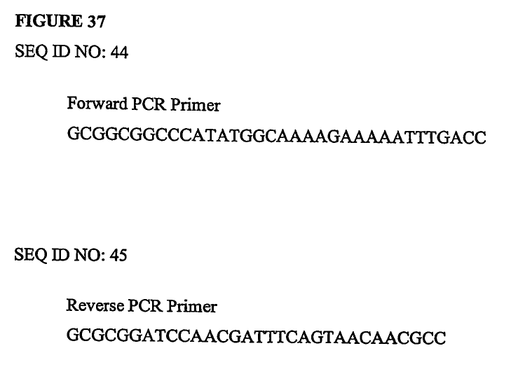

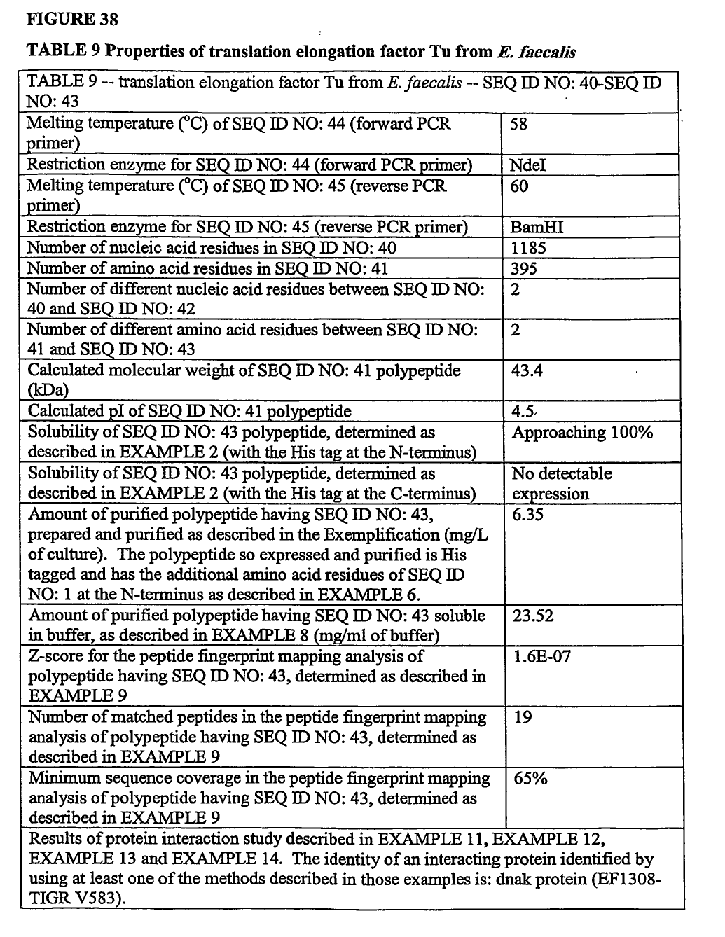

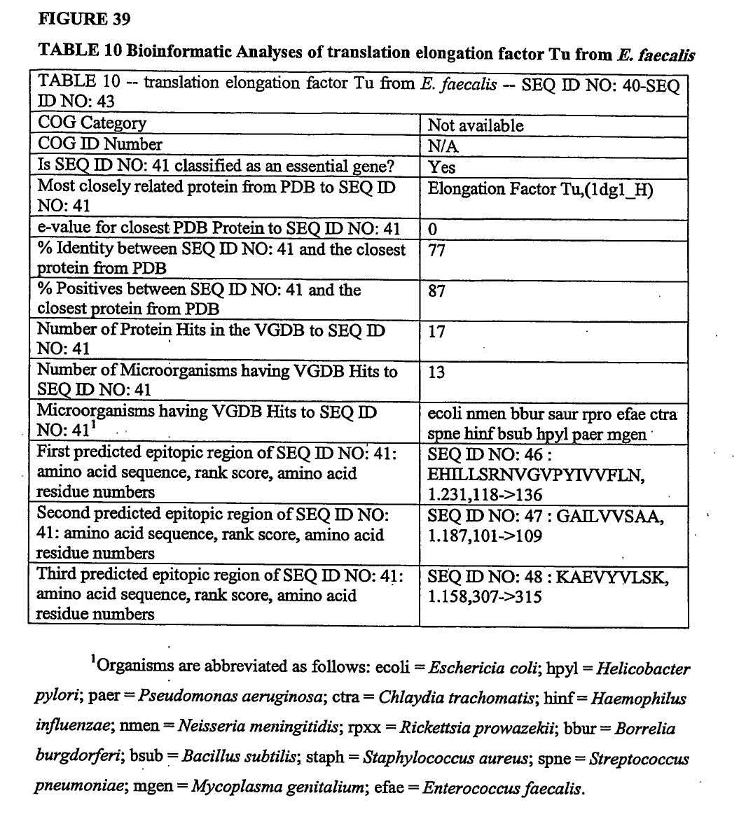

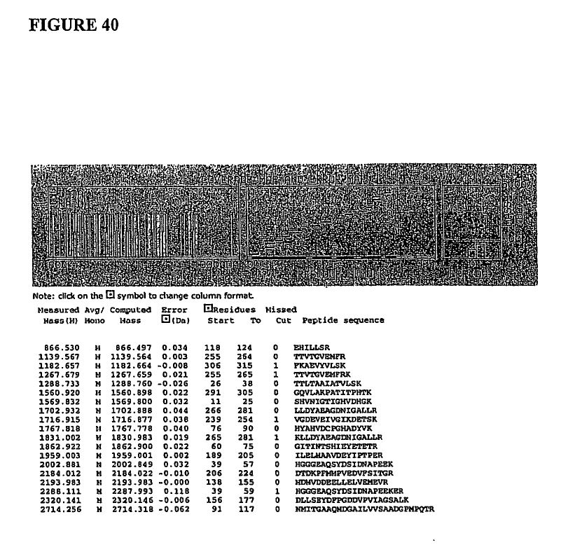

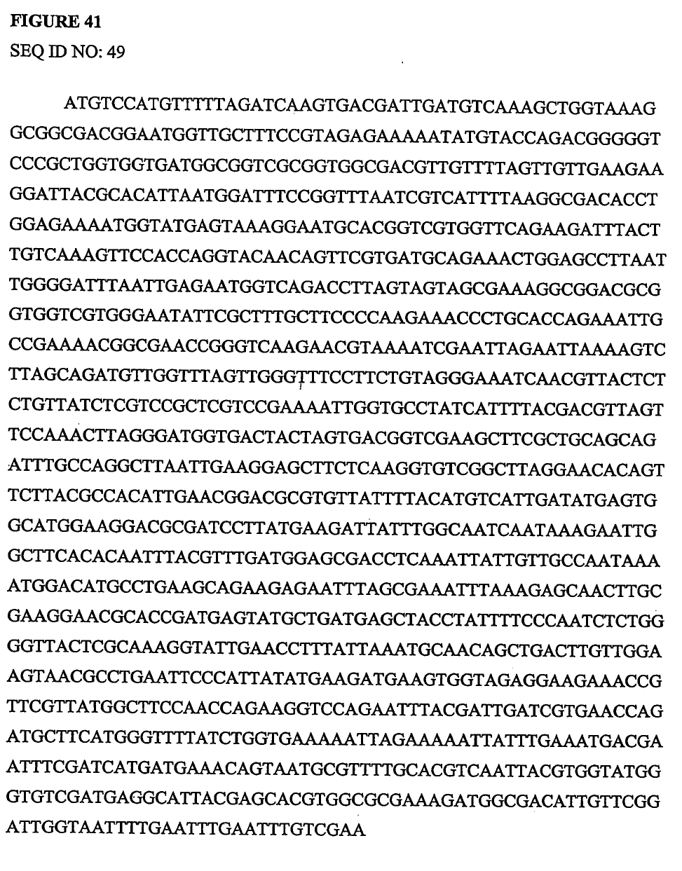

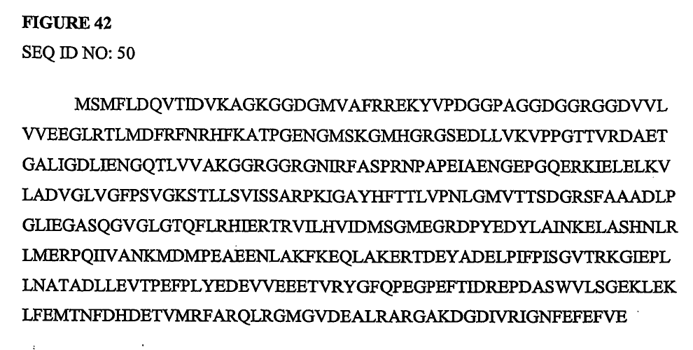

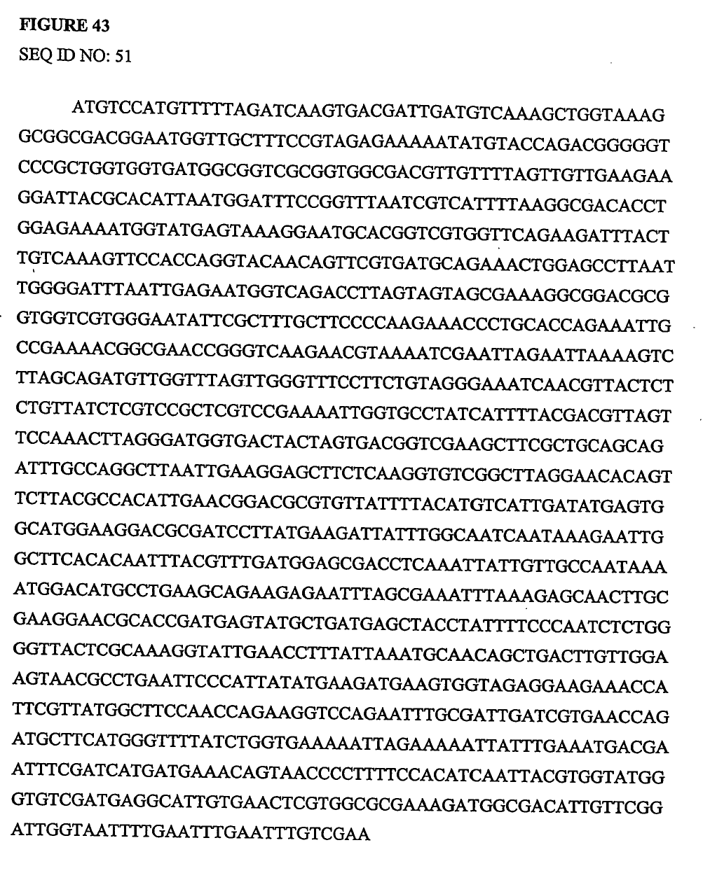

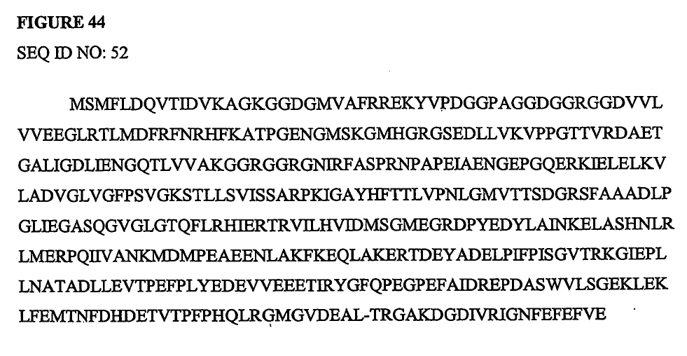

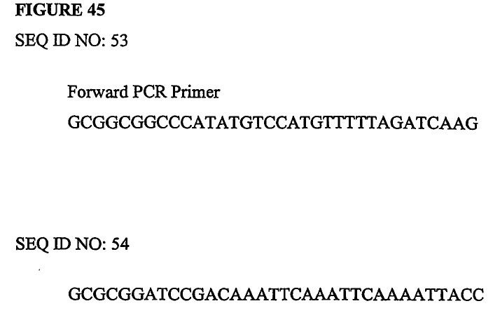

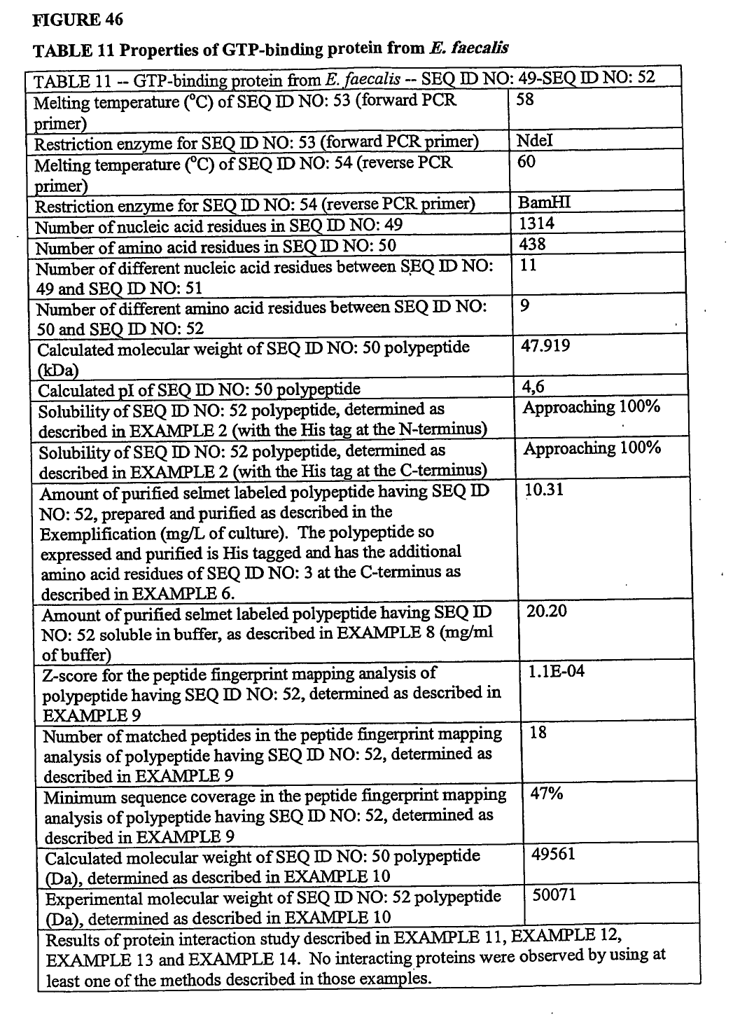

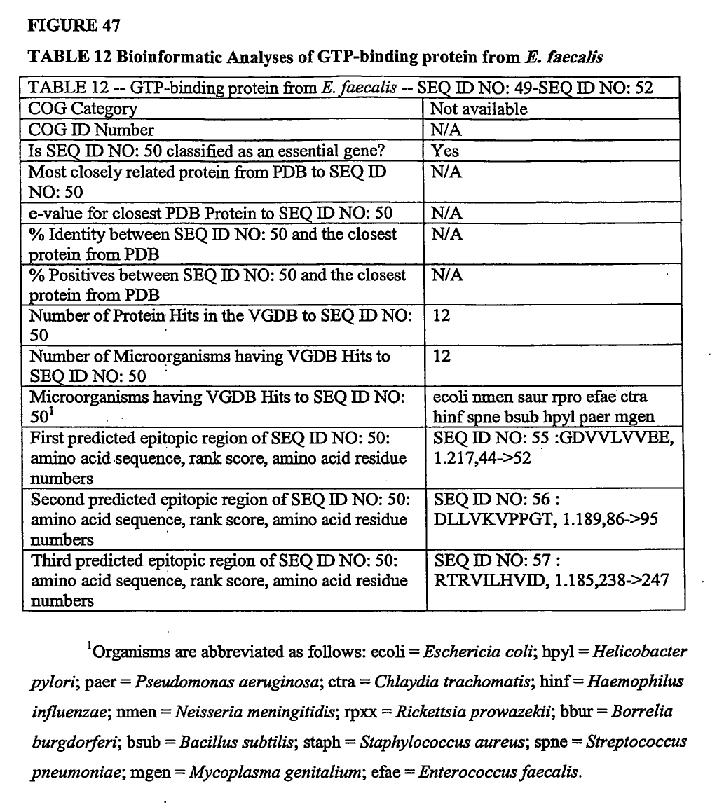

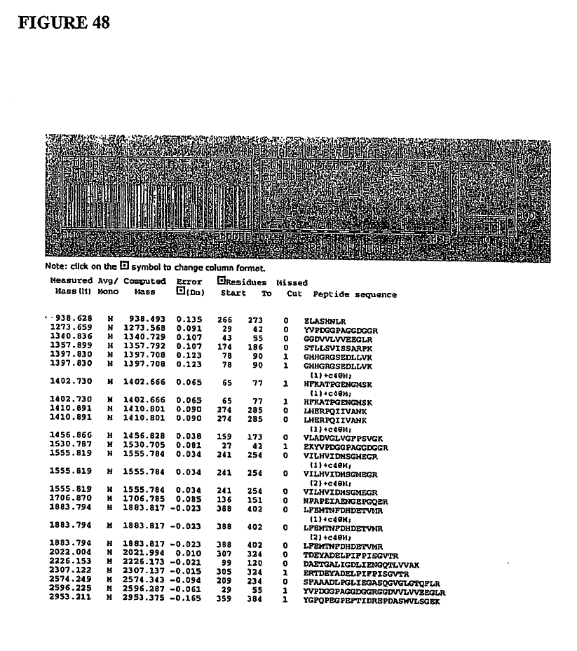

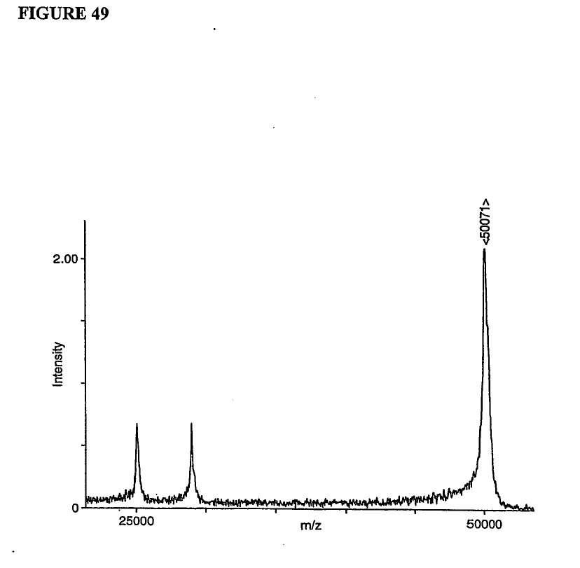

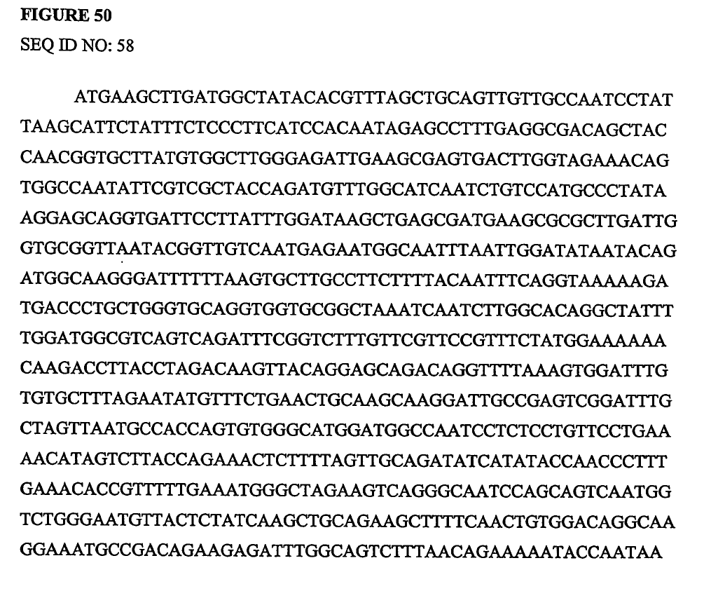

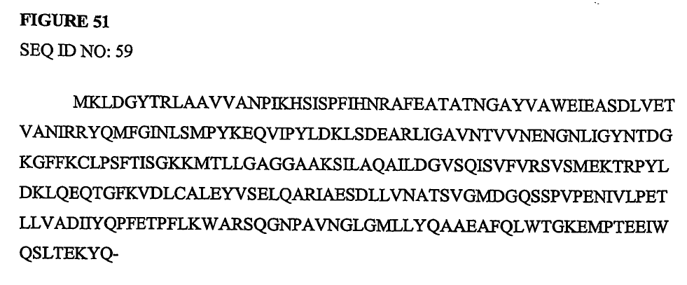

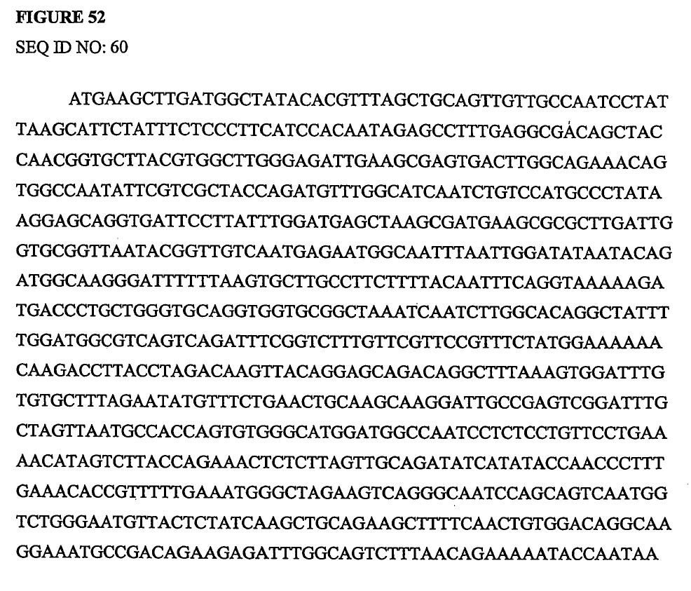

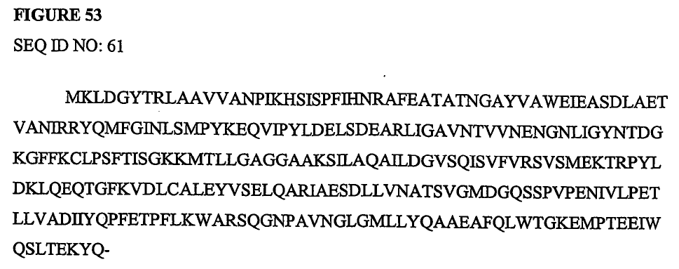

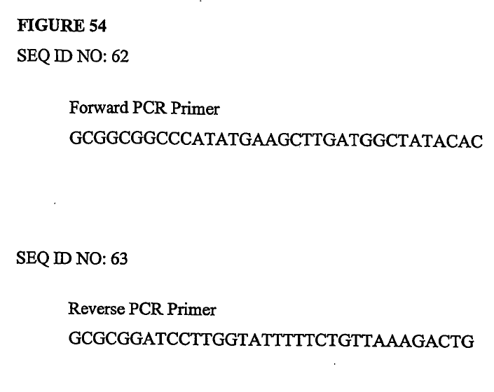

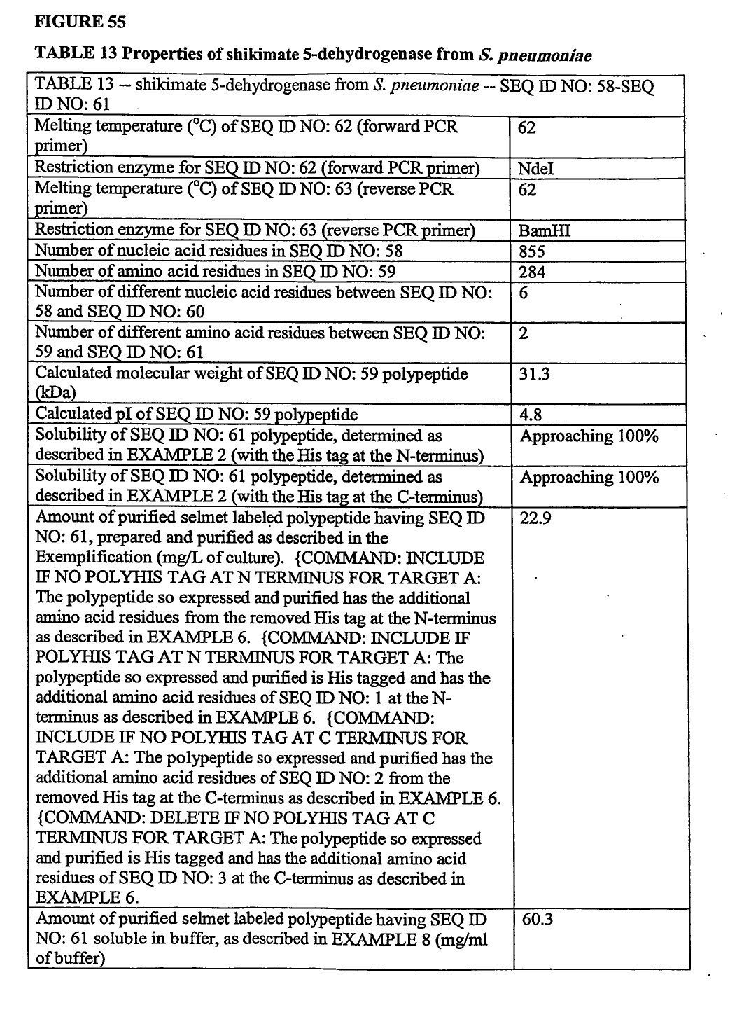

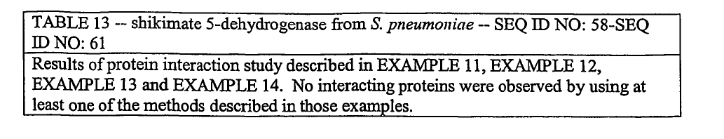

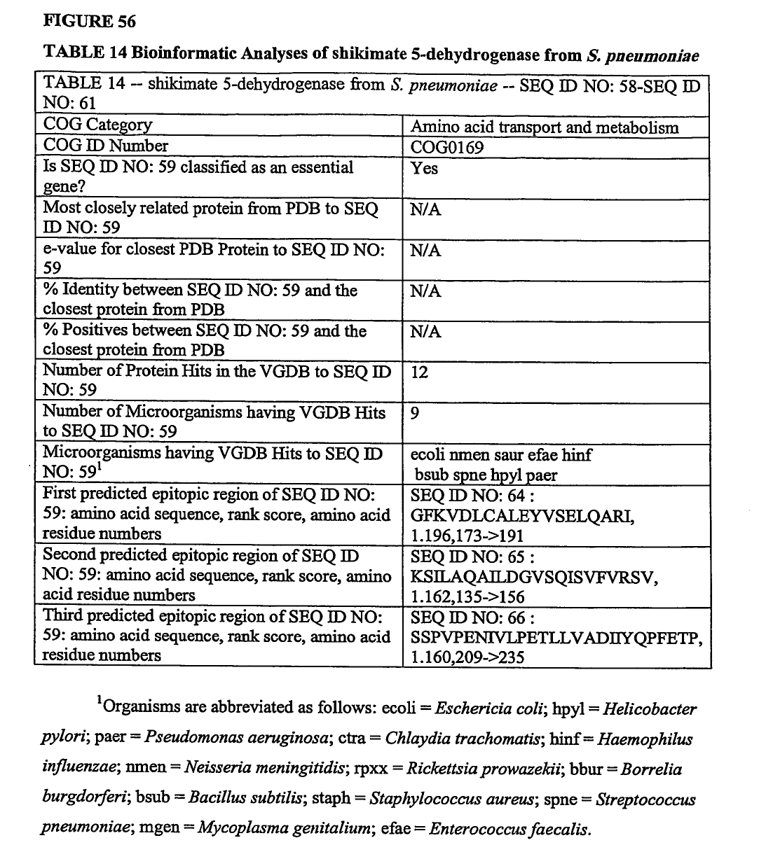

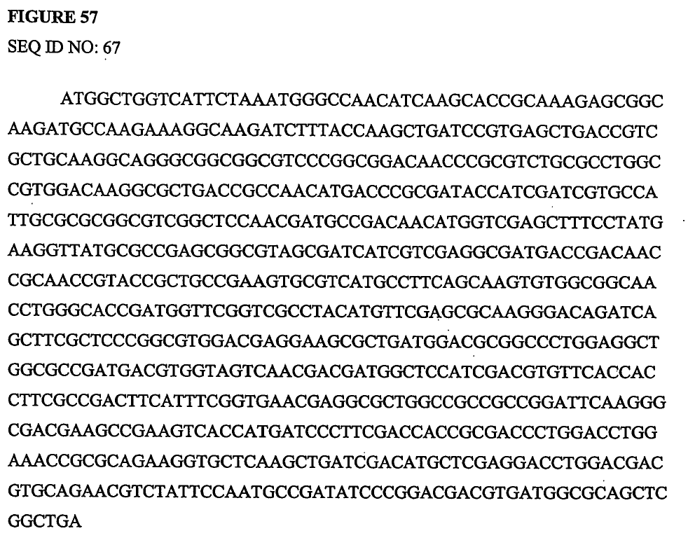

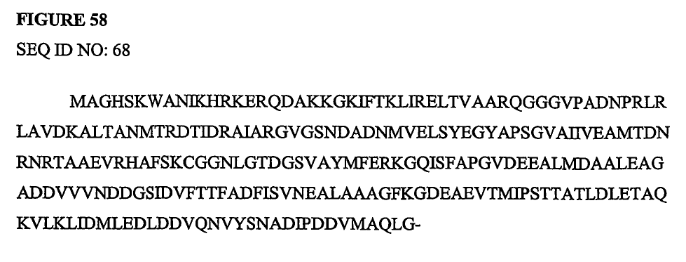

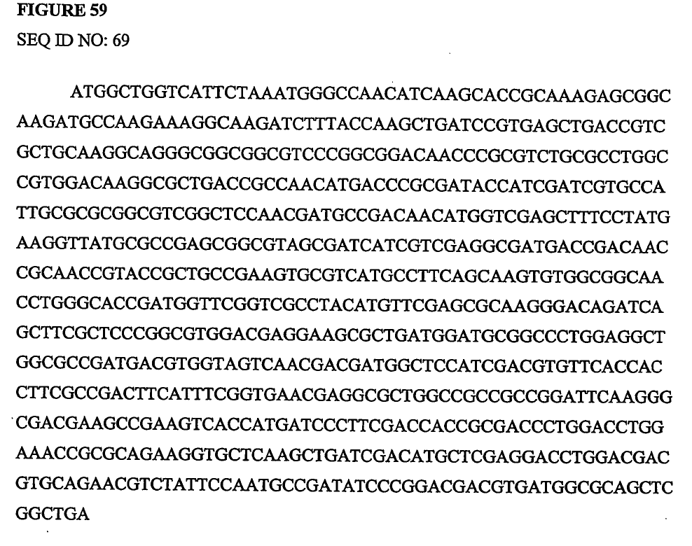

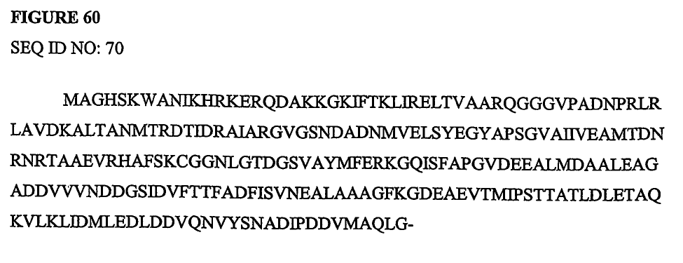

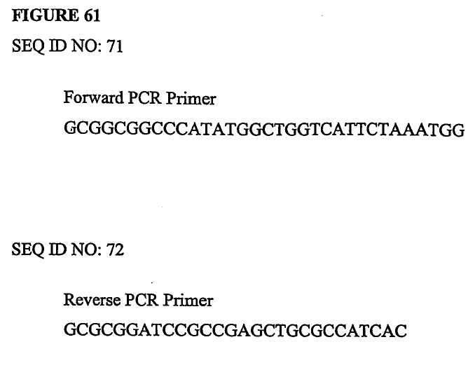

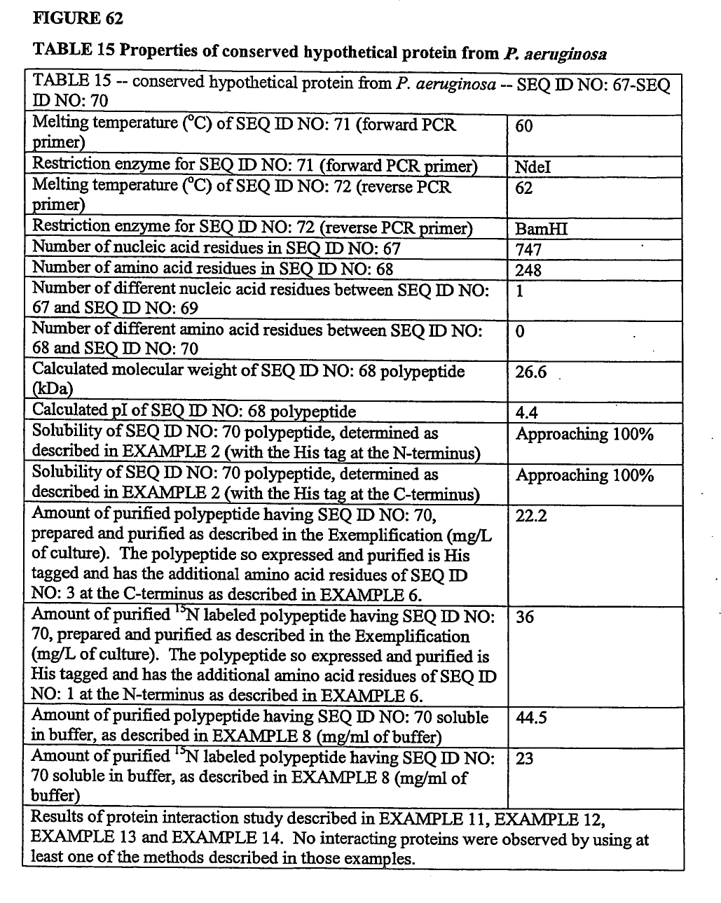

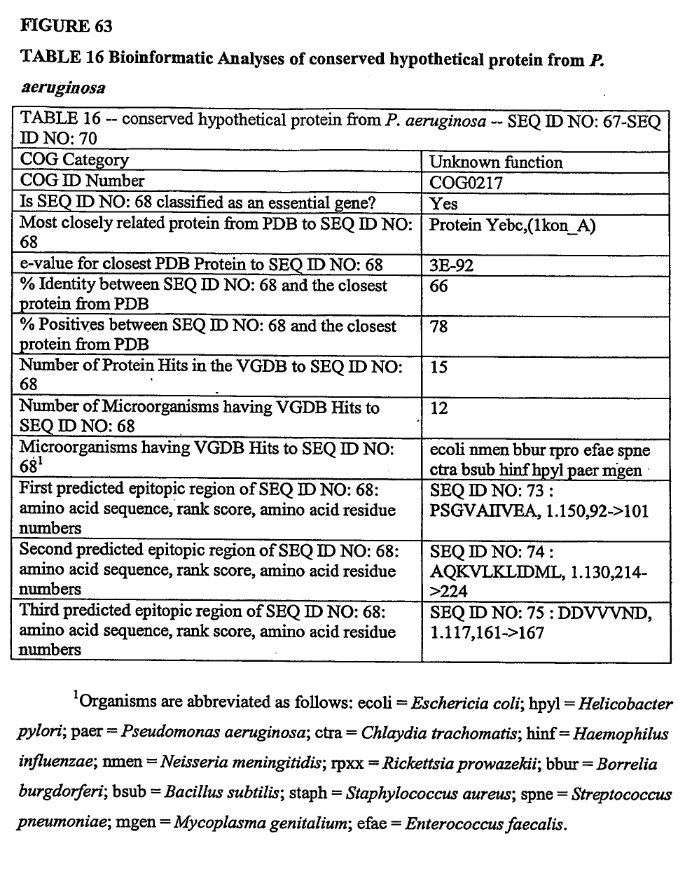

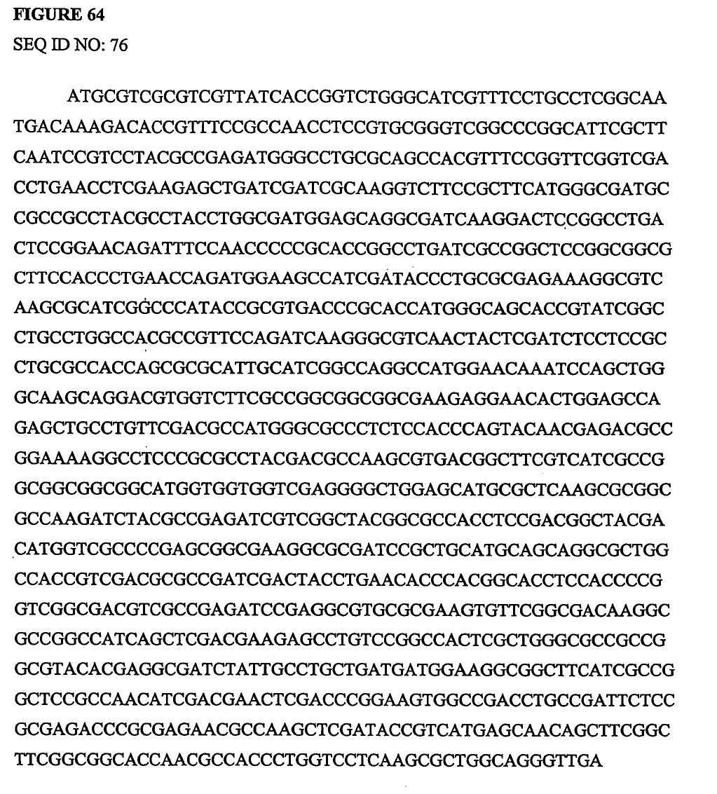

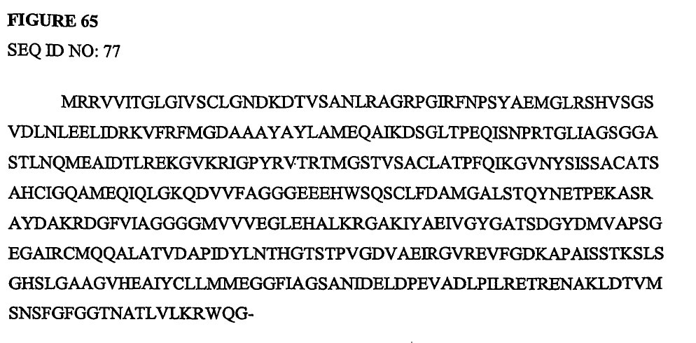

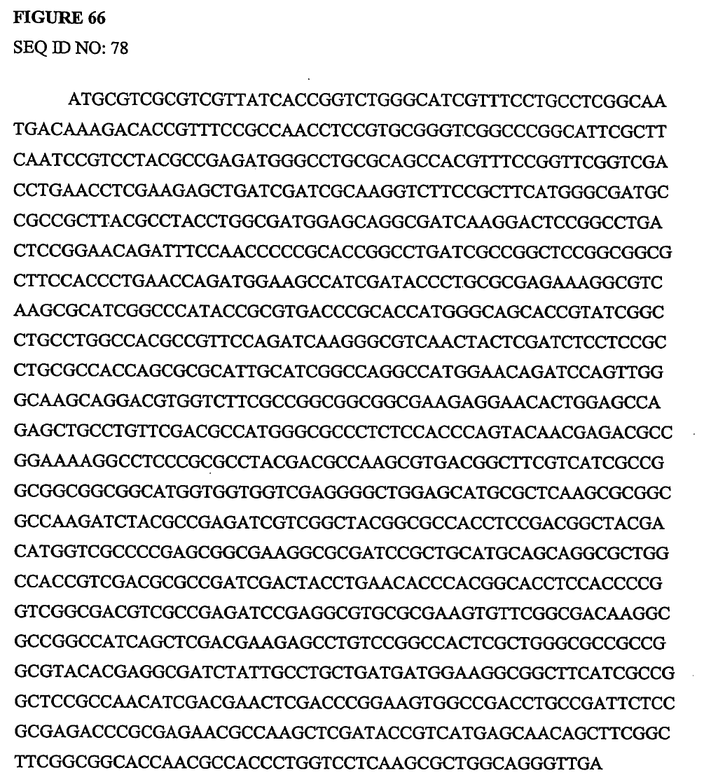

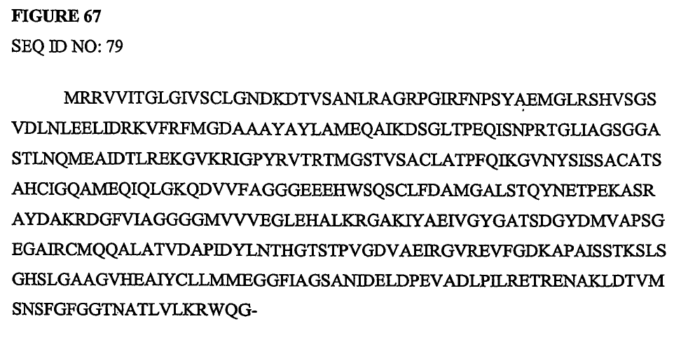

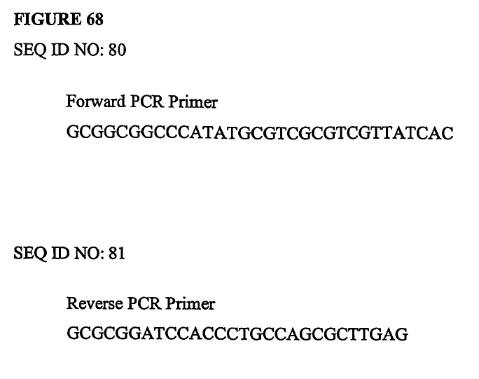

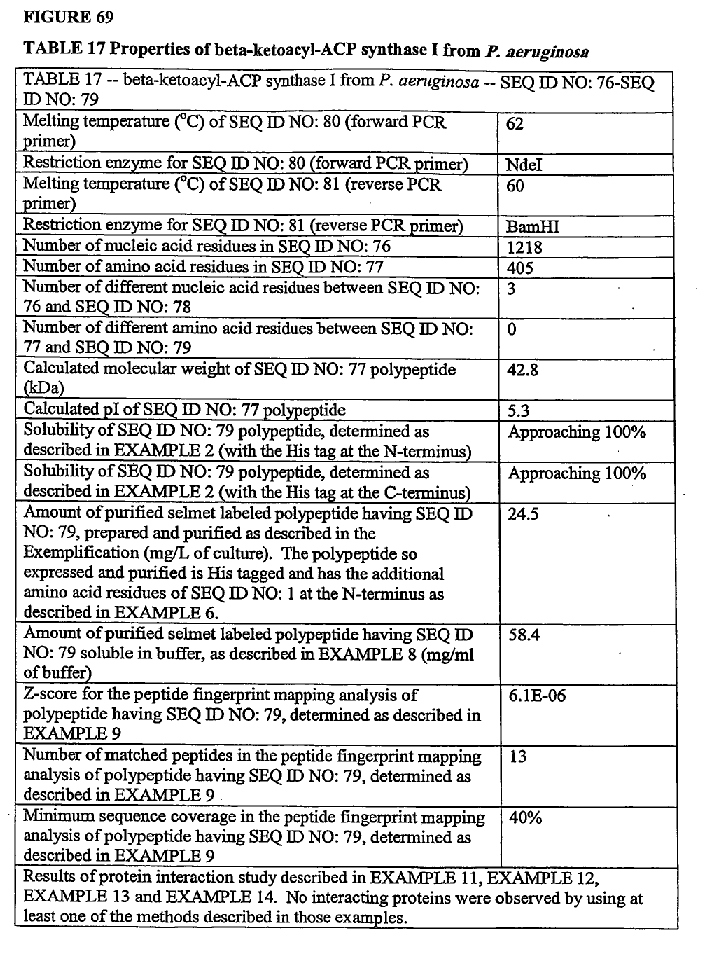

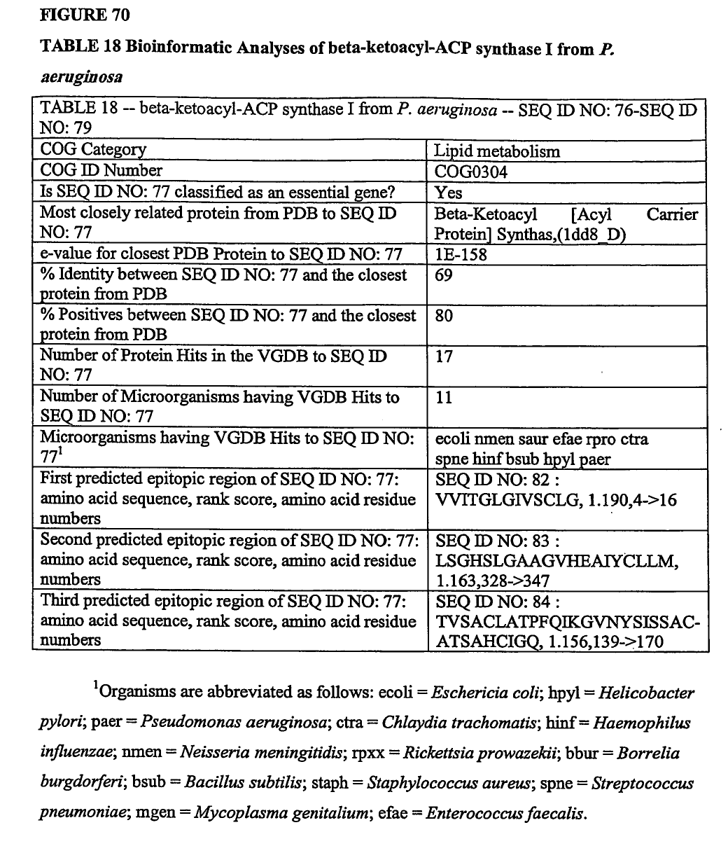

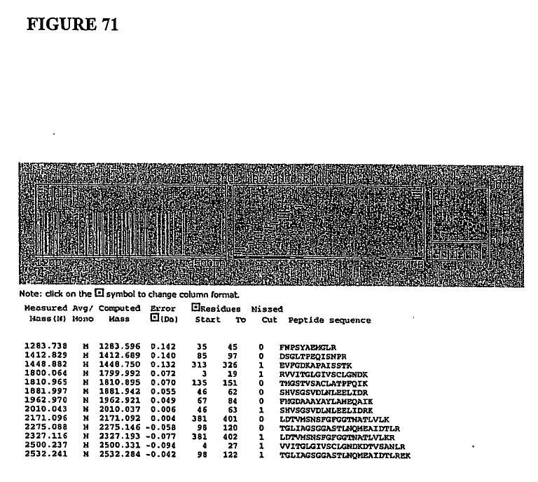

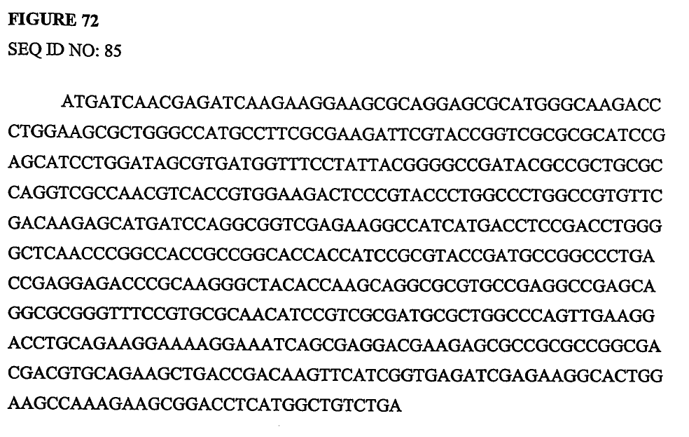

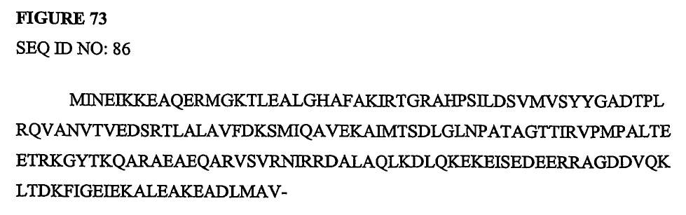

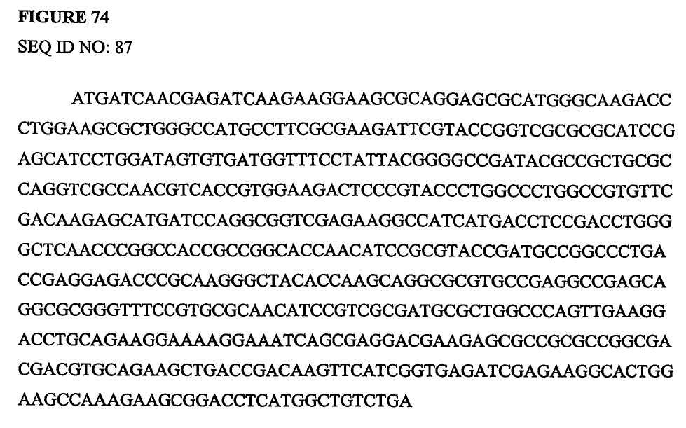

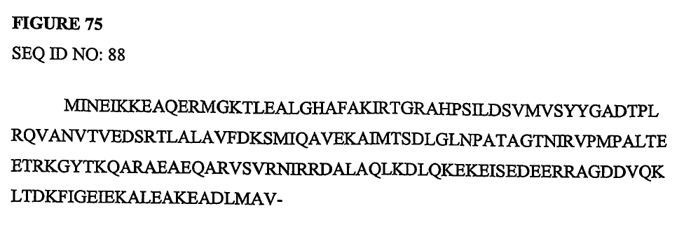

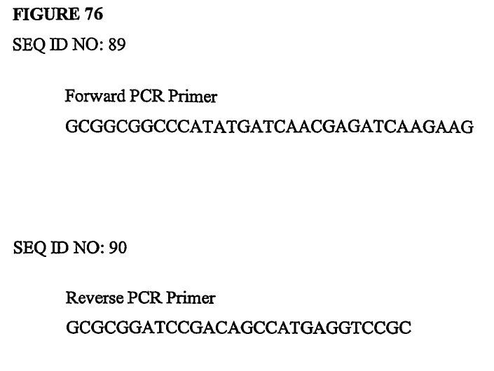

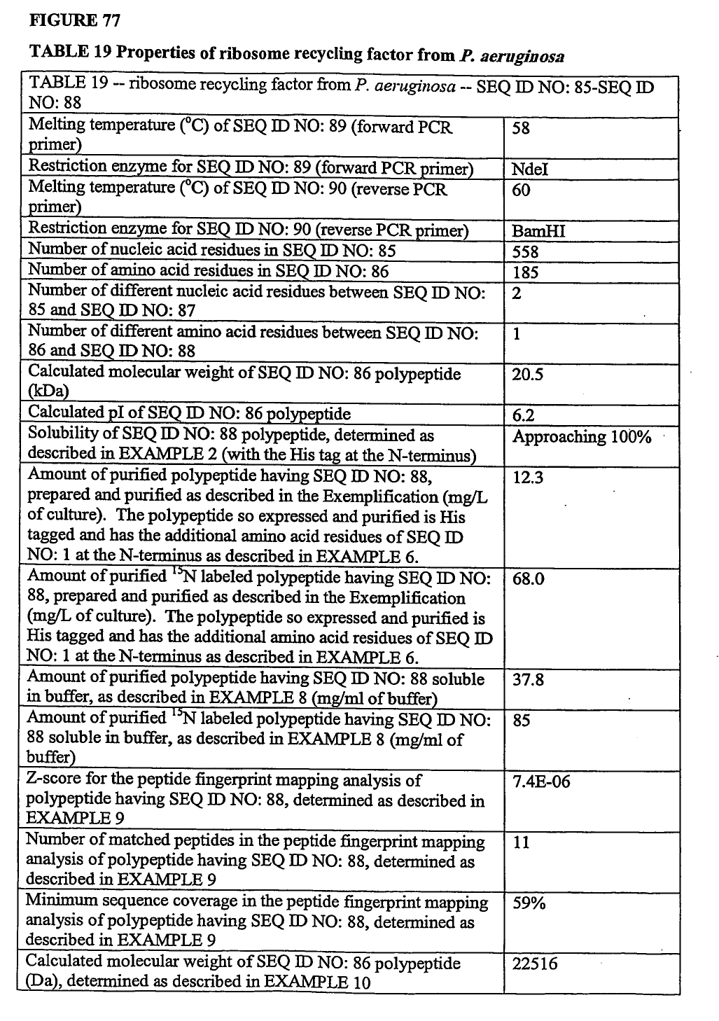

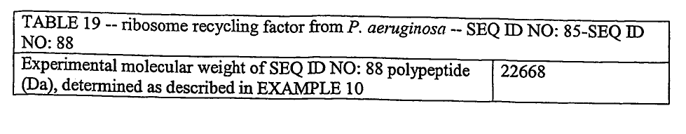

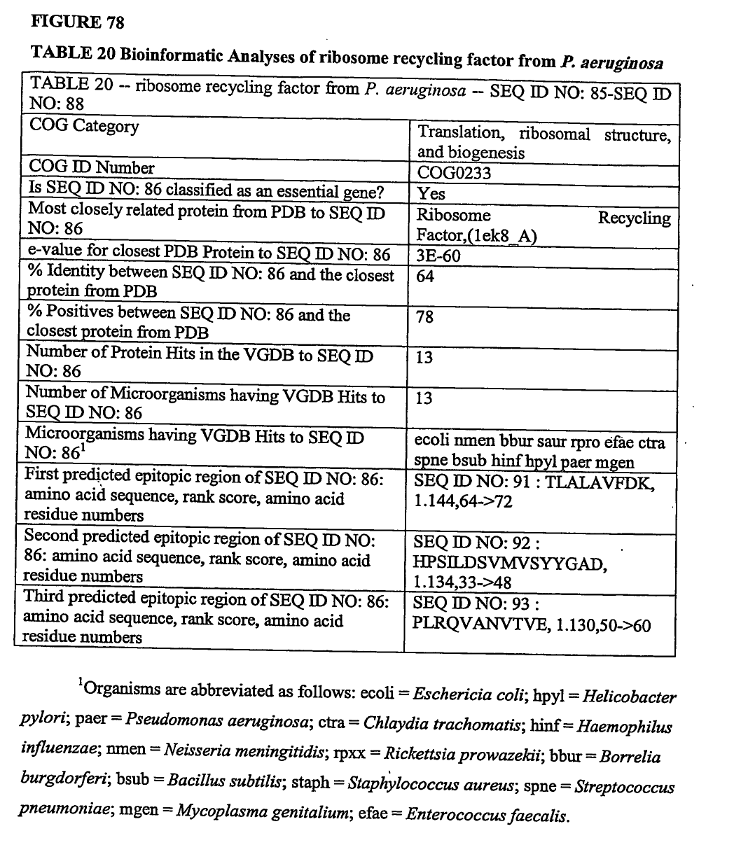

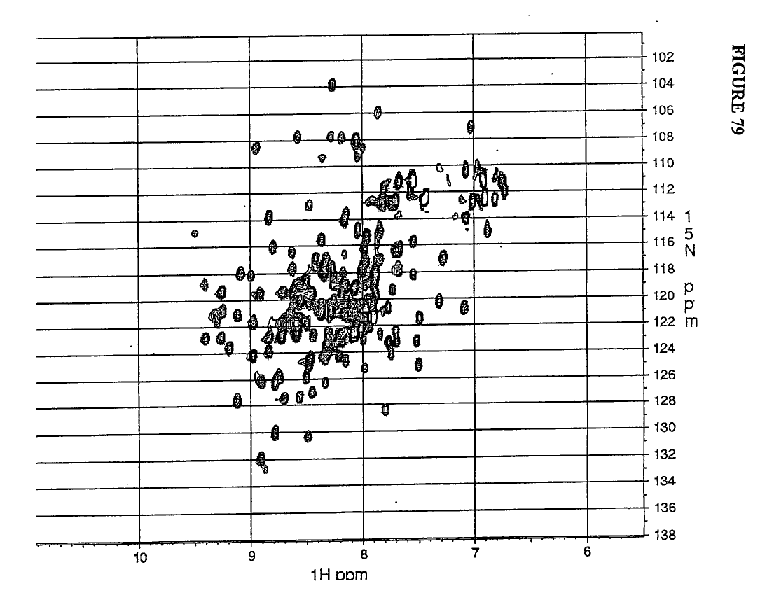

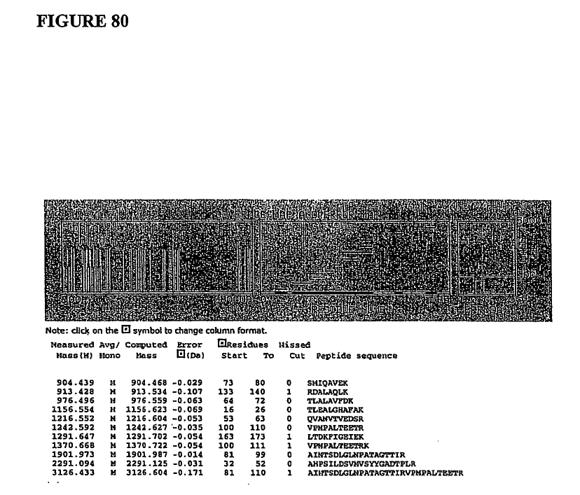

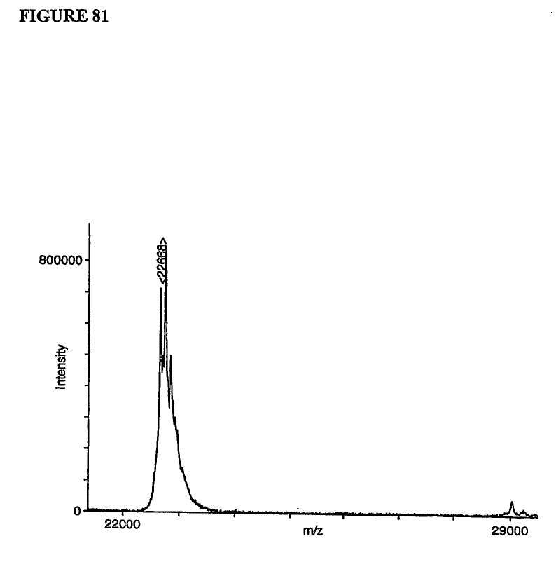

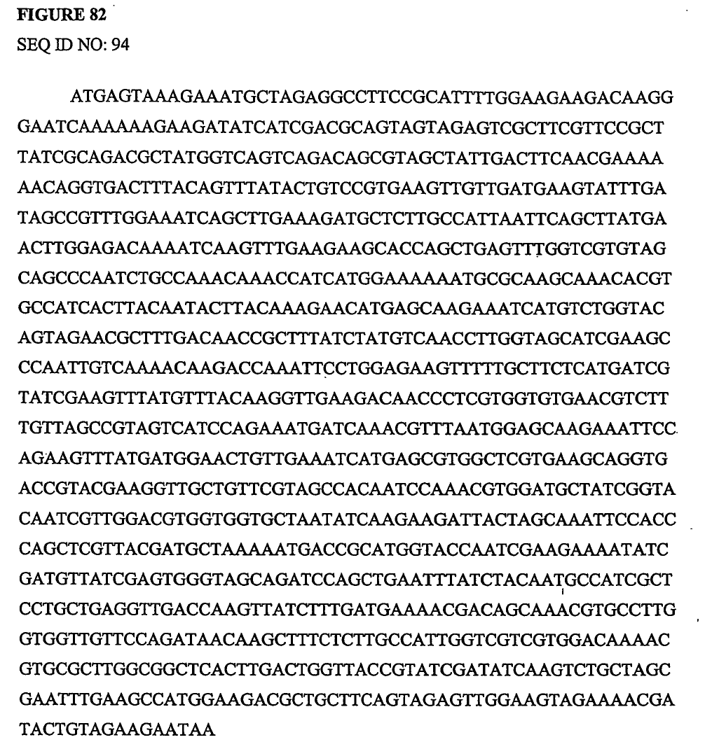

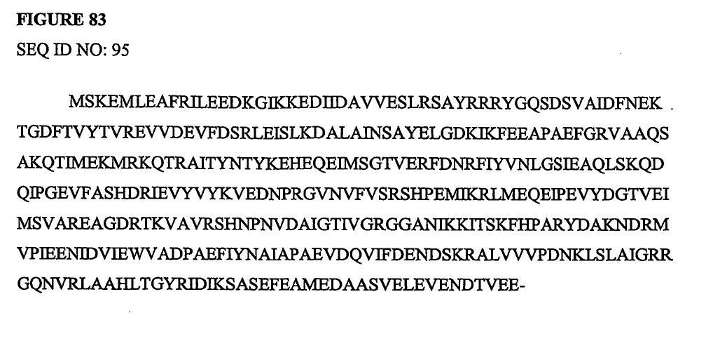

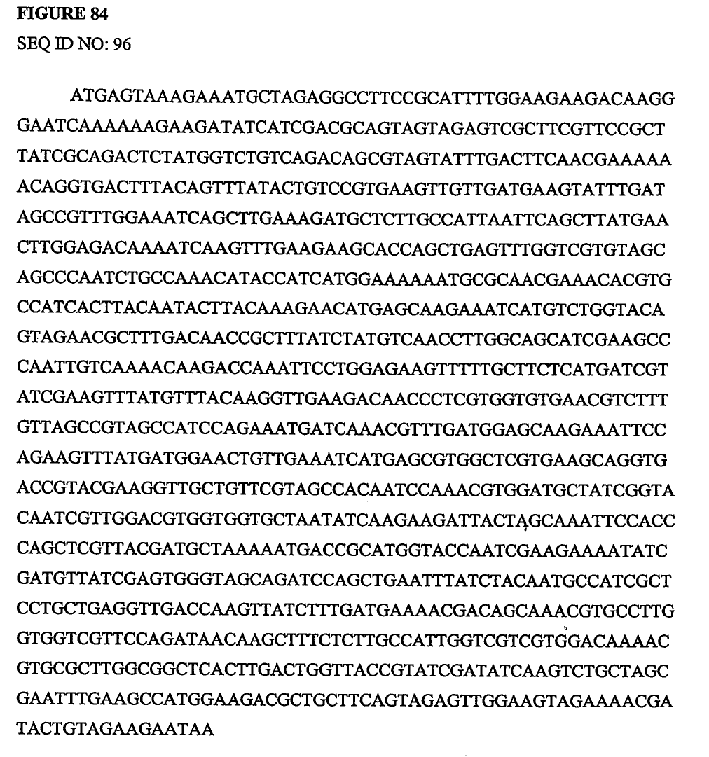

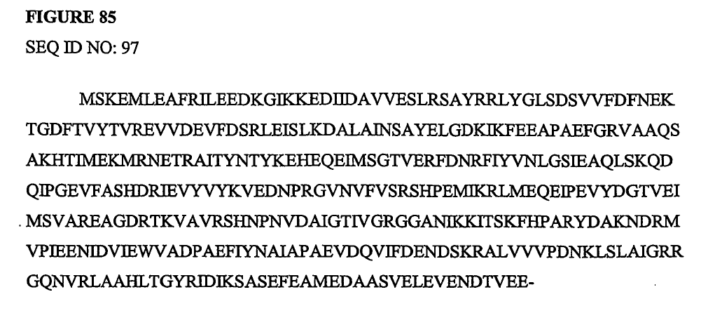

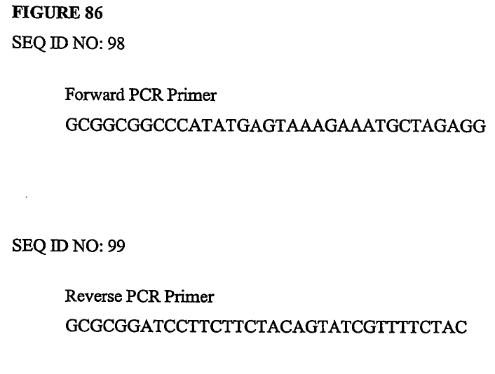

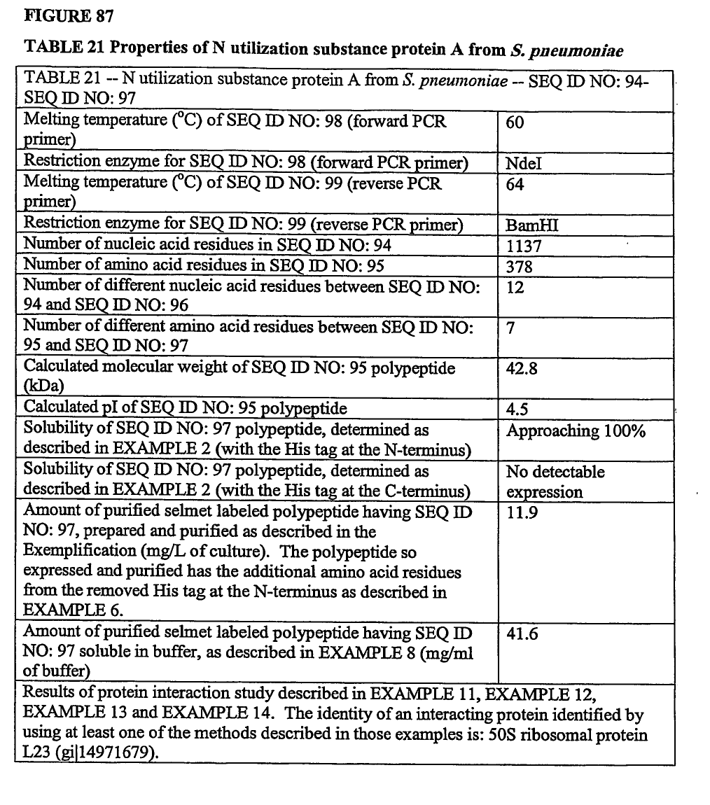

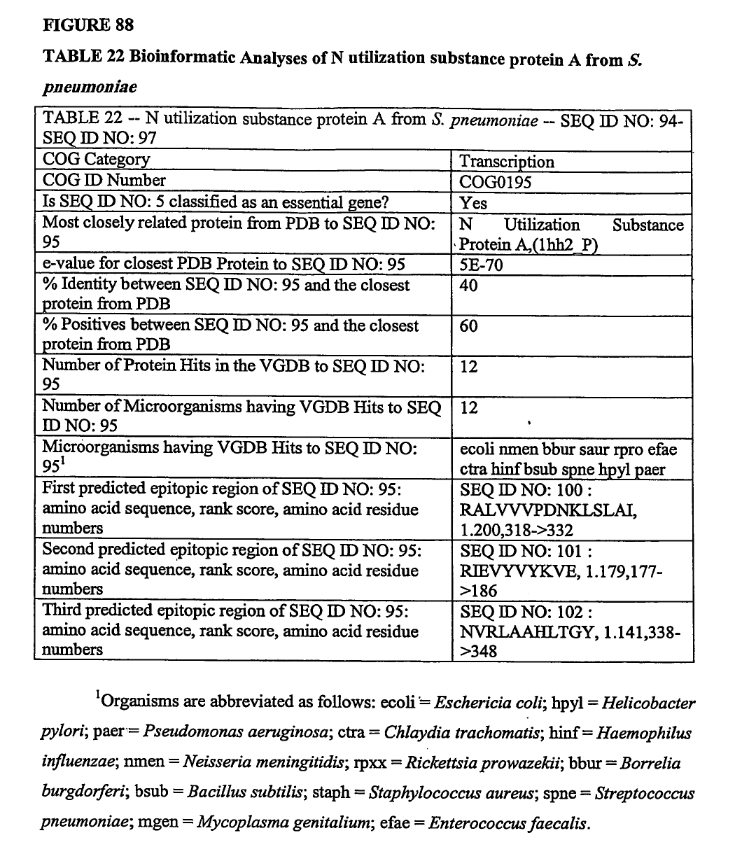

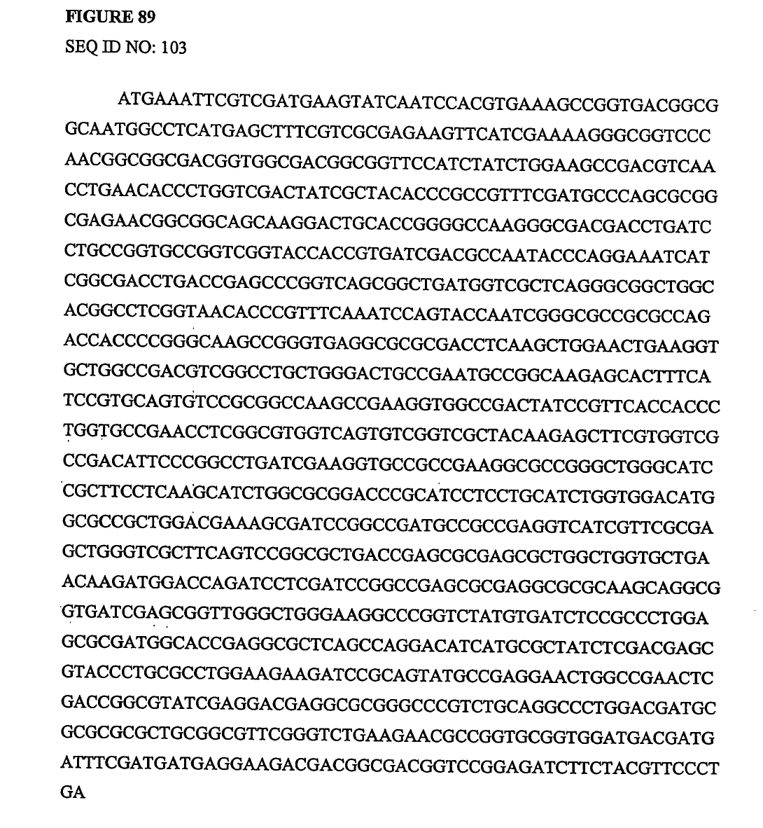

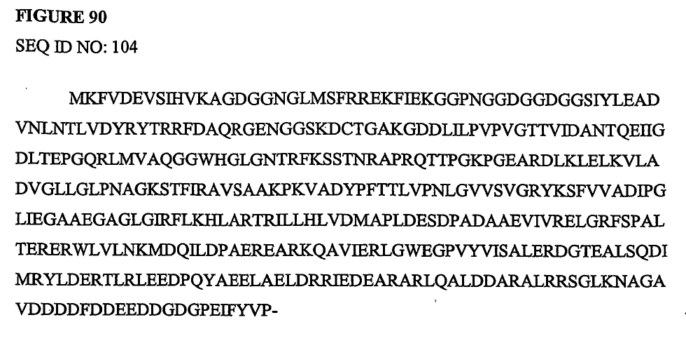

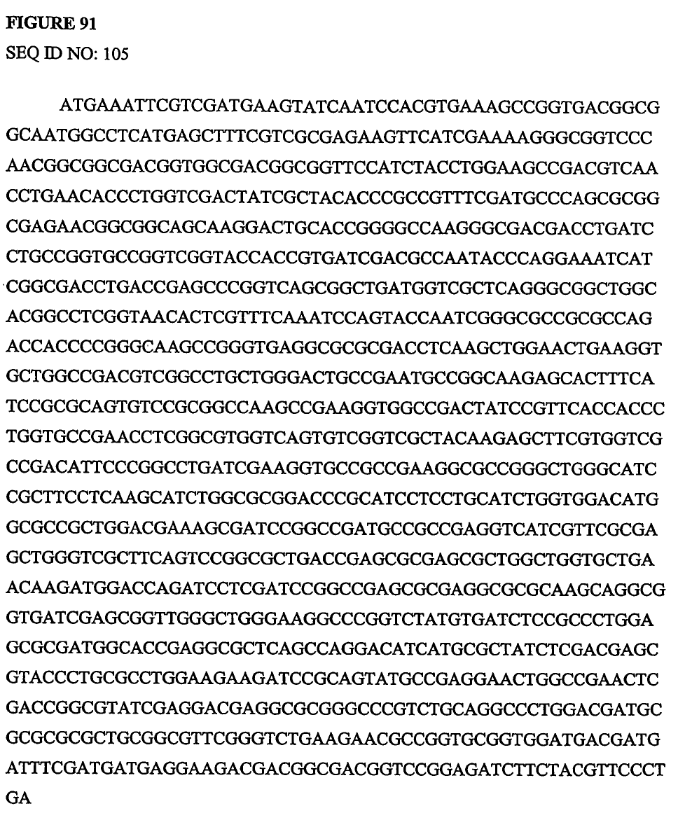

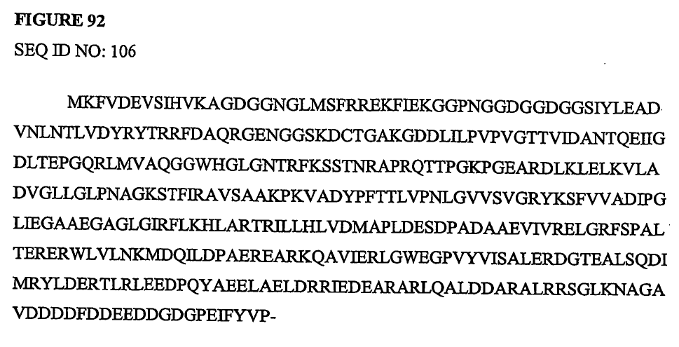

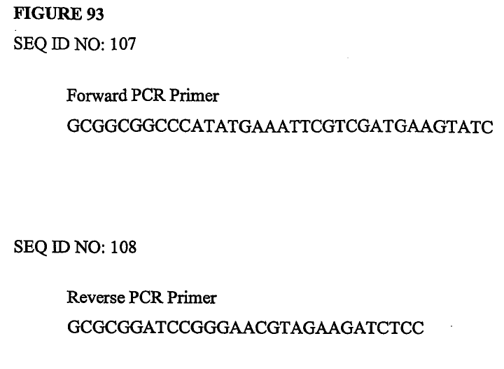

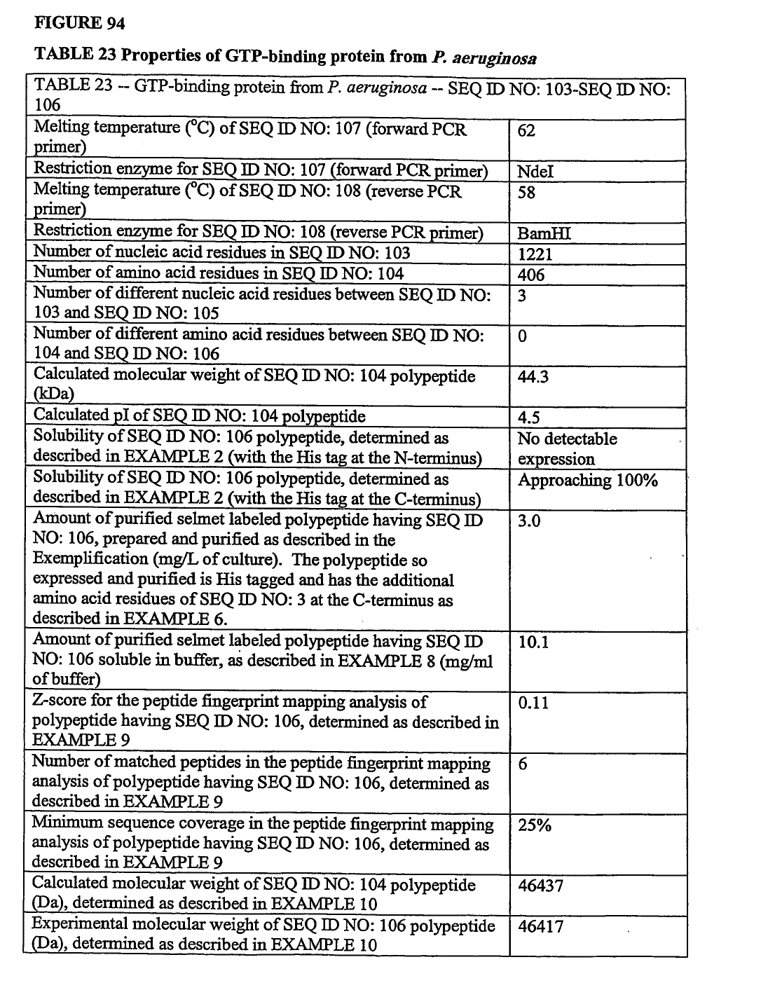

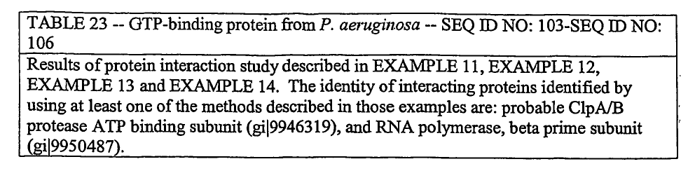

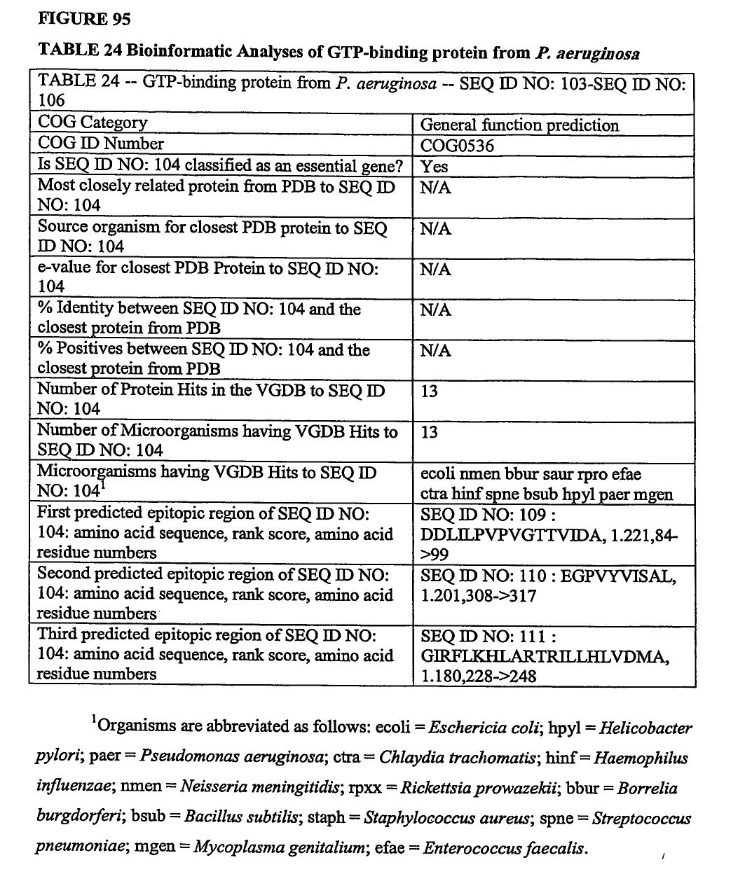

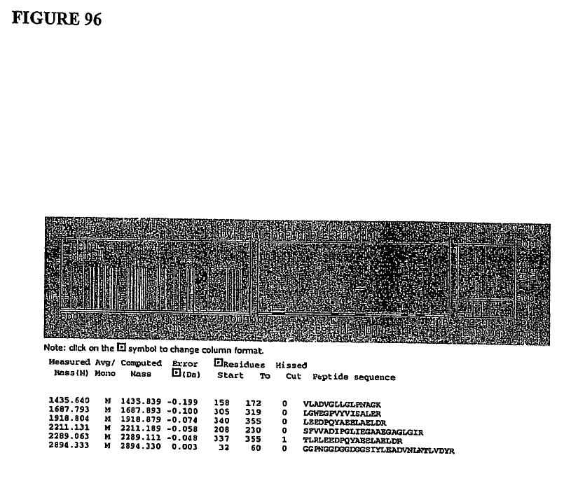

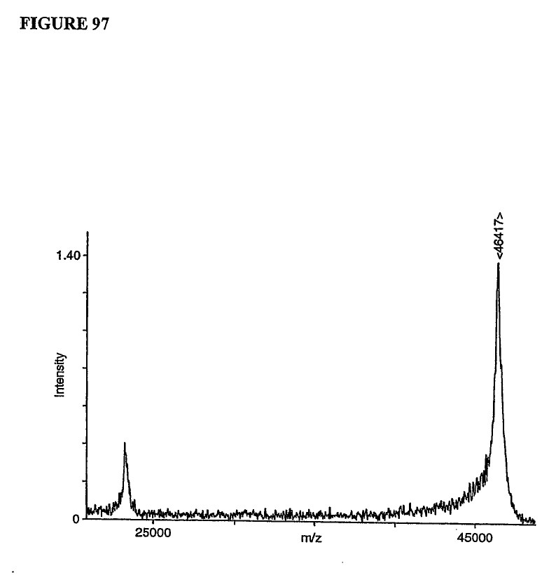

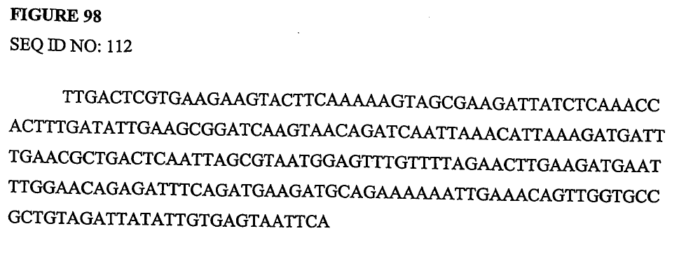

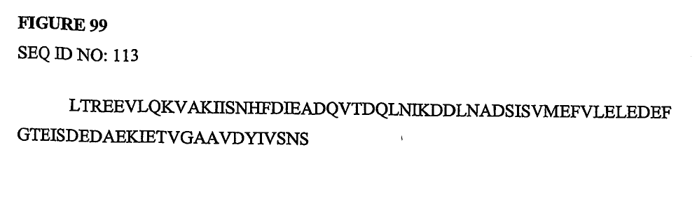

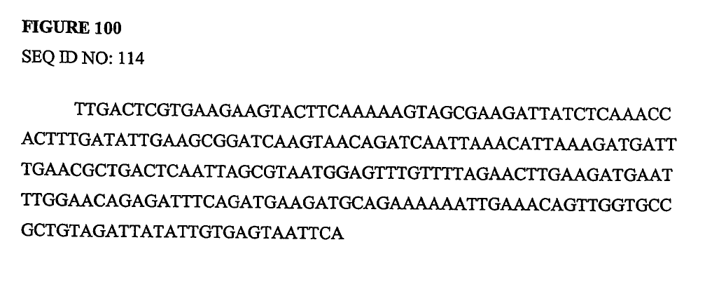

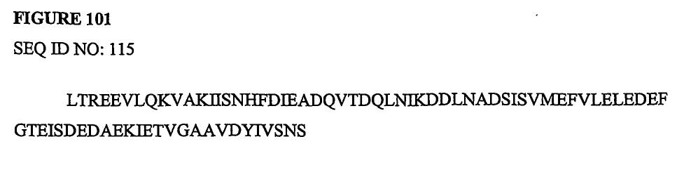

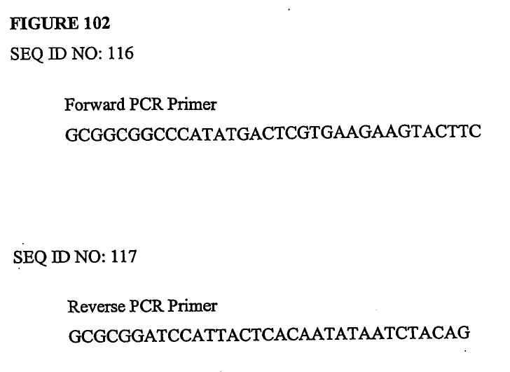

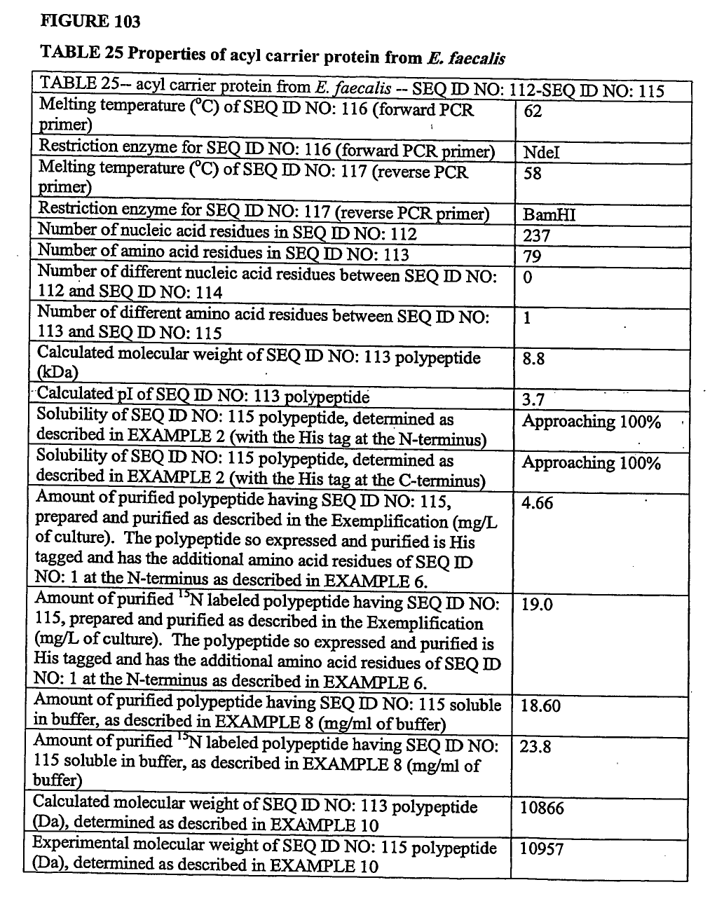

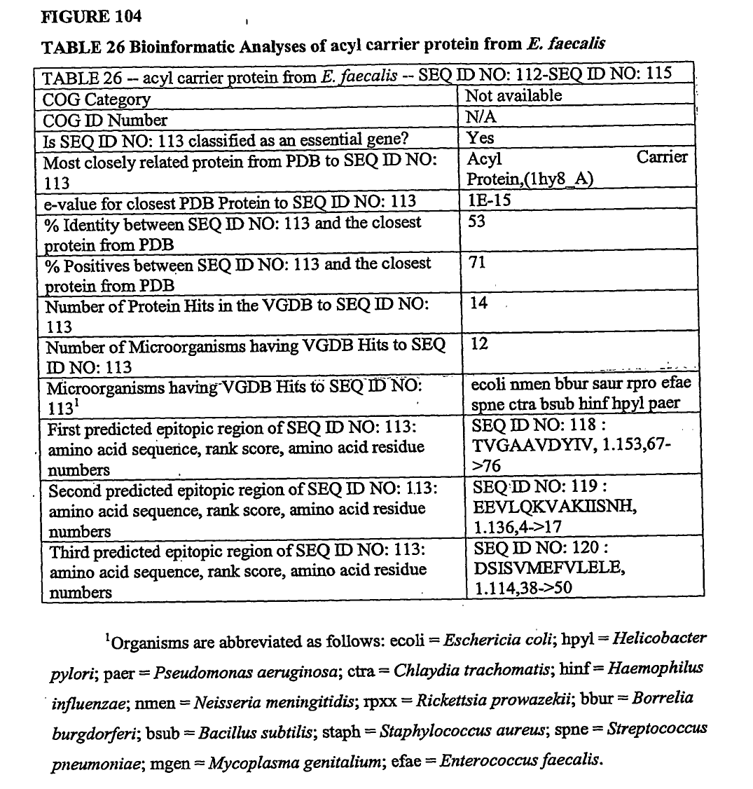

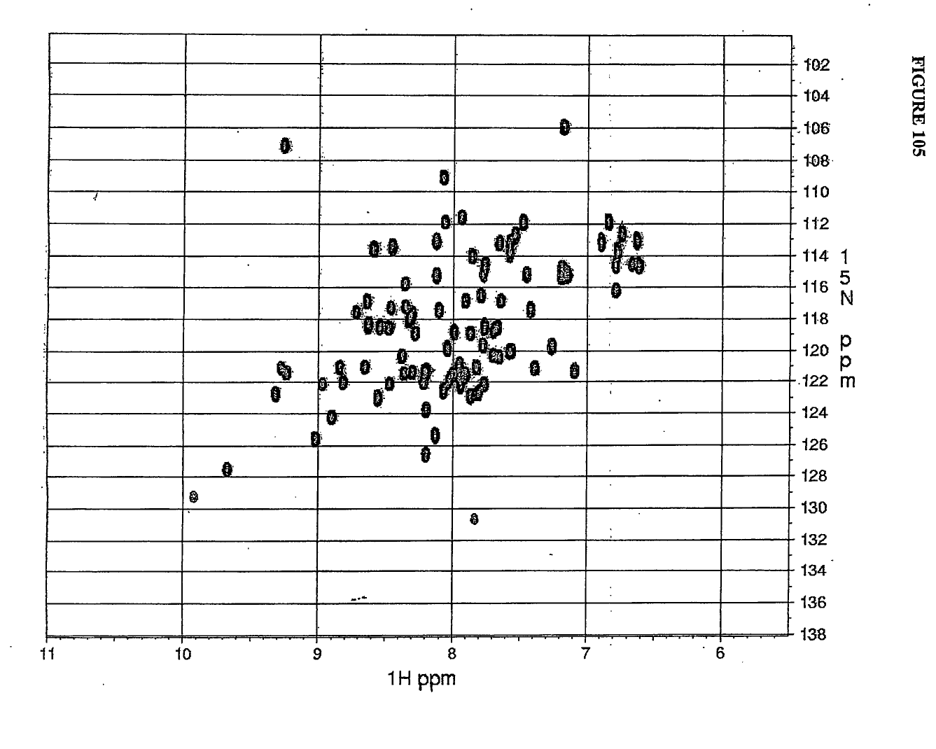

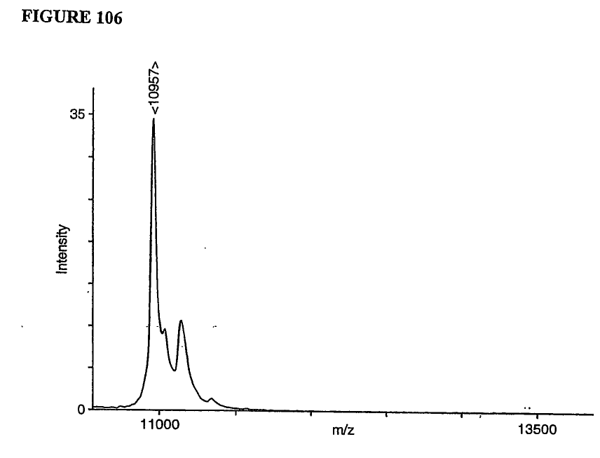

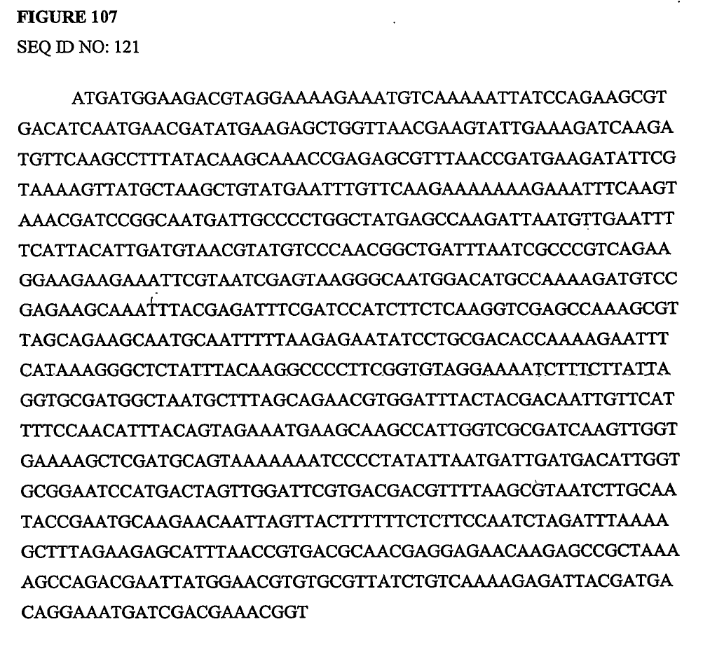

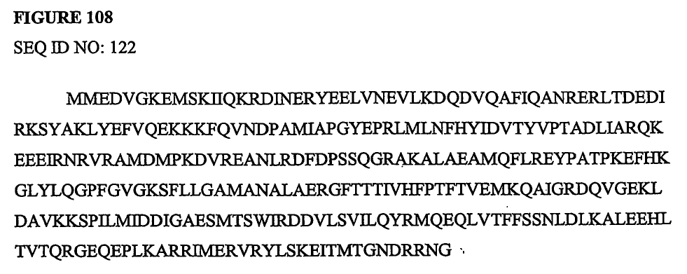

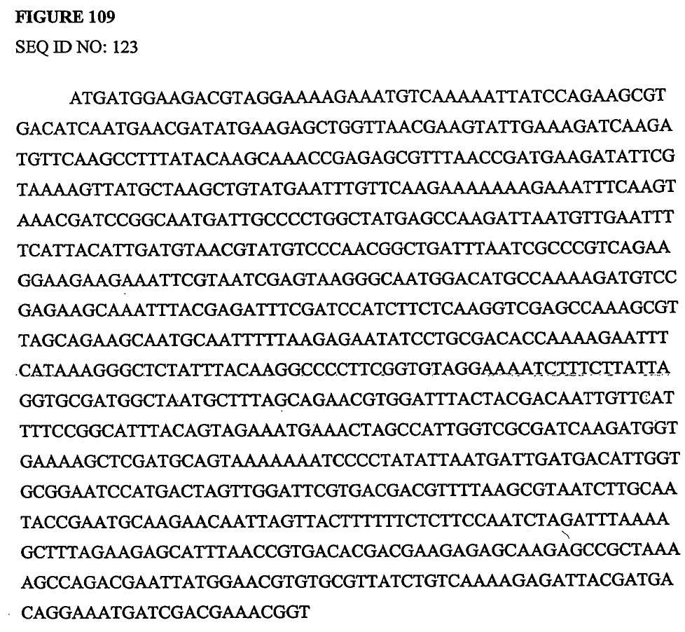

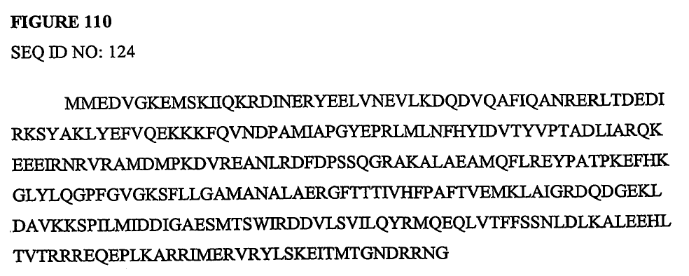

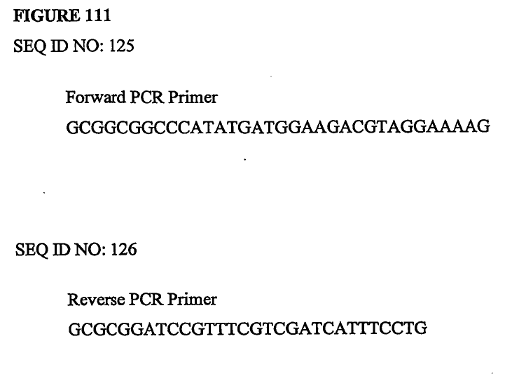

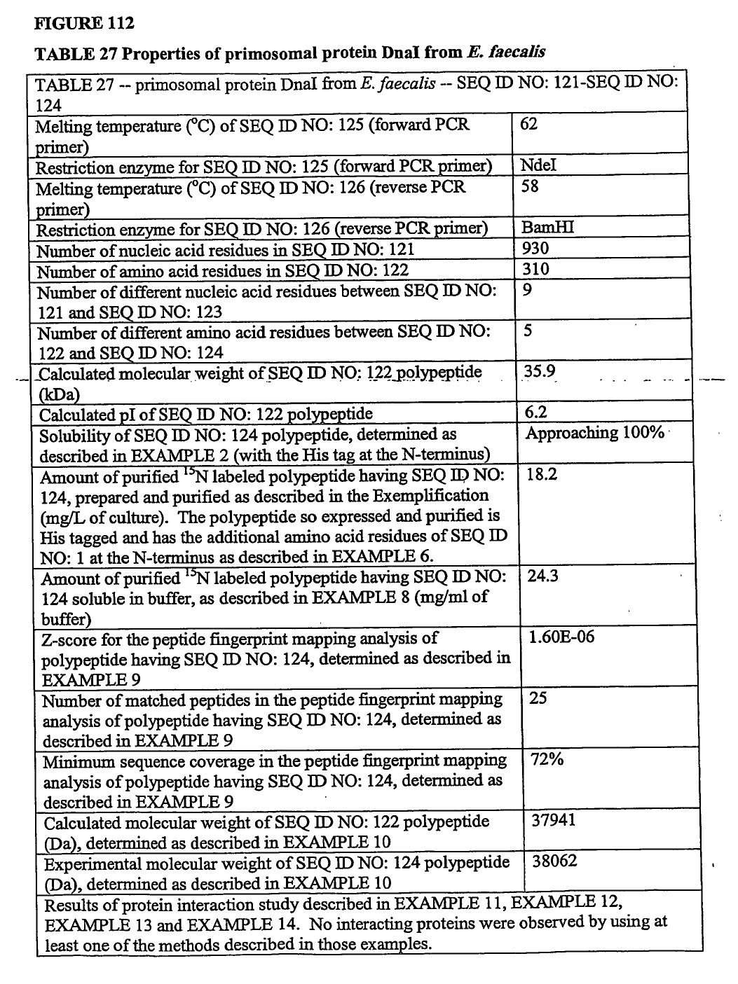

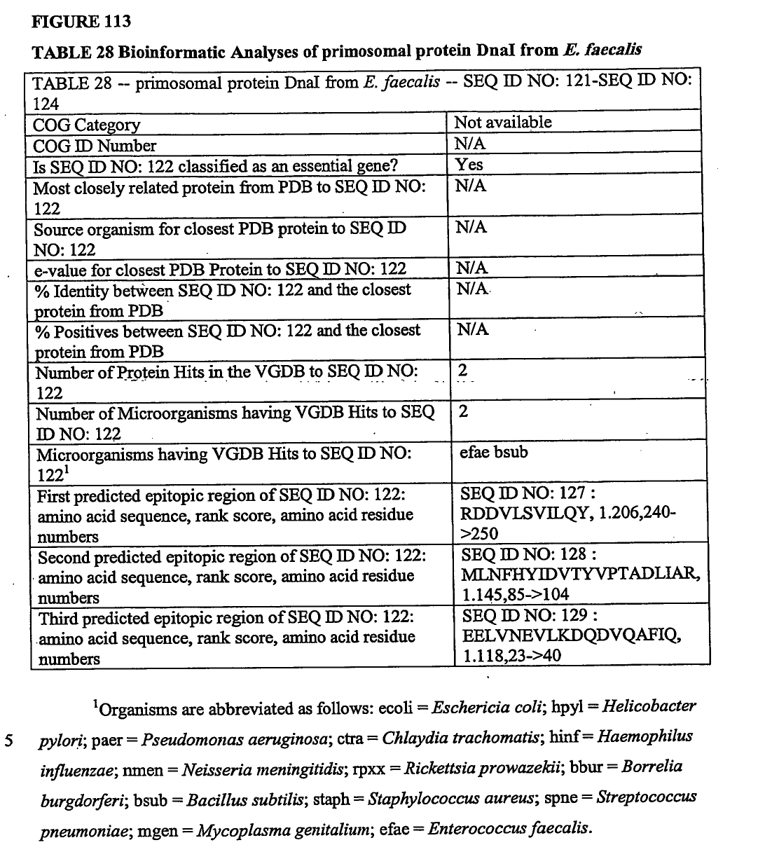

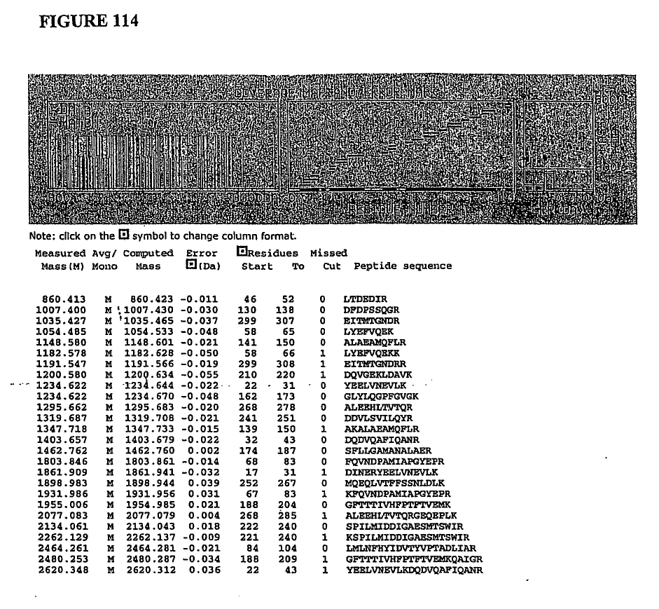

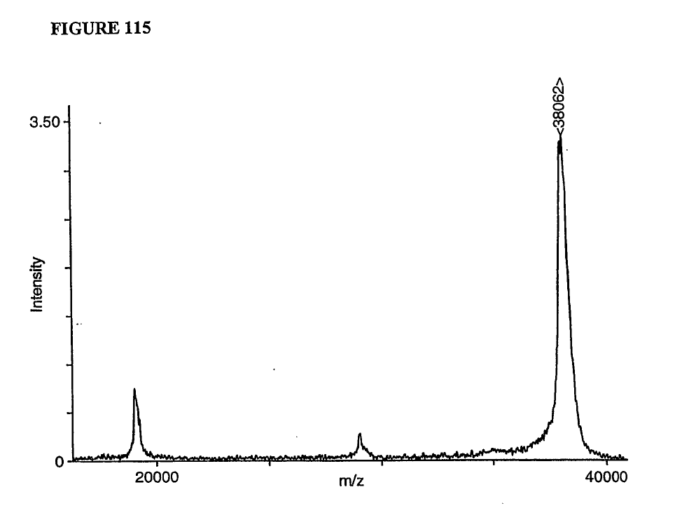

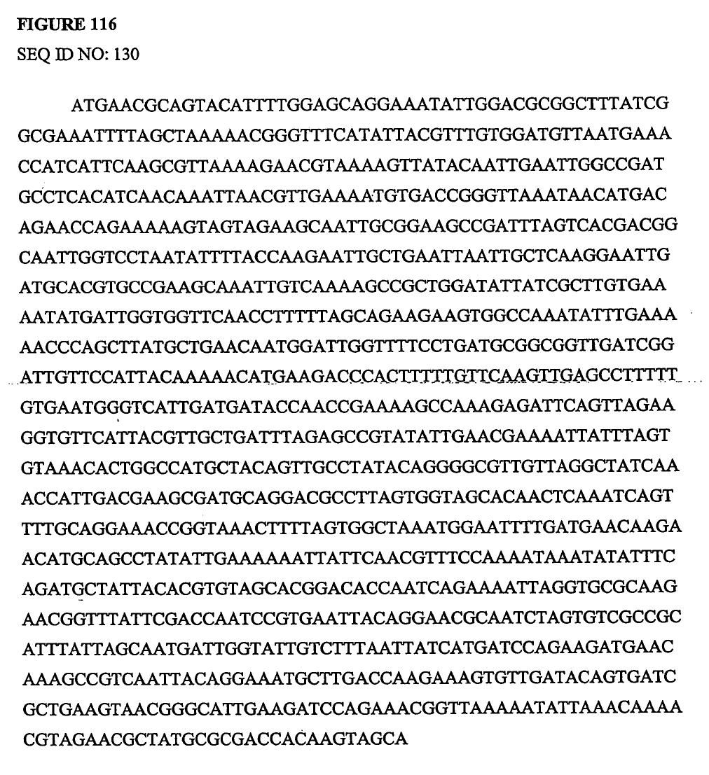

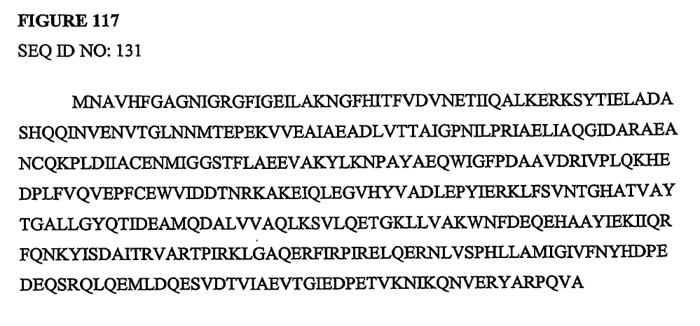

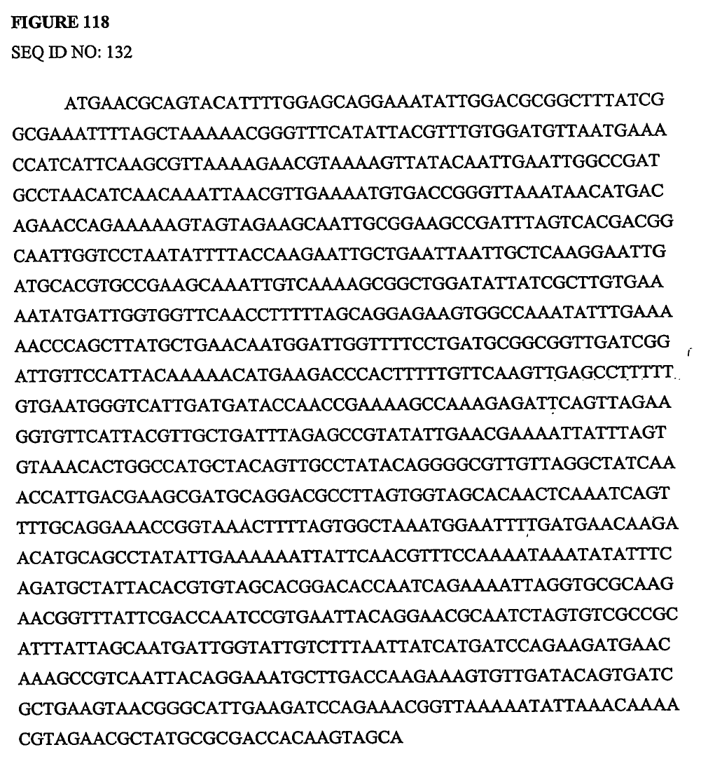

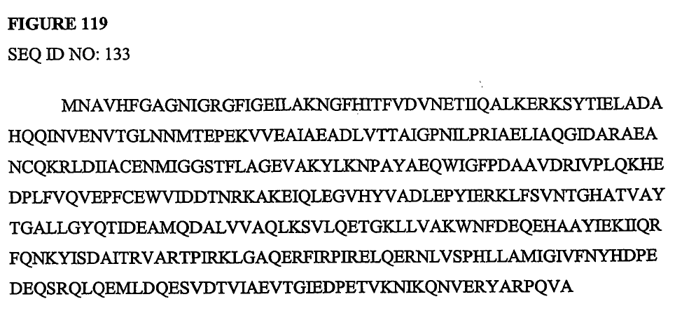

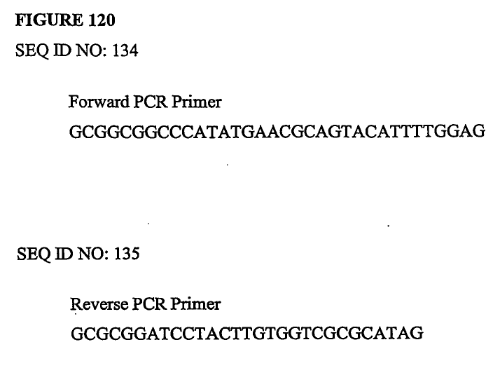

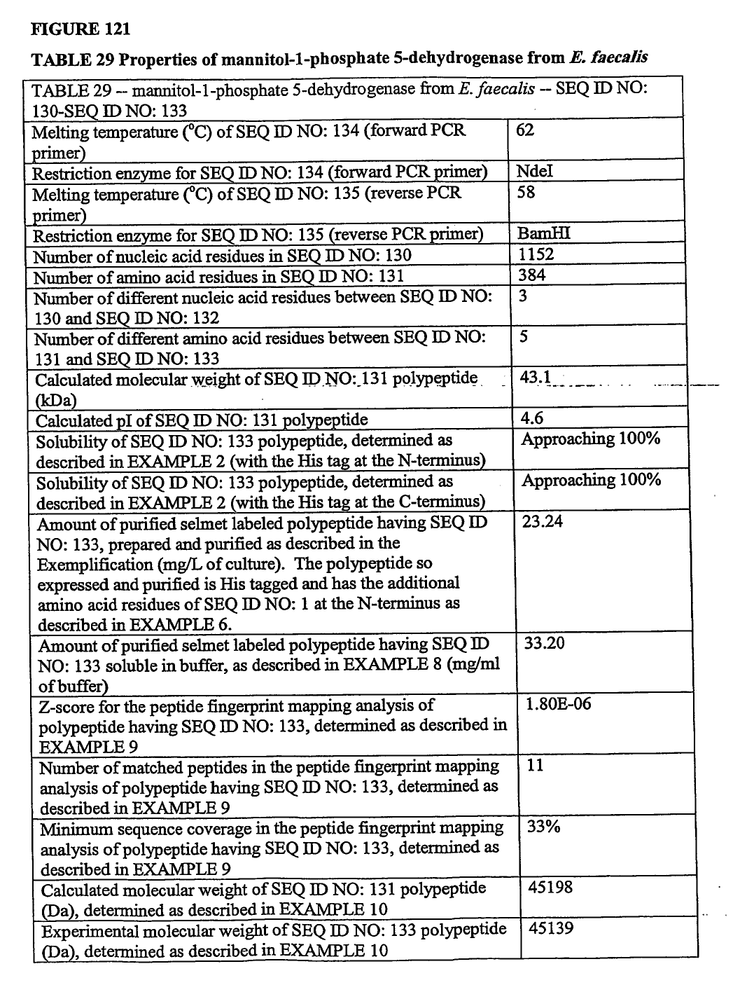

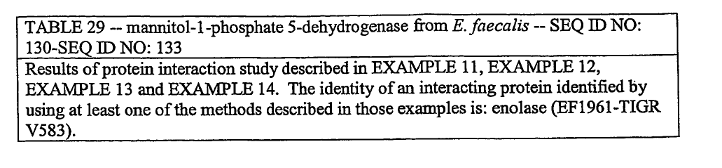

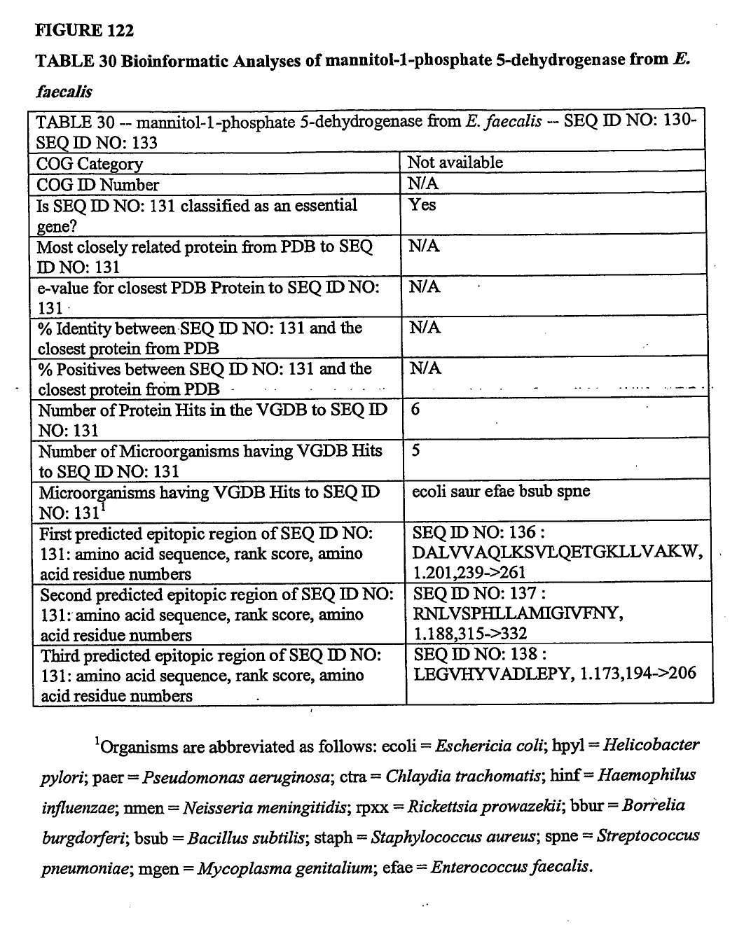

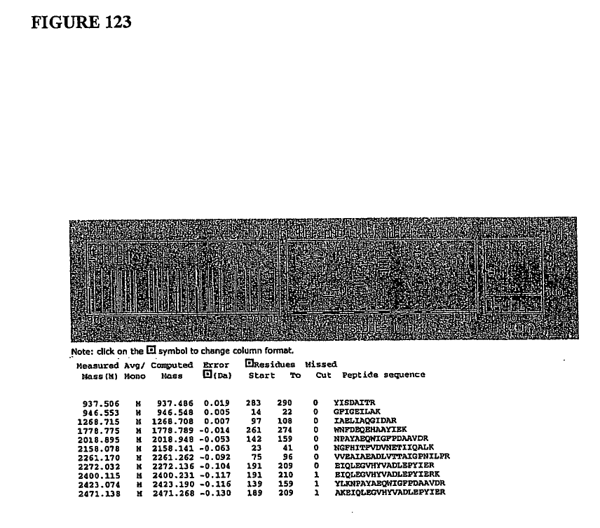

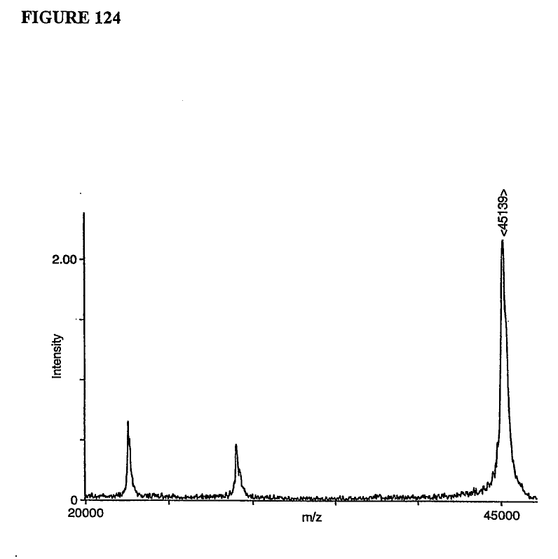

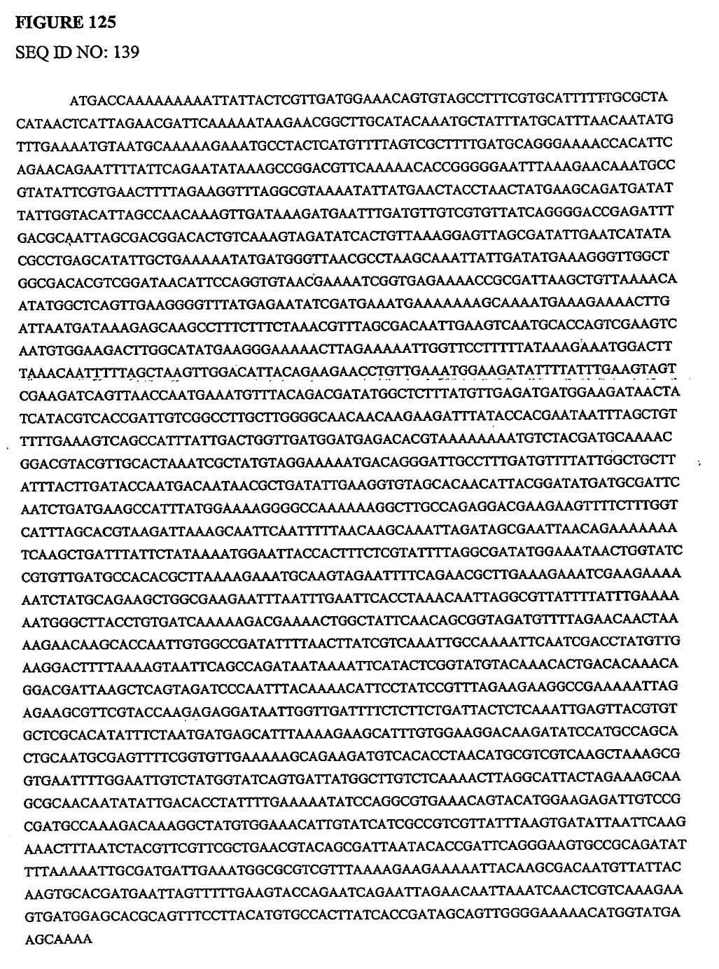

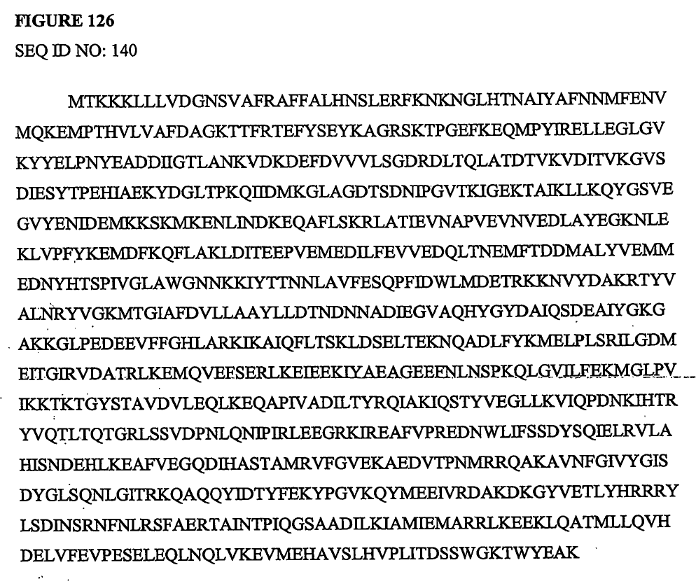

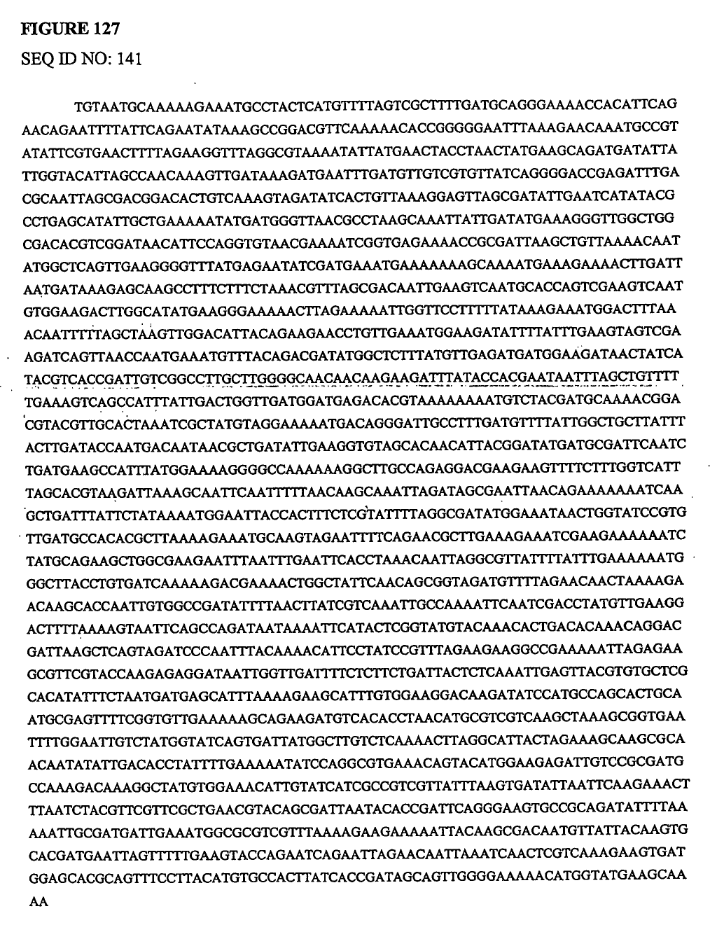

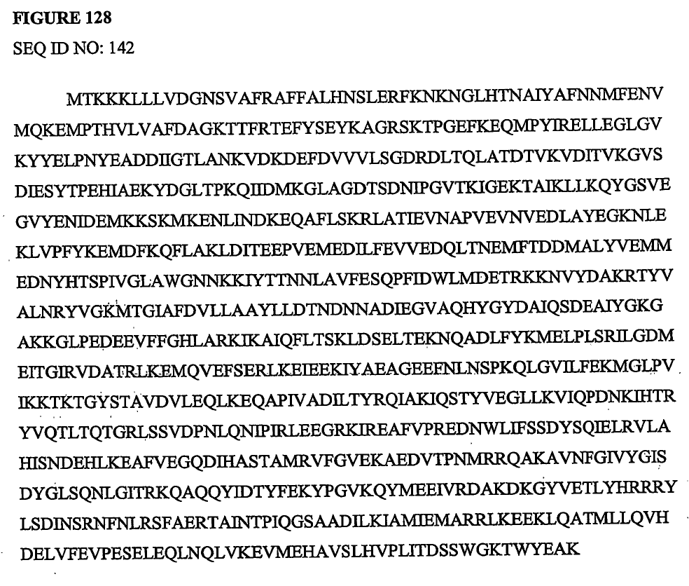

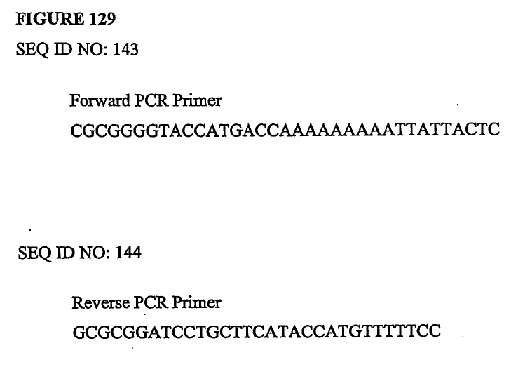

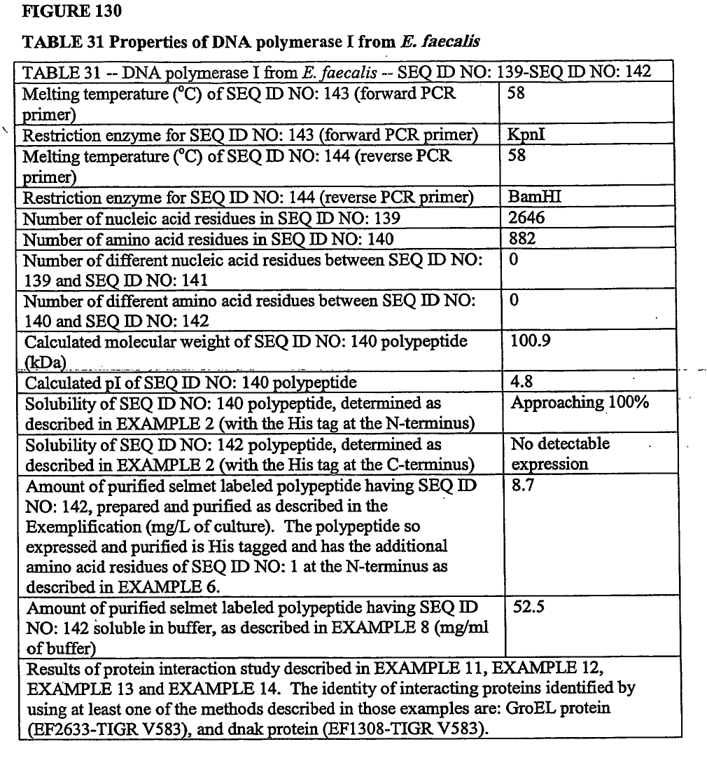

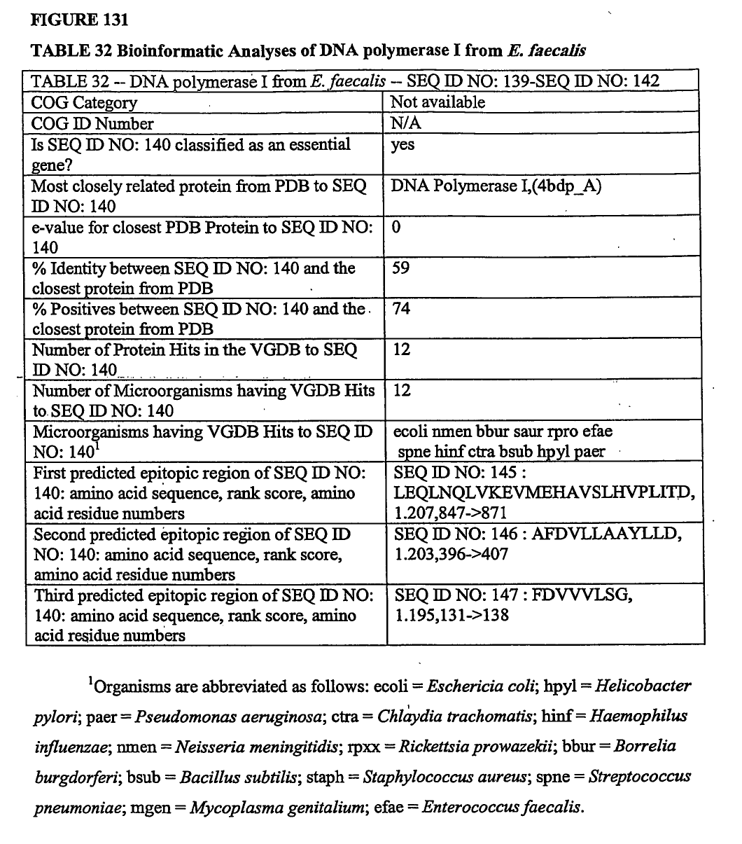

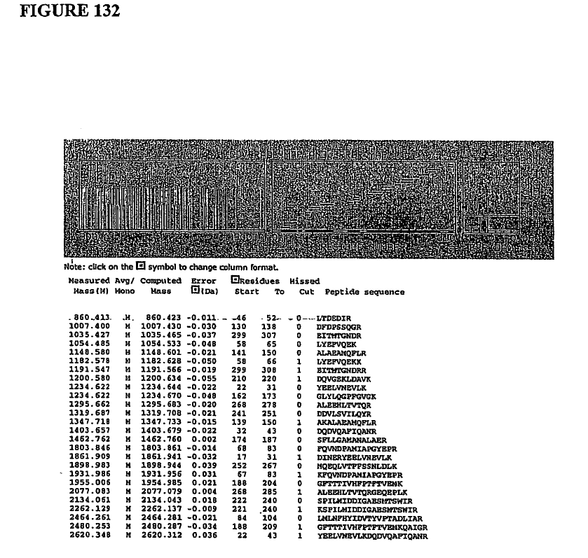

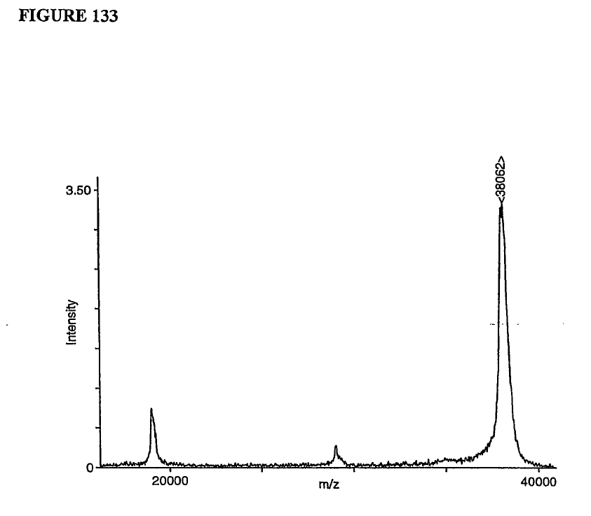

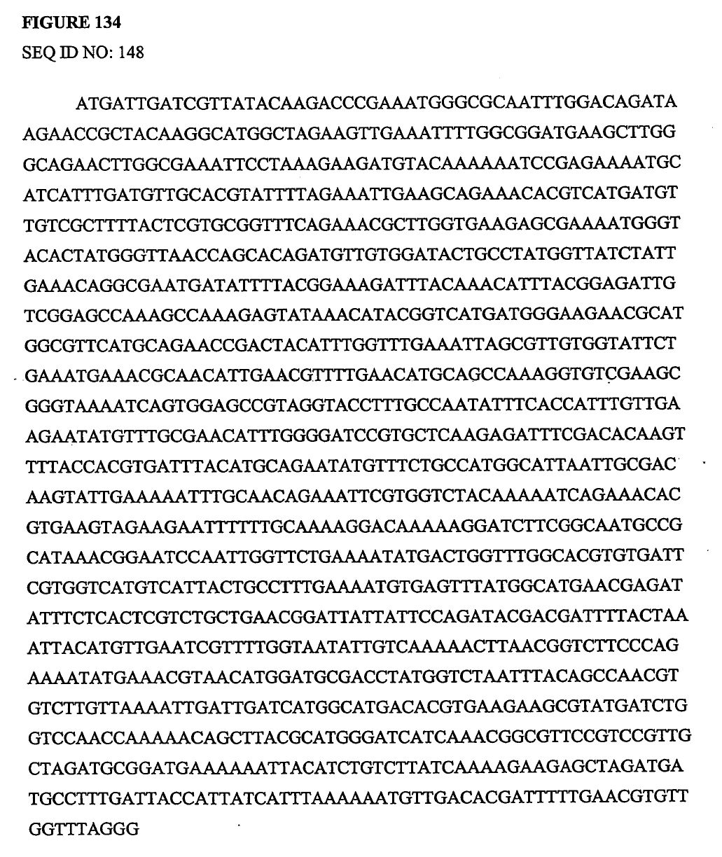

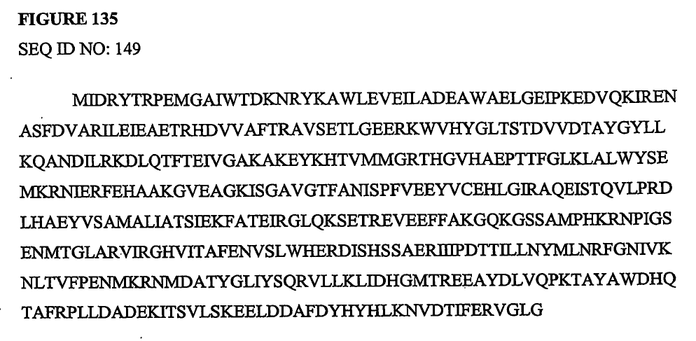

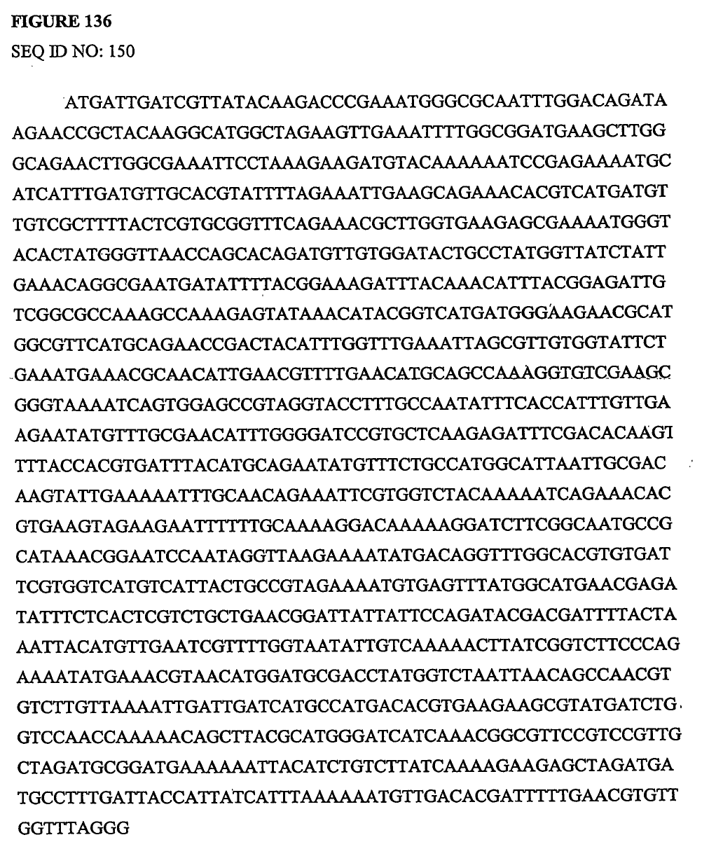

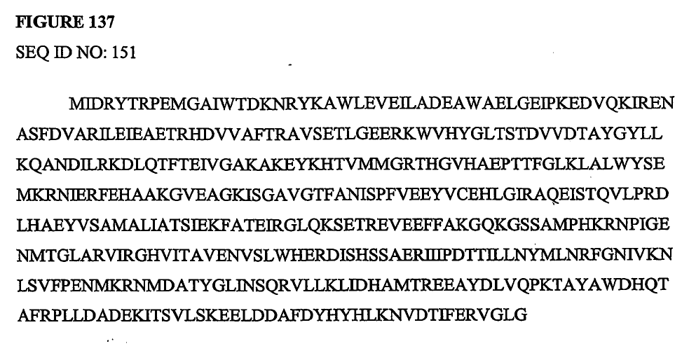

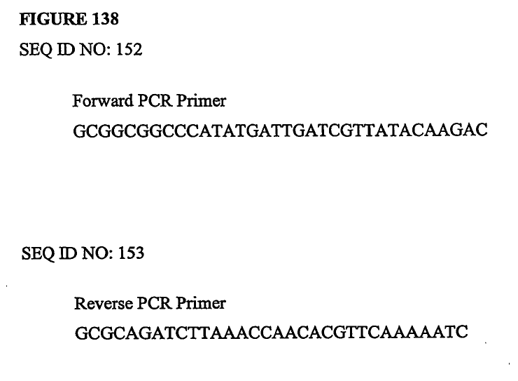

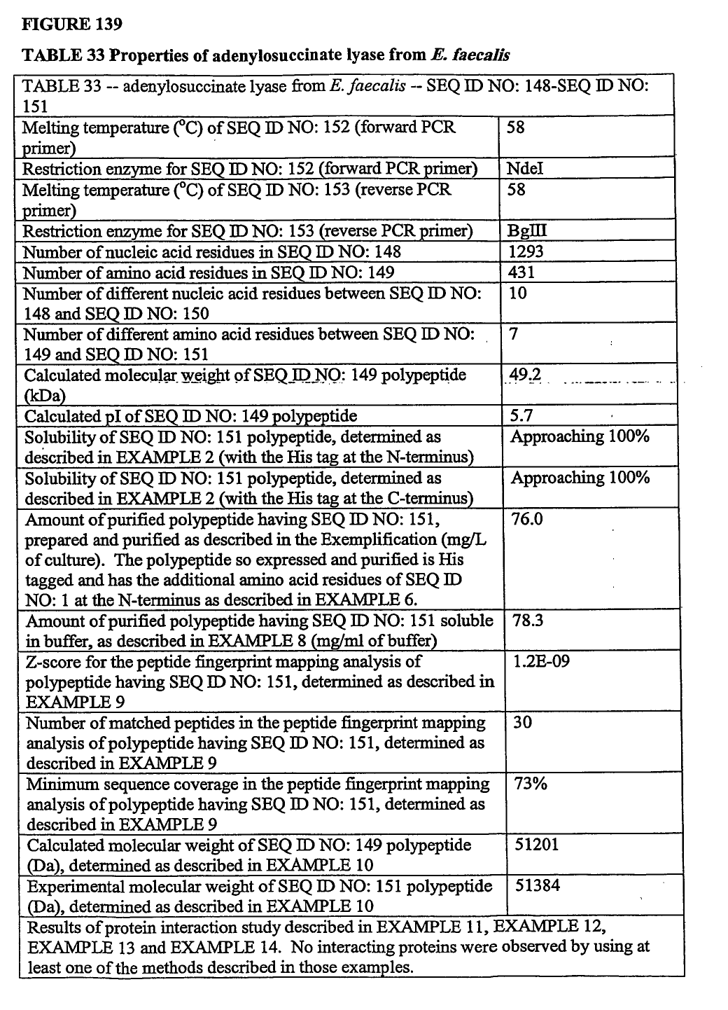

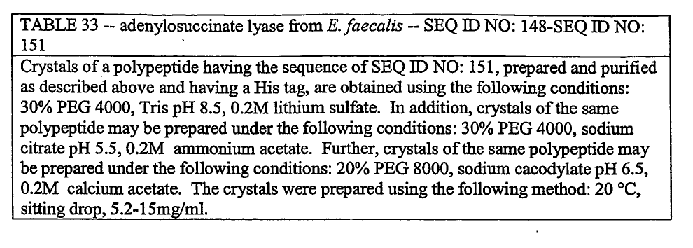

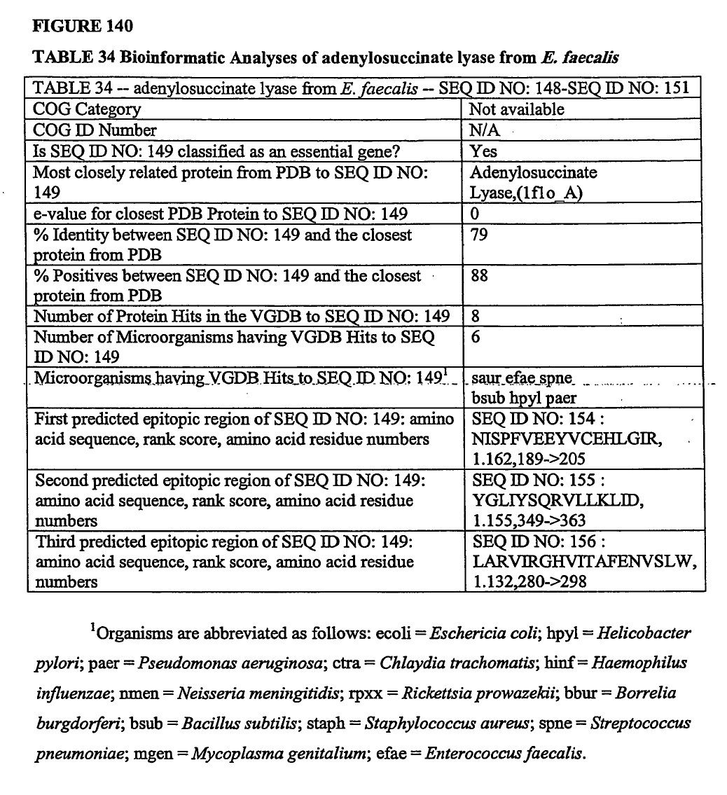

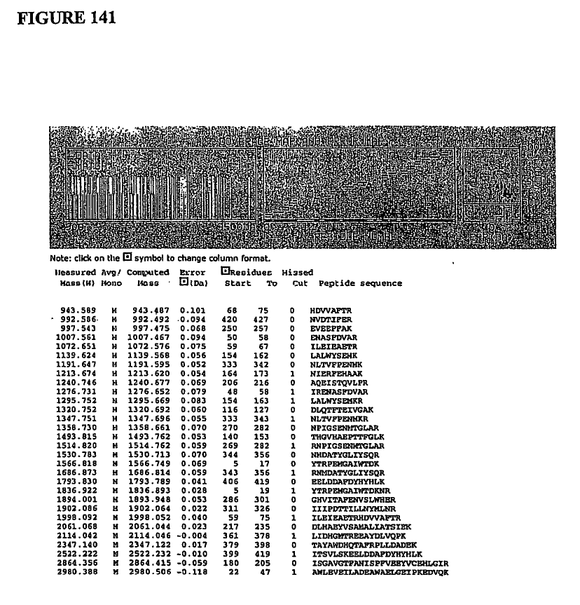

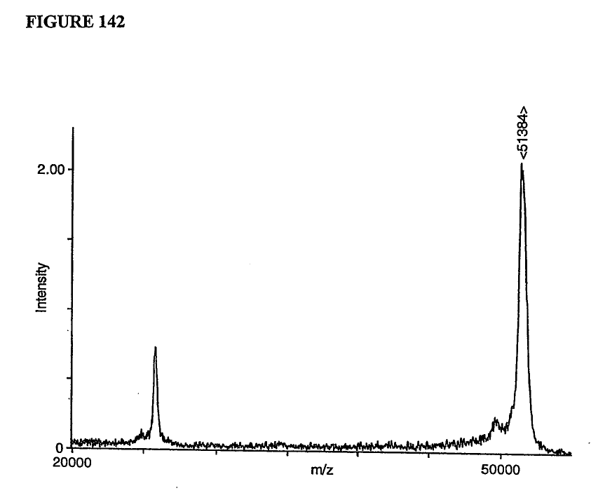

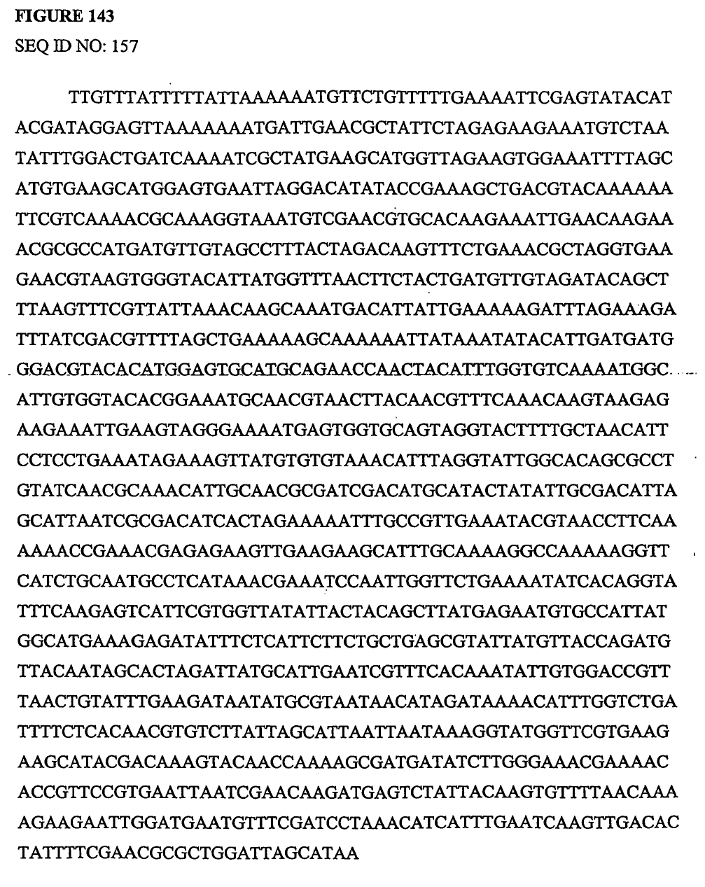

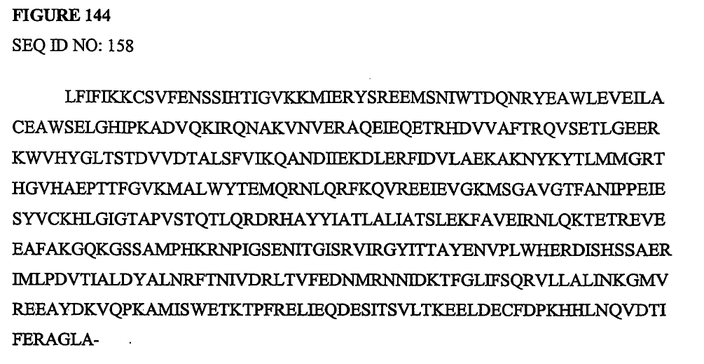

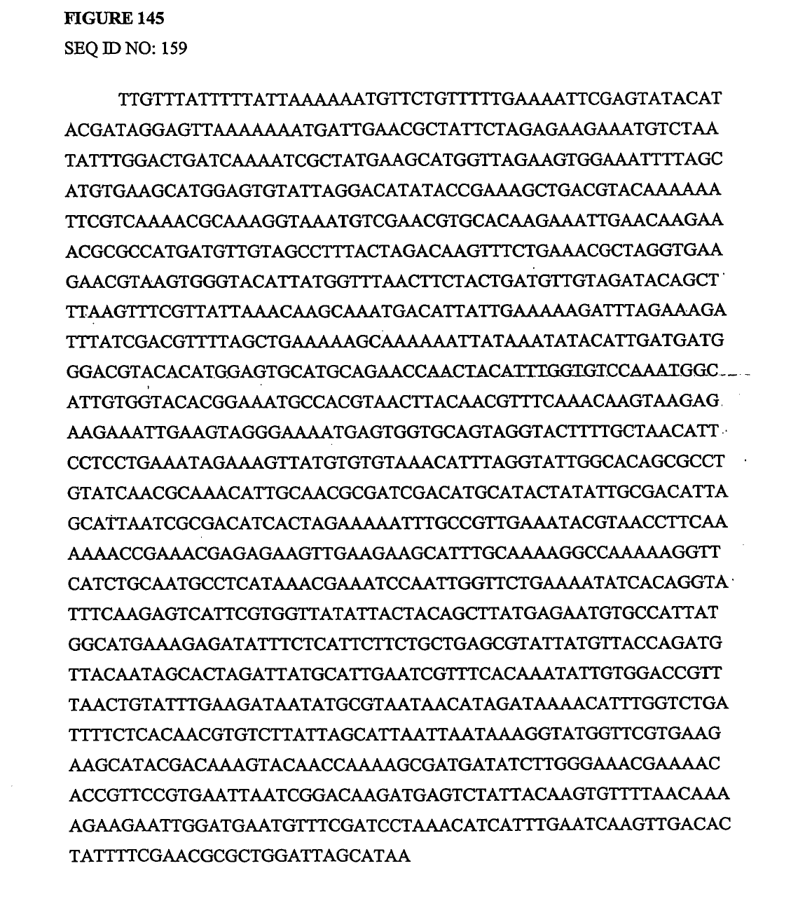

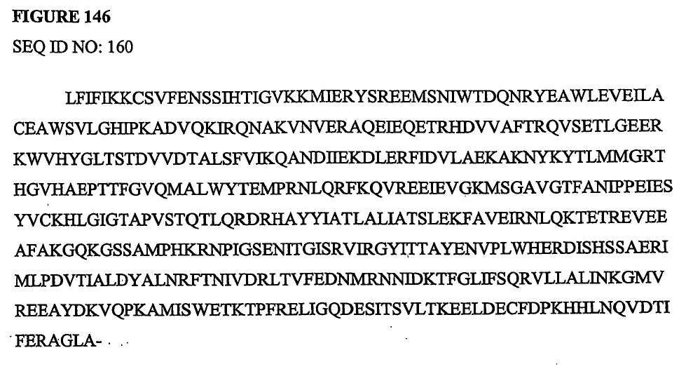

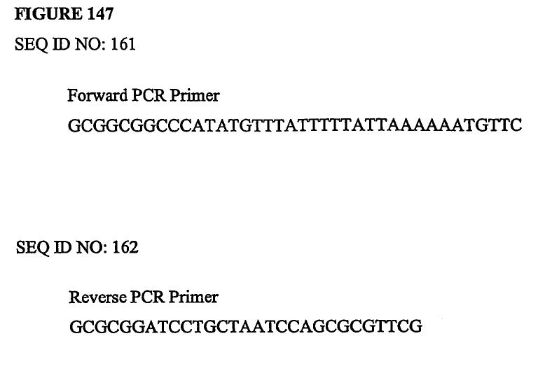

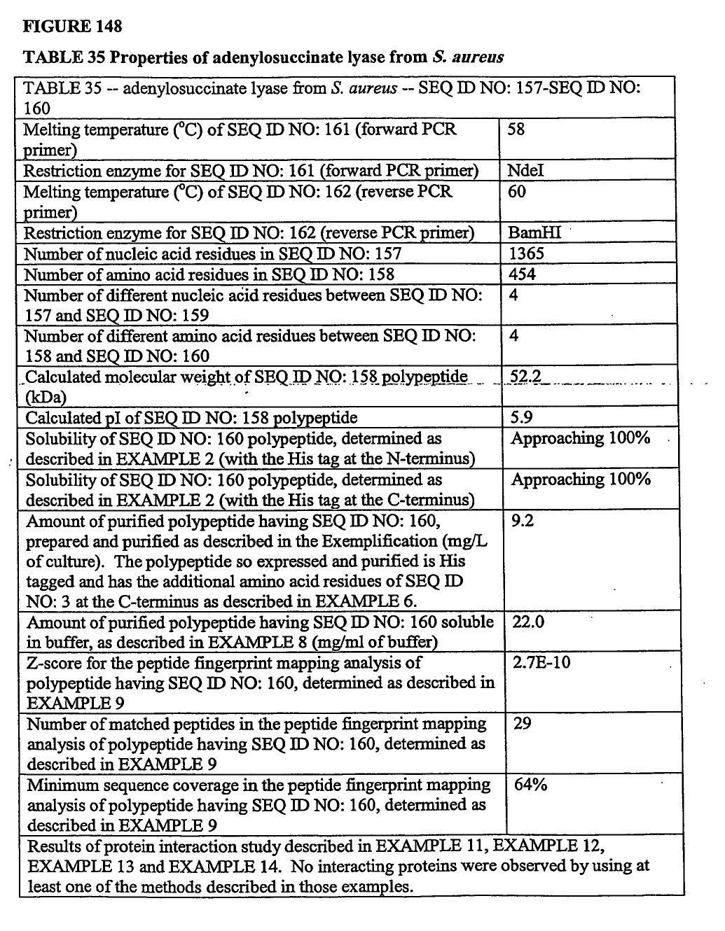

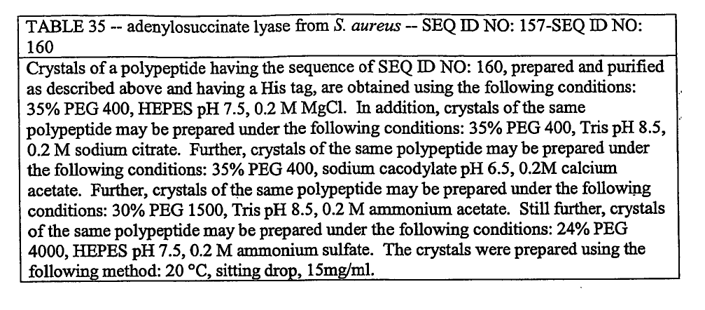

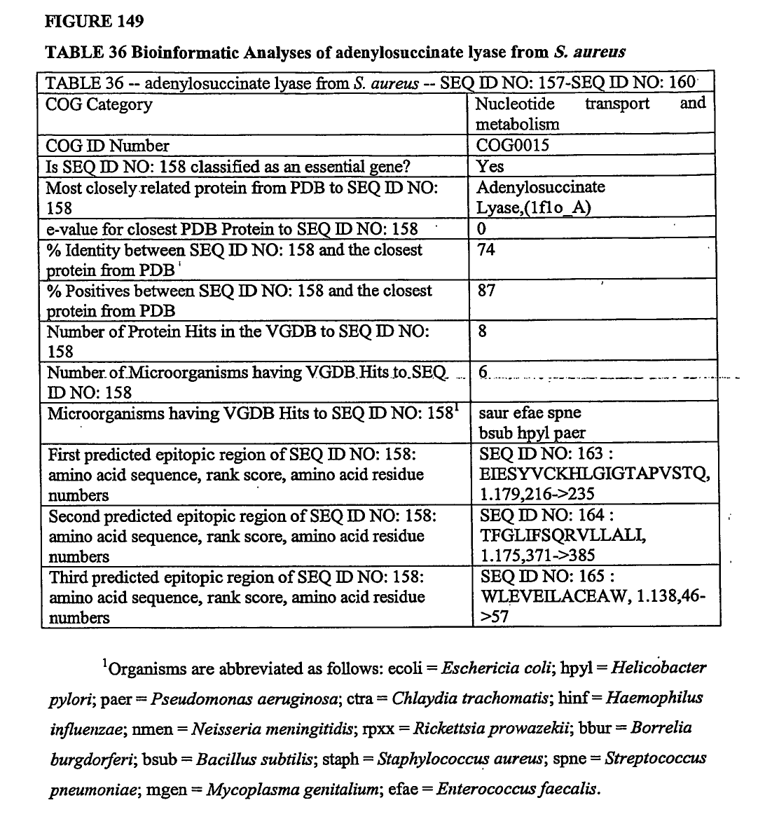

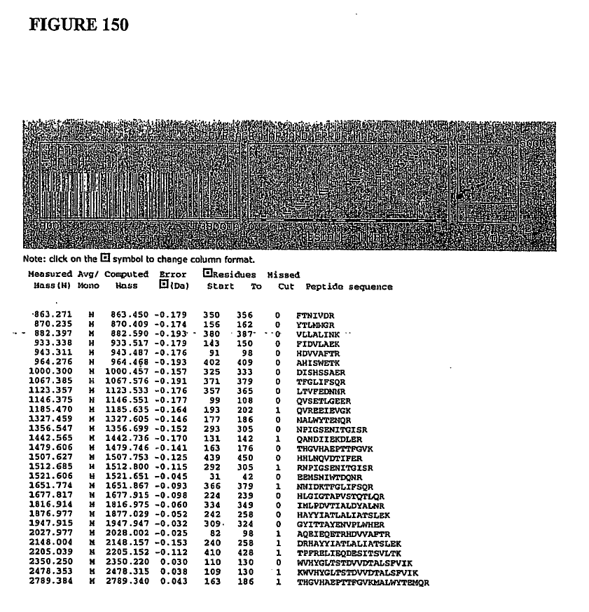

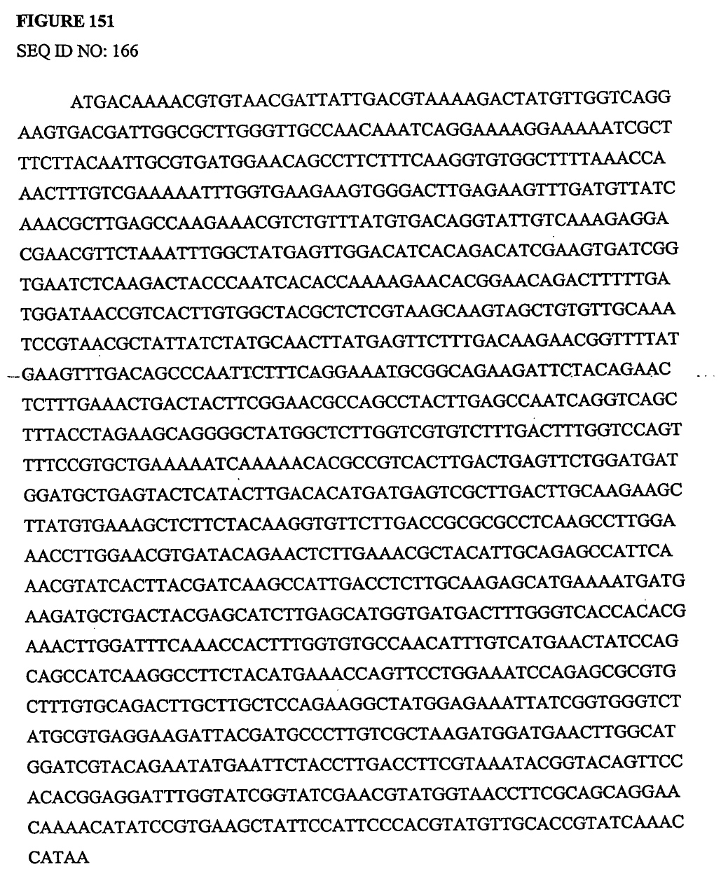

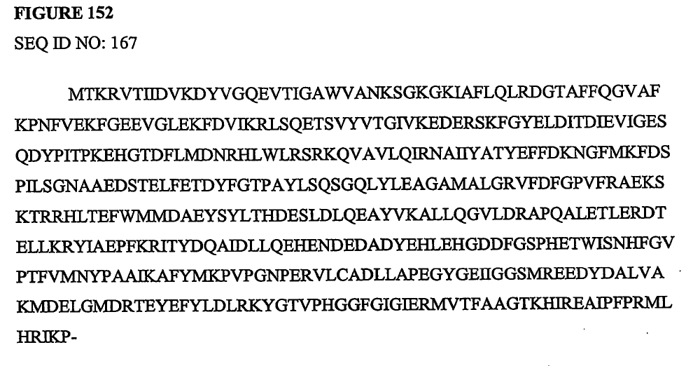

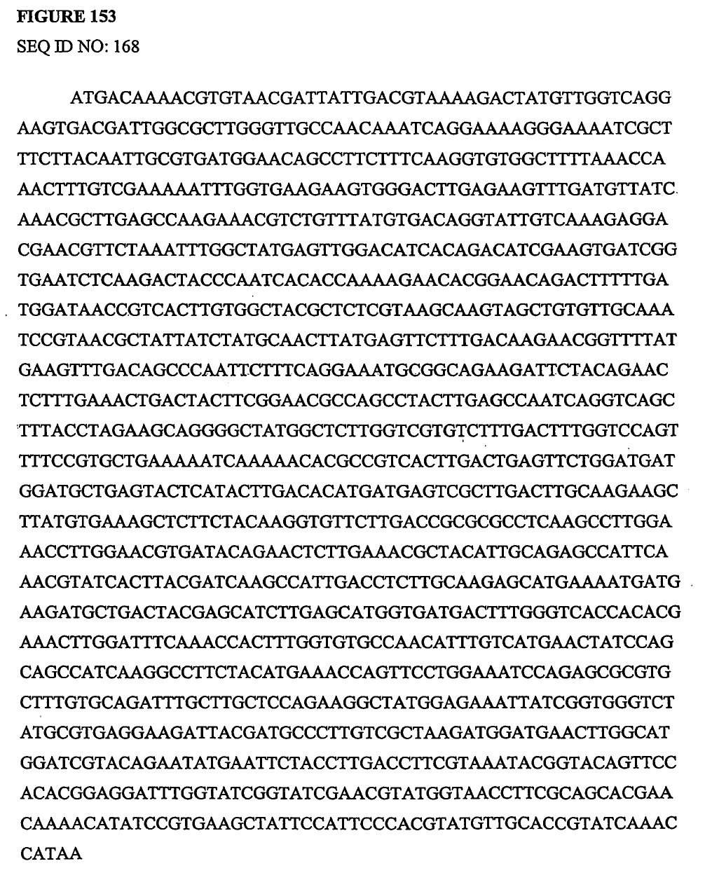

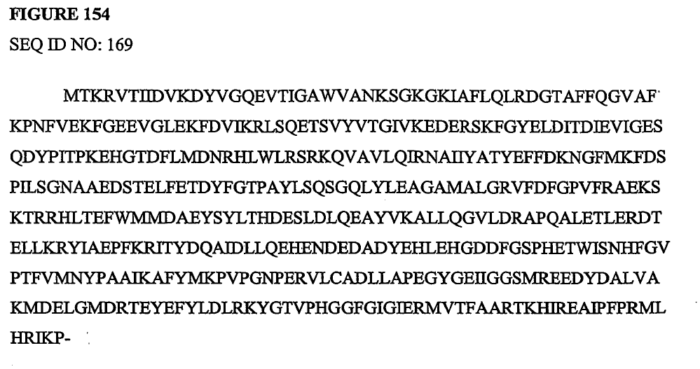

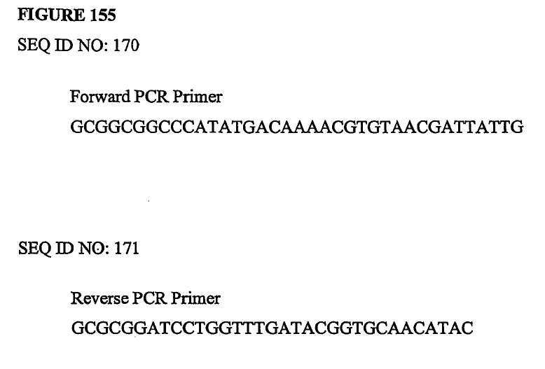

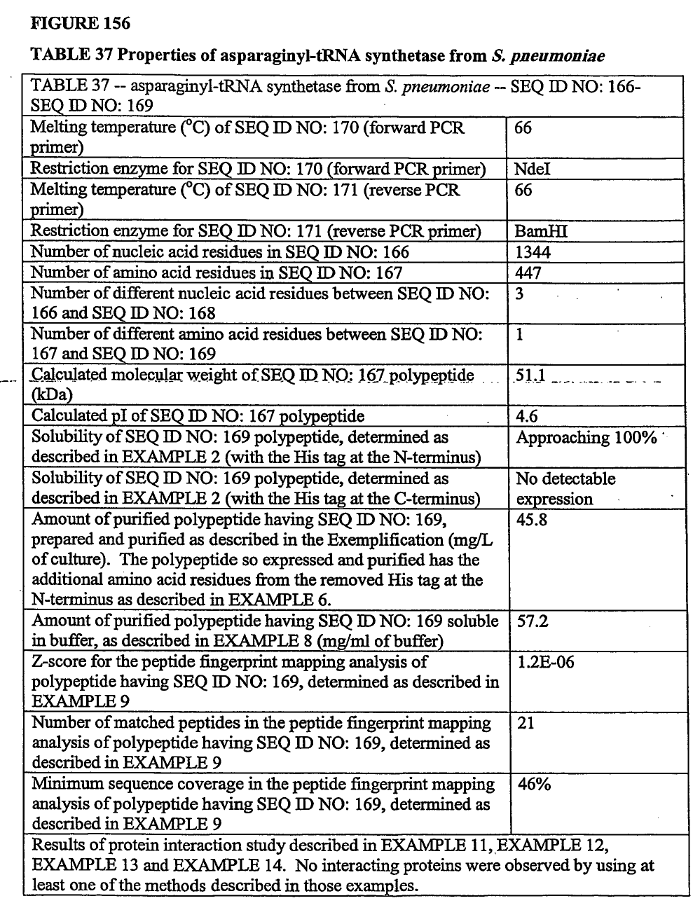

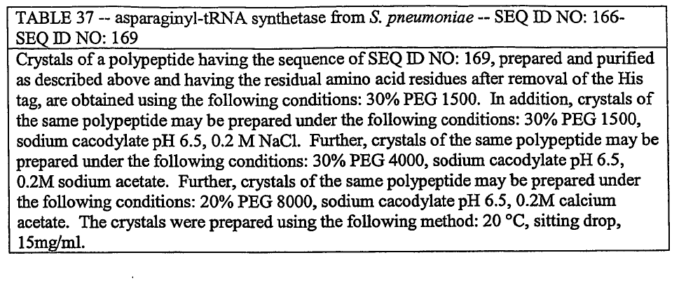

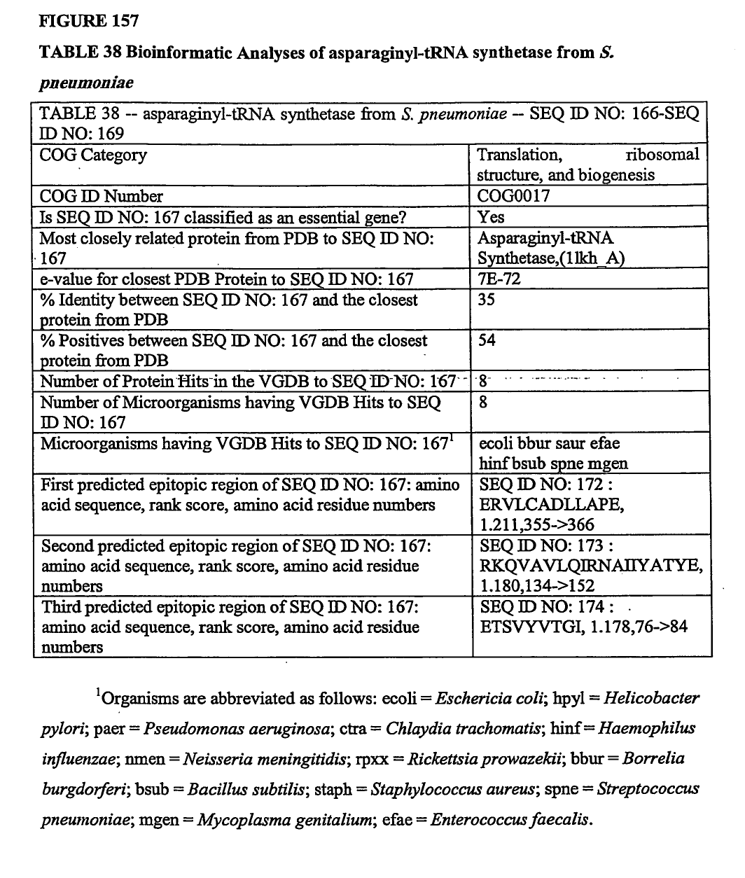

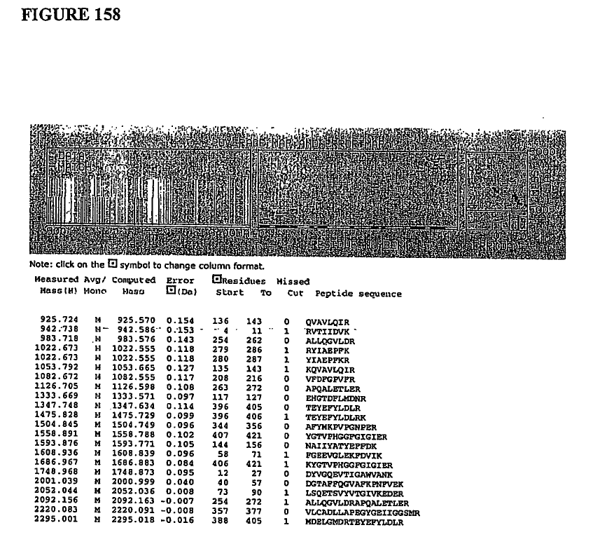

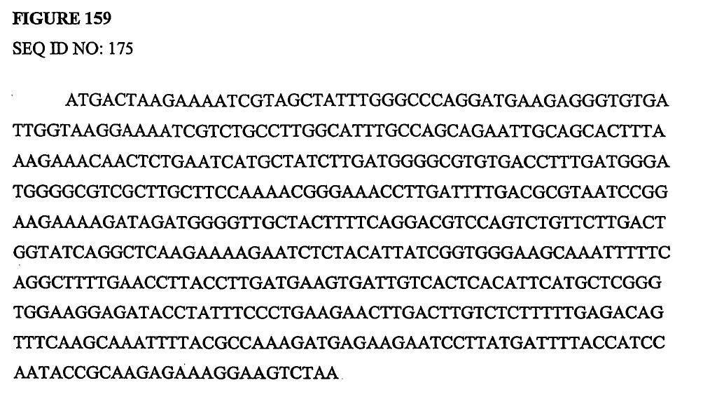

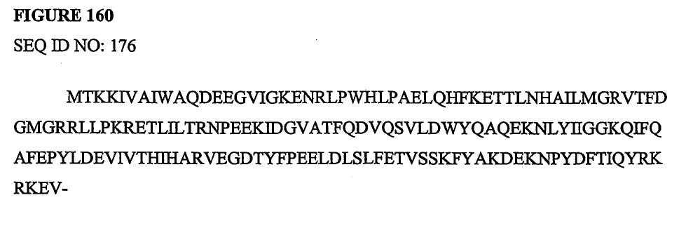

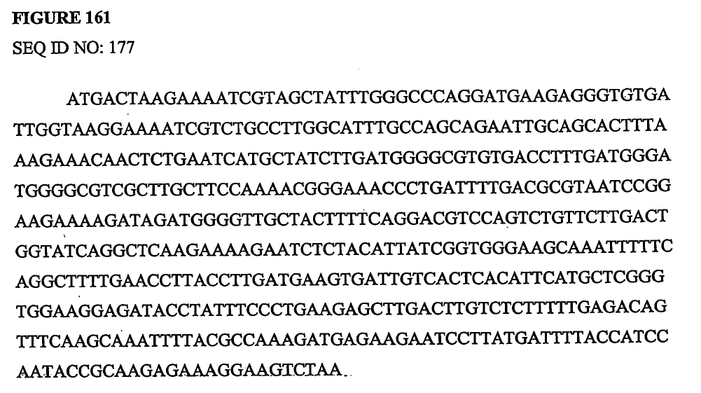

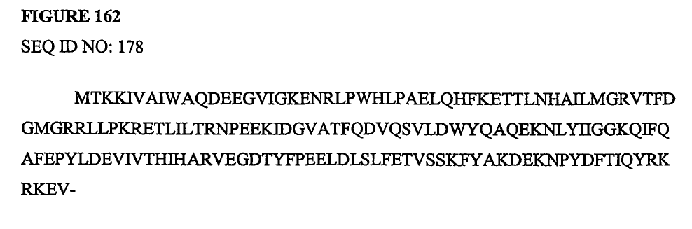

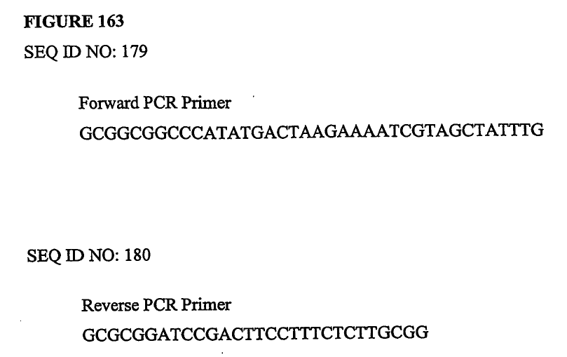

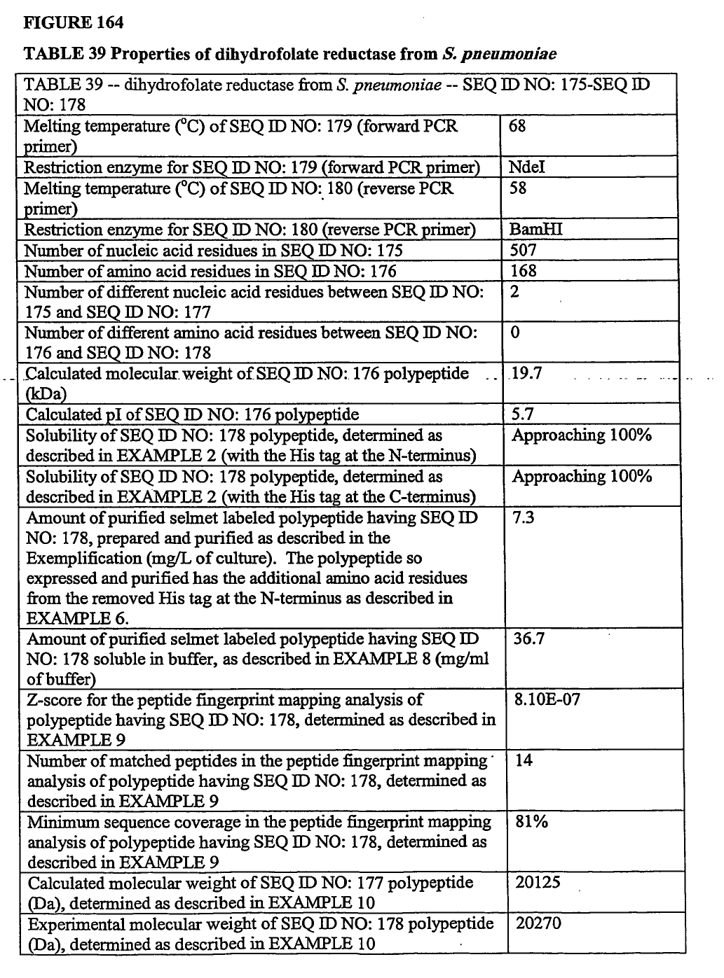

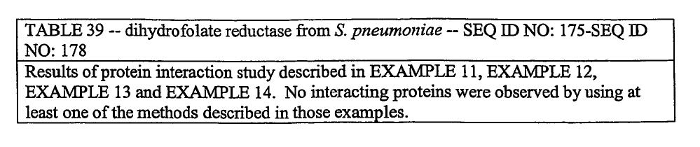

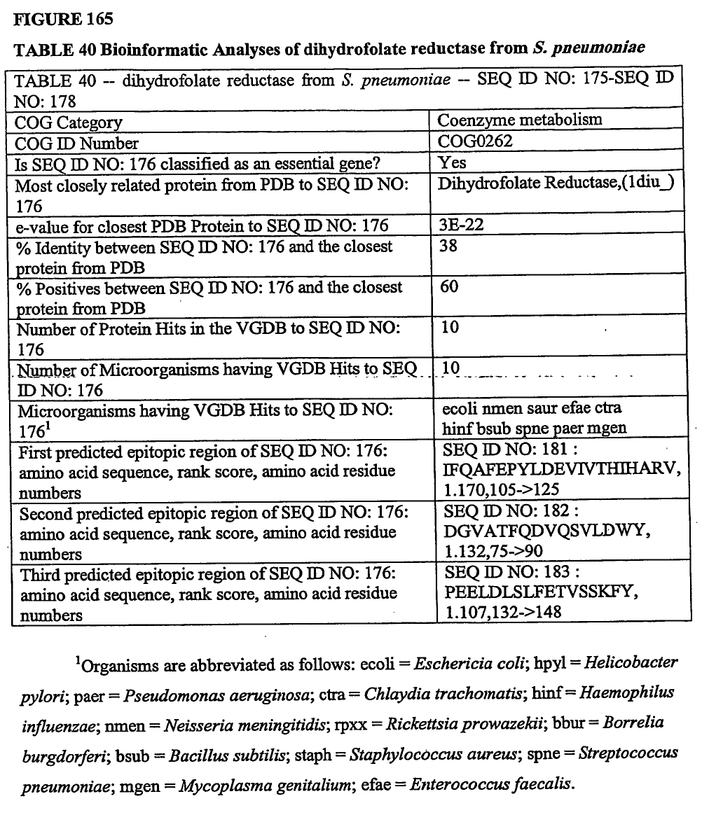

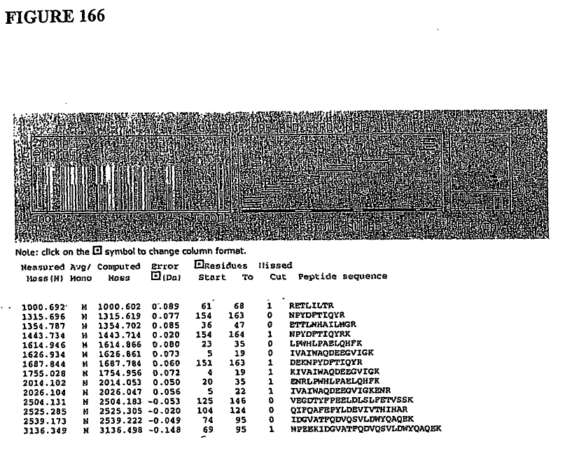

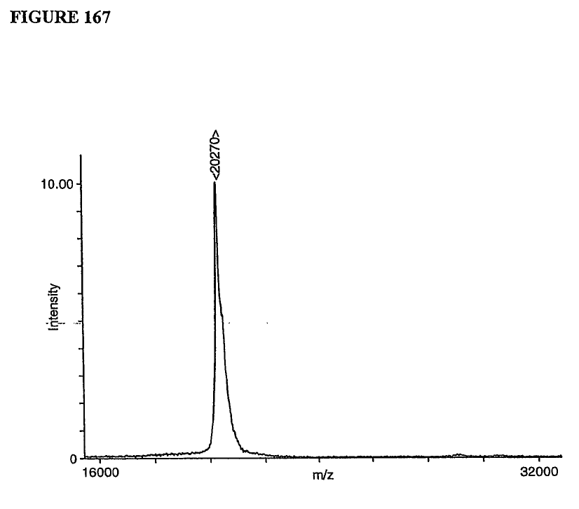

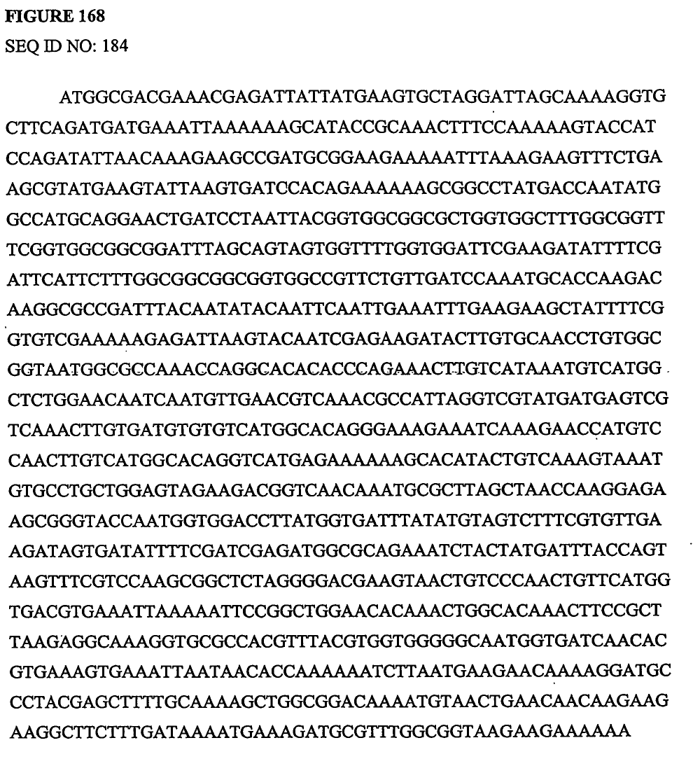

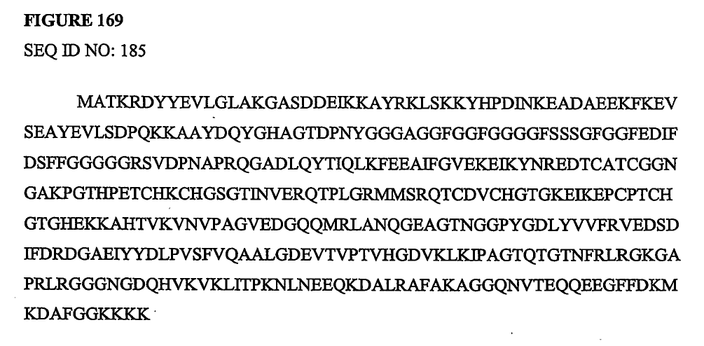

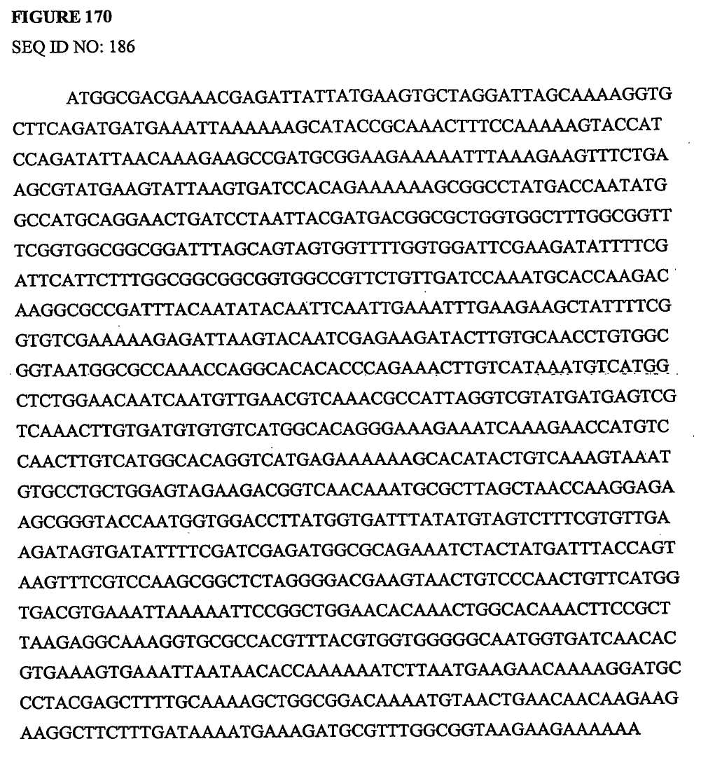

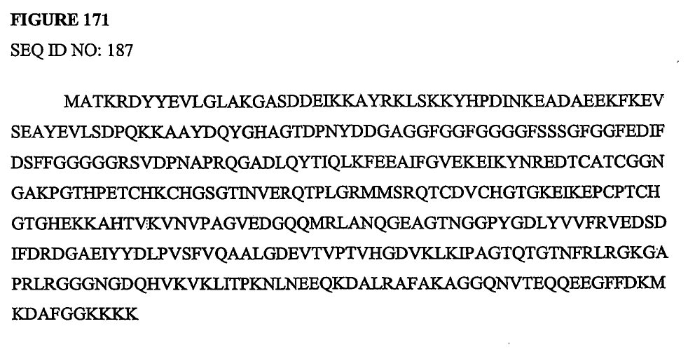

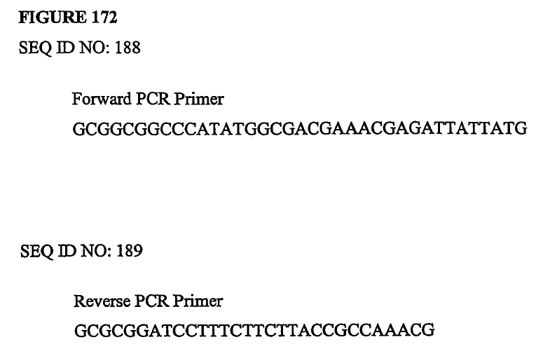

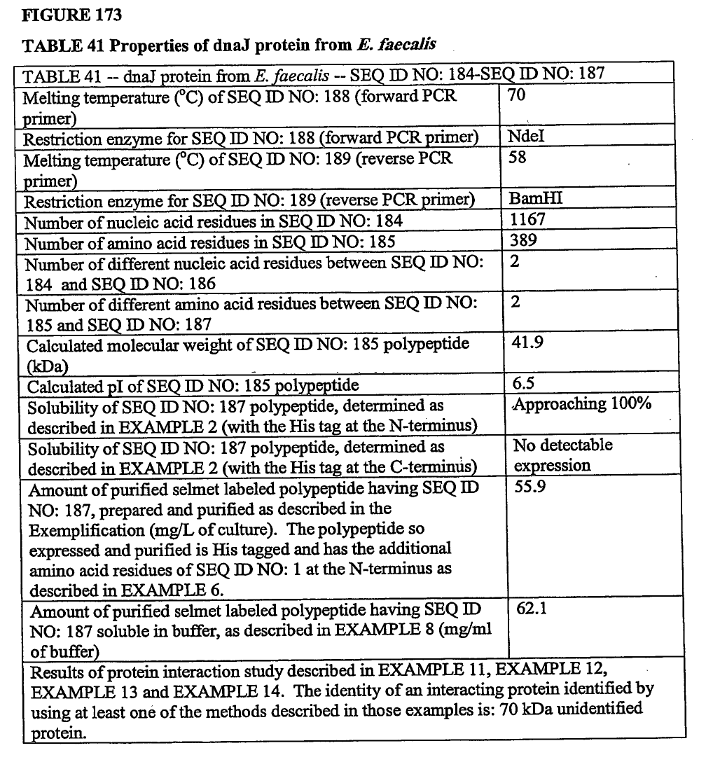

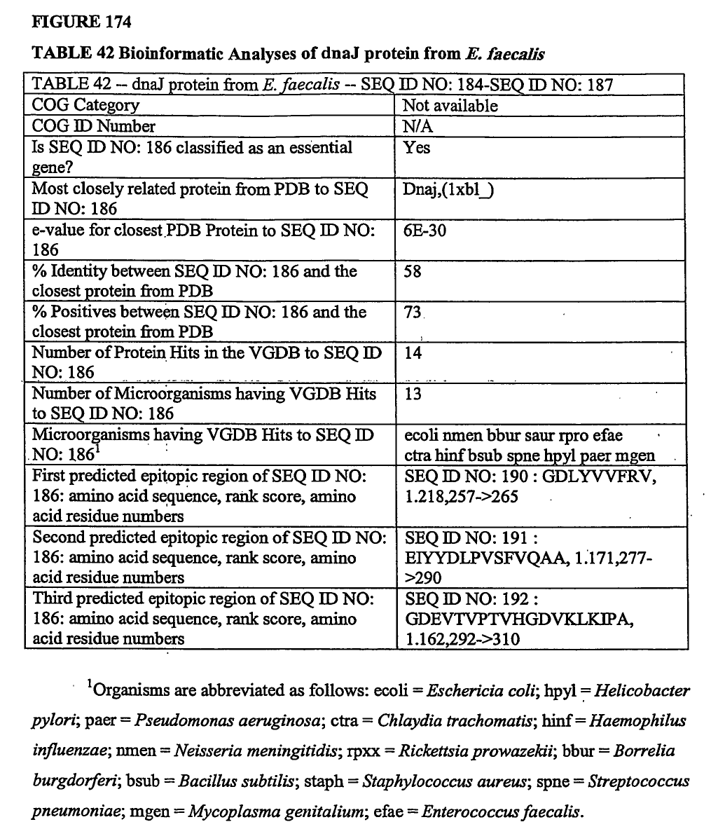

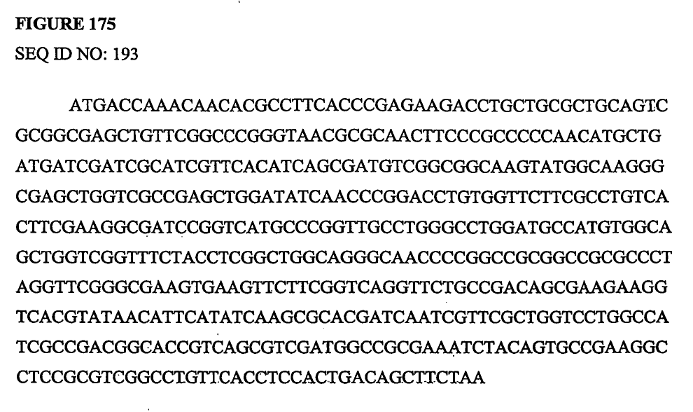

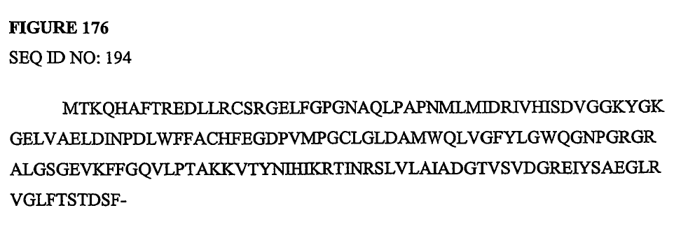

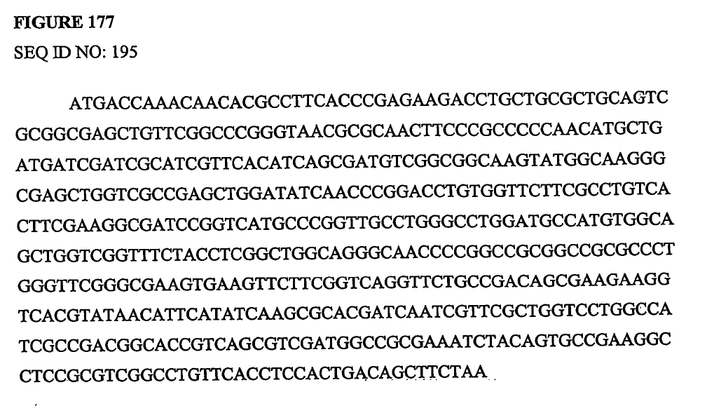

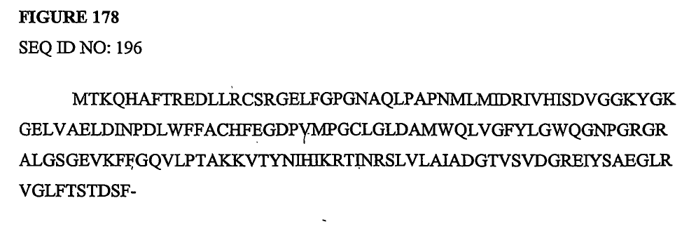

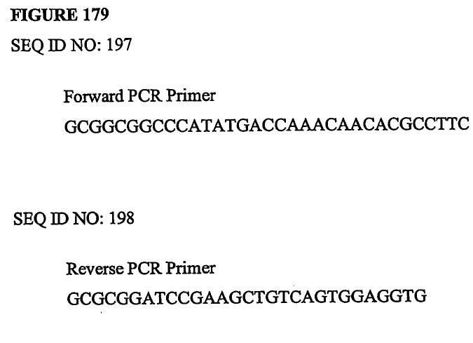

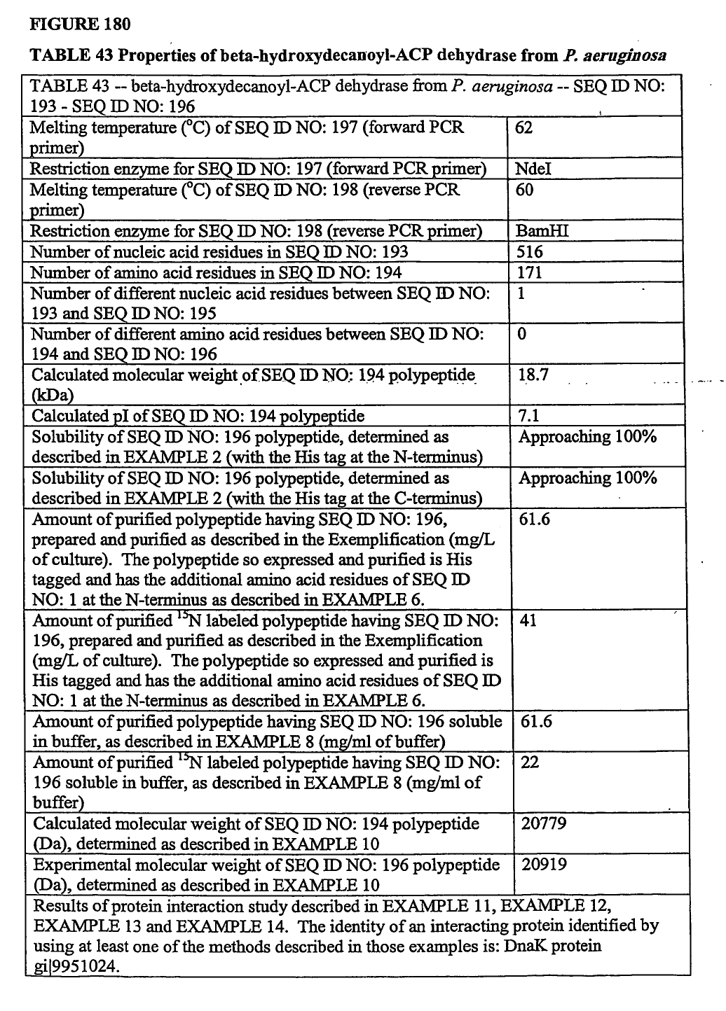

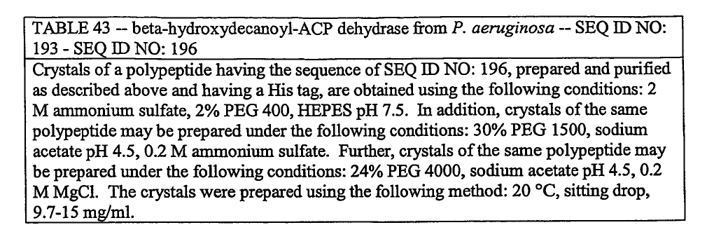

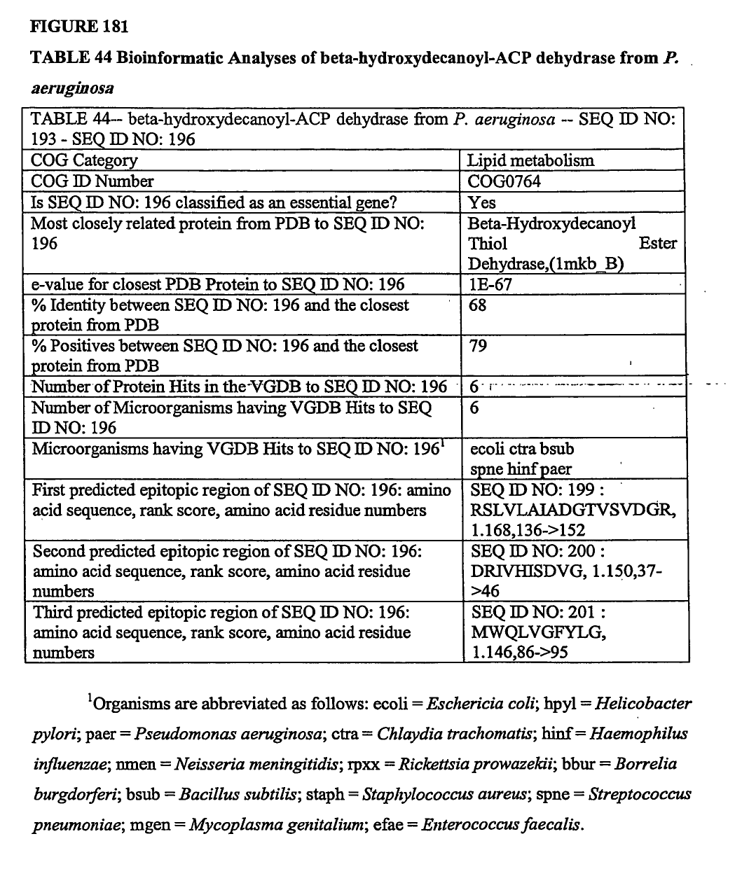

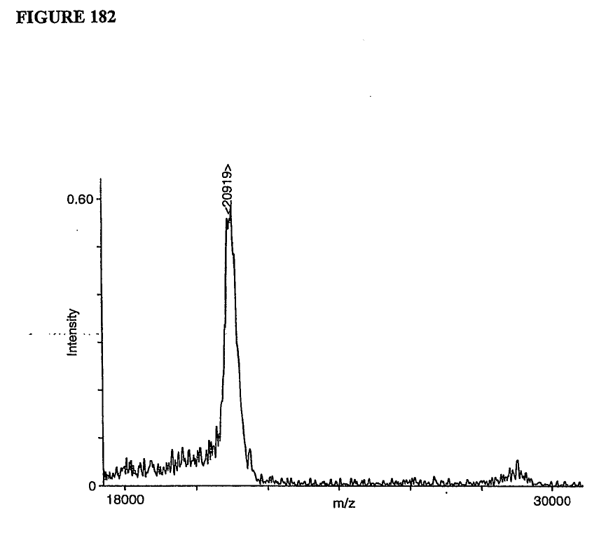

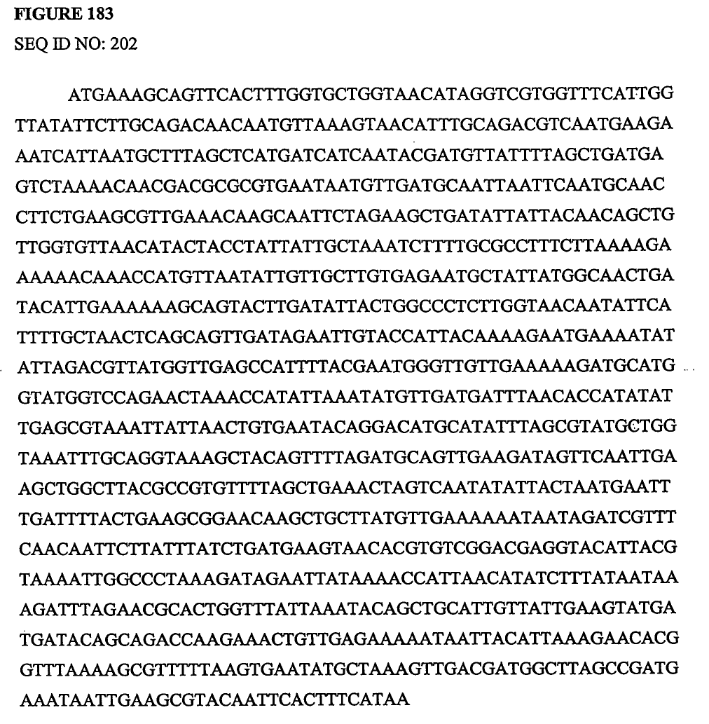

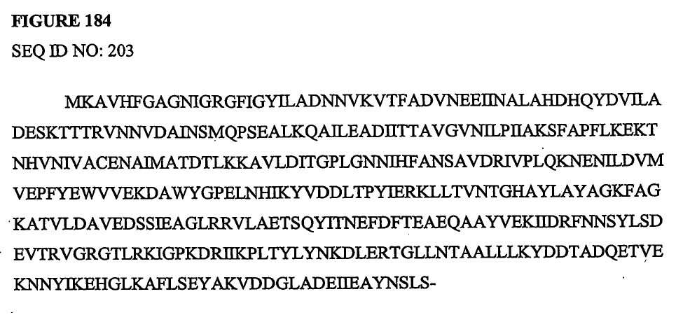

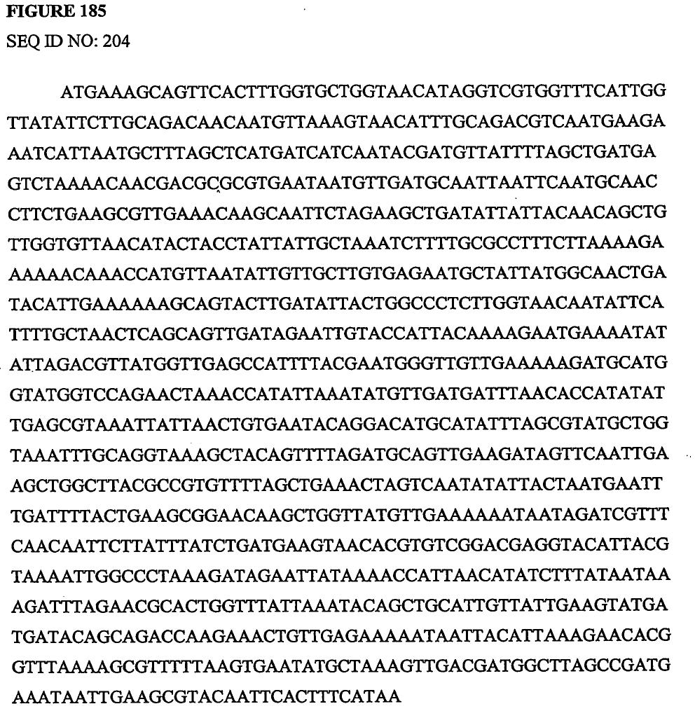

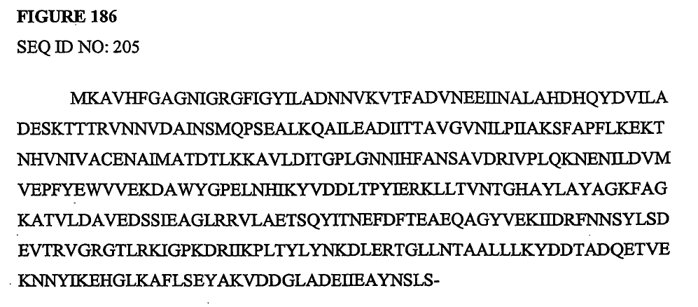

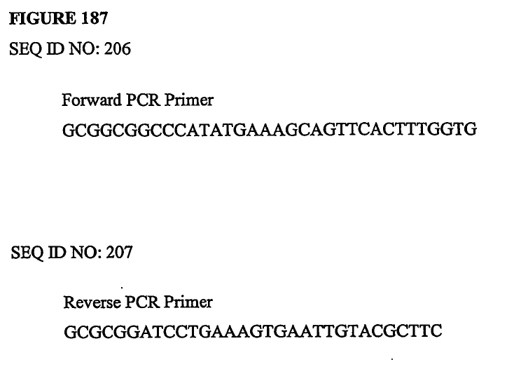

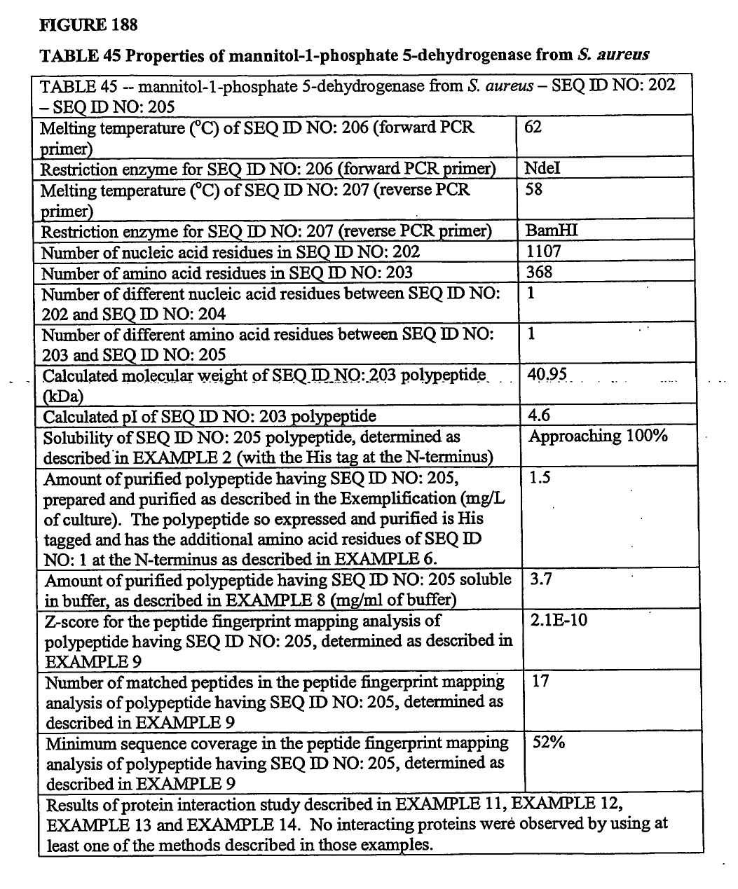

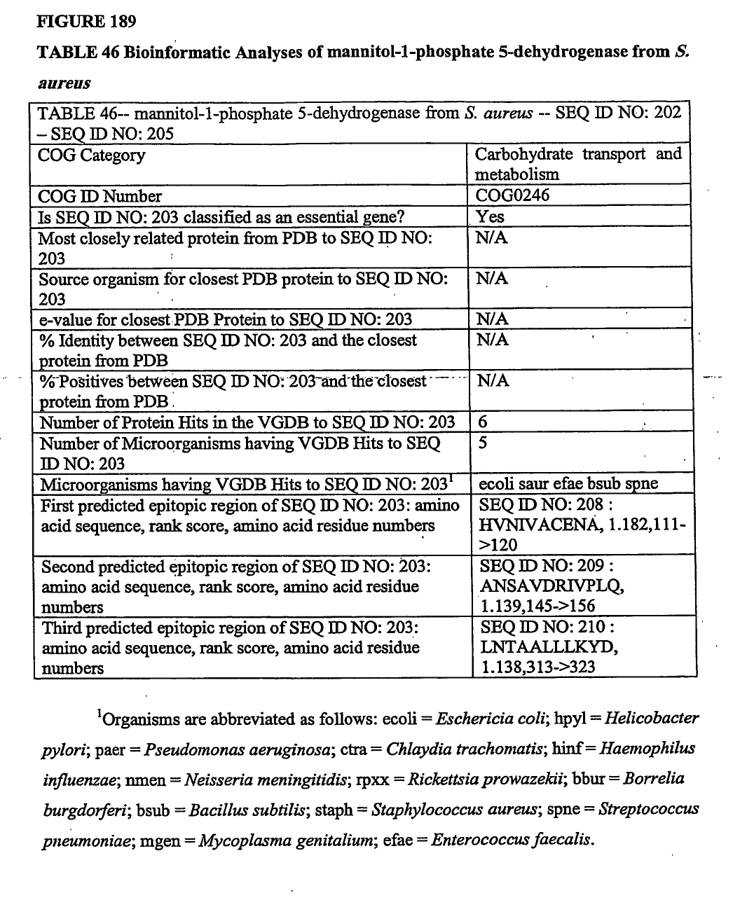

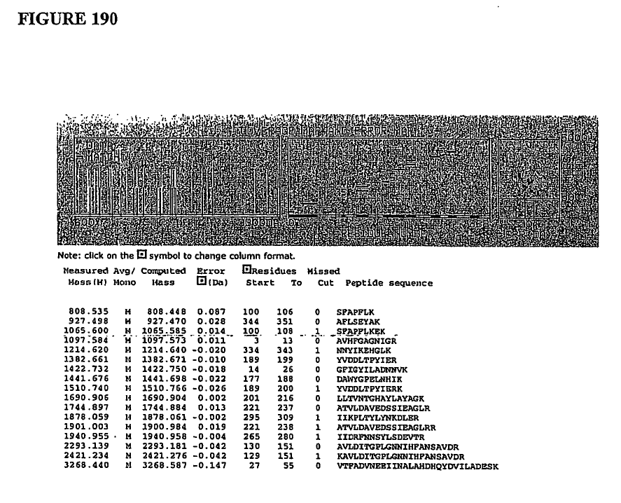

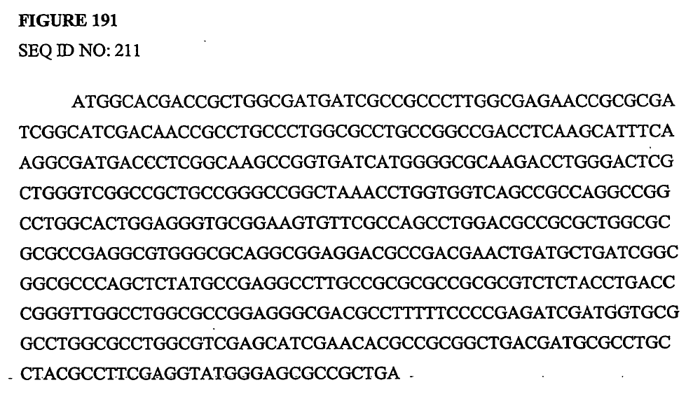

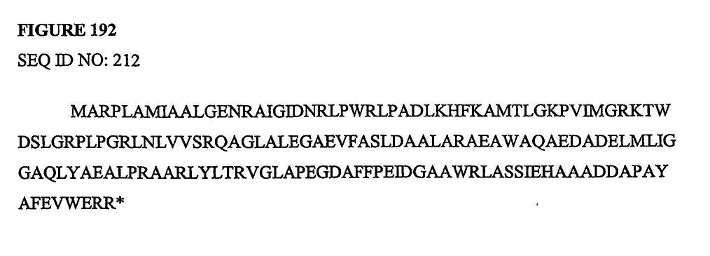

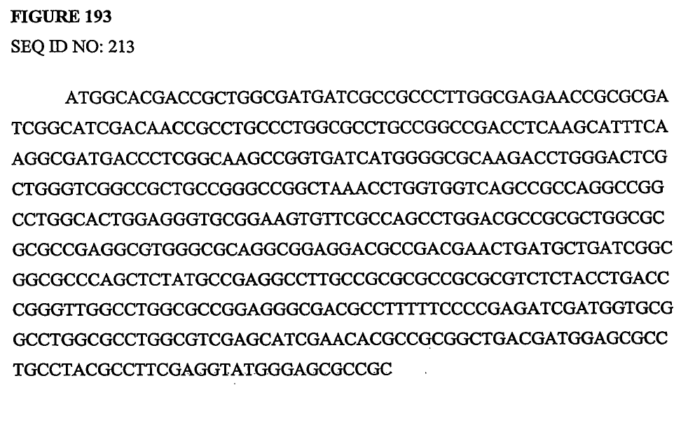

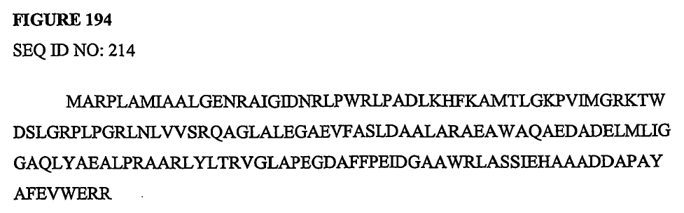

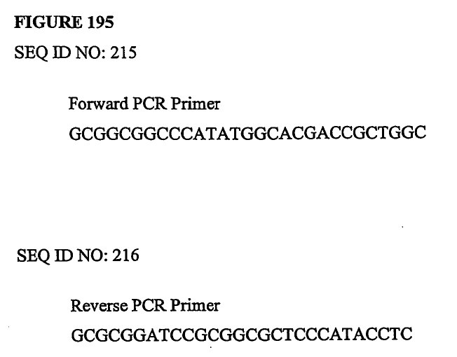

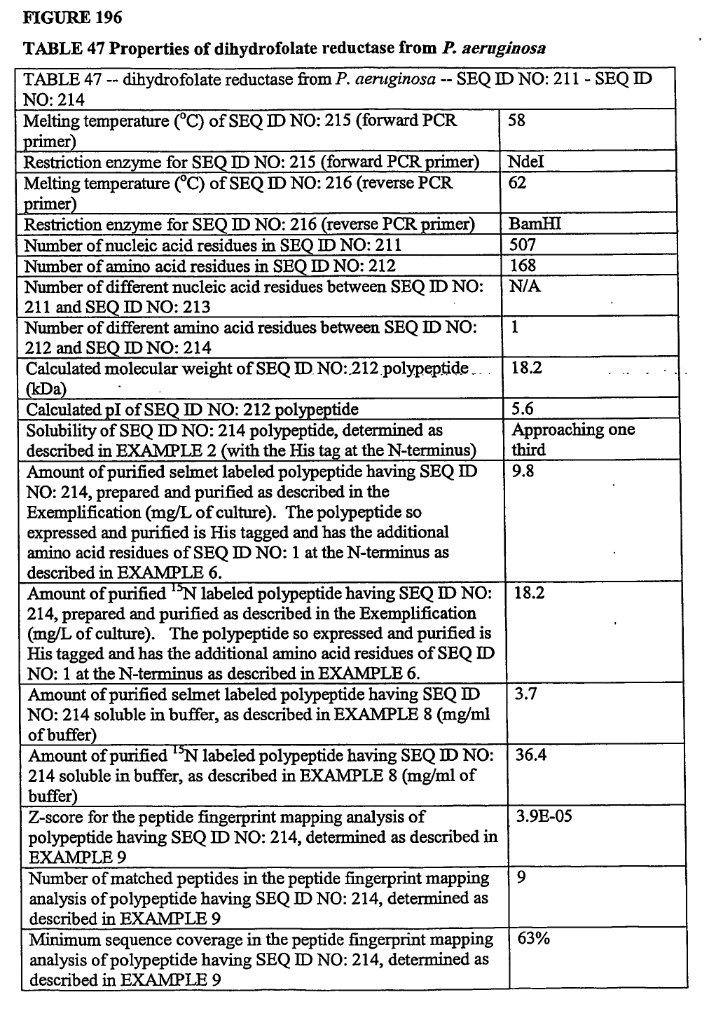

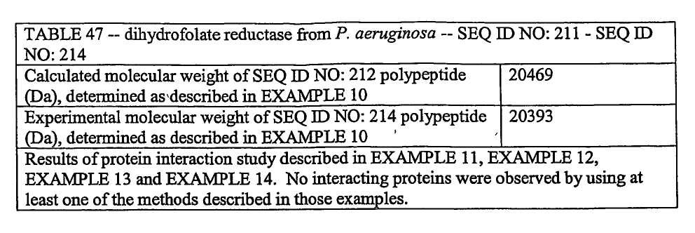

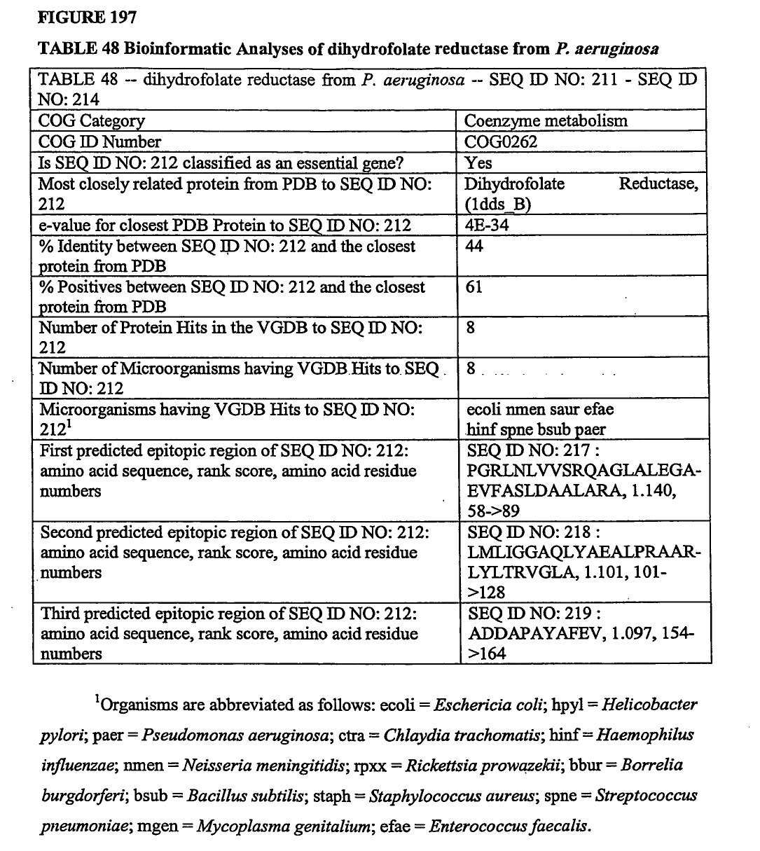

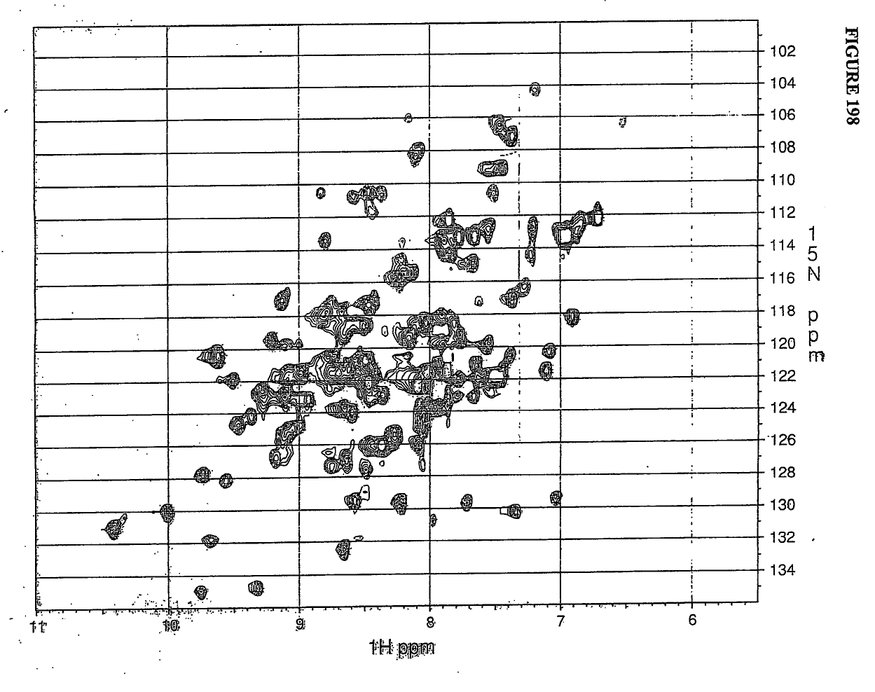

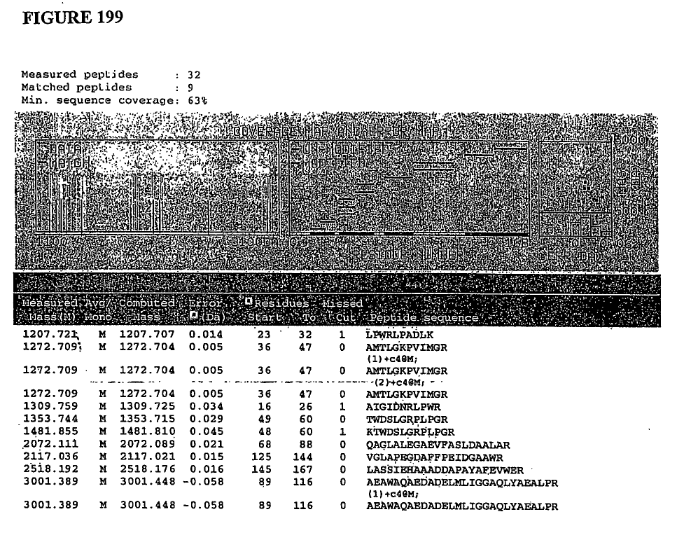

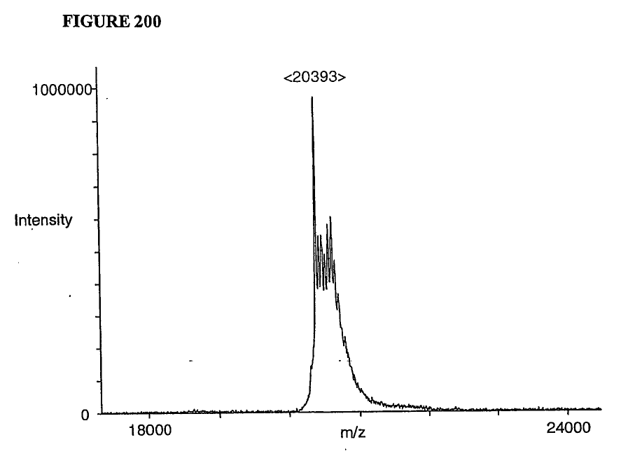

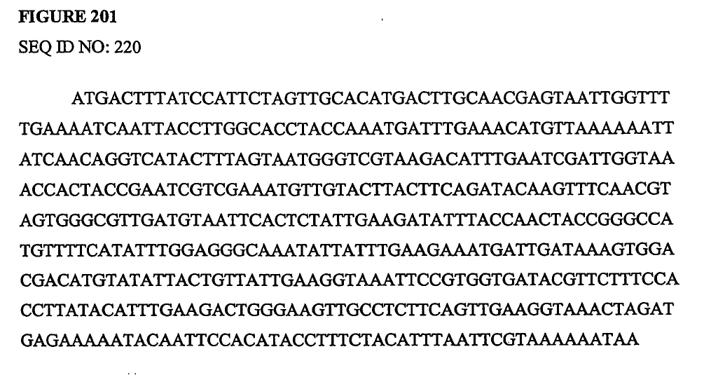

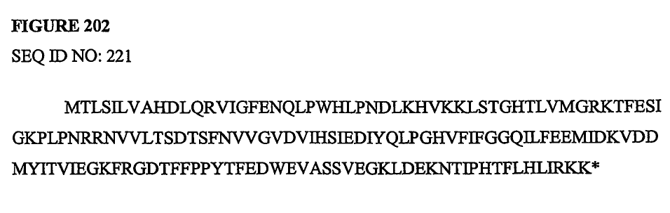

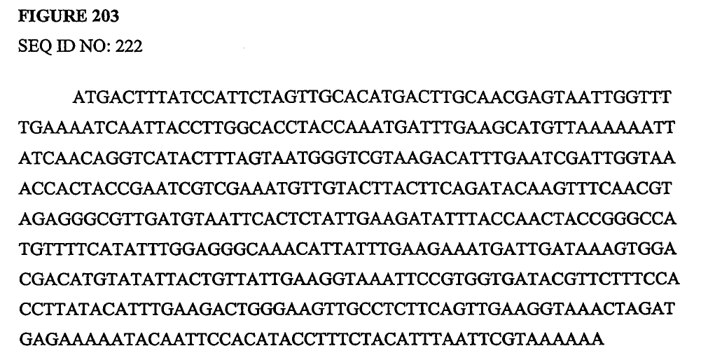

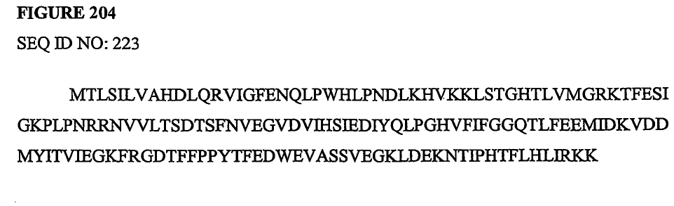

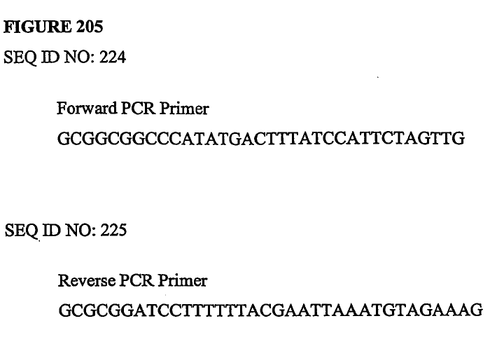

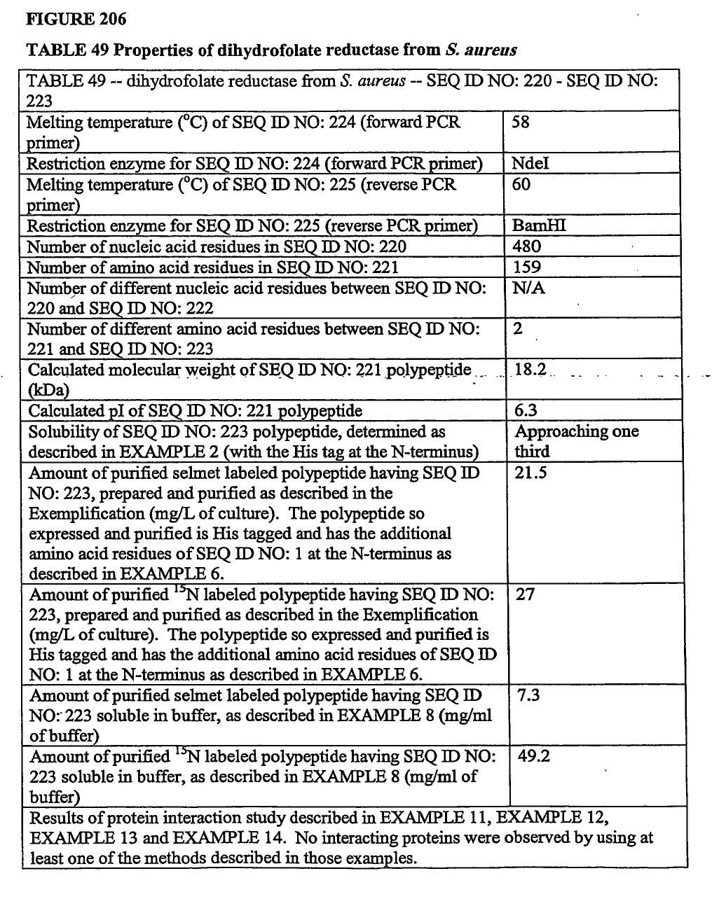

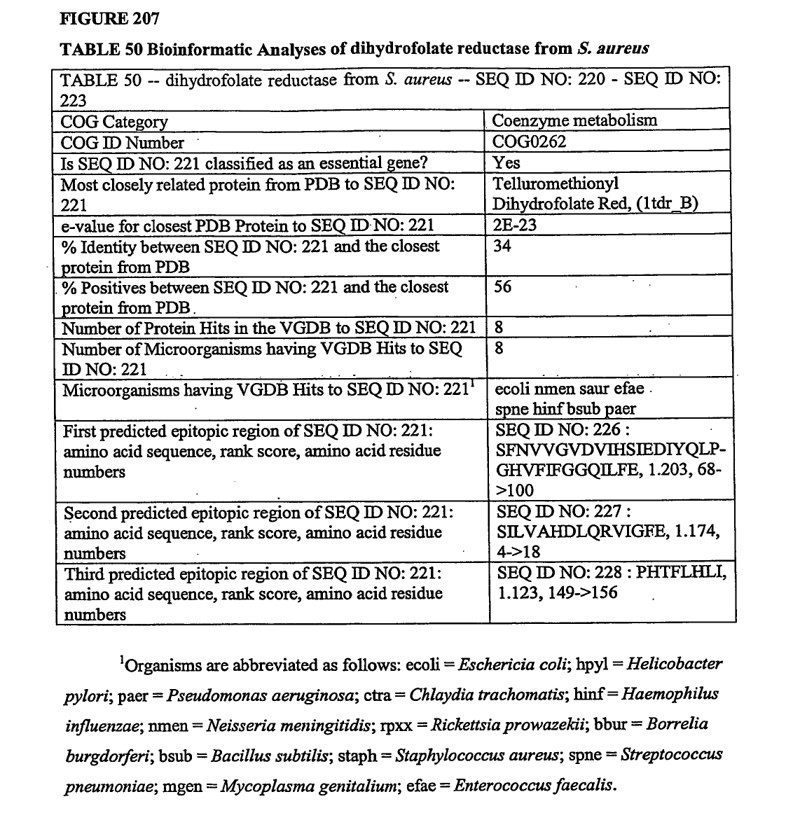

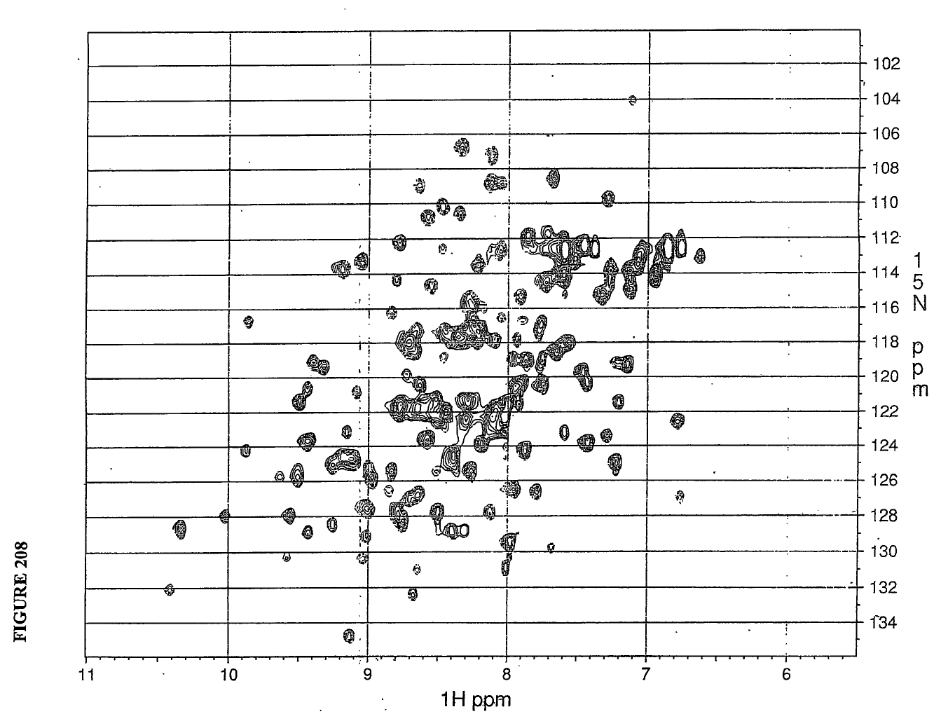

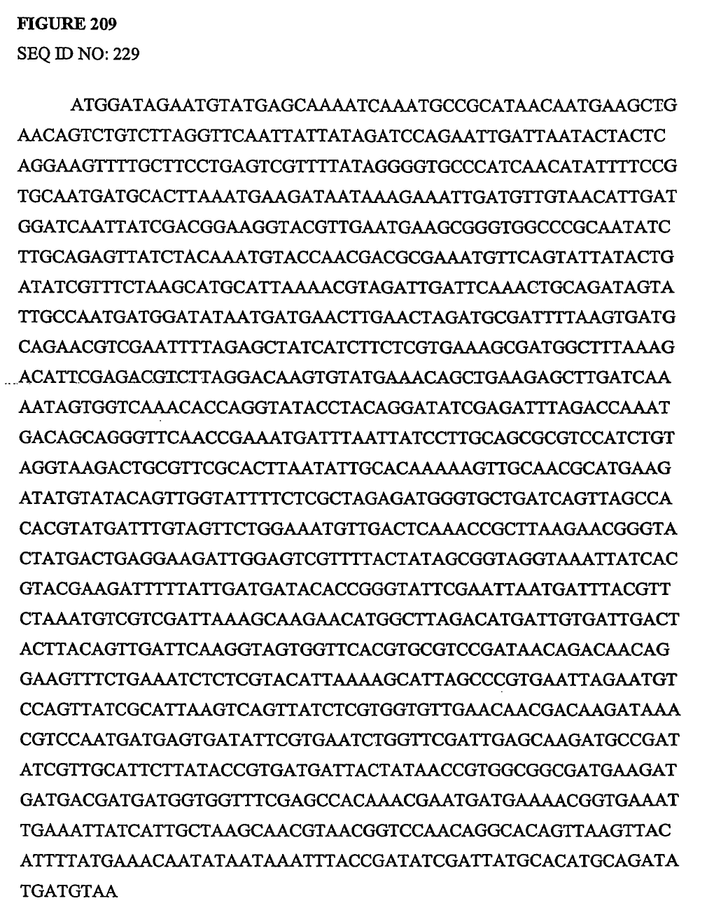

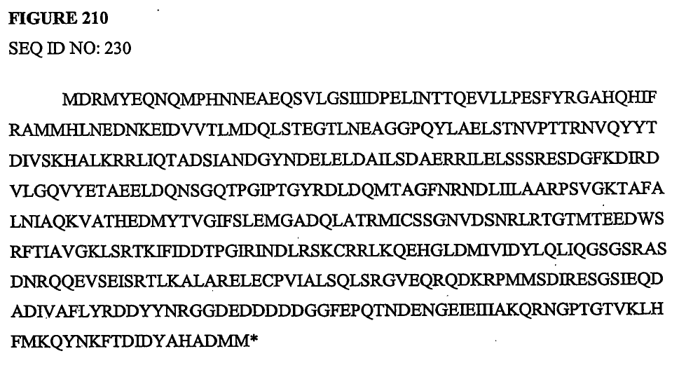

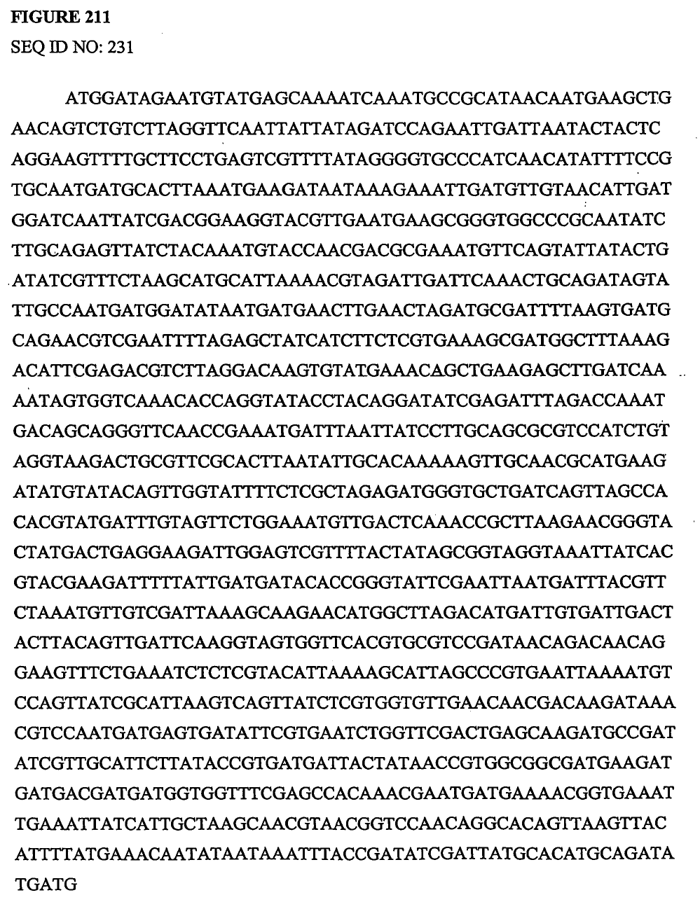

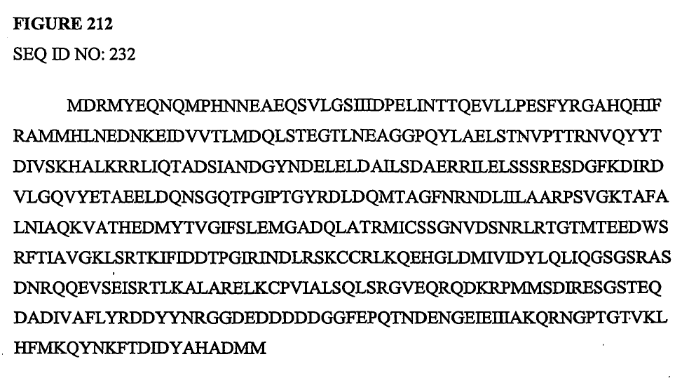

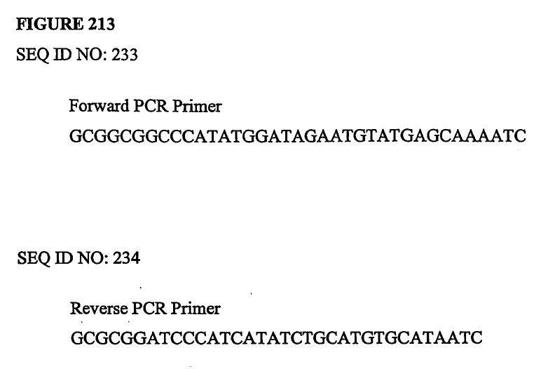

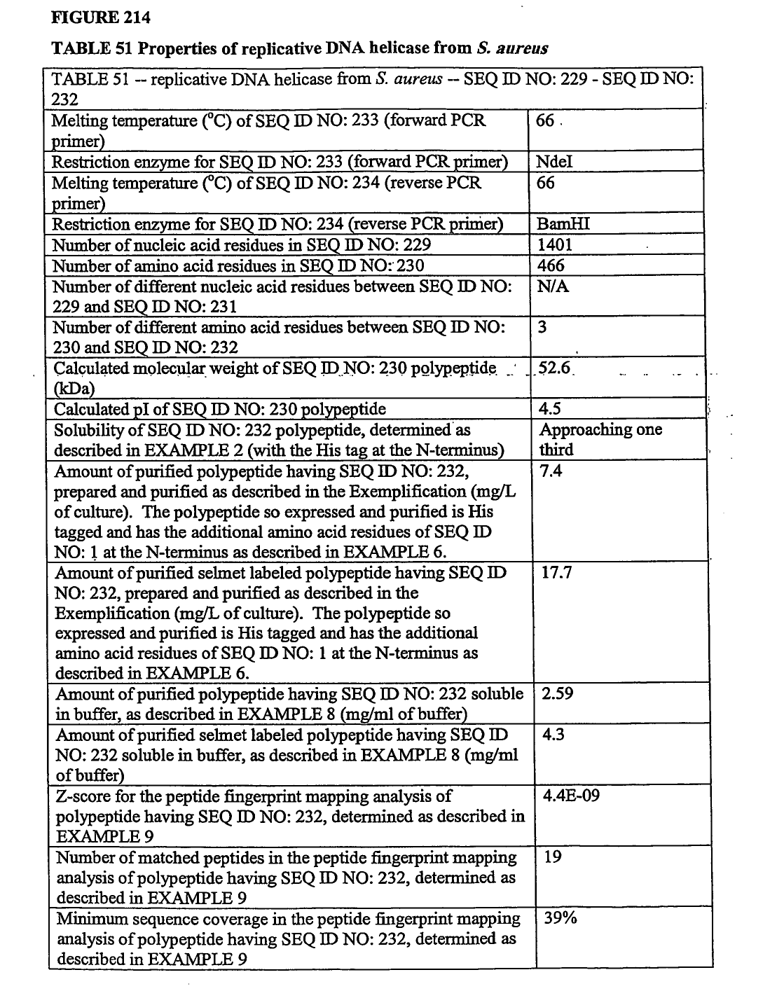

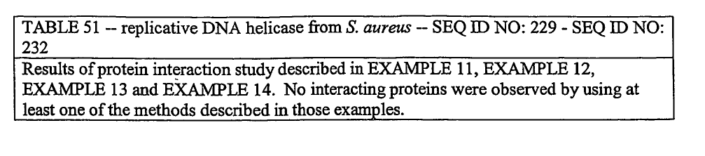

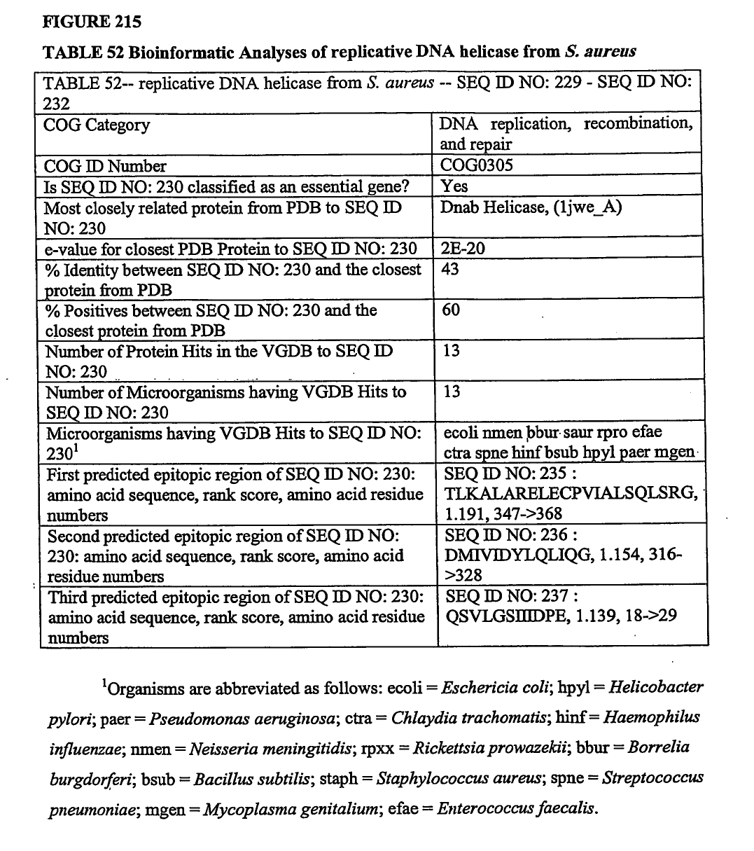

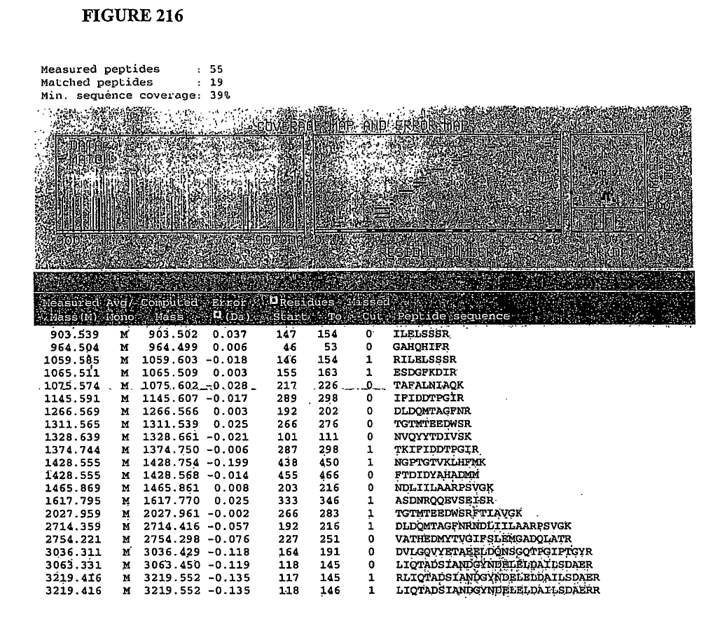

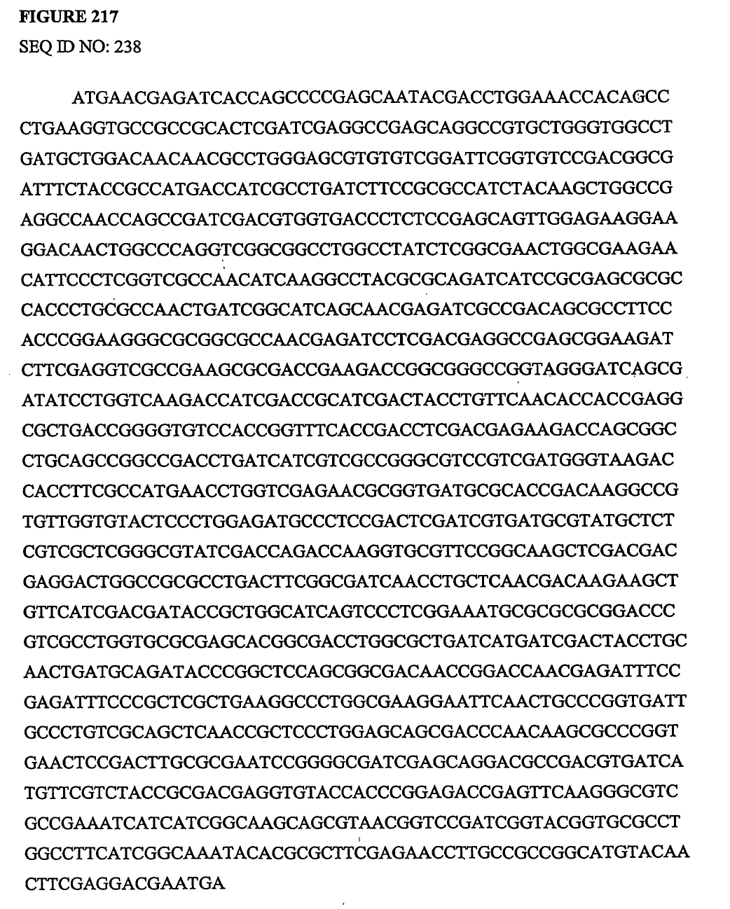

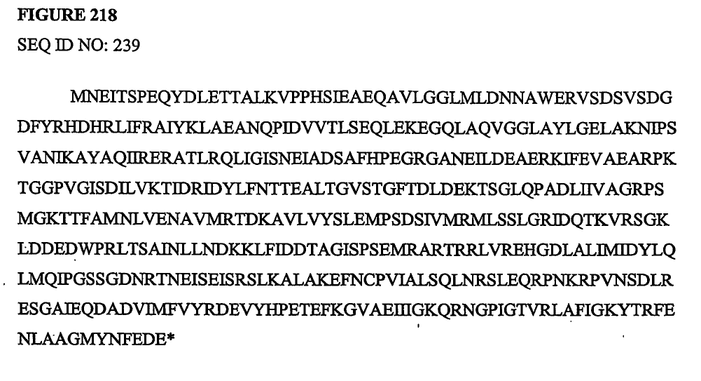

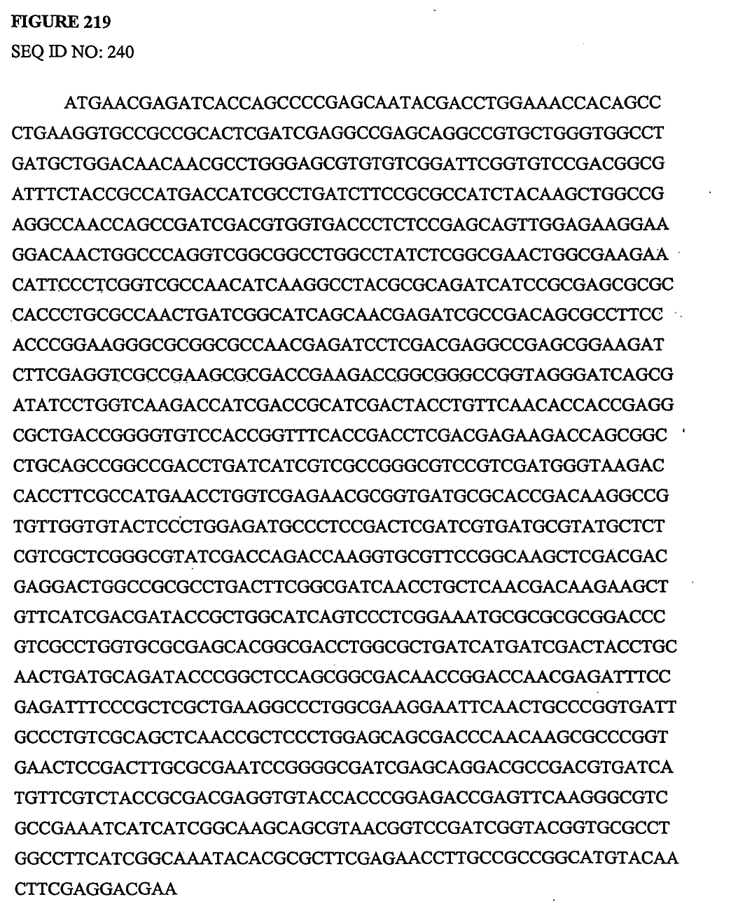

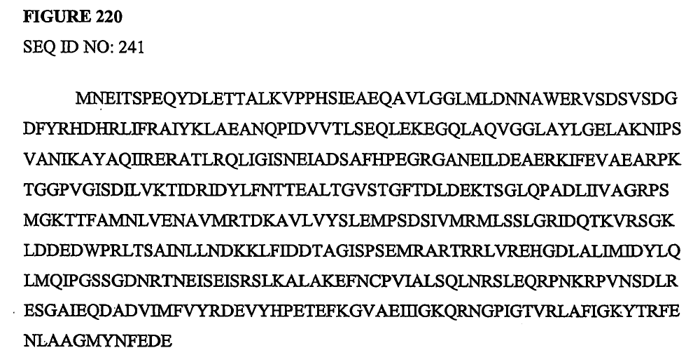

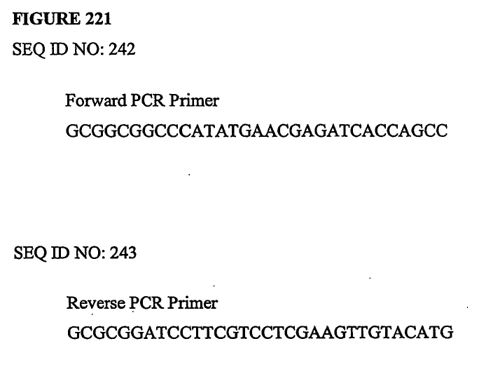

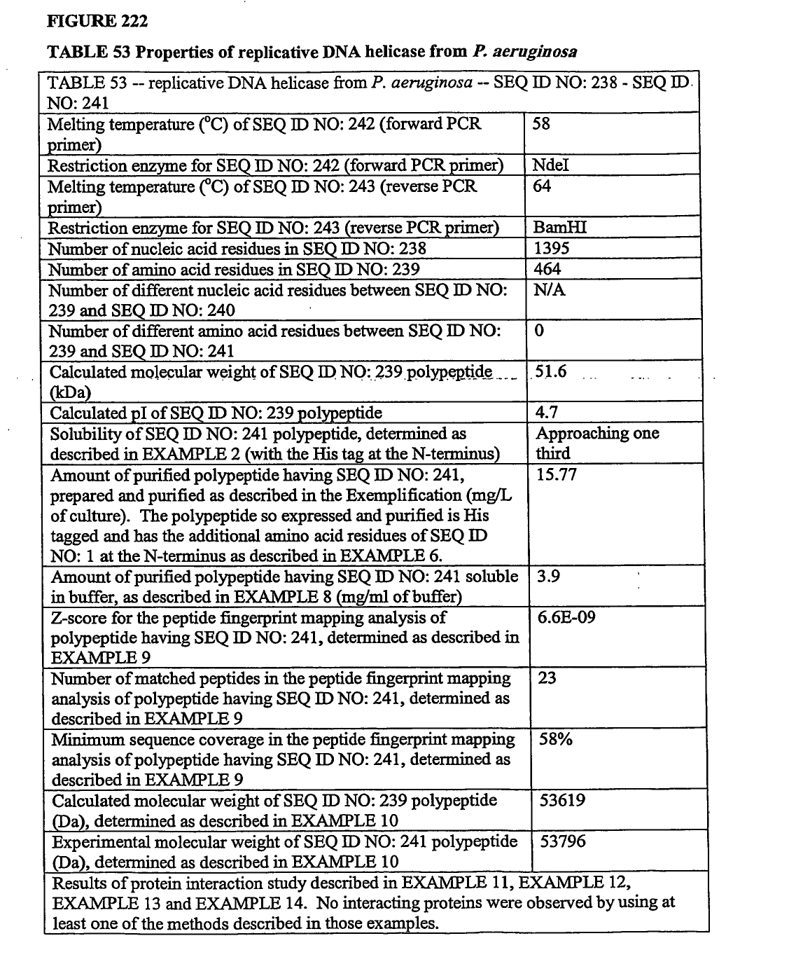

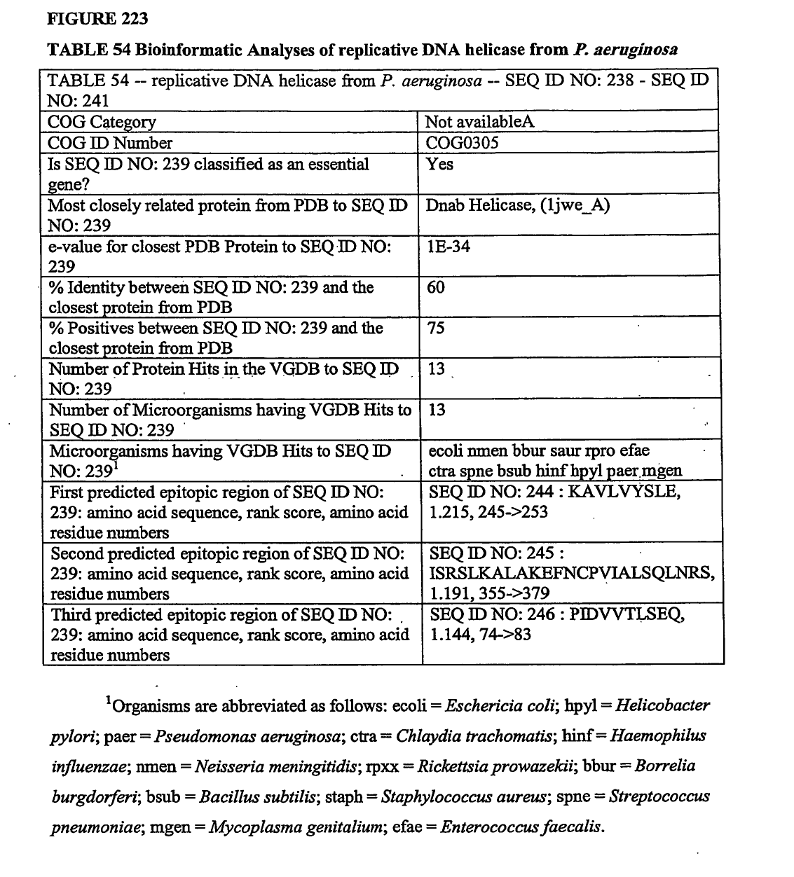

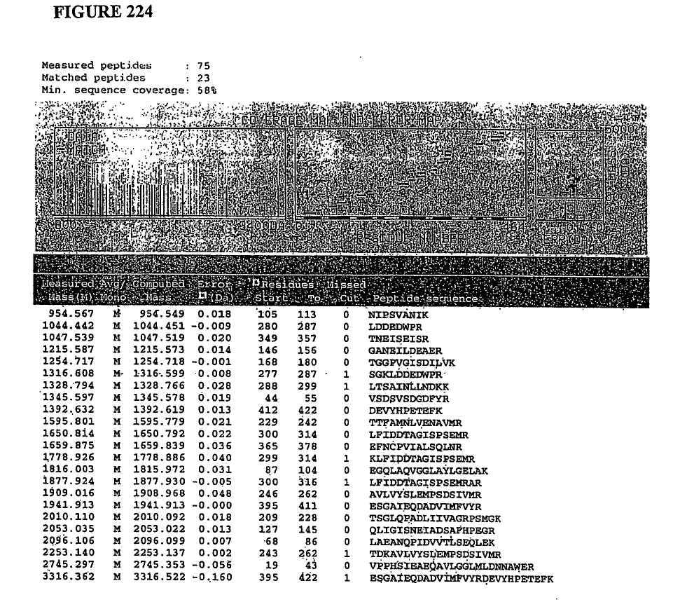

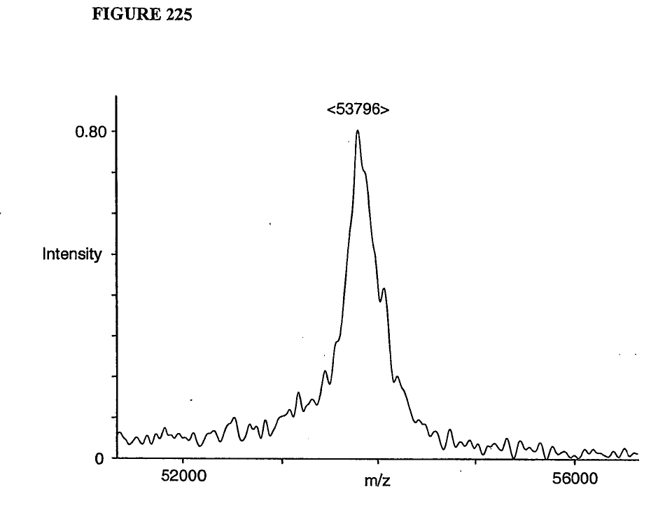

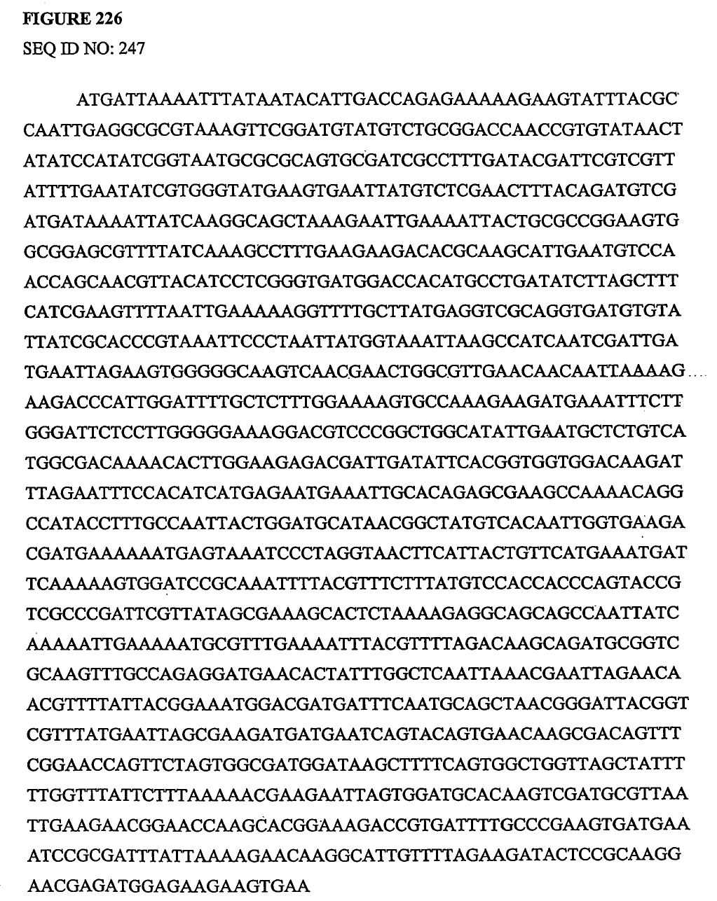

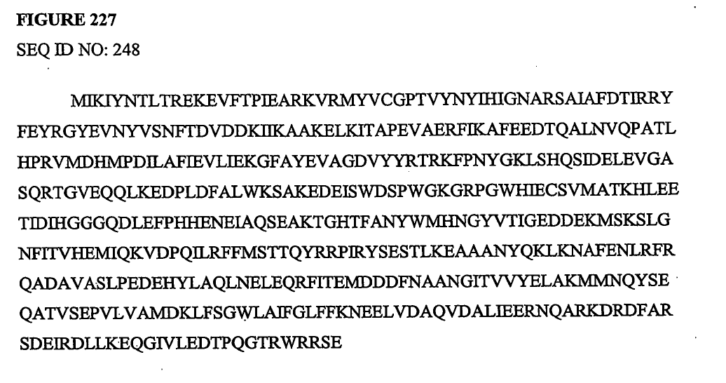

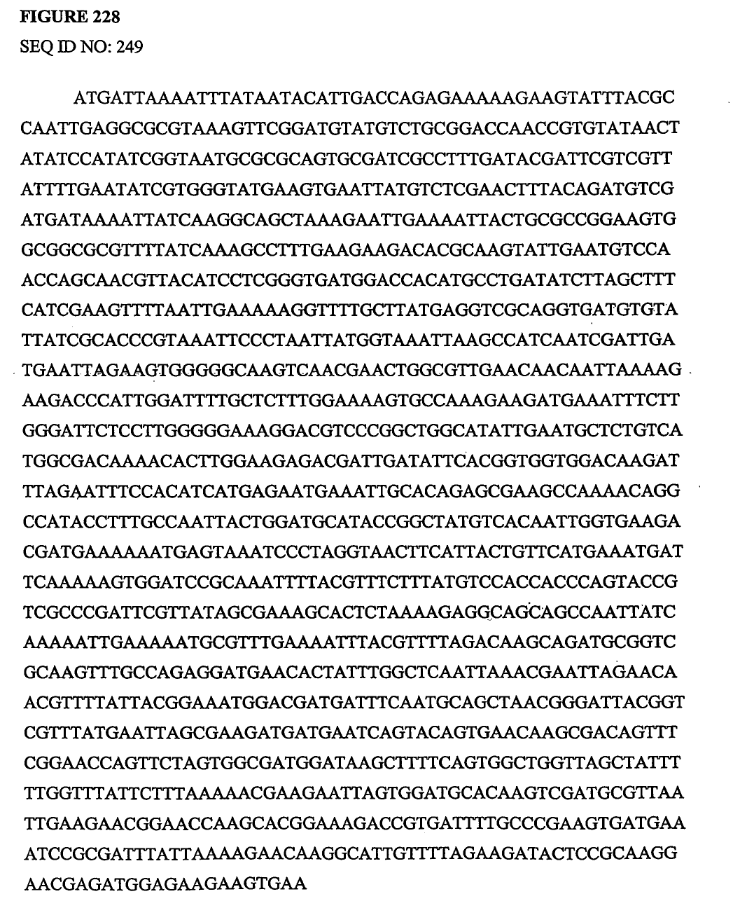

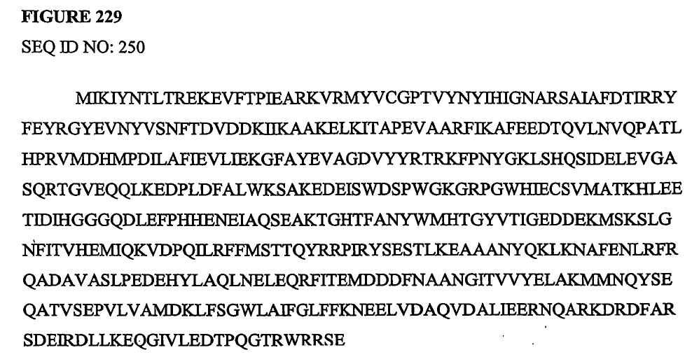

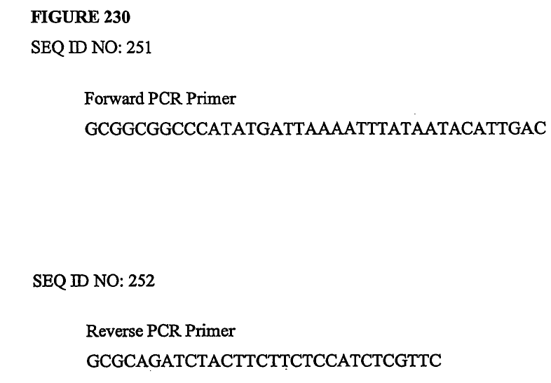

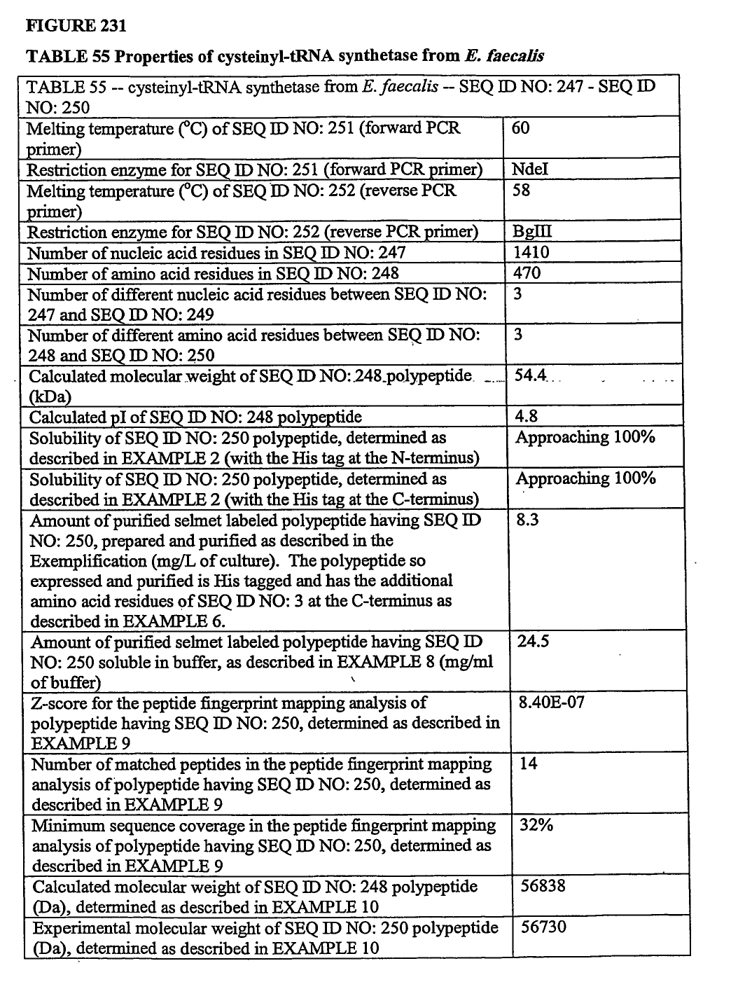

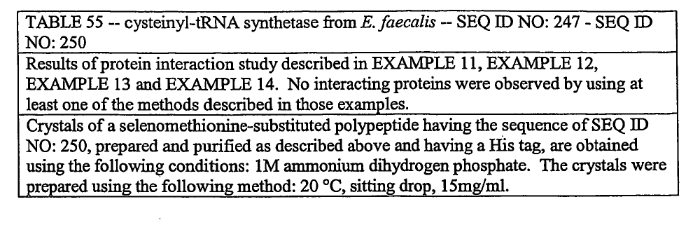

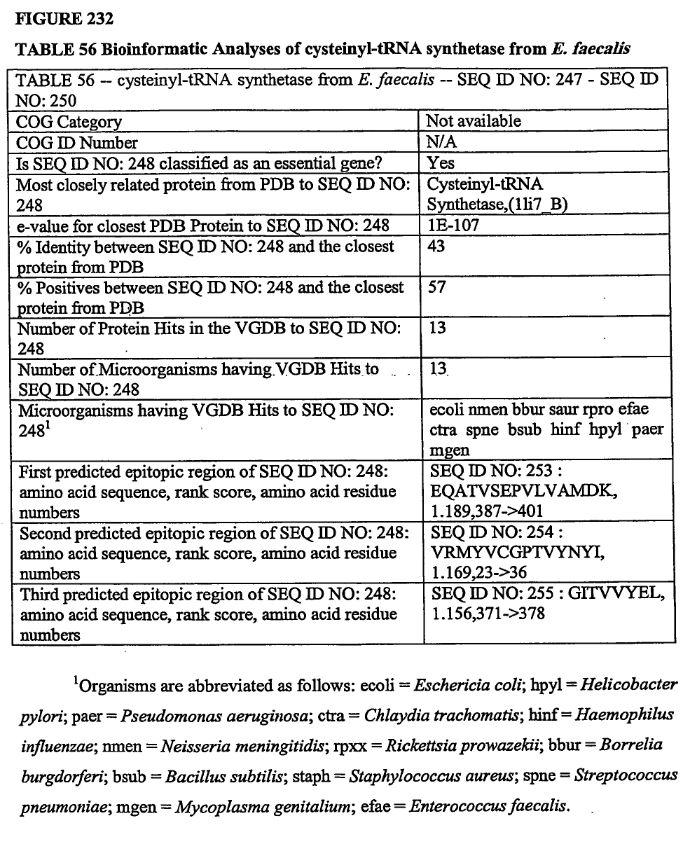

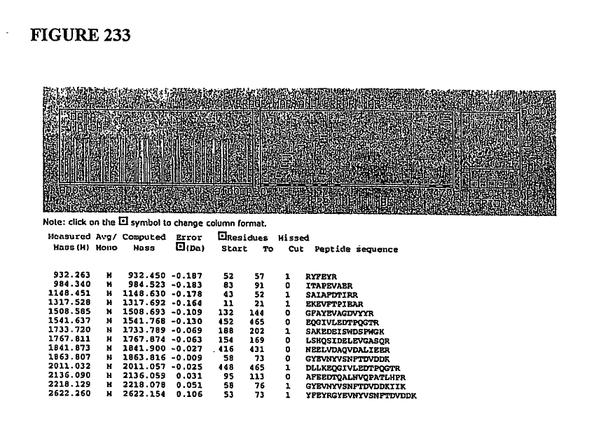

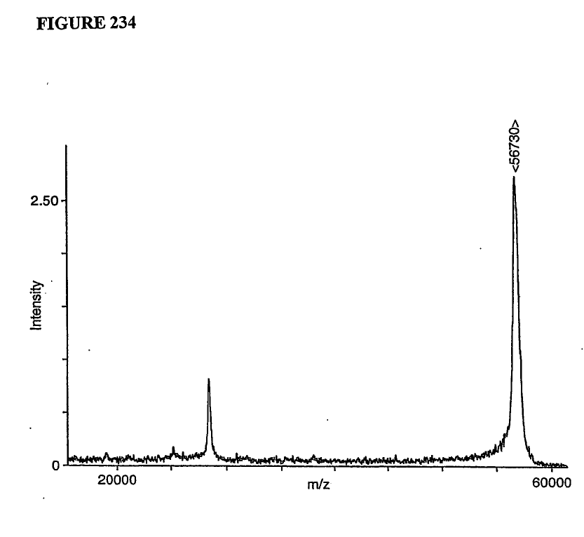

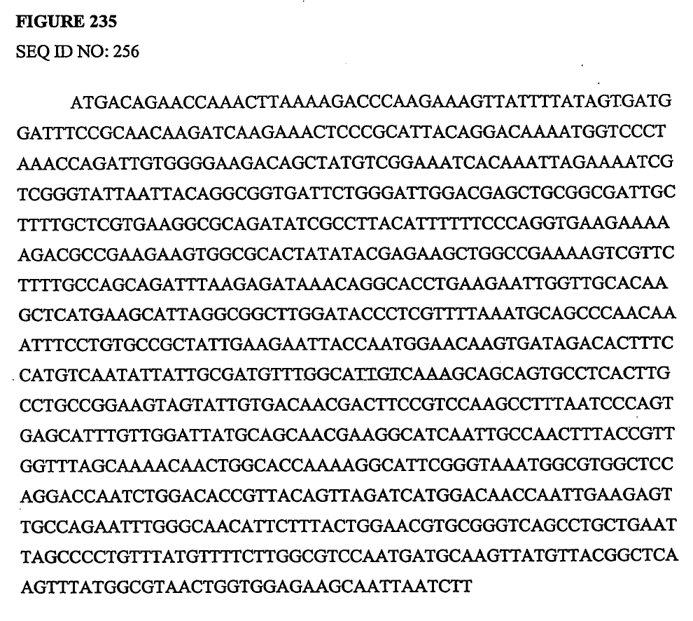

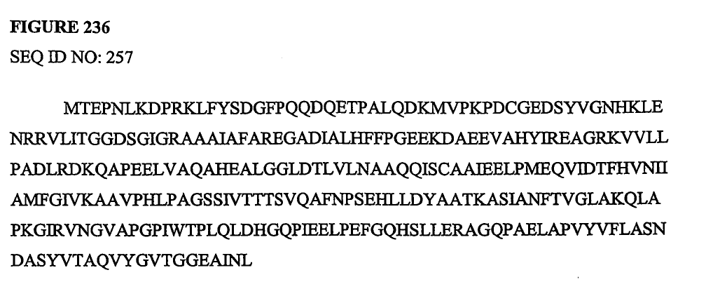

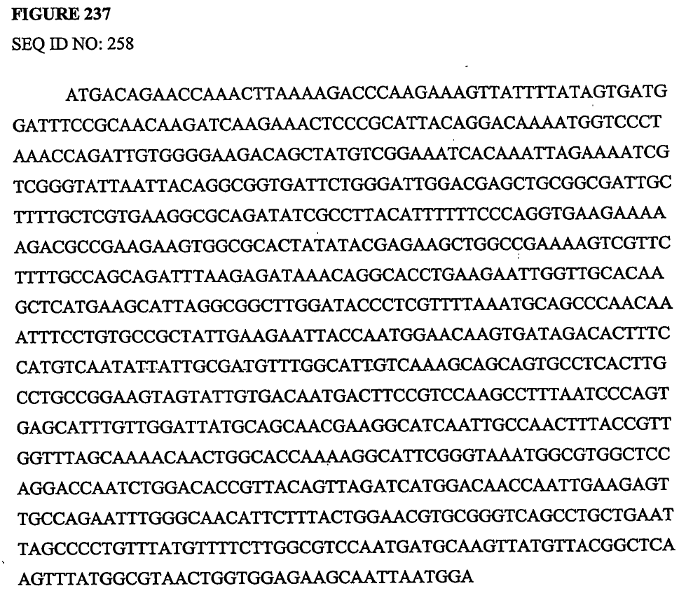

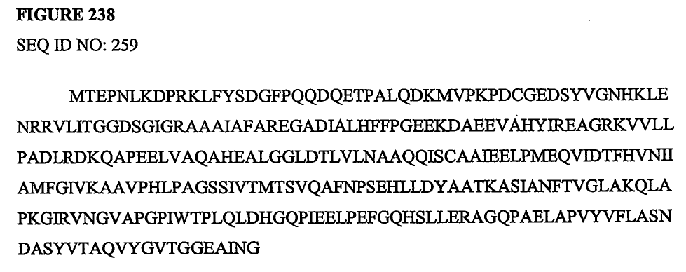

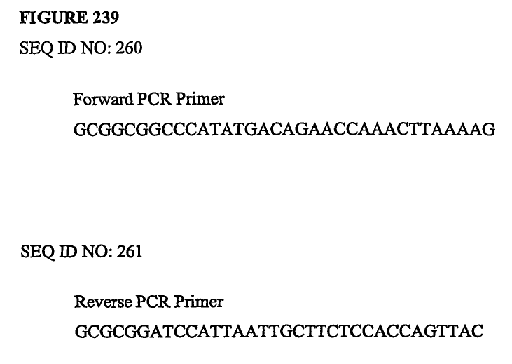

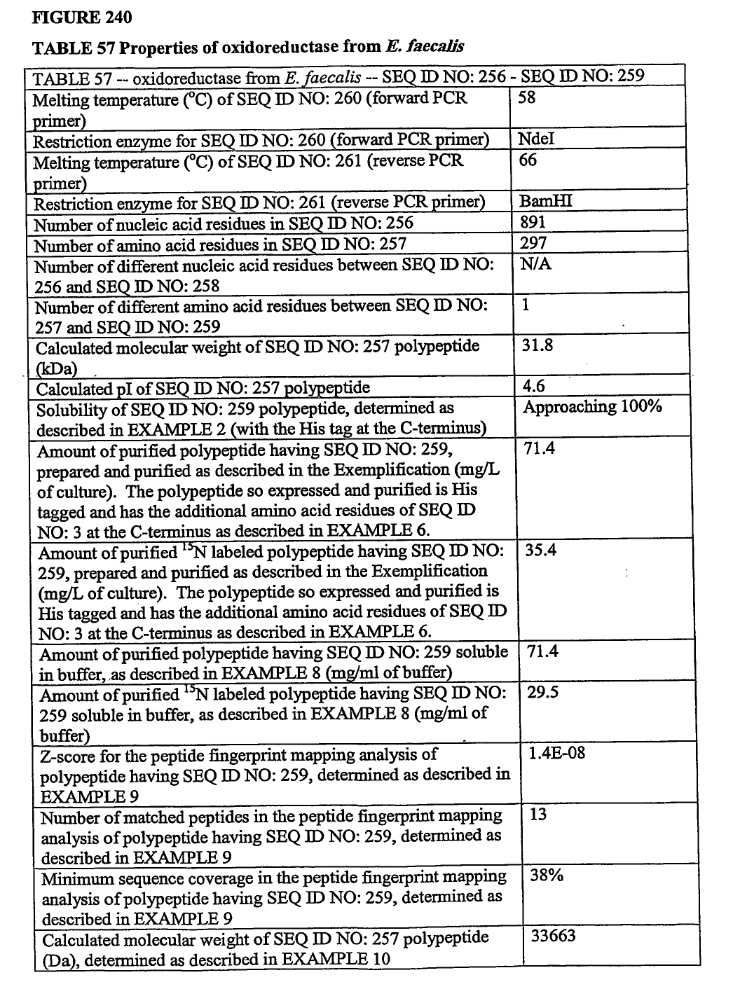

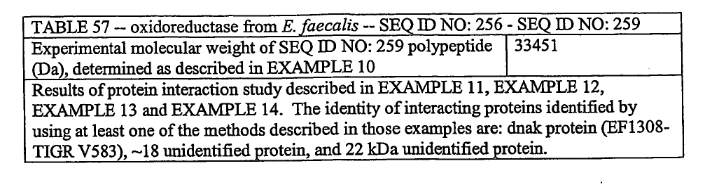

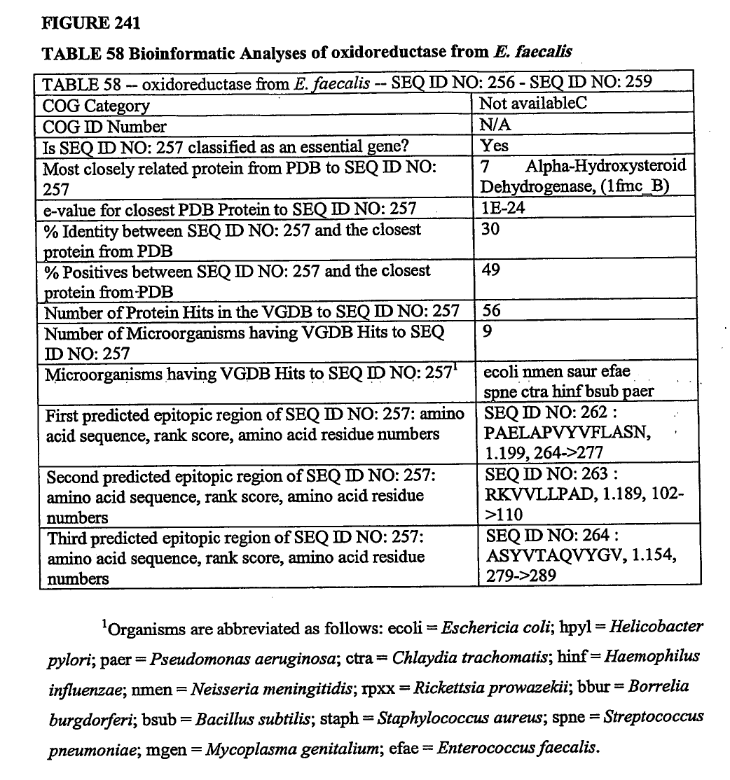

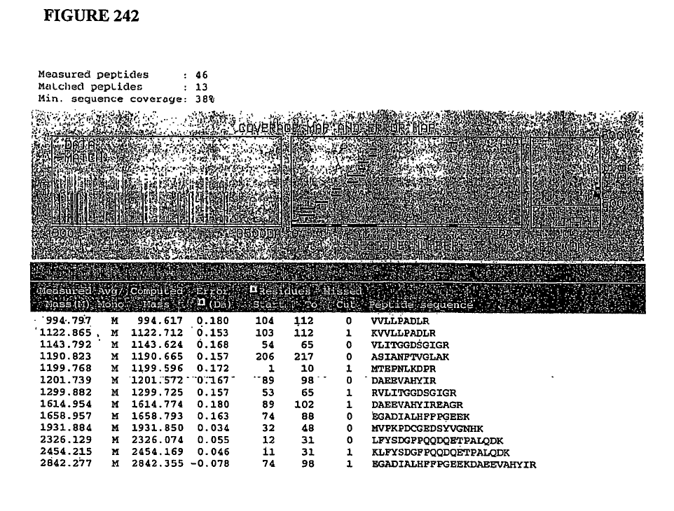

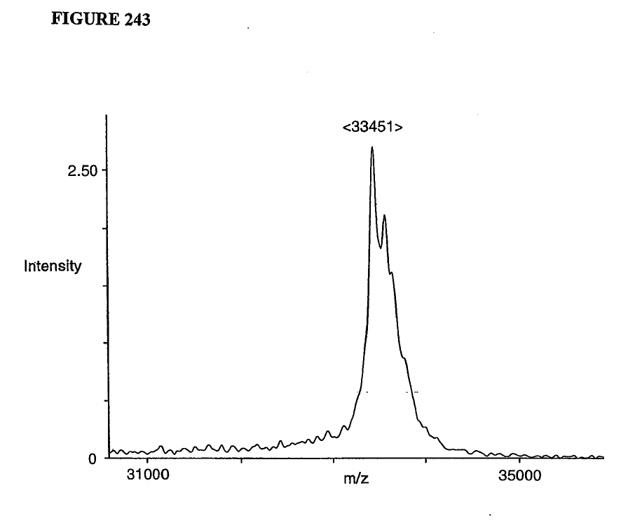

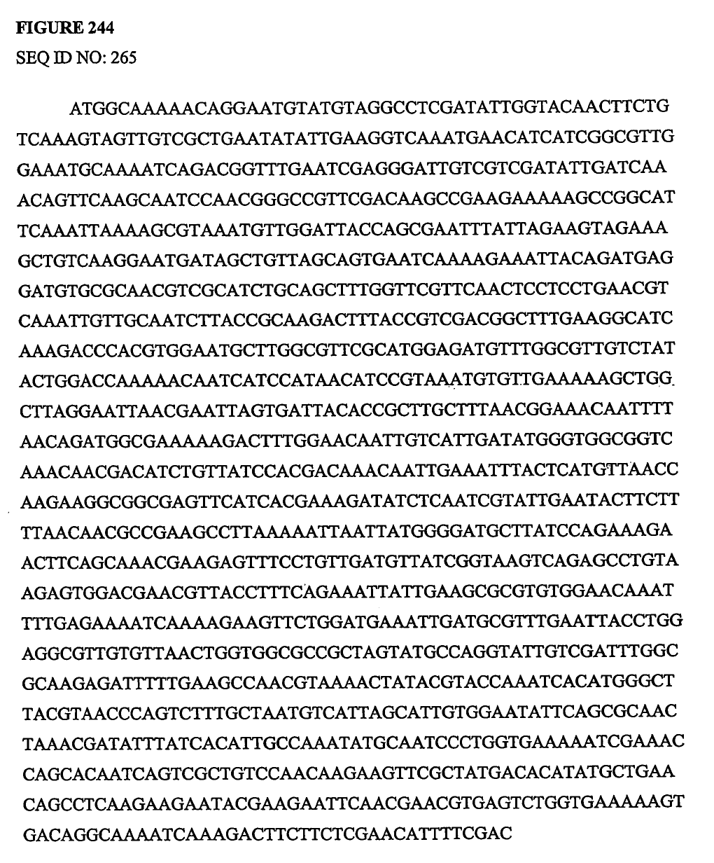

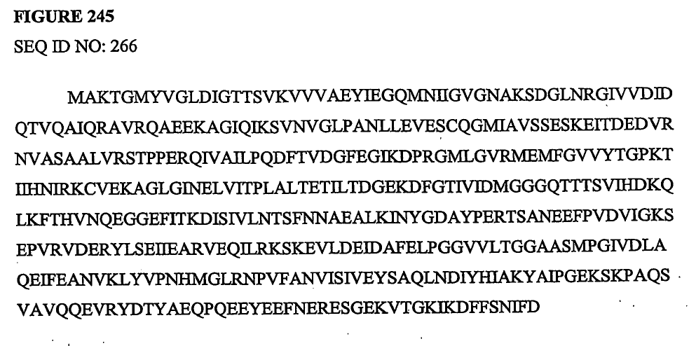

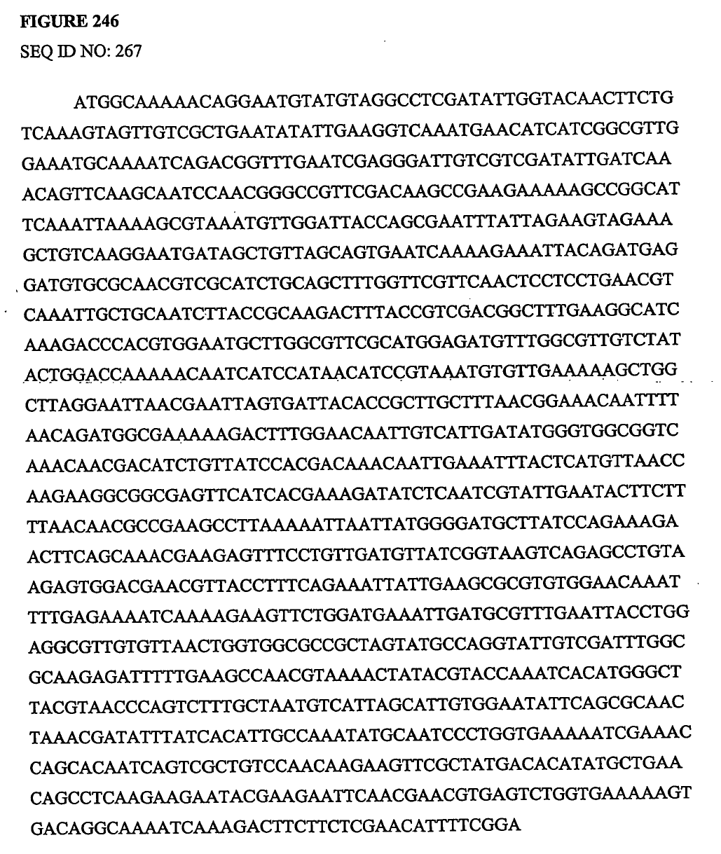

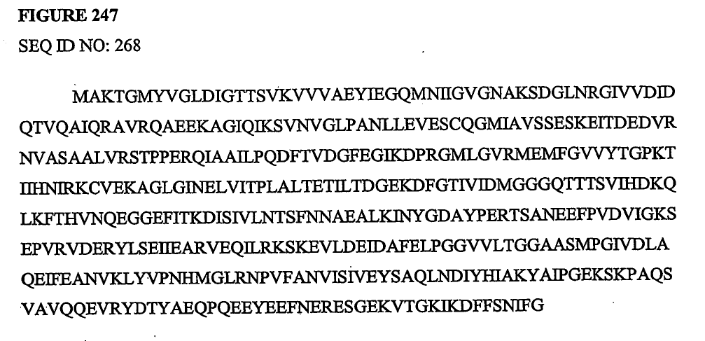

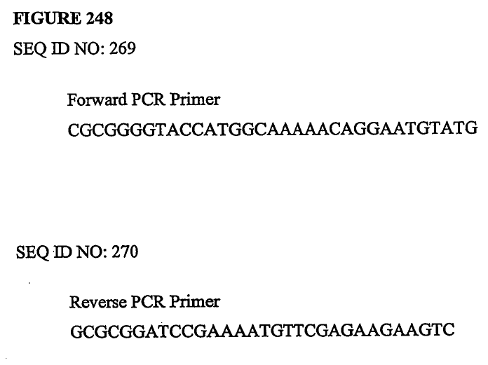

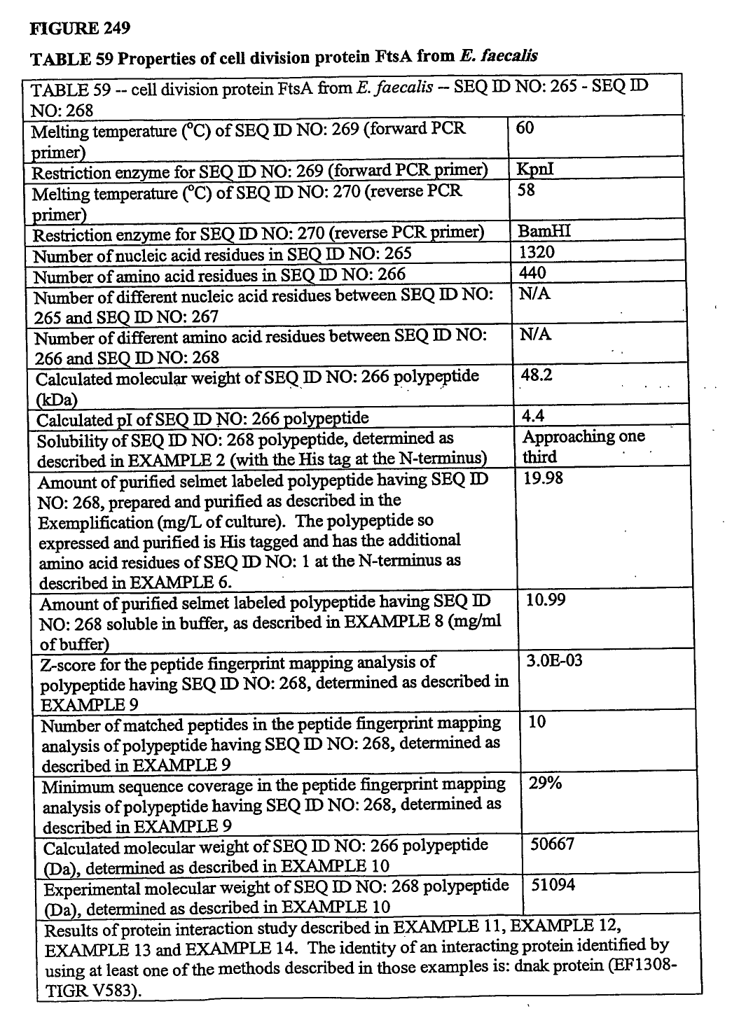

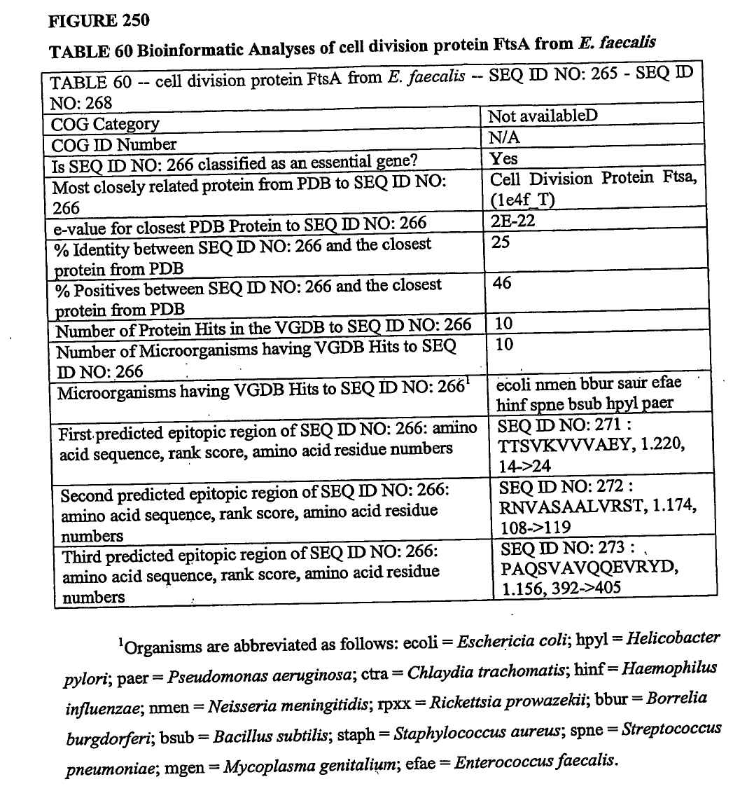

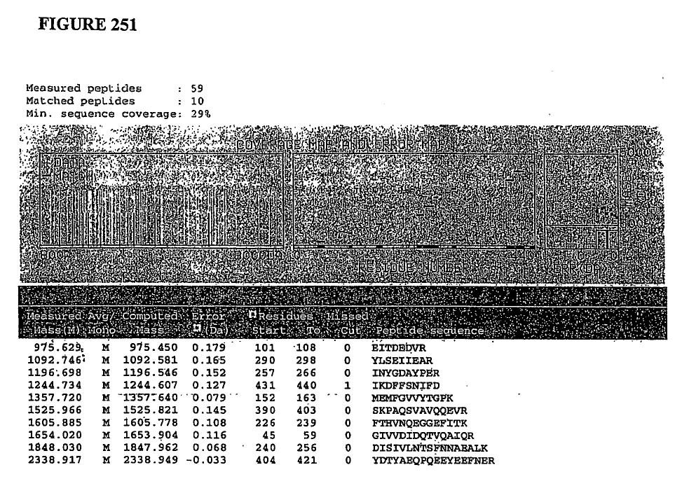

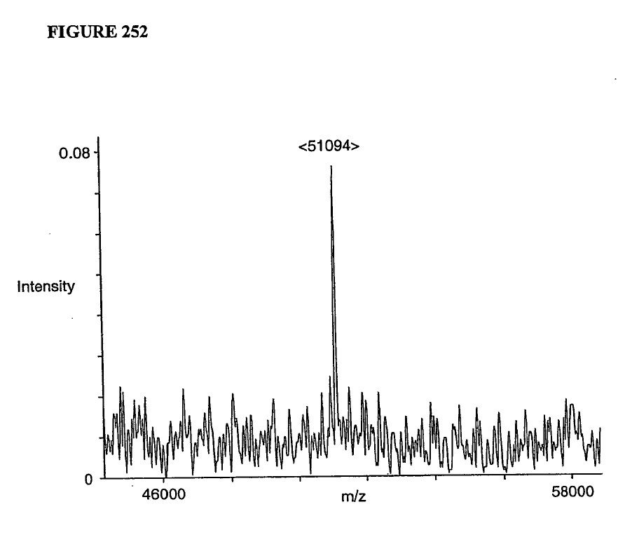

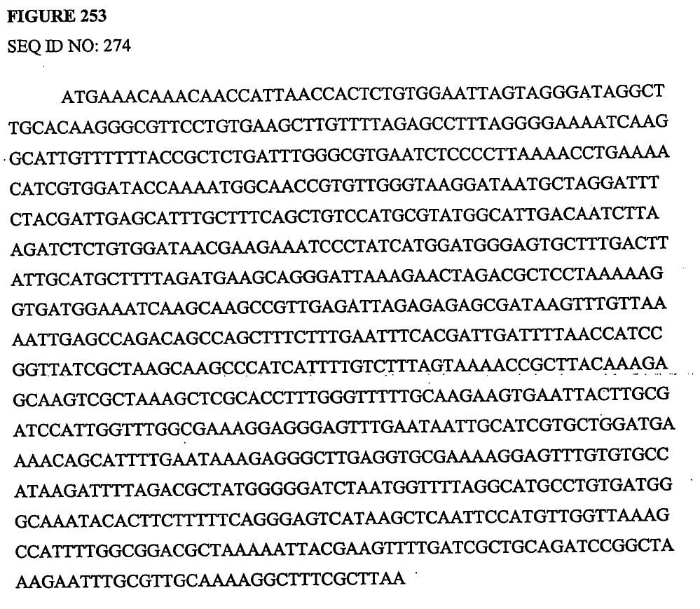

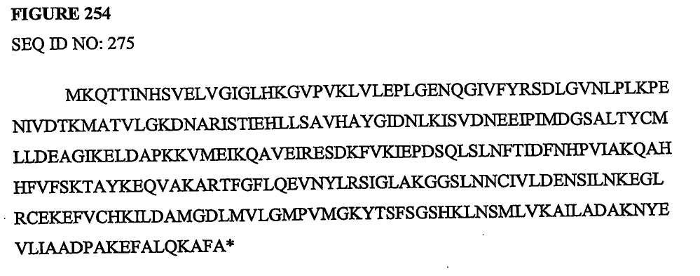

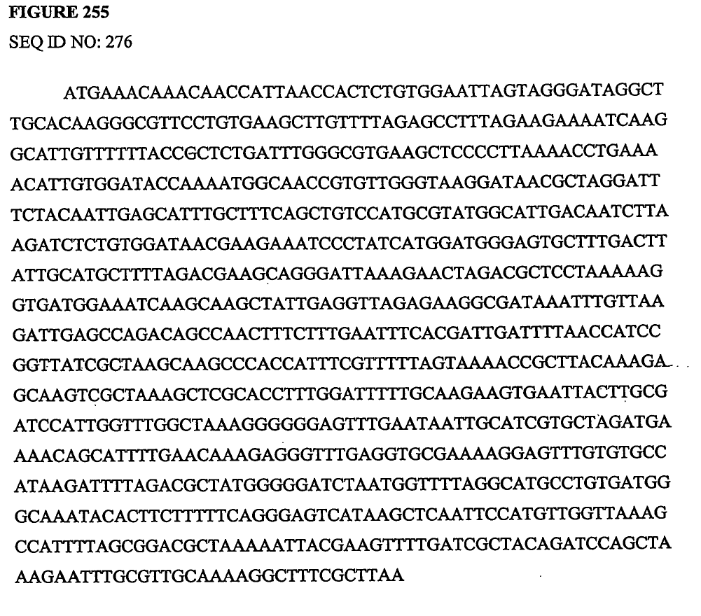

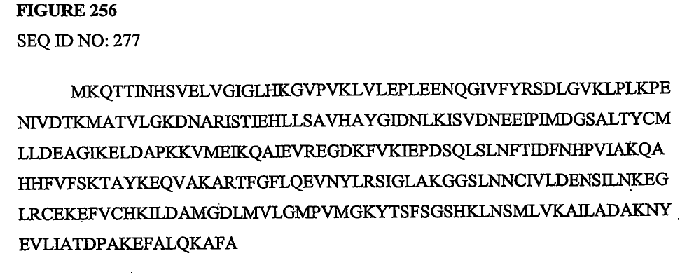

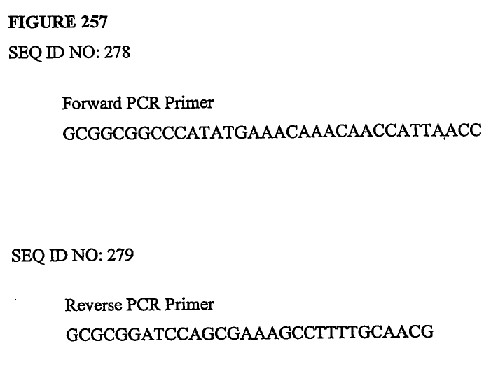

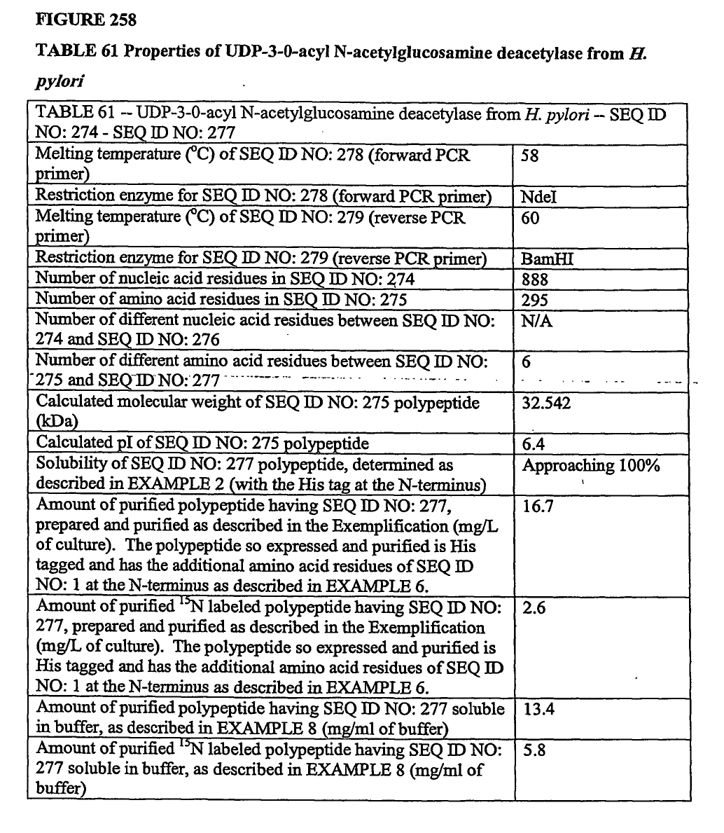

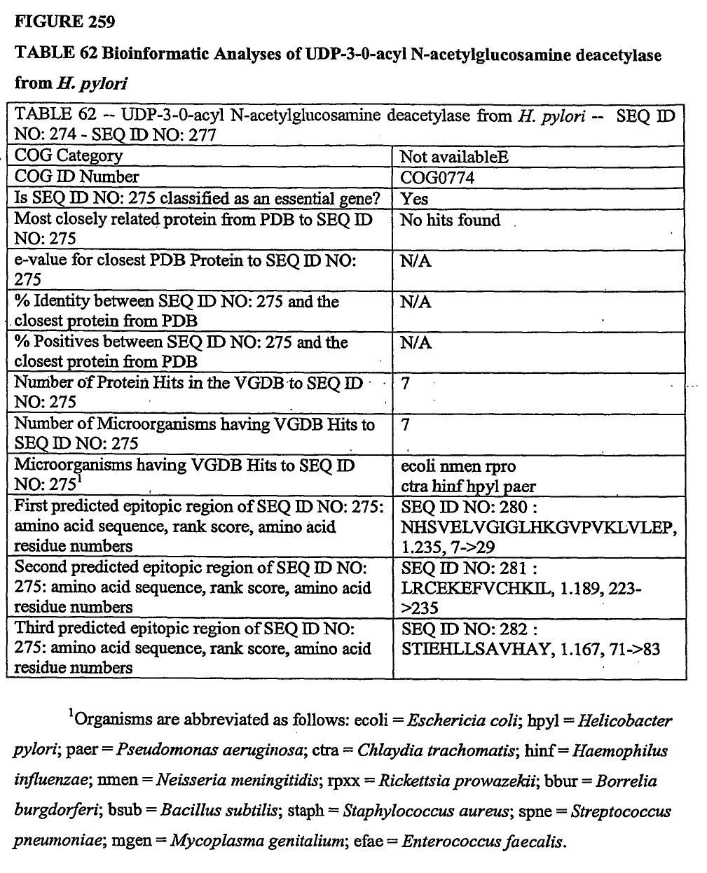

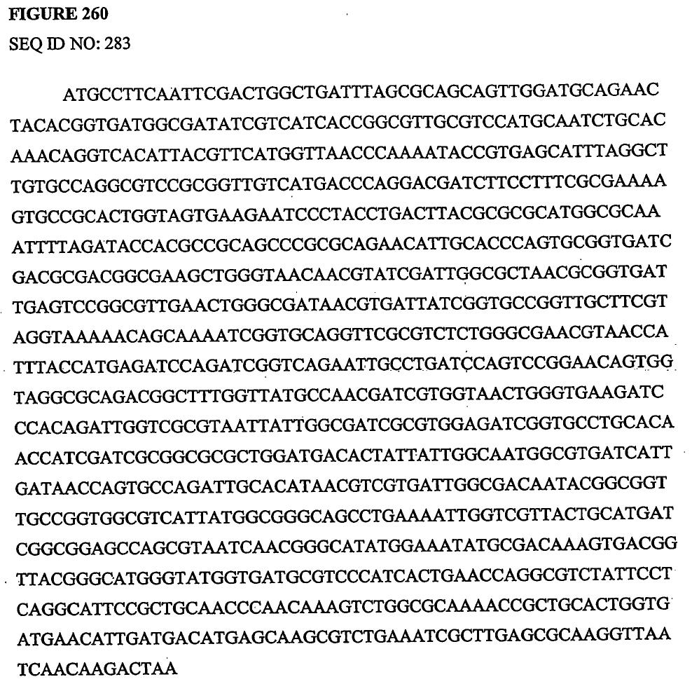

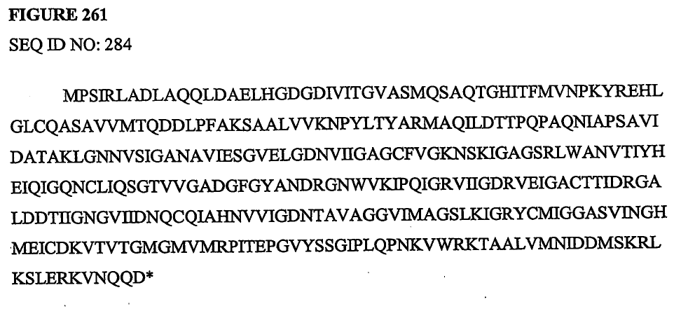

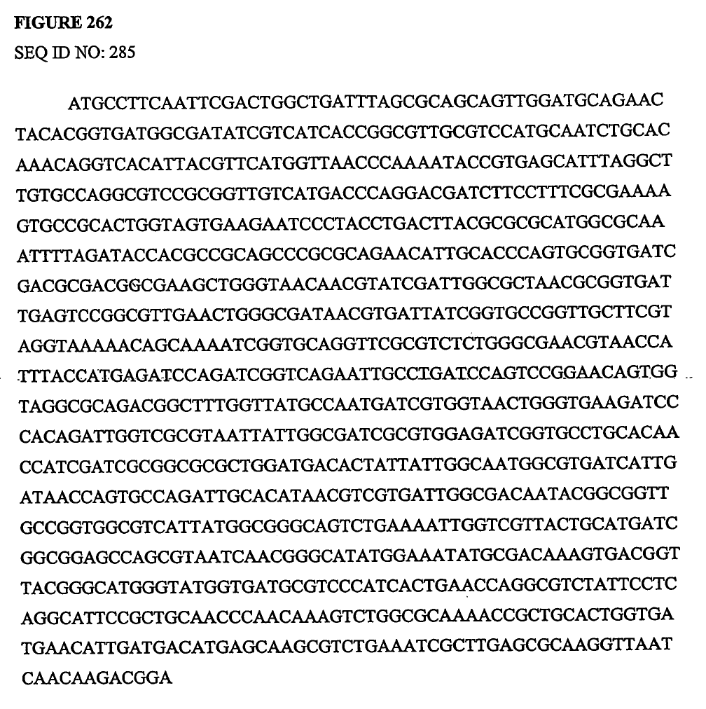

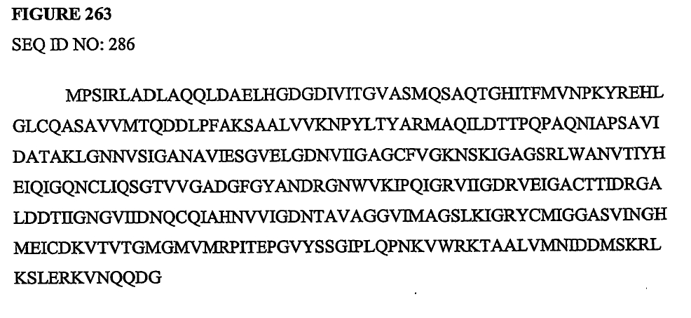

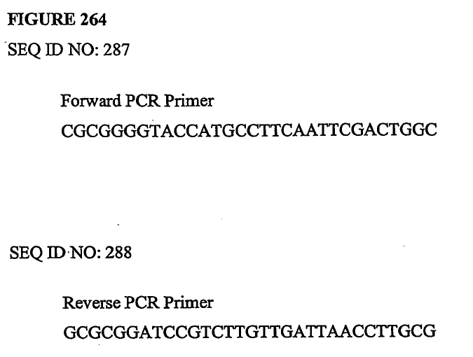

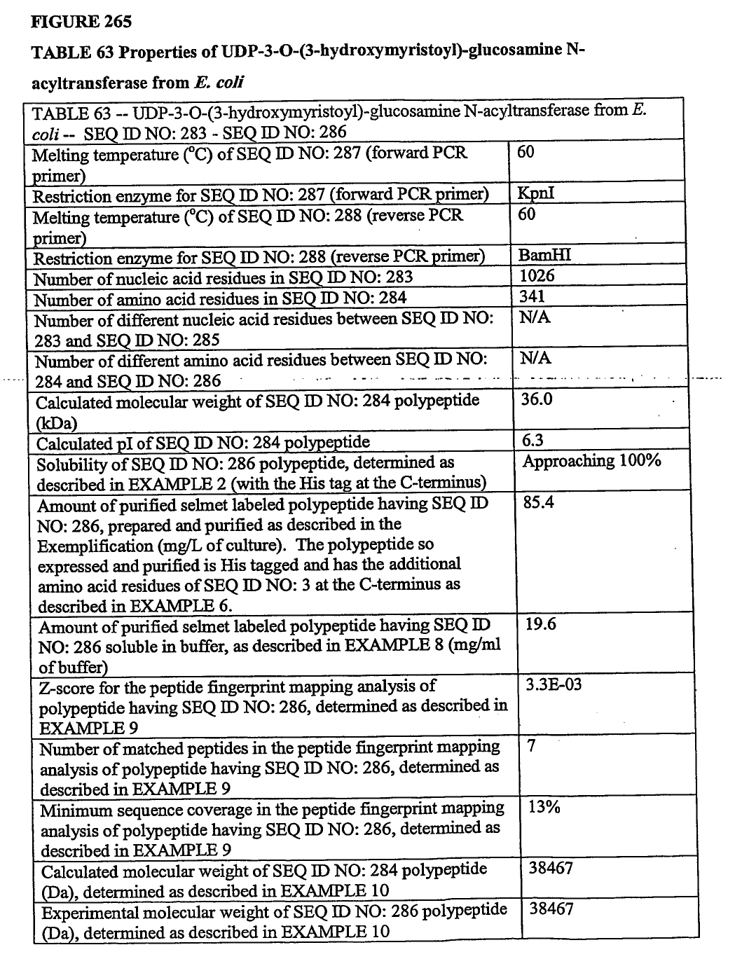

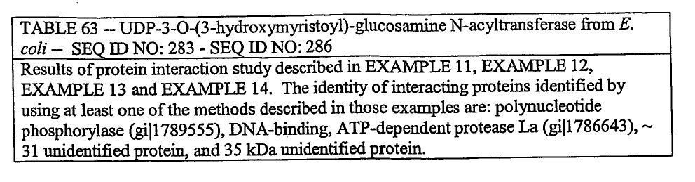

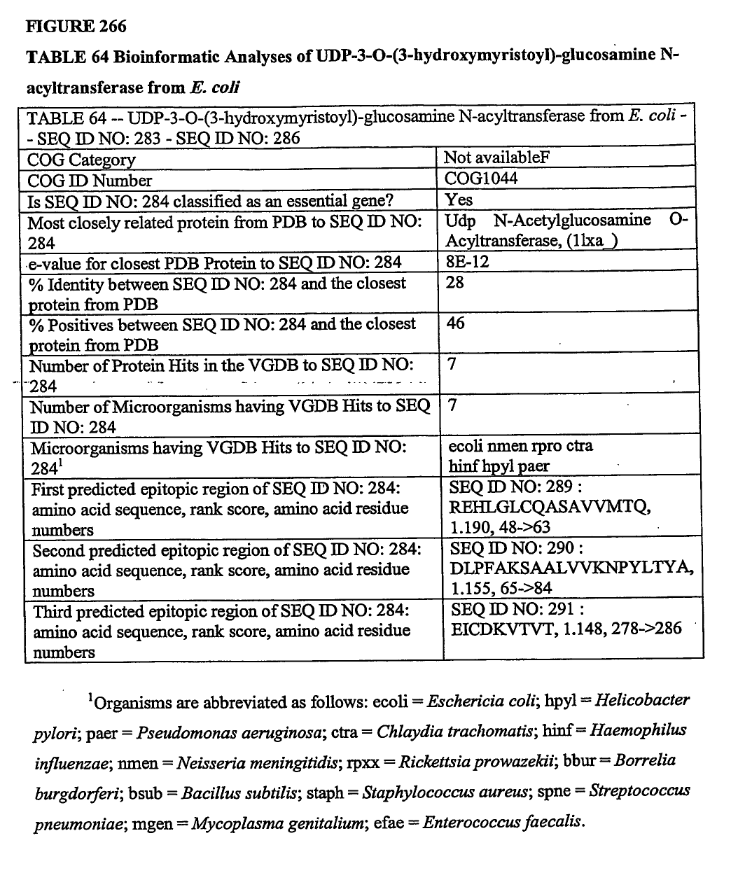

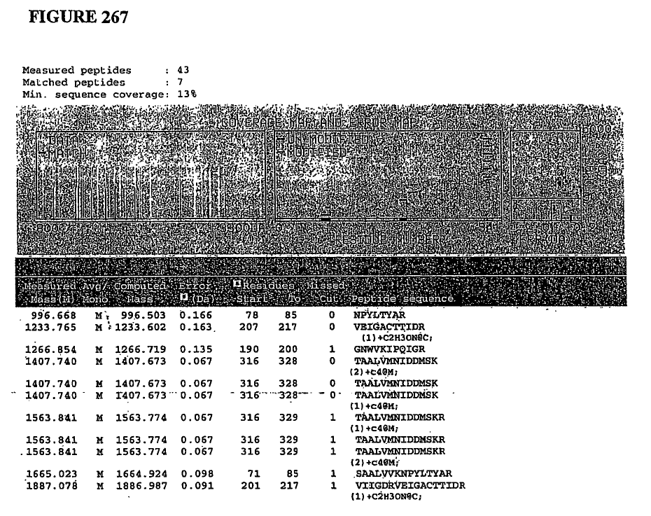

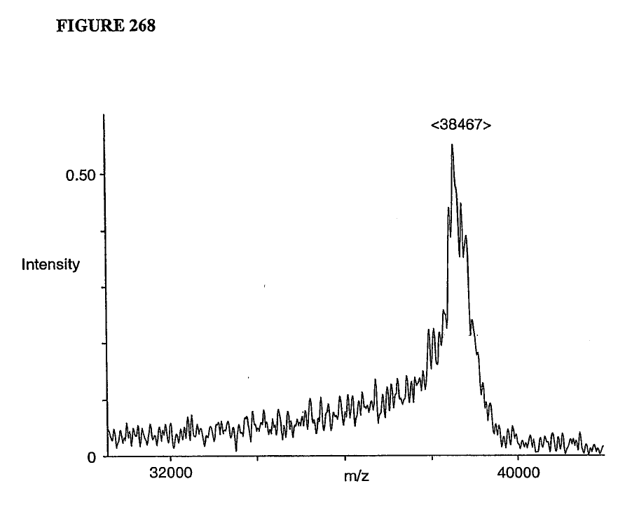

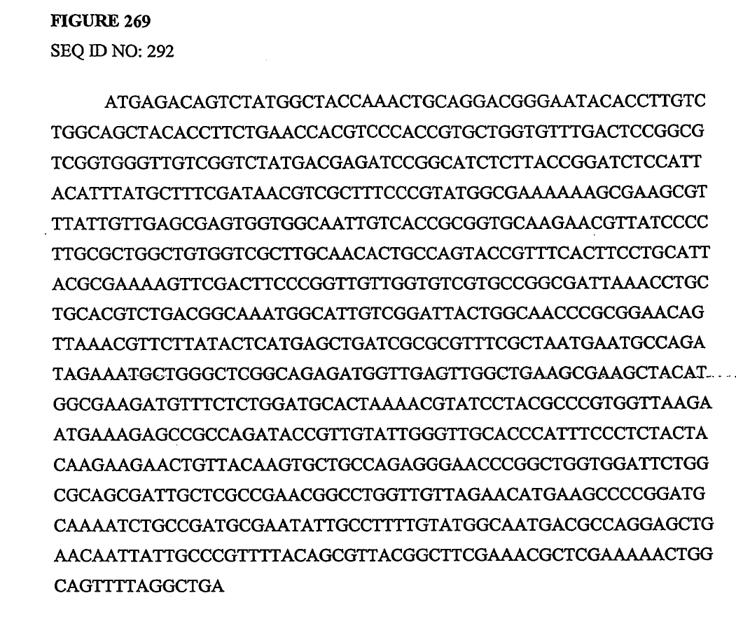

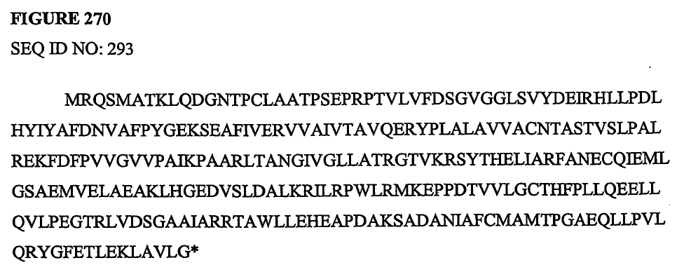

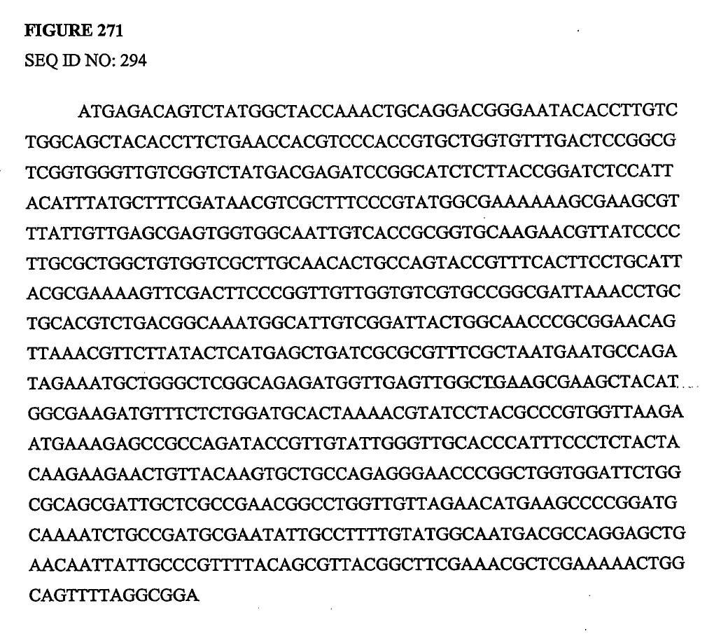

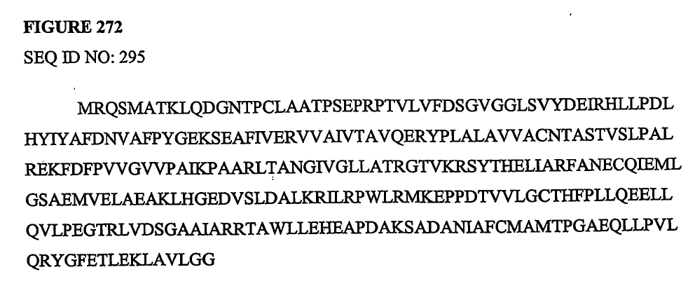

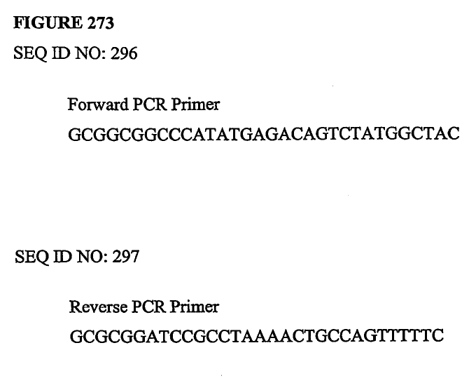

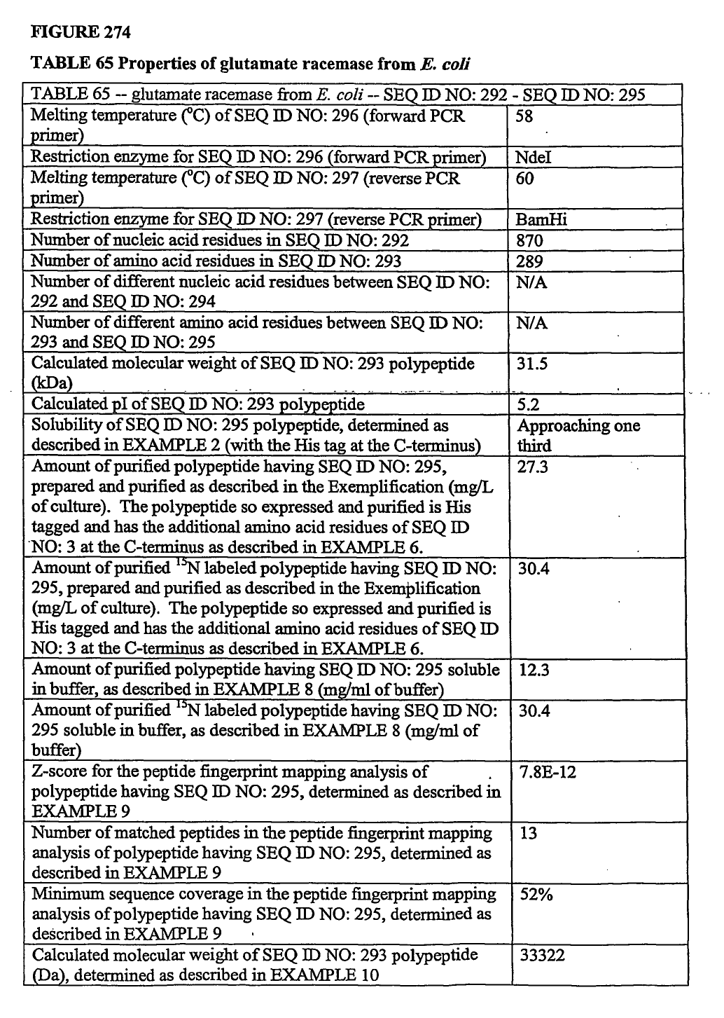

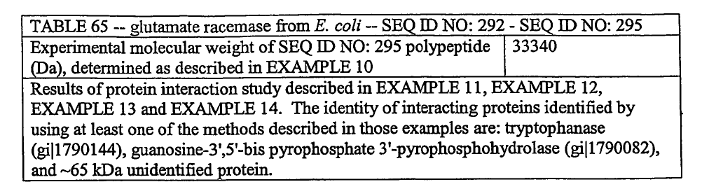

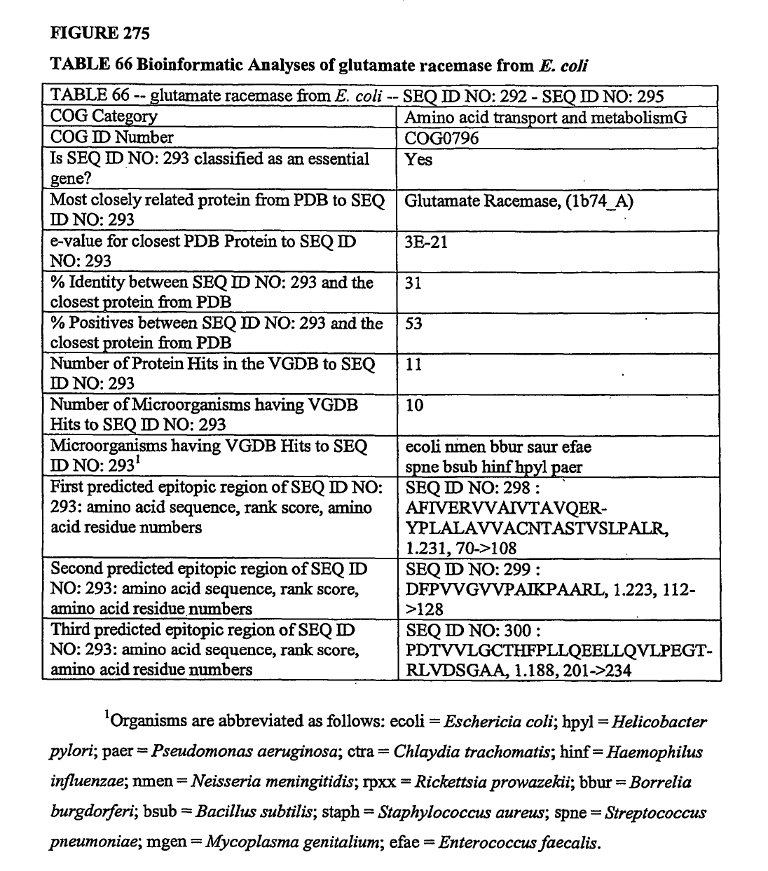

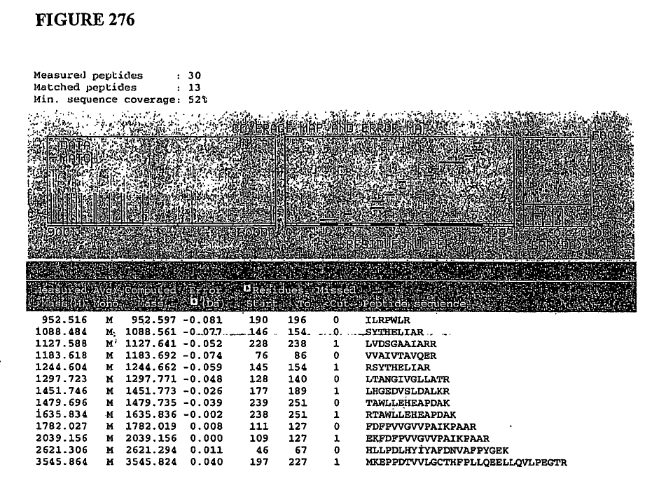

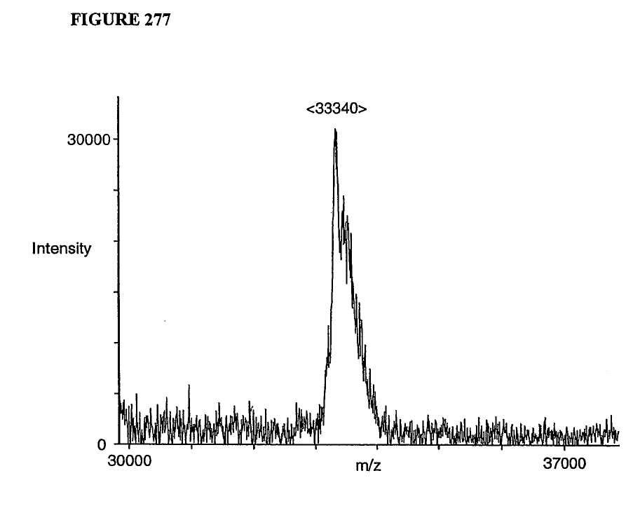

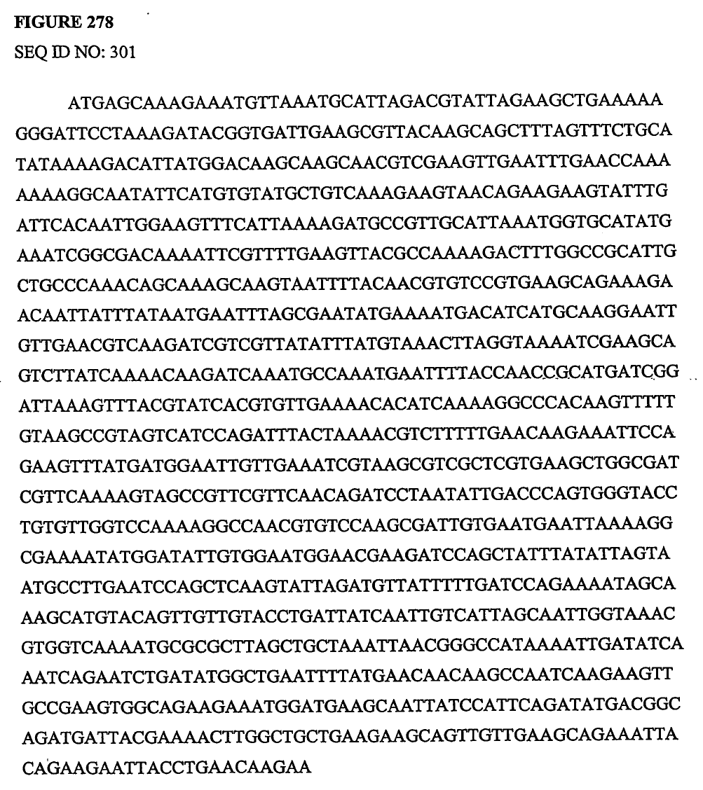

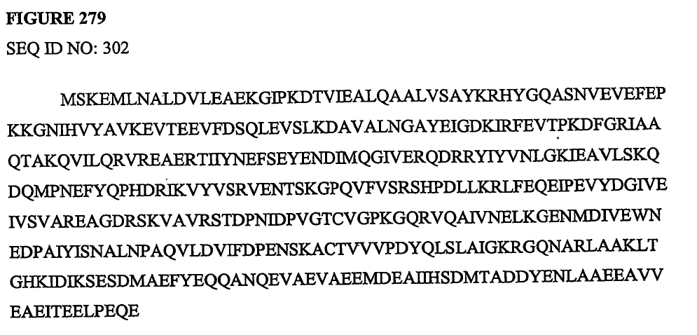

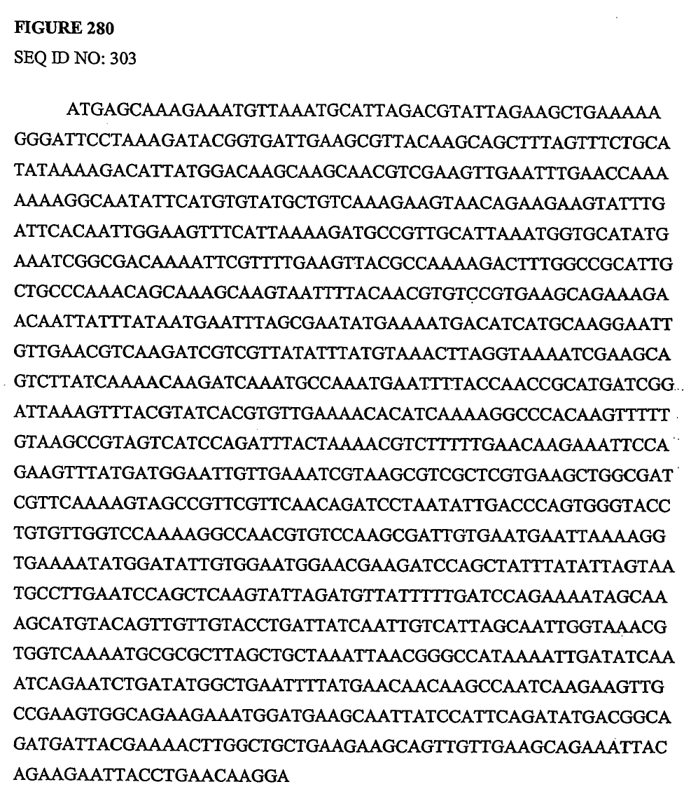

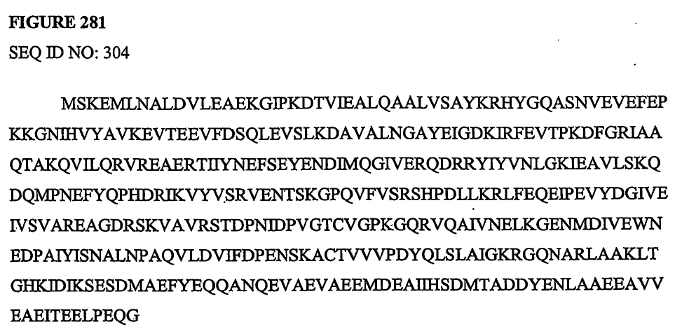

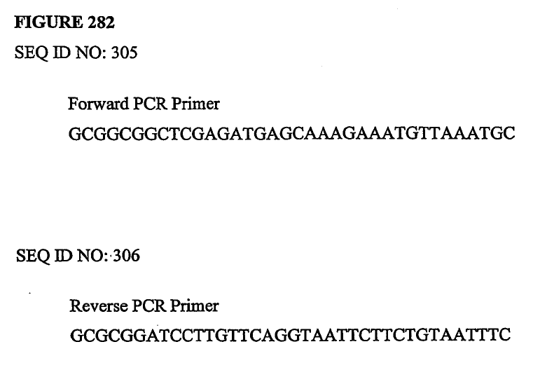

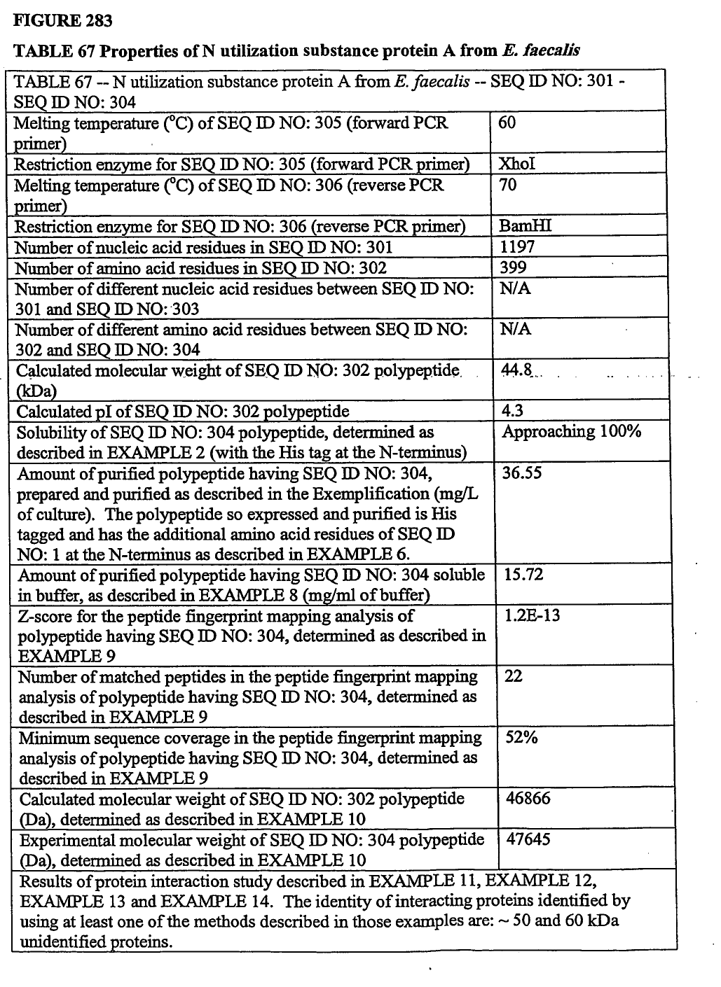

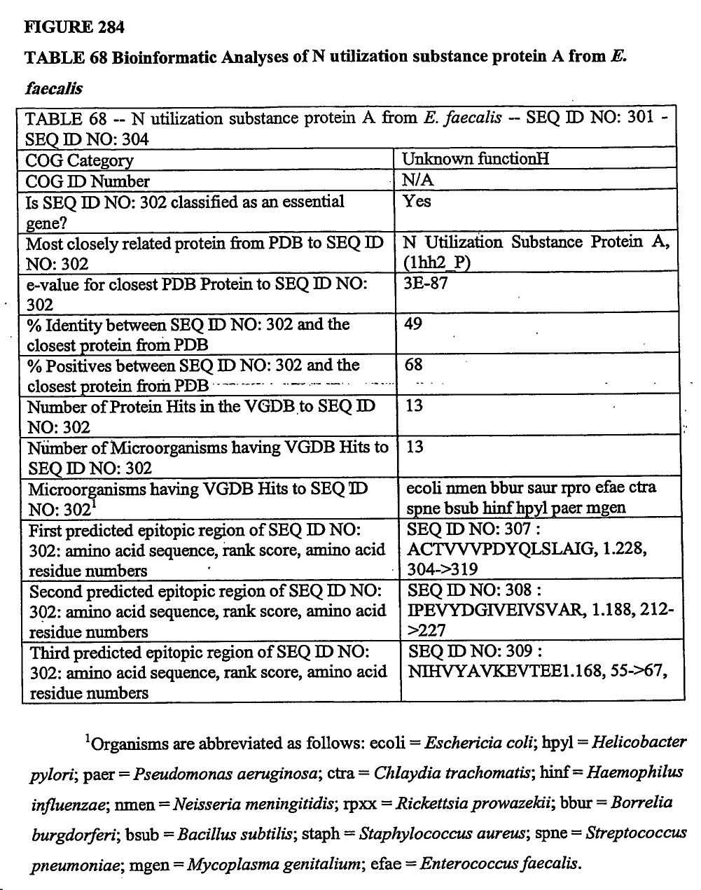

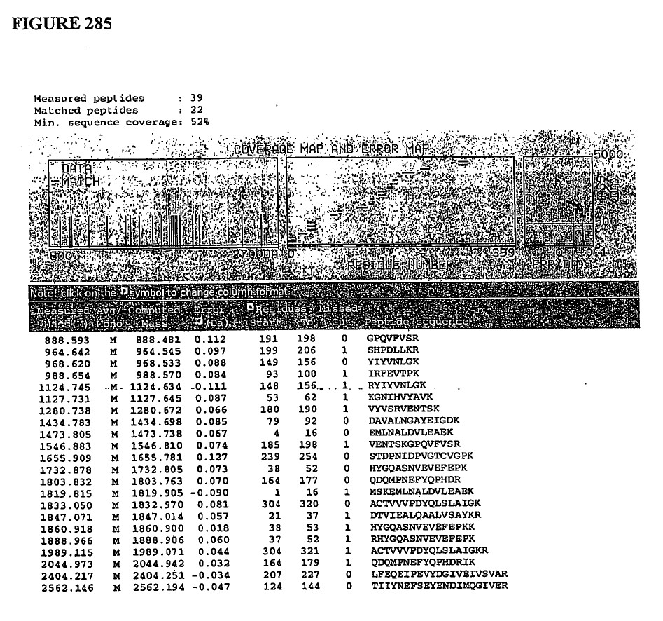

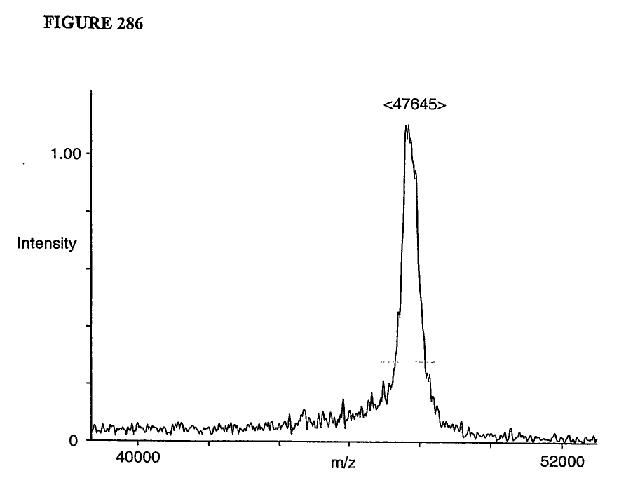

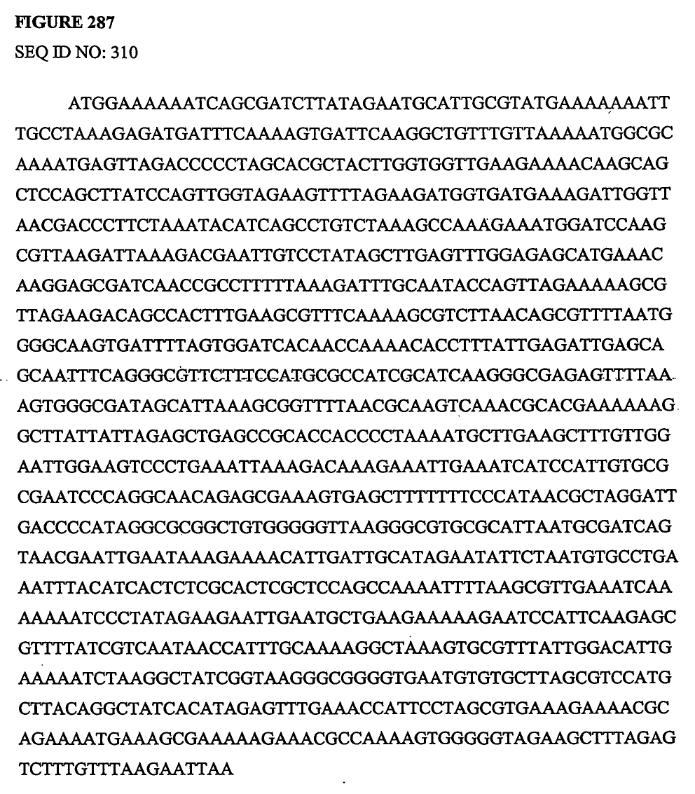

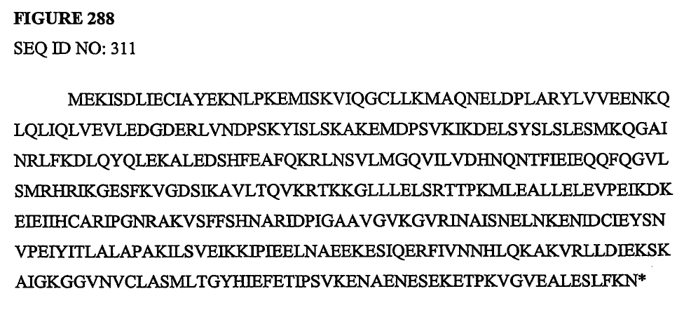

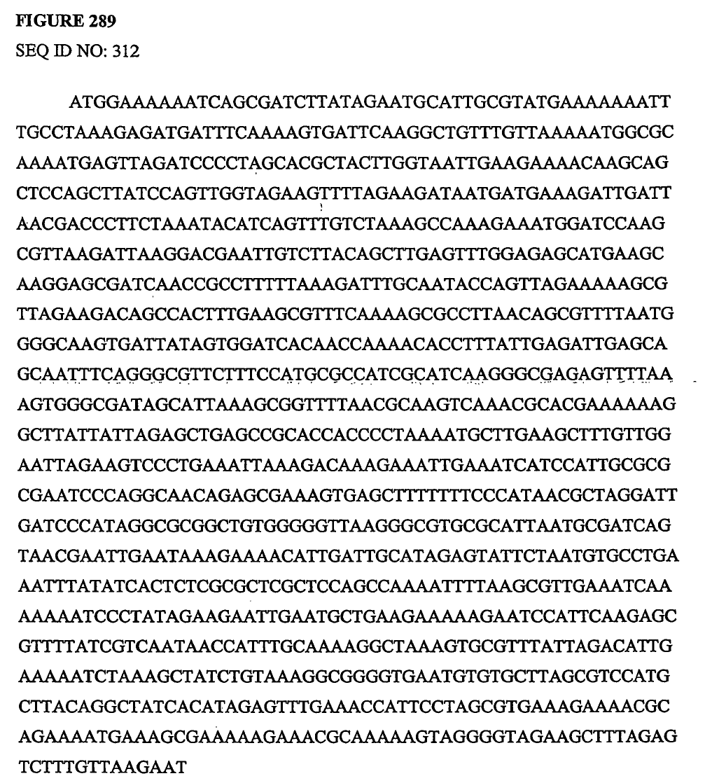

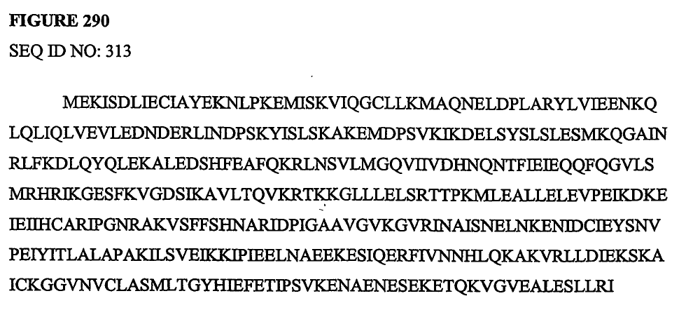

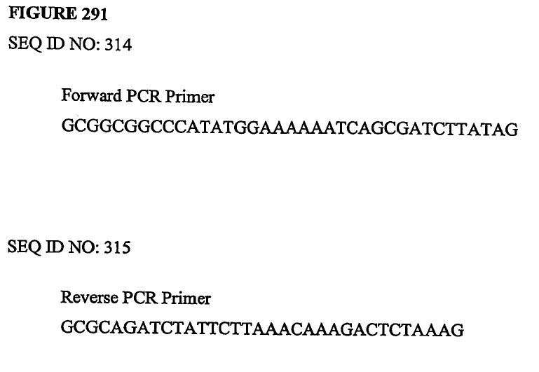

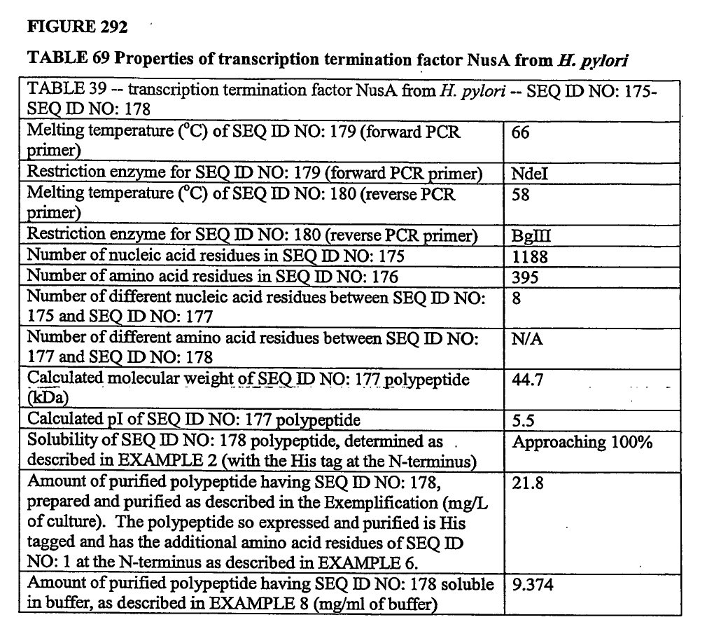

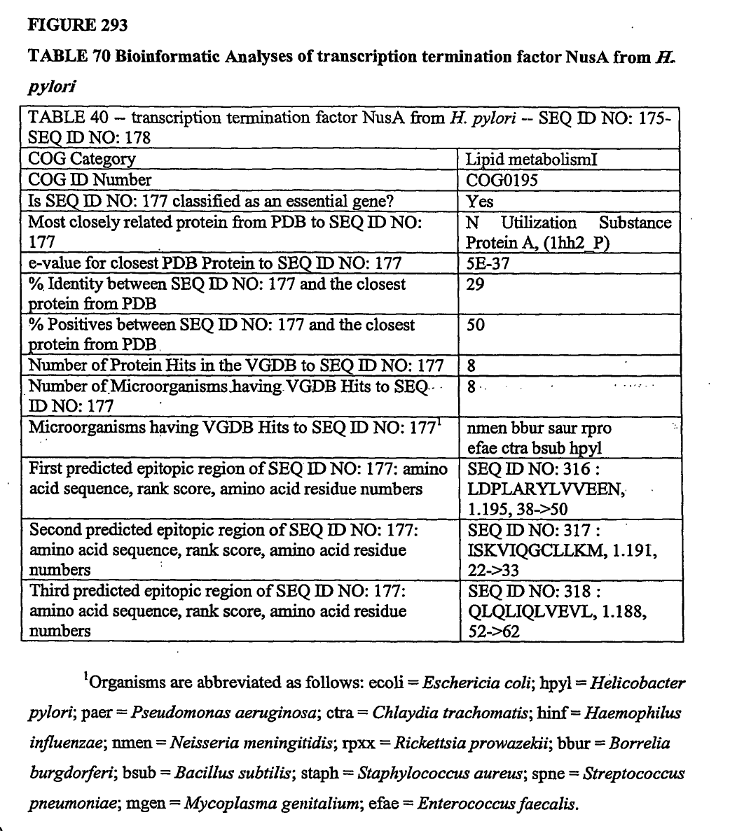

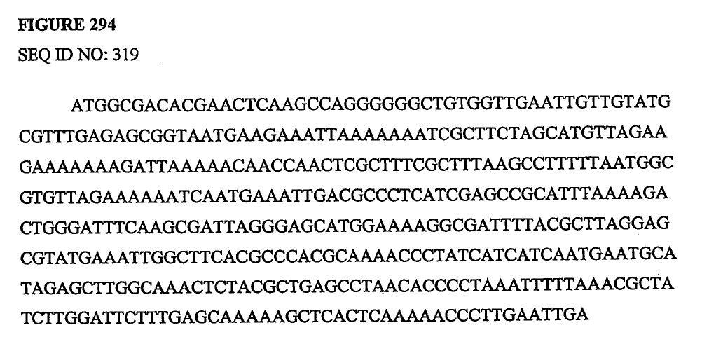

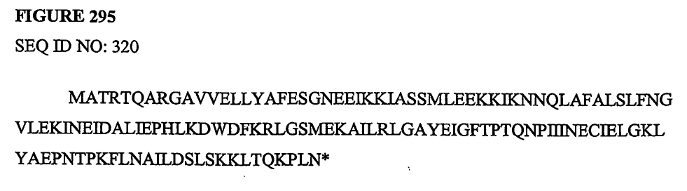

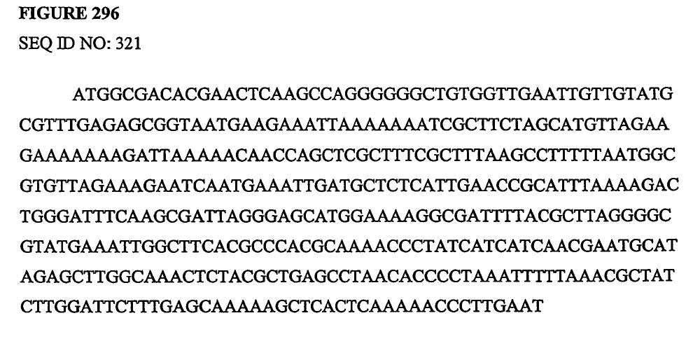

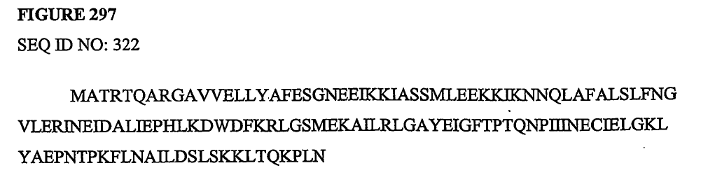

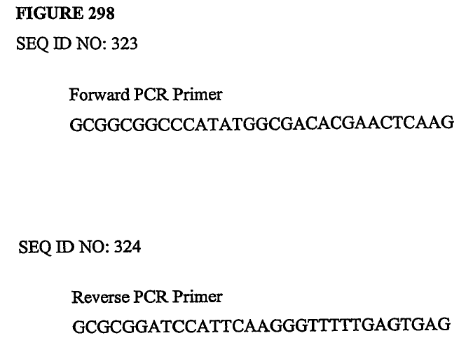

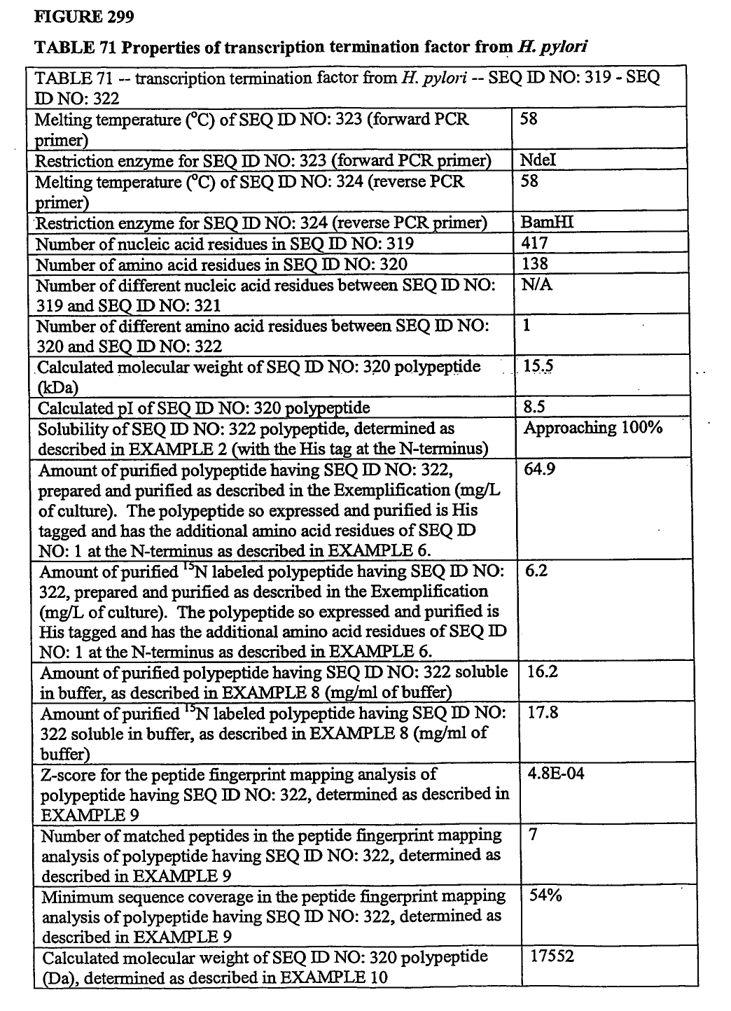

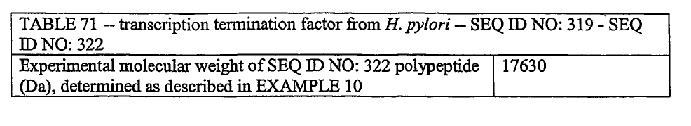

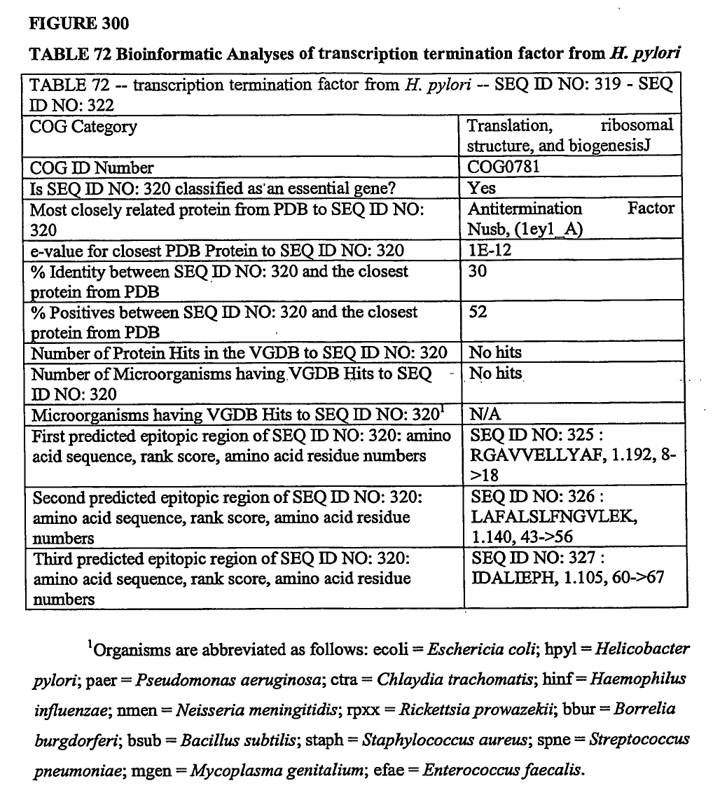

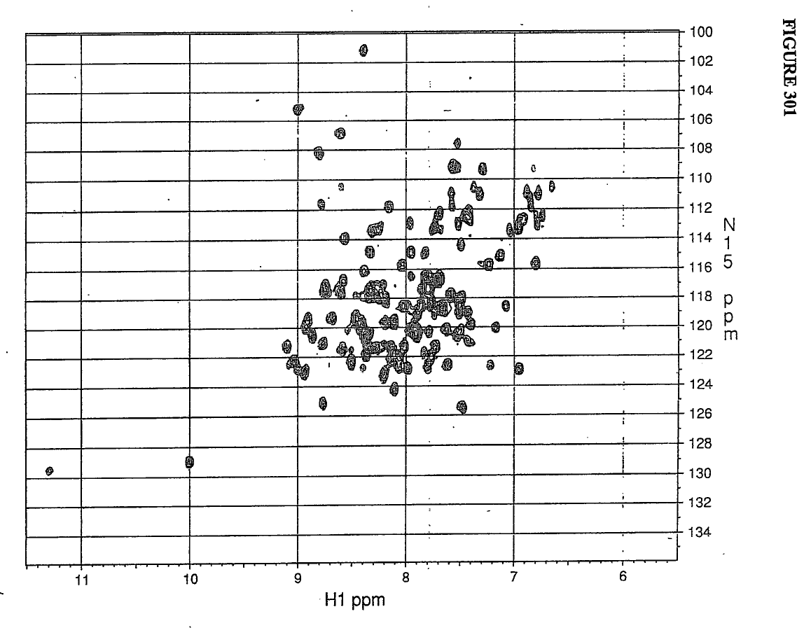

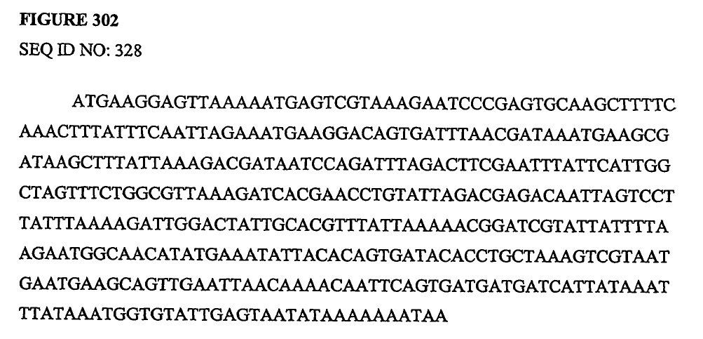

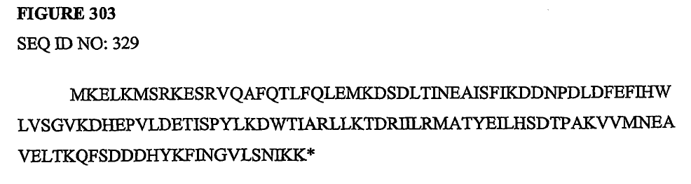

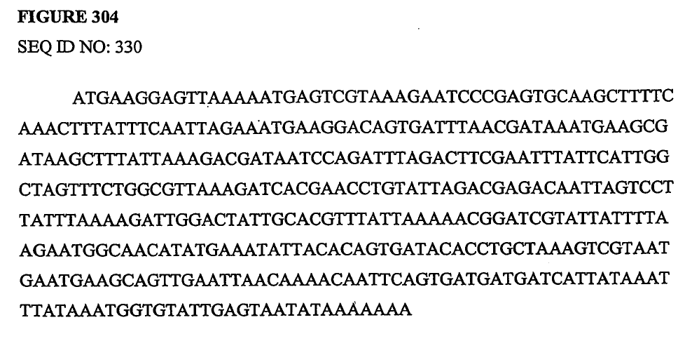

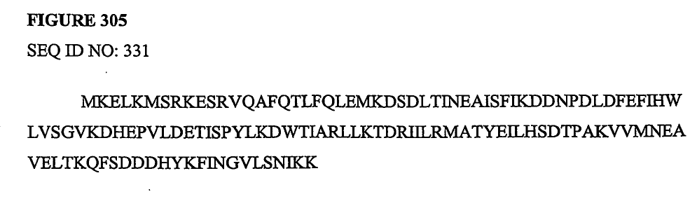

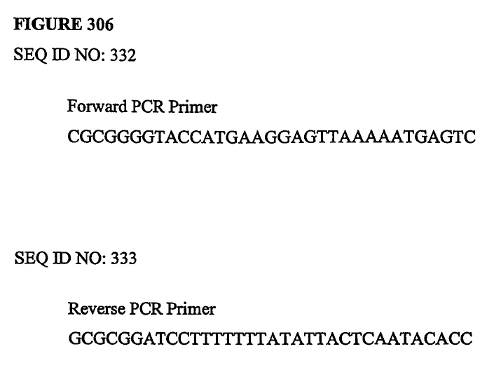

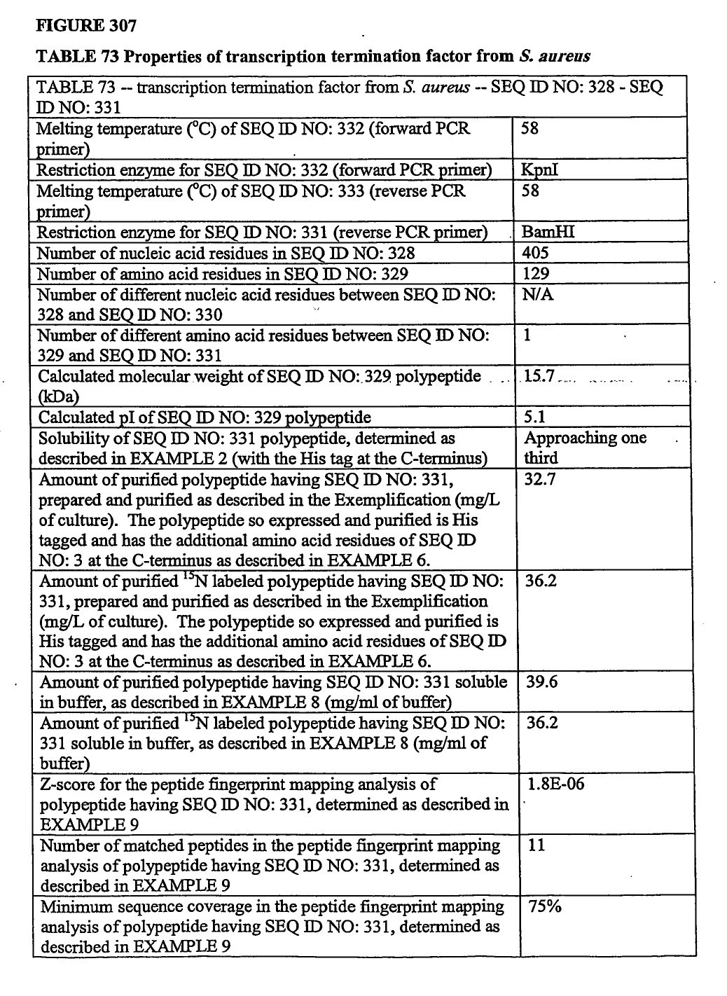

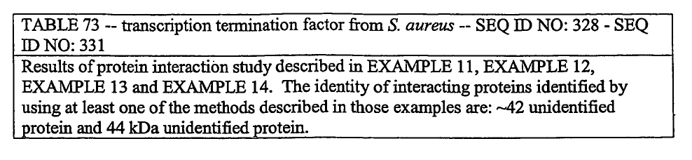

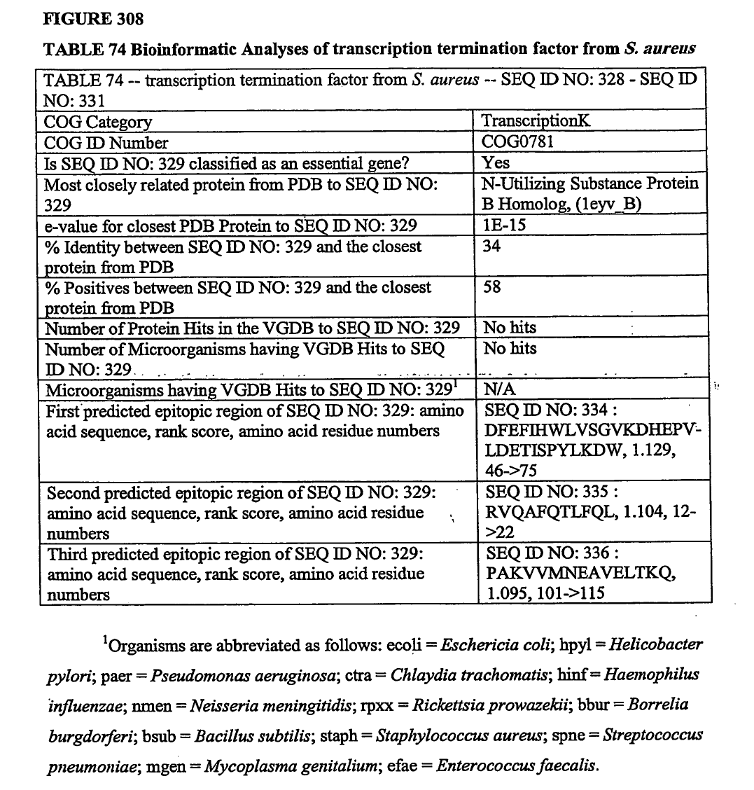

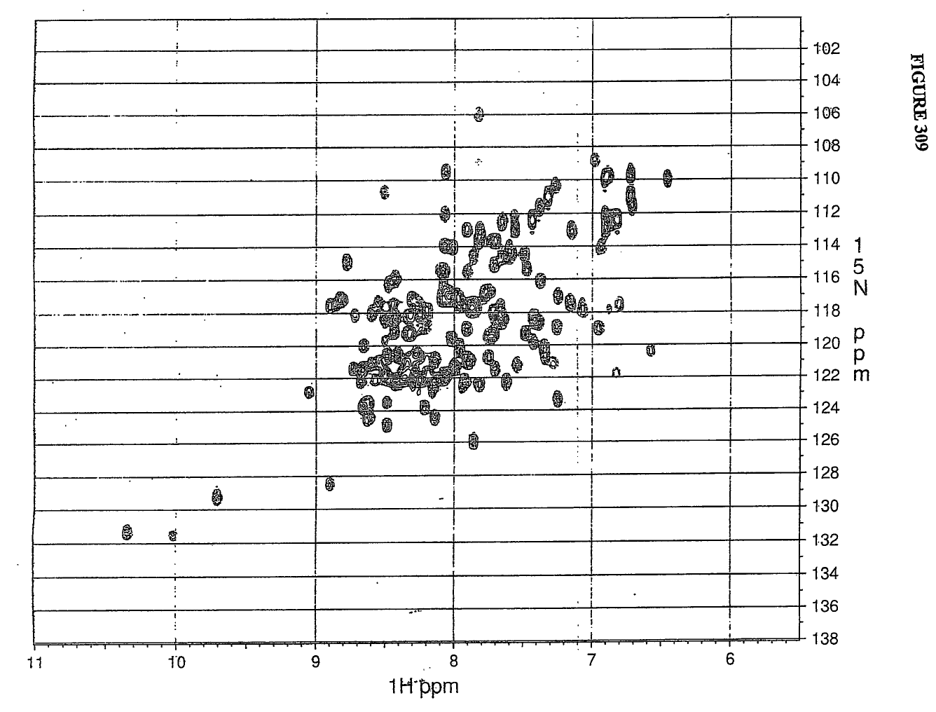

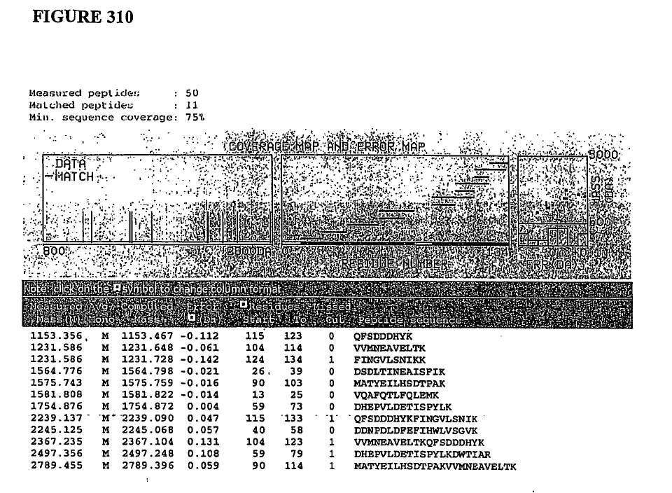

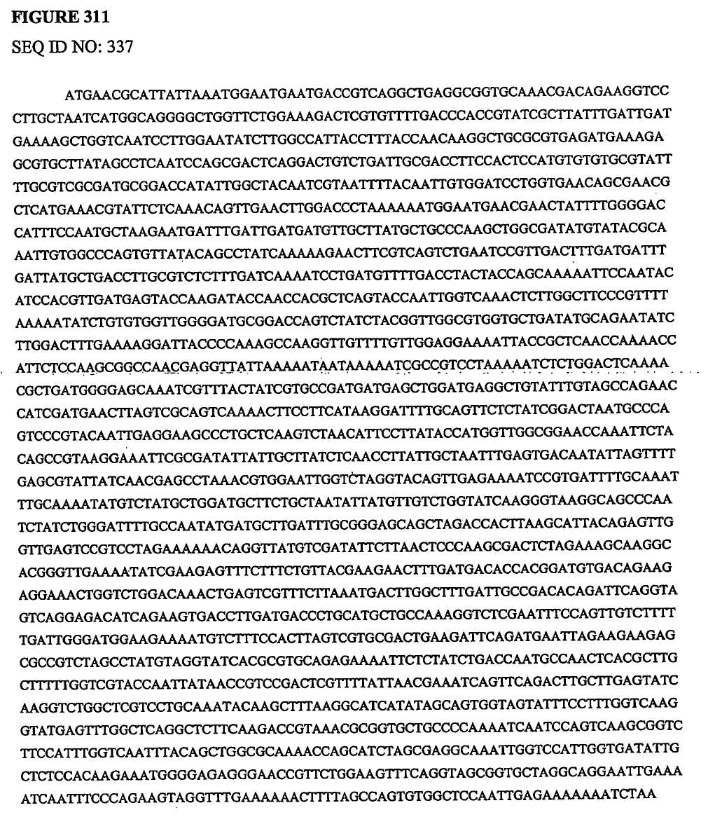

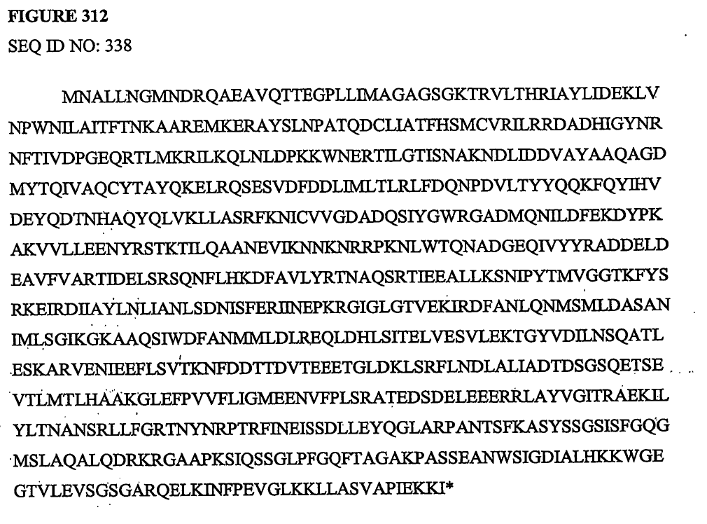

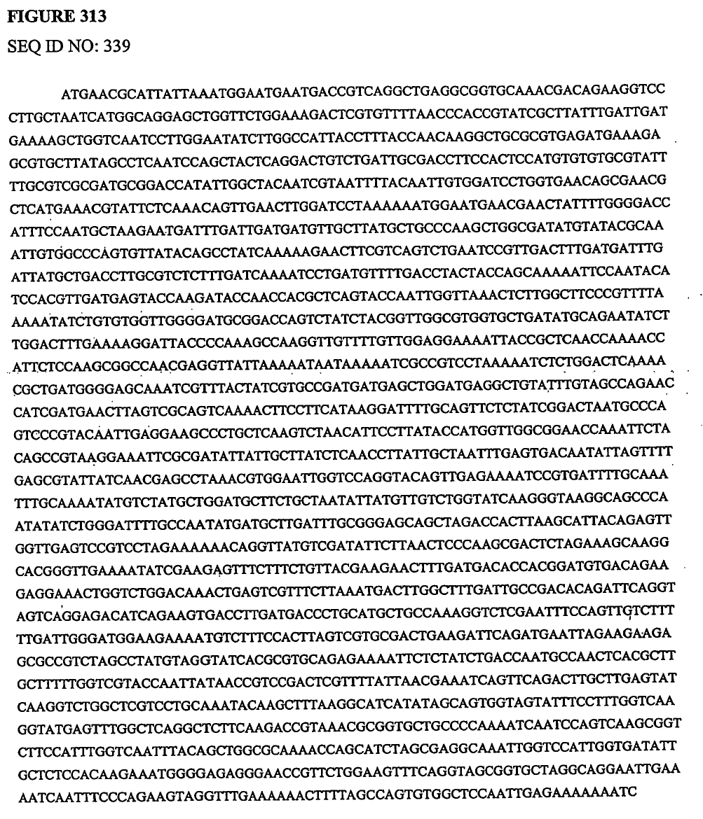

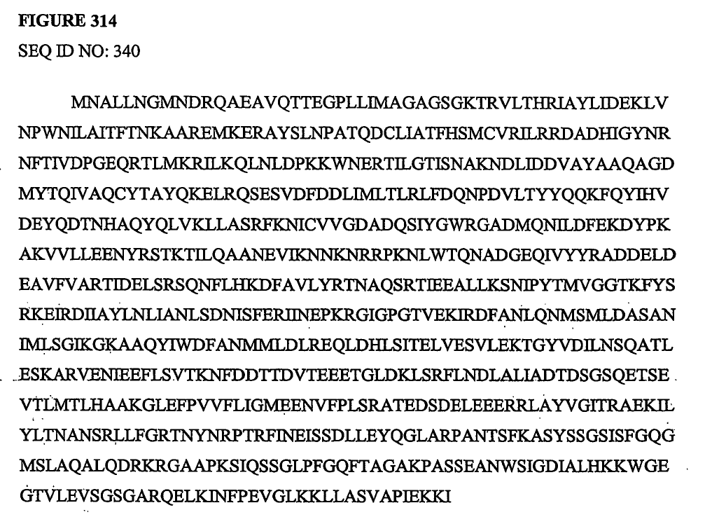

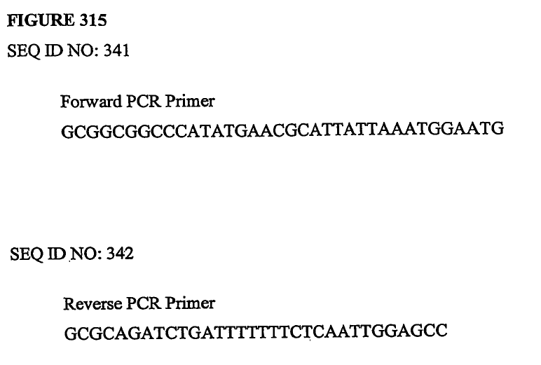

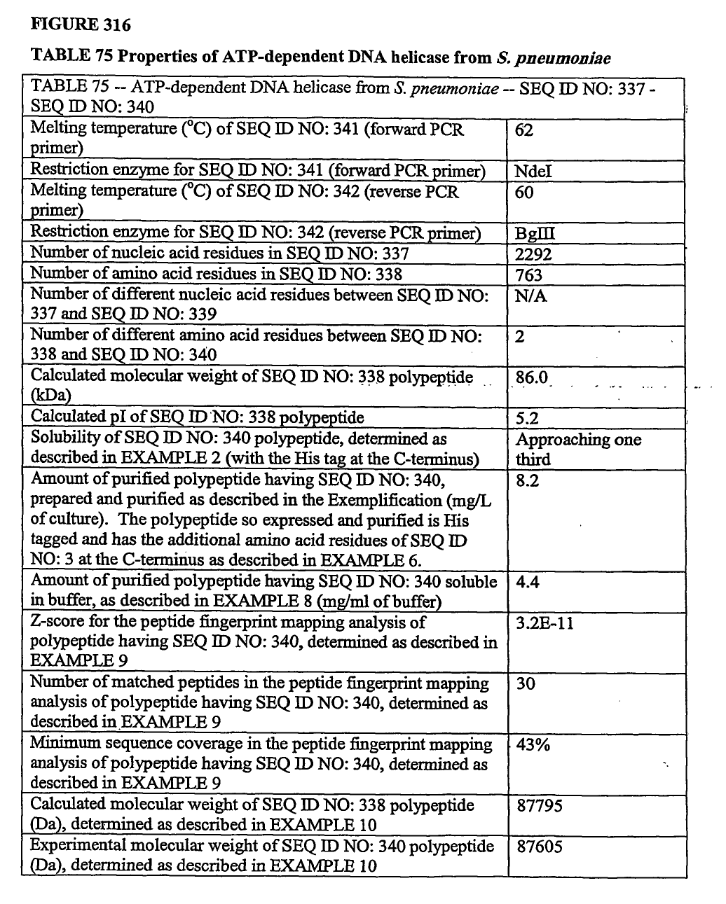

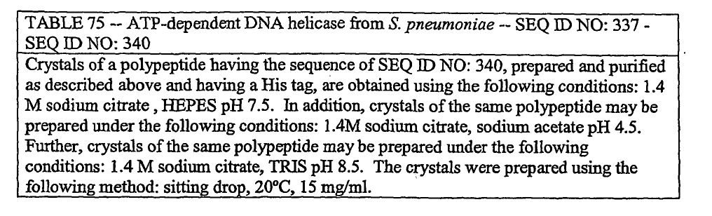

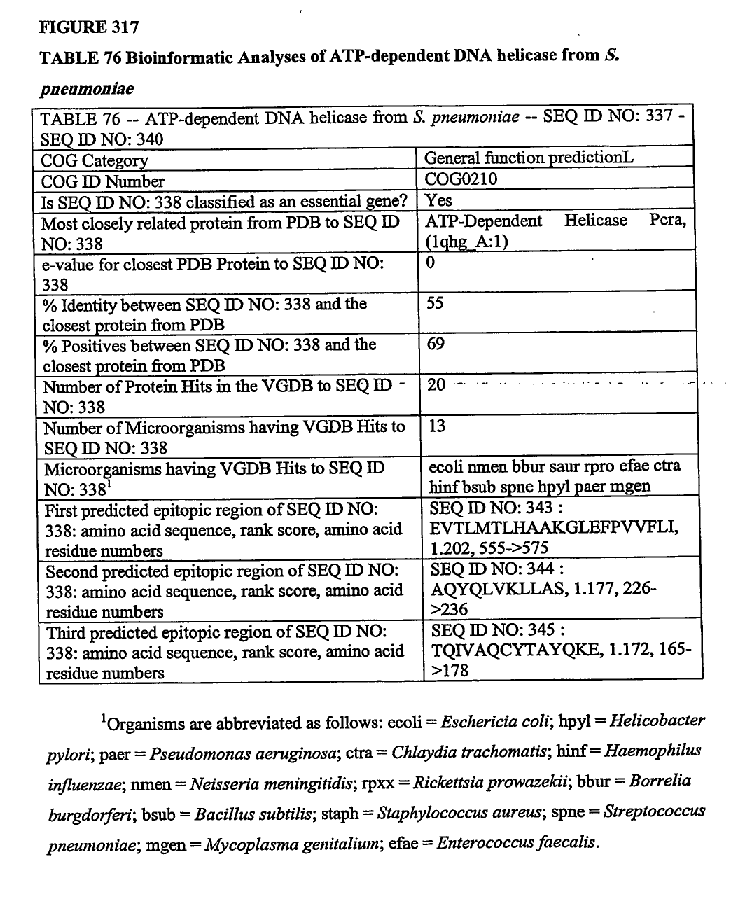

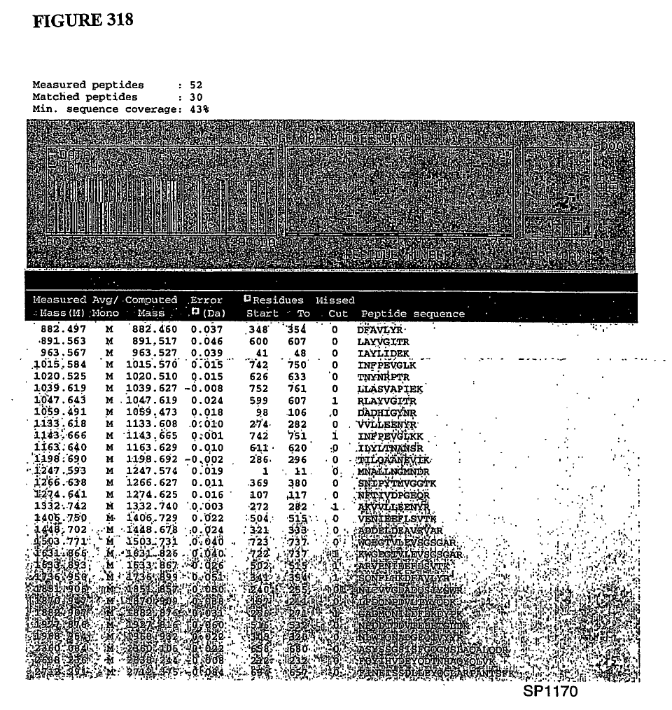

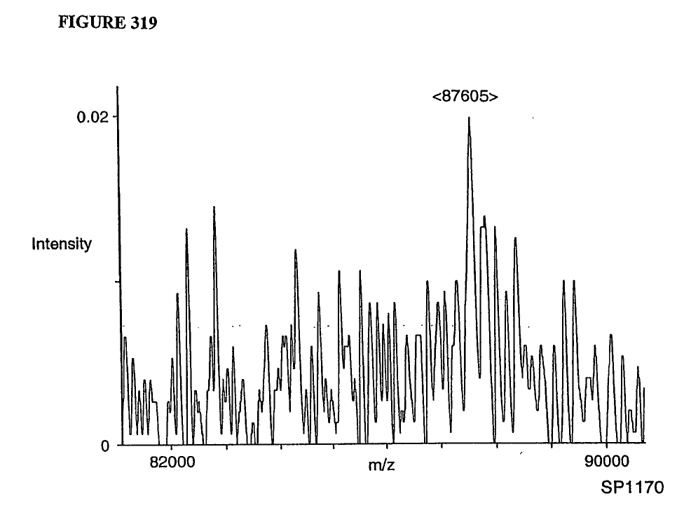

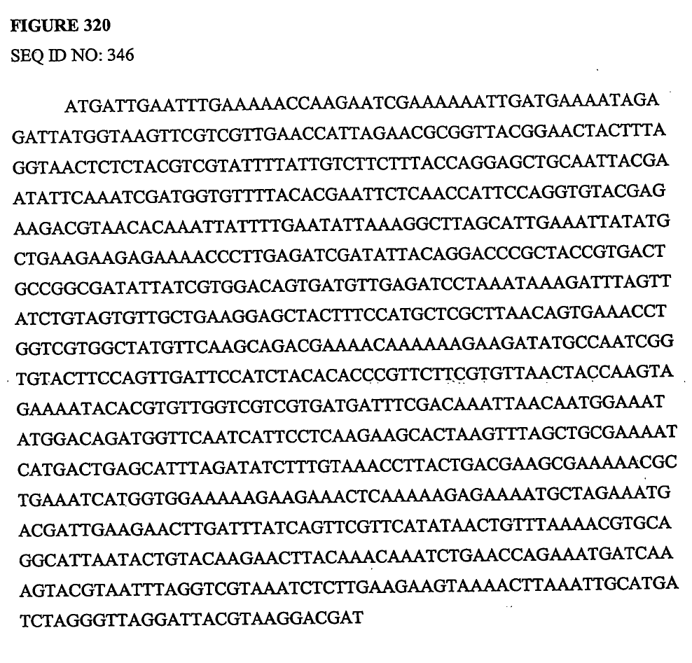

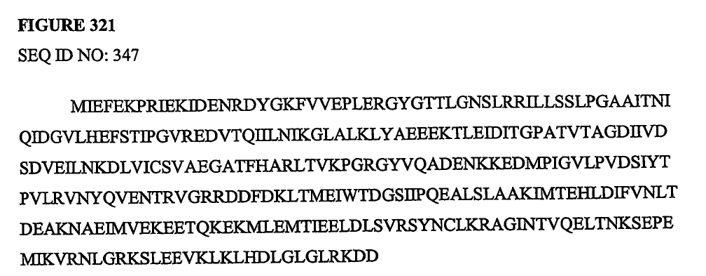

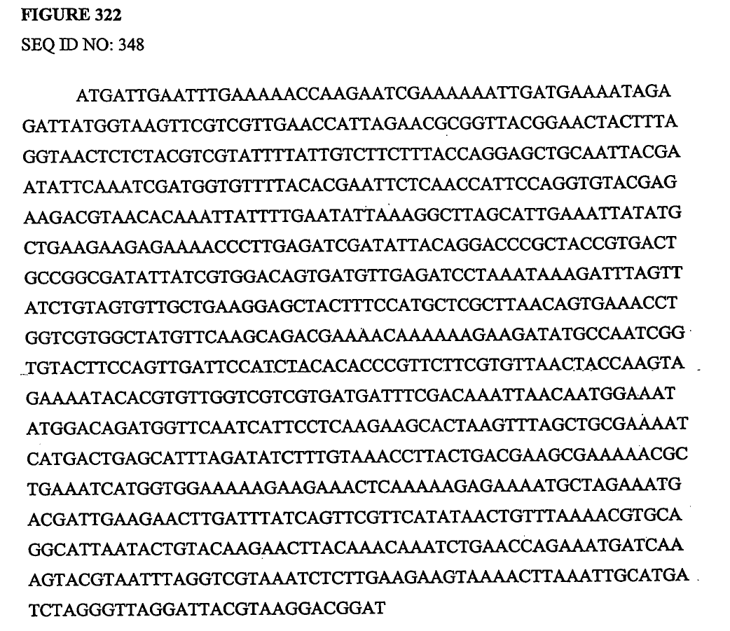

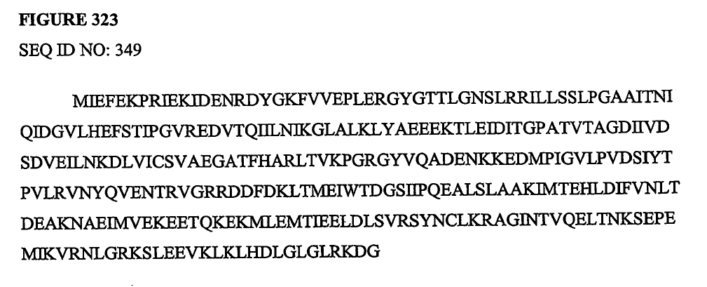

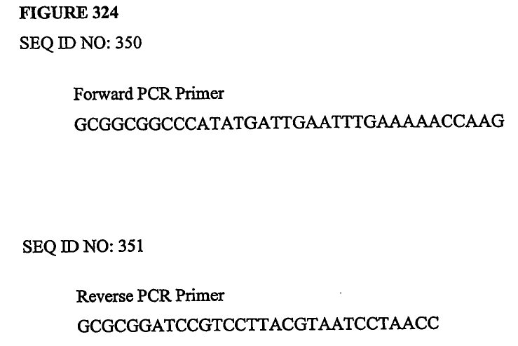

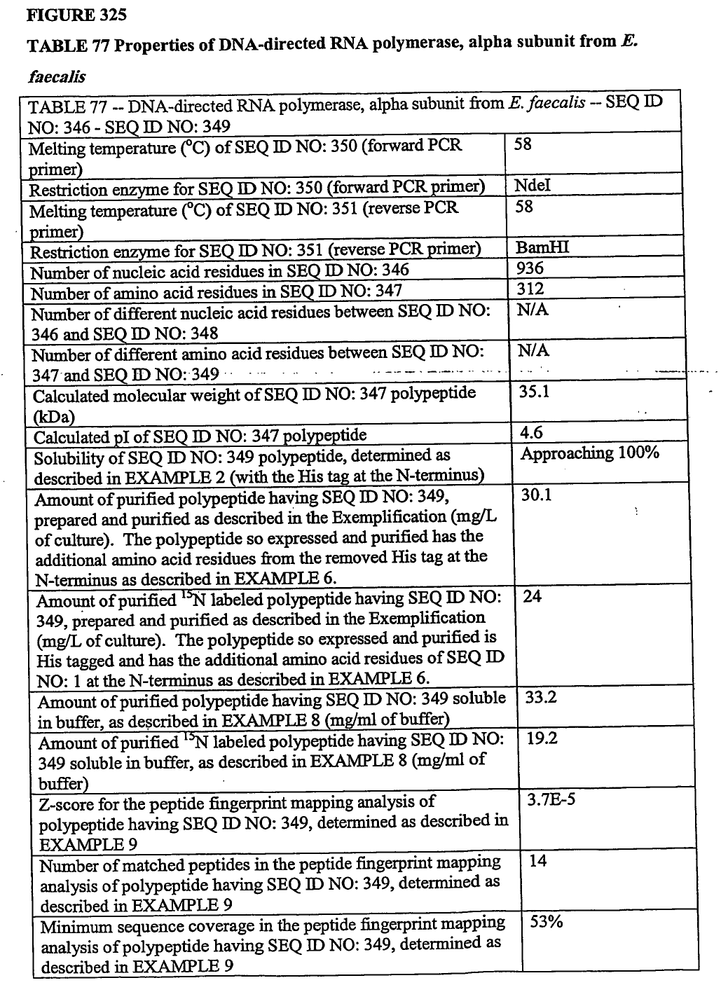

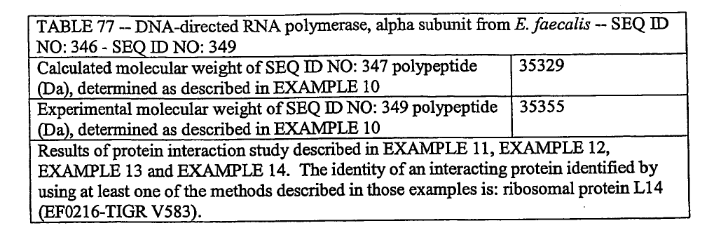

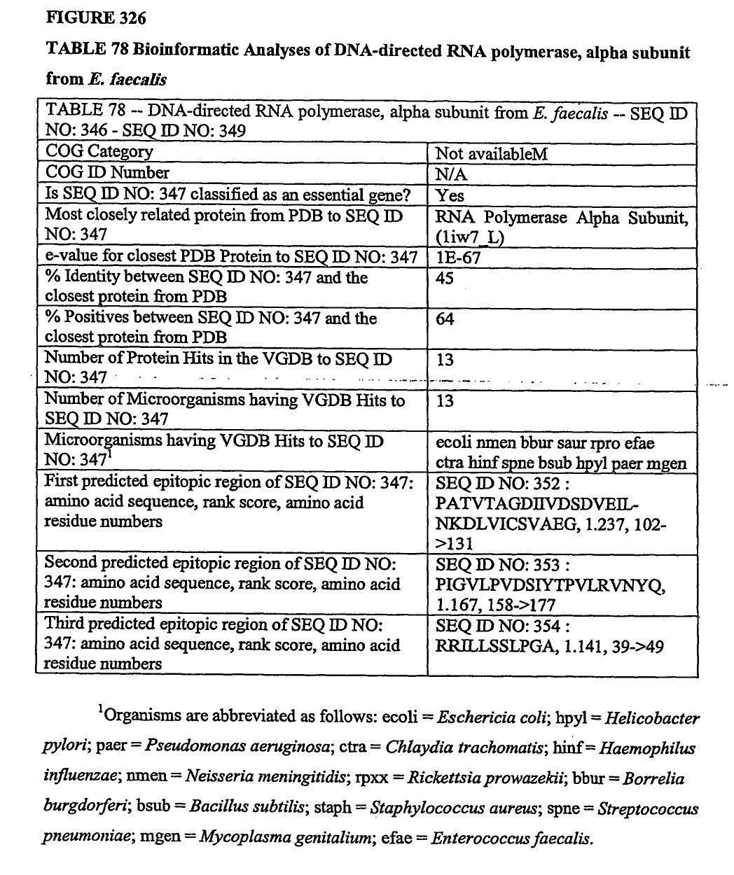

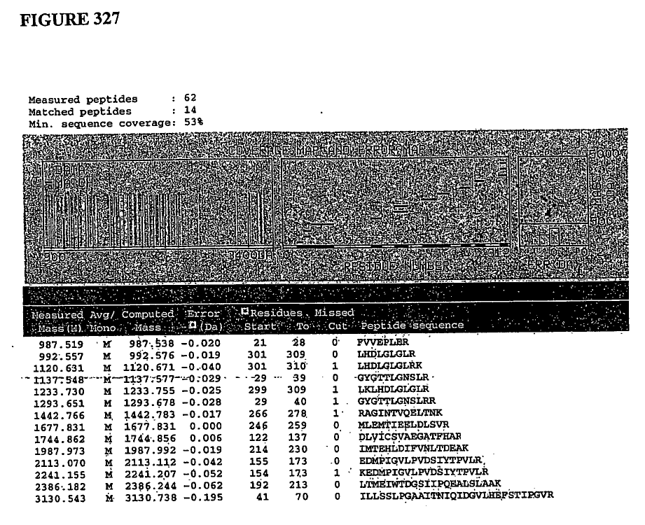

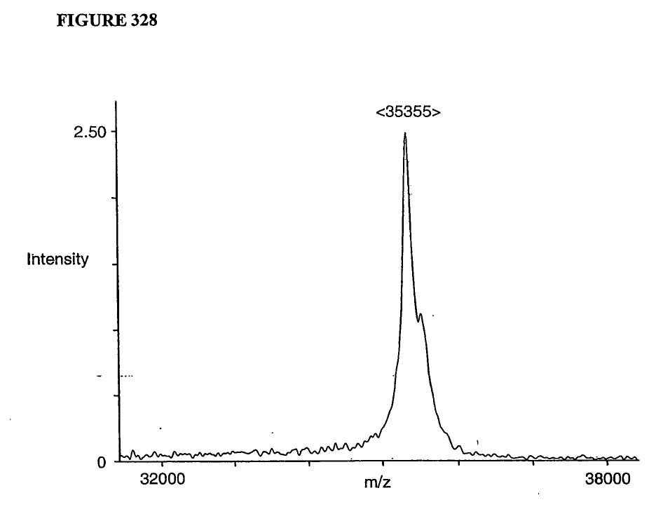

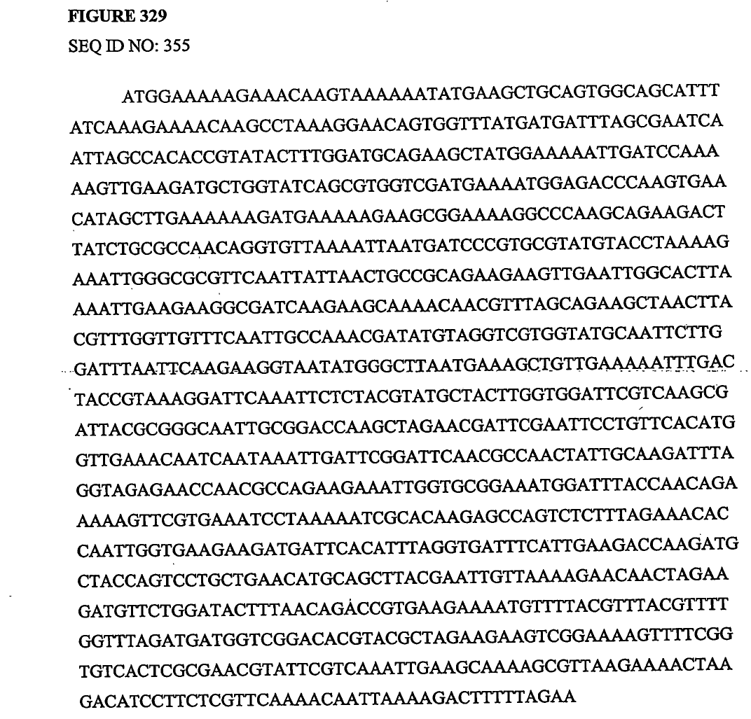

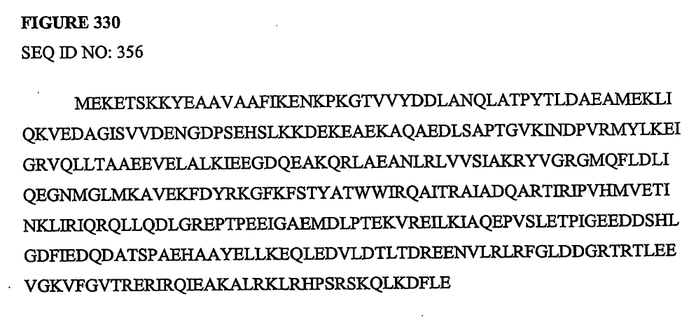

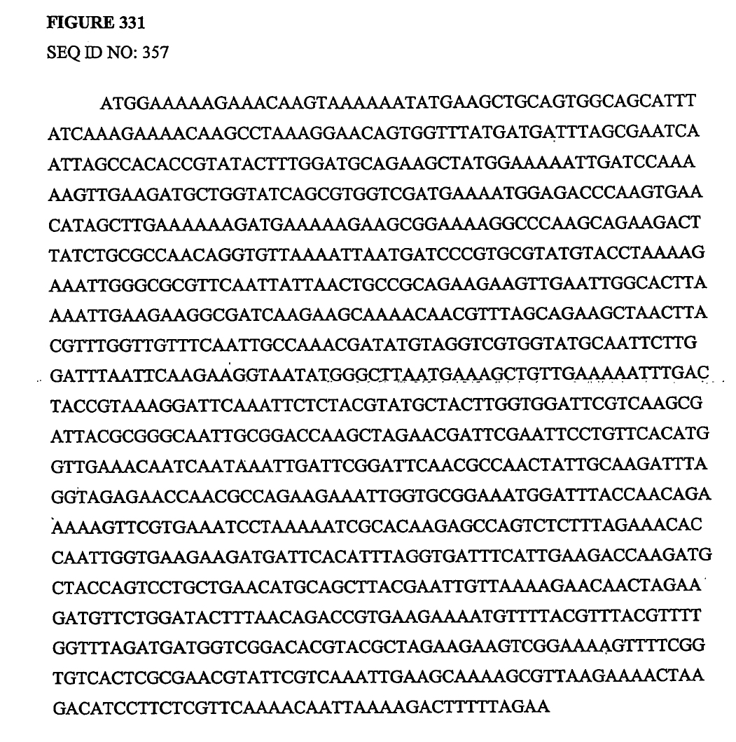

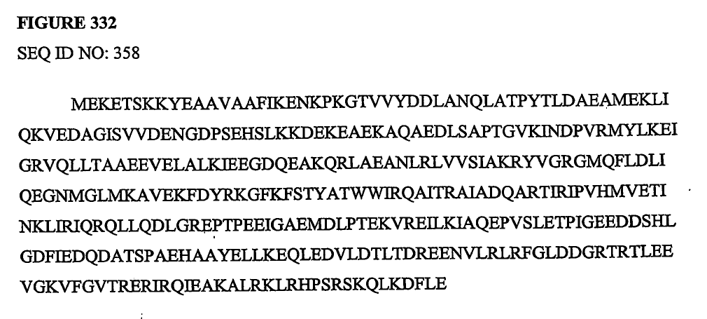

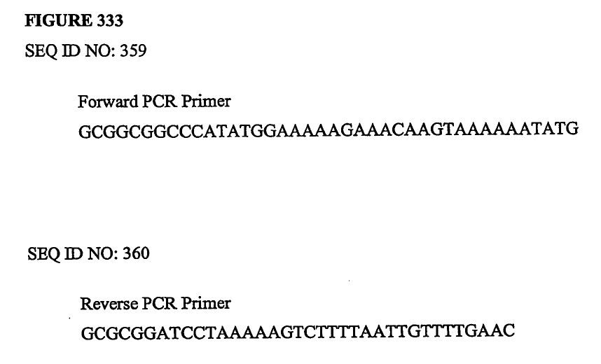

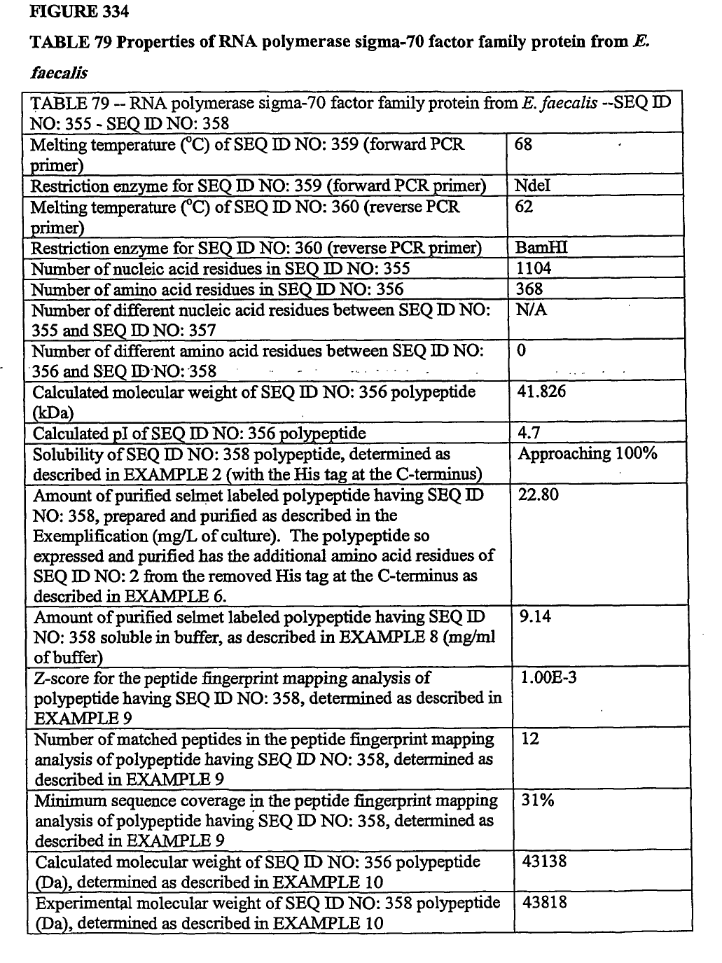

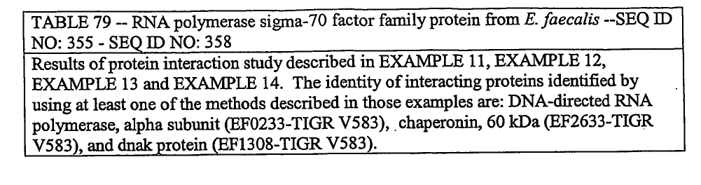

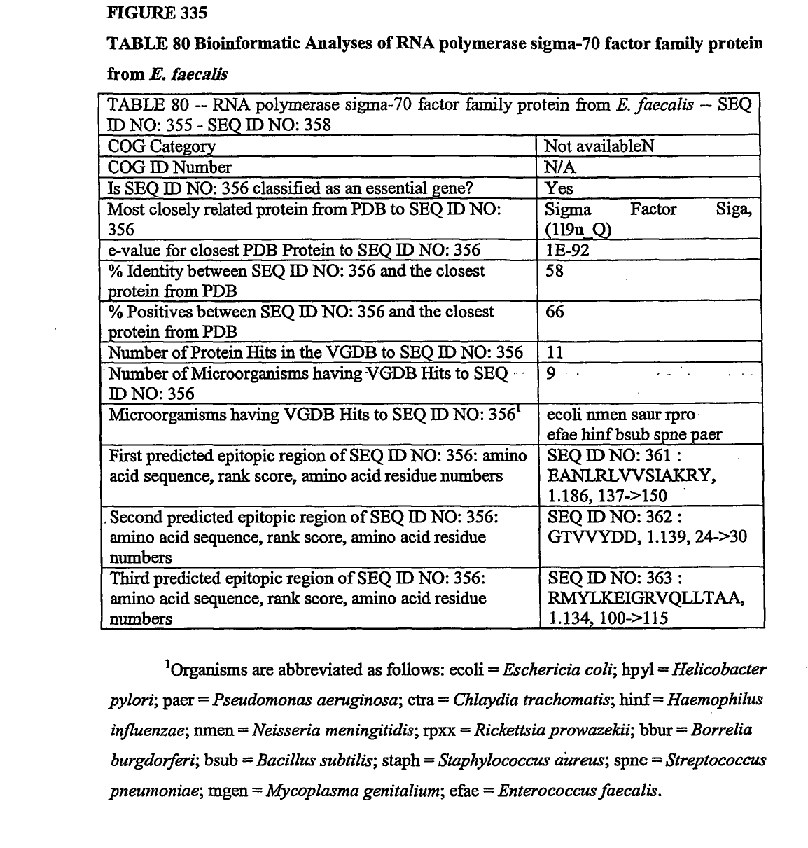

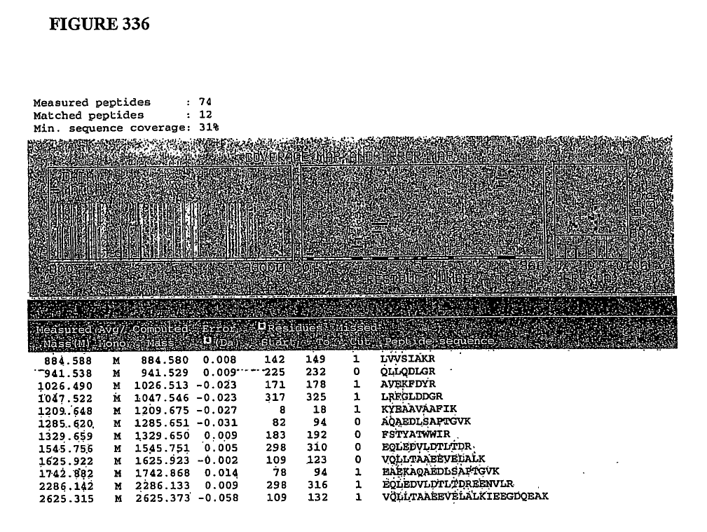

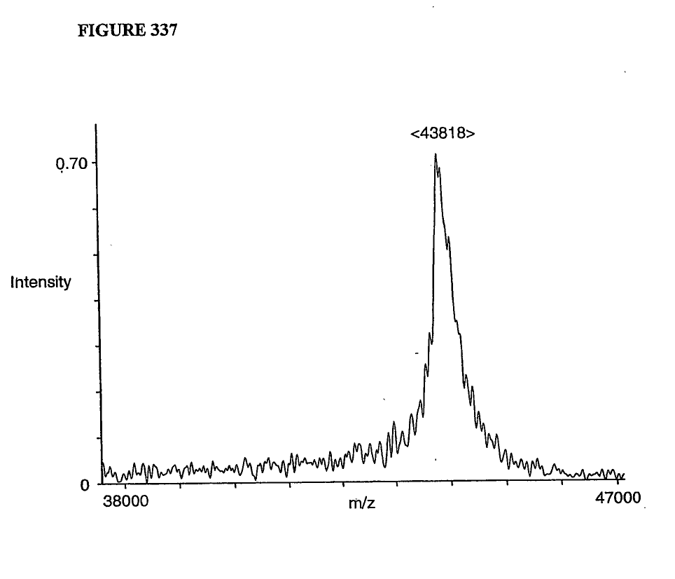

### DETAILED DESCRIPTION OF THE INVENTION

1. Definitions

For convenience, certain terms employed in the specification, examples, and appended claims are collected here. Unless defined otherwise, all technical and scientific terms used herein have the same meaning as commonly understood by one of ordinary skill in the art to which this invention belongs.

The articles “a” and “an” are used herein to refer to one or to more than one (i.e., to at least one) of the grammatical object of the article. By way of example, “an element” means one element or more than one element.

The term “amino acid” is intended to embrace all molecules, whether natural or synthetic, which include both an amino functionality and an acid functionality and capable of being included in a polymer of naturally-occurring amino acids. Exemplary amino acids include naturally-occurring amino acids; analogs, derivatives and congeners thereof; amino acid analogs having variant side chains; and all stereoisomers of any of any of the foregoing.

The term “binding” refers to an association, which may be a stable association, between two molecules, e.g., between a polypeptide of the invention and a binding partner, due to, for example, electrostatic, hydrophobic, ionic and/or hydrogen-bond interactions under physiological conditions.

A “comparison window,” as used herein, refers to a conceptual segment of at least 20 contiguous amino acid positions wherein a protein sequence may be compared to a reference sequence of at least 20 contiguous amino acids and wherein the portion of the protein sequence in the comparison window may comprise additions or deletions (i.e., gaps) of 20 percent or less as compared to the reference sequence (which does not comprise additions or deletions) for optimal alignment of the two sequences. Optimal alignment of sequences for aligning a comparison window may be conducted by the local homology algorithm of Smith and Waterman (1981) Adv. Appl. Math. 2: 482, by the homology alignment algorithm of Needleman and Wunsch (1970) J. Mol. Biol. 48: 443, by the search for similarity method of Pearson and Lipman (1988) Proc. Natl. Acad. Sci. (U.S.A.) 85: 2444, by computerized implementations of these algorithms (GAP, BESTFIT, FASTA, and TFASTA in the Wisconsin Genetics Software Package Release 7.0, Genetics Computer Group, 575 Science Dr., Madison, Wis.), or by inspection, and the best alignment (i.e., resulting in the highest percentage of homology over the comparison window) generated by the various methods may be identified.

The term “complex” refers to an association between at least two moieties (e.g. chemical or biochemical) that have an affinity for one another. Examples of complexes include associations between antigen/antibodies, lectin/avidin, target polynucleotide/probe oligonucleotide, antibody/anti-antibody, receptor/ligand, enzyme/ligand, polypeptide/polypeptide, polypeptide/polynucleotide, polypeptide/co-factor, polypeptide/substrate, polypeptide/inhibitor, polypeptide/small molecule, and the like. “Member of a complex” refers to one moiety of the complex, such as an antigen or ligand. “Protein complex” or “polypeptide complex” refers to a complex comprising at least one polypeptide.

The term “conserved residue” refers to an amino acid that is a member of a group of amino acids having certain common properties. The term “conservative amino acid substitution” refers to the substitution (conceptually or otherwise) of an amino acid from one such group with a different amino acid from the same group. A functional way to define common properties between individual amino acids is to analyze the normalized frequencies of amino acid changes between corresponding proteins of homologous organisms (Schulz, G. E. and R. H. Schirmer., Principles of Protein Structure, Springer-Verlag). According to such analyses, groups of amino acids may be defined where amino acids within a group exchange preferentially with each other, and therefore resemble each other most in their impact on the overall protein structure (Schulz, G. E. and R. H. Schirmer, Principles of Protein Structure, Springer-Verlag). One example of a set of amino acid groups defined in this manner include: (i) a charged group, consisting of Glu and Asp, Lys, Arg and His, (ii) a positively-charged group, consisting of Lys, Arg and His, (iii) a negatively-charged group, consisting of Glu and Asp, (iv) an aromatic group, consisting of Phe, Tyr and Trp, (v) a nitrogen ring group, consisting of His and Trp, (vi) a large aliphatic nonpolar group, consisting of Val, Leu and Ile, (vii) a slightly-polar group, consisting of Met and Cys, (viii) a small-residue group, consisting of Ser, Thr, Asp, Asn, Gly, Ala, Glu, Gln and Pro, (ix) an aliphatic group consisting of Val, Leu, Ile, Met and Cys, and (x) a small hydroxyl group consisting of Ser and Thr.

The term “domain”, when used in connection with a polypeptide, refers to a specific region within such polypeptide that comprises a particular structure or mediates a particular function. In the typical case, a domain of a polypeptide of the invention is a fragment of the polypeptide. In certain instances, a domain is a structurally stable domain, as evidenced, for example, by mass spectroscopy, or by the fact that a modulator may bind to a druggable region of the domain.

The term “druggable region”, when used in reference to a polypeptide, nucleic acid, complex and the like, refers to a region of the molecule which is a target or is a likely target for binding a modulator. For a polypeptide, a druggable region generally refers to a region wherein several amino acids of a polypeptide would be capable of interacting with a modulator or other molecule. For a polypeptide or complex thereof, exemplary druggable regions including binding pockets and sites, enzymatic active sites, interfaces between domains of a polypeptide or complex, surface grooves or contours or surfaces of a polypeptide or complex which are capable of participating in interactions with another molecule. In certain instances, the interacting molecule is another polypeptide, which may be naturally-occurring. In other instances, the druggable region is on the surface of the molecule.

Druggable regions may be described and characterized in a number of ways. For example, a druggable region may be characterized by some or all of the amino acids that make up the region, or the backbone atoms thereof, or the side chain atoms thereof (optionally with or without the Cα atoms). Alternatively, in certain instances, the volume of a druggable region corresponds to that of a carbon based molecule of at least about 200 amu and often up to about 800 amu. In other instances, it will be appreciated that the volume of such region may correspond to a molecule of at least about 600 amu and often up to about 1600 amu or more.

Alternatively, a druggable region may be characterized by comparison to other regions on the same or other molecules. For example, the term “affinity region” refers to a druggable region on a molecule (such as a polypeptide of the invention) that is present in several other molecules, in so much as the structures of the same affinity regions are sufficiently the same so that they are expected to bind the same or related structural analogs. An example of an affinity region is an ATP-binding site of a protein kinase that is found in several protein kinases (whether or not of the same origin). The term “selectivity region” refers to a druggable region of a molecule that may not be found on other molecules, in so much as the structures of different selectivity regions are sufficiently different so that they are not expected to bind the same or related structural analogs. An exemplary selectivity region is a catalytic domain of a protein kinase that exhibits specificity for one substrate. In certain instances, a single modulator may bind to the same affinity region across a number of proteins that have a substantially similar biological function, whereas the same modulator may bind to only one selectivity region of one of those proteins.

Continuing with examples of different druggable regions, the term “undesired region” refers to a druggable region of a molecule that upon interacting with another molecule results in an undesirable affect. For example, a binding site that oxidizes the interacting molecule (such as P-450 activity) and thereby results in increased toxicity for the oxidized molecule may be deemed a “undesired region”. Other examples of potential undesired regions includes regions that upon interaction with a drug decrease the membrane permeability of the drug, increase the excretion of the drug, or increase the blood brain transport of the drug. It may be the case that, in certain circumstances, an undesired region will no longer be deemed an undesired region because the affect of the region will be favorable, e.g., a drug intended to treat a brain condition would benefit from interacting with a region that resulted in increased blood brain transport, whereas the same region could be deemed undesirable for drugs that were not intended to be delivered to the brain.

When used in reference to a druggable region, the “selectivity” or “specificity’ of a molecule such as a modulator to a druggable region may be used to describe the binding between the molecule and a druggable region. For example, the selectivity of a modulator with respect to a druggable region may be expressed by comparison to another modulator, using the respective values of K_{d} (i.e., the dissociation constants for each modulator-druggable region complex) or, in cases where a biological effect is observed below the K_{d}, the ratio of the respective EC₅₀'s (i.e., the concentrations that produce 50% of the maximum response for the modulator interacting with each druggable region).

A “fusion protein” or “fusion polypeptide” refers to a chimeric protein as that term is known in the art and may be constructed using methods known in the art. In many examples of fusion proteins, there are two different polypeptide sequences, and in certain cases, there may be more. The sequences may be linked in frame. A fusion protein may include a domain which is found (albeit in a different protein) in an organism which also expresses the first protein, or it may be an “interspecies”, “intergenic”, etc. fusion expressed by different kinds of organisms. In various embodiments, the fusion polypeptide may comprise one or more amino acid sequences linked to a first polypeptide. In the case where more than one amino acid sequence is fused to a first polypeptide, the fusion sequences may be multiple copies of the same sequence, or alternatively, may be different amino acid sequences. The fusion polypeptides may be fused to the N-terminus, the C-terminus, or the N- and C-terminus of the first polypeptide. Exemplary fusion proteins include polypeptides comprising a glutathione S-transferase tag (GST-tag), histidine tag (His-tag), an immunoglobulin domain or an immunoglobulin binding domain.

The term “gene” refers to a nucleic acid comprising an open reading frame encoding a polypeptide having exon sequences and optionally intron sequences. The term “intron” refers to a DNA sequence present in a given gene which is not translated into protein and is generally found between exons.

The term “having substantially similar biological activity”, when used in reference to two polypeptides, refers to a biological activity of a first polypeptide which is substantially similar to at least one of the biological activities of a second polypeptide. A substantially similar biological activity means that the polypeptides carry out a similar function, e.g., a similar enzymatic reaction or a similar physiological process, etc. For example, two homologous proteins may have a substantially similar biological activity if they are involved in a similar enzymatic reaction, e.g., they are both kinases which catalyze phosphorylation of a substrate polypeptide, however, they may phosphorylate different regions on the same protein substrate or different substrate proteins altogether. Alternatively, two homologous proteins may also have a substantially similar biological activity if they are both involved in a similar physiological process, e.g., transcription. For example, two proteins may be transcription factors, however, they may bind to different DNA sequences or bind to different polypeptide interactors. Substantially similar biological activities may also be associated with proteins carrying out a similar structural role, for example, two membrane proteins.

The term “isolated polypeptide” refers to a polypeptide, in certain embodiments prepared from recombinant DNA or RNA, or of synthetic origin, or some combination thereof, which (1) is not associated with proteins that it is normally found with in nature, (2) is isolated from the cell in which it normally occurs, (3) is isolated free of other proteins from the same cellular source, (4) is expressed by a cell from a different species, or (5) does not occur in nature.

The term “isolated nucleic acid” refers to a polynucleotide of genomic, cDNA, or synthetic origin or some combination there of, which (1) is not associated with the cell in which the “isolated nucleic acid” is found in nature, or (2) is operably linked to a polynucleotide to which it is not linked in nature.

The terms “label” or “labeled” refer to incorporation or attachment, optionally covalently or non-covalently, of a detectable marker into a molecule, such as a polypeptide. Various methods of labeling polypeptides are known in the art and may be used. Examples of labels for polypeptides include, but are not limited to, the following: radioisotopes, fluorescent labels, heavy atoms, enzymatic labels or reporter genes, chemiluminescent groups, biotinyl groups, predetermined polypeptide epitopes recognized by a secondary reporter (e.g., leucine zipper pair sequences, binding sites for secondary antibodies, metal binding domains, epitope tags). Examples and use of such labels are described in more detail below. In some embodiments, labels are attached by spacer arms of various lengths to reduce potential steric hindrance.

The term “mammal” is known in the art, and exemplary mammals include humans, primates, bovines, porcines, canines, felines, and rodents (e.g., mice and rats).

The term “modulation”, when used in reference to a functional property or biological activity or process (e.g., enzyme activity or receptor binding), refers to the capacity to either up regulate (e.g., activate or stimulate), down regulate (e.g., inhibit or suppress) or otherwise change a quality of such property, activity or process. In certain instances, such regulation may be contingent on the occurrence of a specific event, such as activation of a signal transduction pathway, and/or may be manifest only in particular cell types.

The term “modulator” refers to a polypeptide, nucleic acid, macromolecule, complex, molecule, small molecule, compound, species or the like (naturally-occurring or non-naturally-occurring), or an extract made from biological materials such as bacteria, plants, fungi, or animal cells or tissues, that may be capable of causing modulation. Modulators may be evaluated for potential activity as inhibitors or activators (directly or indirectly) of a functional property, biological activity or process, or combination of them, (e.g., agonist, partial antagonist, partial agonist, inverse agonist, antagonist, anti-microbial agents, inhibitors of microbial infection or proliferation, and the like) by inclusion in assays. In such assays, many modulators may be screened at one time. The activity of a modulator may be known, unknown or partially known.

The term “motif” refers to an amino acid sequence that is commonly found in a protein of a particular structure or function. Typically, a consensus sequence is defined to represent a particular motif. The consensus sequence need not be strictly defined and may contain positions of variability, degeneracy, variability of length, etc. The consensus sequence may be used to search a database to identify other proteins that may have a similar structure or function due to the presence of the motif in its amino acid sequence. For example, on-line databases may be searched with a consensus sequence in order to identify other proteins containing a particular motif. Various search algorithms and/or programs may be used, including FASTA, BLAST or ENTREZ. FASTA and BLAST are available as a part of the GCG sequence analysis package (University of Wisconsin, Madison, Wis.). ENTREZ is available through the National Center for Biotechnology Information, National Library of Medicine, National Institutes of Health, Bethesda, Md.

The term “naturally-occurring”, as applied to an object, refers to the fact that an object may be found in nature. For example, a polypeptide or polynucleotide sequence that is present in an organism (including bacteria) that may be isolated from a source in nature and which has not been intentionally modified by man in the laboratory is naturally-occurring.

The term “nucleic acid” refers to a polymeric form of nucleotides, either ribonucleotides or deoxynucleotides or a modified form of either type of nucleotide. The terms should also be understood to include, as equivalents, analogs of either RNA or DNA made from nucleotide analogs, and, as applicable to the embodiment being described, single-stranded (such as sense or antisense) and double-stranded polynucleotides.

The term “nucleic acid of the invention” refers to a nucleic acid encoding a polypeptide of the invention, e.g., a nucleic acid comprising a sequence consisting of, or consisting essentially of, a subject nucleic acid sequence. A nucleic acid of the invention may comprise all, or a portion of, a subject nucleic acid sequence; a nucleotide sequence at least 60%, 70%, 80%, 90%, 95%, 96%, 97%, 98% or 99% identical to a subject nucleic acid sequence; a nucleotide sequence that hybridizes under stringent conditions to a subject nucleic acid sequence; nucleotide sequences encoding polypeptides that are functionally equivalent to polypeptides of the invention; nucleotide sequences encoding polypeptides at least about 60%, 70%, 80%, 85%, 90%, 95%, 98%, 99% homologous or identical with a subject amino acid sequence; nucleotide sequences encoding polypeptides having an activity of a polypeptide of the invention and having at least about 60%, 70%, 80%, 85%, 90%, 95%, 98%, 99% or more homology or identity with a subject amino acid sequence; nucleotide sequences that differ by 1 to about 2, 3, 5, 7, 10, 15, 20, 30, 50, 75 or more nucleotide substitutions, additions or deletions, such as allelic variants, of a subject nucleic acid sequence; nucleic acids derived from and evolutionarily related to a subject nucleic acid sequence; and complements of, and nucleotide sequences resulting from the degeneracy of the genetic code, for all of the foregoing and other nucleic acids of the invention. Nucleic acids of the invention also include homologs, e.g., orthologs and paralogs, of a subject nucleic acid sequence and also variants of a subject nucleic acid sequence which have been codon optimized for expression in a particular organism (e.g., host cell).

The term “operably linked”, when describing the relationship between two nucleic acid regions, refers to a juxtaposition wherein the regions are in a relationship permitting them to function in their intended manner. For example, a control sequence “operably linked” to a coding sequence is ligated in such a way that expression of the coding sequence is achieved under conditions compatible with the control sequences, such as when the appropriate molecules (e.g., inducers and polymerases) are bound to the control or regulatory sequence(s).

The term “phenotype” refers to the entire physical, biochemical, and physiological makeup of a cell, e.g., having any one trait or any group of traits.

The term “polypeptide”, and the terms “protein” and “peptide” which are used interchangeably herein, refers to a polymer of amino acids. Exemplary polypeptides include gene products, naturally-occurring proteins, homologs, orthologs, paralogs, fragments, and other equivalents, variants and analogs of the foregoing.

The terms “polypeptide fragment” or “fragment”, when used in reference to a reference polypeptide, refers to a polypeptide in which amino acid residues are deleted as compared to the reference polypeptide itself, but where the remaining amino acid sequence is usually identical to the corresponding positions in the reference polypeptide. Such deletions may occur at the amino-terminus or carboxy-terminus of the reference polypeptide, or alternatively both. Fragments typically are at least 5, 6, 8 or 10 amino acids long, at least 14 amino acids long, at least 20, 30, 40 or 50 amino acids long, at least 75 amino acids long, or at least 100, 150, 200, 300, 500 or more amino acids long. A fragment can retain one or more of the biological activities of the reference polypeptide. In certain embodiments, a fragment may comprise a druggable region, and optionally additional amino acids on one or both sides of the druggable region, which additional amino acids may number from 5, 10, 15, 20, 30, 40, 50, or up to 100 or more residues. Further, fragments can include a sub-fragment of a specific region, which sub-fragment retains a function of the region from which it is derived. In another embodiment, a fragment may have immunogenic properties.

The term “polypeptide of the invention” refers to a polypeptide comprising a subject amino acid sequence, or an equivalent or fragment thereof, e.g., a polypeptide comprising a sequence consisting of, or consisting essentially of, a subject amino acid sequence. Polypeptides of the invention include polypeptides comprising all or a portion of a subject amino acid sequence; a subject amino acid sequence with 1 to about 2, 3, 5, 7, 10, 15, 20, 30, 50, 75 or more conservative amino acid substitutions; an amino acid sequence that is at least 60%, 70%, 80%, 90%, 95%, 96%, 97%, 98%, or 99% identical to a subject amino acid sequence; and functional fragments thereof. Polypeptides of the invention also include homologs, e.g., orthologs and paralogs, of a subject amino acid sequence.

The term “purified” refers to an object species that is the predominant species present (i.e., on a molar basis it is more abundant than any other individual species in the composition). A “purified fraction” is a composition wherein the object species comprises at least about 50 percent (on a molar basis) of all species present. In making the determination of the purity of a species in solution or dispersion, the solvent or matrix in which the species is dissolved or dispersed is usually not included in such determination; instead, only the species (including the one of interest) dissolved or dispersed are taken into account. Generally, a purified composition will have one species that comprises more than about 80 percent of all species present in the composition, more than about 85%, 90%, 95%, 99% or more of all species present. The object species may be purified to essential homogeneity (contaminant species cannot be detected in the composition by conventional detection methods) wherein the composition consists essentially of a single species. A skilled artisan may purify a polypeptide of the invention using standard techniques for protein purification in light of the teachings herein. Purity of a polypeptide may be determined by a number of methods known to those of skill in the art, including for example, amino-terminal amino acid sequence analysis, gel electrophoresis, mass-spectrometry analysis and the methods described in the Exemplification section herein.

The terms “recombinant protein” or “recombinant polypeptide” refer to a polypeptide which is produced by recombinant DNA techniques. An example of such techniques includes the case when DNA encoding the expressed protein is inserted into a suitable expression vector which is in turn used to transform a host cell to produce the protein or polypeptide encoded by the DNA.

A “reference sequence” is a defined sequence used as a basis for a sequence comparison; a reference sequence may be a subset of a larger sequence, for example, as a segment of a full-length protein given in a sequence listing such as a subject amino acid sequence, or may comprise a complete protein sequence. Generally, a reference sequence is at least 200, 300 or 400 nucleotides in length, frequently at least 600 nucleotides in length, and often at least 800 nucleotides in length (or the protein equivalent if it is shorter or longer in length). Because two proteins may each (1) comprise a sequence (i.e., a portion of the complete protein sequence) that is similar between the two proteins, and (2) may further comprise a sequence that is divergent between the two proteins, sequence comparisons between two (or more) proteins are typically performed by comparing sequences of the two proteins over a “comparison window” to identify and compare local regions of sequence similarity.

The term “regulatory sequence” is a generic term used throughout the specification to refer to polynucleotide sequences, such as initiation signals, enhancers, regulators and promoters, that are necessary or desirable to affect the expression of coding and non-coding sequences to which they are operably linked. Exemplary regulatory sequences are described in Goeddel; Gene Expression Technology: Methods in Enzymology, Academic Press, San Diego, Calif. (1990), and include, for example, the early and late promoters of SV40, adenovirus or cytomegalovirus immediate early promoter, the lac system, the trp system, the TAC or TRC system, T7 promoter whose expression is directed by T7 RNA polymerase, the major operator and promoter regions of phage lambda, the control regions for fd coat protein, the promoter for 3-phosphoglycerate kinase or other glycolytic enzymes, the promoters of acid phosphatase, e.g., Pho5, the promoters of the yeast α-mating factors, the polyhedron promoter of the baculovirus system and other sequences known to control the expression of genes of prokaryotic or eukaryotic cells or their viruses, and various combinations thereof. The nature and use of such control sequences may differ depending upon the host organism. In prokaryotes, such regulatory sequences generally include promoter, ribosomal binding site, and transcription termination sequences. The term “regulatory sequence” is intended to include, at a minimum, components whose presence may influence expression, and may also include additional components whose presence is advantageous, for example, leader sequences and fusion partner sequences. In certain embodiments, transcription of a polynucleotide sequence is under the control of a promoter sequence (or other regulatory sequence) which controls the expression of the polynucleotide in a cell-type in which expression is intended. It will also be understood that the polynucleotide can be under the control of regulatory sequences which are the same or different from those sequences which control expression of the naturally-occurring form of the polynucleotide.

The term “reporter gene” refers to a nucleic acid comprising a nucleotide sequence encoding a protein that is readily detectable either by its presence or activity, including, but not limited to, luciferase, fluorescent protein (e.g., green fluorescent protein), chloramphenicol acetyl transferase, β-galactosidase, secreted placental alkaline phosphatase, p-lactamase, human growth hormone, and other secreted enzyme reporters. Generally, a reporter gene encodes a polypeptide not otherwise produced by the host cell, which is detectable by analysis of the cell(s), e.g., by the direct fluorometric, radioisotopic or spectrophotometric analysis of the cell(s) and preferably without the need to kill the cells for signal analysis. In certain instances, a reporter gene encodes an enzyme, which produces a change in fluorometric properties of the host cell, which is detectable by qualitative, quantitative or semiquantitative function or transcriptional activation. Exemplary enzymes include esterases, β-lactamase, phosphatases, peroxidases, proteases (tissue plasminogen activator or urokinase) and other enzymes whose function may be detected by appropriate chromogenic or fluorogenic substrates known to those skilled in the art or developed in the future.

The term “sequence homology” refers to the proportion of base matches between two nucleic acid sequences or the proportion of amino acid matches between two amino acid sequences. When sequence homology is expressed as a percentage, e.g., 50%, the percentage denotes the proportion of matches over the length of sequence from a desired sequence (e.g., SEQ ID NO: 1) that is compared to some other sequence. Gaps (in either of the two sequences) are permitted to maximize matching; gap lengths of 15 bases or less are usually used, 6 bases or less are used more frequently, with 2 bases or less used even more frequently. The term “sequence identity” means that sequences are identical (i.e., on a nucleotide-by-nucleotide basis for nucleic acids or amino acid-by-amino acid basis for polypeptides) over a window of comparison. The term “percentage of sequence identity” is calculated by comparing two optimally aligned sequences over the comparison window, determining the number of positions at which the identical amino acids occurs in both sequences to yield the number of matched positions, dividing the number of matched positions by the total number of positions in the comparison window, and multiplying the result by 100 to yield the percentage of sequence identity. Methods to calculate sequence identity are known to those of skill in the art and described in further detail below.

The term “small molecule” refers to a compound, which has a molecular weight of less than about 5 kD, less than about 2.5 kD, less than about 1.5 kD, or less than about 0.9 kD. Small molecules may be, for example, nucleic acids, peptides, polypeptides, peptide nucleic acids, peptidomimetics, carbohydrates, lipids or other organic (carbon containing) or inorganic molecules. Many pharmaceutical companies have extensive libraries of chemical and/or biological mixtures, often fungal, bacterial, or algal extracts, which can be screened with any of the assays of the invention. The term “small organic molecule” refers to a small molecule that is often identified as being an organic or medicinal compound, and does not include molecules that are exclusively nucleic acids, peptides or polypeptides.

The term “soluble” as used herein with reference to a polypeptide of the invention or other protein, means that upon expression in cell culture, at least some portion of the polypeptide or protein expressed remains in the cytoplasmic fraction of the cell and does not fractionate with the cellular debris upon lysis and centrifugation of the lysate. Solubility of a polypeptide may be increased by a variety of art recognized methods, including fusion to a heterologous amino acid sequence, deletion of amino acid residues, amino acid substitution (e.g., enriching the sequence with amino acid residues having hydrophilic side chains), and chemical modification (e.g., addition of hydrophilic groups). The solubility of polypeptides may be measured using a variety of art recognized techniques, including, dynamic light scattering to determine aggregation state, UV absorption, centrifugation to separate aggregated from non-aggregated material, and SDS gel electrophoresis (e.g., the amount of protein in the soluble fraction is compared to the amount of protein in the soluble and insoluble fractions combined). When expressed in a host cell, the polypeptides of the invention may be at least about 1%, 2%, 5%, 10%, 20%, 30%, 40%, 50%, 60%, 70%, 80%, 90% or more soluble, e.g., at least about 1%, 2%, 5%, 10%, 20%, 30%, 40%, 50%, 60%, 70%, 80%, 90% or more of the total amount of protein expressed in the cell is found in the cytoplasmic fraction. In certain embodiments, a one liter culture of cells expressing a polypeptide of the invention will produce at least about 0.1, 0.2, 0.5, 1, 2, 5, 10, 20, 30, 40, 50 milligrams or more of soluble protein. In an exemplary embodiment, a polypeptide of the invention is at least about 10% soluble and will produce at least about 1 milligram of protein from a one liter cell culture.

The term “specifically hybridizes” refers to detectable and specific nucleic acid binding. Polynucleotides, oligonucleotides and nucleic acids of the invention selectively hybridize to nucleic acid strands under hybridization and wash conditions that minimize appreciable amounts of detectable binding to nonspecific nucleic acids. Stringent conditions may be used to achieve selective hybridization conditions as known in the art and discussed herein. Generally, the nucleic acid sequence homology between the polynucleotides, oligonucleotides, and nucleic acids of the invention and a nucleic acid sequence of interest will be at least 30%, 40%, 50%, 60%, 70%, 80%, 85%, 90%, 95%, 98%, 99%, or more. In certain instances, hybridization and washing conditions are performed under stringent conditions according to conventional hybridization procedures and as described further herein.

The terms “stringent conditions” or “stringent hybridization conditions” refer to conditions which promote specific hydribization between two complementary polynucleotide strands so as to form a duplex. Stringent conditions may be selected to be about 5° C. lower than the thermal melting point (Tm) for a given polynucleotide duplex at a defined ionic strength and pH. The length of the complementary polynucleotide strands and their GC content will determine the Tm of the duplex, and thus the hybridization conditions necessary for obtaining a desired specificity of hybridization. The Tm is the temperature (under defined ionic strength and pH) at which 50% of the a polynucleotide sequence hybridizes to a perfectly matched complementary strand. In certain cases it may be desirable to increase the stringency of the hybridization conditions to be about equal to the Tm for a particular duplex.

A variety of techniques for estimating the Tm are available. Typically, G-C base pairs in a duplex are estimated to contribute about 3° C. to the Tm, while A-T base pairs are estimated to contribute about 2° C., up to a theoretical maximum of about 80-100° C. However, more sophisticated models of Tm are available in which G-C stacking interactions, solvent effects, the desired assay temperature and the like are taken into account. For example, probes can be designed to have a dissociation temperature (Td) of approximately 60° C., using the formula: Td=(((((3×#GC)+(2×#AT))×37)−562)/#bp)−5; where #GC, #AT, and #bp are the number of guanine-cytosine base pairs, the number of adenine-thymine base pairs, and the number of total base pairs, respectively, involved in the formation of the duplex.

Hybridization may be carried out in 5×SSC, 4×SSC, 3×SSC, 2×SSC, 1×SSC or 0.2×SSC for at least about 1 hour, 2 hours, 5 hours, 12 hours, or 24 hours. The temperature of the hybridization may be increased to adjust the stringency of the reaction, for example, from about 25° C. (room temperature), to about 45° C., 50° C., 55° C., 60° C., or 65° C. The hybridization reaction may also include another agent affecting the stringency, for example, hybridization conducted in the presence of 50% formamide increases the stringency of hybridization at a defined temperature.

The hybridization reaction may be followed by a single wash step, or two or more wash steps, which may be at the same or a different salinity and temperature. For example, the temperature of the wash may be increased to adjust the stringency from about 25° C. (room temperature), to about 45° C., 50° C., 55° C., 60° C., 65° C., or higher. The wash step may be conducted in the presence of a detergent, e.g., 0.1 or 0.2% SDS. For example, hybridization may be followed by two wash steps at 65° C. each for about 20 minutes in 2×SSC, 0.1% SDS, and optionally two additional wash steps at 65° C. each for about 20 minutes in 0.2×SSC, 0.1% SDS.

Exemplary stringent hybridization conditions include overnight hybridization at 65° C. in a solution comprising, or consisting of, 50% formamide, 10× Denhardt (0.2% Ficoll, 0.2% Polyvinylpyrrolidone, 0.2% bovine serum albumin) and 200 μg/ml of denatured carrier DNA, e.g., sheared salmon sperm DNA, followed by two wash steps at 65° C. each for about 20 minutes in 2×SSC, 0.1% SDS, and two wash steps at 65° C. each for about 20 minutes in 0.2×SSC, 0.1% SDS.

Hybridization may consist of hybridizing two nucleic acids in solution, or a nucleic acid in solution to a nucleic acid attached to a solid support, e.g., a filter. When one nucleic acid is on a solid support, a prehybridization step may be conducted prior to hybridization. Prehybridization may be carried out for at least about 1 hour, 3 hours or 10 hours in the same solution and at the same temperature as the hybridization solution (without the complementary polynucleotide strand).

Appropriate stringency conditions are known to those skilled in the art or may be determined experimentally by the skilled artisan. See, for example, Current Protocols in Molecular Biology, John Wiley & Sons, N.Y. (1989), 6.3.1-12.3.6; Sambrook et al., 1989, Molecular Cloning, A Laboratory Manual, Cold Spring Harbor Press, N.Y; S. Agrawal (ed.) Methods in Molecular Biology, volume 20; Tijssen (1993) Laboratory Techniques in biochemistry and molecular biology-hybridization with nucleic acid probes, e.g., part I chapter 2 “Overview of principles of hybridization and the strategy of nucleic acid probe assays”, Elsevier, New York; and Tibanyenda, N. et al., Eur. J. Biochem. 139:19 (1984) and Ebel, S. et al., Biochem. 31:12083 (1992).

The term “subject nucleic acid sequences” refers to all the nucleotide sequences that are subject nucleic acid sequences (predicted) and subject nucleic acid sequences (experimental). (as both those terms are defined below), and the term “a subject nucleic acid sequence” refers to one (and optionally more) of those nucleotide sequences. The term “subject nucleic acid sequences (experimental)” refers to the nucleotide sequences set forth in SEQ ID NO: 6, SEQ ID NO: 15, SEQ ID NO: 24, SEQ ID NO: 33, SEQ ID NO: 42, SEQ ID NO: 51, SEQ ID NO: 60, SEQ ID NO: 69, SEQ ID NO: 78, SEQ ID NO: 87, SEQ ID NO: 96, SEQ ID NO: 105, SEQ ID NO: 114, SEQ ID NO: 123, SEQ ID NO: 132, SEQ ID NO: 141, SEQ ID NO: 150, SEQ ID NO: 159, SEQ ID NO: 168, SEQ ID NO: 177, SEQ ID NO: 186, SEQ ID NO: 195, SEQ ID NO: 204, SEQ ID NO: 213, SEQ ID NO: 222, SEQ ID NO: 231, SEQ ID NO: 240, SEQ ID NO: 249, SEQ ID NO: 258, SEQ ID NO: 267, SEQ ID NO: 276, SEQ ID NO: 285, SEQ ID NO: 294, SEQ ID NO: 303, SEQ ID NO: 312, SEQ ID NO: 321, SEQ ID NO: 330, SEQ ID NO: 339, SEQ ID NO: 348, SEQ ID NO: 357, and any other nucleic acid sequences set forth in the Figures that by comparison to the foregoing sequences should be included in this definition, and the term “a subject nucleic acid sequence (experimental)” refers to one (and optionally more) of those nucleotide sequences. The term “subject nucleic acid sequences (predicted)” refers to the nucleotide sequences set forth in SEQ ID NO: 4, SEQ ID NO: 13, SEQ ID NO: 22, SEQ ID NO: 31, SEQ ID NO: 40, SEQ ID NO: 49, SEQ ID NO: 58, SEQ ID NO: 67, SEQ ID NO: 76, SEQ ID NO: 85, SEQ ID NO: 94, SEQ ID NO: 103, SEQ ID NO: 112, SEQ ID NO: 121, SEQ ID NO: 130, SEQ ID NO: 139, SEQ ID NO: 148, SEQ ID NO: 157, SEQ ID NO: 166, SEQ ID NO: 175, SEQ ID NO: 184, SEQ ID NO: 193, SEQ ID NO: 202, SEQ ID NO: 211, SEQ ID NO: 220, SEQ ID NO: 229, SEQ ID NO: 238, SEQ ID NO: 247, SEQ ID NO: 256, SEQ ID NO: 265, SEQ ID NO: 274, SEQ ID NO: 283, SEQ ID NO: 292, SEQ ID NO: 301, SEQ ID NO: 310, SEQ ID NO: 319, SEQ ID NO: 328, SEQ ID NO: 337, SEQ ID NO: 346, SEQ ID NO: 355, and any other nucleic acid sequences set forth in the Figures that by comparison to the foregoing sequences should be included in this definition, and the term “a subject nucleic acid sequence (predicted)” refers to one (and optionally more) of those nucleotide sequences.

The term “subject amino acid sequences” refers to all the amino acid sequences that are subject amino acid sequences (predicted) and subject amino acid sequences (experimental) (as both those terms are defined below), and the term “a subject amino acid sequence” refers to one (and optionally more) of those amino acid sequences. The term “subject amino acid sequences (experimental)” refers to the amino acid sequences set forth in SEQ ID NO: 7, SEQ ID NO: 16, SEQ ID NO: 25, SEQ ID NO: 34, SEQ ID NO: 43, SEQ ID NO: 52, SEQ ID NO: 61, SEQ ID NO: 70, SEQ ID NO: 79, SEQ ID NO: 88, SEQ ID NO: 97, SEQ ID NO: 106, SEQ ID NO: 115, SEQ ID NO: 124, SEQ ID NO: 133, SEQ ID NO: 142, SEQ ID NO: 151, SEQ ID NO: 160, SEQ ID NO: 169, SEQ ID NO: 178, SEQ ID NO: 187, SEQ ID NO: 196, SEQ ID NO: 205, SEQ ID NO: 214, SEQ ID NO: 223, SEQ ID NO: 232, SEQ ID NO: 241, SEQ ID NO: 250, SEQ ID NO: 259, SEQ ID NO: 268, SEQ ID NO: 277, SEQ ID NO: 286, SEQ ID NO: 295, SEQ ID NO: 304, SEQ ID NO: 313, SEQ ID NO: 322, SEQ ID NO: 331, SEQ ID NO: 340, SEQ ID NO: 349, SEQ ID NO: 358, and any other amino acid sequences set forth in the Figures that by comparison to the foregoing sequences should be included in this definition, and the term “a subject amino acid sequence (experimental)” refers to one (and optionally more) of those amino acid sequences. The term “subject amino acid sequences (predicted)” refers to the amino acid sequences set forth in SEQ ID NO: 5, SEQ ID NO: 14, SEQ ID NO: 23, SEQ ID NO: 32, SEQ ID NO: 41, SEQ ID NO: 50, SEQ ID NO: 59, SEQ ID NO: 68, SEQ ID NO: 77, SEQ ID NO: 86, SEQ ID NO: 95, SEQ ID NO: 104, SEQ ID NO: 113, SEQ ID NO: 122, SEQ ID NO: 131, SEQ ID NO: 140, SEQ ID NO: 149, SEQ ID NO: 158, SEQ ID NO: 167, SEQ ID NO: 176, SEQ ID NO: 185, SEQ ID NO: 194, SEQ ID NO: 203, SEQ ID NO: 212, SEQ ID NO: 221, SEQ ID NO: 230, SEQ ID NO: 239, SEQ ID NO: 248, SEQ ID NO: 257, SEQ ID NO: 266, SEQ ID NO: 275, SEQ ID NO: 284, SEQ ID NO: 293, SEQ ID NO: 302, SEQ ID NO: 311, SEQ ID NO: 320, SEQ ID NO: 329, SEQ ID NO: 338, SEQ ID NO: 347, SEQ ID NO: 356, and any other amino acid sequences set forth in the Figures that by comparison to the foregoing sequences should be included in this definition, and the term “a subject amino acid sequence (predicted)” refers to one (and optionally more) of those amino acid sequences.

As applied to proteins, the term “substantial identity” means that two protein sequences, when optimally aligned, such as by the programs GAP or BESTFIT using default gap weights, typically share at least about 70 percent sequence identity, alternatively at least about 80, 85, 90, 95 percent sequence identity or more. In certain instances, residue positions that are not identical differ by conservative amino acid substitutions, which are described above.

The term “structural motif”, when used in reference to a polypeptide, refers to a polypeptide that, although it may have different amino acid sequences, may result in a similar structure, wherein by structure is meant that the motif forms generally the same tertiary structure, or that certain amino acid residues within the motif, or alternatively their backbone or side chains (which may or may not include the Cα atoms of the side chains) are positioned in a like relationship with respect to one another in the motif.

The term “test compound” refers to a molecule to be tested by one or more screening method(s) as a putative modulator of a polypeptide of the invention or other biological entity or process. A test compound is usually not known to bind to a target of interest. The term “control test compound” refers to a compound known to bind to the target (e.g., a known agonist, antagonist, partial agonist or inverse agonist). The term “test compound” does not include a chemical added as a control condition that alters the function of the target to determine signal specificity in an assay. Such control chemicals or conditions include chemicals that 1) nonspecifically or substantially disrupt protein structure (e.g., denaturing agents (e.g., urea or guanidinium), chaotropic agents, sulfhydryl reagents (e.g., dithiothreitol and β-mercaptoethanol), and proteases), 2) generally inhibit cell metabolism (e.g., mitochondrial uncouplers) and 3) non-specifically disrupt electrostatic or hydrophobic interactions of a protein (e.g., high salt concentrations, or detergents at concentrations sufficient to non-specifically disrupt hydrophobic interactions). Further, the term “test compound” also does not include compounds known to be unsuitable for a therapeutic use for a particular indication due to toxicity of the subject. In certain embodiments, various predetermined concentrations of test compounds are used for screening such as 0.01 μM, 0.1 μM, 1.0 μM, and 10.0 μM. Examples of test compounds include, but are not limited to, peptides, nucleic acids, carbohydrates, and small molecules. The term “novel test compound” refers to a test compound that is not in existence as of the filing date of this application. In certain assays using novel test compounds, the novel test compounds comprise at least about 50%, 75%, 85%, 90%, 95% or more of the test compounds used in the assay or in any particular trial of the assay.

The term “therapeutically effective amount” refers to that amount of a modulator, drug or other molecule which is sufficient to effect treatment when administered to a subject in need of such treatment. The therapeutically effective amount will vary depending upon the subject and disease condition being treated, the weight and age of the subject, the severity of the disease condition, the manner of administration and the like, which can readily be determined by one of ordinary skill in the art.

The term “transfection” means the introduction of a nucleic acid, e.g., an expression vector, into a recipient cell, which in certain instances involves nucleic acid-mediated gene transfer. The term “transformation” refers to a process in which a cell's genotype is changed as a result of the cellular uptake of exogenous nucleic acid. For example, a transformed cell may express a recombinant form of a polypeptide of the invention or antisense expression may occur from the transferred gene so that the expression of a naturally-occurring form of the gene is disrupted.

The term “transgene” means a nucleic acid sequence, which is partly or entirely heterologous to a transgenic animal or cell into which it is introduced, or, is homologous to an endogenous gene of the transgenic animal or cell into which it is introduced, but which is designed to be inserted, or is inserted, into the animal's genome in such a way as to alter the genome of the cell into which it is inserted (e.g., it is inserted at a location which differs from that of the natural gene or its insertion results in a knockout). A transgene may include one or more regulatory sequences and any other nucleic acids, such as introns, that may be necessary for optimal expression.

The term “transgenic animal” refers to any animal, for example, a mouse, rat or other non-human mammal, a bird or an amphibian, in which one or more of the cells of the animal contain heterologous nucleic acid introduced by way of human intervention, such as by transgenic techniques well known in the art. The nucleic acid is introduced into the cell, directly or indirectly, by way of deliberate genetic manipulation, such as by microinjection or by infection with a recombinant virus. The term genetic manipulation does not include classical cross-breeding, or in vitro fertilization, but rather is directed to the introduction of a recombinant DNA molecule. This molecule may be integrated within a chromosome, or it may be extrachromosomally replicating DNA. In the typical transgenic animals described herein, the transgene causes cells to express a recombinant form of a protein. However, transgenic animals in which the recombinant gene is silent are also contemplated.

The term “vector” refers to a nucleic acid capable of transporting another nucleic acid to which it has been linked. One type of vector which may be used in accord with the invention is an episome, i.e., a nucleic acid capable of extra-chromosomal replication. Other vectors include those capable of autonomous replication and expression of nucleic acids to which they are linked. Vectors capable of directing the expression of genes to which they are operatively linked are referred to herein as “expression vectors”. In general, expression vectors of utility in recombinant DNA techniques are often in the form of “plasmids” which refer to circular double stranded DNA molecules which, in their vector form are not bound to the chromosome. In the present specification, “plasmid” and “vector” are used interchangeably as the plasmid is the most commonly used form of vector. However, the invention is intended to include such other forms of expression vectors which serve equivalent functions and which become known in the art subsequently hereto.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that may vary depending upon the desired properties sought to be obtained by the present invention.

2. Polypeptides of the Invention

The present invention makes available in a variety of embodiments soluble, purified and/or isolated forms of the polypeptides of the invention. Milligram quantities of exemplary polypeptides of the invention (optionally with a tag and optionally labeled) have been isolated in a highly purified form. The present invention provides for expressing and purifying polypeptides of the invention in quantities that equal or exceed the quantity of polypeptide(s) of the invention expressed and purified as provided in the Exemplification section below (or smaller amount(s) thereof, such as 25%, 33%, 50% or 75% of the amount(s) so expressed and/or purified).

In one aspect, the present invention contemplates an isolated polypeptide comprising (a) a subject amino acid sequence, (b) the subject amino acid sequence with 1 to about 20 conservative amino acid substitutions, deletions or additions, (c) an amino acid sequence that is at least 90% identical to the subject amino acid sequence, or (d) a functional fragment of a polypeptide having an amino acid sequence set forth in (a), (b) or (c). In another aspect, the present invention contemplates a composition comprising such an isolated polypeptide and less than about 10%, or alternatively 5%, or alternatively 1%, contaminating biological macromolecules or polypeptides.

It may be the case that the amino acid sequence for a polypeptide of the invention predicted from the publicly available genomic information differs from the amino acid sequence determined from the experimentally determined nucleic acid by one or more amino acids. For example, in the case of GTP-binding protein (era) from P. aeruginosa, SEQ ID NO: 7 is determined from the experimentally determined nucleic acid sequence SEQ ID NO: 6, and SEQ ID NO: 5 is determined from SEQ ID NO: 4, which is obtained as described in EXAMPLE 1. In such a case, the present invention contemplates the specific amino acid sequences of SEQ ID NO: 5 and SEQ ID NO: 7, and variants thereof, as well as any differences (if any) in the polypeptides of the invention based on those SEQ ID NOS and nucleic acid sequences encoding the same (including subject nucleic acid sequences).

In certain embodiments, a polypeptide of the invention is a fusion protein containing a domain which increases its solubility and/or facilitates its purification, identification, detection, and/or structural characterization. Exemplary domains, include, for example, glutathione S-transferase (GST), protein A, protein G, calmodulin-binding peptide, thioredoxin, maltose binding protein, HA, myc, poly arginine, poly His, poly His-Asp or FLAG fusion proteins and tags. Additional exemplary domains include domains that alter protein localization in vivo, such as signal peptides, type III secretion system-targeting peptides, transcytosis domains, nuclear localization signals, etc. In various embodiments, a polypeptide of the invention may comprise one or more heterologous fusions. Polypeptides may contain multiple copies of the same fusion domain or may contain fusions to two or more different domains. The fusions may occur at the N-terminus of the polypeptide, at the C-terminus of the polypeptide, or at both the N- and C-terminus of the polypeptide. It is also within the scope of the invention to include linker sequences between a polypeptide of the invention and the fusion domain in order to facilitate construction of the fusion protein or to optimize protein expression or structural constraints of the fusion protein. In another embodiment, the polypeptide may be constructed so as to contain protease cleavage sites between the fusion polypeptide and polypeptide of the invention in order to remove the tag after protein expression or thereafter. Examples of suitable endoproteases, include, for example, Factor Xa and TEV proteases.

In another embodiment, a polypeptide of the invention may be modified so that its rate of traversing the cellular membrane is increased. For example, the polypeptide may be fused to a second peptide which promotes “tanscytosis,” e.g., uptake of the peptide by cells. The peptide may be a portion of the HIV transactivator (TAT) protein, such as the fragment corresponding to residues 37-62 or 48-60 of TAT, portions which have been observed to be rapidly taken up by a cell in vitro (Green and Loewenstein, (1989) Cell 55:1179-1188). Alternatively, the internalizing peptide may be derived from the Drosophila antennapedia protein, or homologs thereof. The 60 amino acid long homeodomain of the homeo-protein antennapedia has been demonstrated to translocate through biological membranes and can facilitate the translocation of heterologous polypeptides to which it is coupled. Thus, polypeptides may be fused to a peptide consisting of about amino acids 42-58 of Drosophila antennapedia or shorter fragments for transcytosis. (Derossi et al. (1996) J Biol Chem 271:18188-18193; Derossi et al. (1994) J Biol Chem 269:10444-10450; and Perez et al. (1992) J Cell Sci 102:717-722). The transcytosis polypeptide may also be a non-naturally-occurring membrane-translocating sequence (MTS), such as the peptide sequences disclosed in U.S. Pat. No. 6,248,558.

In another embodiment, a polypeptide of the invention is labeled with an isotopic label to facilitate its detection and or structural characterization using nuclear magnetic resonance or another applicable technique. Exemplary isotopic labels include radioisotopic labels such as, for example, potassium-40 (⁴⁰K), carbon-14 (¹⁴C), tritium (³H), sulphur-35 (³⁵S), phosphorus-32 (³²P), technetium-99m (^{99m}Tc), thallium-201 (²⁰¹Tl), gallium-67 (⁶⁷Ga), indium-111 (¹¹¹In), iodine-123 (¹²³I), iodine-131 (¹³¹I), yttrium-90 (⁹⁰Y), samarium-153 (¹⁵³Sm), rhenium-186 (¹⁸⁶Re), rhenium-188 (¹⁸⁸Re), dysprosium-165 (¹⁶⁵Dy) and holmium-166 (¹⁶⁶Ho). The isotopic label may also be an atom with non zero nuclear spin, including, for example, hydrogen-1 (¹H), hydrogen-2 (²H), hydrogen-3 (³H), phosphorous-31 (³¹P), sodium-23 (²³Na), nitrogen-14 (¹⁴N), nitrogen-15 (¹⁵N), carbon-13 (¹³C) and fluorine-19 (¹⁹F). In certain embodiments, the polypeptide is uniformly labeled with an isotopic label, for example, wherein at least 50%, 70%, 80%, 90%, 95%, or 98% of the possible labels in the polypeptide are labeled, e.g., wherein at least 50%, 70%, 80%, 90%, 95%, or 98% of the nitrogen atoms in the polypeptide are ¹⁵N, and/or wherein at least 50%, 70%, 80%, 90%, 95%, or 98% of the carbon atoms in the polypeptide are ¹³C, and/or wherein at least 50%, 70%, 80%, 90%, 95%, or 98% of the hydrogen atoms in the polypeptide are ²H. In other embodiments, the isotopic label is located in one or more specific locations within the polypeptide, for example, the label may be specifically incorporated into one or more of the leucine residues of the polypeptide. The invention also encompasses the embodiment wherein a single polypeptide comprises two, three or more different isotopic labels, for example, the polypeptide comprises both ¹⁵N and ¹³C labeling.

In yet another embodiment, the polypeptides of the invention are labeled to facilitate structural characterization using x-ray crystallography or another applicable technique. Exemplary labels include heavy atom labels such as, for example, cobalt, selenium, krypton, bromine, strontium, molybdenum, ruthenium, rhodium, palladium, silver, cadmium, tin, iodine, xenon, barium, lanthanum, cerium, praseodymium, neodymium, samarium, europium, gadolinium, terbium, dysprosium, holmium, erbium, thulium, ytterbium, lutetium, tantalum, tungsten, rhenium, osmium, iridium, platinum, gold, mercury, thallium, lead, thorium and uranium. In an exemplary embodiment, the polypeptide is labeled with seleno-methionine.

A variety of methods are available for preparing a polypeptide with a label, such as a radioisotopic label or heavy atom label. For example, in one such method, an expression vector comprising a nucleic acid encoding a polypeptide is introduced into a host cell, and the host cell is cultured in a cell culture medium in the presence of a source of the label, thereby generating a labeled polypeptide. As indicated above, the extent to which a polypeptide may be labeled may vary.

In still another embodiment, the polypeptides of the invention are labeled with a fluorescent label to facilitate their detection, purification, or structural characterization. In an exemplary embodiment, a polypeptide of the invention is fused to a heterologous polypeptide sequence which produces a detectable fluorescent signal, including, for example, green fluorescent protein (GFP), enhanced green fluorescent protein (EGFP), Renilla Reniformis green fluorescent protein, GFPmut2, GFPuv4, enhanced yellow fluorescent protein (EYFP), enhanced cyan fluorescent protein (ECFP), enhanced blue fluorescent protein (EBFP), citrine and red fluorescent protein from discosoma (dsRED).

In other embodiments, the invention provides for polypeptides of the invention immobilized onto a solid surface, including, plates, microtiter plates, slides, beads, particles, spheres, films, strands, precipitates, gels, sheets, tubing, containers, capillaries, pads, slices, etc. The polypeptides of the invention may be immobilized onto a “chip” as part of an array. An array, having a plurality of addresses, may comprise one or more polypeptides of the invention in one or more of those addresses. In one embodiment, the chip comprises one or more polypeptides of the invention as part of an array that contains at least some polypeptide sequences from the pathogen of origin.

In still other embodiments, the invention comprises the polypeptide sequences of the invention in computer readable format. The invention also encompasses a database comprising the polypeptide sequences of the invention.

In other embodiments, the invention relates to the polypeptides of the invention contained within a vessels useful for manipulation of the polypeptide sample. For example, the polypeptides of the invention may be contained within a microtiter plate to facilitate detection, screening or purification of the polypeptide. The polypeptides may also be contained within a syringe as a container suitable for administering the polypeptide to a subject in order to generate antibodies or as part of a vaccination regimen. The polypeptides may also be contained within an NMR tube in order to enable characterization by nuclear magnetic resonance techniques.

In still other embodiments, the invention relates to a crystallized polypeptide of the invention and crystallized polypeptides which have been mounted for examination by x-ray crystallography as described further below. In certain instances, a polypeptide of the invention in crystal form may be single crystals of various dimensions (e.g., micro-crystals) or may be an aggregate of crystalline material. In another aspect, the present invention contemplates a crystallized complex including a polypeptide of the invention and one or more of the following: a co-factor (such as a salt, metal, nucleotide, oligonucleotide or polypeptide), a modulator, or a small molecule. In another aspect, the present invention contemplates a crystallized complex including a polypeptide of the invention and any other molecule or atom (such as a metal ion) that associates with the polypeptide in vivo.

In certain embodiments, polypeptides of the invention may be synthesized chemically, ribosomally in a cell free system, or ribosomally within a cell. Chemical synthesis of polypeptides of the invention may be carried out using a variety of art recognized methods, including stepwise solid phase synthesis, semi-synthesis through the conformationally-assisted re-ligation of peptide fragments, enzymatic ligation of cloned or synthetic peptide segments, and chemical ligation. Native chemical ligation employs a chemoselective reaction of two unprotected peptide segments to produce a transient thioester-linked intermediate. The transient thioester-linked intermediate then spontaneously undergoes a rearrangement to provide the full length ligation product having a native peptide bond at the ligation site. Full length ligation products are chemically identical to proteins produced by cell free synthesis. Full length ligation products may be refolded and/or oxidized, as allowed, to form native disulfide-containing protein molecules. (see e.g., U.S. Pat. Nos. 6,184,344 and 6,174,530; and T. W. Muir et al., Curr. Opin. Biotech. (1993): vol. 4, p 420; M. Miller, et al., Science (1989): vol. 246, p 1149; A. Wlodawer, et al., Science (1989): vol. 245, p 616; L. H. Huang, et al., Biochemistry (1991): vol. 30, p 7402; M. Schnolzer, et al., Int. J. Pept. Prot. Res. (1992): vol. 40, p 180-193; K. Rajarathnam, et al., Science (1994): vol. 264, p 90; R. E. Offord, “Chemical Approaches to Protein Engineering”, in Protein Design and the Development of New therapeutics and Vaccines, J. B. Hook, G. Poste, Eds., (Plenum Press, New York, 1990) pp. 253-282; C. J. A. Wallace, et al., J. Biol. Chem. (1992): vol. 267, p 3852; L. Abrahmsen, et al., Biochemistry (1991): vol. 30, p 4151; T. K. Chang, et al., Proc. Natl. Acad. Sci. USA (1994) 91: 12544-12548; M. Schnlzer, et al., Science (1992): vol., 3256, p 221; and K. Akaji, et al., Chem. Pharm. Bull. (Tokyo) (1985) 33: 184).

In certain embodiments, it may be advantageous to provide naturally-occurring or experimentally-derived homologs of a polypeptide of the invention. Such homologs may function in a limited capacity as a modulator to promote or inhibit a subset of the biological activities of the naturally-occurring form of the polypeptide. Thus, specific biological effects may be elicited by treatment with a homolog of limited function, and with fewer side effects relative to treatment with agonists or antagonists which are directed to all of the biological activities of a polypeptide of the invention. For instance, antagonistic homologs may be generated which interfere with the ability of the wild-type polypeptide of the invention to associate with certain proteins, but which do not substantially interfere with the formation of complexes between the native polypeptide and other cellular proteins. Another aspect of the invention relates to polypeptides derived from the full-length polypeptides of the invention. Isolated peptidyl portions of those polypeptides may be obtained by screening polypeptides recombinantly produced from the corresponding fragment of the nucleic acid encoding such polypeptides. In addition, fragments may be chemically synthesized using techniques known in the art such as conventional Merrifield solid phase f-Moc or t-Boc chemistry. For example, proteins may be arbitrarily divided into fragments of desired length with no overlap of the fragments, or may be divided into overlapping fragments of a desired length. The fragments may be produced (recombinantly or by chemical synthesis) and tested to identify those peptidyl fragments having a desired property, for example, the capability of functioning as a modulator of the polypeptides of the invention. In an illustrative embodiment, peptidyl portions of a protein of the invention may be tested for binding activity, as well as inhibitory ability, by expression as, for example, thioredoxin fusion proteins, each of which contains a discrete fragment of a protein of the invention (see, for example, U.S. Pat. Nos. 5,270,181 and 5,292,646; and PCT publication WO94/02502).

In another embodiment, truncated polypeptides may be prepared. Truncated polypeptides have from 1 to 20 or more amino acid residues removed from either or both the N- and C-termini. Such truncated polypeptides may prove more amenable to expression, purification or characterization than the full-length polypeptide. For example, truncated polypeptides may prove more amenable than the full-length polypeptide to crystallization, to yielding high quality diffracting crystals or to yielding an HSQC with high intensity peaks and minimally overlapping peaks. In addition, the use of truncated polypeptides may also identify stable and active domains of the full-length polypeptide that may be more amenable to characterization.

It is also possible to modify the structure of the polypeptides of the invention for such purposes as enhancing therapeutic or prophylactic efficacy, or stability (e.g., ex vivo shelf life, resistance to proteolytic degradation in vivo, etc.). Such modified polypeptides, when designed to retain at least one activity of the naturally-occurring form of the protein, are considered “functional equivalents” of the polypeptides described in more detail herein. Such modified polypeptides may be produced, for instance, by amino acid substitution, deletion, or addition, which substitutions may consist in whole or part by conservative amino acid substitutions.

For instance, it is reasonable to expect that an isolated conservative amino acid substitution, such as replacement of a leucine with an isoleucine or valine, an aspartate with a glutamate, a threonine with a serine, will not have a major affect on the biological activity of the resulting molecule. Whether a change in the amino acid sequence of a polypeptide results in a functional homolog may be readily determined by assessing the ability of the variant polypeptide to produce a response similar to that of the wild-type protein. Polypeptides in which more than one replacement has taken place may readily be tested in the same manner.

This invention further contemplates a method of generating sets of combinatorial mutants of polypeptides of the invention, as well as truncation mutants, and is especially useful for identifying potential variant sequences (e.g. homologs). The purpose of screening such combinatorial libraries is to generate, for example, homologs which may modulate the activity of a polypeptide of the invention, or alternatively, which possess novel activities altogether. Combinatorially-derived homologs may be generated which have a selective potency relative to a naturally-occurring protein. Such homologs may be used in the development of therapeutics.

Likewise, mutagenesis may give rise to homologs which have intracellular half-lives dramatically different than the corresponding wild-type protein. For example, the altered protein may be rendered either more stable or less stable to proteolytic degradation or other cellular process which result in destruction of, or otherwise inactivation of the protein. Such homologs, and the genes which encode them, may be utilized to alter protein expression by modulating the half-life of the protein. As above, such proteins may be used for the development of therapeutics or treatment.

In similar fashion, protein homologs may be generated by the present combinatorial approach to act as antagonists, in that they are able to interfere with the activity of the corresponding wild-type protein.

In a representative embodiment of this method, the amino acid sequences for a population of protein homologs are aligned, preferably to promote the highest homology possible. Such a population of variants may include, for example, homologs from one or more species, or homologs from the same species but which differ due to mutation. Amino acids which appear at each position of the aligned sequences are selected to create a degenerate set of combinatorial sequences. In certain embodiments, the combinatorial library is produced by way of a degenerate library of genes encoding a library of polypeptides which each include at least a portion of potential protein sequences. For instance, a mixture of synthetic oligonucleotides may be enzymatically ligated into gene sequences such that the degenerate set of potential nucleotide sequences are expressible as individual polypeptides, or alternatively, as a set of larger fusion proteins (e.g. for phage display).

There are many ways by which the library of potential homologs may be generated from a degenerate oligonucleotide sequence. Chemical synthesis of a degenerate gene sequence may be carried out in an automatic DNA synthesizer, and the synthetic genes may then be ligated into an appropriate vector for expression. One purpose of a degenerate set of genes is to provide, in one mixture, all of the sequences encoding the desired set of potential protein sequences. The synthesis of degenerate oligonucleotides is well known in the art (see for example, Narang, S A (1983) Tetrahedron 39:3; Itakura et al., (1981) Recombinant DNA, Proc. 3rd Cleveland Sympos. Macromolecules, ed. A G Walton, Amsterdam: Elsevier pp. 273-289; Itakura et al., (1984) Annu. Rev. Biochem. 53:323; Itakura et al., (1984) Science 198:1056; Ike et al., (1983) Nucleic Acid Res. 11:477). Such techniques have been employed in the directed evolution of other proteins (see, for example, Scott et al., (1990) Science 249:386-390; Roberts et al., (1992) PNAS USA 89:2429-2433; Devlin et al., (1990) Science 249: 404-406; Cwirla et al., (1990) PNAS USA 87: 6378-6382; as well as U.S. Pat. Nos. 5,223,409, 5,198,346, and 5,096,815).

Alternatively, other forms of mutagenesis may be utilized to generate a combinatorial library. For example, protein homologs (both agonist and antagonist forms) may be generated and isolated from a library by screening using, for example, alanine scanning mutagenesis and the like (Ruf et al., (1994) Biochemistry 33:1565-1572; Wang et al., (1994) J. Biol. Chem. 269:3095-3099; Balint et al., (1993) Gene 137:109-118; Grodberg et al., (1993) Eur. J. Biochem. 218:597-601; Nagashima et al., (1993) J. Biol. Chem. 268:2888-2892; Lowman et al., (1991) Biochemistry 30:10832-10838; and Cunningham et al., (1989) Science 244:1081-1085), by linker scanning mutagenesis (Gustin et al., (1993) Virology 193:653-660; Brown et al., (1992) Mol. Cell Biol. 12:2644-2652; McKnight et al., (1982) Science 232:316); by saturation mutagenesis (Meyers et al., (1986) Science 232:613); by PCR mutagenesis (Leung et al., (1989) Method Cell Mol Biol 1: 11-19); or by random mutagenesis (Miller et al., (1992) A Short Course in Bacterial Genetics, CSHL Press, Cold Spring Harbor, N.Y.; and Greener et al., (1994) Strategies in Mol Biol 7:32-34). Linker scanning mutagenesis, particularly in a combinatorial setting, is an attractive method for identifying truncated forms of proteins that are bioactive.

A wide range of techniques are known in the art for screening gene products of combinatorial libraries made by point mutations and truncations, and for screening cDNA libraries for gene products having a certain property. Such techniques will be generally adaptable for rapid screening of the gene libraries generated by the combinatorial mutagenesis of protein homologs. The most widely used techniques for screening large gene libraries typically comprises cloning the gene library into replicable expression vectors, transforming appropriate cells with the resulting library of vectors, and expressing the combinatorial genes under conditions in which detection of a desired activity facilitates relatively easy isolation of the vector encoding the gene whose product was detected. Each of the illustrative assays described below are amenable to high throughput analysis as necessary to screen large numbers of degenerate sequences created by combinatorial mutagenesis techniques.

In an illustrative embodiment of a screening assay, candidate combinatorial gene products are displayed on the surface of a cell and the ability of particular cells or viral particles to bind to the combinatorial gene product is detected in a “panning assay”. For instance, the gene library may be cloned into the gene for a surface membrane protein of a bacterial cell (Ladner et al., WO 88/06630; Fuchs et al., (1991) Bio/Technology 9:1370-1371; and Goward et al., (1992) TIBS 18:136-140), and the resulting fusion protein detected by panning, e.g. using a fluorescently labeled molecule which binds the cell surface protein, e.g. FITC-substrate, to score for potentially functional homologs. Cells may be visually inspected and separated under a fluorescence microscope, or, when the morphology of the cell permits, separated by a fluorescence-activated cell sorter. This method may be used to identity substrates or other polypeptides that can interact with a polypeptide of the invention.

In similar fashion, the gene library may be expressed as a fusion protein on the surface of a viral particle. For instance, in the filamentous phage system, foreign peptide sequences may be expressed on the surface of infectious phage, thereby conferring two benefits. First, because these phage may be applied to affinity matrices at very high concentrations, a large number of phage may be screened at one time. Second, because each infectious phage displays the combinatorial gene product on its surface, if a particular phage is recovered from an affinity matrix in low yield, the phage may be amplified by another round of infection. The group of almost identical E. coli filamentous phages M13, fd, and fl are most often used in phage display libraries, as either of the phage gIII or gVIII coat proteins may be used to generate fusion proteins without disrupting the ultimate packaging of the viral particle (Ladner et al., PCT publication WO 90/02909; Garrard et al., PCT publication WO 92/09690; Marks et al., (1992) J. Biol. Chem. 267:16007-16010; Griffiths et al., (1993) EMBO J. 12:725-734; Clackson et al., (1991) Nature 352:624-628; and Barbas et al., (1992) PNAS USA 89:4457-4461). Other phage coat proteins may be used as appropriate.

The invention also provides for reduction of the polypeptides of the invention to generate mimetics, e.g. peptide or non-peptide agents, which are able to mimic binding of the authentic protein to another cellular partner. Such mutagenic techniques as described above, as well as the thioredoxin system, are also particularly useful for mapping the determinants of a protein which participates in a protein-protein interaction with another protein. To illustrate, the critical residues of a protein which are involved in molecular recognition of a substrate protein may be determined and used to generate peptidomimetics that may bind to the substrate protein. The peptidomimetic may then be used as an inhibitor of the wild-type protein by binding to the substrate and covering up the critical residues needed for interaction with the wild-type protein, thereby preventing interaction of the protein and the substrate. By employing, for example, scanning mutagenesis to map the amino acid residues of a protein which are involved in binding a substrate polypeptide, peptidomimetic compounds may be generated which mimic those residues in binding to the substrate. For instance, non-hydrolyzable peptide analogs of such residues may be generated using benzodiazepine (e.g., see Freidinger et al., in Peptides: Chemistry and Biology, G. R. Marshall ed., ESCOM Publisher: Leiden, Netherlands, 1988), azepine (e.g., see Huffman et al., in Peptides: Chemistry and Biology, G. R. Marshall ed., ESCOM Publisher: Leiden, Netherlands, 1988), substituted gamma lactam rings (Garvey et al., in Peptides: Chemistry and Biology, G. R. Marshall ed., ESCOM Publisher: Leiden, Netherlands, 1988), keto-methylene pseudopeptides (Ewenson et al., (1986) J. Med. Chem. 29:295; and Ewenson et al., in Peptides: Structure and Function (Proceedings of the 9th American Peptide Symposium) Pierce Chemical Co. Rockland, Ill., 1985), β-turn dipeptide cores (Nagai et al., (1985) Tetrahedron Lett 26:647; and Sato et al., (1986) J Chem Soc Perkin Trans 1:1231), and β-aminoalcohols (Gordon et al., (1985) Biochem Biophys Res Commun 126:419; and Dann et al., (1986) Biochem Biophys Res Commun 134:71).

The activity of a polypeptide of the invention may be identified and/or assayed using a variety of methods well known to the skilled artisan. For example, information about the activity of non-essential genes may be assayed by creating a null mutant strain of bacteria expressing a mutant form of, or lacking expression of, a protein of interest. The resulting phenotype of the null mutant strain may provide information about the activity of the mutated gene product. Essential genes may be studied by creating a bacterial strain with a conditional mutation in the gene of interest. The bacterial strain may be grown under permissive and non-permissive conditions and the change in phenotype under the non-permissive conditions may be used to identify and/or assay the activity of the gene product.

In an alternative embodiment, the activity of a protein may be assayed using an appropriate substrate or binding partner or other reagent suitable to test for the suspected activity. For catalytic activity, the assay is typically designed so that the enzymatic reaction produces a detectable signal. For example, mixture of a kinase with a substrate in the presence of ³²P will result in incorporation of the ³²P into the substrate. The labeled substrate may then be separated from the free ³²P and the presence and/or amount of radiolabeled substrate may be detected using a scintillation counter or a phosphorimager. Similar assays may be designed to identify and/or assay the activity of a wide variety of enzymatic activities. Based on the teachings herein, the skilled artisan would readily be able to develop an appropriate assay for a polypeptide of the invention.

In another embodiment, the activity of a polypeptide of the invention may be determined by assaying for the level of expression of RNA and/or protein molecules. Transcription levels may be determined, for example, using Northern blots, hybridization to an oligonucleotide array or by assaying for the level of a resulting protein product. Translation levels may be determined, for example, using Western blotting or by identifying a detectable signal produced by a protein product (e.g., fluorescence, luminescence, enzymatic activity, etc.). Depending on the particular situation, it may be desirable to detect the level of transcription and/or translation of a single gene or of multiple genes.

Alternatively, it may be desirable to measure the overall rate of DNA replication, transcription and/or translation in a cell. In general this may be accomplished by growing the cell in the presence of a detectable metabolite which is incorporated into the resultant DNA, RNA, or protein product. For example, the rate of DNA synthesis may be determined by growing cells in the presence of BrdU which is incorporated into the newly synthesized DNA. The amount of BrdU may then be determined histochemically using an anti-BrdU antibody.

In general, the polypeptides of the invention are expected to be involved in bacterial viability. The expected biological activity of certain of the polypeptides of the invention is indicated in the following table, as described in further detail below.

| | Bacterial | Protein | Gene | COG | COG ID |
|---|---|---|---|---|---|
| SEQ ID NOS | Species | Annotation | Designation | Category | Number |
| SEQ ID NO: 5 | P. aeruginosa | GTP-binding | era | General | COG1159 |
| SEQ ID NO: 7 | | protein Era | | function | |
| | | | | prediction | |
| SEQ ID NO: 14 | E. faecalis | short chain | scd | Not available | N/A |
| SEQ ID NO: 16 | | dehydrogenase | | | |
| | | family protein | | | |
| SEQ ID NO: 23 | E. faecalis | glucose-inhibited | gidB | Not available | N/A |
| SEQ ID NO: 25 | | division protein B | | | |
| SEQ ID NO: 32 | E. faecalis | N utilization | nusB | Not available | N/A |
| SEQ ID NO: 34 | | substance protein B | | | |
| SEQ ID NO: 41 | E. faecalis | translation | tufA | Not available | N/A |
| SEQ ID NO: 43 | | elongation factor | | | |
| | | Tu | | | |
| SEQ ID NO: 50 | E. faecalis | GTP-binding | yhbZ | Not available | N/A |
| SEQ ID NO: 52 | | protein | (obg) | | |
| SEQ ID NO: 59 | S. pneumoniae | shikimate 5- | aroE | Amino acid | COG0169 |
| SEQ ID NO: 61 | | dehydrogenase | | transport and | |
| | | | | metabolism | |
| SEQ ID NO: 68 | P. aeruginosa | conserved | b1983 | Unknown | COG0217 |
| SEQ ID NO: 70 | | hypothetical | | function | |
| | | protein | | | |
| SEQ ID NO: 77 | P. aeruginosa | beta-ketoacyl- | fabB | Lipid | COG0304 |
| SEQ ID NO: 79 | | ACP synthase I | | metabolism | |
| SEQ ID NO: 86 | P. aeruginosa | ribosome | frr | Translation, | COG0233 |
| SEQ ID NO: 88 | | recycling factor | | ribosomal | |
| | | | | structure, and | |
| | | | | biogenesis | |
| SEQ ID NO: 95 | S. pneumoniae | N utilization | nusA | Transcription | COG0195 |
| SEQ ID NO: 97 | | substance protein A | | | |
| SEQ ID NO: 104 | P. aeruginosa | GTP-binding | yhbZ | General | COG0536 |
| SEQ ID NO: 106 | | protein | (obg) | function | |
| | | | | prediction | |
| SEQ ID NO: 113 | E. faecalis | acyl carrier | acpP | Not available | N/A |
| SEQ ID NO: 115 | | protein | | | |
| SEQ ID NO: 122 | E. faecalis | primosomal | dnaC | Not available | N/A |
| SEQ ID NO: 124 | | protein DnaI | | | |
| SEQ ID NO: 131 | E. faecalis | mannitol-1- | mtlD | Not available | N/A |
| SEQ ID NO: 133 | | phosphate 5- | | | |
| | | dehydrogenase | | | |
| SEQ ID NO: 140 | E. faecalis | DNA polymerase I | polA | Not available | N/A |
| SEQ ID NO: 142 | | | | | |
| SEQ ID NO: 149 | E. faecalis | adenylosuccinate | purB | Not available | N/A |
| SEQ ID NO: 151 | | lyase | | | |
| SEQ ID NO: 158 | S. aureus | adenylosuccinate | purB | Nucleotide | COG0015 |
| SEQ ID NO: 160 | | lyase | | transport and | |
| | | | | metabolism | |
| SEQ ID NO: 167 | S. pneumoniae | asparaginyl-tRNA | asnS | Translation, | COG0017 |
| SEQ ID NO: 169 | | synthetase | | ribosomal | |
| | | | | structure, and | |
| | | | | biogenesis | |
| SEQ ID NO: 176 | S. pneumoniae | dihydrofolate | dfrA | Coenzyme | COG0262 |
| SEQ ID NO: 178 | | reductase | | metabolism | |
| SEQ ID NO: 185 | E. faecalis | dnaJ protein | dnaJ | Not available | N/A |
| SEQ ID NO: 187 | | | | | |
| SEQ ID NO: 194 | P. aeruginosa | beta- | fabA | Lipid | COG0764 |
| SEQ ID NO: 196 | | hydroxydecanoyl- | | metabolism | |
| | | ACP dehydrase | | | |
| SEQ ID NO: 203 | S. aureus | mannitol-1- | mtlD | Carbohydrate | COG0246 |
| SEQ ID NO: 205 | | phosphate 5- | | transport and | |
| | | dehydrogenase | | metabolism | |
| SEQ ID NO: 212 | P. aeruginosa | dihydrofolate | dfrA | Coenzyme | COG0262 |
| SEQ ID NO: 214 | | reductase | | metabolism | |
| SEQ ID NO: 221 | S. aureus | dihydrofolate | dfrA | Coenzyme | COG0262 |
| SEQ ID NO: 223 | | reductase | | metabolism | |
| SEQ ID NO: 230 | S. aureus | replicative DNA | dnaB | DNA | COG0305 |
| SEQ ID NO: 232 | | helicase | | replication, | |
| | | | | recombination, | |
| | | | | and repair | |
| SEQ ID NO: 239 | P. aeruginosa | replicative DNA | dnaB | DNA | COG0305 |
| SEQ ID NO: 241 | | helicase | | replication, | |
| | | | | recombination, | |
| | | | | and repair | |
| SEQ ID NO: 248 | E. faecalis | cysteinyl-tRNA | cysS | Not available | N/A |
| SEQ ID NO: 250 | | synthetase | | | |
| SEQ ID NO: 257 | E. faecalis | oxidoreductase | fabG | Not available | N/A |
| SEQ ID NO: 259 | | | | | |
| SEQ ID NO: 266 | E. faecalis | cell division | ftsA | Not available | N/A |
| SEQ ID NO: 268 | | protein FtsA | | | |
| SEQ ID NO: 275 | H. pylori | UDP-3-0-acyl N- | lpxC | Cell | COG0774 |
| SEQ ID NO: 277 | | acetylglucosamine | | membrane | |
| | | deacetylase | | biogenesis | |
| SEQ ID NO: 284 | E. coli | UDP-3-O-(3- | lpxD | Cell | COG1044 |
| SEQ ID NO: 286 | | hydroxymyristoyl)- | | membrane | |
| | | glucosamine N- | | biogenesis | |
| | | acyltransferase | | | |
| SEQ ID NO: 293 | E. coli | glutamate | murI | Cell | COG0796 |
| SEQ ID NO: 295 | | racemase | | membrane | |
| | | | | biogenesis | |
| SEQ ID NO: 302 | E. faecalis | N utilization | nusA | Unknown | N/A |
| SEQ ID NO: 304 | | substance protein A | | function | |
| SEQ ID NO: 311 | H. pylori | transcription | nusA | Transcription | COG0195 |
| SEQ ID NO: 313 | | termination factor | | | |
| | | NusA | | | |
| SEQ ID NO: 320 | H. pylori | transcription | nusB | Transcription | COG0781 |
| SEQ ID NO: 322 | | termination factor | | | |
| SEQ ID NO: 329 | S. aureus | transcription | nusB | Transcription | COG0781 |
| SEQ ID NO: 331 | | termination factor | | | |
| SEQ ID NO: 338 | S. pneumoniae | ATP-dependent | pcrA | DNA | COG0210 |
| SEQ ID NO: 340 | | DNA helicase | | replication, | |
| | | | | recombination, | |
| | | | | and repair | |
| SEQ ID NO: 347 | E. faecalis | DNA-directed | rpoA | Not available | N/A |
| SEQ ID NO: 349 | | RNA polymerase, | | | |
| | | alpha subunit | | | |
| SEQ ID NO: 356 | E. faecalis | RNA polymerase | rpoD | Not available | N/A |
| SEQ ID NO: 358 | | sigma-70 factor | | | |
| | | family protein | | | |

The foregoing annotations were determined in accordance with the procedure described in EXAMPLE 17. Other biological activities of polypeptides of the invention are described herein, or will be reasonably apparent to those skilled in the art in light of the present disclosure.

A more detailed description of the biological activity for each of the polypeptides specified in the table above is set forth immediately below:

With respect to SEQ ID NO: 5 and SEQ ID NO: 7 from P. aeruginosa, the protein annotation is GTP-binding protein Era, with gene designation of era. Era is an essential GTPase that binds GTP and GDP and hydrolyses GTP to GDP. It appears to play important roles in the regulation of the cell cycle and in protein synthesis and even energy metabolism. Furthermore, the GTP-binding and hydrolysis activities of Era are essential for its biological function. Era has been observed to be very well-conserved in both Gram positive and negative bacteria. Era comprises of two distinct domains: a N-terminal domain which contains the GTP/GDP binding motifs, and a C-terminus domain that is highly conserved only among Era proteins. More recently it has been postulated that the C-terminal region of the protein is where 16S rRNA binds to this protein, and furthermore, that the binding of RNA to Era modulates the protein's GTPase activity.

With respect to SEQ ID NO: 14 and SEQ ID NO: 16 from E. faecalis, the protein annotation is short chain dehydrogenase family protein, with gene designation of scd.

With respect to SEQ ID NO: 23 and SEQ ID NO: 25 from E. faecalis, the protein annotation is glucose-inhibited division protein B, with gene designation of gidB. The initiation of chromosome replication is believed to involve two principal components, the replication origin oriC and the initiator protein DnaA. The interaction of DnaA protein and oriC results in the local unwinding of the DNA in an AT-rich region of oriC, which is the basic biochemical reaction in the initiation of replication. However, other proteins assist in this interaction, possibly two gid genes upstream of oriC. Mutations of these two genes result in glucose inhibition of division.

With respect to SEQ ID NO: 32 and SEQ ID NO: 34 from E. faecalis, the protein annotation is N utilization substance protein B, with gene designation of nusB. Further, with respect to SEQ ID NO: 320 and SEQ ID NO: 322 from H. pylori, the protein annotation is transcription termination factor, with gene designation of nusB. Still further, with respect to SEQ ID NO: 329 and SEQ ID NO: 331 from S. aureus, the protein annotation is transcription termination factor, with gene designation of nusB. This polypeptide may be abbreviated as NusB. Intrinsic transcription termination is known to play a crucial role in regulating gene expression in prokaryotes. Bacteria have several proteins that can modulate the rate of RNA chain synthesis either generally or with very specific transcription units. Bacteria have machinery that regulates ribosomal RNA transcription and employs host factors, NusA, NusB, NusE (ribosomal protein S10), and NusG. Study of these factors is important since similar control mechanisms operate in eukaryotic viruses (e.g., human immunodeficiency virus). Such factors are potentially promising drug targets.

Termination of transcription requires special terminator sequences. Termination is a distinct event in transcription because, just as the transcription complex is actively assembled, it must be actively dissembled after elongation is complete. Termination is signaled by information contained at sites in the DNA sequence being transcribed. Termination often occurs because the elongation complex is less stable when transcribing certain DNA sequences.

In those cases when termination requires a specific factor, the factor often interacts with the newly synthesized RNA, not with the DNA. For example, pause sites in the genes for rRNA should lead to transcription termination by rho, a specific regulatory protein with ATPase and helicase activity, because the nascent transcripts are not protected by ribosomes. In order to prevent such termination, the transcription complexes on rRNA genes are modified so that they do not respond to rho. This mechanism is referred to as antitermination.

One example of such an antitermination mechanism is believed to function as follows: near the promoters of rRNA genes, there is a DNA sequence known as boxA. It has been observed that, following transcription of this region, the boxA sequence of the rRNA transcript is bound by the antiterminators NusB and NusE. There is a direct and highly specific interaction between NusB and NusE, leading to the formation of a heterodimer. Neither NusB nor NusE binds boxA RNA on its own, and neither NusA nor NusG affects the interaction of the NusB-NusE complex with boxA RNA. The modified transcription complex is resistant to rho-dependent termination because interaction between rho and RNA polymerase is prevented. The presence of a binding site at the beginning of the RNA transcript serves as a tether, which restricts these antiterminator factors to a particular transcription complex. NusB, NusE, and NusG only act as antiterminators of rRNA genes, and are only effective against rho-dependent termination. The normal terminators at the ends of these genes are strong, rho-independent terminators.

The boxA sequences of the E. coli ribosomal RNA (rrn) operons are sufficient to cause RNA polymerase to read through rho-dependent transcriptional terminators. Mutations in boxA that impair its antitermination activity compromise its interaction with NusB and NusE, suggesting that NusE regulates the synthesis of ribosomal RNA in bacteria RNA containing the closely related boxA sequence from the bacteriophage lambda nutR site is not stably bound by NusB and NusE. This may explain why antitermination in phage lambda depends on the phage lambda N protein and the boxB component of the nut site, in addition to boxy. It has been shown that ribosomal protein S1 specifically binds the boxA transcriptional antiterminator RNAs of bacteriophage lambda and the E. coli rrn operons. Although S1 competes with the NusB-NusE antitermination complex for binding to boxA, it does not affect antitermination by the lambda N protein in vitro, and its role, if any, in rRNA synthesis is still unknown.

The assembly of NusB, NusG, and NusE onto the core complex involves nucleotides 2-7 of lambda boxy (CGCUCUUACACA) and is a fully cooperative process that depends on the presence of all three proteins. NusB and NusE assemble in the absence of NusG when lambda boa is altered at nucleotides 8 and 9 to create a consensus version of boxA (CGCUCUUUAACA). These experiments suggest that multiple protein-protein and protein-RNA interactions are required to convert a core antitermination complex into a complete complex.

NusB mutations that cause a loss of function or alter specificity for RNA targets are localized to surface residues and likely affect RNA-protein or protein-protein interactions. Residues that are highly conserved among homologs stabilize the protein core. In addition, NusB contains a 10 residue Arg-rich RNA-binding motif (ARM) at the N-terminus but is not sequentially homologous to any other proteins. In contrast to other known ARM-containing proteins, NusB forms a stable structure in solution in the absence of RNA.

With respect to SEQ ID NO: 41 and SEQ ID NO: 43 from E. faecalis, the protein annotation is translation elongation factor Tu, with gene designation of tufA. The tufA homologue of Enterococcus faecalis encodes translation elongation factor Tu (EF-Tu). EF-Tu is typically a guanine nucleotide-binding protein that in the GTP-bound state complexes aminoacyl-tRNA (aa-tRNA) and binds to the ribosome. Upon binding, GTP may be hydrolysed, and the GDP-bound state of EF-Tu is generally released. The structure of the E. coli EF-Tu.EF-Ts has been solved at 2.5 A resolution1. tufB orthologues have recently been identified in Vibrio cholera, Thiobacillus ferrooxidans, Yersinia pestis, Salmonella typhi Shewanella putrefaciens, Haemophilus influenzae and Pseudomonas aeruginosa in addition to Thermos thermophilus and Salmonella typhimurium. tufA and tufB homologues are likely present in all organisms. Recently, the tufA and tufB genes of E. coli have been deleted without affecting cell viability, while similar results were previously reported for S. typhimurium. A highly conserved prokaryotic protein such as EF-Tu would make an ideal target for novel antibacterials.

With respect to SEQ ID NO: 50 and SEQ ID NO: 52 from E. faecalis, the protein annotation is GTP-binding protein, with gene designation of yhbZ (obg). Further, with respect to SEQ ID NO: 104 and SEQ ID NO: 106 from P. aeruginosa, the protein annotation is GTP-binding protein, with gene designation of yhbZ (obg). In bacteria, the Obg subfamily is speculated to monitor the state of intracellular GTP levels and to serve as a switch to promote growth when bound to GTP, but not when associated with GDP. The actual targets for this switch protein are unknown at this time. However, it has been known that GTP-binding proteins are involved in cell proliferation, development, signal transduction, protein elongation, etc. and construct the GTPase superfamily, whose structures and sequence motifs (G-1 to G-5) appear to be highly conserved.

It appears that an Obg homologue have been identified in Escherichia coli, a protein known as YhbZ or alternatively ObgE. Double cross-over experiments showed that the obgE gene is essential for growth in E. coli. Further, obgE protein from E. coli had GTPase activity and DNA-binding ability. Other data suggested that ObgE is involved directly or indirectly in E. coli chromosome partitioning.

Obg of Bacillus subtilis and Obg homologues of other bacteria are believed to belong to the GTPase superfamily and have been suggested as being essential for cell growth, development and monitoring of intracellular levels of GTP. The spo0B gene of Bacillus subtilis was transcribed from a single promoter, and this transcript extended through a gene, obg, coding for a 47,668 Mr protein. The sequence of the deduced obg protein contained a region with homology to known GTP-binding proteins in the nucleotide-binding regions. The purified obg protein was shown to bind [alpha-32P]GTP in vitro and to have GTPase activity. Several attempts to inactivate the obg gene were unsuccessful, indicating that the obg gene product was essential for growth of Bacillus subtilis. Obg is immediately downstream from spo0B and is required for efficient sporulation. Because of its association with spo0B, it has been suggested that Obg has a RAS-like role in regulating the activity of the phosphorelation in the sporulation.

There is some evidence that Obg in Enterococcus faecalis is involved in essential functions related to cell growth, including influencing (i) initiation of chromosome replication, (ii) the phosphorylation state of the postexponential-phase gene regulator, Spo0A, and/or (iii) the stress activation of SigmaB in many other bacteria.

With respect to SEQ ID NO: 59 and SEQ ID NO: 61 from S. pneumoniae, the protein annotation is shikimate 5-dehydrogenase, with gene designation of aroE. Shikimate dehydrogenease, encoded by aroE, is believed to convert 3-dehydroshikimate to shikimate. This conversion is dependent on NADP and specific for shilidmate. The reaction is stereospecific, involving transfer of hydrogen from the A side of NADH. In E. coli, the functional form of the enzyme is believed to be a monomer, with a calculated Mr of 29,380.

With respect to SEQ ID NO: 68 and SEQ ID NO: 70 from P. aeruginosa, the protein annotation is conserved hypothetical protein, with gene designation of b1983. The protein encoded by the gene b1983 is extremely well conserved in a wide spectrum of bacteria. In E. coli this gene has been shown to be essential for cell viability (WO 01/48209). From genome sequence analysis of E. coli, b1983 has been localized to a region in the genome with a high number of uncharacterized genes. However, given the essentiality of b1983 and the fact that it is highly conserved, the protein product of b1983 may be a potentially good target for novel anti-microbial therapies.

With respect to SEQ ID NO: 86 and SEQ ID NO: 88 from P. aeruginosa, the protein annotation is ribosome recycling factor, with gene designation of ft. Ribosome recycling factor (RRF) is thought to be required for release of 70S ribosomes from mRNA on reaching the termination codon for the next cycle of protein synthesis. A variety of information is known on this protein, which suggest that it is a promising anti-infective target.

The RRF-encoding gene (frr) of Pseudomonas aeruginosa PAO1 has been functionally cloned by using a temperature-sensitive frr mutant of Escherichia coli and sequenced. The P. aeruginosa frr was mapped at 30 to 32 min of the P. aeruginosa chromosome. The deduced amino acid sequence of RRF showed a 64% identity to that of E. coli RRF. In an assay including E. coli polysome and elongation factor G, purified recombinant RRF of P. aeruginosa released monosomes from polysomes. This is the first case in which an RRF homologue was found to be active in heterogeneous ribosome recycling machinery.

The genes for ribosomal protein S2 (rpsB), elongation factor Ts (tsf), and UMP kinase (pyrH) are located upstream of frr in P. aeruginosa. The arrangement of the genes, rpsB-tsf-pyrH-frr, resembles those reported for E. coli and Bacillus subtilis. Even in the cyanobacterium genome, the arrangement pyrH-frr is conserved. Although RRF homologues are found in eukaryotic cells, phylogenetic analysis suggests that they were originally present within the members of the phylogenetic tree of prokaryotic RRF. This finding suggests that the ribosome recycling step catalyzed by RRF is specific for prokaryotic cells and that eukaryotic RRF is required for protein synthesis in organelles, which are believed to be phylogenetically originated from prokaryotes.

Using the ts mutant LJ14, bactericidal and bacteriostatic effect of reversible RRF inactivation of ts RRF in the lag phase and in the logarithmic phase, respectively, have been observed. In contrast, irreversible inhibition of the RRF activity by expressing plant RRF homologue in LJ14 at the permissive temperature caused bacterial death regardless of the physiological phase of bacteria. Similar effects were observed when remotely related prokaryotic RRF such as T. maritina RRF was expressed in E. coli. Bactericidal and bacteriostatic effect of removal of RRF in vivo suggests that RRF could be a target of antibacterial agents if the inhibitor of bacterial RRF does not influence eukaryotes. The frr homologues are found in eukaryotes such as yeast, human (GenBank accession #T19688, #AA004407), carrot (GenBank accession #585565), and spinach. The RRF homologue in spinach is found in organelles such as chloroplast and presumably mitochondria. In yeast, the RRF homologue is not essential because the strain without the frr homologue has been found to grow well in glucose. Hence, inhibition of eukaryotic RRF may not influence cytoplasmic protein synthesis. It is possible that a RRF inhibitor could inhibit mitochondrial protein synthesis. Indeed, it is known that antibiotics such as erythromycin, tetracycline, and chloramphenicol inhibit mitochondrial protein synthesis. Yet, such side effects do not precluse using these antibiotics for treatment.

With respect to SEQ ID NO: 95 and SEQ ID NO: 97 from S. pneumoniae, the protein annotation is N utilization substance protein A, with gene designation of nusA. Further, with respect to SEQ ID NO: 302 and SEQ ID NO: 304 from E. faecalis, the protein annotation is N utilization substance protein A, with gene designation of nusA. Still further, with respect to SEQ ID NO: 311 and SEQ ID NO: 313 from H. pylori, the protein annotation is transcription termination factor NusA, with gene designation of nusA. N utilization substance protein A (NusA) is one of several proteins that can modulate the rate of RNA chain synthesis in bacteria, either generally or with very specific transcription units. Factors of this type were first discovered as proteins involved in antitermination by N protein during bacteriophage lambda infection. Intrinsic transcription termination plays a substantial role in regulating gene expression in prokaryotes. Bacteria have analogous machinery that regulates ribosomal RNA transcription and employs host factors such as NusA, NusB, NusE (ribosomal protein S10), and NusG.

Termination is a distinct event in transcription because, just as the transcription complex is actively assembled, it is actively disassembled after elongation is complete. Termination is signaled by information contained at special terminator sequences in the DNA sequence being transcribed. Termination often occurs because the elongation complex is less stable when transcribing certain DNA sequences. Where termination requires a specific factor, the factor tyipically interacts with the newly synthesized RNA, and not usually with the DNA. Termination is often signaled by information contained at sites in the DNA sequence being transcribed. Pause sites in the genes for rRNA often lead to transcription termination by rho (a regulatory protein with ATPase and helicase activity), since the nascent transcripts are not protected by ribosomes. In order to prevent this, the transcription complexes on rRNA genes are modified so that they do not, respond to rho. In the absence of rho, transcription often does not stop, and RNA polymerase continues to copy the template strand of DNA. This event is referred to as antitermination. NusA often acts to prolong pausing at certain natural pause sites and may also have a general inhibitory effect on chain elongation that is sometimes overcome by high levels of NTPs.

Pure NusA protein typically binds reversibly to core RNA polymerase, either as a free enzyme or as part of a ternary transcription complex, but it does not usually bind to the holoenzyme. Thus, NusA will usually replace RNA polymerase sigma factor during elongation and could occupy the same binding site as sigma on the core enzyme. NusA generally helps convert RNA polymerase to the elongation form and slows down the elongation reaction, especially when the concentration of nucleoside triphosphates is low.

Negative and positive termination factors often control the efficiency of termination primarily through a direct modulation of hairpin folding and, occasionally, by changing pausing at the point of termination. Hairpin formation at the termination point relies on weak protein interactions with single-stranded RNA, which corresponds to the upstream portion of the hairpin. E. coli NusA protein destabilizes these interactions and thus promotes hairpin folding and termination. Stabilization of these contacts by phage lambda N protein leads to antitermination. By causing RNA polymerase to pause at a certain point between the start point of translation and the first intragenic, Rho-dependent terminator, NusA would allow for the ribosome to bind and initiate translation before the RNA polymerase passes into the termination region with an unprotected nascent RNA. NusA is no longer essential in strains of E. coli with mutations in the rho gene that greatly reduce the termination activity of Rho factor, and this result is consistent with the notion that a major function of NusA is to ensure efficient coupling of transcription with translation.

The association of the transcriptional antitermination protein N of bacteriophage lambda with E. coli RNA polymerase depends on nut site RNA (boxA+boxB) in the nascent transcript and the host protein, NusA. This ribonucleoprotein complex can transcribe through Rho-dependent and intrinsic termination sites located up to several hundred base pairs downstream of nut. For antitermination to occur farther downstream, this core antitermination complex must be stabilized by the host proteins NusB, NusG, and NusE. NusA protein is involved in transcription termination at the NusE attenuator in vitro. NusA is believed to act by increasing the stability of a paused transcription complex that forms at the NusE leader termination site.

The use of NusA to couple transcription with translation also occurs in the functioning of attenuators for amino acid biosynthesis operons. NusA enhances pausing at a site between the translation inititation codon and the transcriptional terminator in the trp and his operon leaders. NusA may act as a coupling factor. The structure of Mycobacterium tuberculosis NusA has been resolved to 1.7 A. Sequence and structural alignments have suggested that NusA has both S1 and KH homology regions that are thought to bind RNA. These regions are typically involved in the NusA enhancement of both termination and antitermination. There are two RNA polymerase-binding regions in NusA, one in the amino-terminal 137 amino acids and the other in the carboxy-terminal 264 amino acids; and generally the amino-terminal RNA polymerase-binding region provides a functional contact that enhances termination at an intrinsic terminator or antitermination by N. The carboxy-terminal region of NusA is also involved in the interaction with N and is important for the formation of an N-NusA-nut site or N-NusA-RNA polymerase-nut site complex; the instability of complexes lacking this carboxy-terminal region of NusA that binds N and RNA polymerase can be compensated for by the presence of the additional E. coli elongation factors, NusB, NusG, and NusE. NusA may also interfere with interactions between the nascent RNA and the C-terminal domain of the alpha subunit of RNA polymerase in E. coli transcription complexes. This effect was analyzed using the photocrosslinking nucleotide analog 5-[(4-azidophenacyl) thio]-UMP. RNA crosslinking to alpha, and loss of this crosslink when NusA was added, was observed in the presence of NusB, NusE, and NusG. Peptide mapping localized the RNA interactions to the C-terminal domain of alpha. Study of these factors is important since similar control mechanisms operate in eukaryotic viruses (e.g. human immunodeficiency virus), and NusA agonists or antagonists are likely to perturb the function of bacteria as well as viruses.

With respect to SEQ ID NO: 113 and SEQ ID NO: 115 from E. faecalis, the protein annotation is acyl carrier protein, with gene designation of acpP.

With respect to SEQ ID NO: 122 and SEQ ID NO: 124 from E. faecalis, the protein annotation is primosomal protein DnaI, with gene designation of dnaC.

With respect to SEQ ID NO: 131 and SEQ ID NO: 133 from E. faecalis, the protein annotation is mannitol-1-phosphate 5-dehydrogenase, with gene designation of mtlD. Further, with respect to SEQ ID NO: 203 and SEQ ID NO: 205 from S. aureus, the protein annotation is mannitol-1-phosphate 5-dehydrogenase, with gene designation of mtlD.

With respect to SEQ ID NO: 140 and SEQ ID NO: 142 from E. faecalis, the protein annotation is DNA polymerase I, with gene designation of polA.

With respect to SEQ ID NO: 149 and SEQ ID NO: 151 from E. faecalis, the protein annotation is adenylosuccinate lyase, with gene designation of purB. Further, with respect to SEQ ID NO: 158 and SEQ ID NO: 160 from S. aureus, the protein annotation is adenylosuccinate lyase, with gene designation of purB.

With respect to SEQ ID NO: 176 and SEQ ID NO: 178 from S. pneumoniae, the protein annotation is dihydrofolate reductase, with gene designation of dfrA. Further, with respect to SEQ ID NO: 212 and SEQ ID NO: 214 from P. aeruginosa, the protein annotation is dihydrofolate reductase, with gene designation of dfrA. Still further, with respect to SEQ ID NO: 221 and SEQ ID NO: 223 from S. aureus, the protein annotation is dihydrofolate reductase, with gene designation of dfrA.

With respect to SEQ ID NO: 185 and SEQ ID NO: 187 from E. faecalis, the protein annotation is dnaJ protein, with gene designation of dnaJ.

With respect to SEQ ID NO: 77 and SEQ ID NO: 79 from P. aeruginosa, the protein annotation is beta-ketoacyl-ACP synthase I, with gene designation of fabB. Further, with respect to SEQ ID NO: 194 and SEQ ID NO: 196 from P. aeruginosa, the protein annotation is beta-hydroxydecanoyl-ACP dehydrase, with gene designation of fabA. Still further, with respect to SEQ ID NO: 257 and SEQ ID NO: 259 from E. faecalis, the protein annotation is oxidoreductase, with gene designation of fabG. Fatty acid biosynthesis is believed to be carried out by the ubiquitous fatty-acid synthase (FAS) system. The first step in the fatty acid biosynthetic cycle appears to be the condensation of malonyl-acyl carrier protein (or ““malonyl-ACP”) with acetyl-CoA by FabH. Prior to this, malonyl-ACP appears to be synthesized from ACP and malonyl-CoA by FabD, malonyl CoA:ACP transacylase. In subsequent rounds, malonyl-ACP may be condensed with the growing-chain acyl-ACP. These condensation reactions may be carried out by beta-ketoacyl-ACP-synthase I, II, or III, encoded by fabB, fabF and fabH. The second step in the elongation cycle appears to be ketoester reduction by NADPH-dependent beta.-ketoacyl-ACP reductase (FabG). Subsequent dehydration by beta.-hydroxyacyl-ACP dehydrase (either FabA or FabZ) may leads to trans-2-enoyl-ACP, which in turn may be converted to acyl-ACP by enoyl-ACP reductase (FabI). Further rounds of this cycle, adding two carbon atoms per cycle, eventually lead to palmitoyl-ACP, whereupon the cycle may be inhibited by feedback inhibition of FabH and FabI by palmitoyl-ACP.

Yeast and vertebrates generally employ the type I FAS system, whereby fatty acid biosynthesis has been observed to proceed via a single multifunctional polypeptide complex. In contrast, in most bacteria and plants a type II FAS system is employed, wherein each of the reactions may be catalyzed by distinct monofunctional enzymes and the ACP is a discrete protein. Thus, there appears to be considerable potential for selective inhibition of the bacterial FAS systems. The absolute requirement of type II FAS for bacterial viability, together with its major differences from the mammalian system, suggests that enzymes in this pathway may be good targets for broad spectrum antibacterial compounds.

With respect to SEQ ID NO: 230 and SEQ ID NO: 232 from S. aureus, the protein annotation is replicative DNA helicase, with gene designation of dnaB. Further, with respect to SEQ ID NO: 239 and SEQ ID) NO: 241 from P. aeruginosa, the protein annotation is replicative DNA helicase, with gene designation of dnaB.

With respect to SEQ ID NO: 167 and SEQ ID NO: 169 from S. pneumoniae, the protein annotation is asparaginyl-tRNA-synthetase, with gene designation of asnS. Further, with respect to SEQ ID NO: 248 and SEQ ID NO: 250 from E. faecalis, the protein annotation is cysteinyl-tRNA synthetase, with gene designation of cysS. Proteins may be encoded by a DNA or RNA template. Amino acids have been observed to be activated and transported to the ribosome via attachment to tRNA, an adaptor molecule. Amino acid activation and subsequent linkage to tRNA appear to be catalyzed by aminoacyl-tRNA synthetases. When a tRNA molecule recognizes its correct codon on the ribosome bound mRNA, the attached amino acid is released and added onto the growing polypeptide chain, apparently regardless of the amino acid identity. Thus, while tRNA may recognize the correct codon on the mRNA, tRNA itself does not appear to be responsible for ensuring that the correct amino acid is attached to it, but rather the aminoacyl-tRNA synthetases.

In aminoacyl-tRNA synthetases, the acylation site has been observed as the site where amino acid substrates are bound, activated, and attached to tRNA. The aminoacyl-tRNA synthetase catalyzed aminoacylation of tRNA has been observed to proceed through two steps. First, ATP appears to activate the amino acid, forming an enzyme-bound aminoacyl-adenylate intermediate and inorganic pyrophosphate. Secondly, the amino acid moiety may be transferred to either the 2′OH or 3′OH of the terminal adenosine of the tRNA molecule to generate aminoacyl-tRNA and AMP.

In addition to the acylation site, most aminoacyl-tRNA synthetases appear to contain a hydrolytic site. Acylation sites apparently reject amino acid substrates that are larger than the correct amino acid substrate because there is insufficient room in the acylation site for the amino acids to bind, be activated, and become attached to tRNA. Hydrolytic sites appear to destroy activated intermediates that are smaller than the correct activated intermediate. However, some aminoacyl-tRNA synthetases do not have a hydrolytic site, and instead appear to discriminate between correct and incorrect amino acids via another mechanism. The appropriate tRNA substrate may be recognized by the aminoacyl-tRNA synthetases in several ways, such as via the anticodon loop, acceptor stem, or another unique identifying characteristic. By their apparent selectivity in recognition of both the amino acid substrates and the prospective tRNA acceptors, aminoacyl-tRNA synthetases are throught to establish the basis for the fidelity of protein synthesis from a nucleic acid template.

Because aminoacyl-tRNA synthetases appear to be universal and essential for cell viability, potent aminoacyl-tRNA synthetase inhibitors that are also selective for pathogens may be very attractive drug targets. The world's most widely used topical antibiotic, mupirocin, is an aminoacyl-tRNA synthetase inhibitor. Mupirocin inhibits eubacterial and archaeal isoleucyl-tRNA synthetases, but is 1000-fold less potent against eukaryotic isoleucyl-tRNA synthetase. Mupirocin illustrates the clinical application of a potent, highly selective bacterial aminoacyl-tRNA synthetase inhibitor.

With respect to SEQ ID NO: 266 and SEQ ID NO: 268 from E. faecalis, the protein annotation is cell division protein FtsA, with gene designation of ftsA. Cellular constituents are divided into two separate daughter cells following or during mitosis during the process of cytokinesis. The cell envelope has been observed to invaginate circumferentially at the cell division site during E. coli cytokinesis. The “Z ring” is thought to be a cytoskeletal scaffold needed for the assembly of all the other known division proteins at the division site in order for cytokinesis to proceed. The coordinated invagination is believed to require at least nine proteins to localize to the division site before cell division can occur. Firstly, the bacterial tubulin homologue FtsZ has been observed to polymerize into the Z ring at the division site. ZipA and FtsA have been observed to subsequently localize independently to the division site by binding to the FtsZ C-terminus. Following ZipA and FtsA localization, the remaining division proteins are thought to bind in the following order, FtsK, FtsQ, FtsL, FtsW, FtsI and FtsN. FtsA plays a role in the recruitment of FtsK and therefore also in the recruitment of all downstream division proteins.

With respect to SEQ ID NO: 275 and SEQ ID NO: 277 from H. pylori, the protein annotation is UDP-3-O-acyl N-acetylglucosamine deacetylase, with gene designation of lpxC.

With respect to SEQ ID NO: 284 and SEQ ID NO: 286 from E. coli, the protein annotation is UDP-3-O-(3-hydroxymyristoyl)-glucosamine N-acyltransferase, with gene designation of lpxD.

With respect to SEQ ID NO: 293 and SEQ ID NO: 295 from E. coli, the protein annotation is glutamate racemase, with gene designation of murI. Peptidoglycan, a component of the bacterial cell wall, is thought to play a critical role in protecting bacteria against osmotic lysis. It is comprised of linearly repeating disaccharide chains cross-linked by short peptide bridges. Glutamate racemase, a product of the murI gene, has been observed to catalyze the interconversion of glutamate enantiomers in a cofactor-independent manner to provide D-glutamate required for peptidoglycan synthesis. Structural studies on glutamate racemase have indicated that two cysteines in the active site may be used as acid/base catalysts during the reaction. The crystal structure of murI from Aquifex pyrophilus has been determined to 2.3 Å, and reveals that the enzyme may be comprised of a dimer. Each monomer comprises two alpha/beta fold domains, which appear to have a unique fold as compared to other racemases or enolases. Because glutamate racemase is essential for cell viability and is highly conserved in bacteria, it may be a potential target for the development of novel anti-bacterial therapeutic molecules.

With respect to SEQ ID NO: 338 and SEQ ID NO: 340 from S. pneumoniae, the protein annotation is ATP-dependent DNA helicase, with gene designation of pcrA.

With respect to SEQ ID NO: 347 and SEQ ID NO: 349 from E. faecalis, the protein annotation is DNA-directed RNA polymerase, alpha subunit, with gene designation of rpoA. DNA-dependent RNA polymerase (RNAP) is thought to be an essential and universally conserved protein in bacterial synthesis of RNA during transcription. The enzyme comprises four subunits, with a molecular mass of around 400 kDa. One of the four subunits of this enzyme is encoded by the gene rpoA. Another subunit, namely, beta-prime, is the target of Rifampicin, a broad spectrum antibiotic.

With respect to SEQ ID NO: 356 and SEQ ID NO: 358 from E. faecalis, the protein annotation is RNA polymerase sigma-70 factor family protein, with gene designation of rpoD. DNA-dependent RNA polymerase (RNAP) is an essential and universally conserved protein in bacteria, and appears to be involved in the synthesis of RNA during transcription. The enzyme has been observed to catalyze phosphodiester bond formation during RNA synthesis. The enzyme comprises four subunits, with a molecular mass of around 400 kDa. Taken together, the β and the β′ subunits appear to constitute 70% of the enzyme mass and carry out most of the functions of the enzyme. Recent work has suggested that the β′ subunit is required for the formation of the binding pocket for the antibiotic Rifampicin to bind to the enzyme. The gene rpoC encodes the β′ subunit of the enzyme complex, while the the rpoD gene encodes the sigma-70 factor. Promoter recognition depends on the sigma-70 factor, which interacts with RNAP to form the holoenzyme. The crystal structure of the Thermus aquaticus RNA polymerase has been deciphered at 3.3 Å resolution. Determining the structures of RNAPs from other pathogenic microorganisms may aid in the design of novel therapeutic agents.

For all of the foregoing reasons, the polypeptides of the present invention are potentially valuable targets of therapeutics and diagnostics.

3. Nucleic Acids of the Invention

One aspect of the invention pertains to isolated nucleic acids of the invention. For example, the present invention contemplates an isolated nucleic acid comprising (a) a subject nucleic acid sequence, (b) a nucleotide sequence at least 80% identical to the subject nucleic acid sequence, (c) a nucleotide sequence that hybridizes under stringent conditions to the subject nucleic acid sequence, or (d) the complement of the nucleotide sequence of (a), (b) or (c). In certain embodiments, nucleic acids of the invention may be labeled, with for example, a radioactive, chemiluminescent or fluorescent label.

It may be the case that the nucleic acid sequence for a nucleic acid of the invention predicted from the publicly available genomic information differs from the nucleic acid sequence determined experimentally as described below. For example, in the case of GTP-binding protein Era (era) from P. aeruginosa, SEQ ID NO: 6 is determined experimentally, and SEQ ID NO: 4 obtained as described in EXAMPLE 1. In such a case, the present invention contemplates the specific nucleic acid sequences of SEQ ID NO: 4 and SEQ ID NO: 6, and variants thereof, as well as any differences in the applicable amino acid sequences encoded thereby.

In another aspect, the present invention contemplates an isolated nucleic acid that specifically hybridizes under stringent conditions to at least ten nucleotides of a subject nucleic acid sequence, or the complement thereof, which nucleic acid can specifically detect or amplify the same subject nucleic acid sequence, or the complement thereof. In yet another aspect, the present invention contemplates such an isolated nucleic acid comprising a nucleotide sequence encoding a fragment of a subject amino acid sequence at least 8 residues in length. The present invention further contemplates a method of hybridizing an oligonucleotide with a nucleic acid of the invention comprising: (a) providing a single-stranded oligonucleotide at least eight nucleotides in length, the oligonucleotide being complementary to a portion of a nucleic acid of the invention; and (b) contacting the oligonucleotide with a sample comprising a nucleic acid of the acid under conditions that permit hybridization of the oligonucleotide with the nucleic acid of the invention.

Isolated nucleic acids which differ from the nucleic acids of the invention due to degeneracy in the genetic code are also within the scope of the invention. For example, a number of amino acids are designated by more than one triplet. Codons that specify the same amino acid, or synonyms (for example, CAU and CAC are synonyms for histidine) may result in “silent” mutations which do not affect the amino acid sequence of the protein. However, it is expected that DNA sequence polymorphisms that do lead to changes in the amino acid sequences of the polypeptides of the invention will exist. One skilled in the art will appreciate that these variations in one or more nucleotides (from less than 1% up to about 3 or 5% or possibly more of the nucleotides) of the nucleic acids encoding a particular protein of the invention may exist among a given species due to natural allelic variation. Any and all such nucleotide variations and resulting amino acid polymorphisms are within the scope of this invention.

Bias in codon choice within genes in a single species appears related to the level of expression of the protein encoded by that gene. Accordingly, the invention encompasses nucleic acid sequences which have been optimized for improved expression in a host cell by altering the frequency of codon usage in the nucleic acid sequence to approach the frequency of preferred codon usage of the host cell. Due to codon degeneracy, it is possible to optimize the nucleotide sequence without affecting the amino acid sequence of an encoded polypeptide. Accordingly, the instant invention relates to any nucleotide sequence that encodes all or a substantial portion of a subject amino acid sequence or other polypeptides of the invention.

The present invention pertains to nucleic acids encoding proteins derived from the same pathogenic species as a polypeptide of the invention and which have amino acid sequences evolutionarily related to such polypeptide, wherein “evolutionarily related to”, refers to proteins having different amino acid sequences which have arisen naturally (e.g. by allelic variance or by differential splicing), as well as mutational variants of the proteins of the invention which are derived, for example, by combinatorial mutagenesis.

Fragments of the polynucleotides of the invention encoding a biologically active portion of a subject amino acid sequence or other polypeptides of the invention are also within the scope of the invention. As used herein, a fragment of a nucleic acid of the invention encoding an active portion of a polypeptide of the invention refers to a nucleotide sequence having fewer nucleotides than the nucleotide sequence encoding the full length amino acid sequence of a polypeptide of the invention, and which encodes a polypeptide which retains at least a portion of a biological activity of the full-length protein as defined herein, or alternatively, which is functional as a modulator of a biological activity of the full-length protein. For example, such fragments include a polypeptide containing a domain of the full-length protein from which the polypeptide is derived that mediates the interaction of the protein with another molecule (e.g., polypeptide, DNA, RNA, etc.). In another embodiment, the present invention contemplates an isolated nucleic acid that encodes a polypeptide having a biological activity of a subject amino acid sequence.

Nucleic acids within the scope of the invention may also contain linker sequences, modified restriction endonuclease sites and other sequences useful for molecular cloning, expression or purification of such recombinant polypeptides.

A nucleic acid encoding a polypeptide of the invention may be obtained from mRNA or genomic DNA from any organism in accordance with protocols described herein, as well as those generally known to those skilled in the art. A cDNA encoding a polypeptide of the invention, for example, may be obtained by isolating total mRNA from an organism, e.g. a bacteria, virus, mammal, etc. Double stranded cDNAs may then be prepared from the total mRNA, and subsequently inserted into a suitable plasmid or bacteriophage vector using any one of a number of known techniques. A gene encoding a polypeptide of the invention may also be cloned using established polymerase chain reaction techniques in accordance with the nucleotide sequence information provided by the invention. In one aspect, the present invention contemplates a method for amplification of a nucleic acid of the invention, or a fragment thereof, comprising: (a) providing a pair of single stranded oligonucleotides, each of which is at least eight nucleotides in length, complementary to sequences of a nucleic acid of the invention, and wherein the sequences to which the oligonucleotides are complementary are at least ten nucleotides apart; and (b) contacting the oligonucleotides with a sample comprising a nucleic acid comprising the nucleic acid of the invention under conditions which permit amplification of the region located between the pair of oligonucleotides, thereby amplifying the nucleic acid.

Another aspect of the invention relates to the use of nucleic acids of the invention in “antisense therapy”. As used herein, antisense therapy refers to administration or in situ generation of oligonucleotide probes or their derivatives which specifically hybridize or otherwise bind under cellular conditions with the cellular mRNA and/or genomic DNA encoding one of the polypeptides of the invention so as to inhibit expression of that polypeptide, e.g. by inhibiting transcription and/or translation. The binding may be by conventional base pair complementarity, or, for example, in the case of binding to DNA duplexes, through specific interactions in the major groove of the double helix. In general, antisense therapy refers to the range of techniques generally employed in the art, and includes any therapy which relies on specific binding to oligonucleotide sequences.

An antisense construct of the present invention may be delivered, for example, as an expression plasmid which, when transcribed in the cell, produces RNA which is complementary to at least a unique portion of the mRNA which encodes a polypeptide of the invention. Alternatively, the antisense construct may be an oligonucleotide probe which is generated ex vivo and which, when introduced into the cell causes inhibition of expression by hybridizing with the mRNA and/or genomic sequences encoding a polypeptide of the invention. Such oligonucleotide probes may be modified oligonucleotides which are resistant to endogenous nucleases, e.g. exonucleases and/or endonucleases, and are therefore stable in vivo. Exemplary nucleic acid molecules for use as antisense oligonucleotides are phosphoramidate, phosphothioate and methylphosphonate analogs of DNA (see also U.S. Pat. Nos. 5,176,996; 5,264,564; and 5,256,775). Additionally, general approaches to constructing oligomers useful in antisense therapy have been reviewed, for example, by van der Krol et al., (1988) Biotechniques 6:958-976; and Stein et al., (1988) Cancer Res 48:2659-2668.

In a further aspect, the invention provides double stranded small interfering RNAs (siRNAs), and methods for administering the same. siRNAs decrease or block gene expression. While not wishing to be bound by theory, it is generally thought that siRNAs inhibit gene expression by mediating sequence specific mRNA degradation. RNA interference (RNAi) is the process of sequence-specific, post-transcriptional gene silencing, particularly in animals and plants, initiated by double-stranded RNA (dsRNA) that is homologous in sequence to the silenced gene (Elbashir et al. Nature 2001; 411(6836): 494-8). Accordingly, it is understood that siRNAs and long dsRNAs having substantial sequence identity to all or a portion of a subject nucleic acid sequence may be used to inhibit the expression of a nucleic acid of the invention, and particularly when the polynucleotide is expressed in a mammalian or plant cell.

The nucleic acids of the invention may be used as diagnostic reagents to detect the presence or absence of the target DNA or RNA sequences to which they specifically bind, such as for determining the level of expression of a nucleic acid of the invention. In one aspect, the present invention contemplates a method for detecting the presence of a nucleic acid of the invention or a portion thereof in a sample, the method comprising: (a) providing an oligonucleotide at least eight nucleotides in length, the oligonucleotide being complementary to a portion of a nucleic acid of the invention; (b) contacting the oligonucleotide with a sample comprising at least one nucleic acid under conditions that permit hybridization of the oligonucleotide with a nucleic acid comprising a nucleotide sequence complementary thereto; and (c) detecting hybridization of the oligonucleotide to a nucleic acid in the sample, thereby detecting the presence of a nucleic acid of the invention or a portion thereof in the sample. In another aspect, the present invention contemplates a method for detecting the presence of a nucleic acid of the invention or a portion thereof in a sample, the method comprising: (a) providing a pair of single stranded oligonucleotides, each of which is at least eight nucleotides in length, complementary to sequences of a nucleic acid of the invention, and wherein the sequences to which the oligonucleotides are complementary are at least ten nucleotides apart; and (b) contacting the oligonucleotides with a sample comprising at least one nucleic acid under hybridization conditions; (c) amplifying the nucleotide sequence between the two oligonucleotide primers; and (d) detecting the presence of the amplified sequence, thereby detecting the presence of a nucleic acid comprising the nucleic acid of the invention or a portion thereof in the sample.

In another aspect of the invention, the subject nucleic acid is provided in an expression vector comprising a nucleotide sequence encoding a polypeptide of the invention and operably linked to at least one regulatory sequence. It should be understood that the design of the expression vector may depend on such factors as the choice of the host cell to be transformed and/or the type of protein desired to be expressed. The vector's copy number, the ability to control that copy number and the expression of any other protein encoded by the vector, such as antibiotic markers, should be considered.

The subject nucleic acids may be used to cause expression and over-expression of a polypeptide of the invention in cells propagated in culture, e.g. to produce proteins or polypeptides, including fusion proteins or polypeptides.

This invention pertains to a host cell transfected with a recombinant gene in order to express a polypeptide of the invention. The host cell may be any prokaryotic or eukaryotic cell. For example, a polypeptide of the invention may be expressed in bacterial cells, such as E. coli, insect cells (baculovirus), yeast, or mammalian cells. In those instances when the host cell is human, it may or may not be in a live subject. Other suitable host cells are known to those skilled in the art. Additionally, the host cell may be supplemented with tRNA molecules not typically found in the host so as to optimize expression of the polypeptide. Other methods suitable for maximizing expression of the polypeptide will be known to those in the art.

The present invention further pertains to methods of producing the polypeptides of the invention. For example, a host cell transfected with an expression vector encoding a polypeptide of the invention may be cultured under appropriate conditions to allow expression of the polypeptide to occur. The polypeptide may be secreted and isolated from a mixture of cells and medium containing the polypeptide. Alternatively, the polypeptide may be retained cytoplasmically and the cells harvested, lysed and the protein isolated.

A cell culture includes host cells, media and other byproducts. Suitable media for cell culture are well known in the art. The polypeptide may be isolated from cell culture medium, host cells, or both using techniques known in the art for purifying proteins, including ion-exchange chromatography, gel filtration chromatography, ultrafiltration, electrophoresis, and immunoaffinity purification with antibodies specific for particular epitopes of a polypeptide of the invention.

Thus, a nucleotide sequence encoding all or a selected portion of polypeptide of the invention, may be used to produce a recombinant form of the protein via microbial or eukaryotic cellular processes. Ligating the sequence into a polynucleotide construct, such as an expression vector, and transforming or transfecting into hosts, either eukaryotic (yeast, avian, insect or mammalian) or prokaryotic (bacterial cells), are standard procedures. Similar procedures, or modifications thereof, may be employed to prepare recombinant polypeptides of the invention by microbial means or tissue-culture technology.

Expression vehicles for production of a recombinant protein include plasmids and other vectors. For instance, suitable vectors for the expression of a polypeptide of the invention include plasmids of the types: pBR322-derived plasmids, pEMBL-derived plasmids, pEX-derived plasmids, pBTac-derived plasmids and pUC-derived plasmids for expression in prokaryotic cells, such as E. coli.

A number of vectors exist for the expression of recombinant proteins in yeast. For instance, YEP24, YIP5, YEP51, YEP52, pYES2, and YRP17 are cloning and expression vehicles useful in the introduction of genetic constructs into S. cerevisiae (see, for example, Broach et al., (1983) in Experimental Manipulation of Gene Expression, ed. M. Inouye Academic Press, p. 83). These vectors may replicate in E. coli due the presence of the pBR322 ori, and in S. cerevisiae due to the replication determinant of the yeast 2 micron plasmid. In addition, drug resistance markers such as ampicillin may be used.

In certain embodiments, mammalian expression vectors contain both prokaryotic sequences to facilitate the propagation of the vector in bacteria, and one or more eukaryotic transcription units that are expressed in eukaryotic cells. The pcDNAI/amp, pcDNAI/neo, pRc/CMV, pSV2gpt, pSV2neo, pSV2-dhfr, pTk2, pRSVneo, pMSG, pSVT7, pko-neo and pHyg derived vectors are examples of mammalian expression vectors suitable for transfection of eukaryotic cells. Some of these vectors are modified with sequences from bacterial plasmids, such as pBR322, to facilitate replication and drug resistance selection in both prokaryotic and eukaryotic cells. Alternatively, derivatives of viruses such as the bovine papilloma virus (BPV-1), or Epstein-Barr virus (pHEBo, pREP-derived and p205) can be used for transient expression of proteins in eukaryotic cells. The various methods employed in the preparation of the plasmids and transformation of host organisms are well known in the art. For other suitable expression systems for both prokaryotic and eukaryotic cells, as well as general recombinant procedures, see Molecular Cloning A Laboratory Manual, 2nd Ed., ed. by Sambrook, Fritsch and Maniatis (Cold Spring Harbor Laboratory Press, 1989) Chapters 16 and 17. In some instances, it may be desirable to express the recombinant protein by the use of a baculovirus expression system. Examples of such baculovirus expression systems include pVL-derived vectors (such as pVL1392, pVL1393 and pVL941), pAcUW-derived vectors (such as pAcUW1), and pBlueBac-derived vectors (such as the β-gal containing pBlueBac III).

In another variation, protein production may be achieved using in vitro translation systems. In vitro translation systems are, generally, a translation system which is a cell-free extract containing at least the minimum elements necessary for translation of an RNA molecule into a protein. An in vitro translation system typically comprises at least ribosomes, tRNAs, initiator methionyl-tRNAMet, proteins or complexes involved in translation, e.g., eIF2, eIF3, the cap-binding (CB) complex, comprising the cap-binding protein (CBP) and eukaryotic initiation factor 4F (eIF4F). A variety of in vitro translation systems are well known in the art and include commercially available kits. Examples of in vitro translation systems include eukaryotic lysates, such as rabbit reticulocyte lysates, rabbit oocyte lysates, human cell lysates, insect cell lysates and wheat germ extracts. Lysates are commercially available from manufacturers such as Promega Corp., Madison, Wis.; Stratagene, La Jolla, Calif.; Amersham, Arlington Heights, Ill.; and GIBCO/BRL, Grand Island, N.Y. In vitro translation systems typically comprise macromolecules, such as enzymes, translation, initiation and elongation factors, chemical reagents, and ribosomes. In addition, an in vitro transcription system may be used. Such systems typically comprise at least an RNA polymerase holoenzyme, ribonucleotides and any necessary transcription initiation, elongation and termination factors. In vitro transcription and translation may be coupled in a one-pot reaction to produce proteins from one or more isolated DNAs.

When expression of a carboxy terminal fragment of a polypeptide is desired, i.e. a truncation mutant, it may be necessary to add a start codon (ATG) to the oligonucleotide fragment containing the desired sequence to be expressed. It is well known in the art that a methionine at the N-terminal position may be enzymatically cleaved by the use of the enzyme methionine aminopeptidase (MAP). MAP has been cloned from E. coli (Ben-Bassat et al., (1987) J. Bacteriol. 169:751-757) and Salmonella typhimurium and its in vitro activity has been demonstrated on recombinant proteins (Miller et al., (1987) PNAS USA 84:2718-1722). Therefore, removal of an N-terminal methionine, if desired, may be achieved either in vivo by expressing such recombinant polypeptides in a host which produces MAP (e.g., E. coli or CM89 or S. cerevisiae), or in vitro by use of purified MAP (e.g., procedure of Miller et al.).

Coding sequences for a polypeptide of interest may be incorporated as a part of a fusion gene including a nucleotide sequence encoding a different polypeptide. The present invention contemplates an isolated nucleic acid comprising a nucleic acid of the invention and at least one heterologous sequence encoding a heterologous peptide linked in frame to the nucleotide sequence of the nucleic acid of the invention so as to encode a fusion protein comprising the heterologous polypeptide. The heterologous polypeptide may be fused to (a) the C-terminus of the polypeptide encoded by the nucleic acid of the invention, (b) the N-terminus of the polypeptide, or (c) the C-terminus and the N-terminus of the polypeptide. In certain instances, the heterologous sequence encodes a polypeptide permitting the detection, isolation, solubilization and/or stabilization of the polypeptide to which it is fused. In still other embodiments, the heterologous sequence encodes a polypeptide selected from the group consisting of a polyHis tag, myc, HA, GST, protein A, protein G, calmodulin-binding peptide, thioredoxin, maltose-binding protein, poly arginine, poly His-Asp, FLAG, a portion of an immunoglobulin protein, and a transcytosis peptide.

Fusion expression systems can be useful when it is desirable to produce an immunogenic fragment of a polypeptide of the invention. For example, the VP6 capsid protein of rotavirus may be used as an immunologic carrier protein for portions of polypeptide, either in the monomeric form or in the form of a viral particle. The nucleic acid sequences corresponding to the portion of a polypeptide of the invention to which antibodies are to be raised may be incorporated into a fusion gene construct which includes coding sequences for a late vaccinia virus structural protein to produce a set of recombinant viruses expressing fusion proteins comprising a portion of the protein as part of the virion. The Hepatitis B surface antigen may also be utilized in this role as well. Similarly, chimeric constructs coding for fusion proteins containing a portion of a polypeptide of the invention and the poliovirus capsid protein may be created to enhance immunogenicity (see, for example, EP Publication NO: 0259149; and Evans et al., (1989) Nature 339:385; Huang et al., (1988) J. Virol. 62:3855; and Schlienger et al., (1992) J. Virol. 66:2).

Fusion proteins may facilitate the expression and/or purification of proteins. For example, a polypeptide of the invention may be generated as a glutathione-S-transferase (GST) fusion protein. Such GST fusion proteins may be used to simplify purification of a polypeptide of the invention, such as through the use of glutathione-derivatized matrices (see, for example, Current Protocols in Molecular Biology, eds. Ausubel et al., (N.Y.: John Wiley & Sons, 1991)). In another embodiment, a fusion gene coding for a purification leader sequence, such as a poly-(His)/enterokinase cleavage site sequence at the N-terminus of the desired portion of the recombinant protein, may allow purification of the expressed fusion protein by affinity chromatography using a Ni²⁺ metal resin. The purification leader sequence may then be subsequently removed by treatment with enterokinase to provide the purified protein (e.g., see Hochuli et al., (1987) J. Chromatography 411: 177; and Janknecht et al., PNAS USA 88:8972).

Techniques for making fusion genes are well known. Essentially, the joining of various DNA fragments coding for different polypeptide sequences is performed in accordance with conventional techniques, employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene may be synthesized by conventional techniques including automated DNA synthesizers. Alternatively, PCR amplification of gene fragments may be carried out using anchor primers which give rise to complementary overhangs between two consecutive gene fragments which may subsequently be annealed to generate a chimeric gene sequence (see, for example, Current Protocols in Molecular Biology, eds. Ausubel et al., John Wiley & Sons: 1992).

The present invention further contemplates a transgenic non-human animal having cells which harbor a transgene comprising a nucleic acid of the invention.

In other embodiments, the invention provides for nucleic acids of the invention immobilized onto a solid surface, including, plates, microtiter plates, slides, beads, particles, spheres, films, strands, precipitates, gels, sheets, tubing, containers, capillaries, pads, slices, etc. The nucleic acids of the invention may be immobilized onto a chip as part of an array. The array may comprise one or more polynucleotides of the invention as described herein. In one embodiment, the chip comprises one or more polynucleotides of the invention as part of an array of polynucleotide sequences from the same pathogenic species as such polynucleotide(s).

In still other embodiments, the invention comprises the sequence of a nucleic acid of the invention in computer readable format. The invention also encompasses a database comprising the sequence of a nucleic acid of the invention.

4. Homology Searching of Nucleotide and Polypeptide Sequences

The nucleotide or amino acid sequences of the invention, including those set forth in the appended Figures, may be used as query sequences against databases such as GenBank, SwissProt, PDB, BLOCKS, and Pima II. These databases contain previously identified and annotated sequences that may be searched for regions of homology (similarity) using BLAST, which stands for Basic Local Alignment Search Tool (Altschul S F (1993) J Mol Evol 36:290-300; Altschul, S F et al (1990) J Mol Biol 215:403-10).

BLAST produces alignments of both nucleotide and amino acid sequences to determine sequence similarity. Because of the local nature of the alignments, BLAST is especially useful in determining exact matches or in identifying homologs which may be of prokaryotic (bacterial) or eukaryotic (animal, fungal or plant) origin. Other algorithms such as the one described in Smith, R. F. and T. F. Smith (1992; Protein Engineering 5:35-51) may be used when dealing with primary sequence patterns and secondary structure gap penalties. In the usual course using BLAST, sequences have lengths of at least 49 nucleotides and no more than 12% uncalled bases (where N is recorded rather than A, C, G, or T).

The BLAST approach, as detailed in Karlin and Altschul (1993; Proc Nat Acad Sci 90:5873-7) searches matches between a query sequence and a database sequence, to evaluate the statistical significance of any matches found, and to report only those matches which satisfy the user-selected threshold of significance. The threshold is typically set at about 10-25 for nucleotides and about 3-15 for peptides.

5. Analysis of Protein Properties

(a) Analysis of Proteins by Mass Spectrometry

Typically, protein characterization by mass spectroscopy first requires protein isolation followed by either chemical or enzymatic digestion of the protein into smaller peptide fragments, whereupon the peptide fragments may be analyzed by mass spectrometry to obtain a peptide map. Mass spectrometry may also be used to identify post-translational modifications (e.g., phosphorylation, etc.) of a polypeptide.

Various mass spectrometers may be used within the present invention. Representative examples include: triple quadrupole mass spectrometers, magnetic sector instruments (magnetic tandem mass spectrometer, JEOL, Peabody, Mass.), ionspray mass spectrometers (Bruins et al., Anal Chem. 59:2642-2647, 1987), electrospray mass spectrometers (including tandem, nano- and nano-electrospray tandem) (Fenn et al., Science 246:64-71, 1989), laser desorption time-of-flight mass spectrometers (Karas and Hillenkamp, Anal. Chem. 60:2299-2301, 1988), and a Fourier Transform Ion Cyclotron Resonance Mass Spectrometer (Extrel Corp., Pittsburgh, Mass.).

MALDI ionization is a technique in which samples of interest, in this case peptides and proteins, are co-crystallized with an acidified matrix. The matrix is typically a small molecule that absorbs at a specific wavelength, generally in the ultraviolet (UV) range, and dissipates the absorbed energy thermally. Typically a pulsed laser beam is used to transfer energy rapidly (i.e., a few ns) to the matrix. This transfer of energy causes the matrix to rapidly dissociate from the MALDI plate surface and results in a plume of matrix and the co-crystallized analytes being transferred into the gas phase. MALDI is considered a “soft-ionization” method that typically results in singly-charged species in the gas phase, most often resulting from a protonation reaction with the matrix. MALDI may be coupled in-line with time of flight (TOF) mass spectrometers. TOF detectors are based on the principle that an analyte moves with a velocity proportional to its mass. Analytes of higher mass move slower than analytes of lower mass and thus reach the detector later than lighter analytes. The present invention contemplates a composition comprising a polypeptide of the invention and a matrix suitable for mass spectrometry. In certain instances, the matrix is a nicotinic acid derivative or a cinnamic acid derivative.

MALDI-TOF MS is easily performed with modern mass spectrometers. Typically the samples of interest, in this case peptides or proteins, are mixed with a matrix and spotted onto a polished stainless steel plate (MALDI plate). Commercially available MALDI plates can presently hold up to 1536 samples per plate. Once spotted with sample, the MALDI sample plate is then introduced into the vacuum chamber of a MALDI mass spectrometer. The pulsed laser is then activated and the mass to charge ratios of the analytes are measured utilizing a time of flight detector. A mass spectrum representing the mass to charge ratios of the peptides/proteins is generated.

As mentioned above, MALDI can be utilized to measure the mass to charge ratios of both proteins and peptides. In the case of proteins, a mixture of intact protein and matrix are co-crystallized on a MALDI target (Karas, M. and Hillenkamp, F. Anal. Chem. 1988, 60 (20) 2299-2301). The spectrum resulting from this analysis is employed to determine the molecular weight of a whole protein. This molecular weight can then be compared to the theoretical weight of the protein and utilized in characterizing the analyte of interest, such as whether or not the protein has undergone post-translational modifications (e.g., example phosphorylation).

In certain embodiments, MALDI mass spectrometry is used for determination of peptide maps of digested proteins. The peptide masses are measured accurately using a MALDI-TOF or a MALDI-Q-Star mass spectrometer, with detection precision down to the low ppm (parts per million) level. The ensemble of the peptide masses observed in a protein digest, such as a tryptic digest, may be used to search protein/DNA databases in a method called peptide mass fingerprinting. In this approach, protein entries in a database are ranked according to the number of experimental peptide masses that match the predicted trypsin digestion pattern. Commercially available software utilizes a search algorithm that provides a scoring scheme based on the size of the databases, the number of matching peptides, and the different peptides. Depending on the number of peptides observed, the accuracy of the measurement, and the size of the genome of the particular species, unambiguous protein identification may be obtained.

Statistical analysis may be performed upon each protein match to determine the validity of the match. Typical constraints include error tolerances within 0.1 Da for monoisotopic peptide masses, cysteines may be alkylated and searched as carboxyamidomethyl modifications, 0 or 1 missed enzyme cleavages, and no methionine oxidations allowed. Identified proteins may be stored automatically in a relational database with software links to SDS-PAGE images and ligand sequences. Often even a partial peptide map is specific enough for identification of the protein. If no protein match is found, a more error-tolerant search can be used, for example using fewer peptides or allowing a larger margin error with respect to mass accuracy.

Other mass spectroscopy methods such as tandem mass spectrometry or post source decay may be used to obtain sequence information about proteins that cannot be identified by peptide mass mapping, or to confirm the identity of proteins that are tentatively identified by an error-tolerant peptide mass search described above. (Griffin et al, Rapid Commun. Mass. Spectrom. 1995, 9, 1546-51).

(b) Analysis of Proteins by Nuclear Magnetic Resonance (NMR)

NMR may be used to characterize the structure of a polypeptide in accordance with the methods of the invention. In particular, NMR can be used, for example, to determine the three dimensional structure, the conformational state, the aggregation level, the state of protein folding/unfolding or the dynamic properties of a polypeptide. For example, the present invention contemplates a method for determining three dimensional structure information of a polypeptide of the invention, the method comprising: (a) generating a purified isotopically labeled polypeptide of the invention; and (b) subjecting the polypeptide to NMR spectroscopic analysis, thereby determining information about its three dimensional structure.

Interaction between a polypeptide and another molecule can also be monitored using NMR. Thus, the invention encompasses methods for detecting, designing and characterizing interactions between a polypeptide and another molecule, including polypeptides, nucleic acids and small molecules, utilizing NMR techniques. For example, the present invention contemplates a method for determining three dimensional structure information of a polypeptide of the invention, or a fragment thereof, while the polypeptide is complexed with another molecule, the method comprising: (a) generating a purified isotopically labeled polypeptide of the invention, or a fragment thereof; (b) forming a complex between the polypeptide and the other molecule; and (c) subjecting the complex to NMR spectroscopic analysis, thereby determining information about the three dimensional structure of the polypeptide. In another aspect, the present invention contemplates a method for identifying compounds that bind to a polypeptide of the invention, or a fragment thereof, the method comprising: (a) generating a first NMR spectrum of an isotopically labeled polypeptide of the invention, or a fragment thereof; (b) exposing the polypeptide to one or more chemical compounds; (c) generating a second NMR spectrum of the polypeptide which has been exposed to one or more chemical compounds; and (d) comparing the first and second spectra to determine differences between the first and the second spectra, wherein the differences are indicative of one or more compounds that have bound to the polypeptide.

Briefly, the NMR technique involves placing the material to be examined (usually in a suitable solvent) in a powerful magnetic field and irradiating it with radio frequency (rf) electromagnetic radiation. The nuclei of the various atoms will align themselves with the magnetic field until energized by the rf radiation. They then absorb this resonant energy and re-radiate it at a frequency dependent on i) the type of nucleus and ii) its atomic environment. Moreover, resonant energy may be passed from one nucleus to another, either through bonds or through three-dimensional space, thus giving information about the environment of a particular nucleus and nuclei in its vicinity.

However, it is important to recognize that not all nuclei are NMR active. Indeed, not all isotopes of the same element are active. For example, whereas “ordinary” hydrogen, ¹H, is NMR active, heavy hydrogen (deuterium), ²H, is not active in the same way. Thus, any material that normally contains ¹H hydrogen may be rendered “invisible” in the hydrogen NMR spectrum by replacing all or almost all the ¹H hydrogens with ²H. It is for this reason that NMR spectroscopic analyses of water-soluble materials frequently are performed in ²H₂O (or deuterium) to eliminate the water signal.

Conversely, “ordinary” carbon, ¹²C, is NMR inactive whereas the stable isotope, ¹³C, present to about 1% of total carbon in nature, is active. Similarly, while “ordinary” nitrogen, ¹⁴N, is NMR active, it has undesirable properties for NMR and resonates at a different frequency from the stable isotope ¹⁵N, present to about 0.4% of total nitrogen in nature.

By labeling proteins with ¹⁵N and ¹⁵N/¹³C, it is possible to conduct analytical NMR of macromolecules with weights of 15 kD and 40 kD, respectively. More recently, partial deuteration of the protein in addition to ¹³C- and ¹⁵N-labeling has increased the possible weight of proteins and protein complexes for NMR analysis still further, to approximately 60-70 kD. See Shan et al., J. Am. Chem. Soc., 118:6570-6579 (1996); L. E. Kay, Methods Enzymol., 339:174-203 (2001); and K. H. Gardner & L. E. Kay, Annu Rev Biophys Biomol Struct., 27:357-406 (1998); and references cited therein.

Isotopic substitution may be accomplished by growing a bacterium or yeast or other type of cultured cells, transformed by genetic engineering to produce the protein of choice, in a growth medium containing ¹³C-, ¹⁵N- and/or ²H-labeled substrates. In certain instances, bacterial growth media consists of ¹³C-labeled glucose and/or ¹⁵N-labeled ammonium salts dissolved in D₂O where necessary. Kay, L. et al., Science, 249:411 (1990) and references therein and Bax, A., J. Am. Chem. Soc., 115, 4369 (1993). More recently, isotopically labeled media especially adapted for the labeling of bacterially produced macromolecules have been described. See U.S. Pat. No. 5,324,658.

The goal of these methods has been to achieve universal and/or random isotopic enrichment of all of the amino acids of the protein. By contrast, other methods allow only certain residues to be relatively enriched in ¹H, ²H, ¹³C and ¹⁵N. For example, Kay et al., J. Mol. Biol., 263, 627-636 (1996) and Kay et al., J. Am. Chem. Soc., 119, 7599-7600 (1997) have described methods whereby isoleucine, alanine, valine and leucine residues in a protein may be labeled with ²H, ¹³C and ¹⁵N, and may be specifically labeled with ¹H at the terminal methyl position. In this way, study of the proton-proton interactions between some amino acids may be facilitated. Similarly, a cell-free system has been described by Yokoyama et al., J. Biomol. NMR, 6(2), 129-134 (1995), wherein a transcription-translation system derived from E. coli was used to express human Ha-Ras protein incorporating ¹⁵N into serine and/or aspartic acid.

Techniques for producing isotopically labeled proteins and macromolecules, such as glycoproteins, in mammalian or insect cells have been described. See U.S. Pat. Nos. 5,393,669 and 5,627,044; Weller, C. T., Biochem., 35, 8815-23 (1996) and Lustbader, J. W., J. Biomol. NMR, 7, 295-304 (1996). Other methods for producing polypeptides and other molecules with labels appropriate for NMR are known in the art.

The present invention contemplates using a variety of solvents which are appropriate for NMR. For ¹H NMR, a deuterium lock solvent may be used. Exemplary deuterium lock solvents include acetone (CD₃COCD₃), chloroform (CDCl₃), dichloro methane (CD₂Cl₂), methylnitrile (CD₃CN), benzene (C₆D₆), water (D₂O), diethylether ((CD₃CD₂)₂O), dimethylether ((CD₃)₂O), N,N-dimethylformamide ((CD₃)₂NCDO), dimethyl sulfoxide (CD₃SOCD₃), ethanol (CD₃CD₂OD), methanol (CD₃OD), tetrahydrofuran (C₄D₈O), toluene (C₆D₅CD₃), pyridine (C₅D₅N) and cyclohexane (C₆H₁₂). For example, the present invention contemplates a composition comprising a polypeptide of the invention and a deuterium lock solvent.

The 2-dimensional ¹H-¹⁵N HSQC (Heteronuclear Single Quantum Correlation) spectrum provides a diagnostic fingerprint of conformational state, aggregation level, state of protein folding, and dynamic properties of a polypeptide (Yee et al, PNAS 99, 1825-30 (2002)). Polypeptides in aqueous solution usually populate an ensemble of 3-dimensional structures which can be determined by NMR. When the polypeptide is a stable globular protein or domain of a protein, then the ensemble of solution structures is one of very closely related conformations. In this case, one peak is expected for each non-proline residue with a dispersion of resonance frequencies with roughly equal intensity. Additional pairs of peaks from side-chain NH₂ groups are also often observed, and correspond to the approximate number of Gln and Asn residues in the protein. This type of HSQC spectra usually indicates that the protein is amenable to structure determination by NMR methods.

If the HSQC spectrum shows well-dispersed peaks but there are either too few or too many in number, and/or the peak intensities differ throughout the spectrum, then the protein likely does not exist in a single globular conformation. Such spectral features are indicative of conformational heterogeneity with slow or nonexistent inter-conversion between states (too many peaks) or the presence of dynamic processes on an intermediate timescale that can broaden and obscure the NMR signals. Proteins with this type of spectrum can sometimes be stabilized into a single conformation by changing either the protein construct, the solution conditions, temperature or by binding of another molecule.

The ¹H-¹⁵N HSQC can also indicate whether a protein has formed large nonspecific aggregates or has dynamic properties. Alternatively, proteins that are largely unfolded, e.g., having very little regular secondary structure, result in ¹H-¹⁵N HSQC spectra in which the peaks are all very narrow and intense, but have very little spectral dispersion in the ¹⁵N-dimension. This reflects the fact that many or most of the amide groups of amino acids in unfolded polypeptides are solvent exposed and experience similar chemical environments resulting in similar ¹H chemical shifts.

The use of the ¹H-¹⁵N HSQC, can thus allow the rapid characterization of the conformational state, aggregation level, state of protein folding, and dynamic properties of a polypeptide. Additionally, other 2D spectra such as ¹H-¹³C HSQC, or HNCO spectra can also be used in a similar manner. Further use of the ¹H-¹⁵N HSQC combined with relaxation measurements can reveal the molecular rotational correlation time and dynamic properties of polypeptides. The rotational correlation time is proportional to size of the protein and therefore can reveal if it forms specific homo-oligomers such as homodimers, homotetramers, etc.

The structure of stable globular proteins can be determined through a series of well-described procedures. For a general review of structure determination of globular proteins in solution by NMR spectroscopy, see Wüthrich, Science 243: 45-50 (1989). See also, Billeter et al., J. Mol. Biol. 155: 321-346 (1982). Current methods for structure determination usually require the complete or nearly complete sequence-specific assignment of ¹H-resonance frequencies of the protein and subsequent identification of approximate inter-hydrogen distances (from nuclear Overhauser effect (NOE) spectra) for use in restrained molecular dynamics calculations of the protein conformation. One approach for the analysis of NMR resonance assignments was first outlined by Wüthrich, Wagner and co-workers (Wüthrich, “NMR or proteins and nucleic acids” Wiley, New York, N.Y. (1986); Wüthrich, Science 243: 45-50 (1989); Billeter et al., J. Mol. Biol. 155: 321-346 (1982)). Newer methods for determining the structures of globular proteins include the use of residual dipolar coupling restraints (Tian et al., J Am Chem Soc. 2001 Nov. 28; 123(47):11791-6; Bax et al, Methods Enzymol. 2001; 339:127-74) and empirically derived conformational restraints (Zweckstetter & Bax, J Am Chem Soc. 2001 Sep. 26; 123(38):9490-1). It has also been shown that it may be possible to determine structures of globular proteins using only un-assigned NOE measurements. NMR may also be used to determine ensembles of many inter-converting, unfolded conformations (Choy and Forman-Kay, J Mol Biol. 2001 May 18; 308(5):1011-32).

NMR analysis of a polypeptide in the presence and absence of a test compound (e.g., a polypeptide, nucleic acid or small molecule) may be used to characterize interactions between a polypeptide and another molecule. Because the ¹H-¹⁵N HSQC spectrum and other simple 2D NMR experiments can be obtained very quickly (on the order of minutes depending on protein concentration and NMR instrumentation), they are very useful for rapidly testing whether a polypeptide is able to bind to another molecule. Changes in the resonance frequency (in one or both dimensions) of one or more peaks in the HSQC spectrum indicate an interaction with another molecule. Often only a subset of the peaks will have changes in resonance frequency upon binding to anther molecule, allowing one to map onto the structure those residues directly involved in the interaction or involved in conformational changes as a result of the interaction. If the interacting molecule is relatively large (protein or nucleic acid) the peak widths will also broaden due to the increased rotational correlation time of the complex. In some cases the peaks involved in the interaction may actually disappear from the NMR spectrum if the interacting molecule is in intermediate exchange on the NMR timescale (i.e., exchanging on and off the polypeptide at a frequency that is similar to the resonance frequency of the monitored nuclei).

To facilitate the acquisition of NMR data on a large number of compounds (e.g., a library of synthetic or naturally-occurring small organic compounds), a sample changer may be employed. Using the sample changer, a larger number of samples, numbering 60 or more, may be run unattended. To facilitate processing of the NMR data, computer programs are used to transfer and automatically process the multiple one-dimensional NMR data.

In one embodiment, the invention provides a screening method for identifying small molecules capable of interacting with a polypeptide of the invention. In one example, the screening process begins with the generation or acquisition of either a T₂-filtered or a diffusion-filtered one-dimensional proton spectrum of the compound or mixture of compounds. Means for generating T₂-filtered or diffusion-filtered one-dimensional proton spectra are well known in the art (see, e.g., S. Meiboom and D. Gill, Rev. Sci. Instrum. 29:688(1958), S. J. Gibbs and C. S. Johnson, Jr. J. Main. Reson. 93:395-402 (1991) and A. S. Altieri, et al. J. Am. Chem. Soc. 117: 7566-7567 (1995)).

Following acquisition of the first spectrum for the molecules, the ¹⁵N— or ¹³C-labeled polypeptide is exposed to one or more molecules. Where more than one test compound is to be tested simultaneously, it is preferred to use a library of compounds such as a plurality of small molecules. Such molecules are typically dissolved in perdeuterated dimethylsulfoxide. The compounds in the library may be purchased from vendors or created according to desired needs.

Individual compounds may be selected inter alia on the basis of size and molecular diversity for maximizing the possibility of discovering compounds that interact with widely diverse binding sites of a subject amino acid sequence or other polypeptides of the invention.

The NMR screening process of the present invention utilizes a range of test compound concentrations, e.g., from about 0.05 to about 1.0 mM. At those exemplary concentrations, compounds which are acidic or basic may significantly change the pH of buffered protein solutions. Chemical shifts are sensitive to pH changes as well as direct binding interactions, and false-positive chemical shift changes, which are not the result of test compound binding but of changes in pH, may therefore be observed. It may therefore be necessary to ensure that the pH of the buffered solution does not change upon addition of the test compound.

Following exposure of the test compounds to a polypeptide (e.g., the target molecule for the experiment) a second one-dimensional T₂- or diffusion-filtered spectrum is generated. For the T₂-filtered approach, that second spectrum is generated in the same manner as set forth above. The first and second spectra are then compared to determine whether there are any differences between the two spectra. Differences in the one-dimensional T₂-filtered spectra indicate that the compound is binding to, or otherwise interacting with, the target molecule. Those differences are determined using standard procedures well known in the art. For the diffusion-filtered method, the second spectrum is generated by looking at the spectral differences between low and high gradient strengths-thus selecting for those compounds whose diffusion rates are comparable to that observed in the absence of target molecule.

To discover additional molecules that bind to the protein, molecules are selected for testing based on the structure/activity relationships from the initial screen and/or structural information on the initial leads when bound to the protein. By way of example, the initial screening may result in the identification of compounds, all of which contain an aromatic ring. The second round of screening would then use other aromatic molecules as the test compounds.

In another embodiment, the methods of the invention utilize a process for detecting the binding of one ligand to a polypeptide in the presence of a second ligand. In accordance with this embodiment, a polypeptide is bound to the second ligand before exposing the polypeptide to the test compounds.

For more information on NMR methods encompassed by the present invention, see also: U.S. Pat. Nos. 5,668,734; 6,194,179; 6,162,627; 6,043,024; 5,817,474; 5,891,642, 5,989,827; 5,891,643; 6,077,682; WO 00/05414; WO 99/22019; Cavanagh, et al., Protein NMR Spectroscopy, Principles and Practice, 1996, Academic Press; Clore, et al., NMR of Proteins. In Topics in Molecular and Structural Biology, 1993, S. Neidle, Fuller, W., and Cohen, J. S., eds., Macmillan Press, Ltd., London; and Christendat et al., Nature Structural Biology 7: 903-909 (2000).

(c) Analysis of Proteins by X-Ray Crystallography

(i) X-Ray Structure Determination

Exemplary methods for obtaining the three dimensional structure of the crystalline form of a molecule or complex are described herein and, in view of this specification, variations on these methods will be apparent to those skilled in the art (see Ducruix and Geige 1992, IRL Press, Oxford, England).

A variety of methods involving x-ray crystallography are contemplated by the present invention. For example, the present invention contemplates producing a crystallized polypeptide of the invention, or a fragment thereof, by: (a) introducing into a host cell an expression vector comprising a nucleic acid encoding for a polypeptide of the invention, or a fragment thereof; (b) culturing the host cell in a cell culture medium to express the polypeptide or fragment; (c) isolating the polypeptide or fragment from the cell culture; and (d) crystallizing the polypeptide or fragment thereof. Alternatively, the present invention contemplates determining the three dimensional structure of a crystallized polypeptide of the invention, or a fragment thereof, by: (a) crystallizing a polypeptide of the invention, or a fragment thereof, such that the crystals will diffract x-rays to a resolution of 3.5 Å or better; and (b) analyzing the polypeptide or fragment by x-ray diffraction to determine the three-dimensional structure of the crystallized polypeptide.

X-ray crystallography techniques generally require that the protein molecules be available in the form of a crystal. Crystals may be grown from a solution containing a purified polypeptide of the invention, or a fragment thereof (e.g., a stable domain), by a variety of conventional processes. These processes include, for example, batch, liquid, bridge, dialysis, vapour diffusion (e.g., hanging drop or sitting drop methods). (See for example, McPherson, 1982 John Wiley, New York; McPherson, 1990, Eur. J. Biochem. 189: 1-23; Webber. 1991, Adv. Protein Chem. 41:1-36).

In certain embodiments, native crystals of the invention may be grown by adding precipitants to the concentrated solution of the polypeptide. The precipitants are added at a concentration just below that necessary to precipitate the protein. Water may be removed by controlled evaporation to produce precipitating conditions, which are maintained until crystal growth ceases.

The formation of crystals is dependent on a number of different parameters, including pH, temperature, protein concentration, the nature of the solvent and precipitant, as well as the presence of added ions or ligands to the protein. In addition, the sequence of the polypeptide being crystallized will have a significant affect on the success of obtaining crystals. Many routine crystallization experiments may be needed to screen all these parameters for the few combinations that might give crystal suitable for x-ray diffraction analysis (See, for example, Jancarik, J & Kim, S. H., J. Appl. Cryst. 1991 24: 409-411).

Crystallization robots may automate and speed up the work of reproducibly setting up large number of crystallization experiments. Once some suitable set of conditions for growing the crystal are found, variations of the condition may be systematically screened in order to find the set of conditions which allows the growth of sufficiently large, single, well ordered crystals. In certain instances, a polypeptide of the invention is co-crystallized with a compound that stabilizes the polypeptide.

A number of methods are available to produce suitable radiation for x-ray diffraction. For example, x-ray beams may be produced by synchrotron rings where electrons (or positrons) are accelerated through an electromagnetic field while traveling at close to the speed of light. Because the admitted wavelength may also be controlled, synchrotrons may be used as a tunable x-ray source Hendrickson W A., Trends Biochem Sci 2000 December; 25(12):637-43). For less conventional Laue diffraction studies, polychromatic x-rays covering a broad wavelength window are used to observe many diffraction intensities simultaneously (Stoddard, B. L., Curr. Opin. Struct Biol 1998 October; 8(5):612-8). Neutrons may also be used for solving protein crystal structures (Gutberlet T, Heinemann U & Steiner M., Acta Crystallogr D 2001; 57: 349-54).

Before data collection commences, a protein crystal may be frozen to protect it from radiation damage. A number of different cryo-protectants may be used to assist in freezing the crystal, such as methyl pentanediol (MPD), isopropanol, ethylene glycol, glycerol, formate, citrate, mineral oil, or a low-molecular-weight polyethylene glycol (PEG). The present invention contemplates a composition comprising a polypeptide of the invention and a cryo-protectant. As an alternative to freezing the crystal, the crystal may also be used for diffraction experiments performed at temperatures above the freezing point of the solution. In these instances, the crystal may be protected from drying out by placing it in a narrow capillary of a suitable material (generally glass or quartz) with some of the crystal growth solution included in order to maintain vapour pressure.

X-ray diffraction results may be recorded by a number of ways know to one of skill in the art. Examples of area electronic detectors include charge coupled device detectors, multi-wire area detectors and phosphoimager detectors (Amemiya, Y, 1997. Methods in Enzymology, Vol. 276. Academic Press, San, Diego, pp. 233-243; Westbrook, E. M., Naday, I. 1997. Methods in Enzymology, Vol. 276. Academic Press, San Diego, pp. 244-268; 1997. Kahn, R. & Fourme, R. Methods in Enzymology, Vol. 276. Academic Press, San Diego, pp. 268-286).

A suitable system for laboratory data collection might include a Bruker AXS Proteum R system, equipped with a copper rotating anode source, Confocal Max-Flux™ optics and a SMART 6000 charge coupled device detector. Collection of x-ray diffraction patterns are well documented by those skilled in the art (See, for example, Ducruix and Geige, 1992, IRL Press, Oxford, England).

The theory behind diffraction by a crystal upon exposure to x-rays is well known. Because phase information is not directly measured in the diffraction experiment, and is needed to reconstruct the electron density map, methods that can recover this missing information are required. One method of solving structures ab initio are the real/reciprocal space cycling techniques. Suitable real/reciprocal space cycling search programs include shake-and-bake (Weeks C M, DeTitta G T, Hauptman H A, Thuman P, Miller R Acta Crystallogr A 1994; V50: 210-20).

Other methods for deriving phases may also be needed. These techniques generally rely on the idea that if two or more measurements of the same reflection are made where strong, measurable, differences are attributable to the characteristics of a small subset of the atoms alone, then the contributions of other atoms can be, to a first approximation, ignored, and positions of these atoms may be determined from the difference in scattering by one of the above techniques. Knowing the position and scattering characteristics of those atoms, one may calculate what phase the overall scattering must have had to produce the observed differences.

One version of this technique is isomorphous replacement technique, which requires the introduction of new, well ordered, x-ray scatterers into the crystal. These additions are usually heavy metal atoms, (so that they make a significant difference in the diffraction pattern); and if the additions do not change the structure of the molecule or of the crystal cell, the resulting crystals should be isomorphous. Isomorphous replacement experiments are usually performed by diffusing different heavy-metal metals into the channels of a pre-existing protein crystal. Growing the crystal from protein that has been soaked in the heavy atom is also possible (Petsko, G. A., 1985. Methods in Enzymology, Vol. 114. Academic Press, Orlando, pp. 147-156). Alternatively, the heavy atom may also be reactive and attached covalently to exposed amino acid side chains (such as the sulfur atom of cysteine) or it may be associated through non-covalent interactions. It is sometimes possible to replace endogenous light metals in metallo-proteins with heavier ones, e.g., zinc by mercury, or calcium by samarium (Petsko, G. A., 1985. Methods in Enzymology, Vol. 114. Academic Press, Orlando, pp. 147-156). Exemplary sources for such heavy compounds include, without limitation, sodium bromide, sodium selenate, trimethyl lead acetate, mercuric chloride, methyl mercury acetate, platinum tetracyanide, platinum tetrachloride, nickel chloride, and europium chloride.

A second technique for generating differences in scattering involves the phenomenon of anomalous scattering. X-rays that cause the displacement of an electron in an inner shell to a higher shell are subsequently rescattered, but there is a time lag that shows up as a phase delay. This phase delay is observed as a (generally quite small) difference in intensity between reflections known as Friedel mates that would be identical if no anomalous scattering were present. A second effect related to this phenomenon is that differences in the intensity of scattering of a given atom will vary in a wavelength dependent manner, given rise to what are known as dispersive differences. In principle anomalous scattering occurs with all atoms, but the effect is strongest in heavy atoms, and may be maximized by using x-rays at a wavelength where the energy is equal to the difference in energy between shells. The technique therefore requires the incorporation of some heavy atom much as is needed for isomorphous replacement, although for anomalous scattering a wider variety of atoms are suitable, including lighter metal atoms (copper, zinc, iron) in metallo-proteins. One method for preparing a protein for anomalous scattering involves replacing the methionine residues in whole or in part with selenium containing seleno-methionine. Soaks with halide salts such as bromides and other non-reactive ions may also be effective (Dauter Z, Li M, Wlodawer A., Acta Crystallogr D 2001; 57: 239-49).

In another process, known as multiple anomalous scattering or MAD, two to four suitable wavelengths of data are collected. (Hendrickson, W. A. and Ogata, C. M. 1997 Methods in Enzymology 276, 494-523). Phasing by various combinations of single and multiple isomorphous and anomalous scattering are possible too. For example, SIRAS (single isomorphous replacement with anomalous scattering) utilizes both the isomorphous and anomalous differences for one derivative to derive phases. More traditionally, several different heavy atoms are soaked into different crystals to get sufficient phase information from isomorphous differences while ignoring anomalous scattering, in the technique known as multiple isomorphous replacement (MIR) (Petsko, G. A., 1985. Methods in Enzymology, Vol. 114. Academic Press, Orlando, pp. 147-156).

Additional restraints on the phases may be derived from density modification techniques. These techniques use either generally known features of electron density distribution or known facts about that particular crystal to improve the phases. For example, because protein regions of the crystal scatter more strongly than solvent regions, solvent flattening/flipping may be used to adjust phases to make solvent density a uniform flat value (Zhang, K. Y. J., Cowtan, K and Main, P. Methods in Enzymology 277, 1997 Academic Press, Orlando pp 53-64). If more than one molecule of the protein is present in the asymmetric unit, the fact that the different molecules should be virtually identical may be exploited to further reduce phase error using non-crystallographic symmetry averaging (Villieux, F. M. D. and Read, R. J. Methods in Enzymology 277, 1997 Academic Press, Orlando pp 18-52). Suitable programs for performing these processes include DM and other programs of the CCP4 suite (Collaborative Computational Project, Number 4. 1994. Acta Cryst. D50, 760-763) and CNX.

The unit cell dimensions, symmetry, vector amplitude and derived phase information can be used in a Fourier transform function to calculate the electron density in the unit cell, i.e., to generate an experimental electron density map. This may be accomplished using programs of the CNX or CCP4 packages. The resolution is measured in Ångstrom (Å) units, and is closely related to how far apart two objects need to be before they can be reliably distinguished. The smaller this number is, the higher the resolution and therefore the greater the amount of detail that can be seen. Preferably, crystals of the invention diffract x-rays to a resolution of better than about 4.0, 3.5, 3.0, 2.5, 2.0, 1.5, 1.0, 0.5 Å or better.

As used herein, the term “modeling” includes the quantitative and qualitative analysis of molecular structure and/or function based on atomic structural information and interaction models. The term “modeling” includes conventional numeric-based molecular dynamic and energy minimization models, interactive computer graphic models, modified molecular mechanics models, distance geometry and other structure-based constraint models.

Model building may be accomplished by either the crystallographer using a computer graphics program such as TURBO or 0 (Jones, T A. et al., Acta Crystallogr. A47, 100-119, 1991) or, under suitable circumstances, by using a fully automated model building program, such as wARP (Anastassis Perrakis, Richard Morris & Victor S. Lamzin; Nature Structural Biology, May 1999 Volume 6 Number 5 pp 458-463) or MAID (Levitt, D. G., Acta Crystallogr. D 2001 V57: 1013-9). This structure may be used to calculate model-derived diffraction amplitudes and phases. The model-derived and experimental diffraction amplitudes may be compared and the agreement between them can be described by a parameter referred to as R-factor. A high degree of correlation in the amplitudes corresponds to a low R-factor value, with 0.0 representing exact agreement and 0.59 representing a completely random structure. Because the R-factor may be lowered by introducing more free parameters into the model, an unbiased, cross-correlated version of the R-factor known as the R-free gives a more objective measure of model quality. For the calculation of this parameter a subset of reflections (generally around 10%) are set aside at the beginning of the refinement and not used as part of the refinement target. These reflections are then compared to those predicted by the model (Kleywegt G J, Brunger A T, Structure 1996 Aug. 15; 4(8):897-904).

The model may be improved using computer programs that maximize the probability that the observed data was produced from the predicted model, while simultaneously optimizing the model geometry. For example, the CNX program may be used for model refinement, as can the XPLOR program (1992, Nature 355:472-475, G. N. Murshudov, A. A. Vagin and E. J. Dodson, (1997) Acta Cryst. D 53, 240-255). In order to maximize the convergence radius of refinement, simulated annealing refinement using torsion angle dynamics may be employed in order to reduce the degrees of freedom of motion of the model (Adams P D, Pannu N S, Read R J, Brunger A T., Proc Natl Acad Sci USA 1997 May 13; 94(10):5018-23). Where experimental phase information is available (e.g. where MAD data was collected) Hendrickson-Lattman phase probability targets may be employed. Isotropic or anisotropic domain, group or individual temperature factor refinement, may be used to model variance of the atomic position from its mean. Well defined peaks of electron density not attributable to protein atoms are generally modeled as water molecules. Water molecules may be found by manual inspection of electron density maps, or with automatic water picking routines. Additional small molecules, including ions, cofactors, buffer molecules or substrates may be included in the model if sufficiently unambiguous electron density is observed in a map.

In general, the R-free is rarely as low as 0.15 and may be as high as 0.35 or greater for a reasonably well-determined protein structure. The residual difference is a consequence of approximations in the model (inadequate modeling of residual structure in the solvent, modeling atoms as isotropic Gaussian spheres, assuming all molecules are identical rather than having a set of discrete conformers, etc.) and errors in the data (Lattman E E., Proteins 1996; 25: i-ii). In refined structures at high resolution, there are usually no major errors in the orientation of individual residues, and the estimated errors in atomic positions are usually around 0.1-0.2 up to 0.3 Å.

The three dimensional structure of a new crystal may be modeled using molecular replacement. The term “molecular replacement” refers to a method that involves generating a preliminary model of a molecule or complex whose structure coordinates are unknown, by orienting and positioning a molecule whose structure coordinates are known within the unit cell of the unknown crystal, so as best to account for the observed diffraction pattern of the unknown crystal. Phases may then be calculated from this model and combined with the observed amplitudes to give an approximate Fourier synthesis of the structure whose coordinates are unknown. This, in turn, can be subject to any of the several forms of refinement to provide a final, accurate structure of the unknown crystal. Lattman, E., “Use of the Rotation and Translation Functions”, in Methods in Enzymology, 115, pp. 55-77 (1985); M. G. Rossmann, ed., “The Molecular Replacement Method”, Int. Sci. Rev. Ser., No. 13, Gordon & Breach, New York, (1972).

Commonly used computer software packages for molecular replacement are CNX, X-PLOR (Brunger 1992, Nature 355: 472-475), AMoRE (Navaza, 1994, Acta Crystallogr. A50:157-163), the CCP4 package, the MERLOT package (P. M. D. Fitzgerald, J. Appl. Cryst., Vol. 21, pp. 273-278, 1988) and XTALVIEW (McCree et al (1992) J. Mol. Graphics 10: 44-46). The quality of the model may be analyzed using a program such as PROCHECK or 3D-Profiler (Laskowski et al 1993 J. Appl. Cryst. 26:283-291; Luthy R. et al, Nature. 356: 83-85, 1992; and Bowie, J. U. et al, Science 253: 164-170, 1991).

Homology modeling (also known as comparative modeling or knowledge-based modeling) methods may also be used to develop a three dimensional model from a polypeptide sequence based on the structures of known proteins. The method utilizes a computer model of a known protein, a computer representation of the amino acid sequence of the polypeptide with an unknown structure, and standard computer representations of the structures of amino acids. This method is well known to those skilled in the art (Greer, 1985, Science 228, 1055; Bundell et al 1988, Eur. J. Biochem. 172, 513; Knighton et al., 1992, Science 258:130-135, http://biochem.vt.edulcourses/-modeling/homology.htn). Computer programs that can be used in homology modeling are QUANTA and the Homology module in the Insight II modeling package distributed by Molecular Simulations Inc, or MODELLER Rockefeller University, www.iucr.ac.uk/sinris-top/logical/prg-modeller.html).

Once a homology model has been generated it is analyzed to determine its correctness. A computer program available to assist in this analysis is the Protein Health module in QUANTA which provides a variety of tests. Other programs that provide structure analysis along with output include PROCHECK and 3D-Profiler (Luthy R. et al, Nature 356: 83-85, 1992; and Bowie, J. U. et al, Science 253: 164-170, 1991). Once any irregularities have been resolved, the entire structure may be further refined.

Other molecular modeling techniques may also be employed in accordance with this invention. See, e.g., Cohen, N. C. et al, J. Med. Chem., 33, pp. 883-894 (1990). See also, Navix, M. A. and M. A. Marko, Current Opinions in Structural Biology, 2, pp. 202-210 (1992).

Under suitable circumstances, the entire process of solving a crystal structure may be accomplished in an automated fashion by a system such as ELVES (http://ucxray.berkeley.edu/˜jamesh/elves/index.html) with little or no user intervention.

(ii) X-Ray Structure

The present invention provides methods for determining some or all of the structural coordinates for amino acids of a polypeptide of the invention, or a complex thereof.

In another aspect, the present invention provides methods for identifying a druggable region of a polypeptide of the invention. For example, one such method includes: (a) obtaining crystals of a polypeptide of the invention or a fragment thereof such that the three dimensional structure of the crystallized protein can be determined to a resolution of 3.5 Å or better; (b) determining the three dimensional structure of the crystallized polypeptide or fragment using x-ray diffraction; and (c) identifying a druggable region of a polypeptide of the invention based on the three-dimensional structure of the polypeptide or fragment.

A three dimensional structure of a molecule or complex may be described by the set of atoms that best predict the observed diffraction data (that is, which possesses a minimal R value). Files may be created for the structure that defines each atom by its chemical identity, spatial coordinates in three dimensions, root mean squared deviation from the mean observed position and fractional occupancy of the observed position.

Those of skill in the art understand that a set of structure coordinates for an protein, complex or a portion thereof, is a relative set of points that define a shape in three dimensions. Thus, it is possible that an entirely different set of coordinates could define a similar or identical shape. Moreover, slight variations in the individual coordinates may have little affect on overall shape. Such variations in coordinates may be generated because of mathematical manipulations of the structure coordinates. For example, structure coordinates could be manipulated by crystallographic permutations of the structure coordinates, fractionalization of the structure coordinates, integer additions or subtractions to sets of the structure coordinates, inversion of the structure coordinates or any combination of the above. Alternatively, modifications in the crystal structure due to mutations, additions, substitutions, and/or deletions of amino acids, or other changes in any of the components that make up the crystal, could also yield variations in structure coordinates. Such slight variations in the individual coordinates will have little affect on overall shape. If such variations are within an acceptable standard error as compared to the original coordinates, the resulting three-dimensional shape is considered to be structurally equivalent. It should be noted that slight variations in individual structure coordinates of a polypeptide of the invention or a complex thereof would not be expected to significantly alter the nature of modulators that could associate with a druggable region thereof. Thus, for example, a modulator that bound to the active site of a polypeptide of the invention would also be expected to bind to or interfere with another active site whose structure coordinates define a shape that falls within the acceptable error.

A crystal structure of the present invention may be used to make a structural or computer model of the polypeptide, complex or portion thereof. A model may represent the secondary, tertiary and/or quaternary structure of the polypeptide, complex or portion. The configurations of points in space derived from structure coordinates according to the invention can be visualized as, for example, a holographic image, a stereodiagram, a model or a computer-displayed image, and the invention thus includes such images, diagrams or models.

(iii) Structural Equivalents

Various computational analyses can be used to determine whether a molecule or the active site portion thereof is structurally equivalent with respect to its three-dimensional structure, to all or part of a structure of a polypeptide of the invention or a portion thereof.

For the purpose of this invention, any molecule or complex or portion thereof, that has a root mean square deviation of conserved residue backbone atoms (N, Cα, C, O) of less than about 1.75 Å, when superimposed on the relevant backbone atoms described by the reference structure coordinates of a polypeptide of the invention, is considered “structurally equivalent” to the reference molecule. That is to say, the crystal structures of those portions of the two molecules are substantially identical, within acceptable error. Alternatively, the root mean square deviation may be is less than about 1.50, 1.40, 1.25, 1.0, 0.75, 0.5 or 0.35 Å.

The term “root mean square deviation” is understood in the art and means the square root of the arithmetic mean of the squares of the deviations. It is a way to express the deviation or variation from a trend or object.

In another aspect, the present invention provides a scalable three-dimensional configuration of points, at least a portion of said points, and preferably all of said points, derived from structural coordinates of at least a portion of a polypeptide of the invention and having a root mean square deviation from the structure coordinates of the polypeptide of the invention of less than 1.50, 1.40, 1.25, 1.0, 0.75, 0.5 or 0.35 Å. In certain embodiments, the portion of a polypeptide of the invention is 25%, 33%, 50%, 66%, 75%, 85%, 90% or 95% or more of the amino acid residues contained in the polypeptide.

In another aspect, the present invention provides a molecule or complex including a druggable region of a polypeptide of the invention, the druggable region being defined by a set of points having a root mean square deviation of less than about 1.75 Å from the structural coordinates for points representing (a) the backbone atoms of the amino acids contained in a druggable region of a polypeptide of the invention, (b) the side chain atoms (and optionally the Cα atoms) of the amino acids contained in such druggable region, or (c) all the atoms of the amino acids contained in such druggable region. In certain embodiments, only a portion of the amino acids of a druggable region may be included in the set of points, such as 25%, 33%, 50%, 66%, 75%, 85%, 90% or 95% or more of the amino acid residues contained in the druggable region. In certain embodiments, the root mean square deviation may be less than 1.50, 1.40, 1.25, 1.0, 0.75, 0.5, or 0.35 Å. In still other embodiments, instead of a druggable region, a stable domain, fragment or structural motif is used in place of a druggable region.

(iv) Machine Displays and Machine Readable Storage Media

The invention provides a machine-readable storage medium including a data storage material encoded with machine readable data which, when using a machine programmed with instructions for using said data, displays a graphical three-dimensional representation of any of the molecules or complexes, or portions thereof, of this invention. In another embodiment, the graphical three-dimensional representation of such molecule, complex or portion thereof includes the root mean square deviation of certain atoms of such molecule by a specified amount, such as the backbone atoms by less than 0.8 Å. In another embodiment, a structural equivalent of such molecule, complex, or portion thereof, may be displayed. In another embodiment, the portion may include a druggable region of the polypeptide of the invention.

According to one embodiment, the invention provides a computer for determining at least a portion of the structure coordinates corresponding to x-ray diffraction data obtained from a molecule or complex, wherein said computer includes: (a) a machine-readable data storage medium comprising a data storage material encoded with machine-readable data, wherein said data comprises at least a portion of the structural coordinates of a polypeptide of the invention; (b) a machine-readable data storage medium comprising a data storage material encoded with machine-readable data, wherein said data comprises x-ray diffraction data from said molecule or complex; (c) a working memory for storing instructions for processing said machine-readable data of (a) and (b); (d) a central-processing unit coupled to said working memory and to said machine-readable data storage medium of (a) and (b) for performing a Fourier transform of the machine readable data of (a) and for processing said machine readable data of (b) into structure coordinates; and (e) a display coupled to said central-processing unit for displaying said structure coordinates of said molecule or complex. In certain embodiments, the structural coordinates displayed are structurally equivalent to the structural coordinates of a polypeptide of the invention.

In an alternative embodiment, the machine-readable data storage medium includes a data storage material encoded with a first set of machine readable data which includes the Fourier transform of the structure coordinates of a polypeptide of the invention or a portion thereof, and which, when using a machine programmed with instructions for using said data, can be combined with a second set of machine readable data including the x-ray diffraction pattern of a molecule or complex to determine at least a portion of the structure coordinates corresponding to the second set of machine readable data.

For example, a system for reading a data storage medium may include a computer including a central processing unit (“CPU”), a working memory which may be, e.g., RAM (random access memory) or “core” memory, mass storage memory (such as one or more disk drives or CD-ROM drives), one or more display devices (e.g., cathode-ray tube (“CRT”) displays, light emitting diode (“LED”) displays, liquid crystal displays (“LCDs”), electroluminescent displays, vacuum fluorescent displays, field emission displays (“FEDs”), plasma displays, projection panels, etc.), one or more user input devices (e.g., keyboards, microphones, mice, touch screens, etc.), one or more input lines, and one or more output lines, all of which are interconnected by a conventional bidirectional system bus. The system may be a stand-alone computer, or may be networked (e.g., through local area networks, wide area networks, intranets, extranets, or the internet) to other systems (e.g., computers, hosts, servers, etc.). The system may also include additional computer controlled devices such as consumer electronics and appliances.

Input hardware may be coupled to the computer by input lines and may be implemented in a variety of ways. Machine-readable data of this invention may be inputted via the use of a modem or modems connected by a telephone line or dedicated data line. Alternatively or additionally, the input hardware may include CD-ROM drives or disk drives. In conjunction with a display terminal, a keyboard may also be used as an input device.

Output hardware may be coupled to the computer by output lines and may similarly be implemented by conventional devices. By way of example, the output hardware may include a display device for displaying a graphical representation of an active site of this invention using a program such as QUANTA as described herein. Output hardware might also include a printer, so that hard copy output may be produced, or a disk drive, to store system output for later use.

In operation, a CPU coordinates the use of the various input and output devices, coordinates data accesses from mass storage devices, accesses to and from working memory, and determines the sequence of data processing steps. A number of programs may be used to process the machine-readable data of this invention. Such programs are discussed in reference to the computational methods of drug discovery as described herein. References to components of the hardware system are included as appropriate throughout the following description of the data storage medium.

Machine-readable storage devices useful in the present invention include, but are not limited to, magnetic devices, electrical devices, optical devices, and combinations thereof. Examples of such data storage devices include, but are not limited to, hard disk devices, CD devices, digital video disk devices, floppy disk devices, removable hard disk devices, magneto-optic disk devices, magnetic tape devices, flash memory devices, bubble memory devices, holographic storage devices, and any other mass storage peripheral device. It should be understood that these storage devices include necessary hardware (e.g., drives, controllers, power supplies, etc.) as well as any necessary media (e.g., disks, flash cards, etc.) to enable the storage of data.

In one embodiment, the present invention contemplates a computer readable storage medium comprising structural data, wherein the data include the identity and three-dimensional coordinates of a polypeptide of the invention or portion thereof. In another aspect, the present invention contemplates a database comprising the identity and three-dimensional coordinates of a polypeptide of the invention or a portion thereof. Alternatively, the present invention contemplates a database comprising a portion or all of the atomic coordinates of a polypeptide of the invention or portion thereof.

(v) Structurally Similar Molecules and Complexes

Structural coordinates for a polypeptide of the invention can be used to aid in obtaining structural information about another molecule or complex. This method of the invention allows determination of at least a portion of the three-dimensional structure of molecules or molecular complexes which contain one or more structural features that are similar to structural features of a polypeptide of the invention. Similar structural features can include, for example, regions of amino acid identity, conserved active site or binding site motifs, and similarly arranged secondary structural elements (e.g., α helices and β sheets). Many of the methods described above for determining the structure of a polypeptide of the invention may be used for this purpose as well.

For the present invention, a “structural homolog” is a polypeptide that contains one or more amino acid substitutions, deletions, additions, or rearrangements with respect to a subject amino acid sequence or other polypeptide of the invention, but that, when folded into its native conformation, exhibits or is reasonably expected to exhibit at least a portion of the tertiary (three-dimensional) structure of the polypeptide encoded by the related subject amino acid sequence or such other polypeptide of the invention. For example, structurally homologous molecules can contain deletions or additions of one or more contiguous or noncontiguous amino acids, such as a loop or a domain. Structurally homologous molecules also include modified polypeptide molecules that have been chemically or enzymatically derivatized at one or more constituent amino acids, including side chain modifications, backbone modifications, and N- and C-terminal modifications including acetylation, hydroxylation, methylation, amidation, and the attachment of carbohydrate or lipid moieties, cofactors, and the like.

By using molecular replacement, all or part of the structure coordinates of a polypeptide of the invention can be used to determine the structure of a crystallized molecule or complex whose structure is unknown more quickly and efficiently than attempting to determine such information ab initio. For example, in one embodiment this invention provides a method of utilizing molecular replacement to obtain structural information about a molecule or complex whose structure is unknown including: (a) crystallizing the molecule or complex of unknown structure; (b) generating an x-ray diffraction pattern from said crystallized molecule or complex; and (c) applying at least a portion of the structure coordinates for a polypeptide of the invention to the x-ray diffraction pattern to generate a three-dimensional electron density map of the molecule or complex whose structure is unknown.

In another aspect, the present invention provides a method for generating a preliminary model of a molecule or complex whose structure coordinates are unknown, by orienting and positioning the relevant portion of a polypeptide of the invention within the unit cell of the crystal of the unknown molecule or complex so as best to account for the observed x-ray diffraction pattern of the crystal of the molecule or complex whose structure is unknown.

Structural information about a portion of any crystallized molecule or complex that is sufficiently structurally similar to a portion of a polypeptide of the invention may be resolved by this method. In addition to a molecule that shares one or more structural features with a polypeptide of the invention, a molecule that has similar bioactivity, such as the same catalytic activity, substrate specificity or ligand binding activity as a polypeptide of the invention, may also be sufficiently structurally similar to a polypeptide of the invention to permit use of the structure coordinates for a polypeptide of the invention to solve its crystal structure.

In another aspect, the method of molecular replacement is utilized to obtain structural information about a complex containing a polypeptide of the invention, such as a complex between a modulator and a polypeptide of the invention (or a domain, fragment, ortholog, homolog etc. thereof). In certain instances, the complex includes a polypeptide of the invention (or a domain, fragment, ortholog, homolog etc. thereof) co-complexed with a modulator. For example, in one embodiment, the present invention contemplates a method for making a crystallized complex comprising a polypeptide of the invention, or a fragment thereof, and a compound having a molecular weight of less than 5 kDa, the method comprising: (a) crystallizing a polypeptide of the invention such that the crystals will diffract x-rays to a resolution of 3.5 Å or better; and (b) soaking the crystal in a solution comprising the compound having a molecular weight of less than 5 kDa, thereby producing a crystallized complex comprising the polypeptide and the compound.

Using homology modeling, a computer model of a structural homolog or other polypeptide can be built or refined without crystallizing the molecule. For example, in another aspect, the present invention provides a computer-assisted method for homology modeling a structural homolog of a polypeptide of the invention including: aligning the amino acid sequence of a known or suspected structural homolog with the amino acid sequence of a polypeptide of the invention and incorporating the sequence of the homolog into a model of a polypeptide of the invention derived from atomic structure coordinates to yield a preliminary model of the homolog; subjecting the preliminary model to energy minimization to yield an energy minimized model; remodeling regions of the energy minimized model where stereochemistry restraints are violated to yield a final model of the homolog.

In another embodiment, the present invention contemplates a method for determining the crystal structure of a homolog of a polypeptide encoded by a subject amino acid sequence, or equivalent thereof, the method comprising: (a) providing the three dimensional structure of a crystallized polypeptide of a subject amino acid sequence, or a fragment thereof; (b) obtaining crystals of a homologous polypeptide comprising an amino acid sequence that is at least 80% identical to the subject amino acid sequence such that the three dimensional structure of the crystallized homologous polypeptide may be determined to a resolution of 3.5 Å or better; and (c) determining the three dimensional structure of the crystallized homologous polypeptide by x-ray crystallography based on the atomic coordinates of the three dimensional structure provided in step (a). In certain instances of the foregoing method, the atomic coordinates for the homologous polypeptide have a root mean square deviation from the backbone atoms of the polypeptide encoded by the applicable subject amino acid sequence, or a fragment thereof, of not more than 1.5 Å for all backbone atoms shared in common with the homologous polypeptide and the such encoded polypeptide, or a fragment thereof.

(vi) NMR Analysis Using X-Ray Structural Data

In another aspect, the structural coordinates of a known crystal structure may be applied to nuclear magnetic resonance data to determine the three dimensional structures of polypeptides with uncharacterized or incompletely characterized structure. (See for example, Wuthrich, 1986, John Wiley and Sons, New York: 176-199; Pflugrath et al., 1986, J. Molecular Biology 189: 383-386; Kline et al., 1986 J. Molecular Biology 189:377-382). While the secondary structure of a polypeptide may often be determined by NMR data, the spatial connections between individual pieces of secondary structure are not as readily determined. The structural coordinates of a polypeptide defined by x-ray crystallography can guide the NMR spectroscopist to an understanding of the spatial interactions between secondary structural elements in a polypeptide of related structure. Information on spatial interactions between secondary structural elements can greatly simplify NOE data from two-dimensional NMR experiments. In addition, applying the structural coordinates after the determination of secondary structure by NMR techniques simplifies the assignment of NOE's relating to particular amino acids in the polypeptide sequence.

In an embodiment, the invention relates to a method of determining three dimensional structures of polypeptides with unknown structures, by applying the structural coordinates of a crystal of the present invention to nuclear magnetic resonance data of the unknown structure. This method comprises the steps of: (a) determining the secondary structure of an unknown structure using NMR data; and (b) simplifying the assignment of through-space interactions of amino acids. The term “through-space interactions” defines the orientation of the secondary structural elements in the three dimensional structure and the distances between amino acids from different portions of the amino acid sequence. The term “assignment” defines a method of analyzing NMR data and identifying which amino acids give rise to signals in the NMR spectrum.

For all of this section on x-ray crystallography, see also Brooks et al. (1983) J Comput Chem 4:187-217; Weiner et al (1981) J. Comput. Chem. 106: 765; Eisenfield et al. (1991) Am J Physiol 261:C376-386; Lybrand (1991) J Pharm Belg 46:49-54; Froimowitz (1990). Biotechniques 8:640-644; Burbam et al. (1990) Proteins 7:99-111; Pedersen (1985) Environ Health Perspect 61:185-190; and Kini et al. (1991) J Biomol Struct Dyn 9:475-488; Ryckaert et al. (1977) J Comput Phys 23:327; Van Gunsteren et al. (1977) Mol Phys 34:1311; Anderson (1983) J Comput Phys 52:24; J. Mol. Biol. 48: 442-453, 1970; Dayhoff et al., Meth. Enzymol. 91: 524-545, 1983; Henikoff and Henikoff, Proc. Nat. Acad. Sci. USA 89: 10915-10919, 1992; J. Mol. Biol. 233: 716-738, 1993; Methods in Enzymology, Volume 276, Macromolecular crystallography, Part A, ISBN 0-12-182177-3 and Volume 277, Macromolecular crystallography, Part B, ISBN 0-12-182178-1, Eds. Charles W. Carter, Jr. and Robert M. Sweet (1997), Academic Press, San Diego; Pfuetzner, et al., J. Biol. Chem. 272: 430-434 (1997).

6. Interacting Proteins

The present invention also provides methods for isolating specific protein interactors of a polypeptide of the invention, and complexes comprising a polypeptide of the invention and one or more interacting proteins. In one aspect, the present invention contemplates an isolated protein complex comprising a polypeptide of the invention and at least one protein that interacts with the polypeptide of the invention. The interacting protein may be naturally-occurring. The interacting protein may be of the same origin of the polypeptide of the invention with which such protein interacts. Alternatively, the interacting protein may be of mammalian origin or human origin. Either the polypeptide of the invention, the interacting protein, or both, may be a fusion protein.

The present invention contemplates a method for identifying a protein capable of interacting with a polypeptide of the invention or a fragment thereof, the method comprising: (a) exposing a sample, to a solid substrate coupled to a polypeptide of the invention or a fragment thereof under conditions which promote protein-protein interactions; (b) washing the solid substrate so as to remove any polypeptides interacting non-specifically with the polypeptide or fragment; (c) eluting the polypeptides which specifically interact with the polypeptide or fragment; and (d) identifying the interacting protein. The sample may be an extract from the same bacterial species as the polypeptide of the invention of interest, a mammalian cell extract, a human cell extract, a purified protein (or a fragment thereof), or a mixture of purified proteins (or fragments thereof). The interacting protein may be identified by a number of methods, including mass spectrometry or protein sequencing.

In another aspect, the present invention contemplates a method for identifying a protein capable of interacting with a polypeptide of present invention or a fragment thereof, the method comprising: (a) subjecting a sample to protein-affinity chromatography on multiple columns, the columns having a polypeptide of the invention or a fragment thereof coupled to the column matrix in varying concentrations, and eluting bound components of the extract from the columns; (b) separating the components to isolate a polypeptide capable of interacting with the polypeptide or fragment; and (c) analyzing the interacting protein by mass spectrometry to identify the interacting protein. In certain instances, the foregoing method will use polyacrylamide gel electrophoresis without SDS.

In another aspect, the present invention contemplates a method for identifying a protein capable of interacting with a polypeptide of the invention, the method comprising: (a) subjecting a cellular extract or extracellular fluid to protein-affinity chromatography on multiple columns, the columns having a polypeptide of the invention or a fragment thereof coupled to the column matrix in varying concentrations, and eluting bound components of the extract from the columns; (b) gel-separating the components to isolate an interacting protein; wherein the interacting protein is observed to vary in amount in direct relation to the concentration of coupled polypeptide or fragment; (c) digesting the interacting protein to give corresponding peptides; (d) analyzing the peptides by MALDI-TOF mass spectrometry or post source decay to determine the peptide masses; and (d) performing correlative database searches with the peptide, or peptide fragment, masses, whereby the interacting protein is identified based on the masses of the peptides or peptide fragments. The foregoing method may include the further step of including the identifies of any interacting proteins into a relational database.

In another aspect, the invention further contemplates a method for identifying modulators of a protein complex, the method comprising: (a) contacting a protein complex comprising a polypeptide of the invention and an interacting protein with one or more test compounds; and (b) determining the effect of the test compound on (i) the activity of the protein complex, (ii) the amount of the protein complex, (iii) the stability of the protein complex, (iv) the conformation of the protein complex, (v) the activity of at least one polypeptide included in the protein complex, (vi) the conformation of at least one polypeptide included in the protein complex, (vii) the intracellular localization of the protein complex or a component thereof, (viii) the transcription level of a gene dependent on the complex, and/or (ix) the level of second messenger levels in a cell; thereby identifying modulators of the protein complex. The foregoing method may be carried out in vitro or in vivo as appropriate.

Typically, it will be desirable to immobilize a polypeptide of the invention to facilitate separation of complexes comprising a polypeptide of the invention from uncomplexed forms of the interacting proteins, as well as to accommodate automation of the assay. The polypeptide of the invention, or ligand, may be immobilized onto a solid support (e.g., column matrix, microtiter plate, slide, etc.). In certain embodiments, the ligand may be purified. In certain instances, a fusion protein may be provided which adds a domain that permits the ligand to be bound to a support.

In various in vitro embodiments, the set of proteins engaged in a protein-protein interaction comprises a cell extract, a clarified cell extract, or a reconstituted protein mixture of at least semi-purified proteins. By semi-purified, it is meant that the proteins utilized in the reconstituted mixture have been previously separated from other cellular or viral proteins. For instance, in contrast to cell lysates, the proteins involved in a protein-protein interaction are present in the mixture to at least about 50% purity relative to all other proteins in the mixture, and more preferably are present in greater, even 90-95%, purity. In certain embodiments of the subject method, the reconstituted protein mixture is derived by mixing highly purified proteins such that the reconstituted mixture substantially lacks other proteins (such as of cellular or viral origin) which might interfere with or otherwise alter the ability to measure activity resulting from the given protein-protein interaction.

Complex formation involving a polypeptide of the invention and another component polypeptide or a substrate polypeptide, may be detected by a variety of techniques. For instance, modulation in the formation of complexes can be quantitated using, for example, detectably labeled proteins (e.g. radiolabeled, fluorescently labeled, or enzymatically labeled), by immunoassay, or by chromatographic detection.

The present invention also provides assays for identifying molecules which are modulators of a protein-protein interaction involving a polypeptide of the invention, or are a modulator of the role of the complex comprising a polypeptide of the invention in the infectivity or pathogenicity of the pathogenic species of origin for such polypeptide. In one embodiment, the assay detects agents which inhibit formation or stabilization of a protein complex comprising a polypeptide of the invention and one or more additional proteins. In another embodiment, the assay detects agents which modulate the intrinsic biological activity of a protein complex comprising a polypeptide of the invention, such as an enzymatic activity, binding to other cellular components, cellular compartmentalization, signal transduction, and the like. Such modulators may be used, for example, in the treatment of diseases or disorders for the pathogenic species of origin for such polypeptide. In certain embodiments, the compound is a mechanism based inhibitor which chemically alters one member of a protein-protein interaction involving a polypeptide of the invention and which is a specific inhibitor of that member, e.g. has an inhibition constant about 10-fold, 100-fold, or 1000-fold different compared to homologous proteins.

In one embodiment, proteins that interact with a polypeptide of the invention may be isolated using immunoprecipitation. A polypeptide of the invention may be expressed in its pathogenic species of origin, or in a heterologous system. The cells expressing a polypeptide of the invention are then lysed under conditions which maintain protein-protein interactions, and complexes comprising a polypeptide of the invention are isolated. For example, a polypeptide of the invention may be expressed in mammalian cells, including human cells, in order to identify mammalian proteins that interact with a polypeptide of the invention and therefore may play a role in the infectivity or proliferation of such polypeptide's species of origin. In one embodiment, a polypeptide of the invention is expressed in the cell type for which it is desirable to find interacting proteins. For example, a polypeptide of the invention may be expressed in its species of origin in order to find interacting proteins derived from such species.

In an alternative embodiment, a polypeptide of the invention is expressed and purified and then mixed with a potential interacting protein or mixture of proteins to identify complex formation. The potential interacting protein may be a single purified or semi-purified protein, or a mixture of proteins, including a mixture of purified or semi-purified proteins, a cell lysate, a clarified cell lysate, a semi-purified cell lysate, etc.

In certain embodiments, it may be desirable to use a tagged version of a polypeptide of the invention in order to facilitate isolation of complexes from the reaction mixture. Suitable tags for immunoprecipitation experiments include HA, myc, FLAG, HIS, GST, protein A, protein G, etc. Immunoprecipitation from a cell lysate or other protein mixture may be carried out using an antibody specific for a polypeptide of the invention or using an antibody which recognizes a tag to which a polypeptide of the invention is fused (e.g., anti-HA, anti-myc, anti-FLAG, etc.). Antibodies specific for a variety of tags are known to the skilled artisan and are commercially available from a number of sources. In the case where a polypeptide of the invention is fused to a His, GST, or protein A/G tag, immunoprecipitation may be carried out using the appropriate affinity resin (e.g., beads functionalized with Ni, glutathione, Fc region of IgG, etc.). Test compounds which modulate a protein-protein interaction involving a polypeptide of the invention may be identified by carrying out the immunoprecipitation reaction in the presence and absence of the test agent and comparing the level and/or activity of the protein complex between the two reactions.

In another embodiment, proteins that interact with a polypeptide of the invention may be identified using affinity chromatography. Some examples of such chromatography are described in U.S. Ser. No. 09/727,812, filed Nov. 30, 2000, and the PCT Application filed Nov. 30, 2001 and entitled “Methods for Systematic Identification of Protein-Protein Interactions and other Properties”, which claims priority to such U.S. application.

In one aspect, for affinity chromatography using a solid support, a polypeptide of the invention or a fragment thereof may be attached by a variety of means known to those of skill in the art. For example, the polypeptide may be coupled directly (through a covalent linkage) to commercially available pre-activated resins as described in Formosa et al., Methods in Enzymology 1991, 208, 24-45; Sopta et al, J. Biol. Chem. 1985, 260, 10353-60; Archambault et al., Proc. Natl. Acad. Sci. USA 1997, 94, 14300-5. Alternatively, the polypeptide may be tethered to the solid support through high affinity binding interactions. If the polypeptide is expressed fused to a tag, such as GST, the fusion tag can be used to anchor the polypeptide to the matrix support, for example Sepharose beads containing immobilized glutathione. Solid supports that take advantage of these tags are commercially available.

In another aspect, the support to which a polypeptide may be immobilized is a soluble support, which may facilitate certain steps performed in the methods of the present invention. For example, the soluble support may be soluble in the conditions employed to create a binding interaction between a target and the polypeptide, and then used under conditions in which it is a solid for elution of the proteins or other biological materials that bind to a polypeptide.

The concentration of the coupled polypeptide may have an affect on the sensitivity of the method. In certain embodiments, to detect interactions most efficiently, the concentration of the polypeptide bound to the matrix should be at least 10-fold higher than the K_{d} of the interaction. Thus, the concentration of the polypeptide bound to the matrix should be highest for the detection of the weakest protein-protein interactions. However, if the concentration of the immobilized polypeptide is not as high as may be ideal, it may still be possible to observe protein-protein interactions of interest by, for example, increasing the concentration of the polypeptide or other moiety that interacts with the coupled polypeptide. The level of detection will of course vary with each different polypeptide, interactor, conditions of the assay, etc. In certain instances, the interacting protein binds to the polypeptide with a K_{d} of about 10⁻⁵ M to about 10⁻⁸ M or 10⁻¹⁰ M.

In another aspect, the coupling may be done at various ratios of the polypeptide to the resin. An upper limit of the protein:resin ratio may be determined by the isoelectric point and the ionic nature of the protein, although it may be possible to achieve higher polypeptide concentrations by use of various methods.

In certain embodiments, several concentrations of the polypeptide immobilized on a solid or soluble support may be used. One advantage of using multiple concentrations, although not a requirement, is that one may be able to obtain an estimate for the strength of the protein-protein interaction that is observed in the affinity chromatography experiment. Another advantage of using multiple concentrations is that a binding curve which has the proper shape may indicate that the interaction that is observed is biologically important rather than a spurious interaction with denatured protein.

In one example of such an embodiment, a series of columns may be prepared with varying concentrations of polypeptide (mg polypeptide/ml resin volume). The number of columns employed may be between 2 to 8, 10, 12, 15, 25 or more, each with a different concentration of attached polypeptide. Larger numbers of columns may be used if appropriate for the polypeptide being examined, and multiple columns may be used with the same concentration as any methods may require. In certain embodiments, 4 to 6 columns are prepared with varying concentrations of polypeptide. In another aspect of this embodiment, two control columns may be prepared: one that contains no polypeptide and a second that contains the highest concentration of polypeptide but is not treated with extract. After elution of the columns and separation of the eluent components (by one of the methods described below), it may be possible to distinguish the interacting proteins (if any) from the non-specific bound proteins as follows. The concentration of the interacting proteins, as determined by the intensity of the band on the gel, will increase proportionally to the increase in polypeptide concentration but will be missing from the second control column. This allows for the identification of unknown interacting proteins.

The method of the invention may be used for small-scale analysis. A variety of column sizes, types, and geometries may be used. In addition, other vessel shapes and sizes having a smaller scale than is usually found in laboratory experiments may be used as well, including a plurality of wells in a plate. For high throughput analysis, it is advantageous to use small volumes, from about 20, 30, 50, 80 or 100 μl. Larger or small volumes may be used, as necessary, and it may be possible to achieve high throughput analysis using them. The entire affinity chromatography procedure may be automated by assembling the micro-columns into an array (e.g. with 96 micro-column arrays).

A variety of materials may be used as the source of potential interacting proteins. In one embodiment, a cellular extract or extracellular fluid may be used. The choice of starting material for the extract may be based upon the cell or tissue type or type of fluid that would be expected to contain proteins that interact with the target protein. Micro-organisms or other organisms are grown in a medium that is appropriate for that organism and can be grown in specific conditions to promote the expression of proteins that may interact with the target protein. Exemplary starting material that may be used to make a suitable extract are: 1) one or more types of tissue derived from an animal, plant, or other multi-cellular organism, 2) cells grown in tissue culture that were derived from an animal or human, plant or other source, 3) micro-organisms grown in suspension or non-suspension cultures, 4) virus-infected cells; 5) purified organelles (including, but not restricted to nuclei, mitochondria, membranes, Golgi, endoplasmic reticulum, lysosomes, or peroxisomes) prepared by differential centrifugation or another procedure from animal, plant or other kinds of eukaryotic cells, 6) serum or other bodily fluids including, but not limited to, blood, urine, semen, synovial fluid, cerebrospinal fluid, amniotic fluid, lymphatic fluid or interstitial fluid. In other embodiments, a total cell extract may not be the optimal source of interacting proteins. For example, if the ligand is known to act in the nucleus, a nuclear extract can provide a 10-fold enrichment of proteins that are likely to interact with the ligand. In addition, proteins that are present in the extract in low concentrations may be enriched using another chromatographic method to fractionate the extract before screening various pools for an interacting protein.

Extracts are prepared by methods known to those of skill in the art. The extracts may be prepared at a low temperature (e.g., 4° C.) in order to retard denaturation or degradation of proteins in the extract. The pH of the extract may be adjusted to be appropriate for the body fluid or tissue, cellular, or organellar source that is used for the procedure (e.g. pH 7-8 for cytosolic extracts from mammals, but low pH for lysosomal extracts). The concentration of chaotropic or non-chaotropic salts in the extracting solution may be adjusted so as to extract the appropriate sets of proteins for the procedure. Glycerol may be added to the extract, as it aids in maintaining the stability of many proteins and also reduces background non-specific binding. Both the lysis buffer and column buffer may contain protease inhibitors to minimize proteolytic degradation of proteins in the extract and to protect the polypeptide. Appropriate co-factors that could potentially interact with the interacting proteins may be added to the extracting solution. One or more nucleases or another reagent may be added to the extract, if appropriate, to prevent protein-protein interactions that are mediated by nucleic acids. Appropriate detergents or other agents may be added to the solution, if desired, to extract membrane proteins from the cells or tissue. A reducing agent (e.g. dithiothreitol or 2-mercaptoethanol or glutathione or other agent) may be added. Trace metals or a chelating agent may be added, if desired, to the extracting solution.

Usually, the extract is centrifuged in a centrifuge or ultracentrifuge or filtered to provide a clarified supernatant solution. This supernatant solution may be dialyzed using dialysis tubing, or another kind of device that is standard in the art, against a solution that is similar to, but may not be identical with, the solution that was used to make the extract. The extract is clarified by centrifugation or filtration again immediately prior to its use in affinity chromatography.

In some cases, the crude lysate will contain small molecules that can interfere with the affinity chromatography. This can be remedied by precipitating proteins with ammonium sulfate, centrifugation of the precipitate, and re-suspending the proteins in the affinity column buffer followed by dialysis. An additional centrifugation of the sample may be needed to remove any particulate matter prior to application to the affinity columns.

The amount of cell extract applied to the column may be important for any embodiment. If too little extract is applied to the column and the interacting protein is present at low concentration, the level of interacting protein retained by the column may be difficult to detect. Conversely, if too much extract is applied to the column, protein may precipitate on the column or competition by abundant interacting proteins for the limited amount of protein ligand may result in a difficulty in detecting minor species.

The columns functionalized with a polypeptide of the invention are loaded with protein extract from an appropriate source that has been dialyzed against a buffer that is consistent with the nature of the expected interaction. The pH, salt concentrations and the presence or absence of reducing and chelating agents, trace metals, detergents, and co-factors may be adjusted according to the nature of the expected interaction. Most commonly, the pH and the ionic strength are chosen so as to be close to physiological for the source of the extract. The extract is most commonly loaded under gravity onto the columns at a flow rate of about 4-6 column volumes per hour, but this flow rate can be adjusted for particular circumstances in an automated procedure.

The volume of the extract that is loaded on the columns can be varied but is most commonly equivalent to about 5 to 10 column volumes. When large volumes of extract are loaded on the columns, there is often an improvement in the signal-to-noise ratio because more protein from the extract is available to bind to the protein ligand, whereas the background binding of proteins from the extract to the solid support saturates with low amounts of extract.

A control column may be included that contains the highest concentration of protein ligand, but buffer rather than extract is loaded onto this column. The elutions (eluates) from this column will contain polypeptide that failed to be attached to the column in a covalent manner, but no proteins that are derived from the extract.

The columns may be washed with a buffer appropriate to the nature of the interaction being analyzed, usually, but not necessarily, the same as the loading buffer. An elution buffer with an appropriate pH, glycerol, and the presence or absence of reducing agent, chelating agent, cofactors, and detergents are all important considerations. The columns may be washed with anywhere from about 5 to 20 column volumes of each wash buffer to eliminate unbound proteins from the natural extract. The flow rate of the wash is usually adjusted to about 4 to 6 column volumes per hour by using gravity or an automated procedure, but other flow rates are possible in specific circumstances.

In order to elute the proteins that have been retained by the column, the interactions between the extract proteins and the column ligand should be disrupted. This is performed by eluting the column with a solution of salt or detergent. Retention of activity by the eluted proteins may require the presence of glycerol and a buffer of appropriate p”, as well as proper choices of ionic strength and the presence or absence of appropriate reducing agent, chelating agent, trace metals, cofactors, detergents, chaotropic agents, and other reagents. If physical identification of the bound proteins is the objective, the elution may be performed sequentially, first with buffer of high ionic strength and then with buffer containing a protein denaturant, most commonly, but not restricted to sodium dodecyl sulfate (SDS), urea, or guanidine hydrochloride. In certain instances, the column is eluted with a protein denaturant, particularly SDS, for example as a 1% SDS solution. Using only the SDS wash, and omitting the salt wash, may result in SDS-gels that have higher resolution (sharper bands with less smearing). Also, using only the SDS wash results in half as many samples to analyze. The volume of the eluting solution may be varied but is normally about 2 to 4 column volumes. For 20 ml columns, the flow rate of the eluting procedures are most commonly about 4 to 6 column volumes per hour, under gravity, but can be varied in an automated procedure.

The proteins from the extract that were bound to and are eluted from the affinity columns may be most easily resolved for identification by an electrophoresis procedure, but this procedure may be modified, replaced by another suitable method, or omitted. Any of the denaturing or non-denaturing electrophoresis procedures that are standard in the art may be used for this purpose, including SDS-PAGE, gradient gels, capillary electrophoresis, and two-dimensional gels with isoelectric focusing in the first dimension and SDS-PAGE in the second. Typically, the individual components in the column eluent are separated by polyacrylamide gel electrophoresis.

After electrophoresis, protein bands or spots may be visualized using any number of methods know to those of skill in the art, including staining techniques such as Coomassie blue or silver staining, or some other agent that is standard in the art. Alternatively, autoradiography can be used for visualizing proteins isolated from organisms cultured on media containing a radioactive label, for example ³⁵SO₄²⁻ or ³⁵[S]methionine, that is incorporated into the proteins. The use of radioactively labeled extract allows a distinction to be made between extract proteins that were retained by the column and proteolytic fragments of the ligand that may be released from the column.

Protein bands that are derived from the extract (i.e. it did not elute from the control column that was not loaded with protein from the extract) and bound to an experimental column that contained polypeptide covalently attached to the solid support, and did not bind to a control column that did not contain any polypeptide, may be excised from the stained electrophoretic gel and further characterized.

To identify the protein interactor by mass spectrometry, it may be desirable to reduce the disulfide bonds of the protein followed by alkylation of the free thiols prior to digestion of the protein with protease. The reduction may be performed by treatment of the gel slice with a reducing agent, for example with dithiothreitol, whereupon, the protein is alkylated by treating the gel slice with a suitable alkylating agent, for example iodoacetamide.

Prior to analysis by mass spectrometry, the protein may be chemically or enzymatically digested. The protein sample in the gel slice may be subjected to in-gel digestion. Shevchenko A. et al., Mass Spectrometric Sequencing of Proteins from Silver Stained Polyacrylamide Gels. Analytical Chemistry 1996, 58, 850-858. One method of digestion is by treatment with the enzyme trypsin. The resulting peptides are extracted from the gel slice into a buffer.

The peptide fragments may be purified, for example by use of chromatography. A solid support that differentially binds the peptides and not the other compounds derived from the gel slice, the protease reaction or the peptide extract may be used. The peptides may be eluted from the solid support into a small volume of a solution that is compatible with mass spectrometry (e.g. 50% acetonitrile/0.1% trifluoroacetic acid).

The preparation of a protein sample from a gel slice that is suitable for mass spectrometry may also be done by an automated procedure.

Peptide samples derived from gel slices may be analyzed by any one of a variety of techniques in mass spectrometry as further described above. This technique may be used to assign function to an unknown protein based upon the known function of the interacting protein in the same or a homologous/orthologous organism.

Eluates from the affinity chromatography columns may also be analyzed directly without resolution by electrophoretic methods, by proteolytic digestion with a protease in solution, followed by applying the proteolytic digestion products to a reverse phase column and eluting the peptides from the column.

In yet another embodiment, proteins that interact with a polypeptide of the invention may be identified using an interaction trap assay (see also, U.S. Pat. No. 5,283,317; Zervos et al. (1993) Cell 72:223-232; Madura et al. (1993) J Biol Chem 268:12046-12054; Bartel et al. (1993) Biotechniques 14:920-924; and Iwabuchi et al. (1993) Oncogene 8:1693-1696).

In another embodiment, a method of the present invention makes use of chimeric genes which express hybrid proteins. To illustrate, a first hybrid gene comprises the coding sequence for a DNA-binding domain of a transcriptional activator fused in frame to the coding sequence for a “bait” protein, e.g., a polypeptide of the invention of sufficient length to bind to a potential interacting protein. The second hybrid protein encodes a transcriptional activation domain fused in frame to a gene encoding a “fish” protein, e.g., a potential interacting protein of sufficient length to interact with a polypeptide of the invention portion of the bait fusion protein. If the bait and fish proteins are able to interact, e.g., form a protein-protein interaction, they bring into close proximity the two domains of the transcriptional activator. This proximity causes transcription of a reporter gene which is operably linked to a transcriptional regulatory site responsive to the transcriptional activator, and expression of the reporter gene can be detected and used to score for the interaction of the bait and fish proteins.

In accordance with the present invention, the method includes providing a host cell, typically a yeast cell, e.g., Kluyverei lactis, Schizosaccharomyces pombe, Ustilago maydis, Saccharomyces cerevisiae, Neurospora crassa, Aspergillus niger, Aspergillus nidulans, Pichia pastoris, Candida tropicalis, and Hansenula polymorpha, though most preferably S cerevisiae or S. pombe. The host cell contains a reporter gene having a binding site for the DNA-binding domain of a transcriptional activator used in the bait protein, such that the reporter gene expresses a detectable gene product when the gene is transcriptionally activated. The first chimeric gene may be present in a chromosome of the host cell, or as part of an expression vector.

The host cell also contains a first chimeric gene which is capable of being expressed in the host cell. The gene encodes a chimeric protein, which comprises (a) a DNA-binding domain that recognizes the responsive element on the reporter gene in the host cell, and (b) a bait protein (e.g., a polypeptide of the invention).

A second chimeric gene is also provided which is capable of being expressed in the host cell, and encodes the “fish” fusion protein. In one embodiment, both the first and the second chimeric genes are introduced into the host cell in the form of plasmids. Preferably, however, the first chimeric gene is present in a chromosome of the host cell and the second chimeric gene is introduced into the host cell as part of a plasmid.

The DNA-binding domain of the first hybrid protein and the transcriptional activation domain of the second hybrid protein may be derived from transcriptional activators having separable DNA-binding and transcriptional activation domains. For instance, these separate DNA-binding and transcriptional activation domains are known to be found in the yeast GAL4 protein, and are known to be found in the yeast GCN4 and ADR1 proteins. Many other proteins involved in transcription also have separable binding and transcriptional activation domains which make them useful for the present invention, and include, for example, the LexA and VP16 proteins. It will be understood that other (substantially) transcriptionally-inert DNA-binding domains may be used in the subject constructs; such as domains of ACE1, λcI, lac repressor, jun or fos. In another embodiment, the DNA-binding domain and the transcriptional activation domain may be from different proteins. The use of a LexA DNA binding domain provides certain advantages. For example, in yeast, the LexA moiety contains no activation function and has no known affect on transcription of yeast genes. In addition, use of LexA allows control over the sensitivity of the assay to the level of interaction (see, for example, the Brent et al. PCT publication WO94/10300).

In certain embodiments, any enzymatic activity associated with the bait or fish proteins is inactivated, e.g., dominant negative or other mutants of a protein-protein interaction component can be used.

Continuing with the illustrative example, a polypeptide of the invention-mediated interaction, if any, between the bait and fish fusion proteins in the host cell, causes the activation domain to activate transcription of the reporter gene. The method is carried out by introducing the first chimeric gene and the second chimeric gene into the host cell, and subjecting that cell to conditions under which the bait and fish fusion proteins and are expressed in sufficient quantity for the reporter gene to be activated. The formation of a protein complex containing a polypeptide of the invention results in a detectable signal produced by the expression of the reporter gene.

In still further embodiments, the protein-protein interaction of interest is generated in whole cells, taking advantage of cell culture techniques to support the subject assay. For example, the protein-protein interaction of interest can be constituted in a prokaryotic or eukaryotic cell culture system. Advantages to generating the protein complex in an intact cell includes the ability to screen for inhibitors of the level or activity of the complex which are functional in an environment more closely approximating that which therapeutic use of the inhibitor would require, including the ability of the agent to gain entry into the cell. Furthermore, certain of the in vivo embodiments of the assay are amenable to high through-put analysis of candidate agents.

The components of the protein complex comprising a polypeptide of the invention can be endogenous to the cell selected to support the assay. Alternatively, some or all of the components can be derived from exogenous sources. For instance, fusion proteins can be introduced into the cell by recombinant techniques (such as through the use of an expression vector), as well as by microinjecting the fusion protein itself or mRNA encoding the fusion protein. Moreover, in the whole cell embodiments of the subject assay, the reporter gene construct can provide, upon expression, a selectable marker. Such embodiments of the subject assay are particularly amenable to high through-put analysis in that proliferation of the cell can provide a simple measure of the protein-protein interaction.

The amount of transcription from the reporter gene may be measured using any method known to those of skill in the art to be suitable. For example, specific mRNA expression may be detected using Northern blots or specific protein product may be identified by a characteristic stain, western blots or an intrinsic activity. In certain embodiments, the product of the reporter gene is detected by an intrinsic activity associated with that product. For instance, the reporter gene may encode a gene product that, by enzymatic activity, gives rise to a detection signal based on color, fluorescence, or luminescence.

The interaction trap assay of the invention may also be used to identify test agents capable of modulating formation of a complex comprising a polypeptide of the invention. In general, the amount of expression from the reporter gene in the presence of the test compound is compared to the amount of expression in the same cell in the absence of the test compound. Alternatively, the amount of expression from the reporter gene in the presence of the test compound may be compared with the amount of transcription in a substantially identical cell that lacks a component of the protein-protein interaction involving a polypeptide of the invention.

7. Antibodies

Another aspect of the invention pertains to antibodies specifically reactive with a polypeptide of the invention. For example, by using peptides based on a polypeptide of the invention, e.g., having a subject amino acid sequence or an immunogenic fragment thereof, antisera or monoclonal antibodies may be made using standard methods. An exemplary immunogenic fragment may contain eight, ten or more consecutive amino acid residues of a subject amino acid sequence. Certain fragments that are predicted to be immunogenic for the subject amino acid sequences (predicted) are set forth in the Tables contained in the Figures.

The term “antibody” as used herein is intended to include fragments thereof which are also specifically reactive with a polypeptide of the invention. Antibodies can be fragmented using conventional techniques and the fragments screened for utility in the same manner as is suitable for whole antibodies. For example, F(ab′)₂ fragments can be generated by treating antibody with pepsin. The resulting F(ab′)₂ fragment can be treated to reduce disulfide bridges to produce Fab′ fragments. The antibody of the present invention is further intended to include bispecific and chimeric molecules, as well as single chain (scFv) antibodies. Also within the scope of the invention are trimeric antibodies, humanized antibodies, human antibodies, and single chain antibodies. All of these modified forms of antibodies as well as fragments of antibodies are intended to be included in the term “antibody”.

In one aspect, the present invention contemplates a purified antibody that binds specifically to a polypeptide of the invention and which does not substantially cross-react with a protein which is less than about 80%, or less than about 90%, identical to a subject amino acid sequence. In another aspect, the present invention contemplates an array comprising a substrate having a plurality of address, wherein at least one of the addresses has disposed thereon a purified antibody that binds specifically to a polypeptide of the invention.

Antibodies may be elicited by methods known in the art. For example, a mammal such as a mouse, a hamster or rabbit may be immunized with an immunogenic form of a polypeptide of the invention (e.g., an antigenic fragment which is capable of eliciting an antibody response). Alternatively, immunization may occur by using a nucleic acid of the acid, which presumably in vivo expresses the polypeptide of the invention giving rise to the immunogenic response observed. Techniques for conferring immunogenicity on a protein or peptide include conjugation to carriers or other techniques well known in the art. For instance, a peptidyl portion of a polypeptide of the invention may be administered in the presence of adjuvant. The progress of immunization may be monitored by detection of antibody titers in plasma or serum. Standard ELISA or other immunoassays may be used with the immunogen as antigen to assess the levels of antibodies.

Following immunization, antisera reactive with a polypeptide of the invention may be obtained and, if desired, polyclonal antibodies isolated from the serum. To produce monoclonal antibodies, antibody producing cells (lymphocytes) may be harvested from an immunized animal and fused by standard somatic cell fusion procedures with immortalizing cells such as myeloma cells to yield hybridoma cells. Such techniques are well known in the art, and include, for example, the hybridoma technique (originally developed by Kohler and Milstein, (1975) Nature, 256: 495-497), as the human B cell hybridoma technique (Kozbar et al., (1983) Immunology Today, 4: 72), and the EBV-hybridoma technique to produce human monoclonal antibodies (Cole et al., (1985) Monoclonal Antibodies and Cancer Therapy, Alan R. Liss, Inc. pp. 77-96). Hybridoma cells can be screened immunochemically for production of antibodies specifically reactive with the polypeptides of the invention and the monoclonal antibodies isolated.

Antibodies directed against the polypeptides of the invention can be used to selectively block the action of the polypeptides of the invention. Antibodies against a polypeptide of the invention may be employed to treat infections, particularly bacterial infections and diseases. For example, the present invention contemplates a method for treating a subject suffering from a disease or disorder arising from a pathogenic species, comprising administering to an animal having the pathogen related condition a therapeutically effective amount of a purified antibody that binds specifically to a polypeptide of the invention from such pathogenic species. In another example, the present invention contemplates a method for inhibiting growth or infectivity of a pathogenic species, comprising contacting such species with a purified antibody that binds specifically to a polypeptide of the invention from such species.

In one embodiment, antibodies reactive with a polypeptide of the invention are used in the immunological screening of cDNA libraries constructed in expression vectors, such as λgt11, λgt18-23, λZAP, and λORF8. Messenger libraries of this type, having coding sequences inserted in the correct reading frame and orientation, can produce fusion proteins. For instance, λgt11 will produce fusion proteins whose amino termini consist of B-galactosidase amino acid sequences and whose carboxy termini consist of a foreign polypeptide. Antigenic epitopes of a polypeptide of the invention can then be detected with antibodies, as, for example, reacting nitrocellulose filters lifted from phage infected bacterial plates with an antibody specific for a polypeptide of the invention. Phage scored by this assay can then be isolated from the infected plate. Thus, homologs of a polypeptide of the invention can be detected and cloned from other sources.

Antibodies may be employed to isolate or to identify clones expressing the polypeptides to purify the polypeptides by affinity chromatography.

In other embodiments, the polypeptides of the invention may be modified so as to increase their immunogenicity. For example, a polypeptide, such as an antigenically or immunologically equivalent derivative, may be associated, for example by conjugation, with an immunogenic carrier protein for example bovine serum albumin (BSA) or keyhole limpet haemocyanin (KLH). Alternatively a multiple antigenic peptide comprising multiple copies of the protein or polypeptide, or an antigenically or immunologically equivalent polypeptide thereof may be sufficiently antigenic to improve immunogenicity so as to obviate the use of a carrier.

In other embodiments, the antibodies of the invention, or variants thereof, are modified to make them less immunogenic when administered to a subject. For example, if the subject is human, the antibody may be “humanized”; where the complimentarity determining region(s) of the hybridoma-derived antibody has been transplanted into a human monoclonal antibody, for example as described in Jones, P. et al. (1986), Nature 321, 522-525 or Tempest et al. (1991) Biotechnology 9, 266-273. Also, transgenic mice, or other mammals, may be used to express humanized antibodies. Such humanization may be partial or complete.

The use of a nucleic acid of the invention in genetic immunization may employ a suitable delivery method such as direct injection of plasmid DNA into muscles (Wolff et al., Hum Mol Genet 1992, 1:363, Manthorpe et al., Hum. Gene Ther. 1963:4, 419), delivery of DNA complexed with specific protein carriers (Wu et al., J Biol Chem. 1989: 264, 16985), coprecipitation of DNA with calcium phosphate (Benvenisty & Reshef, PNAS USA, 1986:83, 9551), encapsulation of DNA in various forms of liposomes (Kaneda et al., Science 1989:243, 375), particle bombardment (Tang et al., Nature 1992, 356:152, Eisenbraun et al., DNA Cell Biol 1993, 12:791) and in vivo infection using cloned retroviral vectors (Seeger et al., PNAS USA 1984:81, 5849).

8. Diagnostic Assays

The invention further provides a method for detecting the presence of a pathogenic species in a biological sample. Detection of a pathogenic species in a subject, particularly a mammal, and especially a human, will provide a diagnostic method for diagnosis of a disease or disorder related to such species. In general, the method involves contacting the biological sample with a compound or an agent capable of detecting a polypeptide of the invention or a nucleic acid of the invention. The term “biological sample” when used in reference to a diagnostic assay is intended to include tissues, cells and biological fluids isolated from a subject, as well as tissues, cells and fluids present within a subject.

The detection method of the invention may be used to detect the presence of a pathogenic species in a biological sample in vitro as well as in vivo. For example, in vitro techniques for detection of a nucleic acid of the invention include Northern hybridizations and in situ hybridizations. In vitro techniques for detection of polypeptides of the invention include enzyme linked immunosorbent assays (ELISAs), Western blots, immunoprecipitations, immunofluorescence, radioimmunoassays and competitive binding assays. Alternatively, polypeptides of the invention can be detected in vivo in a subject by introducing into the subject a labeled antibody specific for a polypeptide of the invention. For example, the antibody can be labeled with a radioactive marker whose presence and location in a subject can be detected by standard imaging techniques. It may be possible to use all of the diagnostic methods disclosed herein for pathogens in addition to the pathogenic speices of origin for any specific polypeptide of the invention.

Nucleic acids for diagnosis may be obtained from an infected individual's cells and tissues, such as bone, blood, muscle, cartilage, and skin. Nucleic acids, e.g., DNA and RNA, may be used directly for detection or may be amplified, e.g., enzymatically by using PCR or other amplification technique, prior to analysis. Using amplification, characterization of the species and strain of prokaryote present in an individual, may be made by an analysis of the genotype of the prokaryote gene. Deletions and insertions can be detected by a change in size of the amplified product in comparison to the genotype of a reference sequence. Point mutations can be identified by hybridizing a nucleic acid, e.g., amplified DNA, to a nucleic acid of the invention, which nucleic acid may be labeled. Perfectly matched sequences can be distinguished from mismatched duplexes by RNase digestion or by differences in melting temperatures. DNA sequence differences may also be detected by alterations in the electrophoretic mobility of the DNA fragments in gels, with or without denaturing agents, or by direct DNA sequencing. See, e.g. Myers et al., Science, 230: 1242 (1985). Sequence changes at specific locations also may be revealed by nuclease protection assays, such as RNase and S1 protection or a chemical cleavage method. See, e.g., Cotton et al., Proc. Natl. Acad. Sci., USA, 85: 4397-4401 (1985).

Agents for detecting a nucleic acid of the invention, e.g., comprising the sequence set forth in a subject nucleic acid sequence, include labeled or labelable nucleic acid probes capable of hybridizing to a nucleic acid of the invention. The nucleic acid probe can comprise, for example, the full length sequence of a nucleic acid of the invention, or an equivalent thereof, or a portion thereof, such as an oligonucleotide of at least 15, 30, 50, 100, 250 or 500 nucleotides in length and sufficient to specifically hybridize under stringent conditions to a subject nucleic acid sequence, or the complement thereof. Agents for detecting a polypeptide of the invention, e.g., comprising an amino acid sequence of a subject amino acid sequence, include labeled or labelable antibodies capable of binding to a polypeptide of the invention. Antibodies may be polyclonal, or alternatively, monoclonal. An intact antibody, or a fragment thereof (e.g., Fab or F(ab′)₂) can be used. Labeling the probe or antibody also encompasses direct labeling of the probe or antibody by coupling (e.g., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin.

In certain embodiments, detection of a nucleic acid of the invention in a biological sample involves the use of a probe/primer in a polymerase chain reaction (PCR) (see, e.g. U.S. Pat. Nos. 4,683,195 and 4,683,202), such as anchor PCR or RACE PCR, or, alternatively, in a ligation chain reaction (LCR) (see, e.g., Landegran et al. (1988) Science 241:1077-1080; and Nakazawa et al. (1994) PNAS 91:360-364), the latter of which can be particularly useful for distinguishing between orthologs of polynucleotides of the invention (see Abravaya et al. (1995) Nucleic Acids Res. 23:675-682). This method can include the steps of collecting a sample of cells from a patient, isolating nucleic acid (e.g., genomic, mRNA or both) from the cells of the sample, contacting the nucleic acid sample with one or more primers which specifically hybridize to a nucleic acid of the invention under conditions such that hybridization and amplification of the polynucleotide (if present) occurs, and detecting the presence or absence of an amplification product, or detecting the size of the amplification product and comparing the length to a control sample.

In one aspect, the present invention contemplates a method for detecting the presence of a pathogenic species in a sample, the method comprising: (a) providing a sample to be tested for the presence of such pathogenic species; (b) contacting the sample with an antibody reactive against eight consecutive amino acid residues of a subject amino acid sequence from such species under conditions which permit association between the antibody and its ligand; and (c) detecting interaction of the antibody with its ligand, thereby detecting the presence of such species in the sample.

In another aspect, the present invention contemplates a method for detecting the presence of a pathogenic species in a sample, the method comprising: (a) providing a sample to be tested for the presence of such pathogenic speices; (b) contacting the sample with an antibody that binds specifically to a polypeptide of the invention from such species under conditions which permit association between the antibody and its ligand; and (c) detecting interaction of the antibody with its ligand, thereby detecting the presence of such species in the sample.

In yet another example, the present invention contemplates a method for diagnosing a patient suffering from a disease or disorder of a pathogenic species, comprising: (a) obtaining a biological sample from a patient; (b) detecting the presence or absence of a polypeptide of the invention, or a nucleic acid encoding a polypeptide of the invention, in the sample; and (c) diagnosing a patient suffering from such a disease or disorder based on the presence of a polypeptide of the invention, or a nucleic acid encoding a polypeptide of the invention, in the patient sample.

The diagnostic assays of the invention may also be used to monitor the effectiveness of a anti-pathogenic treatment in an individual suffering from a disease or disorder of such pathogen. For example, the presence and/or amount of a nucleic acid of the invention or a polypeptide of the invention can be detected in an individual suffering from a disease or disorder related to a pathogen before and after treatment with an anti-pathogen therapeutic agent. Any change in the level of a polynucleotide or polypeptide of the invention after treatment of the individual with the therapeutic agent can provide information about the effectiveness of the treatment course. In particular, no change, or a decrease, in the level of a polynucleotide or polypeptide of the invention present in the biological sample will indicate that the therapeutic is successfully combating such disease or disorder.

The invention also encompasses kits for detecting the presence of a pathogen in a biological sample. For example, the kit can comprise a labeled or labelable compound or agent capable of detecting a polynucleotide or polypeptide of the invention in a biological sample; means for determining the amount of a pathogen in the sample; and means for comparing the amount of a pathogen in the sample with a standard. The compound or agent can be packaged in a suitable container. The kit can further comprise instructions for using the kit to detect a polynucleotide or polypeptide of the invention.

9. Drug Discovery

Modulators to polypeptides of the invention and other structurally related molecules, and complexes containing the same, may be identified and developed as set forth below and otherwise using techniques and methods known to those of skill in the art. The modulators of the invention may be employed, for instance, to inhibit and treat diseases or conditions associated with the pathogne of origin for any such polypeptide of the invention.

A variety of methods for inhibiting the growth or infectivity of pathogens are contemplated by the present invention. For example, exemplary methods involve contacting a pathogen with a polypeptide of the invention which modulates the same or another polypeptide from such pathogen, a nucleic acid encoding such polypeptide of the invention, or a compound thought or shown to be effective against such pathogen.

For example, in one aspect, the present invention contemplates a method for treating a patient suffering from an infection of a pathognic species, comprising administering to the patient an inhibitor of a subject amino acid sequence from such species in an amount effective to inhibit the expression and/or activity of a polypeptide of the invention. In certain instances, the animal is a human or a livestock animal such as a cow, pig, goat or sheep. The present invention further contemplates a method for treating a subject suffering from a disease or disorder of a pathogen, comprising administering to an animal having the condition a therapeutically effective amount of a molecule identified using one of the methods of the present invention.

The present invention contemplates making any molecule that is shown to modulate the activity of a polypeptide of the invention.

In another embodiment, inhibitors, modulators of the subject polypeptides, or biological complexes containing them, may be used in the manufacture of a medicament for any number of uses, including, for example, treating any disease or other treatable condition of a patient (including humans and animals).

(a) Drug Design

A number of techniques can be used to screen, identify, select and design chemical entities capable of associating with polypeptides of the invention, structurally homologous molecules, and other molecules. Knowledge of the structure for a polypeptide of the invention, determined in accordance with the methods described herein, permits the design and/or identification of molecules and/or other modulators which have a shape complementary to the conformation of a polypeptide of the invention, or more particularly, a druggable region thereof. It is understood that such techniques and methods may use, in addition to the exact structural coordinates and other information for a polypeptide of the invention, structural equivalents thereof described above (including, for example, those structural coordinates that are derived from the structural coordinates of amino acids contained in a druggable region as described above).

The term “chemical entity,” as used herein, refers to chemical compounds, complexes of two or more chemical compounds, and fragments of such compounds or complexes. In certain instances, it is desirable to use chemical entities exhibiting a wide range of structural and functional diversity, such as compounds exhibiting different shapes (e.g., flat aromatic rings(s), puckered aliphatic rings(s), straight and branched chain aliphatics with single, double, or triple bonds) and diverse functional groups (e.g., carboxylic acids, esters, ethers, amines, aldehydes, ketones, and various heterocyclic rings).

In one aspect, the method of drug design generally includes computationally evaluating the potential of a selected chemical entity to associate with any of the molecules or complexes of the present invention (or portions thereof). For example, this method may include the steps of (a) employing computational means to perform a fitting operation between the selected chemical entity and a druggable region of the molecule or complex; and (b) analyzing the results of said fitting operation to quantify the association between the chemical entity and the druggable region.

A chemical entity may be examined either through visual inspection or through the use of computer modeling using a docking program such as GRAM, DOCK, or AUTODOCK (Dunbrack et al., Folding & Design, 2:27-42 (1997)). This procedure can include computer fitting of chemical entities to a target to ascertain how well the shape and the chemical structure of each chemical entity will complement or interfere with the structure of the subject polypeptide (Bugg et al., Scientific American, December: 92-98 (1993); West et al., TIPS, 16:67-74 (1995)). Computer programs may also be employed to estimate the attraction, repulsion, and steric hindrance of the chemical entity to a druggable region, for example. Generally, the tighter the fit (e.g., the lower the steric hindrance, and/or the greater the attractive force) the more potent the chemical entity will be because these properties are consistent with a tighter binding constant. Furthermore, the more specificity in the design of a chemical entity the more likely that the chemical entity will not interfere with related proteins, which may minimize potential side-effects due to unwanted interactions.

A variety of computational methods for molecular design, in which the steric and electronic properties of druggable regions are used to guide the design of chemical entities, are known: Cohen et al. (1990) J. Med. Cam. 33: 883-894; Kuntz et al. (1982) J. Mol. Biol. 161: 269-288; DesJarlais (1988) J. Med. Cam. 31: 722-729; Bartlett et al. (1989) Spec. Publ., Roy. Soc. Chem. 78: 182-196; Goodford et al. (1985) J. Med. Cam. 28: 849-857; and DesJarlais et al. J. Med. Cam. 29: 2149-2153. Directed methods generally fall into two categories: (1) design by analogy in which 3-D structures of known chemical entities (such as from a crystallographic database) are docked to the druggable region and scored for goodness-of-fit; and (2) de novo design, in which the chemical entity is constructed piece-wise in the druggable region. The chemical entity may be screened as part of a library or a database of molecules. Databases which may be used include ACD (Molecular Designs Limited), NCI (National Cancer Institute), CCDC (Cambridge Crystallographic Data Center), CAST (Chemical Abstract Service), Derwent (Derwent Information Limited), Maybridge (Maybridge Chemical Company Ltd), Aldrich (Aldrich Chemical Company), DOCK (University of California in San Francisco), and the Directory of Natural Products (Chapman & Hall). Computer programs such as CONCORD (Tripos Associates) or DB-Converter (Molecular Simulations Limited) can be used to convert a data set represented in two dimensions to one represented in three dimensions.

Chemical entities may be tested for their capacity to fit spatially with a druggable region or other portion of a target protein. As used herein, the term “fits spatially” means that the three-dimensional structure of the chemical entity is accommodated geometrically by a druggable region. A favorable geometric fit occurs when the surface area of the chemical entity is in close proximity with the surface area of the druggable region without forming unfavorable interactions. A favorable complementary interaction occurs where the chemical entity interacts by hydrophobic, aromatic, ionic, dipolar, or hydrogen donating and accepting forces. Unfavorable interactions may be steric hindrance between atoms in the chemical entity and atoms in the druggable region.

If a model of the present invention is a computer model, the chemical entities may be positioned in a druggable region through computational docking. If, on the other hand, the model of the present invention is a structural model, the chemical entities may be positioned in the druggable region by, for example, manual docking. As used herein the term “docking” refers to a process of placing a chemical entity in close proximity with a druggable region, or a process of finding low energy conformations of a chemical entity/druggable region complex.

In an illustrative embodiment, the design of potential modulator begins from the general perspective of shape complimentary for the druggable region of a polypeptide of the invention, and a search algorithm is employed which is capable of scanning a database of small molecules of known three-dimensional structure for chemical entities which fit geometrically with the target druggable region. Most algorithms of this type provide a method for finding a wide assortment of chemical entities that are complementary to the shape of a druggable region of the subject polypeptide. Each of a set of chemical entities from a particular data-base, such as the Cambridge Crystallographic Data Bank (CCDB) (Allen et al. (1973) J. Chem. Doc. 13: 119), is individually docked to the druggable region of a polypeptide of the invention in a number of geometrically permissible orientations with use of a docking algorithm. In certain embodiments, a set of computer algorithms called DOCK, can be used to characterize the shape of invaginations and grooves that form the active sites and recognition surfaces of the druggable region (Kuntz et al. (1982) J. Mol. Biol 161: 269-288). The program can also search a database of small molecules for templates whose shapes are complementary to particular binding sites of a polypeptide of the invention (DesJarlais et al. (1988) J Med Chem 31: 722-729).

The orientations are evaluated for goodness-of-fit and the best are kept for further examination using molecular mechanics programs, such as AMBER or CHARMM. Such algorithms have previously proven successful in finding a variety of chemical entities that are complementary in shape to a druggable region.

Goodford (1985, J Med Chem 28:849-857) and Boobbyer et al. (1989, J Med Chem 32:1083-1094) have produced a computer program (GRID) which seeks to determine regions of high affinity for different chemical groups (termed probes) of the druggable region. GRID hence provides a tool for suggesting modifications to known chemical entities that might enhance binding. It may be anticipated that some of the sites discerned by GRID as regions of high affinity correspond to “pharmacophoric patterns” determined inferentially from a series of known ligands. As used herein, a “pharmacophoric pattern” is a geometric arrangement of features of chemical entities that is believed to be important for binding. Attempts have been made to use pharmacophoric patterns as a search screen for novel ligands (Jakes et al. (1987) J Mol Graph 5:41-48; Brint et al. (1987) J Mol Graph 5:49-56; Jakes et al. (1986) J Mol Graph 4:12-20).

Yet a further embodiment of the present invention utilizes a computer algorithm such as CLIX which searches such databases as CCDB for chemical entities which can be oriented with the druggable region in a way that is both sterically acceptable and has a high likelihood of achieving favorable chemical interactions between the chemical entity and the surrounding amino acid residues. The method is based on characterizing the region in terms of an ensemble of favorable binding positions for different chemical groups and then searching for orientations of the chemical entities that cause maximum spatial coincidence of individual candidate chemical groups with members of the ensemble. The algorithmic details of CLIX is described in Lawrence et al. (1992) Proteins 12:3141.

In this way, the efficiency with which a chemical entity may bind to or interfere with a druggable region may be tested and optimized by computational evaluation. For example, for a favorable association with a druggable region, a chemical entity must preferably demonstrate a relatively small difference in energy between its bound and fine states (i.e., a small deformation energy of binding). Thus, certain, more desirable chemical entities will be designed with a deformation energy of binding of not greater than about 10 kcal/mole, and more preferably, not greater than 7 kcal/mole. Chemical entities may interact with a druggable region in more than one conformation that is similar in overall binding energy. In those cases, the deformation energy of binding is taken to be the difference between the energy of the free entity and the average energy of the conformations observed when the chemical entity binds to the target.

In this way, the present invention provides computer-assisted methods for identifying or designing a potential modulator of the activity of a polypeptide of the invention including: supplying a computer modeling application with a set of structure coordinates of a molecule or complex, the molecule or complex including at least a portion of a druggable region from a polypeptide of the invention; supplying the computer modeling application with a set of structure coordinates of a chemical entity; and determining whether the chemical entity is expected to bind to the molecule or complex, wherein binding to the molecule or complex is indicative of potential modulation of the activity of a polypeptide of the invention.

In another aspect, the present invention provides a computer-assisted method for identifying or designing a potential modulator to a polypeptide of the invention, supplying a computer modeling application with a set of structure coordinates of a molecule or complex, the molecule or complex including at least a portion of a druggable region of a polypeptide of the invention; supplying the computer modeling application with a set of structure coordinates for a chemical entity, evaluating the potential binding interactions between the chemical entity and active site of the molecule or molecular complex; structurally modifying the chemical entity to yield a set of structure coordinates for a modified chemical entity, and determining whether the modified chemical entity is expected to bind to the molecule or complex, wherein binding to the molecule or complex is indicative of potential modulation of the polypeptide of the invention.

In one embodiment, a potential modulator can be obtained by screening a peptide library (Scott and Smith, Science, 249:386-390 (1990); Cwirla et al., Proc. Natl. Acad. Sci., 87:6378-6382 (1990); Devlin et al., Science, 249:404-406 (1990)). A potential modulator selected in this manner could then be systematically modified by computer modeling programs until one or more promising potential drugs are identified. Such analysis has been shown to be effective in the development of HIV protease inhibitors (Lam et al., Science 263:380-384 (1994); Wlodawer et al., Ann. Rev. Biochem. 62:543-585 (1993); Appelt, Perspectives in Drug Discovery and Design 1:23-48 (1993); Erickson, Perspectives in Drug Discovery and Design 1:109-128 (1993)). Alternatively a potential modulator may be selected from a library of chemicals such as those that can be licensed from third parties, such as chemical and pharmaceutical companies. A third alternative is to synthesize the potential modulator de novo.

For example, in certain embodiments, the present invention provides a method for making a potential modulator for a polypeptide of the invention, the method including synthesizing a chemical entity or a molecule containing the chemical entity to yield a potential modulator of a polypeptide of the invention, the chemical entity having been identified during a computer-assisted process including supplying a computer modeling application with a set of structure coordinates of a molecule or complex, the molecule or complex including at least one druggable region from a polypeptide of the invention; supplying the computer modeling application with a set of structure coordinates of a chemical entity; and determining whether the chemical entity is expected to bind to the molecule or complex at the active site, wherein binding to the molecule or complex is indicative of potential modulation. This method may further include the steps of evaluating the potential binding interactions between the chemical entity and the active site of the molecule or molecular complex and structurally modifying the chemical entity to yield a set of structure coordinates for a modified chemical entity, which steps may be repeated one or more times.

Once a potential modulator is identified, it can then be tested in any standard assay for the macromolecule depending of course on the macromolecule, including in high throughput assays. Further refinements to the structure of the modulator will generally be necessary and can be made by the successive iterations of any and/or all of the steps provided by the particular screening assay, in particular further structural analysis by e.g., ¹⁵N NMR relaxation rate determinations or x-ray crystallography with the modulator bound to the subject polypeptide. These studies may be performed in conjunction with biochemical assays.

Once identified, a potential modulator may be used as a model structure, and analogs to the compound can be obtained. The analogs are then screened for their ability to bind the subject polypeptide. An analog of the potential modulator might be chosen as a modulator when it binds to the subject polypeptide with a higher binding affinity than the predecessor modulator.

In a related approach, iterative drug design is used to identify modulators of a target protein. Iterative drug design is a method for optimizing associations between a protein and a modulator by determining and evaluating the three dimensional structures of successive sets of protein/modulator complexes. In iterative drug design, crystals of a series of protein/modulator complexes are obtained and then the three-dimensional structures of each complex is solved. Such an approach provides insight into the association between the proteins and modulators of each complex. For example, this approach may be accomplished by selecting modulators with inhibitory activity, obtaining crystals of this new protein/modulator complex, solving the three dimensional structure of the complex, and comparing the associations between the new protein/modulator complex and previously solved protein/modulator complexes. By observing how changes in the modulator affected the protein/modulator associations, these associations may be optimized.

In addition to designing and/or identifying a chemical entity to associate with a druggable region, as described above, the same techniques and methods may be used to design and/or identify chemical entities that either associate, or do not associate, with affinity regions, selectivity regions or undesired regions of protein targets. By such methods, selectivity for one or a few targets, or alternatively for multiple targets, from the same species or from multiple species, can be achieved.

For example, a chemical entity may be designed and/or identified for which the binding energy for one druggable region, e.g., an affinity region or selectivity region, is more favorable than that for another region, e.g., an undesired region, by about 20%, 30%, 50% to about 60% or more. It may be the case that the difference is observed between (a) more than two regions, (b) between different regions (selectivity, affinity or undesirable) from the same target, (c) between regions of different targets, (d) between regions of homologs from different species, or (e) between other combinations. Alternatively, the comparison may be made by reference to the K_{d}, usually the apparent K_{d}, of said chemical entity with the two or more regions in question.

In another aspect, prospective modulators are screened for binding to two nearby druggable regions on a target protein. For example, a modulator that binds a first region of a target polypeptide does not bind a second nearby region. Binding to the second region can be determined by monitoring changes in a different set of amide chemical shifts in either the original screen or a second screen conducted in the presence of a modulator (or potential modulator) for the first region. From an analysis of the chemical shift changes, the approximate location of a potential modulator for the second region is identified. Optimization of the second modulator for binding to the region is then carried out by screening structurally related compounds (e.g., analogs as described above). When modulators for the first region and the second region are identified, their location and orientation in the ternary complex can be determined experimentally. On the basis of this structural information, a linked compound, e.g., a consolidated modulator, is synthesized in which the modulator for the first region and the modulator for the second region are linked. In certain embodiments, the two modulators are covalently linked to form a consolidated modulator. This consolidated modulator may be tested to determine if it has a higher binding affinity for the target than either of the two individual modulators. A consolidated modulator is selected as a modulator when it has a higher binding affinity for the target than either of the two modulators. Larger consolidated modulators can be constructed in an analogous manner, e.g., linking three modulators which bind to three nearby regions on the target to form a multilinked consolidated modulator that has an even higher affinity for the target than the linked modulator. In this example, it is assumed that is desirable to have the modulator bind to all the druggable regions. However, it may be the case that binding to certain of the druggable regions is not desirable, so that the same techniques may be used to identify modulators and consolidated modulators that show increased specificity based on binding to at least one but not all druggable regions of a target.

The present invention provides a number of methods that use drug design as described above. For example, in one aspect, the present invention contemplates a method for designing a candidate compound for screening for inhibitors of a polypeptide of the invention, the method comprising: (a) determining the three dimensional structure of a crystallized polypeptide of the invention or a fragment thereof; and (b) designing a candidate inhibitor based on the three dimensional structure of the crystallized polypeptide or fragment.

In another aspect, the present invention contemplates a method for identifying a potential inhibitor of a polypeptide of the invention, the method comprising: (a) providing the three-dimensional coordinates of a polypeptide of the invention or a fragment thereof; (b) identifying a druggable region of the polypeptide or fragment; and (c) selecting from a database at least one compound that comprises three dimensional coordinates which indicate that the compound may bind the druggable region; (d) wherein the selected compound is a potential inhibitor of a polypeptide of the invention.

In another aspect, the present invention contemplates a method for identifying a potential modulator of a molecule comprising a druggable region similar to that of a subject amino acid sequence, the method comprising: (a) using the atomic coordinates of amino acid residues from a subject amino acid sequence, or a fragment thereof, ± a root mean square deviation from the backbone atoms of the amino acids of not more than 1.5 Å, to generate a three-dimensional structure of a molecule comprising a subject amino acid sequence-like druggable region; (b) employing the three dimensional structure to design or select the potential modulator; (c) synthesizing the modulator; and (d) contacting the modulator with the molecule to determine the ability of the modulator to interact with the molecule.

In another aspect, the present invention contemplates an apparatus for determining whether a compound is a potential inhibitor of a polypeptide having a subject amino acid sequence, the apparatus comprising: (a) a memory that comprises: (i) the three dimensional coordinates and identities of the atoms of a polypeptide of the invention or a fragment thereof that form a druggable site; and (ii) executable instructions; and (b) a processor that is capable of executing instructions to: (i) receive three-dimensional structural information for a candidate compound; (ii) determine if the three-dimensional structure of the candidate compound is complementary to the structure of the interior of the druggable site; and (iii) output the results of the determination.

In another aspect, the present invention contemplates a method for designing a potential compound for the prevention or treatment of a pathogenic disease or disorder, the method comprising: (a) providing the three dimensional structure of a crystallized polypeptide of the invention, or a fragment thereof; (b) synthesizing a potential compound for the prevention or treatment of such disease or disorder based on the three dimensional structure of the crystallized polypeptide or fragment; (c) contacting a polypeptide of the invention or such pathogenic species with the potential compound; and (d) assaying the activity of a polypeptide of the invention, wherein a change in the activity of the polypeptide indicates that the compound may be useful for prevention or treatment of such disease or disorder.

In another aspect, the present invention contemplates a method for designing a potential compound for the prevention or treatment of a pathogenic disease or disorder, the method comprising: (a) providing structural information of a druggable region derived from NMR spectroscopy of a polypeptide of the invention, or a fragment thereof; (b) synthesizing a potential compound for the prevention or treatment of such disease or disorder based on the structural information; (c) contacting a polypeptide of the invention or such species with the potential compound; and (d) assaying the activity of a polypeptide of the invention, wherein a change in the activity of the polypeptide indicates that the compound may be useful for prevention or treatment of such disease or disorder.

(b) In Vitro Assays

Polypeptides of the invention may be used to assess the activity of small molecules and other modulators in in vitro assays. In one embodiment of such an assay, agents are identified which modulate the biological activity of a protein, protein-protein interaction of interest or protein complex, such as an enzymatic activity, binding to other cellular components, cellular compartmentalization, signal transduction, and the like. In certain embodiments, the test agent is a small organic molecule.

Assays may employ kinetic or thermodynamic methodology using a wide variety of techniques including, but not limited to, microcalorimetry, circular dichroism, capillary zone electrophoresis, nuclear magnetic resonance spectroscopy, fluorescence spectroscopy, and combinations thereof.

The invention also provides a method of screening compounds to identify those which modulate the action of polypeptides of the invention, or polynucleotides encoding the same. The method of screening may involve high-throughput techniques. For example, to screen for modulators, a synthetic reaction mix, a cellular compartment, such as a membrane, cell envelope or cell wall, or a preparation of any thereof, comprising a polypeptide of the invention and a labeled substrate or ligand of such polypeptide is incubated in the absence or the presence of a candidate molecule that may be a modulator of a polypeptide of the invention. The ability of the candidate molecule to modulate a polypeptide of the invention is reflected in decreased binding of the labeled ligand or decreased production of product from such substrate. Detection of the rate or level of production of product from substrate may be enhanced by using a reporter system. Reporter systems that may be useful in this regard include but are not limited to colorimetric labeled substrate converted into product, a reporter gene that is responsive to changes in a nucleic acid of the invention or polypeptide activity, and binding assays known in the art.

Another example of an assay for a modulator of a polypeptide of the invention is a competitive assay that combines a polypeptide of the invention and a potential modulator with molecules that bind to a polypeptide of the invention, recombinant molecules that bind to a polypeptide of the invention, natural substrates or ligands, or substrate or ligand mimetics, under appropriate conditions for a competitive inhibition assay. Polypeptides of the invention can be labeled, such as by radioactivity or a colorimetric compound, such that the number of molecules of a polypeptide of the invention bound to a binding molecule or converted to product can be determined accurately to assess the effectiveness of the potential modulator.

A number of methods for identifying a molecule which modulates the activity of a polypeptide are known in the art. For example, in one such method, a subject polypeptide is contacted with: a test compound, and the activity of the subject polypeptide in the presence of the test compound is determined, wherein a change in the activity of the subject polypeptide is indicative that the test compound modulates the activity of the subject polypeptide. In certain instances, the test compound agonizes the activity of the subject polypeptide, and in other instances, the test compound antagonizes the activity of the subject polypeptide.

In another example, a compound which modulates the growth or infectivity of a pathogen may be identified by (a) contacting a polypeptide of the invention from such pathogen with a test compound; and (b) determining the activity of the polypeptide in the presence of the test compound, wherein a change in the activity of the polypeptide is indicative that the test compound may modulate the growth or infectivity of such pathogen.

(c) In Vivo Assays

Animal models of bacterial infection and/or disease may be used as an in vivo assay for evaluating the effectiveness of a potential drug target in treating or preventing diseases or disorders. A number of suitable animal models are described briefly below, however, these models are only examples and modifications, or completely different animal models, may be used in accord with the methods of the invention.

(i) Mouse Soft Tissue Model

The mouse soft tissue infection model is a sensitive and effective method for measurement of bacterial proliferation. In these models (Vogelman et al., 1988, J. Infect. Dis. 157: 287-298) anesthetized mice are infected with the bacteria in the muscle of the hind thigh. The mice can be either chemically immune compromised (e.g., cytoxan treated at 125 mg/kg on days −4, −2, and 0) or immunocompetent. The dose of microbe necessary to cause an infection is variable and depends on the individual microbe, but commonly is on the order of 10⁵-10⁶ colony forming units per injection for bacteria. A variety of mouse strains are useful in this model although Swiss Webster and DBA2 lines are most commonly used. Once infected the animals are conscious and show no overt ill effects of the infections for approximately 12 hours. After that time virulent strains cause swelling of the thigh muscle, and the animals can become bacteremic within approximately 24 hours. This model most effectively measures proliferation of the microbe, and this proliferation is measured by sacrifice of the infected animal and counting colonies from homogenized thighs.

(ii) Diffusion Chamber Model

A second model useful for assessing the virulence of microbes is the diffusion chamber model (Malouin et al., 1990, Infect. Immun. 58: 1247-1253; Doy et al., 1980, J. Infect. Dis. 2: 39-51; Kelly et al., 1989, Infect. Immun. 57: 344-350. In this model rodents have a diffusion chamber surgically placed in the peritoneal cavity. The chamber consists of a polypropylene cylinder with semipermeable membranes covering the chamber ends. Diffusion of peritoneal fluid into and out of the chamber provides nutrients for the microbes. The progression of the “infection” may be followed by examining growth, the exoproduct production or RNA messages. The time experiments are done by sampling multiple chambers.

(iii) Endocarditis Model

For bacteria, an important animal model effective in assessing pathogenicity and virulence is the endocarditis model (J. Santoro and M. E. Levinson, 1978, Infect. Immun. 19: 915-918). A rat endocarditis model can be used to assess colonization, virulence and proliferation.

(iv) Osteomyelitis Model

A fourth model useful in the evaluation of pathogenesis is the osteomyelitis model (Spagnolo et al., 1993, Infect. Immun. 61: 5225-5230). Rabbits are used for these experiments. Anesthetized animals have a small segment of the tibia removed and microorganisms are microinjected into the wound. The excised bone segment is replaced and the progression of the disease is monitored. Clinical signs, particularly inflammation and swelling are monitored. Termination of the experiment allows histolic and pathologic examination of the infection site to complement the assessment procedure.

(v) Murine Septic Arthritis Model

A fifth model relevant to the study of microbial pathogenesis is a murine septic arthritis model (Abdelnour et al., 1993, Infect. Immun. 61: 3879-3885). In this model mice are infected intravenously and pathogenic organisms are found to cause inflammation in distal limb joints. Monitoring of the inflammation and comparison of inflammation vs. inocula allows assessment of the virulence of related strains.

(vi) Bacterial Peritonitis Model

Finally, bacterial peritonitis offers rapid and predictive data on the virulence of strains (M. G. Bergeron, 1978, Scand. J. Infect. Dis. Suppl. 14: 189-206; S. D. Davis, 1975, Antimicrob. Agents Chemother. 8: 50-53). Peritonitis in rodents, such as mice, can provide essential data on the importance of targets. The end point may be lethality or clinical signs can be monitored. Variation in infection dose in comparison to outcome allows evaluation of the virulence of individual strains.

A variety of other in vivo models are available and may be used when appropriate for specific pathogens or specific test agents. For example, target organ recovery assays (Gordee et al., 1984, J. Antibiotics 37:1054-1065; Bannatyne et al., 1992, Infect. 20:168-170) may be useful for fungi and for bacterial pathogens which are not acutely virulent to animals.

It is also relevant to note that the species of animal used for an infection model, and the specific genetic make-up of that animal, may contribute to the effective evaluation of the effects of a particular test agent. For example, immuno-incompetent animals may, in some instances, be preferable to immuno-competent animals. For example, the action of a competent immune system may, to some degree, mask the effects of the test agent as compared to a similar infection in an immuno-incompetent animal. In addition, many opportunistic infections, in fact, occur in immuno-compromised patients, so modeling an infection in a similar immunological environment is appropriate.

10. Vaccines

There are provided by the invention, products, compositions and methods for raising immunological response against a pathogen, especially those pathogens of origin for the polypeptides of the invention. In one aspect, a polypeptide of the invention or a nucleic acid of the invention, or an antigenic fragment thereof, may be administered to a subject, optionally with a booster, adjuvant, or other composition that stimulates immune responses.

Another aspect of the invention relates to a method for inducing an immunological response in an individual, particularly a mammal which comprises inoculating the individual with a polypeptide of the invention and/or a nucleic acid of the invention, adequate to produce antibody and/or T cell immune response to protect said individual from infection, particularly bacterial infection. Also provided are methods whereby such immunological response slows bacterial replication. Yet another aspect of the invention relates to a method of inducing immunological response in an individual which comprises delivering to such individual a nucleic acid vector, sequence or ribozyme to direct expression of a polypeptide of the invention and/or a nucleic acid of the invention in vivo in order to induce an immunological response, such as, to produce antibody and/or T cell immune response, including, for example, cytokine-producing T cells or cytotoxic T cells, to protect said individual, preferably a human, from disease, whether that disease is already established within the individual or not. One example of administering the gene is by accelerating it into the desired cells as a coating on particles or otherwise. Such nucleic acid vector may comprise DNA, RNA, a ribozyme, a modified nucleic acid, a DNA/RNA hybrid, a DNA-protein complex or an RNA-protein complex.

A further aspect of the invention relates to an immunological composition that when introduced into an individual, preferably a human, capable of having induced within it an immunological response, induces an immunological response in such individual to a nucleic acid of the invention and/or a polypeptide encoded therefrom, wherein the composition comprises a recombinant nucleic acid of the invention and/or polypeptide encoded therefrom and/or comprises DNA and/or RNA which encodes and expresses an antigen of said nucleic acid of the invention, polypeptide encoded therefrom, or other polypeptide of the invention. The immunological response may be used therapeutically or prophylactically and may take the form of antibody immunity and/or cellular immunity, such as cellular immunity arising from CTL or CD4+T cells.

In another embodiment, the invention relates to compositions comprising a polypeptide of the invention and an adjuvant. The adjuvant can be any vehicle which would typically enhance the antigenicity of a polypeptide, e.g., minerals (for instance, alum, aluminum hydroxide or aluminum phosphate), saponins complexed to membrane protein antigens (immune stimulating complexes), pluronic polymers with mineral oil, killed mycobacteria in mineral oil, Freund's complete adjuvant, bacterial products, such as muramyl dipeptide (MDP) and lipopolysaccharide (LPS), as well as lipid A, liposomes, or any of the other adjuvants known in the art. A polypeptide of the invention can be emulsified with, absorbed onto, or coupled with the adjuvant.

A polypeptide of the invention may be fused with co-protein or chemical moiety which may or may not by itself produce antibodies, but which is capable of stabilizing the first protein and producing a fused or modified protein which will have antigenic and/or immunogenic properties, and preferably protective properties. Thus fused recombinant protein, may further comprise an antigenic co-protein, such as lipoprotein D from Hemophilus influenzae, Glutathione-S-transferase (GST) or beta-galactosidase, or any other relatively large co-protein which solubilizes the protein and facilitates production and purification thereof. Moreover, the co-protein may act as an adjuvant in the sense of providing a generalized stimulation of the immune system of the organism receiving the protein. The co-protein may be attached to either the amino- or carboxy-terminus of a polypeptide of the invention.

Provided by this invention are compositions, particularly vaccine compositions, and methods comprising the polypeptides and/or polynucleotides of the invention and immunostimulatory DNA sequences, such as those described in Sato, Y. et al. Science 273: 352 (1996).

Also, provided by this invention are methods using the described polynucleotide or particular fragments thereof, which have been shown to encode non-variable regions of bacterial cell surface proteins, in polynucleotide constructs used in such genetic immunization experiments in animal models of infection with a pathogen of interest. Such experiments will be particularly useful for identifying protein epitopes able to provoke a prophylactic or therapeutic immune response. It is believed that this approach will allow for the subsequent preparation of monoclonal antibodies of particular value, derived from the requisite organ of the animal successfully resisting or clearing infection, for the development of prophylactic agents or therapeutic treatments of bacterial infection in mammals, particularly humans.

A polypeptide of the invention may be used as an antigen for vaccination of a host to produce specific antibodies which protect against invasion of bacteria, for example by blocking adherence of bacteria to damaged tissue.

11. Array Analysis

In part, the present invention is directed to the use of subject nucleic acids in arrays to assess gene expression. In another part, the present invention is directed to the use of subject nucleic acids in arrays for their pathogen of origin. In yet another part, the present invention contemplates using the subject nucleic acids to interact with probes contained on arrays.

In one aspect, the present invention contemplates an array comprising a substrate having a plurality of addresses, wherein at least one of the addresses has disposed thereon a capture probe that can specifically bind to a nucleic acid of the invention. In another aspect, the present invention contemplates a method for detecting expression of a nucleotide sequence which encodes a polypeptide of the invention, or a fragment thereof, using the foregoing array by: (a) providing a sample comprising at least one mRNA molecule; (b) exposing the sample to the array under conditions which promote hybridization between the capture probe disposed on the array and a nucleic acid complementary thereto; and (c) detecting hybridization between an mRNA molecule of the sample and the capture probe disposed on the array, thereby detecting expression of a sequence which encodes for a polypeptide of the invention, or a fragment thereof.

Arrays are often divided into microarrays and macroarrays, where microarrays have a much higher density of individual probe species per area Microarrays may have as many as 1000 or more different probes in a 1 cm² area. There is no concrete cut-off to demarcate the difference between micro- and macroarrays, and both types of arrays are contemplated for use with the invention.

Microarrays are known in the art and generally consist of a surface to which probes that correspond in sequence to gene products (e.g., cDNAs, mRNAs, oligonucleotides) are bound at known positions. In one embodiment, the microarray is an array (e.g., a matrix) in which each position represents a discrete binding site for a product encoded by a gene (e.g., a protein or RNA), and in which binding sites are present for products of most or almost all of the genes in the organism's genome. In certain embodiments, the binding site or site is a nucleic acid or nucleic acid analogue to which a particular cognate cDNA can specifically hybridize. The nucleic acid or analogue of the binding site may be, e.g., a synthetic oligomer, a full-length cDNA, a less-than full length cDNA, or a gene fragment.

Although in certain embodiments the microarray contains binding sites for products of all or almost all genes in the target organism's genome, such comprehensiveness is not necessarily required. Usually the microarray will have binding sites corresponding to at least 100, 500, 1000, 4000 genes or more. In certain embodiments, arrays will have anywhere from about 50, 60, 70, 80, 90, or even more than 95% of the genes of a particular organism represented. The microarray typically has binding sites for genes relevant to testing and confirming a biological network model of interest. Several exemplary human microarrays are publicly available.

The probes to be affixed to the arrays are typically polynucleotides. These DNAs can be obtained by, e.g., polymerase chain reaction (PCR) amplification of gene segments from genomic DNA, cDNA (e.g., by RT-PCR), or cloned sequences. PCR primers are chosen, based on the known sequence of the genes or cDNA, that result in amplification of unique fragments (e.g., fragments that do not share more than 10 bases of contiguous identical sequence with any other fragment on the microarray). Computer programs are useful in the design of primers with the required specificity and optimal amplification properties. See, e.g., Oligo p1 version 5.0 (National Biosciences). In an alternative embodiment, the binding (hybridization) sites are made from plasmid or phage clones of genes, cDNAs (e.g., expressed sequence tags), or inserts therefrom (Nguyen et al., 1995, Genomics 29:207-209).

A number of methods are known in the art for affixing the nucleic acids or analogues to a solid support that makes up the array (Schena et al., 1995, Science 270:467-470; DeRisi et al., 1996, Nature Genetics 14:457-460; Shalon et al., 1996, Genome Res. 6:639-645; and Schena et al., 1995, Proc. Natl. Acad. Sci. USA 93:10539-11286).

Another method for making microarrays is by making high-density oligonucleotide arrays (Fodor et al., 1991, Science 251:767-773; Pease et al., 1994, Proc. Natl. Acad. Sci. USA 91:5022-5026; Lockhart et al., 1996, Nature Biotech 14:1675; U.S. Pat. Nos. 5,578,832; 5,556,752; and 5,510,270; Blanchard et al., 1996, 11: 687-90).

Other methods for making microarrays, e.g., by masking (Maskos and Southern, 1992, Nuc. Acids Res. 20:1679-1684), may also be used. In principal, any type of array, for example, dot blots on a nylon hybridization membrane (see Sambrook et al., Molecular Cloning—A Laboratory Manual (2nd Ed.), Vol. 1-3, Cold Spring Harbor Laboratory, Cold Spring Harbor, N.Y., 1989), could be used, although, as will be recognized by those of skill in the art.

The nucleic acids to be contacted with the microarray may be prepared in a variety of ways, and may include nucleotides of the subject invention. Such nucleic acids are often labeled fluorescently. Nucleic acid hybridization and wash conditions are chosen so that the population of labeled nucleic acids will specifically hybridize to appropriate, complementary nucleic acids affixed to the matrix. Non-specific binding of the labeled nucleic acids to the array can be decreased by treating the array with a large quantity of non-specific DNA—a so-called “blocking” step.

When fluorescently labeled probes are used, the fluorescence emissions at each site of a transcript array may be detected by scanning confocal laser microscopy. When two fluorophores are used, a separate scan, using the appropriate excitation line, is carried out for each of the two fluorophores used. Fluorescent microarray scanners are commercially available from Affymetrix, Packard BioChip Technologies, BioRobotics and many other suppliers. Signals are recorded, quantitated and analyzed using a variety of computer software.

According to the method of the invention, the relative abundance of an mRNA in two cells or cell lines is scored as a perturbation and its magnitude determined (i.e., the abundance is different in the two sources of mRNA tested), or as not perturbed (i.e., the relative abundance is the same). As used herein, a difference between the two sources of RNA of at least a factor of about 25% (RNA from one source is 25% more abundant in one source than the other source), more usually about 50%, even more often by a factor of about 2 (twice as abundant), 3 (three times as abundant) or 5 (five times as abundant) is scored as a perturbation. Present detection methods allow reliable detection of difference of an order of about 2-fold to about 5-fold, but more sensitive methods are expected to be developed.

In addition to identifying a perturbation as positive or negative, it is advantageous to determine the magnitude of the perturbation. This can be carried out, as noted above, by calculating the ratio of the emission of the two fluorophores used for differential labeling, or by analogous methods that will be readily apparent to those of skill in the art.

In certain embodiments, the data obtained from such experiments reflects the relative expression of each gene represented in the microarray. Expression levels in different samples and conditions may now be compared using a variety of statistical methods.

12. Pharmaceutical Compositions

Pharmaceutical compositions of this invention include any modulator identified according to the present invention, or a pharmaceutically acceptable salt thereof, and a pharmaceutically acceptable carrier, adjuvant, or vehicle. The term “pharmaceutically acceptable carrier” refers to a carrier(s) that is “acceptable” in the sense of being compatible with the other ingredients of a composition and not deleterious to the recipient thereof.

Methods of making and using such pharmaceutical compositions are also included in the invention. The pharmaceutical compositions of the invention can be administered orally, parenterally, by inhalation spray, topically, rectally, nasally, buccally, vaginally, or via an implanted reservoir. The term parenteral as used herein includes subcutaneous, intracutaneous, intravenous, intramuscular, intra articular, intrasynovial, intrasternal, intrathecal, intralesional, and intracranial injection or infusion techniques.

Dosage levels of between about 0.01 and about 100 mg/kg body weight per day, preferably between about 0.5 and about 75 mg/kg body weight per day of the modulators described herein are useful for the prevention and treatment of disease and conditions, including diseases and conditions mediated by pathogenic speices of origin for the polypeptides of the invention. The amount of active ingredient that may be combined with the carrier materials to produce a single dosage form will vary depending upon the host treated and the particular mode of administration. A typical preparation will contain from about 5% to about 95% active compound (w/w). Alternatively, such preparations contain from about 20% to about 80% active compound.

13. Antimicrobial Agents

The polypeptides of the invention may be used to develop antimicrobial agents for use in a wide variety of applications. The uses are as varied as surface disinfectants, topical pharmaceuticals, personal hygiene applications (e.g., antimicrobial soap, deodorant or the like), additives to cell culture medium, and systemic pharmaceutical products. Antimicrobial agents of the invention may be incorporated into a wide variety of products and used to treat an already existing microbial infection/contamination or may be used prophylactically to suppress future infection/contamination.

The antimicrobial agents of the invention may be administered to a site, or potential site, of infection/contamination in either a liquid or solid form. Alternatively, the agent may be applied as a coating to a surface of an object where microbial growth is undesirable using nonspecific absorption or covalent attachment. For example, implants or devices (such as linens, cloth, plastics, heart pacemakers, surgical stents, catheters, gastric tubes, endotracheal tubes, prosthetic devices) can be coated with the antimicrobials to minimize adherence or persistence of bacteria during storage and use. The antimicrobials may also be incorporated into such devices to provide slow release of the agent locally for several weeks during healing. The antimicrobial agents may also be used in association with devices such as ventilators, water reservoirs, air-conditioning units, filters, paints, or other substances. Antimicrobials of the invention may also be given orally or systemically after transplantation, bone replacement, during dental procedures, or during implantation to prevent colonization with bacteria.

In another embodiment, antimicrobial agents of the invention may be used as a food preservative or in treating food products to eliminate potential pathogens. The latter use might be targeted to the fish and poultry industries that have serious problems with enteric pathogens which cause severe human disease. In a further embodiment, the agents of the invention may be used as antimicrobials for food crops, either as agents to reduce post harvest spoilage or to enhance host resistance. The antimicrobials may also be used as preservatives in processed foods either alone or in combination with antibacterial food additives such as lysozymes.

In another embodiment, the antimicrobials of the invention may be used as an additive to culture medium to prevent or eliminate infection of cultured cells with a pathogen.

### EXEMPLIFICATION

The invention now being generally described, it will be more readily understood by reference to the following examples which are included merely for purposes of illustration of certain aspects and embodiments of the present invention, and are not intended to limit the invention in any way.

### Example 1

### Isolation and Cloning of Nucleic Acid

Staphylococcus aureus is a Gram-positive cocci that is implicated in a wide number of skin infections, and is of particular concern in hospitals and other health institutions. The high virulence of the organism and the ability of many strains to resist numerous anti-microbial agents, presents difficult therapeutic issues. S. aureus polynucleotide sequences were obtained from The Institute of Genomic Research (TIGR) (Rockville, Md.; www.tigr.org). S. aureus genomic DNA is extracted from a crushed cell pellet (strain ColA) and subjected to 10% sucrose and 2% SDS in a 60° C. water bath, followed by the addition of 1 M NaCl for a 40 minute incubation on ice. Impurities, including RNA and proteins, are removed by enzymatic degradation via RNAse and phenol-chloroform extractions, respectively. The DNA is then precipitated, washed with ethanol, and quantified by UV absorption.

Helicobacter pylori is a Gram-negative spiral bacteria infecting approximately 50% of the world's population. It is the only known microorganism to inhabit the human stomach. It causes chronic gastritis and duodenal and gastric ulcers. As well, it has been implicated in gastric cancer and non-Hodgkin's lymphoma. Recently it has been characterized as a group I carcinogen by the World Health Organization. H. pylori polynucleotide sequences were obtained from NCBI at ftp://ncbi.nlm.nih.gov/genbank/genomes/Bacteria/Helicobacter_{—}pylori_{—}26695/. H. pylori chromosomal DNA was acquired from the American Type Culture Collection (ATCC; reference # 43504D).

Escherichia coli is a rod shaped Gram-negative bacteria found ubiquitously in the human intestinal tract. When this bacteria spreads to sites outside the intestinal tract, it can cause disease. It is responsible for three types of infections in humans: urinary tract infections (UTI), neonatal meningitis, and intestinal diseases (gastroenteritis). E. coli Polynucleotide sequences were obtained from NCBI at ftp://ncbi.nlm.nih.gov/genbank/genomes/Bacteria/Escherichia_{—}coli_K12/. E. coli DNA is extracted from a crushed cell pellet (strain K12) and subjected to 10% sucrose and 2% SDS in a 60° C. water bath, followed by the addition of 1 M NaCl for a 40 minute incubation on ice. The impurities, including RNA and proteins were removed by enzymatic degradation via RNAse, and phenol-chloroform extractions, respectively. The DNA was precipitated, washed with ethanol, and quantified by UV absorption.

Streptococcus pneumoniae are paired, alpha-hemolytic, Gram-positive cocci. It is the leading cause of bacterial pneumonia and it is also implicated as a significant pathogenic agent in the development of bronchial infections, sinusitis and meningitis. The increasing prevalence of strains that are resistant to anti-microbial agents makes this an even more deadly pathogen. Polynucleotide sequences were obtained from The Institute of Genomic Research (TIGR) (Rockville, Md.; www.tigr.org). DNA is extracted from a crushed cell pellet and and subjected to 10% sucrose and 2% SDS in a 60° C. water bath, followed by the addition of 1 M NaCl for a 40 minute incubation on ice. The impurities, including RNA and proteins, were removed by enzymatic degradation via RNAse, and phenol-chloroform extractions, respectively. The DNA was precipitated, washed with ethanol, and quantified by UV absorption.

Pseudomonas aeruginosa is an opportunistic Gram-negative bacilli found in sewage, plants, and sometimes the intestine. It is capable of infecting various organs and has been identified in numerous infections including those in the ears, lungs, urinary tract, blood and in burns and surgical wound infections. Polynucleotide sequences were obtained from The Institute of Genomic Research (TIGR) (Rockville, Md.; www.tigr.org). Chromosomal DNA was acquired from the American Type Culture Collection (ATCC; reference #17933D).

Enterococcus faecalis is a facultative Gram-positive anaerobe bacteria that is associated with both community and hospital acquired infections. Approximately 80% of enteroccocal infections in humans are caused by E. faecalis. The most common enterococcal-associated nosocomial infections are infections of the urinary tract, followed by surgical wound infections and bacteremia. Other enterococcal infections include intra abdominal and pelvic infections, central nervous system infections, and in rare instances, osteomyelitis and pulmonary infections. The high virulence of the organism and the ability of many strains to resist numerous anti-microbial agents, presents difficult therapeutic issues. Most enterococci are relatively resistant to penicillin, ampicillin, and the ureidopenicillins. E. faecalis polynucleotide sequences were obtained from The Institute of Genomic Research (TIGR) (Rockville, Md.; www.tigr.org). E. faecalis genomic DNA is extracted from a crushed cell pellet (strain V583) and and subjected to 10% sucrose and 2% SDS in a 60° C. water bath, followed by the addition of 1 M NaCl for a 40 minute incubation on ice. Impurities, including RNA and proteins, are removed by enzymatic degradation via RNAse and phenol-chloroform extractions, respectively. The DNA is then precipitated, washed with ethanol, and quantified by UV absorption.

The coding sequences of the subject nucleic acid sequences (predicted) are obtained by reference to either publicly available databases or from the use of a bioinformatics program that is used to select the coding sequence of interest from the applicable genome. For example, bioinformatics programs that may be used to select the coding sequence of interest from the genome of S. aureus include that described in Nucleic Acids Research, 1999, 27:4636-4641 and the ContigExpress and Translate functionalities of Vector NTI Suite (InforMax). For example, coding sequences for the genome of H. pylori may be obtained from NCBI (http://www.ncbi.nlm.nih.gov/cgi-bin/Entrez/altik?gi=128&db=Genome). For example, coding sequences for the genome of E. coli may be obtained from NCBI (http://www.ncbi.nlm.nih.gov/cgi-bin/Entrez/altik?gi=115&db=Genome). For example, bioinformatics programs that may be used to select the coding sequence of interest from the genome of S. pneumoniae include that described in Nucleic Acids Research, 1999, 27:4636-4641 and the ContigExpress and Translate functionalities of Vector NTI Suite (InforMax). For example, coding sequences for the genome of P. aeruginosa may be obtained from NCBI (http://www.ncbi.nlm.nih.gov/cgi-bin/Entrez/framik?db=Genome&gi=163). For example, bioinformatics programs that may be used to select the coding sequence of interest from the genome of E. faecalis include that described in Nucleic Acids Research, 1999, 27:4636-4641 and the ContigExpress and Translate functionalities of Vector NTI Suite (InforMax).

The subject nucleic acid sequences (experimental) are amplified from purified genomic DNA using PCR with primers that are identified with a computer program using the corresponding subject nucleic acid sequences (predicted). The PCR primers are selected so as to introduce restriction enzyme cleavage sites at the flanking regions of the DNA (e.g., Ndel and BglII). The nucleic acid sequences for the forward and reverse primers for each of the subject nucleic acid sequences (experimental) are shown in the appropriate Figures, as described above, with their respective restriction sites and melting temperatures shown in the applicable Table contained in the Figures.

The PCR reaction for each of the subject nucleic acid sequences (experimental) is performed using 50-100 ng of chromosomal DNA and 2 Units of a high fidelity DNA Polymerase (for example Pfu Turbo (Stratagene) or Platinum Pfx (Invitrogen)). The thermocycling conditions for the PCR process include a DNA melting step at 94° C. for 45 sec, a primer annealing step at 48° C.-58° C. (depending on Primer [Tm]) for 45 sec, and an extension step at 68° C.-72° C. (depending on enzyme) for 1 min 45 sec-2 min 30 sec (depending on size of DNA). After 25-30 cycles, a final blocking step at 72° C. for 9 min is carried out. The amplified nucleic acid product is isolated from the PCR cocktail using silica-gel membrane based column chromatography (Qiagen). The quality of the PCR product is assessed by resolving an aliquot of amplified product on a 1% agarose gel. The DNA is quantified spectrophotometrically at A₂₆₀ or by visualizing the resolved genes with a 302 nm UV-B light source.

The PCR product for each of the subject nucleic acid sequences (experimental) is directionally cloned into the polylinker region of any of three expression vectors: pET28 (Novagen), pET15 (Novagen) or pGEX (Pharmacia/LKB Biotechnology). Additional restriction enzyme sites may be engineered into the expression vectors to allow for simultaneous clones to be prepared having different purification tags. After the ligation reaction, the DNA is transformed into competent E. coli cells (Strains XL1-Blue (Stratagene) or DH5α (Invitrogen)) via heat shock or electroporation as described in Sambrook, et al., Molecular Cloning: A Laboratory Manual, 2^{nd} Ed., Cold Spring Harbor Laboratory Press, Cold Spring Harbor, N.Y. (1989). The expression vectors contain the bacteriophage T7 promoter for RNA polymerase, and the E. coli strain used produces T7 RNA polymerase upon induction with isopropyl-β-D-thiogalactoside (IPTG). The sequence of the cloning site adds a Glutathione S-transferase (GST) tag, or a polyhistidine (6×His) tag, at the N- or C-terminus of the recombinant protein. The cloning site also inserts a cleavage site for the thrombin or Tev (Invitrogen) enzymes between the recombinant protein and the N- or C-terminal GST or polyhistidine tag.

Transformants are selected using the appropriate antibiotic (Ampicillin or Kanamycin) and identified using PCR, or another method, to analyze their DNA. The polynucleotide sequence cloned into the expression construct is then isolated using a modified alkaline lysis method (Birnboim, H. C., and Doly, J. (1979) Nucl. Acids Res. 7, 1513-1522.) The sequence of the clone is verified by standard polynucleotide sequencing methods. The various nucleic and amino acid sequences for the different polypeptides of the invention are presented in the Figures.

The expression construct containing a subject nucleic acid (experimental) is transformed into a bacterial host strain BL21-Gold (DE3) supplemented with a plasmid called pUBS520, which directs expression of tRNA for arginine (agg and aga) and serves to augment the expression of the recombinant protein in the host cell (Gene, vol. 85 (1989) 109-114). The expression construct may also be transformed into BL21-Gold (DE3) without pUBS520, BL21-Gold (DE3) Codon-Plus (RIL) or (RP) (Stratagene) or Roseatta (DE3) (Novagen), the latter two of which contain genes encoding tRNAs. Alternatively, the expression construct may be transformed into BL21 STAR E. coli (Invitrogen) cells which has an Rnase deficiency that reduces degradation of recombinant mRNA transcript and therefore increases the protein yield. The recombinant protein is then assayed for positive overexpression in the host and the presence of the protein in the cytoplasmic (water soluble) region of the cell.

### Example 2

### Test Protein Expression and Solubility

(a) Test Expression

Transformed cells are grown in LB medium supplemented with the appropriate antibiotics up to a final concentration of 100 μg/ml. The cultures are shaken at 37° C. until they reach an optical density (OD₆₀₀) between 0.6 and 0.7. The cultures are then induced with isopropyl-beta-D-thiogalactopyranoside (IPTG) to a final concentration of 0.5 mM at 15° C. for 10 hours, 25° C. for 4 hours, or 30° C. for 4 hours.

(b) Method One for Determining Protein Solubility Levels

The cells are harvested by centrifugation and subjected to a freeze/thaw cycle. The cells are lysed using detergent, sonication, or incubation with lysozyme. Total and soluble proteins are assayed using a 26-well BioRad Criterion gel running system. The proteins are stained with an appropriate dye (Coomassie, Silver stain, or Sypro-Red) and visualized with the appropriate visualization system. Typically, recombinant protein is seen as a prominent band in the lanes of the gel representing the soluble fraction.

(c) Method Two for Determining Protein Solubility Levels

The soluble and insoluble fractions (in the presence of 6M urea) of the cell pellet are bound to the appropriate affinity column. The purified proteins from both fractions are analysed by SDS-PAGE and the levels of protein in the soluble fraction are determined The approximate percent solubility of a polypeptide of a subject amino acid sequence (experimental) is determined using one of the two foregoing methods, and the resulting percent solubility is presented in the applicable Table contained in the Figures.

### Example 3

### Native Protein Expression

The expression construct clone comprising one of the subject amino acid sequences (experimental) is introduced into an expression host. The resultant cell line is then grown in culture. The method of growth is dependant on whether the protein to be purified is a native protein or a labeled protein. For native and ¹⁵N labeled protein production, a Gold-pUBS520 (as described above), BL21-Gold (DE3) Codon-Plus (RIL) or (RP), or BL21 STAR E. Coli cell line is used. For generating proteins metabolically labeled with selenium, the clone is introduced into a strain called B834 (Novagen). The methods for expressing labeled polypeptides of the invention are described in the Examples that follow.

In one method for expressing an unlabeled polypepetide of the invention, 2 L LB cultures or 1 L TB cultures are inoculated with a 1% (v/v) starter culture (OD₆₀₀ of 0.8). The cultures are shaken at 37° C. and 200 rpm and grown to an OD₆₀₀ of 0.6-0.8 followed by induction with 0.5 mM IPTG at 15° C. and 200 rpm for at least 10 hours or at 25° C. for 4 hours. The cells are harvested by centrifugation and the pellets are resuspended in 25 ml HEPES buffer (50 mM, pH 7.5), supplemented with 100 μl of protease inhibitors (PMSF and benzamidine (Sigma)) and flash-frozen in liquid nitrogen.

Alternatively, for an unlabeled polypeptide of the invention, a starter culture is prepared in a 300 mL Tunair flask (Shelton Scientific) by adding 20 mL of medium having 47.6 g/L of Terrific Broth and 1.5% glycerol in dH₂O followed by autoclaving for 30 minutes at 121° C. and 15 psi. When the broth cools to room temperature, the medium is supplemented with 6.3 μM CoCl₂-6H₂O, 33.2 μM MnSO₄-5H₂O, 5.9 μM CuCl₂-2H₂O, 8.1 μM H₃BO₃, 8.3 μM Na₂MoO₄-2H₂O, 7 μM ZnSO₄-7H₂O, 108 μM FeSO₄-7H₂O, 68 μM CaCl₂-2H₂O, 4.1 μM AlCl₃-6H₂O, 8.4 μM NiCl₂-6H₂O, 1 mM MgSO₄, 0.5% v/v of Kao and Michayluk vitamins mix (Sigma; Cat. No. K3129), 25 μg/mL Carbenicillin, and 50 μg/mL Kanamycin. The medium is then inoculated with several colonies of the freshly transformed expression construct of interest. The culture is incubated at 37° C. and 260 rpm for about 3 hours and then transferred to a 2.5 L Tunair Flask containing 1 L of the above media. The 1 L culture is then incubated at 37° C. with shaking at 230-250 rpm on an orbital shaker having a 1 inch orbital diameter. When the culture reaches an OD₆% of 3-6 it is induced with 0.5 mM IPTG. The induced culture is then incubated at 15° C. with shaking at 230-250 rpm or faster for about 6-15 hours. The cells are harvested by centrifugation at 3500 rpm at 4° C. for 20 minutes and the cell pellet is resuspended in 15 mL ice cold binding buffer (Hepes 50 mM, pH 7.5) and 100 μl of protease inhibitors (50 mM PMSF and 100 mM Benzamidine, stock concentration) and flash frozen.

### Example 4

### Expression of Selmet Labeled Polypeptides

The cell harboring a plasmid with the nucleic acid sequence of interest is inoculated into 20 ml of NMM (New Minimal Medium) and shaken at 37° C. for 8-9 hours. This culture is then transferred into a 6 L Erlenmeyer flask containing 2 L of minimum medium (M9). The media is supplemented with all amino acids except methionine. All amino acids are added as a solution except for Tyrosine, Tryptophan and Phenylalanine which are added to the media in powder format. As well the media is supplemented with MgSO₄ (2 mM final concentration), FeSO₄.7H₂O (25 mg/L final concentration), Glucose (0.4% final concentration), CaCl₂ (0.1 mM final concentration) and Seleno-L-Methionine (40 mg/L final concentration). When the OD₆₀₀ of the cell culture reaches 0.8-0.9, IPTG (0.4 mM final concentration) is added to the medium for protein induction, and the cell culture is kept shaking at 15° C. for 10 hours. The cells are harvested by centrifugation at 3500 rpm at 4° C. for 20 minutes and the cell pellet is resuspended in 15 mL cold binding buffer (Hepes 50 mM, pH 7.5) and 100 μl of protease inhibitors (PMSF and Benzamidine) and flash frozen.

Alternatively, a starter culture is prepared in a 300 mL Tunair flask (Shelton Scientific) by adding 50 mL of sterile medium having 10% 10XM9 (37.4 mM NH₄Cl (Sigma; Cat. No. A4514), 44 mM KH₂PO₄ (Bioshop, Ontario, Canada; Cat. No. PPM 302), 96 mM Na₂HPO₄ (Sigma; Cat. No. S2429256), and 96 mM Na₂HPO₄.7H₂O (Sigma; Cat. No. S9390) final concentration), 450 μM alanine, 190 μM arginine, 302 μM asparagine, 300 μM aspartic acid, 330 μM cysteine, 272 μM glutamic acid, 274 μM glutamine, 533 μM glycine, 191 μM histidine, 305 μM isoleucine, 305 μM leucine, 220 μM lysine, 242 μM phenylalanine, 348 μM proline, 380 μM serine, 336 μM threonine, 196 μM tryptophan, 220 μM tyrosine, and 342 μM valine, 204 μM Seleno-L-Methionine (Sigma; Cat. No. S3132), 0.5% v/v of Kao and Michayluk vitamins mix (Sigma; Cat. No. K3129), 2 mM MgSO₄ (Sigma; Cat. No. M7774), 90 μM FeSO₄.7H₂O (Sigma; Cat. No. F8633), 0.4% glucose (Sigma; Cat. No. G-5400), 100 μM CaCl₂ (Bioshop, Ontario, Canada; Cat. No. CCL 302), 50 μg/mL Ampicillin, and 50 μg/mL Kanamycin in dH₂O. The medium is then inoculated with several colonies of E. coli B834 cells (Novagen) freshly transformed with an expression construct clone encoding the polypeptide of interest. The culture is then incubated at 37° C. and 200 rpm until it reaches an OD₆₀₀ of ˜1 and is then transferred to a 2.5 L Tunair Flask containing 1 L of the above media. The 1 L culture is incubated at 37° C. with shaking at 200 rpm until the culture reaches an OD₆₀₀ of 0.6-0.8 and is then induced with 0.5 mM IPTG. The induced culture is incubated overnight at 15° C. with shaking at 200 rpm. The cells are harvested by centrifugation at 4200 rpm at 4° C. for 20 minutes and the cell pellet is resuspended in 15 mL ice cold binding buffer (Hepes 50 mM, pH 7.5) and 100 μl of protease inhibitors (50 mM PMSF and 100 mM Benzamidine, stock concentration) and flash frozen.

Alternatively, the cell harboring a plasmid with the nucleic acid sequence of interest is inoculated into 10 ml of M9 minimum medium and kept shaking at 37° C. for 8-9 hours. This culture is then transferred into a 2 L Baffled Flask (Corning) containing 1 L minimum medium. The media is supplemented with all amino acids except methionine. All are added as a solution, except for Phenylalanine, Alanine, Valine, Leucine, Isoleucine, Proline, and Tryptophan which are added to the media in powder format. As well the media is supplemented with MgSO₄ (2 mM final concentrtion), FeSO₄.7H₂O (25 mg/L final concentration), Glucose (0.5% final concentration), CaCl₂ (0.1 mM final concentration) and Seleno-Methionine (50 mg/L final concentration). When the OD₆₀₀ of the cell culture reaches 0.8-0.9, IPTG (0.8 mM final concentration) is added to the medium for protein induction, and the cell culture is kept shaking at 25° C. for 4 hours. The cells are harvested by centrifuged at 3500 rpm at 4° C. for 20 minutes and the cell pellet is resuspended in 10 mL cold binding buffer (Hepes 50 mM, pH 7.5) and 100 μl of protease inhibitors (PMSF and Benzamidine) and flash frozen.

### Example 5

### Expression of ¹⁵N Labeled Polypeptides

The cell harboring a plasmid with the nucleic acid sequence of interest is inoculated into 2 L of minimal media (containing ¹⁵N isotope, Cambridge Isotope Lab) in a 6 L Erlenmeyer flask. The minimal media is supplemented with 0.01 mM ZnSO₄, 0.1 mM CaCl₂, 1 mM MgSO₄, 5 mg/L Thiamine.HCl, and 0.4% glucose. The 2 L culture is grown at 37° C. and 200 rpm to an OD₆₀₀ of between 0.7-0.8. The culture is then induced with 0.5 mM IPTG and allowed to shake at 15° C. for 14 hours. The cells are harvested by centrifugation and the cell pellet is resuspended in 15 mL cold binding buffer and 1001 μl of protease inhibitor and flash frozen. The protein is then purified as described below.

Alternatively, the cell, harboring a plasmid with the nucleic acid sequence of the invention, is inoculated into 10 mL of M9 media (with ¹⁵N isotope) and supplemented with 0.01 mM ZnSO₄, 0.1 mM CaCl₂, 1 mM MgSO₄, 5 mg/L Thiamine.HCl, and 0.4% glucose. After 8-10 hours of growth at 37° C., the culture is transferred to a 2 L Baffled flask (Corning) containing 990 mL of the same media. When OD₆₀₀ of the culture is between 0.7-0.8, protein production is initiated by adding IPTG to a final concentration of 0.8 mM and lowering the temperature to 25° C. After 4 hours of incubation at this temperature, the cells are harvested, and the cell pellet is resuspended in 10 mL cold binding buffer (Hepes 50 mM, pH 7.5) and 100 μl of protease inhibitor and flash frozen.

### Example 6

### Method One for Purifying Polypeptides of the Invention

The frozen pellets are thawed and sonicated to lyse the cells (5×30 seconds, output 4 to 5, 80% duty cycle, in a Branson Sonifier, VWR). The lysates are clarified by centrifugation at 14,000 rpm for 60 min at 4° C. to remove insoluble cellular debris. The supernatants are removed and supplemented with 1 μl of Benzonase Nuclease (25 U/μl, Novagen).

The recombinant protein is purified using DE52 (anion exchanger, Whatman) and Ni-NTA columns (Qiagen). The DE52 columns (30 mm wide, Biorad) are prepared by mixing 10 grams of DE52 resin in 25 ml of 2.5 M NaCl per protein sample, applying the resin to the column and equilibrating with 30 ml of binding buffer (50 mM in HEPES, pH 7.5, 5% glycerol (v/v), 0.5 M NaCl, 5 mM imidazole). Ni-NTA columns are prepared by adding 3.5-8 ml of resin to the column (20 mm wide, Biorad) based on the level of expression of the recombinant protein and equilibrating the column with 30 ml of binding buffer. The columns are arranged in tandem so that the protein sample is first passed over the DE52 column and then loaded directly onto the Ni-NTA column.

The Ni-NTA columns are washed with at least 150 ml of wash buffer (50 mM HEPES, pH 7.5, 5% glycerol (v/v), 0.5 M NaCl, 30 mM imidazole) per column. A pump may be used to load and/or wash the columns. The protein is eluted off of the Ni-NTA column using elution buffer (50 mM in HEPES, pH 7.5, 5% glycerol (v/v), 0.5 M NaCl, 250 mM imidazole) until no more protein is observed in the aliquots of eluate as measured using Bradford reagent (Biorad). The eluate is supplemented with 1 mM of EDTA and 0.2 mM DTT.

The samples are assayed by SDS-PAGE and stained with Coomassie Blue, with protein purity determined by visual staining.

Two methods may be used to remove the His tag located at either the C or N-terminus. In certain instances, the His tag may not be removed. In either case, the expressed polypeptide will have additional residues attributable to the His tag, as shown in the following table:

| | |
|---|---|
| SEQ ID NO | Type of Tag and |

| | | |
|---|---|---|
| for Additional Residues | Additional Residues | Whether or Not Removed |

| | | |
|---|---|---|
| | GSH | His tag removed from N- |
| | | terminus |
| SEQ ID NO: 1 | MGSSHHHHHHSSGLVPRGSH | His tag not removed from |
| | | N-terminus |
| SEQ ID NO: 2 | GSENLYFQGHHHHHH | His tag removed from C- |
| | | terminus |
| SEQ ID NO: 3 | GSENLYFQ | His tag not removed from |
| | | C-terminus |

In method one, a sample of purified polypeptide is supplemented with 2.5 mM CaCl₂ and an appropriate amount of thrombin (the amount added will vary depending on the activity of the enzyme preparation) and incubated for ˜20-30 minutes on ice in order to remove the His tag. In method two, a sample of purified polypeptide is combined with thirty units of recombinant TEV protease in 50 mmol TRIS HCl pH=8.0, 0.5 mmol EDTA and 1 mmol DTT, followed by incubation at 4° C. overnight, to remove the His tag.

The protein sample is then dialyzed in dialysis buffer (10 mM HEPES, pH 7.5, 5% glycerol (v/v) and 0.5 M NaCl) for at least 8 hours using a Slide-A-Lyzer (Pierce) appropriate for the molecular weight of the recombinant protein. An aliquot of the cleaved and dialyzed samples is then assayed by SDS-PAGE and stained with Coomassie Blue to determine the purity of the protein and the success of cleavage.

The remainder of the sample is centrifuged at 2700 rpm at 4° C. for 10-15 minutes to remove any precipitant and supplemented with 100 μl of protease inhibitor cocktail (0.1 M benzamidine and 0.05 M PMSF) (NO Bioshop). The protein is then applied to a second Ni-NTA column (˜8 ml of resin) to remove the His-tags and eluted with binding buffer or wash buffer until no more protein is eluting off the column as assayed using the Bradford reagent. The eluted sample is supplemented with 1 mM EDTA and 0.6 mM of DTT and concentrated to a final volume of ˜15 mls using a Millipore Concentrator with an appropriately sized filter at 2700 rpm at 4° C. The samples are then dialyzed overnight against crystallization buffer and concentrated to final volume of 0.3-0.7 ml.

### Example 7

### Method Two for Purifying Polypeptides of the Invention

The frozen pellets are thawed and supplemented with 100 μl of protease inhibitor (0.1 M benzamidine and 0.05 M PMSF), 0.5% CHAPS, and 4 U/ml Benzonase Nuclease. The sample is then gently rocked on a Nutator (VWR, setting 3) at room temperature for 30 minutes. The cells are then lysed by sonication (1×30 seconds, output 4 to 5, 80% duty cycle, in a Branson Sonifier, VWR) and an aliquot is saved for a gel sample.

The recombinant protein is purified using a three column system. The columns are set up in tandem so that the lysate flows from a Biorad Econo (5.0×30 cm×589 ml) “lysate” column onto a Biorad Econo (2.5×20 cm×98 ml) DE52 column and finally onto a Biorad Econo (1.5×15 cm×27 ml) Ni-NTA column; The lysate is mixed with 10 g of equilibrated DE52 resin and diluted to a total volume of 300 ml with binding buffer. This mixture is poured into the first column which is empty. The remainder of the purification procedure is described in EXAMPLE 6 above.

### Example 8

### Method Three for Purifying Polypeptides of the Invention

The frozen pellets are thawed and sonicated to lyse the cells (5×30 seconds, output 4 to 5, 80% duty cycle, in a Branson Sonifier, VWR). The lysates are clarified by centrifugation at 14000 rpm for 60 min at 4° C. to remove insoluble cellular debris. The supernatants are removed and supplemented with 1 μl of Benzonase Nuclease (25 U/μl, Novagen).

The recombinant protein is purified using DE52 (anion exchanger, Whatman) and Glutathione sepharose columns (Glutathione-Superflow resin, Clontech). The DE52 columns (30 mm wide, Biorad) are prepared by mixing 10 grams of DE52 resin in 20 ml of 2.5 M NaCl per protein sample, applying the resin to the column and equilibrating with 30 ml of loading buffer (50 mM in HEPES, pH 7.5, 10% glycerol (v/v), 0.5 M NaCl, 1 mM EDTA, 1 mM DTT). Glutathione sepharose columns are prepared by adding 3 ml of resin to the column (20 mm wide, Biorad) and equilibrating the column with 30 ml of loading buffer. The columns are arranged in tandem so that the protein sample is first passed over the DE52 column and then loads directly onto the Glutathione sepharose column.

The columns are washed with at least 150 ml of loading buffer supplemented with protease inhibitor cocktail (0.1 M benzamidine and 0.05 M PMSF) per column. A pump may be used to load and/or wash the columns. The protein is eluted off of the Glutathione sepharose column using elution buffer (20 mM HEPES, pH 7.5, 0.5 M NaCl, 1 mM EDTA, 1 mM DTT; 25 mM glutathione (reduced form)) until no more protein is observed in the aliquots of eluate as measured using Biorad Bradford reagent.

The GST tag may be removed using thrombin or other procedures known in the art. The protein samples are then dialyzed into crystallization buffer (10 mM Hepes, pH 7.5, 500 mM NaCl) to remove free glutathione and assayed by SDS-PAGE followed by staining with Coomassie blue. Prior to use or storage, the samples are concentrated to final volume of 0.3-0.5 ml.

The Tables contained in the Figures set forth the results of expressing and purifying certain of the polypeptides of the invention using, the procedures described above. Prepared and purified in this way, the purified polypeptides are essentially the only protein visualized in the SDS-PAGE assay using Coomassie Blue described above, which is at least about 95% or greater purity.

The protein samples so prepared and purified may be used in the studies that follow and that are otherwise described herein, with or without the tag or the residual amino acids resulting from removal of the tag. In certain instances, such as EXAMPLE 11, the polypeptide sample used may be a fusion protein with a specific tag.

A stable solution of certain of the expressed polypeptides, labeled and unlabeled, tagged and untagged, may be prepared in one ml of either the dialysis or crystallization buffers (or possibly both) described above in EXAMPLE 6 or EXAMPLE 8. The results of those solubility experiements are set forth in the applicable Table contained in the Figures.

For certain polypeptides of the invention, truncated polypeptides are prepared. Truncated polypeptides are generated via a “shot gun” approach whereby 1 to about 15 or more residues may be deleted from the N and/or C termini of the polypeptide of interest in a sequential pattern, in a variety of combinations of deletions. Alternatively, truncated polypeptides may be prepared by rational design, using multiple sequence alignments of the protein and other orthologues, secondary structure prediction and tertiary structure of a related protein (if available) as guiding tools. In such cases, from 1 to about 20 amino acids or more may be deleted from the N and/or C termini. Truncated constructs are PCR amplified from genomic DNA and cloned into expression vectors as described above for the various pathogens. Truncation constructs are then tested for expression and solubility as described above. The most highly expressed and soluble truncated polypeptides may be subject to further purification and characterization as provided herein.

### Example 9

### Mass Spectrometry Analysis via Fingerprint Mapping

A gel slice from a purification protocol described above containing a polypeptide of the invention is \cut into 1 mm cubes and 10 to 20 μl of 1% acetic acid is added. After washing with 100-150 μl HPLC grade water and removal of the liquid, acetonitrile (˜200 μl, approximately 3 to 4 times the volume of the gel particles) is added followed by incubation at room temperature for 10 to 15 minutes with vortexing. A second acetonitrile wash may be required to completely dehydrate the gel particles. The protein in the gel particles is reduced at 50 degrees Celsius using 10 mM dithiothreitol (in 100 mM ammonium bicarbonate) and then alkylated at room temperature in the dark using 55 mM iodoacetamide (in 100 mM ammonium bicarbonate). The gel particles are rinsed with a minimal volume of 100 mM ammonium bicarbonate before a trypsin (50 mM ammonium bicarbonate, 5 mM CaCl₂, and 12.5 ng/μl trypsin) solution is added. The gel particles are left on ice for 30 to 45 minutes (after 20 minutes incubation more trypsin solution is added). The excess trypsin solution is removed and 10 to 15 μl digestion buffer without trypsin is added to ensure the gel particles remain hydrated during digestion. After digestion at 37° C., the supernatant is removed from the gel particles. The peptides are extracted from the gel particles with 2 changes of 100 μL of 100 mM ammonium bicarbonate with shaking for 45 minutes and pooled with the initial gel supernatant. The extracts are acidified to 1% (v/v) with 100% acetic acid.

The tryptic peptides are purified with a C18 reverse phase resin. 250 μL of dry resin is washed twice with methanol and twice with 75% acetonitrile/1% acetic acid. A 5:1 slurry of solvent:resin is prepared with 75% acetonitrile/1% acetic acid. To the extracted peptides, 2 μL of the resin slurry is added and the solution is shaken for 30 minutes at room temperature. The supernatant is removed and replaced with 200 μL of 2% acetonitrile/1% acetic acid and shaken for 5-15 minutes. The supernatant is removed and the peptides are eluted from the resin with 15 μL of 75% acetonitrile/1% acetic acid with shaking for about 5 minutes. The peptide and slurry mixture is applied to a filter plate and centrifuged, and the filtrate is collected and stored at −70° C. until use.

Alternatively, the tryptic peptides are purified using ZipTipc₁₈ (Millipore, Cat # ZTC18S960). The ZipTips are first pre-wetted by aspirating and dispensing 100% methanol. The tips are then washed with 2% acetonitrile/1% acetic acid (5 times), followed by 65% acetonitrile/1% acetic (5 times) and returned to 2% acetonitrile/1% acetic acid (10 times). The digested peptides are bound to the ZipTips by aspirating and dispensing the samples 5 times. Salts are removed by washing ZipTips with 2% acetonitrile/1% acetic acid (5 times). 10 μL of 65% acetonitrile/1% acetic acid is collected by the ZipTips and dispensed into a 96-well microtitre plate.

Analytical samples containing tryptic peptides are subjected to MALDI-TOF mass spectrometry. Samples are mixed 1:1 with a matrix of α-cyano-4-hydroxy-trans-cinnamic acid. The sample/matrix mixture is spotted on to the MALDI sample plate with a robot, either a Gilson 215 liquid handler or BioMek FX laboratory automation workstation (Beckman). The sample/matrix mixture is allowed to dry on the plate and is then introduced into the mass spectrometer. Analysis of the peptides in the mass spectrometer is conducted using both delayed extraction mode (400 ns delay) and an ion reflector to ensure high resolution of the peptides.

Internally-calibrated tryptic peptide masses are searched against databases using a correlative mass matching algorithm. The Proteometrics software package (ProteoMetrics) is utilized for batch database searching of tryptic peptide mass spectra Statistical analysis is performed on each protein match to determine its validity. Typical search constraints include error tolerances within 0.1 Da for monoisotopic peptide masses, carboxyamidomethylation of cysteines, no oxidation of methionines allowed, and 0 or 1 missed enzyme cleavages. The software calculates the probability that a candidate in the database search is the protein being analyzed, which is expressed as the Z-score. The Z-score is the distance to the population mean in unit of standard deviation and corresponds to the percentile of the search in the random match population. If a search is in the 95th percentile, for example, about 5% of random matches could yield a higher Z-score than the search. A Z-score of 1.282 for a search indicates that the search is in the 90th percentile, a Z-score of 1.645 indicates that the search is in the 95th percentile, a Z-score of 2.326 indicates that the search is in the 99th percentile, and a Z-score of 3.090 indicates that the search is in the 99.9th percentile.

The results of the mass search described above for certain of the polypeptides of the invention are shown in the Figures, and described in the applicable Table contained in the Figures, for each of them. From these experiments, the identity of those polypeptides have been confirmed.

### Example 10

### Mass Spectrometry Analysis via High Mass

A matrix solution of 25 mg/mL of 3,5-dimethoxy-4-hydroxycinnamic acid (sinapinic acid) in 66% (v/v) acetonitrile/1% (v/v) acetic acid is prepared along with an internal calibrant of carbonic anhydrase. On to a stainless steel polished MALDI target, 1.5 μL of a protein solution (concentration of 2 μg/μL) is spotted, followed immediately by 1.5 μL of matrix. 3 μL of 40% (v/v) acetonitrile/1% (v/v) acetic acid is then added to each spot has dried. The sample is either spotted manually or utilizing a Gilson 215 liquid handler or BioMek FX laboratory automation workstation (Beckman). The MALDI-TOF instrument utilizes positive ion and linear detection modes. Spectra are acquired automatically over a mass to charge range from 0-150,000 Da, pulsed ion extraction delay is set at 200 ns, and 600 summed shots of 50-shot steps are completed.

The theoretical molecular weight of the protein for MALDI-TOF is determined from its amino acid sequence, taking into account any purification tag or residue thereof still present and any labels (e.g., selenomethionine or ¹⁵N). To account for ¹⁵N incorporation, an amount equal to the theoretical molecular weight of the protein divided by 70 is added. The mass of water is subtracted from the overall molecular weight. The MALDI-TOF spectrum is calibrated with the internal calibrant of carbonic anhydrase (observed as either [MH⁺_{avg}] 29025 or [MH₂²⁺] 14513).

One or more of the Figures display the MALDI-TOF-generated mass spectrum of certain of the polypeptides of the present invention.

The calculated molecular weight, and the experimentally determined molecular weight, for certain polypeptides of the invention are listed in the applicable Table contained in the Figures. In certain instances, a lower mass to charge peak may also be present, which signifies the presence of doubly-charged molecular ion peak [MH₂²⁺] of the polypeptide,

### Example 11

### Method One for Isolating and Identifying Interacting Proteins

(a) Method One for Preparation of Affinity Column

Micro-columns are prepared using forceps to bend the ends of P200 pipette tips and adding 10 μl of glass beads to act as a column frit. Six micro-columns are required for every polypeptide to be studied. The micro-columns are placed in a 96-well plate that has 1 mL wells. Next, a series of solutions of a polypeptide comprising a subject amino acid sequence (experimental), prepared and purified as described above and with a GST tag on either terminus, is prepared so as to give final amounts of 0, 0.1, 0.5, 1.0, and 2.0 mg of ligand per ml of resin volume.

A slurry of Glutathione-Sepharose 4B (Amersham) is prepared and 0.5 ml slurry/ligand is removed (enough for six 40-μg aliquots of resin). Using a glass frit Buchner funnel, the resin is washed sequentially with three 10 ml portions each of distilled H₂O and 1 M ACB (20 mM HEPES pH 7.9, 1 M NaCl, 10% glycerol, 1 mM DTT, and 1 mM EDTA). The Glutathione-Sepharose 4B is completely drained of buffer, but not dried. The Glutathione-Sepharose 4B is resuspended as a 50% slurry in 1 M ACB and 80 μl is added to each micro-column to obtain 40 μg/column. The buffer containing the ligand concentration series is added to the columns and allowed to flow by gravity. The resin and ligand are allowed to cross-link overnight at 4° C. In the morning, micro-columns are washed with 100 μl of 1 M ACB and allowed to flow by gravity. This is repeated twice more and the elutions are tested for cross-linking efficiency by measuring the amount of unbound ligand. After washing, the micro-columns are equilibrated using 200 μl of 0.1 M ACB (20 mM HEPES pH 7.5, 0.1 M NaCl, 10% glycerol, 1 mM DTT, 1 mM EDTA).

In another method, the recombinant GST fusion protein can be replaced by a hexa-histidine fusion peptide for use with NTA-Agarose (Qiagen) as the solid support. No adaptation to the above protocol is required for the substitution of NTA agarose for GST Sepharose except that the recombinant protein requires a six histidine fusion peptide in place of the GST fusion.

(b) Method Two for Preparation of Affinity Column

In an alternative method, GST-Sepharose 4B may be replaced by Affi-gel 10 Gel (Bio-Rad). The column resin for affinity chromatography could also be Affigel 10 resin which allows for covalent attachment of the protein ligand to the micro affinity column. An adaptation to the above protocol for the use of this resin is a pre-wash of the resin with 100% isopropanol. No fusion peptides or proteins are required for the use of Affigel 10′ resin.

(c) Method One for Bacterial Extract Preparation

A S. aureus extract is prepared from cell pellets using nuclease and lysostaphin digestion followed by sonication. A S. aureus cell pellet (12 g) is suspended in 12 ml of 20 mM HEPES pH 7.5, 150 mM NaCl, 10% glycerol, 10 mM MgSO₄, 10 mM CaCl₂, 1 mM DTT, 1 mM PMSF, 1 mM benzamidine, 1000 units of lysostaphin, 0.5 mg RNAse A, 750 units micrococcal nuclease, and 375 units DNAse I. The cell suspension is incubated at 37° C. for 30 minutes, cooled to 4° C., and brought to a final concentration of 1 mM EDTA and 500 mM NaCl. The lysate is sonicated on ice using three bursts of 20 seconds each. The lysate is centrifuged at 20,000 rpm for 1 hr in a Ti70 fixed angle Beckman rotor. The supernatant is removed and dialyzed overnight in a 10,000 Mr dialysis membrane against dialysis buffer (20 mM HEPES pH 7.5, 10% glycerol, 1 mM DTT, 1 mM EDTA, 100 mM NaCl, 10 mM MgSO₄, 10 mM CaCl₂, 1 mM benzamidine, and 1 mM PMSF). The dialyzed protein extract is removed from the dialysis tubing and frozen in one ml aliquots at −70° C.

An E. coli extract is prepared from cell pellets using a French press followed by sonication. An E. coli cell pellet (˜6 g) is suspended in 3 pellet volumes (˜20 ml final volume) of 20 mM HBPESpH 7.5, 150 mM NaCl, 10% glycerol, 10 mM MgSO₄, 10 mM CaCl₂, 1 mM DTT, 1 mM PMSF, 1 mM benzamidine, 40 μg/ml RNAse A, 75 units/ml S1 nuclease, and 40 units/ml DNAse 1. The cell suspension is lysed with one pass with a French Pressure Cell followed by sonication on ice using three bursts of 20 seconds each. The lysate is agitated at 4° C. for 30 minutes, brought up to 0.5 M NaCl and then incubated for an additional 30 min at 4° C. with agitation. The lysate is centrifuged at 25,000 rpm for 1 hr at 4° C. in a Ti70 fixed angle Beckman rotor. The supernatant is removed and dialyzed overnight in a 10,000 Mr dialysis membrane against dialysis buffer (20 mM HEPES pH 7.5, 10% glycerol, 1 mM DTT, 1 mM EDTA, 10 mM MgSO₄, 10 mM CaCl₂, 100 mM NaCl, 1 mM benzamidine, and 1 mM PMSF). The dialyzed protein extract is removed from the dialysis tubing and frozen in one ml aliquots at −70° C.

An H. pylori extract is prepared from cell pellets using a French press followed by sonication. An H. pylori cell pellet (˜6 g) is suspended in 3 pellet volumes (˜20 ml final volume) of 20 mM HEPES pH 7.5, 150 mM NaCl, 10% glycerol, 10 mM MgSO₄, 10 mM CaCl₂, 1 mM DTT, 1 mM PMSF, 1 mM benzamidine, 40 μg/ml RNAse A, 75 units/ml S1 nuclease, and 40 units/ml DNAse 1. The cell suspension is lysed with one pass with a French Pressure Cell followed by sonication on ice using three bursts of 20 seconds each. The lysate is agitated at 4° C. for 30 minutes, brought up to 0.5 M NaCl and then incubated for an additional 30 min at 4° C. with agitation. The lysate is centrifuged at 25,000 rpm for 1 hr at 4° C. in a Ti70 fixed angle Beckman rotor. The supernatant is removed and dialyzed overnight in a 10,000 Mr dialysis membrane against dialysis buffer (20 mM HEPES pH 7.5, 10% glycerol, 1 mM DTT, 1 mM EDTA, 100 mM NaCl, 10 mM MgSO₄, 10 mM CaCl₂, 1 mM benzamidine, and 1 mM PMSF). The dialyzed protein extract is removed from the dialysis tubing and frozen in one ml aliquots at −70° C.

A P. aeruginosa extract is prepared from cell pellets using a French press followed by sonication. An P. aeruginosa cell pellet (˜6 g) is suspended in 3 pellet volumes (˜20 ml final volume) of 20 mM HEPES pH 7.5, 150 mM NaCl, 10% glycerol, 10 mM MgSO₄, 10 mM CaCl₂, 1 mM DTT, 1 mM PMSF, 1 mM benzamidine, 40 μg/ml RNAse A, 75 units/ml S1 nuclease, and 40 units/ml DNAse 1. The cell suspension is lysed with one pass with a French Pressure Cell followed by sonication on ice using three bursts of 20 seconds each. The lysate is agitated at 4° C. for 30 minutes, brought up to 0.5 M NaCl and then incubated for an additional 30 min at 4° C. with agitation. The lysate is centrifuged at 25,000 rpm for 1 hr at 4° C. in a Ti70 fixed angle Beckman rotor. The supernatant is removed and dialyzed overnight in a 10,000 Mr dialysis membrane against dialysis buffer (20 mM HEPES pH 7.5, 10% glycerol, 1 mM DTT, 1 mM EDTA, 100 mM NaCl, 10 mM MgSO₄, 10 mM CaCl₂, 1 mM benzamidine, and 1 mM PMSF). The dialyzed protein extract is removed from the dialysis tubing and frozen in one ml aliquots at −70° C.

A S. pneumoniae extract is prepared from cell pellets using a French press followed by sonication. An S. pneumoniae cell pellet (˜6 g) is suspended in 3 pellet volumes (˜20 ml final volume) of 20 mM HEPES pH 7.5, 150 mM NaCl, 10% glycerol, 10 mM MgSO₄, 10 mM CaCl₂, 1 mM DTT, 1 mM PMSF, 1 mM benzamidine, 40 μg/ml RNAse A, 75 units/ml S1 nuclease, and 40 units/ml DNAse 1. The cell suspension is lysed with one pass with a French Pressure Cell followed by sonication on ice using three bursts of 20 seconds each. The lysate is agitated at 4° C. for 30 minutes, brought up to 0.5 M NaCl and then incubated for an additional 30 min at 4° C. with agitation. The lysate is centrifuged at 25,000 rpm for 1 hr at 4° C. in a Ti70 fixed angle Beckman rotor. The supernatant is removed and dialyzed overnight in a 10,000. Mr dialysis membrane against dialysis buffer (20 mM HEPES pH 7.5, 10% glycerol, 1 mM DTT, 1 mM EDTA, 100 mM NaCl, 10 mM MgSO₄, 10 mM CaCl₂, 1 mM benzamidine, and 1 mM PMSF). The dialyzed protein extract is removed from the dialysis tubing and frozen in one ml aliquots at −70° C.

An E. faecalis extract is prepared from cell pellets using a French press followed by sonication. An E. faecalis cell pellet (˜6 g) is suspended in 3 pellet volumes (˜20 ml final volume) of 20 mM HEPES pH 7.5, 150 mM NaCl, 10% glycerol, 10 mM MgSO₄, 10 mM CaCl₂, 1 mM DTT, 1 mM PMSF, 1 mM benzamidine, 40 μg/ml RNAse A, 75 units/ml S1 nuclease, and 40 units/ml DNAse 1. The cell suspension is lysed with one pass with a French Pressure Cell followed by sonication on ice using three bursts of 20 seconds each. The lysate is agitated at 4° C. for 30 minutes, brought up to 0.5 M NaCl and then incubated for an additional 30 min at 4° C. with agitation. The lysate is centrifuged at 20,000 rpm for 1 hr in a JA25.50 Beckman rotor. The supernatant is removed and dialyzed overnight in a 3,500 Mr dialysis membrane against dialysis buffer (20 mM HEPES pH 7.5, 10% glycerol, 1 mM DTT, 1 mM EDTA, 100 mM NaCl, 10 mM MgSO₄, 10 mM CaCl₂, 1 mM benzamidine, and 1 mM PMSF). The dialyzed protein extract is removed from the dialysis tubing and frozen in one ml aliquots at −70° C.

(d) Method Two for Bacterial Extract Preparation

Bacterial cell extracts from the pathogen of interest are prepared from cell pellets using a Bead-Beater apparatus (Bio-spec Products Inc.) and zirconia beads (0.1 mm diameter). The bacterial cell pellet is suspended (˜6 g) is suspended in 3 pellet volumes (˜20 ml final volume) of 20 mM HEPES pH 7.5, 150 mM NaCl, 10% glycerol, 10 mM MgSO₄, 10 mM CaCl₂, 1 mM DTT, 1 mM PMSF, 1 mM benzamidine, 40 μg/ml RNAse A, 75 units/ml S1 nuclease, and 40 units/ml DNAse 1. The cells are lysed with 10 pulses of 30 sec between 90 sec pauses at a temperature of −5° C. The lysate is separated from the zirconia beads using a standard column apparatus. The lysate is centrifuged at 20000 rpm (48000×g) in a Beckman JA25.50 rotor. The supernatant is removed and dialyzed overnight at 4° C. against dialysis buffer (20 mM HEPES pH 7.5, 10% glycerol, 1 mM DTT, 1 mM EDTA, 100 mM NaCl, 10 mM MgSO₄, 10 mM CaCl₂, 1 mM benzamidine, and 1 mM PMSF). The dialyzed protein extract is removed from the dialysis tubing and frozen in one ml aliquots at −70° C.

(e) HeLa Cell Extract Preparation

A HeLa cell extract is prepared in the presence of protease inhibitors. Approximately 30 g of Hela cells are submitted to a freeze/thaw cycle and then divided into two tubes. To each tube 20 ml of Buffer A (10 mM HEPES pH 7.9, 1.5 mM MgCl, 10 mM KCl, 0.5 mM DTT, 0.5 mM PMSF) and a protease inhibitor cocktail are added. The cell suspension is homogenized with 10 strokes (2×5 strokes) to lyse the cells. Buffer B (15 ml per tube) is added (50 mM HEPES pH 7.9, 1.5 mM MgCl, 1.26 M NaCl, 0.5 mM DTT, 0.5 mM PMSF, 0.5 mM EDTA, 75% glycerol) to each tube followed by a second round of homogenization (2×5 strokes). The lysates are stirred on ice for 30 minutes followed by centrifugation 37,000 rpm for 3 hr at 4° C. in a Ti70 fixed angle Beckman rotor. The supernatant is removed and dialyzed overnight in a 10,000 Mr dialysis membrane against dialysis buffer (20 mM HEPES pH 7.9, 10% glycerol, 1 mM DTT, 1 mM EDTA, and 1 M NaCl. The dialyzed protein extract is removed from the dialysis tubing and frozen in one ml aliquots at −70° C.

(f) Affinity Chromatography

Cell extract is thawed and diluted to 5 mg/ml prior to loading 5 column volumes onto each micro-column. Each column is washed with 5 column volumes of 0.1 M ACB. This washing is repeated once. Each column is then washed with 5 column volumes of 0.1 M ACB containing 0.1% Triton X-100. The columns are eluted with 4 column volumes of 1% sodium dodecyl sulfate into a 96 well PCR plate. To each eluted fraction is added one-tenth volume of 10-fold concentrated loading buffer for SDS-PAGE.

(g) Resolution of the Eluted Proteins and Detection of Bound Proteins

The components of the eluted samples are resolved on SDS-polyacrylamide gels containing 13.8% polyacrylamide using the Laemmli buffer system and stained with silver nitrate. The bands containing the interacting protein are excised with a clean scalpel. The gel volume is kept to a minimum by cutting as close to the band as possible. The gel slice is placed into one well of a low protein binding, 96-well round-bottom plate. To the gel slices is added 20 μl of 1% acetic acid.

### Example 12

### Method Two for Isolating and Identifying Interacting Proteins

Interacting proteins may be isolated using immunoprecipitation. Naturally-occurring bacterial or eukaryotic cells are grown in defined growth conditions or the cells can be genetically manipulated with a protein expression vector. The protein expression vector is used to transiently transfect the cDNA of interest into eukaryotic or prokaryotic cells and the protein is expressed for up to 24 or 48 hours. The cells are harvested and washed three times in sterile 20 mM HEPES (pH7.4)/Hanks balanced salts solution (H/H). The cells are finally resuspended in culture media and incubated at 37° C. for 4-8 hr.

The harvested cells may be subjected to one or more culture conditions that may alter the protein profile of the cells for a given period of time. The cells are collected and washed with ice-cold H/H that includes 10 mM sodium pyrophosphate, 10 mM sodium fluoride, 10 mM EDTA, and 1 mM sodium orthovanadate. The cells are then lysed in lysis buffer (50 mM Tris-HCl (pH 8.0), 150 mM NaCl, 1% Triton X-100, 10 mM sodium pyrophosphate, 10 mM sodium fluoride, 10 mM EDTA, 1 mM sodium orthovanadate, 1 μg/mL PMSF, 1 g/mL aprotinin, 1 μg/mL leupeptin, and 1 μg/mL pepstatin A) by gentle mixing, and placed on ice for 5 minutes. After lysis, the lysate is transferred to centrifuge tubes and centrifuged in an ultracentrifuge at 75000 rpm for 15 min at 4° C. The supernatant is transferred to eppendorf tubes and pre-cleared with 10 μl of rabbit pre-immune antibody on a rotator at 4° C. for 1 hr. Forty μl of protein A-Sepharose (Amersham) is then added and incubated at 4° C. overnight on a rotator.

The protein A-Sepharose beads are harvested and the supernatant removed to a fresh eppendorf tube. Immune antibody is added to supernatant and rotated for 1 hr at 4° C. Thirty μl of protein A-Sepharose is then added and the mixture is further rotated at 4° C. for 1 hr. The beads are harvested and the supernatant is aspirated. The beads are washed three times with 50 mM Tris (pH 8.0), 150 mM NaCl, 0.1% Triton X-100, 10 mM sodium fluoride, 10 mM sodium pyrophosphate, 10 mM sodium orthovanadate, and 10 mM EDTA. Dry the beads with a 50 μl Hamilton syringe. Laemmli loading buffer containing 100 mM DTT is added to the beads and samples are boiled for 5 min. The beads are spun down and the supernatant is loaded onto SDS-PAGE gels. Comparison of the control and experimental samples allows for the selection of polypeptides that interact with the protein of interest.

### Example 13

### Sample for Mass Spectrometry of Interacting Proteins

The gel slices are cut into 1 mm cubes and 10 to 20 μl of 1% acetic acid is added. The gel particles are washed with 100-150 μl of HPLC grade water (5 minutes with occasional mixing), briefly centrifuged, and the liquid is removed. Acetonitrile (˜200 μl, approximately 3 to 4 times the volume of the gel particles) is added followed by incubation at room temperature for 10 to 15 minutes with vortexing. A second acetonitrile wash may be required to completely dehydrate the gel particles. The sample is briefly centrifuged and all the liquid is removed.

The protein in the gel particles is reduced at 50 degrees Celsius using 10 mM dithiothreitol (in 100 mM ammonium bicarbonate) for 30 minutes and then alkylated at room temperature in the dark using 55 mM iodoacetamide (in 100 mM ammonium bicarbonate). The gel particles are rinsed with a minimal volume of 100 mM ammonium bicarbonate before a trypsin (50 mM ammonium bicarbonate, 5 mM CaCl₂, and 12.5 ng/μl trypsin) solution is added. The gel particles are left on ice for 30 to 45 minutes (after 20 minutes incubation more trypsin solution is added). The excess trypsin solution is removed and 10 to 15 μl digestion buffer without trypsin is added to ensure the gel particles remain hydrated during digestion. The samples are digested overnight at 37° C.

The following day, the supernatant is removed from the gel particles. The peptides are extracted from the gel particles with 2 changes of 100 μL of 100 mM ammonium bicarbonate with shaking for 45 minutes and pooled with the initial gel supernatant. The extracts are acidified to 1% (v/v) with 100% acetic acid.

(a) Method One for Purification of Tryptic Peptides

The tryptic peptides are purified with a C18 reverse phase resin. 250 μL of dry resin is washed twice with methanol and twice with 75% acetonitrile/1% acetic acid. A 5:1 slurry of solvent:resin is prepared with 75% acetonitrile/1% acetic acid. To the extracted peptides, 2 μL of the resin slurry is added and the solution is shaken at moderate speed for 30 minutes at room temperature. The supernatant is removed and replaced with 200 μL of 2% acetonitrile/1% acetic acid and shaken for 5-15 minutes with moderate speed. The supernatant is removed and the peptides are eluted from the resin with 15 μL of 75% acetonitrile/1% acetic acid with shaking for about 5 minutes. The peptide and slurry mixture is applied to a filter plate and centrifuged for 1-2 minutes at 1000 rpm, the filtrate is collected and stored at −70° C. until use.

(b) Method Two for Purification of Tryptic Peptides

Alternatively, the tryptic peptides may be purified using ZipTip_{C18} (Millipore, Cat # ZTC18S960). The ZipTips are first pre-wetted by aspirating and dispensing 100% methanol 5 times. The tips are then washed with 2% acetonitrile/1% acetic acid (5 times), followed by 65% acetonitrile/1% acetic (5 times) and returned to 2% acetonitrile/1% acetic acid (5 times). The ZipTips are replaced in their rack and the residual solvent is eliminated. The ZipTips are washed again with 2% acetonitrile/1% acetic acid (5 times). The digested peptides are bound to the ZipTips by aspirating and dispensing the samples 5 times. Salts are removed by washing ZipTips with 2% acetonitrile/1% acetic acid (5 times). 10 μL of 65% acetonitrile/1% acetic acid is collected by the ZipTips and dispensed into a 96-well microtitire plate. 1 μL of sample and 1 μL of matrix are spotted on a MALDI-TOF sample plate for analysis.

### Example 14

### Mass Spectrometric Analysis of Interacting Proteins

(a) Method One for Analysis of Tryptic Peptides

Analytical samples containing tryptic peptides are subjected to Matrix Assisted Laser Desorption/Ionization Time Of Flight (MALDI-TOF) mass spectrometry. Samples are mixed 1:1 with a matrix of α-cyano-4-hydroxy-trans-cinnamic acid. The sample/matrix mixture is spotted on to the MALDI sample plate with a robot. The sample/matrix mixture is allowed to dry on the plate and is then introduced into the mass spectrometer. Analysis of the peptides in the mass spectrometer is conducted using both delayed extraction mode and an ion reflector to ensure high resolution of the peptides.

Internally-calibrated tryptic peptide masses are searched against both in-house proprietary and public databases using a correlative mass matching algorithm. Statistical analysis is performed on each protein match to determine its validity. Typical search constraints include error tolerances within 0.1 Da for monoisotopic peptide masses and carboxyamidomethylation of cysteines. Identified proteins are stored automatically in a relational database with software links to SDS-PAGE images and ligand sequences.

(b) Method Two for Analysis of Tryptic Peptides

Alternatively, samples containing tryptic peptides are analyzed with an ion trap instrument. The peptide extracts are first dried down to approximately 1 μL of liquid. To this, 0.1% trifluoroacetic acid (TFA) is added to make a total volume of approximately 5 μL. Approximately 1-2 μL of sample are injected onto a capillary column (C8, 150 μm ID, 15 cm long) and run at a flow rate of 800 nL/min. using the following gradient program:

| Time (minutes) | % Solvent A | % Solvent B |
|---|---|---|
| 0 | 95 | 5 |
| 30 | 65 | 35 |
| 40 | 20 | 80 |
| 41 | 95 | 5 |

Where Solvent A is composed of water/0.5% acetic acid and Solvent B is acetonitrile/0.5% acetic acid. The majority of the peptides will elute between the 20-40% acetonitrile gradient. Two types of data from the eluting HPLC peaks are acquired with the ion trap mass spectrometer. In the MS¹ dimension, the mass to charge range for scanning is set at 400-1400—this will determine the parent ion spectrum. Secondly, the instrument has MS² capabilities whereby it will acquire fragmentation spectra of any parent ions whose intensities are detected to be greater than a predetermined threshold Mann and Wilm, Anal Chem 66(24): 4390-4399 (1994)). A significant amount of information is collected for each protein sample as both a parent ion spectrum and many daughter ion spectra are generated with this instrumentation.

All resulting mass spectra are submitted to a database search algorithm for protein identification. A correlative mass algorithm is utilized along with a statistical verification of each match to identify a protein's identification (Ducret A, et al., Protein Sci 7(3): 706-719 (1998)). This method proves much more robust than MALDI-TOF mass spectrometry for identifying the components of complex mixtures of proteins.

The results of the interaction studies for certain of the subject polypeptides are set forth in the applicable Table contained in the Figures.

### Example 15

### NMR Analysis

Purified protein sample is centrifuged at 13,000 rpm for 10 minutes with a bench-top microcentrifuge to eliminate any precipitated protein. The supernatant is then transferred into a clean tube and the sample volume is measured. If the sample volume is less than 450 μl, an appropriate amount of crystal buffer is added to the sample to reach that volume. Then 50 μl of D₂O (99.9%) is added to the sample to make an NMR sample of 500 μl. The usual concentration of the protein sample is usually approximately 1 mmol or greater.

NMR screening experiments are performed on a Bruker AV600 spectrometer equipped with a cryoprobe, or other equivalent instrumentation. All spectra are recorded at 25° C. Standard 1D proton pulse sequence with presaturation is used for 1D screening. Normally, a sweepwidth of 6400 Hz, and eight or sixteen scans are used, although different pulse sequences are known to those of skill in the art and may be readily determined. For ¹H, ¹⁵N HSQC experiments, a pulse sequence with “flip-back” water suppression may be used. Typically, sweepwidths of 8000 Hz and 2000 Hz are used for F2 and F1 dimension, respectively. Four to sixteen scans are normally adequate. The data is then processed on a Sun Ultra 5 computer with NMRpipe software.

One or more representative NMR spectra from a ¹H, ¹⁵N HSQC experiment generated with certain polypeptides of the invention, prepared and purified as described above, are presented in the Figures.

### Example 16

### X-Ray Crystallography

(a) Crystallization

Subsequent to purification, a subject polypeptide is centrifuged for 10 minutes at 4° C. and at 14,000 rpm in order to sediment any aggregated protein. The protein sample is then diluted in order to provide multiple concentrations for screening.

Two 96 well plates (Nunc) are employed for the initial crystal screen, with 48 potential crystallization conditions. The screening library has crystallization conditions found in Hampton Research Crystal Screen I (Jankarik, J. and S. H. Kim, J. Appl. Cryst., 1991. 24:409-11), Hampton Research Crystal Screen II, Hampton Crystal Screen I-Lite, and from Emerald Biostructures, Inc., Bainbridge Island, Wash., Wizard I, Wizard II, Cryo I and Cryo II. Alternatively, other conditions known to those of skill in the art, including those provided in screening kits available from other companies, may also be tested.

Conditions are tested at multiple protein concentrations and at two temperatures (4 and 20° C.). Crystal setups may be performed by a liquid handling robot appropriately programmed for sitting drop experiments. The robot loads 50 μl of buffer into each screening well on a 24 or 96 well sitting drop crystal screen tray, and then loads 1-5 μl of protein into each drop reservoir to be screened on the plate. Subsequently, the robot loads 1.5 μl of the corresponding screening solution into the drop reservoir atop the protein. The plate is then sealed using transparent tape, and stored at 4 or 20° C. Each plate is observed two days, two weeks, and 1 month after being set. Alternatively, screens may be performed using 0.1-10 μl drops suspended at the interface of two immiscible oils. The protein containing solution has a density intermediate between the two oils and thus floats between them (Chayen N. E.: 1996, Protein Eng. 9:927-29). This procedure may be performed in an automated fashion by an appropriately programmed liquid handling robot, with additional steps being required initially to introduce the oils. No tape is added to facilitate gradual drying out of the drop to promote crystallization.

Having identified conditions that are best suited for further crystal refinement, subsequent plates are set up to explore the affects of variables such as temperature, pH, salt or PEG concentration on crystal size and form, with the intent of establishing conditions where the protein is able to form crystals of suitable size and morphology for diffraction analysis. Each refinement is performed in the sitting drop format in a 24 well Lindbro plate. Each well in the tray contains 500 μl of screening solution, and a 1.5 μl drop of protein diluted with 1.5 μl of the screening solution is set to hang from the siliconized glass cover slip covering the well. Alternatively, refinement steps may be performed using either the machine 96 well plate hanging drop method or the oil suspension method described above.

Crystallization results for one or more polypeptides of the invention are set forth in the applicable Table contained in the Figures.

(b) Co-Crystallization

A variety of methods known in the art may be used for preparation of co-crystals comprising the subject polypeptides and one or more compounds that interact with the subject polypeptides, such as, for example, an inhibitor, co-factor, substrate, polynucleotide, polypeptide, and/or other molecule. In one exemplary method, crystals of the subject polypeptide may be soaked, for an appropriate period of time, in a solution containing a compound that interacts with a subject polypeptide. In another method, solutions of the subject polypeptide and/or compound that interacts with the subject polypeptide may be prepared for crystallization as described above and mixed into the above-described sitting drops. In certain embodiments, the molecule to be co-crystallized with the subject polypeptide may be present in the buffer in the sitting drop prior to addition of the solution comprising the subject polypeptide. In other embodiments, the subject polypeptide may be mixed with another molecule before adding the mixture to the sitting drop. Based on the teachings herein, one of skill in the art may determine the co-crystallization method yielding a co-crystal comprising the subject polypeptide.

(c) Heavy Atom Substitution

For preparation of crystals containing heavy atoms, crystals of the subject polypeptide may be soaked in a solution of a compound containing the appropriate heavy atom for such period as time as may be experimentally determined is necessary to obtain a useful heavy atom derivative for x-ray purposes. Likewise, for other compounds that may be of interest, including, for example, inhibitors or other molecules that interact with the subject polypeptide, crystals of the subject polypeptide may be soaked in a solution of such compound for an appropriate period of time.

(d) Data Collection and Processing

Before data collection may commence, a protein crystal is frozen to protect it from radiation damage. This is accomplished by suspending the crystal in a loop (purchased from Hampton Research) in a stream of dry nitrogen gas at approximately 100 K. The crystals are protected from damage caused by formation of ice crystals (within the lattice or in the liquid surrounding the crystal) upon freezing by supplementing the crystal growth solution with the appropriate cryo-protecting chemical. In some instances, crystals will grow in conditions that provide good cryo-protection, allowing the crystals to be frozen without further modification. In other instances, cryo-protection is achieved by supplementing the crystal growth solution with one or more of the following: 30% volume/volume MPD; 1.2M Na citrate; 30% PEG 400; 4.0M Na Formate; 15% glycerol; 15% ethylene glycol. Alternatively, data may be collected from crystals placed in a thin walled glass capillary and sealed at both ends to protect the crystal from dehydration.

In some cases, data collection is done at the Com-CAT beam-line at the Advanced Photon Source, using a charged coupled device detector. The oscillation method is used. Data is collected for three different wavelengths corresponding to the maximum of anomalous scattering for the appropriate heavy atom, such as selenium, the inflection point and a high energy remote wavelength. Alternatively, data may be collected at only one wavelength corresponding to the maximum of anomalous scattering, with data being collected over a larger range of oscillation angles.

In other cases, data collection is performed in house using a Bruker AXS Proteum R diffractometer. This machine includes a copper rotating anode, Osmic confocal focusing optics and a charge coupled device detector. This data is collected using Cu K_{α} radiation with a wavelength of 1.54 Å, using the oscillation method.

In some instances, data processing is done using the program HKL2000 and data scaling in Scalepack (Z. Otwinowski and W. Minor, Methods in Enzymology vol. 276 p307-326, Academic press). Or, as an alternative, data processing is done using the program Mosfilm and scaling in Scala (Diederichs, K. & Karplus, P. A., Nature Structural Biology, 4, 269-275, 1997).

After scaling, a computer file is obtained which contains the space group, unit cell parameters, and the index, intensity and sigma value for each reflection unique symmetrically. This information forms the raw input of structure determination.

(e) Heavy Atom Substructure, Phasing.

Anomalous scattering sites are found using automated anomalous difference Patterson methods in the program CNX (Brünger A T, Adams P D, Clore G M, DeLano W L, Gros P, Grosse-Kunstleve R W, Jiang J S, Kuszewski J, Nilges M, Pannu N S, Read R J, Rice L M, Simonson T, Warren G L. Acta Crystallogr. D 1998 54 pp 905-21). Alternatively, anomalous scattering sites are found using by real/reciprocal space cycling searches as implemented in shake-and-bake (Weeks C M, DeTitta G T, Hauptman H A, Thuman P, Miller R Acta Crystallogr A 1994; V50: 210-20).

Heavy atom substructure refinement, phase calculation and map calculation are performed in CNX (Brünger AT, et. al. Acta Crystallogr. D 1998 54 pp 905-21), as are density modification (including solvent flipping and non-crystallographic symmetry averaging). In some instances density modification is performed in programs of the CCP4 suite including DM (Collaborative Computational Project, Number 4. 1994. Acta Cryst. D50, 760-763).

The initial protein model may be built in the program TURBO or O. In this process, the crystallographer displays the electron density map on a graphics terminal and interprets the observed density in terms of amino acid residues in the appropriate sequence. Alternatively, QUANTA may be used, which provides an environment for semi-automated model building (Oldfield, T J. Acta Crystallogr D 2001; 57:82-94).

In certain circumstances, the electron density is fully and automatically interpreted in terms of a polypeptide chain using MAID (Levitt, D. G., Acta Crystallogr D 2001 V57:1013-9) or wARP (Perrakis, A., Morris, M. & Lamzin, V. S.; Nature Structural Biology, 1999 V6: 458-463).

(f) Molecular Replacement

In cases where an atomic model sufficiently similar to the structure in question is available, structure solution may proceed by molecular replacement (Rossmann M. G., Acta Crystallogr. A 1990; V46: 73-82). An appropriate search model is identified on the basis of sequence similarity to a suitable target molecule for which a known structure exists in the RCSB protein structure database (http://www.rcsb.org/pdb) or some other (potentially proprietary) database. Alternatively, the molecular replacement solution may be found using genetic algorithms that simultaneously search rotation and translation space, as is done by EPMR (Kissinger C R, Gehlhaar D K, Fogel D B. Acta Crystallogr D 1999; 55: 484-491). The appropriately positioned model may then be refined using rigid body refinement techniques in CNX. This model is then used to calculate model phases, which after solvent flipping in CNX, is used to calculate a map. This map is then used to rebuild the model to better reflect the electron density.

(g) Structure Refinement

The atomic model built by the crystallographer may be used, via theoretical models of how atoms scatter x-rays, to predict the diffraction intensities such a molecule would produce. These predictions can then be compared to the experimentally observed data, allowing the calculation of goodness of fit statistics such as the R-factor. Another important statistic is the R-free, a cross-correlated R-factor calculated using data that has been excluded from model refinement from the beginning. This statistic is free of model bias and can be used, for example, as an objective judge as whether the introduction of extra degrees of freedom into the model is justified (Brunger A T, Clore G M, Gronenborn A M, Saffrich R, Nilges M. Science 1993; 261: 328-31). The model was then iteratively perturbed computationally to maximize the probability that the observed data was produced by the model, as well as to optimize model geometry (as embodied in an energy term) in the process known as refinement. Pragmatically, in order to maximize the computational efficiency convergence radius of refinement, simulated annealing refinement using torsion angle dynamics (in order to reduce the degrees of freedom of motion of the model) (Adams P D, Pannu N S, Read R J, Brunger A T, Acta Crystallogr. D 1999; V55: 181-90). Alternatively, refinement may be performed in the CCP4 program REFMAC, which uses similar procedures (Murshudov, G. N., Vagin, A. A. & Dodson, E. J. (1997). Acta Cryst. D53, 240-253).

Experimental phase information from a MAD experiment may be collected and may be utilized as an additional restraint in the refinement as Hendrickson-Lattman phase probability targets. Individual or group temperature factor refinements may also be performed in CNX.

Automatic water picking routines (implemented in the same package) may be employed to find well ordered solvent molecules, the inclusion of which is justified by a reduction in R-free.

### Example 17

### Annotations

The functional annotation for each of the subject amino acid sequences (predicted) is arrived at by comparing the amino acid sequence of the ORF against all available ORFs in the NCBI database using BLAST. The closest match is selected to provide the probable function of each of the subject amino acid sequences (predicted). Results of this comparison are described above and set forth in the applicable Table contained in the Figures.

The COGs database (Tatusov R L, Koonin E V, Lipman D J. Science 1997; 278 (5338) 631-37) classifies proteins encoded in twenty-one completed genomes on the basis of sequence similarity. Members of the same Cluster of Orthologous Group, (“COG”), are expected to have the same or similar domain architecture and the same or substantially similar biological activity. The database may be used to predict the function of uncharacterised proteins through their homology to characterized proteins. The COGs database may be searched from NCBI's website (http://www.ncbi.nlm.nih.gov/COG/) to determine functional annotation descriptions, such as “information storage and processing” (translation, ribosomal structure and biogenesis, transcription, DNA replication, recombination and repair); “cellular processes” (cell division and chromosome partitioning, post-translational modification, protein turnover, chaperones, cell envelope biogenesis, outer membrane, cell motility and secretion, inorganic ion transport and metabolism, signal transduction mechanisms); or “metabolism” (energy production and conversion, carbohydrate transport and metabolism, amino acid transport and metabolism, nucleotide transport and metabolism, coenzyme metabolism, lipid metabolism). For certain polypeptides, there is no entry available. Results of this analysis are described above and set forth in the applicable Table contained in the Figures.

### Example 18

### Essential Gene Analysis

Each of the subject amino acid sequences (predicted) is compared to a number of publicly available “essential genes” lists to determine whether that protein is encoded by an essential gene. An example of such a list is descended from a free release at the www.shigen.nig.ac.ip PEC (profiling of E. coli chromosome) site, http://www.shigen.nig.ac.jp/ecoli/pec/. The list is prepared as follows: a wildcard search for all genes in class “essential” yields the list of essential E. coli proteins encoded by essential genes, which number 230. These 230 hits are pruned by comparing against an NCBI E. coli genome. Only 216 of the 230 genes on the list are found in the NCBI genome. These 216 are termed the essential-216-ecoli list. The essential-216-ecoli list is used to garner “essential” genes lists for other microbial genomes by blasting. For instance, formatting the 216-ecoli as a BLAST database, then BLASTing a genome (e.g. S. aureus) against it, elucidates all S. aureus genes with significant homology to a gene in the 216-essential list. Each of the subject amino acid sequences (predicted) is compared against the appropriate list and a match with a score of e⁻²⁵ or better is considered an essential gene according to that list. In addition to the list described above, other lists of essential genes are publicly available or may be determined by methods disclosed publicly, and such lists and methods are considered in deciding whether a gene is essential. See, for example, Thanassi et al., Nucleic Acids Res 2002 Jul. 15; 30(14):3152-62; Forsyth et al., Mol Microbiol 2002 March; 43(6):1387-400; Ji et al., Science 2001 Sep. 21; 293(5538):2266-9; Sassetti et al., Proc Natl Acad Sci USA 2001 Oct. 23; 98(22):12712-7; Reich et al., J Bacteriol 1999 August; 181(16):4961-8; Akerley et al., Proc Natl Acad Sci USA 2002 Jan. 22; 99(2):966-71). Also, other methods are known in the art for determing whether a gene is essential, such as that disclosed in U.S. patent application Ser. No. 10/202,442 (filed Jul. 24, 2002). The conclusion as to whether the gene encoding a subject amino acid sequence (predicted) is essential is set forth in the applicable Table contained in the Figures.

### Example 19

### PDB Analysis

Each of the subject amino acid sequences is compared against the amino acid sequences in a database of proteins whose structures have been solved and released to the PDB (protein data bank). The identity/information about the top PDB homolog (most similar “hit”, if any, a PDB entry is only considered a hit if the score is e⁻⁴ or better) is annotated, and the percent similarity and identity between a subject amino acid sequence (predicted) and the closest hit is calculated, with both being indicated in the applicable Table contained in the Figures.

### Example 20

### Virtual Genome Analysis

VGDB or VG is a queryable collection of microbial genome databases annotated with biophysical and protein information. The organisms present in VG include:

| | | | | Genome |
|---|---|---|---|---|
| File | GRAM | Species | Source | file date |
| ecoli.faa | G− | Escherichia coli | NCBI | Nov. 18, 1998 |
| hpyl.faa | G− | Helicobacter pylori | NCBI | Apr. 19, 1999 |
| paer.faa | G− | Pseudomonas | NCBI | Sep. 22, 2000 |
| | | aeruginosa | | |
| ctra.faa | G− | Chlamydia trachomatis | NCBI | Dec. 22, 1999 |
| hinf.faa | G− | Haemophilus influenzae | NCBI | Nov. 26, 1999 |
| nmen.faa | G− | Neisseria meningitidis | NCBI | Dec. 28, 2000 |
| rpxx.faa | G− | Rickettsia prowazekii | NCBI | Dec. 22, 1999 |
| bbur.faa | G− | Borrelia burgdorferi | NCBI | Nov. 11, 1998 |
| bsub.faa | G+ | Bacillus subtilis | NCBI | Dec. 1, 1999 |
| staph.faa | G+ | Staphylococcus aureus | TIGR | Mar. 8, 2001 |
| spne.faa | G+ | Streptococcus | TIGR | Feb. 22, 2001 |
| | | pneumoniae | | |
| mgen.faa | G+ | Mycoplasma genitalium | NCBI | Nov. 23, 1999 |
| efae.faa | G+ | Enterococcus faecalis | TIGR | Mar. 8, 2001 |

The VGDB comprises 13 microbial genomes, annotated with biophysical information (pI, MW, etc), and a wealth of other information. These 13 organism genomes are stored in a single flatfile (the VGDB) against which PSI-blast queries can be done.

Each of the subject amino acid sequences (predicted) is queried against the VGDB to determine whether this sequence is found, conserved, in many microbial genomes. There are certain criteria that must be met for a positive hit to be returned (beyond the criteria inherent in a basic PSI-blast). When an ORF is queried it may have a maximum of 13 VG-organism hits. A hit is classified as such as long as it matches the following criteria: Minimum Length (as percentage of query length): 75 (Ensure hit protein is at least 75% as long as query); Maximum Length (as percentage of query length): 125 (Ensure hit protein is no more than 125% as long as query); eVal:−10 (Ensure hit has an e-Value of e-10 or better); Id %:>:25 (Ensure hit protein has at least 25% identity to query). The e-Value is a standard parameter of BLAST sequence comparisons, and represents a measure of the similarity between two sequences based on the likelihood that any similarities between the two sequences could have occurred by random chance alone. The lower the e-Value, the less likely that the similarities could have occurred randomly and, generally, the more similar the two sequences are. The organisms having positive hits based on the foregoing for each of the subject amino acid sequences (predicted) are listed in the applicable Table contained in the Figures.

### Example 21

### Epitopic Regions

The three most likely epitopic regions of each of the subject amino acid sequences (predicted) are predicted using the semi-empirical method of Kolaskar and Tongaonkar (FEBS Letters 1990 v276 172-174), the software package called Protean (DNASTAR), or MacVectors's Protein analysis tools (Accerlyrs). The antigenic propensity of each amino acid is calculated by the ratio between frequency of occurrence of amino acids in 169 antigenic determinants experimentally determined and the calculated frequency of occurrence of amino acids at the surface of protein. The results of these bioinformatics analyses are presented in the applicable Table contained in the Figures.

Equivalents

The present invention provides among other things, proteins, protein structures and protein-protein interactions. While specific embodiments of the subject invention have been discussed, the above specification is illustrative and not restrictive. Many variations of the invention will become apparent to those skilled in the art upon review of this specification. The full scope of the invention should be determined by reference to the claims, along with their full scope of equivalents, and the specification, along with such variations.

All publications and patents mentioned herein, including those items listed below, are hereby incorporated by reference in their entirety as if each individual publication or patent was specifically and individually indicated to be incorporated by reference. In case of conflict, the present application, including any definitions herein, will control. To the extent that any U.S. Provisional Patent Applications to which this patent application claims priority incorporate by reference another U.S. Provisional Patent Application, such other U.S. Provisional Patent Application is not incorporated by reference herein unless this patent application expressly incorporates by reference, or claims priorty to, such other U.S. Provisional Patent Application.

Also incorporated by reference in their entirety are any polynucleotide and polypeptide sequences which reference an accession number correlating to an entry in a public database, such as those maintained by The Institute for Genomic Research (TIGR) (www.tigr.org) and/or the National Center for Biotechnology Information (NCBI) (www.ncbi.nlm.nih.gov).

Also incorporated by reference are the following: WO 00/45168, WO 00/79238, WO 00/77712, EP 1047108, EP 1047107, WO 00/72004, WO 00/73787, WO00/67017, WO 00/48004, WO 01/48209, WO 00/45168, WO 00/45164, U.S. Ser. No. 09/720,272; PCT/CA99/00640; U.S. patent application Ser. No. 10/097,125 (filed Mar. 12, 2002); Ser. No. 10/097,193 (filed Mar. 12, 2002); Ser. No. 10/202,442 (filed Jul. 24, 2002); Ser. No. 10/097,194 (filed Mar. 12, 2002); Ser. No. 09/671,817 (filed Sep. 17, 2000); Ser. No. 09/965,654 (filed Sep. 27, 2001); Ser. No. 09/727,812 (filed Nov. 30, 2000); 60/370,667 (filed Apr. 8, 2002); a utility patent application entited “Methods and Appartuses for Purification” (filed Sep. 18, 2002); U.S. Pat. Nos. 6,451,591; 6,254,833; 6,232,114; 6,229,603; 6,221,612; 6,214,563; 6,200,762; 6,171,780; 6,143,492; 6,124,128; 6,107,477; D428157; 6,063,338; 6,004,808; 5,985,214; 5,981,200; 5,928,888; 5,910,287; 6,248,550; 6,232,114; 6,229,603; 6,221,612; 6,214,563; 6,200,762; 6,197,928; 6,180,411; 6,171,780; 6,150,176; 6,140,132; 6,124,128; 6,107,066; 6,270,988; 6,077,707; 6,066,476; 6,063,338; 6,054,321; 6,054,271; 6,046,925; 6,031,094; 6,008,378; 5,998,204; 5,981,200; 5,955,604; 5,955,453; 5,948,906; 5,932,474; 5,925,558; 5,912,137; 5,910,287; 5,866,548; 6,214,602; 5,834,436; 5,777,079; 5,741,657; 5,693,521; 5,661,035; 5,625,048; 5,602,258; 5,552,555; 5,439,797; 5,374,710; 5,296,703; 5,283,433; 5,141,627; 5,134,232; 5,049,673; 4,806,604; 4,689,432; 4,603,209; 6,217,873; 6,174,530; 6,168,784; 6,271,037; 6,228,654; 6,184,344; 6,040,133; 5,910,437; 5,891,993; 5,854,389; 5,792,664; 6,248,558; 6,341,256; 5,854,922; and 5866343.
Onesti, S., (2000) Biochemistry 39:12853-12861; Fishman, R., et al. (2001) Acta Crystallographica D Biological Crystallography 57:1534-1544; Nakama, T., et al. (2001) Journal of Biological Chemistry 276:47387-47393; Brown, M. J. B., et al. (2000) Biochemistry 39:6003-6011; Lee, J., et al (2001) Bioorganic & Medicinal Chemistry Letters 11:965-968; and Xiang, Y. Y., (1999) Bioorganic & Medicinal Chemistry Letters 9:375-380.
Lonsdale, et al, (2001), DDT 6:537-544; Rock, C. & Cronan, J. (1996) Biochimica et Biophysica Acta 1302:1-16; Jackowski, S. (1992); and Heath, et al, (1996), J. Biol. Chem. 271:1833-1836.
Huenges, M., et al. (1998) EMBO J. 17:4092-4100; Berglechner, F., et al. (1997) J. Biochem. 248:338-346; Altieri, A. S., et al. (1997) FEBS Lett. 415:221-226; Nodwell, J. R. and Greenblatt, J. (1993) Cell 72:261-268; Mogridge, J., et al. (1995) Genes Dev. 9:2831-2845; Mogridge, J. and Greenblatt, J. (1998) J. Bacteriol. 180:2248-2252; Gopal, B., et al. (2000) Acta Crystallogr. D. Biol. Crystallogr. 56:64-66; Mogridge, J., et al. (1998) J. Biol. Chem. 273:4143-4148; Gopal, B., et al. (2000) Nat. Struct. Biol. 7:475-478; and Altieri, A. S., et al. (2000) Nat. Struct. Biol. 7:470-474).
Nature (1996) 379(6565):511-8; Nuc; Acid Res. (2001) 29:774-782; FEBS Lett (1991) 288(1-2):98-100; J Mol Biol (1990) 215(1):41-51; Mol Gen Genet (1999) 260(6):603-7; J Mol Biol (1990) 215(1):41-51.
Trach K, Hoch J A. (1989) J Bacteriol 171(3):1362-71; Kobayashi G, et al. (2001) Mol Microbiol. 41(5):1037-1051; Mittenhuber G. (2001) J Mol Microbiol Biotechnol. 3(1):21-35; Scott J M, et al. (2000) J. Bacteriol. 182(10):2771-7; Welsh KM, et al. (1994) J. Bacteriol. 176(23):7161-8; Kok J, et al. (1994) J. Bacteriol. 176(23):7155-60; Vidwans, S. J., et al. (1995) 177:3308-3311; Okamoto, S., and K. Ochi. (1998) Mol. Microbiol. 30:107-119; Janelle M. Scott and W. G. Haldenwang, (1999) J. Bacteriol. 181: 9193-9; and Czyz A, et al. (2001) Microbiology. 147(Pt 1): 183-91.
Zhou, J., et al. (1997) Mol. Microbiol. 23:799-812; Maclean, S. A. et al. (2000) Acta Cryst. D56, 512-515; Anton I A, and Coggins J R. (1988) Biochem J 1988 249(2):319-26; Dowsett J R, et al. (1972) Biochim Biophys Acta 276(2):344-9; and Baillie A C, et al. (1972) Biochem J 126(3):21.
Jackowski, S (1992); Lonsdale, et al, (2001) DDT 6:537-544; Rock, C. & Cronan, J. (1996) Biochimica et Biophysica Acta 1302:1-16; Tsay, et al. (1992) J Bacteriol 174:508-13; and Heath, et al, (1996) J. Biol. Chem. 271:1833-1836.
J. Bacteriol. (1999) 181(4):1281-91; Janosi, L. et al. (1998) EMBO J. 17:1141-1151; Teyssier, E. et al. (1998) CSHL, Translational Control Mtg. September 9-12, (Abstract); Johnston, M. et al. (1994) Science 265:2077-2082; Pious, D. A. and Hawley, P. (1972) Pediat. Res. 6:687-692; and Borst, P. and Grivell, L. A. (1998) FEBS Lett. 13:73-88.
Gusarov, I. and Nudler, E. (2001) Cell 107:437-449; Mogridge, J., et al. (1995) Genes Dev. 9, 2831-2845; Gopal, B., et al. (2001) Acta Crystallogr. D. Biol. Crystallogr. 57:1187-1188; Mah, T. F., et al. (1999) Mol. Microbiol. 34:523-537; Liu, K. and Hanna, M. M. (1995) Proc. Natl. Acad. Sci. U.S.A. 92:5012-5016.
Mittenhuber, G. (2001). J Mol Microbiol Biotechnol. 1:21-35; Hang et al. (2001). Eur J. Biochem. 21:5570-5577; Chen et al. (1999). PNAS 96:8396-8401; Meier et al. (2000). Microbiology 146:1071-1083; and Britton et al. (1997). J. Bacteriol. 179:4575-4582.
Pichoff, S. and Lutkenhaus, J. (2002) The EMBO Journal. 21, 685-693; Lowe, J. and van den Ent, F. (2001) Biochimie. 83, 117-120; and Donachie, W. D. (2001) Molecular Microbiology. 40, 779-785.
Doublet et al., (1993) J. Bacteriol. 175: 2970-2979; Doublet et al., (1992) J. Bacteriol. 174: 5772-5779; Doublet et al., (1994) Biochemistry 33:5285-5290; Hwang et al., (1999) Nat Struct Biol 5:422-66; Glavas and Tanner (1999) Biochemistry 13:4106-13; and Glavas and Tanner (2001) Biochemistry 21: 6199-6204.
Darst et al., Cell (1999) 98:811-824
U.S. Pat. No. 6,225,076; Darst et al., (2001) Cell 104: 901-912; Kuznedelov et al. (2002) Science 295:855-857; Darst et al., (1999) Cell 98:811-824; and Naryshlidna et al., (2001) J. Biol. Chem. 276:13308-13313.

We claim:

1. A composition comprising an isolated, recombinant polypeptide, wherein the polypeptide comprises: (a) an amino acid sequence set forth in SEQ ID NO: 5 or SEQ ID NO: 7; (b) an amino acid sequence having at least about 95% identity with the amino acid sequence set forth in SEQ ID NO: 5 or SEQ ID NO: 7; or (c) an amino acid sequence encoded by a polynucleotide that hybridizes under stringent conditions to the complementary strand of a polynucleotide having SEQ ID NO: 4 or SEQ ID NO: 6 and has at least one biological activity of GTP-binding protein Era from P. aeruginosa; and wherein the polypeptide of (a), (b) or (c) is at least about 90% pure in a sample of the composition.

2. The composition of claim 1, wherein at least about two-thirds of the polypeptide in the sample is soluble.

3. The composition of claim 1, wherein the polypeptide is fused to at least one heterologous polypeptide that increases the solubility or stability of the polypeptide.

4. The composition of claim 1, which further comprises a matrix suitable for mass spectrometry.

5. The composition of claim 4, wherein the matrix is a nicotinic acid derivative or a cinnamic acid derivative.

6. The composition of claim 1, wherein said polypeptide of (a), (b) or (c) is labeled with a heavy atom.

7. The composition of claim 6, wherein the heavy atom is one of the following: cobalt, selenium, krypton, bromine, strontium, molybdenum, ruthenium, rhodium, palladium, silver, cadmium, tin, iodine, xenon, barium, lanthanum, cerium, praseodymium, neodymium, samarium, europium, gadolinium, terbium, dysprosium, holmium, erbium, thulium, ytterbium, lutetium, tantalum, tungsten, rhenium, osmium, iridium, platinum, gold, mercury, thallium, lead, thorium and uranium.

8. The composition of claim 6, wherein the polypeptide is labeled with seleno-methionine.

9. The composition of claim 6, further comprising a cryo-protectant.

10. The composition of claim 9, wherein the cryo-protectant is one of the following: methyl pentanediol, isopropanol, ethylene glycol, glycerol, formate, citrate, mineral oil and a low-molecular-weight polyethylene glycol.

11. The composition of claim 1, wherein said composition is a crystallized, recombinant polypeptide; wherein the polypeptide of (a), (b) or (c) is in crystal form.

12. A crystallized complex comprising the crystallized, recombinant polypeptide of claim 11 and a co-factor, wherein the complex is in crystal form.

13. A crystallized complex comprising the crystallized, recombinant polypeptide of claim II and a small organic molecule, wherein the complex is in crystal form.

14. The crystallized, recombinant polypeptide of claim 13, which diffracts x-rays to a resolution of about 3.5 Å or better.

15. The crystallized, recombinant polypeptide of claim 13, wherein the polypeptide comprises at least one heavy atom label.

16. The crystallized, recombinant polypeptide of claim 13, wherein the polypeptide is labeled with seleno-methionine.

17. A method for designing a modulator for the prevention or treatment of a disease or disorder associated with the species of origin for a polypeptide of the composition of claim 1, comprising:
(a) providing a three-dimensional structure for a crystallized, recombinant polypeptide of claim 13;
(b) identifying a potential modulator for the prevention or treatment of said disease or disorder by reference to the three-dimensional structure;
(c) contacting a polypeptide of the composition of claim 1 or its species of origin with the potential modulator; and
(d) assaying the activity of the polypeptide or determining the viability of the species of origin after contact with the modulator, wherein a change in the activity of the polypeptide or the viability of said species indicates that the modulator may be useful for prevention or treatment of a disease or disorder associated with the species of origin for the polypeptide.

18. A composition of claim 1, wherein said polypeptide of (a), (b) or (c) is enriched in at least one NMR isotope.

19. The composition of claim 18, wherein the NMR isotope is one of the following: hydrogen-1 (1H), hydrogen-2 (2H), hydrogen-3 (3H), phosphorous-31 (31P), sodium-23 (23Na), nitrogen-14 (14N), nitrogen-15 (¹⁵N), carbon-13 (¹³C) and fluorine-19 (19F).

20. The composition of of claim 19, further comprising a deuterium lock solvent.

21. The composition of claim 20, wherein the deuterium lock solvent is one of the following: acetone (CD3COCD3), chloroform (CDCl3), dichloro methane (CD2C12), methylnitrile (CD3CN), benzene (C6D6), water (D2O), diethylether ((CD3CD2)2O), dimethylether ((CD3)2O), N,N-dimethylformamide ((CD3)2NCDO), dimethyl sulfoxide (CD3SOCD3), ethanol (CD3CD20D), methanol (CD3OD), tetrahydrofuran (C4D8O), toluene (C6D5CD3), pyridine (C5D5N) and cyclohexane (C6H12).

22. The composition of claim 18, which is contained within an NMR tube.

23. A method for identifying small molecules that bind to an isotopically labeled polypeptide of the composition of claim 18, comprising:
(a) generating a first NMR spectrum of said isotopically labeled polypeptide;
(b) exposing the polypeptide to one or more small molecules;
(c) generating a second NMR spectrum of the polypeptide which has been exposed to one or more small molecules; and
(d) comparing the first and second spectra to determine differences between the first and the second spectra, wherein the differences are indicative of one or more small molecules that have bound to the polypeptide.

24. A host cell comprising a nucleic acid encoding a polypeptide comprising the composition of claim 1; wherein a culture of the host cell produces at least about 1 mg of the polypeptide per liter of culture and the polypeptide is at least about one-third soluble as measured by gel electrophoresis.

25-63. (canceled)
